(19) United States
(12) Patent Application Publication
(10) Pub. No.: US 2026/0241032 A1
(43) Pub. Date: Aug. 20, 2026

(54) COMPOSITIONS AND METHODS FOR THE TARGETING OF PCSK9

(71) Applicant: Scribe Therapeutics Inc., Alameda, CA (US)

(72) Inventors: Addison WRIGHT, El Cerrito, CA (US); Sean HIGGINS, Alameda, CA (US); Maroof ADIL, Davis, CA (US); Cole URNES, Los Angeles, CA (US); Santhosh KARANTH, Union City, CA (US); Brett T. STAAHL, Tiburon, CA (US); Sarah DENNY, San Francisco, CA (US); Benjamin OAKES, El Cerrito, CA (US); Oleh KRUPA, Oakland, CA (US); Matthew HARMS, Alameda, CA (US); Manuel MOHR, Berkeley, CA (US); Wenyuan ZHOU, Dublin, CA (US); Kian TAYLOR, Atlanta, GA (US)

(21) Appl. No.: 18/872,544

(22) PCT Filed: Jun. 6, 2023
(86) PCT No.: PCT/US2023/067985

§ 371 Date: Dec. 6, 2024

Related U.S. Application Data
(60) Provisional application No. 63/349,981, filed on Jun. 7, 2022.
(60) Provisional application No. 63/492,880, filed on Mar. 29, 2023.
(60) Provisional application No. 63/505,660, filed on Jun. 1, 2023.

(51) Int. Cl. A61K 48/00; A61K 9/127; A61K 9/51; A61P 3/06; C12N 9/22; C12N 9/64; C12N 15/11; C12N 15/85; C12N 15/88

(52) U.S. Cl. CPC A61K 48/005; A61K 9/127; A61K 9/5123; A61P 3/06; C12N 9/226; C12N 9/6424; C12N 15/111; C12N 15/85; C12N 15/88; C07K 2319/09; C12N 2310/20; C12Y 304/21

(57) ABSTRACT

Provided herein are systems comprising Class 2, Type V CRISPR proteins and guide nucleic acids (gRNA) useful in the modification of a PCSK9 gene. The systems are also useful for introduction into cells, for example eukaryotic cells. Also provided are methods of using such systems to modify cells having such mutations.

### CROSS-REFERENCE TO RELATED APPLICATIONS

This application claims priority to, and benefit of, U.S. Provisional Application Nos. 63/349,981 filed on Jun. 7, 2022, and 63/492,880, filed on Mar. 29, 2023, and 63/505,660, filed on Jun. 1, 2023, the contents of each of which are incorporated by reference herein in their entireties.

### REFERENCE TO AN ELECTRONIC SEQUENCE LISTING

The contents of the electronic sequence listing (SCRB_042_01WO_SeqList_ST26.xml; Size: 14,072,595 bytes; and Date of Creation: May 30, 2023) are herein incorporated by reference in its entirety.

### BACKGROUND

In mammals, cholesterol is transported within lipoproteins via emulsification. The lipoprotein particles are classified based on their density: low-density lipoproteins (LDL), very low-density lipoproteins (VLDL), high-density lipoproteins (HDL), and chylomicrons. Surface LDL receptors are internalized during cholesterol absorption. A cell with abundant cholesterol will have its LDL receptor synthesis blocked to prevent new cholesterol in LDL particles from being taken up. Conversely, LDL receptor synthesis is promoted when a cell is deficient in cholesterol. When the process is unregulated, excess LDL particles will travel in the blood without uptake by an LDL receptor. LDL particles in the blood are oxidized and taken up by macrophages, which then become engorged and form foam cells. These foam cells can become trapped in the walls of blood vessels and contribute to atherosclerotic plaque formation, which is one of the main causes of heart attacks, strokes, and other serious medical problems.

The liver protein proprotein convertase subtilisin/kexin Type 9 (PCSK9) is a secreted, globular, auto-activating serine protease that binds to the low-density lipoprotein receptor (LDL-R) during endocytosis of LDL particles, preventing recycling of the LDL-R to the cell surface and leading to reduction of LDL-cholesterol clearance. PCSK9 binds to the LDL-R (through the EGF-A domain), preventing the conformational change of the receptor-ligand complex, which redirects the LDL-R to the lysosome instead. As the receptor for low-density lipoprotein particles (LDL) typically transports thousands of fat molecules (including cholesterol) per particle within extracellular fluid, blocking or inhibiting the function of PCSK9 to boost LDL-R-mediated clearance of LDL cholesterol can lower LDL particle concentrations. PCSK9 is expressed mainly in the liver, the intestine, the kidney, and the central nervous system, but is also highly expressed in arterial walls such as endothelium, smooth muscle cells, and macrophages, with a local effect that can regulate vascular homeostasis and atherosclerosis.

PCSK9 is a member of the proprotein convertase (PC) family and its gene is mutated in ~2% to 3% of individuals with familial hypercholesterolemia (FH) (Sepideh Mikaeeli, S., et al. Functional analysis of natural PCSK9 mutants in modern and archaic humans. FEBS J. 2019 Aug. 6. doi: 10.1111/febs.15036). Researchers have identified several PCSK9 mutations that cause an inherited form of high cholesterol (hypercholesterolemia). These mutations change a single protein building block (amino acid) in the PCSK9 protein. Researchers describe the mutations responsible for hypercholesterolemia as “gain-of-function” because they appear to enhance the activity of the PCSK9 protein or give the protein a new, atypical function (Blesa, S., et al. A New PCSK9 Gene Promoter Variant Affects Gene Expression and Causes Autosomal Dominant Hypercholesterolemia. J. Clin. Endocrinol. & Metab. 93: 3577 (2008)). The overactive PCSK9 protein substantially reduces the number of low-density lipoprotein receptors on the surface of liver cells. With fewer receptors to remove low-density lipoproteins from the blood, people with gain-of-function mutations in the PCSK9 gene have very high blood cholesterol levels. Autosomal dominant hypercholesterolemia (ADH) is a genetic disorder characterized by increased low-density lipoprotein (LDL)-cholesterol levels, leading to high risk of premature cardiovascular disease. Approximately 10 mutations in PCSK9 have been identified as a cause of the disease in different populations. All known mutations in PCSK9 causing hypercholesterolemia produce an increase in the enzymatic activity of this protease (Bleasa, S., 2008). In addition, mutations in PCSK9 can lead to autosomal dominant familial hypobetalipoproteinemia, which can lead to hepatic steatosis, cirrhosis, and other disorders.

The advent of CRISPR/Cas systems and the programmable nature of these minimal systems has facilitated their use as a versatile technology for genomic manipulation and engineering. However, current methods of generating PCSK9 protective variants and loss-of-function mutants in vivo have been ineffective due to the large number of cells that need to be modified to modulate cholesterol levels. Other concerns involve off-target effects, genome instability, or oncogenic modifications that may be caused by genome editing, as well as a lack of safe delivery modalities for gene-editing systems. Thus, there remains a need for improved compositions and methods to regulate PCSK9.

### SUMMARY

The present disclosure provides systems comprising or encoding modified Class 2, Type V CRISPR proteins and guide nucleic acids used in the modification of proprotein convertase subtilisin/kexin Type 9 (PCSK9) gene target nucleic acid sequences. The Class 2, Type V CRISPR proteins and guide nucleic acids can be modified for passive entry into target cells. The Class 2, Type V CRISPR proteins and guide nucleic acids are useful in a variety of methods for target nucleic acid modification of PCSK9, which methods are also provided. The present disclosure also provides vectors and lipid nanoparticles (LNP) encoding or encapsulating the Class 2, Type V CRISPR proteins and guide nucleic acids components for the delivery of the systems to cells for the modification of the PCSK9 target nucleic acid.

The disclosure provides pharmaceutical compositions comprising the systems, nucleic acids, LNP and vectors described herein.

The present disclosure also provides methods for treating subjects having a PCSK9-related disease. In some embodiments, the compositions and methods have utility in subjects having a metabolic disorder such as, but not limited to familial hypercholesterolemia or familial hypobetalipoproteinemia.

The present disclosure provides compositions for use in methods of treating subjects having a PCSK9-related disease. In some embodiments, the composition comprises modified Class 2, Type V CRISPR proteins and guide nucleic acids for use in the modification of PCSK9 gene target nucleic acid sequences in a subject.

In other embodiments, provided herein are compositions comprising Class 2, Type V CRISPR:gRNA systems or vectors comprising or encoding Class 2, Type V CRISPR:gRNA systems for use in the manufacture of a medicament for the treatment of a PCSK9-related disease in a subject in need thereof.

In some embodiments, the PCSK9 gene comprises one or more mutations, for example amino acid substitutions selected from the group consisting of S127R, D129G, F216L, D374H, and D374Y relative to the sequence of SEQ ID NO: 543.

The disclosure provides methods of modifying a PCSK9 gene in a population of cells, the method comprising introducing into cells of the population the systems, nucleic acids, LNP, vectors and/or pharmaceutical compositions described herein.

In some embodiments, the modified Class 2, Type V CRISPR proteins and guide nucleic acids comprise CasX variant proteins and/or CasX variant guide nucleic acids as described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The contents of WO 2020/247882, WO 2020/247883, WO 2021/113772, WO 2021/050601, WO 2021/050593, WO 2021/113763, WO 2021/113769, WO 2022/125843, WO 2022/120094, WO 2022/120095, WO 2022/261150, WO 2022/261149, WO 2023/049872, WO 2022/261148, WO 2021/188729, WO 2022/120089, WO 2023/049742, and PCT/US2023/067791, filed on Jun. 1, 2023, which disclose CasX variants and gRNA variants, and methods of delivering same, and WO 2021/142342, which discloses systems and methods of modifying PCSK9, are hereby incorporated by reference in their entirety.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows the total percentage of editing of the PCSK9 locus in HEK293T cells by an exemplary modified Class 2, Type V CRISPR protein of the disclosure, an engineered CasX, as described in Example 1.

FIG. 2 is a graph of results of editing assayed by next generation sequencing (NGS) of a CasX at the PCSK9 locus in Hep2G cells showing total editing percentage as described in Example 2. Non-targeting refers to a non-targeting spacer sequence in the gRNA.

FIG. 3 is a graph of results of editing assayed by NGS of a CasX at the PCSK9 locus in AML12 cells showing total editing percentage as described in Example 3.

FIG. 4 is a bar chart showing the quantification of normalized percent editing measured for the indicated targeting spacers as indel rate detected by NGS at the PCSK9 locus in HEK293T cells at two days post-transfection, as described in Example 4. Editing rates were normalized by transfection efficiency determined by mScarlet expression for the indicated experimental condition. Human spacers having consensus sequence with the non-human primate (NHP) species are depicted in gray.

FIG. 5 is a plot displaying an inverse correlation between editing at the PCSK9 locus (measured as percent editing quantified as indel rate detected by NGS) and secreted PCSK9 levels (measured by ELISA) in HepG2 cells transduced with AAV particles containing constructs encoding the indicated CasX variant with the PCSK9-targeting gRNA, as described in Example 5. Untreated cells served as experimental control.

FIG. 6A is a schematic illustrating versions 1-3 (“V1-V3”) of chemical modifications made to gRNA scaffold variant 235, as described in Example 6. Structural motifs are highlighted. Standard ribonucleotides are depicted as open circles, and 2′OMe-modified ribonucleotides are depicted as black circles. Phosphorothioate bonds are indicated with * below or beside the bond. For the V2 profile (middle), the addition of three 3′ uracils (3′UUU) is annotated with “U”s in the relevant circles at the far right.

FIG. 6B is a schematic illustrating versions 4-6 (“V4-V6”) of chemical modifications made to gRNA scaffold variant 235, as described in Example 6. Structural motifs are highlighted. Standard ribonucleotides are depicted as open circles, and 2′OMe-modified ribonucleotides are depicted as black circles. Phosphorothioate bonds are indicated with * below or beside the bond.

FIG. 7 is a plot illustrating the quantification of percent knockout of beta-2-microglobulin (B2M) in HepG2 cells co-transfected with 100 ng of CasX 491 mRNA and with the indicated doses of end-modified (v1, also referred to as V1) or unmodified (v0) B2M-targeting gRNAs with spacer 7.37, as described in Example 6. Editing level was determined by flow cytometry as the population of cells with loss of surface presentation of the human leukocyte antigen (HLA) complex due to successful editing at the B2M locus.

FIG. 8 is a schematic illustrating versions 7-9 (“V7-V9”) of chemical modifications made to gRNA scaffold variant 316, as described in Example 6. Structural motifs are highlighted. Standard ribonucleotides are depicted as open circles, and 2′OMe-modified ribonucleotides are depicted as black circles. Phosphorothioate bonds are indicated with * below or beside the bond.

FIG. 9A is a schematic of gRNA scaffold variant 174 (SEQ ID NO: 464), as described in Example 6. Structural motifs are highlighted.

FIG. 9B is a schematic of gRNA scaffold variant 235 (SEQ ID NO: 465), as described in Example 6. Highlighted structural motifs are the same as in FIG. 9A. The differences between variant 174 and variant 235 lie in the extended stem motif and several single-nucleotide changes (indicated with asterisks). Variant 316 (SEQ ID NO: 466) maintains the shorter extended stem from variant 174 but harbors the four substitutions found in scaffold 235.

FIG. 9C is a schematic of gRNA scaffold variant 316 (SEQ ID NO: 466), as described in Example 6. Highlighted structural motifs are the same as in FIG. 9A. Variant 316 maintains the shorter extended stem from variant 174 (FIG. 9A) but harbors the four substitutions found in scaffold 235 (FIG. 9B).

FIG. 10 is a plot displaying a correlation between indel rate (depicted as edit fraction) at the PCSK9 locus as measured by NGS (x-axis) and secreted PCSK9 levels (ng/ml) detected by ELISA (y-axis) in HepG2 cells lipofected with CasX 491 mRNA and PCSK9-targeting gRNAs containing the indicated scaffold variant and spacer combination, as described in Example 6.

FIG. 11A is a plot depicting the results of an editing assay measured as indel rate detected by NGS (y-axis) at the human B2M locus in HepG2 cells treated with the indicated doses (x-axis) of LNPs formulated with CasX 491 mRNA and the indicated B2M-targeting gRNA, as described in Example 6.

FIG. 11B is a plot illustrating the quantification of percent knockout of B2M in HepG2 cells treated with the indicated doses of LNPs formulated with CasX 491 mRNA and the indicated B2M-targeting gRNA, as described in Example 6. Editing level was determined by flow cytometry, as the population of cells with successful editing of the B2M locus did not have surface presentation of the HLA complex.

FIG. 12A is a plot depicting the results of an editing assay. The y-axis shows indel rate detected by NGS at the mouse ROSA26 locus in Hepa1-6 cells treated with the indicated doses of LNPs (x-axis) formulated with CasX 676 mRNA #2 and the indicated ROSA26-targeting gRNA with either the v1 or v5 modification profile, as described in Example 6.

FIG. 12B is a plot illustrating the quantification of percent editing measured as indel rate detected by NGS at the ROSA26 locus in mice treated with LNPs formulated with CasX 676 mRNA #2 and the indicated chemically-modified ROSA26-targeting gRNA, as described in Example 6.

FIG. 13 is a bar graph showing the results of an editing assay measured as indel rate detected by NGS in mice treated with LNPs formulated with CasX 676 mRNA #1 and the indicated chemically-modified PCSK9-targeting gRNA, as described in Example 6. Untreated mice served as experimental control.

FIG. 14A is a schematic illustrating versions 1-3 (“V1-V3”) of chemical modifications made to gRNA scaffold variant 316, as described in Example 6. Structural motifs are highlighted. Standard ribonucleotides are depicted as open circles, and 2′OMe-modified ribonucleotides are depicted as black circles. Phosphorothioate bonds are indicated with * below or beside the bond. For the V2 profile (middle), the addition of three 3′ uracils (3′UUU) is annotated with “U”s in the relevant circles at the far right.

FIG. 14B is a schematic illustrating versions 4-6 (“V4-V6”) of chemical modifications made to gRNA scaffold variant 316, as described in Example 6. Structural motifs are highlighted. Standard ribonucleotides are depicted as open circles, and 2′OMe-modified ribonucleotides are depicted as black circles. Phosphorothioate bonds are indicated with * below or beside the bond.

FIG. 15 is a bar graph of editing outcomes at the mouse ROSA26 locus assayed in genomic DNA (gDNA) extracted from the liver tissue of mice injected with an escalating dose of AAV, as described in Example 8. Editing outcomes proportionally increased with increase in dose and duration with 3.0e+11vg (vector genome) dose shows saturation at 1-week and 4-weeks post-harvest in liver.

FIG. 16 is a bar graph showing the quantification of percent editing, measured as indel rate detected by NGS, at the ROSA26 locus in livers harvested from mice 7 days post-injection with LNPs encapsulating CasX 676 mRNA #1 with a ROSA26-targeting gRNA, as described in Example 9. Various doses of LNP are shown. Data are presented as mean±standard deviation, with N=3 animals per experimental group. P-values shown were determined for the indicated group comparisons using a one-way ANOVA with Tukey's correction for multiple comparisons.

FIG. 17 is a bar chart showing results of an in silico analysis using Cas-OFFinder to determine the predicted off-target sites for the 26 PCSK9-targeting spacers assessed for editing activity, as described in Example 10.

FIG. 18A is a bar chart displaying the level of off-target editing at select off-target sites in HEK293 cells expressing the indicated CasX variants, along with gRNA spacer 6.7 targeting the PCSK9 locus, which was assessed using a CSI-seq assay as described in Example 10. The number of off-target CSI-seq reads for the 10 most frequent off-target (“OT”) sites was normalized relative to the number of on-target CSI-seq reads, unless fewer than 10 sites were identified, such that each bar represents the relative level of editing at a unique off-target site in the genome. Symbols indicate off-target sites conserved across CasX variants for a given spacer.

FIG. 18B is a bar chart displaying the level of off-target editing at select off-target sites in HEK293 cells expressing the indicated CasX variants, along with gRNA spacer 6.8 targeting the PCSK9 locus, which was assessed using a CSI-seq assay as described in Example 10. The number of off-target CSI-seq reads for the 10 most frequent off-target (“OT”) sites was normalized relative to the number of on-target CSI-seq reads, unless fewer than 10 sites were identified, such that each bar represents the relative level of editing at a unique off-target site in the genome. Symbols indicate off-target sites conserved across CasX variants for a given spacer.

FIG. 18C is a bar chart displaying level of off-target editing at select off-target sites in HEK293 cells expressing the indicated CasX variants, along with gRNA spacer 6.74 targeting the PCSK9 locus, which were assessed using a CSI-seq assay as described in Example 10. The number of off-target CSI-seq reads for the 10 most frequent off-target (“OT”) sites was normalized relative to the number of on-target CSI-seq reads, unless fewer than 10 sites were identified, such that each bar represents the relative level of editing at a unique off-target site in the genome. Symbols indicate off-target sites conserved across CasX variants for a given spacer.

FIG. 18D is a bar chart displaying the percentage of reads for the top predicted off-target sites relative to the reads for the on-target site for the indicated CasX variants, along with gRNA spacer 6.162 targeting the PCSK9 locus, which were assessed using a CSI-seq assay in HEK293 cells, as described in Example 10. The, unless fewer than 10 sites were identified, such that each bar represents the relative level of editing at a unique off-target site in the genome. Symbols indicate off-target sites conserved across CasX variants for a given spacer.

FIG. 18E is a bar chart displaying the level of off-target editing at select off-target sites in HEK293 cells expressing the indicated CasX variants, along with gRNA spacer 6.164 targeting the PCSK9 locus, which were assessed using a CSI-seq assay as described in Example 10. The number of off-target CSI-seq reads for the 10 most frequent off-target (“OT”) sites was normalized relative to the number of on-target CSI-seq reads, unless fewer than 10 sites were identified, such that each bar represents the relative level of editing at a unique off-target site in the genome. Symbols indicate off-target sites conserved across CasX variants for a given spacer.

FIG. 19A is a diagram of the secondary structure of guide RNA scaffold 235 (SEQ ID NO: 465), noting the regions with CpG motifs, as described in Example 14. CpG motifs in (1) the pseudoknot stem, (2) the scaffold stem, (3) the extended stem bubble, (4) the extended step, and (5) the extended stem loop are labeled on the structure.

FIG. 19B is a diagram of the CpG-reducing mutations that were introduced into each of the five regions in the coding sequence of the guide RNA scaffold, as described in Example 14. The stem loop with the substitute bubble from gRNA 174 has a sequence of

(SEQ ID NO: 14335)
AGCUCCCUCUUCGGAGGGAGCA.

FIG. 20 provides the results of an editing experiment in which AAV vectors with various CpG-reduced or CpG-depleted guide RNA scaffolds were used to edit the B2M locus in induced neurons, as described in Example 14. The AAV vectors were administered at a multiplicity of infection (MOI) of 4e3. The bars show the mean±the SD of two replicates per sample. “No Tx” indicates a non-transduced control, and “NT” indicates a control with a non-targeting spacer.

FIG. 21 provides the results of an editing experiment in which AAV vectors with various CpG-reduced or CpG-depleted guide RNA scaffolds were used to edit the B2M locus in induced neurons, as described in Example 14. The AAV vectors were administered at an MOI of 3e3. The bars show the mean±the SD of two replicates per sample. “No Tx” indicates a non-transduced control.

FIG. 22 provides the results of an editing experiment in which AAV vectors with various CpG-reduced or CpG-depleted guide RNA scaffolds were used to edit the B2M locus in induced neurons, as described in Example 14. The AAV vectors were administered at an MOI of 1e3. The bars show the mean±the SD of two replicates per sample. “No Tx” indicates a non-transduced control.

FIG. 23 provides the results of an editing experiment in which AAV vectors with various CpG-reduced or CpG-depleted guide RNA scaffolds were used to edit the B2M locus in induced neurons, as described in Example 14. The AAV vectors were administered at an MOI of MOI=3e2. The bars show the mean±the SD of two replicates per sample. “No Tx” indicates a non-transduced control.

FIG. 24A is a bar chart showing the quantification of unnormalized percent editing measured for the indicated targeting spacers and engineered CasX as indel rate detected by NGS at the PCSK9 locus in HEK293T cells at two days post-transfection, as described in Example 14.

FIG. 24B is a bar chart showing the quantification of normalized percent editing measured for the indicated targeting spacers and engineered CasX as indel rate detected by NGS at the PCSK9 locus in HEK293T cells at two days post-transfection, as described in Example 14. Editing rates were normalized by transfection efficiency determined by mScarlet expression for the indicated experimental condition.

FIG. 24C is a bar chart showing the quantification of editing activity measured as indel rate by NGS for the indicated targeting spacers and CasX variants 515, 593, and 812 in a low-dose lentiviral transduction experiment in HEK293T cells, as described in Example 14. A non-targeting (NT) spacer was used as an experimental control.

FIG. 25 is a bar chart showing the quantification of normalized percent editing measured for the indicated targeting spacers as indel rate detected at the PCSK9 locus in HepG2 cells at three days post-transfection with a construct encoding for CasX 515, 593, or 812 and a PCSK9-targeting gRNA, as described in Example 15. A non-targeting spacer (NT) served as a negative experimental control.

FIG. 26A is a bar chart showing the quantification of percent editing measured for the indicated targeting spacers as indel rate detected at the PCSK9 locus in African green monkey (AGM) fibroblasts transduced with AAV particles containing constructs encoding for CasX 491 with the indicated PCSK9-targeting gRNA, as described in Example 16. A non-targeting spacer (NT) served as a negative experimental control.

FIG. 26B is a bar chart showing the quantification of percent editing measured for the indicated targeting spacers as indel rate detected at the PCSK9 locus in cynomolgus macaque (CM) fibroblasts transduced with AAV particles containing constructs encoding for CasX 491 with the indicated PCSK9-targeting gRNA, as described in Example 16. A non-targeting spacer (NT) served as a negative experimental control.

FIG. 27A is a plot showing an inverse correlation between editing at the PCSK9 locus (measured as percent editing quantified as indel rate, as detected by NGS) and secreted PCSK9 levels (measured by ELISA) in primary cynomolgus macaque hepatocytes transduced with lentiviral particles containing a transgene encoding for CasX 515 with the indicated PCSK9-targeting gRNAs, as described in Example 17. A non-targeting (NT) spacer served as a negative experimental control. “L” denotes a low dose of 15 μL, while “H” denotes a high dose of 50 μL.

FIG. 27B is a plot showing an inverse correlation between editing at the PCSK9 locus (measured as percent editing quantified as indel rate as detected by NGS) and secreted PCSK9 levels (measured by ELISA) in primary cynomolgus macaque hepatocytes transduced with lentiviral particles containing a transgene encoding for CasX 812 with the indicated PCSK9-targeting gRNAs, as described in Example 17. A non-targeting (NT) spacer served as a negative experimental control. “L” denotes a low dose of 15 μL, while “H” denotes a high dose of 50 μL.

FIG. 28A is a graph showing PCSK9 secretion levels detected in the media supernatant of cultured primary cynomolgus macaque hepatocytes transduced with lentiviral particles containing a transgene encoding for CasX 515 with a PCSK9-targeting gRNA at the indicated titers, as described in Example 17. A non-targeting (NT) spacer served as a negative experimental control.

FIG. 28B is a graph showing PCSK9 secretion levels detected in the media supernatant of cultured primary cynomolgus macaque hepatocytes transduced with lentiviral particles containing a transgene encoding for CasX 812 with a PCSK9-targeting gRNA at the indicated titers, as described in Example 17. A non-targeting (NT) spacer served as a negative experimental control.

FIG. 29A is a plot showing the correlation of vg/dg (vector genomes per diploid genome) with secreted PCSK9 levels in the media supernatant of cultured primary cynomolgus macaque hepatocytes transduced with lentiviral particles containing a transgene encoding either CasX 515 or CasX 812 with PCSK9-targeting spacer 6.1, as described in Example 17.

FIG. 29B is a plot showing the correlation of vg/dg with secreted PCSK9 levels in the media supernatant of cultured primary cynomolgus macaque hepatocytes transduced with lentiviral particles containing a transgene encoding either CasX 515 or CasX 812 with PCSK9-targeting spacer 6.86, as described in Example 17.

FIG. 29C is a plot showing the correlation of vg/dg with secreted PCSK9 levels in the media supernatant of cultured primary cynomolgus macaque hepatocytes transduced with lentiviral particles containing a transgene encoding either CasX 515 or CasX 812 with PCSK9-targeting spacer 6.109, as described in Example 17.

FIG. 29D is a plot showing the correlation of vg/dg with secreted PCSK9 levels in the media supernatant of cultured primary cynomolgus macaque hepatocytes transduced with lentiviral particles containing a transgene encoding either CasX 515 or CasX 812 with PCSK9-targeting spacer 6.114, as described in Example 17.

FIG. 29E is a plot showing the correlation of vg/dg with secreted PCSK9 levels in the media supernatant of cultured primary cynomolgus macaque hepatocytes transduced with lentiviral particles containing a transgene encoding either CasX 515 or CasX 812 with PCSK9-targeting spacer 6.197, as described in Example 17.

FIG. 29F is a plot showing the correlation of vg/dg with secreted PCSK9 levels in the media supernatant of cultured primary cynomolgus macaque hepatocytes transduced with lentiviral particles containing a transgene encoding either CasX 515 or CasX 812 with PCSK9-targeting spacer 6.203, as described in Example 17.

FIG. 29G is a graph showing the overlay of the data displayed in FIGS. 29A-29F for primary cynomolgus macaque hepatocytes transduced with lentiviral particles containing a transgene encoding CasX 515 with the indicated PCSK9-targeting spacers, as described in Example 17.

FIG. 29H is a graph showing the overlay of the data displayed in FIGS. 29A-29F for primary cynomolgus macaque hepatocytes transduced with lentiviral particles containing a transgene encoding CasX 812 with the indicated PCSK9-targeting spacers, as described in Example 17.

FIG. 30A is a graph showing the correlation of vg/dg with editing activity measured as indel rate detected at the PCSK9 locus in primary cynomolgus macaque hepatocytes transduced with lentiviral particles containing a transgene encoding CasX 515 with the indicated PCSK9-targeting spacers, as described in Example 17.

FIG. 30B is a graph showing the correlation of vg/dg with editing activity measured as indel rate detected at the PCSK9 locus in primary cynomolgus macaque hepatocytes transduced with lentiviral particles containing a transgene encoding CasX 812 with the indicated PCSK9-targeting spacers, as described in Example 17.

FIG. 31 is a bar chart showing the level of off-target editing at all off-target sites normalized relative to the level of on-target editing in HEK293 cells expressing CasX proteins 515, 593, or 812 and for the indicated spacers, as determined using a CSI-seq assay as described in Example 18.

FIG. 32A is a bar plot showing the results of an editing assay, measured as indel rate detected by NGS, at the on-target site (“Target site”) and the four off-target (“OT”) sites nominated by CSI-seq, in HEK293T cells. Cells were harvested two days post-transfection with a lentiviral plasmid encoding for either CasX 515 or 812 with gRNA scaffold 235 and spacer 6.1, as described in Example 18. Indel rate was normalized to transfection efficiency as measured by mScarlet fluorescence.

FIG. 32B is a bar plot showing the results of an editing assay, measured as indel rate detected by NGS, at the on-target site (“Target site”) and the four off-target (“OT”) sites nominated by CSI-seq, in HEK293T cells. Cells were harvested two days post-transfection with a lentiviral plasmid encoding for either CasX 515 or 812 with gRNA scaffold 235 and spacer 6.7, as described in Example 18. Indel rate was normalized to transfection efficiency as measured by mScarlet fluorescence.

FIG. 32C is a bar plot showing the results of an editing assay, measured as indel rate detected by NGS, at the on-target site (“Target site”) and the four off-target (“OT”) sites nominated by CSI-seq, in HEK293T cells. Cells were harvested two days post-transfection with a lentiviral plasmid encoding for either CasX 515 or 812 with gRNA scaffold 235 and spacer 6.8, as described in Example 18. Indel rate was normalized to transfection efficiency as measured by mScarlet fluorescence.

FIG. 33 is a bar graph showing the quantification of percent editing measured as indel rate detected by NGS at the mouse PCSK9 locus in Hepa1-6 cells transfected with the indicated engineered CasX mRNAs and targeting spacers. Cells were harvested at 20 hours post-transfection, as described in Example 19.

FIG. 34 is a violin plot showing the distribution of secreted PCSK9 levels in HepG2 cells transfected with CasX 676 mRNA #2 and a gRNA with the indicated PCSK9-targeting spacer, as described in Example 20. Naïve, untreated cells and cells transfected with CasX 676 mRNA only served as experimental controls.

FIG. 35 is a pair of images of a representative western blot showing the levels of pro-PCSK9 and processed PCSK9 protein (top western blot image) in HepG2 cells transfected with CasX 676 mRNA and a gRNA with the indicated PCSK9-targeting spacer, as described in Example 20. Naïve, untreated cells and cells transfected with CasX 676 mRNA only served as experimental controls. Lysate from HEK293T cells, which do not express the PCSK9 protein, and a cynomolgus macaque recombinant PCSK9 protein control were used as western blot controls. The bottom western blot image shows the total protein loading control.

FIG. 36 is a bar plot showing the western blot quantification for pro-PCSK9, processed PCSK9, and total PCSK9 levels for each of the indicated spacers assessed when transfected with CasX 676 mRNA into HepG2 cells, as described in Example 20. Naïve, untreated cells and cells transfected with CasX 676 mRNA only served as experimental controls. PCSK9 levels were normalized to total PCSK9 levels from the naïve condition.

FIG. 37A is a plot illustrating the percent reduction of secreted PCSK9 level, relative to the non-targeting (NT) control, for primary human hepatocytes from lot #31 treated with the indicated doses of LNPs formulated with CasX 515 or CasX 812 mRNA and a PCSK9-targeting gRNA with spacer 6.1, as described in Example 21.

FIG. 37B is a plot illustrating the percent reduction of secreted PCSK9 level, relative to the non-targeting (NT) control, for primary human hepatocytes from lot #31 treated with the indicated doses of LNPs formulated with CasX 515 or CasX 812 mRNA and a PCSK9-targeting gRNA with spacer 6.8, as described in Example 21.

FIG. 37C is a plot illustrating the percent reduction of secreted PCSK9 level, relative to the non-targeting (NT) control, for primary human hepatocytes from lot #51 treated with the indicated doses of LNPs formulated with CasX 515 or CasX 812 mRNA and a PCSK9-targeting gRNA with spacer 6.1, as described in Example 21.

FIG. 37D is a plot illustrating the percent reduction of secreted PCSK9 level, relative to the non-targeting (NT) control, for primary human hepatocytes from lot #51 treated with the indicated doses of LNPs formulated with CasX 515 or CasX 812 mRNA and a PCSK9-targeting gRNA with spacer 6.8, as described in Example 21.

FIG. 38A is a plot depicting the results of an editing assay, measured as indel rate detected by NGS, at the PCSK9 locus in primary human hepatocytes from lot #31. Hepatocytes were treated with the indicated doses of LNPs formulated with CasX 515 or CasX 812 mRNA and a PCSK9-targeting gRNA with spacer 6.8, as described in Example 21.

FIG. 38B is a plot depicting the results of an editing assay, measured as indel rate detected by NGS, at the PCSK9 locus in primary human hepatocytes from lot #51. Hepatocytes were treated with the indicated doses of LNPs formulated with CasX 515 or CasX 812 mRNA and a PCSK9-targeting gRNA with spacer 6.1, as described in Example 21.

FIG. 38C is a plot depicting the results of an editing assay, measured as indel rate detected by NGS, at the PCSK9 locus in primary human hepatocytes from lot #51. Hepatocytes were treated with the indicated doses of LNPs formulated with CasX 515 or CasX 812 mRNA and a PCSK9-targeting gRNA with spacer 6.8, as described in Example 21.

FIG. 39 depicts a schematic of the relative locations in the human PCSK9 locus targeted by the 122 TTC spacers, as described in Example 22. Spacers are indicated by black vertical bars.

FIG. 40A is a bar plot that shows the results of an editing assay, measured as indel rate detected by NGS, at the PCSK9 locus in HepG2 cells. HepG2 cells were treated with lentiviral particles containing the indicated CasX variants paired with targeting gRNAs containing the indicated spacers, as described in Example 22.

FIG. 40B is a violin plot illustrating a distribution and comparison of total indel rates for the three indicated CasX variants at the PCSK9 locus in HepG2 cells treated with lentiviral particles, as described in Example 22. A two-way ANOVA was performed to evaluate statistical significance: **p<0.01; “N.S” denotes not significant.

### DETAILED DESCRIPTION

While exemplary embodiments have been shown and described herein, it will be obvious to those skilled in the art that such embodiments are provided by way of example only. Numerous variations, changes, and substitutions will now occur to those skilled in the art without departing from the inventions claimed herein. It should be understood that various alternatives to the embodiments described herein may be employed in practicing the embodiments of the disclosure. It is intended that the claims define the scope of the invention and that methods and structures within the scope of these claims and their equivalents be covered thereby.

Unless otherwise defined, all technical and scientific terms used herein have the same meaning as commonly understood by one of ordinary skill in the art to which this invention belongs. Although methods and materials similar or equivalent to those described herein can be used in the practice or testing of the present embodiments, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

“Hybridizable” or “complementary” are used interchangeably to mean that a nucleic acid (e.g., RNA, DNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. It is understood that the sequence of a polynucleotide need not be 100% complementary to that of its target nucleic acid to be specifically hybridizable; it can have at least about 70%, at least about 80%, or at least about 90%, or at least about 95% sequence identity and still hybridize to the target nucleic acid. Moreover, a polynucleotide may hybridize over one or more segments such that intervening or adjacent segments are not involved in the hybridization event (e.g., a loop structure or hairpin structure, a ‘bulge’, ‘bubble’ and the like). Thus, the skilled artisan will understand that while individual bases within a sequence may not be complementary to another sequence, the sequence as a whole is still considered to be complementary.

A “gene,” for the purposes of the present disclosure, includes a DNA region encoding a gene product (e.g., a protein, RNA), as well as all DNA regions which regulate the production of the gene product, whether or not such regulatory sequences are adjacent to coding and/or transcribed sequences. Accordingly, a gene may include accessory element sequences including, but not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Coding sequences encode a gene product upon transcription or transcription and translation; the coding sequences of the disclosure may comprise fragments and need not contain a full-length open reading frame. A gene can include both the strand that is transcribed as well as the complementary strand containing the anticodons.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

The term “adjacent to” with respect to polynucleotide or amino acid sequences refers to sequences that are next to, or adjoining each other in a polynucleotide or polypeptide. The skilled artisan will appreciate that two sequences can be considered to be adjacent to each other and still encompass a limited amount of intervening sequence, e.g., 1, 2, 3, 4, 5, 6, 7, 8, 9 or 10 nucleotides or amino acids.

The term “regulatory element” is used interchangeably herein with the term “regulatory sequence,” and is intended to include promoters, enhancers, and other expression regulatory elements. It will be understood that the choice of the appropriate regulatory element will depend on the encoded component to be expressed (e.g., protein or RNA) or whether the nucleic acid comprises multiple components that require different polymerases or are not intended to be expressed as a fusion protein.

The term “accessory element” is used interchangeably herein with the term “accessory sequence,” and is intended to include, inter alia, polyadenylation signals (poly(A) signal), enhancer elements, introns, posttranscriptional regulatory elements (PTREs, sometimes also referred to as TREs), nuclear localization signals (NLS), deaminases, DNA glycosylase inhibitors, additional promoters, factors that stimulate CRISPR-mediated homology-directed repair (e.g. in cis or in trans), self-cleaving sequences, and fusion domains, for example a fusion domain fused to a CRISPR protein. It will be understood that the choice of the appropriate accessory element or elements will depend on the encoded component to be expressed (e.g., protein or RNA) or whether the nucleic acid comprises multiple components that require different polymerases or are not intended to be expressed as a fusion protein.

The term “promoter” refers to a DNA sequence that contains a transcription start site and additional sequences to facilitate polymerase binding and transcription. Exemplary eukaryotic promoters include elements such as a TATA box, and/or B recognition element (BRE) and assists or promotes the transcription and expression of an associated transcribable polynucleotide sequence and/or gene (or transgene). A promoter can be synthetically produced or can be derived from a known or naturally occurring promoter sequence or another promoter sequence. A promoter can also include a chimeric promoter comprising a combination of two or more heterologous sequences to confer certain properties. A promoter of the present disclosure can include variants of promoter sequences that are similar in composition, but not identical to, other promoter sequence(s) known or provided herein. A promoter can be classified according to criteria relating to the pattern of expression of an associated coding or transcribable sequence or gene operably linked to the promoter, such as constitutive, developmental, tissue-specific, inducible, etc. A promoter can also be classified according to its strength. As used in the context of a promoter, “strength” refers to the rate of transcription of the gene controlled by the promoter. A “strong” promoter means the rate of transcription is high, while a “weak” promoter means the rate of transcription is relatively low.

A promoter of the disclosure can be a Polymerase II (Pol II) promoter. Polymerase II transcribes all protein coding and many non-coding genes. A representative Pol II promoter includes a core promoter, which is a sequence of about 100 base pairs surrounding the transcription start site, and serves as a binding platform for the Pol II polymerase and associated general transcription factors. The promoter may contain one or more core promoter elements such as the TATA box, BRE, Initiator (INR), motif ten element (MTE), downstream core promoter element (DPE), downstream core element (DCE), although core promoters lacking these elements are known in the art. All Pol II promoters are envisaged as within the scope of the instant disclosure.

A promoter of the disclosure can be a Polymerase III (Pol III) promoter. Pol III transcribes DNA to synthesize small ribosomal RNAs such as the 5S rRNA, tRNAs, and other small RNAs. Representative Pol III promoters use internal control sequences (sequences within the transcribed section of the gene) to support transcription, although upstream elements such as the TATA box are also sometimes used. All Pol III promoters are envisaged as within the scope of the instant disclosure.

The term “enhancer” refers to regulatory DNA sequences that, when bound by specific proteins called transcription factors, regulate the expression of an associated gene. Enhancers may be located in the intron of the gene, or 5′ or 3′ of the coding sequence of the gene. Enhancers may be proximal to the gene (i.e., within a few tens or hundreds of base pairs (bp) of the promoter), or may be located distal to the gene (i.e., thousands of bp, hundreds of thousands of bp, or even millions of bp away from the promoter). A single gene may be regulated by more than one enhancer, all of which are envisaged as within the scope of the instant disclosure.

As used herein, a “post-transcriptional regulatory element (PTRE, or TRE),” such as a hepatitis PTRE, refers to a DNA sequence that, when transcribed creates a tertiary structure capable of exhibiting post-transcriptional activity to enhance or promote expression of an associated gene operably linked thereto.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “enhancers” and “promoters”, above).

The term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant polypeptide” or “recombinant protein” refers to a polypeptide or protein which is not naturally occurring, e.g., is made by the artificial combination of two otherwise separated segments of amino sequence through human intervention. Thus, e.g., a protein that comprises a heterologous amino acid sequence is recombinant.

As used herein, “lipoprotein”, such as VLDL, LDL and HDL, refers to a group of proteins found in the serum, plasma and lymph and are important for lipid transport. The chemical composition of each lipoprotein differs, for example, in that the HDL has a higher proportion of protein versus lipid, whereas the VLDL has a lower proportion of protein versus lipid.

As used herein, “atherosclerosis” means a hardening of the arteries affecting large and medium-sized arteries and is characterized by the presence of fatty deposits. The fatty deposits are called “atheromas” or “plaques,” which consist mainly of cholesterol and other fats, calcium and scar tissue, and damage the lining of arteries.

As used herein, “coronary heart disease (CHD)” means a narrowing of the small blood vessels that supply blood and oxygen to the heart, which is often a result of atherosclerosis.

As used herein, “dyslipidemia” refers to a disorder of lipid and/or lipoprotein metabolism, including lipid and/or lipoprotein overproduction or deficiency. Dyslipidemias can be manifested by elevation of lipids such as chylomicron, cholesterol and triglycerides as well as lipoproteins such as low-density lipoprotein (LDL) cholesterol.

As used herein, “high density lipoprotein-C” or “HDL-C” means cholesterol associated with high-density lipoprotein particles. Concentration of HDL-C in serum (or plasma) is typically quantified in mg/dL or nmol/L. “Serum HDL-C” and “plasma HDL-C” mean HDL-C in serum and plasma, respectively.

As used herein, “low density lipoprotein-cholesterol (LDL-C)” means cholesterol carried in low density lipoprotein particles. Concentration of LDL-C in serum (or plasma) is typically quantified in mg/dL or nmol/L. “Serum LDL-C” and “plasma LDL-C” mean LDL-C in the serum and plasma, respectively.

The term “low-density lipoprotein (LDL)” refers to one of the five major groups of lipoprotein, from least dense (lower weight-volume ratio particles) to most dense (larger weight-volume ratio particles): chylomicrons, very low-density lipoproteins (VLDL), low-density lipoproteins (LDL), intermediate-density lipoproteins (IDL), and high-density lipoproteins (HDL). Lipoproteins transfer lipids (fats) around the body in the extracellular fluid thereby facilitating the transfer of fats to the cells body via receptor-mediated endocytosis. An LDL particle is about 220-275 angstroms in diameter.

“Low-density lipoprotein (LDL) receptor” refers to a receptor protein of 839 amino acids (after removal of 21-amino acid signal peptide) that mediates the endocytosis of cholesterol-rich LDL particles. It is a cell-surface receptor that recognizes the apoprotein B100 and apoE protein found in chylomicron remnants and VLDL remnants (IDL) resulting in the binding and endocytosis of LDL-cholesterol. This process occurs in all nucleated cells, but mainly in the liver which removes approximately 70% of LDL from the circulation. The human LDLR gene is described in part in the NCBI database (ncbi.nlm.nih.gov) as Reference Sequence NG_009060.1, which is incorporated by reference herein.

As used herein, “hypercholesterolemia” means a condition characterized by elevated cholesterol or circulating (plasma) cholesterol, LDL-cholesterol and VLDL-cholesterol, as per the guidelines of the Expert Panel Report of the National Cholesterol Educational Program (NCEP) of Detection, Evaluation of Treatment of high cholesterol in adults (see, Arch. Int. Med. 148:36 (1988)).

As used herein, “hyperlipidemia” or “hyperlipemia” is a condition characterized by elevated serum lipids or circulating (plasma) lipids. This condition manifests an abnormally high concentration of fats. The lipid fractions in the circulating blood are cholesterol, low-density lipoproteins, very low density lipoproteins, chylomicrons and triglycerides. The Fredrickson classification of hyperlipidemias is based on the pattern of TG and cholesterol-rich lipoprotein particles, as measured by electrophoresis or ultracentrifugation and is commonly used to characterize primary causes of hyperlipidemias such as hypertriglyceridemia.

As used herein, “triglyceride” or “TG” means a lipid or neutral fat consisting of glycerol combined with three fatty acid molecules.

As used herein, “hypertriglyceridemia” means a condition characterized by elevated triglyceride levels. Its etiology includes primary (i.e. genetic causes) and secondary (other underlying causes such as diabetes, metabolic syndrome/insulin resistance, obesity, physical inactivity, cigarette smoking, excess alcohol and a diet very high in carbohydrates) factors or, most often, a combination of both

As used herein, “diabetes mellitus” or “diabetes” is a syndrome characterized by disordered metabolism and abnormally high blood sugar (hyperglycemia) resulting from insufficient levels of insulin or reduced insulin sensitivity. The characteristic symptoms are excessive urine production (polyuria) due to high blood glucose levels, excessive thirst and increased fluid intake (polydipsia) attempting to compensate for increased urination, blurred vision due to high blood glucose effects on the eye's optics, unexplained weight loss, and lethargy.

As used herein, “diabetic dyslipidemia” or “type 2 diabetes with dyslipidemia” means a condition characterized by Type 2 diabetes, reduced HDL-C, elevated triglycerides (TG), and elevated small, dense LDL particles. As used herein, “lipid nanoparticle” or “LNP” refers to a particle having at least one dimension on the order of nanometers (e.g., 1-1,000 nm) comprising one or more lipids (e.g., cationic lipids, non-cationic lipids, helper phospholipids, and PEG-modified lipids), as well as cholesterol. Specific components of LNP are described more fully, below. In some embodiments, lipid nanoparticles are included in a formulation that can be used to deliver an active agent or therapeutic agent, such as a nucleic acid (e.g., mRNA) to a target site of interest (e.g., cell, tissue, organ, tumor, and the like). In some embodiments, the lipid nanoparticles of the disclosure comprise a nucleic acid. Such lipid nanoparticles typically comprise neutral lipids, charged lipids, steroids and polymer conjugated lipids. In some embodiments, the active agent or therapeutic agent, such as a nucleic acid, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

As used herein, “lipid nanoparticle” refers to a particles having at least one dimension on the order of nanometers (e.g., 1-1,000 nm) comprising one or more lipids (e.g., cationic lipids, non-cationic lipids, and PEG-modified lipids). In some embodiments, lipid nanoparticles are included in a formulation that can be used to deliver an active agent or therapeutic agent, such as a nucleic acid (e.g., mRNA) to a target site of interest (e.g., cell, tissue, organ, tumor, and the like). In some embodiments, the lipid nanoparticles of the disclosure comprise a nucleic acid. Such lipid nanoparticles typically comprise neutral lipids, charged lipids, steroids and polymer conjugated lipids. In some embodiments, the active agent or therapeutic agent, such as a nucleic acid, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

As used herein, “lipid encapsulated” refers to a lipid nanoparticle that provides an active agent or therapeutic agent, such as a nucleic acid (e.g., mRNA), with full encapsulation, partial encapsulation, or both. In an embodiment, the nucleic acid (e.g., mRNA) is fully encapsulated in the lipid nanoparticle.

As used herein, the term “contacting” means establishing a physical connection between two or more entities. For example, contacting a target nucleic acid with a guide nucleic acid means that the target nucleic acid and the guide nucleic acid are made to share a physical connection; e.g., can hybridize if the sequences share sequence similarity.

The disclosure provides systems and methods useful for editing a target nucleic acid sequence. As used herein “editing” is used interchangeably with “modifying” and “modification” and includes but is not limited to cleaving, nicking, deleting, knocking in, knocking out, and the like.

“Dissociation constant”, or “K_{d}”, are used interchangeably and mean the affinity between a ligand “L” and a protein “P”; i.e., how tightly a ligand binds to a particular protein. It can be calculated using the formula K_{d}=[L] [P]/[LP], where [P], [L] and [LP] represent molar concentrations of the protein, ligand and complex, respectively.

The disclosure provides systems and methods useful for editing a target nucleic acid sequence. As used herein “editing” is used interchangeably with “modifying” and “modification” and includes but is not limited to cleaving, nicking, deleting, knocking in, knocking out, and the like.

By “cleavage” it is meant the breakage of the covalent backbone of a target nucleic acid molecule (e.g., RNA, DNA). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events.

The term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. The term “knock-down” as used herein refers to reduction in the expression of a gene or its gene product(s). As a result of a gene knock-down, the protein activity or function may be attenuated or the protein levels may be reduced or eliminated.

As used herein, “homology-directed repair” (HDR) refers to the form of DNA repair that takes place during repair of double-strand breaks in cells. This process requires nucleotide sequence homology, and uses a donor template to repair or knock-out a target DNA, and leads to the transfer of genetic information from the donor to the target. Homology-directed repair can result in an alteration of the sequence of the target sequence by insertion, deletion, or mutation if the donor template differs from the target DNA sequence and part or all of the sequence of the donor template is incorporated into the target DNA.

As used herein, “non-homologous end joining’ (NHEJ) refers to the repair of double-strand breaks in DNA by direct ligation of the break ends to one another without the need for a homologous template (in contrast to homology-directed repair, which requires a homologous sequence to guide repair). NHEJ often results in the insertion or loss (deletion) of nucleotide sequence near the site of the double-strand break.

As used herein “micro-homology mediated end joining” (MMEJ) refers to a mutagenic DSB repair mechanism, which always associates with deletions flanking the break sites without the need for a homologous template (in contrast to homology-directed repair, which requires a homologous sequence to guide repair). MMEJ often results in the loss (deletion) of nucleotide sequence near the site of the double-strand break.

A polynucleotide or polypeptide has a certain percent “sequence similarity” or “sequence identity” to another polynucleotide or polypeptide, meaning that, when aligned, that percentage of bases or amino acids are the same, and in the same relative position, when comparing the two sequences. Sequence similarity (sometimes referred to as percent similarity, percent identity, or homology) can be determined in a number of different manners. To determine sequence similarity, sequences can be aligned using the methods and computer programs that are known in the art, including BLAST, available over the world wide web at ncbi.nlm.nih.gov/BLAST. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence.

A “vector” or “expression vector” is a replicon, such as plasmid, phage, virus, or cosmid, to which another DNA segment, i.e., an expression cassette, may be attached so as to bring about the replication or expression of the attached segment in a cell.

The term “naturally-occurring” or “unmodified” or “wild type” as used herein as applied to a nucleic acid, a polypeptide, a cell, or an organism, refers to a nucleic acid, polypeptide, cell, or organism that is found in nature.

As used herein, a “mutation” refers to an insertion, deletion, substitution, duplication, or inversion of one or more amino acids or nucleotides as compared to a wild-type or reference amino acid sequence or to a wild-type or reference nucleotide sequence.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

A “host cell,” as used herein, denotes a eukaryotic cell, a prokaryotic cell, or a cell from a multicellular organism (e.g., a cell line) cultured as a unicellular entity, which eukaryotic or prokaryotic cells are used as recipients for a nucleic acid (e.g., an AAV vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an AAV vector.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

As used herein, “treatment” or “treating,” are used interchangeably herein and refer to an approach for obtaining beneficial or desired results, including but not limited to a therapeutic benefit and/or a prophylactic benefit. By therapeutic benefit is meant eradication or amelioration of the underlying disorder or disease being treated. A therapeutic benefit can also be achieved with the eradication or amelioration of one or more of the symptoms or an improvement in one or more clinical parameters associated with the underlying disease such that an improvement is observed in the subject, notwithstanding that the subject may still be afflicted with the underlying disorder.

The terms “therapeutically effective amount” and “therapeutically effective dose”, as used herein, refer to an amount of a drug or a biologic, alone or as a part of a composition, that is capable of having any detectable, beneficial effect on any symptom, aspect, measured parameter or characteristics of a disease state or condition when administered in one or repeated doses to a subject such as a human or an experimental animal. Such effect need not be absolute to be beneficial.

As used herein, “administering” means a method of giving a dosage of a compound (e.g., a composition of the disclosure) or a composition (e.g., a pharmaceutical composition) to a subject.

A “subject” is a mammal. Mammals include, but are not limited to, domesticated animals, non-human primates, humans, dogs, rabbits, mice, rats and other rodents.

All publications, patents, and patent applications mentioned in this specification are herein incorporated by reference to the same extent as if each individual publication, patent, or patent application was specifically and individually indicated to be incorporated by reference.

### I. General Methods

The practice of the present invention employs, unless otherwise indicated, conventional techniques of immunology, biochemistry, chemistry, molecular biology, microbiology, cell biology, genomics and recombinant DNA, which can be found in such standard textbooks as Molecular Cloning: A Laboratory Manual, 3rd Ed. (Sambrook et al., Harbor Laboratory Press 2001); Short Protocols in Molecular Biology, 4^{th} Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that endpoints are included and that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

It will be appreciated that certain features of the disclosure, which are, for clarity, described in the context of separate embodiments, may also be provided in combination in a single embodiment. In other cases, various features of the disclosure, which are, for brevity, described in the context of a single embodiment, may also be provided separately or in any suitable sub-combination. It is intended that all combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. Systems for Genetic Editing of PCSK9 Genes

In a first aspect, the present disclosure provides systems comprising a Class 2, Type V CRISPR nuclease protein and one or more guide ribonucleic acids (gRNA), as well as nucleic acids encoding the CRISPR nuclease proteins and gRNA, for use in modifying a PCSK9 gene (referred to herein as the “target nucleic acid”), inclusive of coding and non-coding regions, of a cell. The genome of the cell can have one or more mutations in the PCSK9 gene. As used herein, a “system”, used interchangeably with “composition”, can comprise a Class 2, Type V CRISPR nuclease protein and one or more gRNAs of the disclosure as gene editing pairs, nucleic acids encoding the CRISPR nuclease proteins and gRNA, as well as vectors comprising the nucleic acids or CRISPR nuclease protein and one or more gRNAs the disclosure.

The PCSK9 gene encodes proprotein convertase subtilisin/kexin Type 9 (“PCSK9”), a protein that binds to the receptor for low-density lipoprotein particles (LDL) for transport of LDL into the cell. The PCSK9 gene encompasses the sequence that spans chr1:55,039,476-55,064,853 of the human genome (GRCh38/hg38) (the notation refers to the chromosome 1 (chr1), starting at the 55,039,476 bp to 55,064,853 bp on chromosome 1 (Homo sapiens Updated Annotation Release 109.20190905, GRCh38.p13) (NCBI). The human PCSK9 gene is described in part in the NCBI database (ncbi.nlm.nih.gov) as Reference Sequence NG_009061.1, which is incorporated by reference herein. The PCSK9 locus has 12 exons that produces an mRNA of 3636 bp encoding a 692-amino acid protein that, following its synthesis, undergoes an autocatalytic cleavage reaction that clips off the prodomain, resulting in an activated protein having 540 amino acids. The prodomain remains attached to the catalytic and resistin-like domains, likely because the prodomain serves as a chaperone and facilitates folding and secretion (Seidah, N G et al., Proc Natl Acad Sci USA 100 (3):928 (2003)). The secretory proprotein convertase neural apoptosis-regulated convertase 1 (NARC-1): liver regeneration and neuronal differentiation (Seidah N G, et al.). This protein, also called neural apoptosis regulated convertase, is a serine protease belonging to the protease K subfamily of subtilases.

The human PCSK9 gene (HGNC: 20001) encodes a protein (Q8NBP7) having the sequence

(SEQ ID NO: 543)
MGTVSSRRSWWPLPLLLLLLLLLGPAGARAQEDEDGDYEELVLALRSEEDGLAEAPEHGTTATFHRCAKDPWRL
PGTYVVVLKEETHLSQSERTARRLQAQAARRGYLTKILHVFHGLLPGFLVKMSGDLLELALKLPHVDYIEEDSS
VFAQSIPWNLERITPPRYRADEYQPPDGGSLVEVYLLDTSIQSDHREIEGRVMVTDFENVPEEDGTRFHRQASK
CDSHGTHLAGVVSGRDAGVAKGASMRSLRVLNCQGKGTVSGTLIGLEFIRKSQLVQPVGPLVVLLPLAGGYSRV
LNAACQRLARAGVVLVTAAGNFRDDACLYSPASAPEVITVGATNAQDQPVTLGTLGTNFGRCVDLFAPGEDIIG
ASSDCSTCFVSQSGTSQAAAHVAGIAAMMLSAEPELTLAELRQRLIHFSAKDVINEAWFPEDQRVLTPNLVAAL
PPSTHGAGWQLFCRTVWSAHSGPTRMATAVARCAPDEELLSCSSFSRSGKRRGERMEAQGGKLVCRAHNAFGGE
GVYAIARCCLLPQANCSVHTAPPAEASMGTRVHCHQQGHVLTGCSSHWEVEDLGTHKPPVLRPRGQPNQCVGHR
EASIHASCCHAPGLECKVKEHGIPAPQEQVTVACEEGWTLTGCSALPGTSHVLGAYAVDNTCVVRSRDVSTTGS
TSEGAVTAVAICCRSRHLAQASQELQ

In some embodiments, the disclosure provides systems specifically designed to modify the PCSK9 gene in eukaryotic cells. In some embodiment, the disclosure provides systems specifically designed to modify the PCSK9 gene in eukaryotic cells having a gain of function mutation. In some embodiments, the disclosure provides systems specifically designed to modify the wild-type PCSK9 gene in eukaryotic cells. In some cases, the CRISPR systems are designed to knock-down or knock-out the PCSK9 gene. Generally, any portion of the PCSK9 gene can be targeted using the programable systems and methods provided herein, described more fully, herein.

In some embodiments, the systems of the disclosure comprise a Class 2, Type V nuclease and a corresponding guide ribonucleic acid (gRNA). In some embodiments, the Class 2, Type V nuclease is selected from the group consisting of Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas14, and/or Caso. In some embodiments, the Class 2, Type V nuclease is a CasX nuclease. In some embodiments, the disclosure provides systems comprising one or more engineered CasX proteins and one or more gRNA as a CasX:gRNA system designed to target and edit specific locations in the target nucleic acid sequence of the PCSK9 gene. Each of these components and their use in the editing of the PCSK9 gene is described herein, below.

### III. CasX Proteins for Modifying a Target Nucleic Acid of a PCSK9 Gene

The present disclosure provides CasX proteins that have utility in the modification of a target nucleic acid of a PCSK9 gene in eukaryotic cells. The term “CasX protein”, as used herein, refers to a family of proteins, and encompasses all naturally-occurring CasX proteins (“reference CasX”), as well as engineered CasX proteins with sequence modifications (interchangeably referred to herein as “CasX variants”) possessing one or more improved characteristics relative to a reference CasX protein, described more fully, below.

The CasX proteins employed in the genome modifying systems are Class 2, Type V nucleases. Although members of Class 2, Type V CRISPR-Cas systems have differences, they share some common characteristics that distinguish them from the Cas9 systems. Firstly, the Class 2, Type V nucleases possess a single RNA-guided RuvC domain-containing effector but no HNH domain, and they recognize a TC protospacer adjacent motif (PAM) 5′ upstream to the target region on the non-targeted strand, which is different from Cas9 systems which rely on G-rich PAM at 3′ side of target sequences. Type V nucleases generate staggered double-stranded breaks distal to the PAM sequence, unlike Cas9, which generates a blunt end in the proximal site close to the PAM. In addition, Type V nucleases degrade ssDNA in trans when activated by target dsDNA or ssDNA binding in cis. In some embodiments, the CasX proteins of the embodiments recognize a 5′-TC PAM motif and produce staggered ends cleaved solely by the RuvC domain.

The present disclosure provides highly-modified CasX proteins having multiple mutations relative to one or more reference CasX proteins. Any changes in the amino acid sequence of a reference CasX protein which results in a CasX and that leads to an improved characteristic relative to the reference CasX protein is considered an engineered CasX protein of the disclosure, provided the CasX retains the ability to form an RNP with a gRNA and retains nuclease activity.

CasX proteins of the disclosure comprise the following domains: a non-target strand binding (NTSB) domain, a target strand loading (TSL) domain, a helical I domain, a helical II domain, an oligonucleotide binding domain (OBD), and a RuvC DNA cleavage domain, and, in some cases, domains can be further divided into subdomains, as listed in Tables 2 and 3.

In some embodiments, a CasX protein can bind and/or modify (e.g., catalyze a single strand break (a “nickase”), or catalyze a double strand break) a target nucleic acid at a specific sequence targeted by an associated gRNA, which hybridizes to a sequence within the target nucleic acid sequence. In some embodiments, the CasX comprises a nuclease domain having double-stranded cleavage activity that generates a double-stranded break within 18-26 nucleotides 5′ of a PAM site on the target strand and 10-18 nucleotides 3′ on the non-target strand, resulting in overhangs that can facilitate a higher degree of editing efficiency or insertion of a donor template nucleic acid by HDR or HITI repair mechanisms of the host cell, compared to other CRISPR systems.

a. Reference CasX Proteins

The disclosure provides naturally-occurring CasX proteins (referred to herein as a “reference CasX protein”), which were subsequently modified to create the engineered CasX of the disclosure. For example, reference CasX proteins can be isolated from naturally occurring prokaryotes, such as Deltaproteobacteria, Planctomycetes, or Candidatus Sungbacteria species. A reference CasX protein (interchangeably referred to herein as a reference CasX polypeptide) is a Class 2, Type V CRISPR/Cas endonuclease belonging to the CasX (interchangeably referred to as Cas12e) family of proteins that interacts with a guide RNA to form a ribonucleoprotein (RNP) complex.

In some cases, a reference CasX protein is isolated or derived from Deltaproteobacter having a sequence of:

| (SEQ ID NO: 1) | |
|---|---|
| 1 | MEKRINKIRK KLSADNATKP VSRSGPMKTL LVRVMTDDLK KRLEKRRKKP EVMPQVISNN |
| 61 | AANNLRMLLD DYTKMKEAIL QVYWQEFKDD HVGLMCKFAQ PASKKIDQNK LKPEMDEKGN |
| 121 | LTTAGFACSQ CGQPLFVYKL EQVSEKGKAY TNYFGRCNVA EHEKLILLAQ LKPEKDSDEA |
| 181 | VTYSLGKFGQ RALDFYSIHV TKESTHPVKP LAQIAGNRYA SGPVGKALSD ACMGTIASFL |
| 241 | SKYQDIIIEH QKVVKGNQKR LESLRELAGK ENLEYPSVTL PPQPHTKEGV DAYNEVIARV |
| 301 | RMWVNLNLWQ KLKLSRDDAK PLLRLKGFPS FPVVERRENE VDWWNTINEV KKLIDAKRDM |
| 361 | GRVFWSGVTA EKRNTILEGY NYLPNENDHK KREGSLENPK KPAKRQFGDL LLYLEKKYAG |
| 421 | DWGKVFDEAW ERIDKKIAGL TSHIEREEAR NAEDAQSKAV LTDWLRAKAS FVLERLKEMD |
| 481 | EKEFYACEIQ LQKWYGDLRG NPFAVEAENR VVDISGFSIG SDGHSIQYRN LLAWKYLENG |
| 541 | KREFYLLMNY GKKGRIRFTD GTDIKKSGKW QGLLYGGGKA KVIDLTFDPD DEQLIILPLA |
| 601 | FGTRQGREFI WNDLLSLETG LIKLANGRVI EKTIYNKKIG RDEPALFVAL TFERREVVDP |
| 661 | SNIKPVNLIG VDRGENIPAV IALTDPEGCP LPEFKDSSGG PTDILRIGEG YKEKQRAIQA |
| 721 | AKEVEQRRAG GYSRKFASKS RNLADDMVRN SARDLFYHAV THDAVLVFEN LSRGFGRQGK |
| 781 | RTFMTERQYT KMEDWLTAKL AYEGLTSKTY LSKTLAQYTS KTCSNCGFTI TTADYDGMLV |
| 841 | RLKKTSDGWA TTLNNKELKA EGQITYYNRY KRQTVEKELS AELDRLSEES GNNDISKWTK |
| 901 | GRRDEALFLL KKRFSHRPVQ EQFVCLDCGH EVHADEQAAL NIARSWLFLN SNSTEFKSYK |
| 961 | SGKQPFVGAW QAFYKRRLKE VWKPNA. |

In some cases, a reference CasX protein is isolated or derived from Planctomycetes having a sequence of:

| (SEQ ID NO: 2) | |
|---|---|
| 1 | MQEIKRINKI RRRLVKDSNT KKAGKTGPMK TLLVRVMTPD LRERLENLRK KPENIPQPIS |
| 61 | NTSRANLNKL LTDYTEMKKA ILHVYWEEFQ KDPVGLMSRV AQPAPKNIDQ RKLIPVKDGN |
| 121 | ERLTSSGFAC SQCCQPLYVY KLEQVNDKGK PHTNYFGRCN VSEHERLILL SPHKPEANDE |
| 181 | LVTYSLGKFG QRALDFYSIH VTRESNHPVK PLEQIGGNSC ASGPVGKALS DACMGAVASF |
| 241 | LTKYQDIILE HQKVIKKNEK RLANLKDIAS ANGLAFPKIT LPPQPHTKEG IEAYNNVVAQ |
| 301 | IVIWVNLNLW QKLKIGRDEA KPLQRLKGFP SFPLVERQAN EVDWWDMVCN VKKLINEKKE |
| 361 | DGKVFWQNLA GYKRQEALLP YLSSEEDRKK GKKFARYQFG DLLLHLEKKH GEDWGKVYDE |
| 421 | AWERIDKKVE GLSKHIKLEE ERRSEDAQSK AALTDWLRAK ASFVIEGLKE ADKDEFCRCE |
| 481 | LKLQKWYGDL RGKPFAIEAE NSILDISGFS KQYNCAFIWQ KDGVKKLNLY LIINYFKGGK |
| 541 | LRFKKIKPEA FEANRFYTVI NKKSGEIVPM EVNFNFDDPN LIILPLAFGK RQGREFIWND |
| 601 | LLSLETGSLK LANGRVIEKT LYNRRTRQDE PALFVALTFE RREVLDSSNI KPMNLIGIDR |
| 661 | GENIPAVIAL TDPEGCPLSR FKDSLGNPTH ILRIGESYKE KQRTIQAAKE VEQRRAGGYS |
| 721 | RKYASKAKNL ADDMVRNTAR DLLYYAVTQD AMLIFENLSR GFGRQGKRTF MAERQYTRME |
| 781 | DWLTAKLAYE GLPSKTYLSK TLAQYTSKTC SNCGFTITSA DYDRVLEKLK KTATGWMTTI |
| 841 | NGKELKVEGQ ITYYNRYKRQ NVVKDLSVEL DRLSEESVNN DISSWTKGRS GEALSLLKKR |
| 901 | FSHRPVQEKF VCLNCGFETH ADEQAALNIA RSWLFLRSQE YKKYQTNKTT GNTDKRAFVE |
| 961 | TWQSFYRKKL KEVWKPAV. |

In some cases, a reference CasX protein is isolated or derived from Candidatus Sungbacteria having a sequence of

| (SEQ ID NO: 3) | |
|---|---|
| 1 | MDNANKPSTK SLVNTTRISD HFGVTPGQVT RVFSFGIIPT KRQYAIIERW FAAVEAARER |
| 61 | LYGMLYAHFQ ENPPAYLKEK FSYETFFKGR PVLNGLRDID PTIMTSAVFT ALRHKAEGAM |
| 121 | AAFHTNHRRL FEEARKKMRE YAECLKANEA LLRGAADIDW DKIVNALRTR LNTCLAPEYD |
| 181 | AVIADFGALC AFRALIAETN ALKGAYNHAL NQMLPALVKV DEPEEAEESP RLRFFNGRIN |
| 241 | DLPKFPVAER ETPPDTETII RQLEDMARVI PDTAEILGYI HRIRHKAARR KPGSAVPLPQ |
| 301 | RVALYCAIRM ERNPEEDPST VAGHFLGEID RVCEKRRQGL VRTPFDSQIR ARYMDIISFR |
| 361 | ATLAHPDRWT EIQFLRSNAA SRRVRAETIS APFEGFSWTS NRTNPAPQYG MALAKDANAP |
| 421 | ADAPELCICL SPSSAAFSVR EKGGDLIYMR PTGGRRGKDN PGKEITWVPG SFDEYPASGV |
| 481 | ALKLRLYFGR SQARRMLINK TWGLLSDNPR VFAANAELVG KKRNPQDRWK LFFHMVISGP |
| 541 | PPVEYLDFSS DVRSRARTVI GINRGEVNPL AYAVVSVEDG QVLEEGLLGK KEYIDQLIET |
| 601 | RRRISEYQSR EQTPPRDLRQ RVRHLQDTVL GSARAKIHSL IAFWKGILAI ERLDDQFHGR |
| 661 | EQKIIPKKTY LANKTGFMNA LSFSGAVRVD KKGNPWGGMI EIYPGGISRT CTQCGTVWLA |
| 721 | RRPKNPGHRD AMVVIPDIVD DAAATGFDNV DCDAGTVDYG ELFTLSREWV RLTPRYSRVM |
| 781 | RGTLGDLERA IRQGDDRKSR QMLELALEPQ PQWGQFFCHR CGFNGQSDVL AATNLARRAI |
| 841 | SLIRRLPDTD TPPTP. |

b. Engineered CasX Proteins

The present disclosure provides Class 2, Type V engineered CasX proteins derived from one or more reference CasX proteins for use in the systems, wherein the engineered CasX comprise at least one modification in at least one domain of the reference CasX protein, including the sequences of SEQ ID NOS: 1-3. Any change in amino acid sequence of a reference CasX protein that leads to an improved characteristic of the CasX protein and that retains the ability of the CasX protein to complex with the gRNA and modify the target nucleic acid is considered an engineered CasX protein of the disclosure (sometimes also referred to herein as “variant” CasX proteins). For example, engineered CasX can comprise one or more amino acid substitutions, insertions, deletions, swapped domains from a second CasX, or any combinations thereof, relative to a reference CasX protein sequence. In some embodiments, the disclosure provides Class 2, Type V, engineered CasX proteins wherein the CasX comprises a RuvC cleavage domain, wherein the RuvC cleavage domain comprises the sequence of amino acids 648-812 of SEQ ID NO: 2 with one or more amino acid modifications relative to the RuvC cleavage domain sequence. In some embodiments, the one or more amino acid modifications of the RuvC domain comprise a modification at a position selected from the group consisting of 1658, A708, and P793. In some embodiments, the one or more amino acid modifications comprise one or more substitutions selected from the group consisting of L379R, F399L, 1658V, and A708K, identified in various high-throughput screens to increase the activity of CasX enzyme in E. coli or human cells. For example, the L379R mutation is in the Helical II domain, proximal to the RNA:DNA heteroduplex, likely increases the ability of the enzyme to bind or unwind DNA through nonspecific ionic interactions with the negatively charged DNA backbone. The F399L and I658V substitutions are in the hydrophobic cores of the Helical II domain and RuvC domain, respectively, and likely help to stabilize the protein via better packing. The A708K substitution is in the Bridge Helix and positioned to interact with the 5′ end of the gRNA, potentially leading to better guide binding. In some embodiments, a system of the disclosure comprises a chimeric CasX protein comprising protein domains from two or more different CasX proteins, described more fully, below.

The engineered CasX proteins of the disclosure have one or more improved characteristics compared to a reference CasX from which they were derived. Exemplary improved characteristics of the engineered CasX proteins, relative to reference CasX include, but are not limited to one or more of increased binding affinity to a gRNA, increased binding affinity to the target nucleic acid, improved ability to utilize a greater spectrum of PAM sequences in the editing of the target nucleic acid, improved unwinding of the target nucleic acid, increased editing activity, improved editing efficiency, improved editing specificity for cleavage of the target nucleic acid, improved editing specificity ratio for cleavage of the target nucleic acid, decreased off-target editing or cleavage of the target nucleic acid, increased percentage of a eukaryotic genome that can be edited, increased cleavage activity of the nuclease, increased target strand loading for double strand cleavage, decreased target strand loading for single strand nicking, increased binding of the non-target strand of DNA, improved protein stability, increased solubility, increased protein:gRNA (RNP) complex stability, increased ability to form cleavage-competent RNP, and improved fusion characteristics. In particular, the engineered CasX proteins of the disclosure have an enhanced ability to efficiently edit and/or bind target DNA, when complexed with a guide RNA scaffold as an RNP, utilizing a PAM TC motif, including PAM sequences selected from TTC, ATC, GTC, or CTC, compared to an RNP of a reference CasX protein and a reference gRNA. In the foregoing, the PAM sequence is located at least 1 nucleotide 5′ to the non-target strand of the protospacer having identity with the targeting sequence of the gRNA in an assay system compared to the editing efficiency and/or binding of an RNP comprising the reference CasX protein and reference gRNA in a comparable assay system. In the foregoing embodiments, the one or more of the improved characteristics of the engineered CasX is at least about 1.1 to about 100,000-fold improved relative to the reference CasX protein of SEQ ID NO: 1, SEQ ID NO: 2, or SEQ ID NO: 3, when assayed in a comparable fashion. In other embodiments, the improved characteristics of the engineered CasX is at least about 1.1-fold, at least about 2-fold, at least about 5-fold, at least about 10-fold, at least about 50-fold, at least about 100-fold, at least about 500-fold, at least about 1000-fold, at least about 5000-fold, at least about 10,000-fold, or at least about 100,000-fold compared to the reference CasX protein of SEQ ID NO: 1, SEQ ID NO: 2, or SEQ ID NO: 3.

In some embodiments, an engineered CasX protein comprises a sequence selected from the group consisting of SEQ ID NOS: 4-7, 9-342, 14126-14286, and 14352-14354. In some embodiments, an engineered CasX protein comprises a sequence selected from the group consisting of SEQ ID NOS: 5-7, 9-342, 14126-14286, and 14352-14354. In some embodiments, an engineered CasX protein comprises a sequence selected from the group consisting of SEQ ID NOS: 45-342, 14126-14286, and 14352-14354. In some embodiments, an engineered CasX protein comprises a sequence selected from the group consisting of SEQ ID NOS: 4-7, 25-60, 62-64, 66, 67, 70-95, 100-182, 184, 188-191, 197, 209-229, 230-228, 230-278, 284-297, 299, 302-305, 308, 309, 311-329, 333-342, 14126-14286, and 14352-14354. In some embodiments, an engineered CasX protein comprises a sequence selected from the group consisting of SEQ ID NOS: 14161, 14243, 14257, 14202, 14137, 14135, 14167, 14263, 14257, 14145, 14173, 14261, 14227, 14352, 14197, 14286, 14153, 14239, 14235, 14200, 14187, 14163, 14216, 14229, 14206, 14354, 14280, 14245, 14265, 14191, 14193, 14199, 14210, 14214, 14222, 14256, 14196, 14213, 14129, 14194, and 14188. In some embodiments, an engineered CasX protein comprises a sequence selected from the group consisting of SEQ ID NOS: 14126-14286, and 14352-14354, or a sequence having at least about 70%, at least about 80%, at least about 90%, or at least about 95%, or at least about 95%, or at least about 96%, or at least about 97%, or at least about 98%, or at least about 99% sequence identity thereto. In some embodiments, an engineered CasX protein comprises a sequence selected from the group consisting of SEQ ID NOS: 14126-14286, and 14352-14354. In some embodiments, an engineered CasX protein comprises a sequence selected from the group consisting of SEQ ID NOS: 14126-14286 and 14352-14354. In some embodiments, an engineered CasX comprises a sequence of any one of SEQ ID NOS: 4-7, as set forth in Table 1, or SEQ ID NOS: 14126-14286 or 14352-14354. In some embodiments, an engineered CasX protein consists of a sequence of any one of SEQ ID NOS: 4-7 as set forth in Table 1. In other embodiments, an engineered CasX protein comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical to a sequence selected from the group consisting of SEQ ID NOS: 4-7, 9-342, 14126-14286, and 14352-14354, wherein the engineered CasX protein retains the functional properties of the ability to form an RNP with a gRNA and retains nuclease activity. In other embodiments, an engineered CasX comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical to a sequence selected from the group consisting of SEQ ID NOS: 5-7, 9-342, 14126-14286, and 14352-14354, and comprises a P at position 793 relative to SEQ ID NO: 2, wherein the engineered CasX protein retains the functional properties of the ability to form an RNP with a gRNA and retains nuclease activity. In some embodiments, an engineered CasX comprises a P at position 793 relative to SEQ ID NO: 2. In some embodiments, an engineered CasX protein comprises a sequence of SEQ ID NO: 5. In some embodiments, an engineered CasX protein consists of a sequence of SEQ ID NO: 5.

TABLE 1
Engineered CasX Sequences

| SEQ | Variant | |
|---|---|---|
| ID NO | No. | Amino Acid Sequence |
| 4 | 491 | QEIKRINKIRRRLVKDSNTKKAGKTGPMKTLLVRVMTPDLRERLENLRKKPENIPQPISN |
| | | TSRANLNKLLTDYTEMKKAILHVYWEEFQKDPVGLMSRVAQPASKKIDQNKLKPEMDEKG |
| | | NLTTAGFACSQCGQPLFVYKLEQVSEKGKAYTNYFGRCNVAEHEKLILLAQLKPEKDSDE |
| | | AVTYSLGKFGQRALDFYSIHVTKESTHPVKPLAQIAGNRYASGPVGKALSDACMGTIASF |
| | | LSKYQDIIIEHQKVVKGNQKRLESLRELAGKENLEYPSVTLPPQPHTKEGVDAYNEVIAR |
| | | VRMWVNLNLWQKLKLSRDDAKPLLRLKGFPSFPLVERQANEVDWWDMVCNVKKLINEKKE |
| | | DGKVFWQNLAGYKRQEALRPYLSSEEDRKKGKKFARYQLGDLLLHLEKKHGEDWGKVYDE |
| | | AWERIDKKVEGLSKHIKLEEERRSEDAQSKAALTDWLRAKASFVIEGLKEADKDEFCRCE |
| | | LKLQKWYGDLRGKPFAIEAENSILDISGFSKQYNCAFIWQKDGVKKLNLYLIINYFKGGK |
| | | LRFKKIKPEAFEANRFYTVINKKSGEIVPMEVNENFDDPNLIILPLAFGKRQGREFIWND |
| | | LLSLETGSLKLANGRVIEKTLYNRRTRQDEPALFVALTFERREVLDSSNIKPMNLIGVDR |
| | | GENIPAVIALTDPEGCPLSRFKDSLGNPTHILRIGESYKEKQRTIQAKKEVEQRRAGGYS |
| | | RKYASKAKNLADDMVRNTARDLLYYAVTQDAMLIFENLSRGFGRQGKRTEMAERQYTRME |
| | | DWLTAKLAYEGLSKTYLSKTLAQYTSKTCSNCGFTITSADYDRVLEKLKKTATGWMTTIN |
| | | GKELKVEGQITYYNRYKRQNVVKDLSVELDRLSEESVNNDISSWTKGRSGEALSLLKKRF |
| | | SHRPVQEKFVCLNCGFETHADEQAALNIARSWLFLRSQEYKKYQTNKTTGNTDKRAFVET |
| | | WQSFYRKKLKEVWKPAV |
| 5 | 515 | QEIKRINKIRRRLVKDSNTKKAGKTGPMKTLLVRVMTPDLRERLENLRKKPENIPQPISN |
| | | TSRANLNKLLTDYTEMKKAILHVYWEEFQKDPVGLMSRVAQPASKKIDQNKLKPEMDEKG |
| | | NLTTAGFACSQCGQPLFVYKLEQVSEKGKAYTNYFGRCNVAEHEKLILLAQLKPEKDSDE |
| | | AVTYSLGKFGQRALDFYSIHVTKESTHPVKPLAQIAGNRYASGPVGKALSDACMGTIASF |
| | | LSKYQDIIIEHQKVVKGNQKRLESLRELAGKENLEYPSVTLPPQPHTKEGVDAYNEVIAR |
| | | VRMWVNLNLWQKLKLSRDDAKPLLRLKGFPSFPLVERQANEVDWWDMVCNVKKLINEKKE |
| | | DGKVFWQNLAGYKRQEALRPYLSSEEDRKKGKKFARYQLGDLLLHLEKKHGEDWGKVYDE |
| | | AWERIDKKVEGLSKHIKLEEERRSEDAQSKAALTDWLRAKASFVIEGLKEADKDEFCRCE |
| | | LKLQKWYGDLRGKPFAIEAENSILDISGFSKQYNCAFIWQKDGVKKLNLYLIINYFKGGK |
| | | LRFKKIKPEAFEANRFYTVINKKSGEIVPMEVNENFDDPNLIILPLAFGKRQGREFIWND |
| | | LLSLETGSLKLANGRVIEKTLYNRRTRQDEPALFVALTFERREVLDSSNIKPMNLIGVDR |
| | | GENIPAVIALTDPEGCPLSRFKDSLGNPTHILRIGESYKEKQRTIQAKKEVEQRRAGGYS |
| | | RKYASKAKNLADDMVRNTARDLLYYAVTQDAMLIFENLSRGFGRQGKRTEMAERQYTRME |
| | | DWLTAKLAYEGLPSKTYLSKTLAQYTSKTCSNCGFTITSADYDRVLEKLKKTATGWMTTI |
| | | NGKELKVEGQITYYNRYKRQNVVKDLSVELDRLSEESVNNDISSWTKGRSGEALSLLKKR |
| | | FSHRPVQEKFVCLNCGFETHADEQAALNIARSWLFLRSQEYKKYQTNKTTGNTDKRAFVE |
| | | TWQSFYRKKLKEVWKPAV |
| 6 | 676 | QEIKRINKIRRRLVKDSNTKKAGKTRGPMKTLLVRVMTPDLRERLENLRKKPENIPQPIS |
| | | NTSRANLNKLLTDYTEMKKAILHVYWEEFQKDPVGLMSRVAQPASKKIDQNKLKPEMDEK |
| | | GNLTTAGFACSQCGQPLFVYKLEQVSEKGKAYTNYFGRCNVAEHEKLIKLAQLKPEKDSD |
| | | EAVTYSLGKFGQRALDFYSIHVTKESTHPVKPLAQIAGNRYASSPVGKALSDACMGTIAS |
| | | FLSKYQDIIIEHQKVVKGNQKRLESLRELAGKENLEYPSVTLPPQPHTKEGVDAYNEVIA |
| | | RVRMWVNLNLWQKLKLSRDDAKPLLRLKGFPSFPLVERQANEVDWWDMVCNVKKLINEKK |
| | | EDGKVFWQNLAGYKRQEALRPYLSSEEDRKKGKKFARYQLGDLLLHLEKKHGEDWGKVYD |
| | | EAWERIDKKVEGLSKHIKLEEERRSEDAQSKAALTDWLRAKASFVIEGLKEADKDEFCRC |
| | | ELKLQKWYGDLRGKPFAIEAENSILDISGFSKQYNCAFIWQKDGVKKLNLYLIINYFKGG |
| | | KLRFKKIKPEAFEANRFYTVINKKSGEIVPMEVNENFDDPNLIILPLAFGKRQGREFIWN |
| | | DLLSLETGSLKLANGRVIEKTLYNRRTRQDEPALFVALTFERREVLDSSNIKPMNLIGVD |
| | | RGENIPAVIALTDPEGCPLSRFKDSLGNPTHILRIGESYKEKQRTIQAKKEVEQRRAGGY |
| | | SRKYASKAKNLADDMVRNTARDLLYYAVTQDAMLIFENLSRGFGRQGKRTEMAERQYTRM |
| | | EDWLTAKLAYEGLPSKTYLSKTLAQYTSKTCSNCGFTITSADYDRVLEKLKKTATGWMTT |
| | | INGKELKVEGQITYYNRYKRQNVVKDLSVELDRLSEESVNNDISSWTKGRSGEALSLLKK |
| | | RFSHRPVQEKFVCLNCGFETHADEQAALNIARSWLFLRSQEYKKYQTNKTTGNTDKRAFV |
| | | ETWQSFYRKKLKEVWKPAV |
| 7 | 812 | QEIKRINKIRRRLVKDSNTKKAGKTGPMKTLLVRVMTPDLRERLENLRKKPENIPQPISN |
| | | TSRANLNKLLTDYTEMKKAILHVYWEEFQKDPVGLMSRVAQPASKKIDQNKLKPEMDEKG |
| | | NLTTAGFACSQCGQPLFVYKLEQVSEKGKAYTNYFGRCNVAEHEKLILLAQLKPEKDSDE |
| | | AVTYSLGKFGQRALDFYSIHVTKESTHPVKPLAQIAGNRYASGPVGKALSDACMGTIASF |
| | | LSKYQDIIIEHQKVVKGNQKRLESLRELAGKENLEYPSVTLPPQPHTKEGVDAYNEVIAR |
| | | VRMWVNLNLWQKLKLSRDDAKPLLRLKKFPSFPLVERQANEVDWWDMVCNVKKLINEKKE |
| | | DGKVFWQNLAGYKRQEALRPYLSSEEDRKKGKKFARYQLGDLLLHLEKKHGEDWGKVYDE |
| | | AWERIDKKVEGLSKHIKLEEERRSEDAQSKAALTDWLRAKASFVIEGLKEADKDEFCRCE |
| | | LKLQKWYGDLRGKPFAIEAENSILDISGFSKQYNCAFIWQKDGVKKLNLYLIINYFKGGK |
| | | LRFKKIKPEAFEANRFYTVINKKSGEIVPMEVNENFDDPNLIILPLAFGKRQGREFIWND |
| | | LLSLETGSLKLANGRVIEKTLYNRRTRQDEPALFVALTFERREVLDSSNIKPMNLIGVDR |
| | | GENIPAVIALTDPEGCPLSRFKDSLGNPTHILRIGESYKEKQRTIQAKKEVEQRRAGGYS |
| | | RKYASKAKNLADDMVRNTARDLLYYAVTQDAMLIFENLSRGFGRQGKRTEMAERQYTRME |
| | | DWLTAKLAYEGLPSKTYLSKTLAQYTSKTCSNCGFTITSADYDRVLEKLKKTATGWMTTI |
| | | NGKELKVEGQITYYNRYKRQNVVKDLSVELDRLSEESVNNDISSWTKGRSGEALSLLKKR |
| | | FSHRPVQEKFVCLNCGFETHADEQAALNIARSWLFLRSQEYKKYQTNKTTGNTDKRAFVE |
| | | TWQSFYRKKLKEVWKPAV |

Further engineered CasX contemplated for use in the systems of the disclosure are described in International Publication Nos. WO2020247882 and WO2022120095, which are hereby incorporated by reference in their entirety.

c. CasX Proteins with Domains from Multiple Source Proteins

Also contemplated within the scope of the disclosure are chimeric CasX proteins. As used herein, a “chimeric CasX” protein refers to both a CasX protein containing at least two domains from different sources, as well a CasX protein containing at least one domain that itself is chimeric. Accordingly, in some embodiments, a chimeric CasX protein is one that includes at least two domains isolated or derived from different sources, such as from two different naturally occurring CasX proteins, (e.g., from two different CasX reference proteins), or from two different engineered CasX proteins. In other embodiments, the chimeric CasX protein is one that contains at least one domain that is a chimeric domain, e.g., in some embodiments, part of a domain comprises a substitution from a different CasX protein (from a reference CasX protein, or another engineered CasX protein).

In some embodiments, the helical I-I domain (sometimes referred to as helical I-a) of the CasX variant derived from SEQ ID NO: 2 is replaced with the corresponding helical I-I sequence from SEQ ID NO: 1, resulting in a chimeric CasX protein.

In some embodiments, the helical I-I domain and NTSB domain of the CasX variant derived from SEQ ID NO: 2 is replaced with the corresponding helical I-I and NTSB sequences from SEQ ID NO: 1, resulting in a chimeric CasX protein.

In some embodiments, an engineered CasX protein is a chimeric CasX protein, and comprises at least one chimeric domain. In some embodiments, the at least one chimeric domain can be any of the NTSB, TSL, helical I, helical II, OBD or RuvC domains as described herein. In the case of split or non-contiguous domains such as helical I, RuvC and OBD, a portion of the non-contiguous domain can be replaced with the corresponding portion from any other source.

Domain sequences from reference CasX proteins, and their coordinates, are shown in Table 2. In some embodiments, the chimeric RuvC domain of an engineered CasX comprises amino acids 660 to 823 of SEQ ID NO: 1 and amino acids 921 to 978 of SEQ ID NO: 2. As an alternative embodiment of the foregoing, a chimeric RuvC domain comprises amino acids 647 to 810 of SEQ ID NO: 2 and amino acids 935 to 986 of SEQ ID NO: 1. In some embodiments, the engineered CasX 472-483, 485-491, 515, 676, and 812 have a NTSB and a portion of the helical I-II domain derived from the reference CasX sequence of SEQ ID NO: 1, while the other domains are derived from the reference CasX sequence of SEQ ID NO: 2, it being understood that the engineered variants have additional amino acid changes at select locations (relative to the reference sequence), and the resulting chimeric CasX proteins were determined to have improved characteristics relative to the reference CasX proteins. In a particular embodiment, the chimeric helical I domain of the chimeric CasX proteins comprises amino acids 59-102 of SEQ ID NO: 2, or a sequence having at least about 70%, at least about 80%, at least about 90%, at least about 95%, or at least about 99% sequence identity thereto (helical I-I), and comprises amino acids 192-332 of SEQ ID NO: 1, or at least about 70%, at least about 80%, at least about 90%, at least about 95%, or at least about 99% sequence identity thereto (helical I-II). In some embodiments, a chimeric CasX protein is selected from the group consisting of SEQ ID NOS: 4-7, 25-60, 62-64, 66, 67, 70-95, 100-182, 184, 188-191, 197, 209-229, 230-228, 230-278, 284-297, 299, 302-305, 308, 309, 311-329, 333-342, 14126-14286, and 14352-14354, or a sequence having at least about 80%, at least about 90%, or at least about 95%, or at least about 95%, or at least about 96%, or at least about 97%, or at least about 98%, or at least about 99% sequence identity thereto. In some embodiments, a chimeric CasX protein is selected from the group consisting of SEQ ID NOS: 45-60, 62-64, 66, 67, 70-95, 100-182, 184, 188-191, 197, 209-229, 230-228, 230-278, 284-297, 299, 302-305, 308, 309, 311-329, 333-342, 14126-14286, and 14352-14354. The skilled artisan will understand that the domain boundaries indicated in Table 1 below are approximate, and that protein fragments whose boundaries differ from those given in the table below by 1, 2, or 3 amino acids may have the same activity as the domains described below.

TABLE 2
Domain coordinates in Reference CasX proteins

| | Coordinates in | Coordinates in |
|---|---|---|
| Domain Name | SEQ ID NO: 1 | SEQ ID NO: 2 |
| OBD-I | 1-55 | 1-57 |
| helical I-I | 56-99 | 58-101 |
| NTSB | 100-190 | 102-191 |
| helical I-II | 191-331 | 192-332 |
| helical II | 332-508 | 333-500 |
| OBD-II | 509-659 | 501-646 |
| RuvC-I | 660-823 | 647-810 |
| TSL | 824-933 | 811-920 |
| RuvC-II | 934-986 | 921-978 |

TABLE 3
Exemplary Domain Sequences in Reference CasX proteins
Deltaproteobacter sp. (reference CasX of SEQ ID NO: 1)

| SEQ | | |
|---|---|---|
| ID | Domain | Sequence |
| 343 | OBD-I | EKRINKIRKKLSADNATKPVSRSGPMKTLLVRVMTDDLKKRLEKRRKKPEVMPQ |
| 344 | helical I-I | VISNNAANNLRMLLDDYTKMKEAILQVYWQEFKDDHVGLMCKFA |
| 345 | NTSB | QPASKKIDQNKLKPEMDEKGNLTTAGFACSQCGQPLFVYKLEQVSEKGKAYTNYFGRCNV |
| | | AEHEKLILLAQLKPEKDSDEAVTYSLGKFGQ |
| 346 | helical I-II | RALDFYSIHVTKESTHPVKPLAQIAGNRYASGPVGKALSDACMGTIASFLSKYQDIIIEH |
| | | QKVVKGNQKRLESLRELAGKENLEYPSVTLPPQPHTKEGVDAYNEVIARVRMWVNLNLWQ |
| | | KLKLSRDDAKPLLRLKGFPSF |
| 347 | helical II | PVVERRENEVDWWNTINEVKKLIDAKRDMGRVFWSGVTAEKRNTILEGYNYLPNENDHKK |
| | | REGSLENPKKPAKRQFGDLLLYLEKKYAGDWGKVFDEAWERIDKKIAGLTSHIEREEARN |
| | | AEDAQSKAVLTDWLRAKASFVLERLKEMDEKEFYACEIQLQKWYGDLRG NPFAVEAE |
| 348 | OBD-II | NRVVDISGESIGSDGHSIQYRNLLAWKYLENGKREFYLLMNYGKKGRIRFTDGTDIKKSG |
| | | KWQGLLYGGGKAKVIDLTFDPDDEQLIILPLAFGTRQGREFIWNDLLSLETGLIKLANGR |
| | | VIEKTIYNKKIG RDEPALFVALTFERREVVD |
| 349 | RuvC-I | PSNIKPVNLIGVDRGENIPAVIALTDPEGCPLPEFKDSSGGPTDILRIGEGYKEKRAIQ |
| | | AAKEVEQRRAGGYSRKFASKSRNLADDMVRNSARDLFYHAVTHDAVLVFENLSRGFGRQG |
| | | KRTFMTERQYTKMEDWLTAKLAYEGLTSKTYLSKTLAQYTSKTC |
| 350 | TSL | SNCGFTITTADYDGMLVRLKKTSDGWATTLNNKELKAEGQITYYNRYKRTVEKELSAEL |
| | | DRLSEESGNNDISKWTKGRRDEALFLLKKRFSHRPVQEQFVCLDCGHEVH |
| 351 | RuvC-II | ADEQAALNIARSWLFLN SNSTEFKSYKSGKQPFVGAWQAFYKRRLKEVWKPNA |

Planctomycetes sp. (Reference CasX of SEQ ID NO: 2)

| SEQ | | |
|---|---|---|
| ID | Domain | Sequence |
| 352 | OBD-I | QEIKRINKIRRRLVKDSNTKKAGKTGPMKTLLVRVMTPDLRERLENLRKKPENIPQ |
| 353 | helical I-I | PISNTSRANLNKLLTDYTEMKKAILHVYWEEFQKDPVGLMSRVA |
| 354 | NTSB | QPAPKNIDRKLIPVKDGNERLTSSGFACSQCCQPLYVYKLEQVNDKGKPHTNYFGRCNV |
| | | SEHERLILLSPHKPEANDELVTYSLGKFGQ |
| 355 | helical I-II | RALDFYSIHVTRESNHPVKPLEQIGGNSCASGPVGKALSDACMGAVASFLTKYQDIILEH |
| | | QKVIKKNEKRLANLKDIASANGLAFPKITLPPPHTKEGIEAYNNVVAQIVIWVNLNLWQ |
| | | KLKIGRDEAKPLQRLKGFPSF |
| 356 | helical II | PLVERQANEVDWWDMVCNVKKLINEKKEDGKVFWQNLAGYKRQEALLPYLSSEEDRKKGK |
| | | KFARYQFGDLLLHLEKKHGEDWGKVYDEAWERIDKKVEGLSKHIKLEEERRSEDAQSKAA |
| | | LTDWLRAKASFVIEGLKEADKDEFCRCELKLKWYGDLRGKPFAIEAE |
| 357 | OBD-II | NSILDISGFSKQYNCAFIWQKDGVKKLNLYLIINYFKGGKLRFKKIKPEAFEANRFYTVI |
| | | NKKSGEIVPMEVNENFDDPNLIILPLAFGKRGREFIWNDLLSLETGSLK |
| | | LANGRVIEKTLYNRRTRQDEPALFVALTFERREVLD |
| 358 | RuvC-I | SSNIKPMNLIGIDRGENIPAVIALTDPEGCPLSRFKDSLGNPTHILRIGESYKEKRTIQ |
| | | AAKEVEQRRAGGYSRKYASKAKNLADDMVRNTARDLLYYAVTQDAMLIFENLSRGFGRQG |
| | | KRTFMAERQYTRMEDWLTAKLAYEGLPSKTYLSKTLAQYTSKTC |
| 359 | TSL | SNCGFTITSADYDRVLEKLKKTATGWMTTINGKELKVEGQITYYNRYKRNVVKDLSVEL |
| | | DRLSEESVNNDISSWTKGRSGEALSLLKKRFSHRPVQEKFVCLNCGFETH |
| 360 | RuvC-II | ADEQAALNIARSWLFLRSQEYKKYQTNKTTGNTDKRAFVETWQSFYRKKLKEVWKPAV |

d. Engineered CasX Derived from Other CasX Variants

In some cases, an engineered CasX of the disclosure is generated by one or more modifications to a previous CasX variant (i.e., by iterating modifications). In further iterations, a variant protein is utilized to generate additional engineered CasX of the disclosure. For example, CasX 119 (SEQ ID NO: 375), CasX 491 (SEQ ID NO: 429), and CasX 515 (SEQ ID NO: 436) are exemplary variant proteins that are modified to generate additional engineered CasXs of the disclosure having improvements or additional properties relative to a reference CasX or the CasX variant from which they were derived. CasX 119 contains a substitution of L379R, a substitution of A708K and a deletion of P at position 793 of SEQ ID NO: 2. CasX 491 contains an NTSB and Helical 1B domain swap from SEQ ID NO: 1. CasX 515 (SEQ ID NO: 5) was derived from CasX 491 by insertion of P at position 793 (relative to SEQ ID NO: 2) and was used to create the CasX variants described in Example 1. For example, CasX 668 (SEQ ID NO: 179) has an insertion of R at position 26 and a substitution of G223S relative to CasX 515. CasX 672 (SEQ ID NO: 183) has substitutions of L169K and G223S relative to CasX 515. CasX 676 (SEQ ID NO: 6) has substitutions of L169K and G223S and an insertion of R at position 26 relative to CasX 515.

Exemplary methods used to generate and evaluate engineered CasXs derived from other CasX variants are described in the Examples, which were created by introducing modifications to the encoding sequence resulting in amino acid substitutions, deletions, or insertions at one or more positions in one or more domains of the CasX variant. In particular, Example 7 describes the methods used to create variants of CasX 515 (SEQ ID NO: 5) that were then assayed to determine those positions in the sequence that, when modified by an amino acid insertion, deletion, or substitution, resulted in an enrichment or improvement in the assays. For purposes of the disclosure, the sequences of the domains of CasX 515 are provided in Table 4 and include an OBD-I domain having the sequence of SEQ ID NO: 352, an OBD-II domain having the sequence of SEQ ID NO: 357, NTSB domain having the sequence of SEQ ID NO: 345, a helical I-I domain having the sequence of SEQ ID NO: 353, a helical I-II domain having the sequence of SEQ ID NO: 14332, a helical II domain having the sequence of SEQ ID NO: 14333, a RuvC-I domain having the sequence of SEQ ID NO: 14334, a RuvC-II domain having the sequence of SEQ ID NO: 360, and a TSL domain having the sequence of SEQ ID NO: 359. By the methods of the disclosure, individual positions in the domains of CasX 515 were modified, assayed, and the resulting positions and exemplary modifications leading to an enrichment or improvement that follow are provided, relative to their position in each domain or subdomain. In some cases, such positions are disclosed in Tables 50 and 52-54 of the Examples. In some embodiments, the disclosure provides engineered CasX derived from CasX 515 comprising one or more modifications (i.e., an insertion, a deletion, or a substitution) at one or more amino acid positions in one or more domains. In a particular approach, as detailed in Examples 26 and 27, single mutations of CasX 515 that demonstrated enhanced editing activity and/or specificity, were selected based on locations deemed to be potentially complementary, and paired to make additional variants that there then screened for activity and specificity.

In some embodiments, an engineered CasX protein comprises a sequence selected from the group consisting of SEQ ID NOS: 14126-14286, and 14352-14354, wherein the engineered CasX protein comprises two or more modifications relative to the CasX 515 protein of SEQ ID NO: 5, and wherein the two or more modifications act to increase activity, specificity, or both, of the engineered CasX protein. In some embodiments, the two or more mutations act additively or synergistically. In some embodiments, the engineered CasX protein comprising a sequence selected from the group consisting of SEQ ID NOS: 14126-14286, and 14352-14354 exhibits greater editing activity, editing specificity, specificity ratio, or a combination thereof, compared to CasX 515 when assayed under equivalent conditions. In some embodiments, the improved characteristics is determined compared to the unmodified parental CasX 515 in an in vitro assay under comparable conditions. In some embodiments, the engineered CasX protein comprises a P at position 793 (corresponding to SEQ ID NO: 2).

In some embodiments, the engineered CasX of SEQ ID NOS: 14126-14286 and 14352-14354 exhibit enhanced editing activity compared to the parental CasX 515. In some embodiments, the engineered CasX exhibiting enhanced editing activity compared to the parental CasX 515 are selected from the group consisting of SEQ ID NOS: 14161, 14243, 14257, 14202, 14137, 14135, 14167, 14263, 14257, 14145, 14173, 14261, 14227, 14352, 14197, 14286, 14153, 14239, 14235, 14200, 14187, 14163, 14216, 14229, 14206, 14354, 14280, 14245, 14265, 14191, 14193, 14199, 14210, 14214, 14222, 14256, 14196, 14213, 14129, 14194, and 14188. In some embodiments, the improved characteristics is determined compared to the unmodified parental CasX 515 in an in vitro assay under comparable conditions.

In some embodiments, the engineered CasX of SEQ ID NOS: 14126-14286 and 14352-14354 exhibit improved editing specificity compared to the parental CasX 515. In some embodiments, the engineered CasX exhibiting improved editing specificity compared to the parental CasX 515 are selected from the group consisting of SEQ ID NOS: 14212, 14182, 14184, 14178, 14180, 14176, 14236, 14273, 14208, 14353, 14224, 14240, 14129, 14132, 14230, 14210, 14161, 14223, 14222, 14177, 14185, 14190, 14266, 14260, 14286, 14134, 14218, 14203, 14194, 14196, 14280, 14156, 14354, 14214, 14232, 14253, 14256, 14188, 14181, 14199, 14187, 14192, 14225, 14130, 14213, 14153, 14237, 14263, 14265, 14191, 14216, 14261, and 14231. In some embodiments, the improved characteristics is determined compared to the unmodified parental CasX 515 in an in vitro assay under comparable conditions.

In some embodiments, the engineered CasX of SEQ ID NOS: 14126-14286 and 14352-14354 exhibit enhanced specificity ratio compared to the parental CasX 515. In some embodiments, the engineered CasX exhibiting improved editing specificity ratio compared to the parental CasX 515 are selected from the group consisting of SEQ ID NOS: 14161, 14176, 14178, 14182, 14184, 14208, 14273, 14212, 14129, 14210, 14180, 14222, 14286, 14353, 14177, 14224, 14185, 14240, 14280, 14132, 14236, 14354, 14263, 14196, 14223, 14194, 14266, 14187, 14260, 14230, 14214, 14153, 14256, 14190, 14156, 14199, 14188, 14253, 14261, 14134, 14216, 14265, 14203, 14218, 14191, 14213, 14257, 14137, 14235, 14232, 14130, 14227, 14239, 14192, 14237, 14225, and 14181. In some embodiments, the improved characteristics is determined compared to the unmodified parental CasX 515 in an in vitro assay under comparable conditions.

In some embodiments, the engineered CasX of SEQ ID NOS: 14126-14286 and 14352-14354 exhibit enhanced editing activity and improved specificity compared to the parental CasX 515. In some embodiments, the engineered CasX of SEQ ID NOS: 14126-14286 and 14352-14354 exhibit enhanced editing activity and specificity ratio compared to the parental CasX 515. In some embodiments, the engineered CasX exhibiting enhanced editing activity and improved editing specificity compared to the parental CasX 515 are selected from the group consisting of SEQ ID NOS: 14161, 14263, 14261, 14286, 14153, 14187, 14216, 14354, 14280, 14265, 14191, 14199, 14210, 14214, 14222, 14256, 14196, 14213, 14129, 14194, and 14188. In some embodiments, the improved characteristics is determined compared to the unmodified parental CasX 515 in an in vitro assay under comparable conditions.

In some embodiments, the engineered CasX of SEQ ID NOS: 14126-14286 and 14352-14354 exhibit enhanced editing activity and improved specificity ratio compared to the parental CasX 515. In some embodiments, the engineered CasX exhibiting enhanced editing activity and improved editing specificity ratio compared to the parental CasX 515 are selected from the group consisting of SEQ ID NOS: 14161, 14257, 14137, 14263, 14257, 14261, 14227, 14286, 14153, 14239, 14235, 14187, 14216, 14354, 14280, 14265, 14191, 14199, 14210, 14214, 14222, 14256, 14196, 14213, 14129, 14194, and 14188. In some embodiments, the improved characteristics is determined compared to the unmodified parental CasX 515 in an in vitro assay under comparable conditions.

TABLE 4
CasX 515 domain sequences

| Domain | SEQ ID NO | Amino Acid Sequence |
|---|---|---|
| OBD-I | 352 | QEIKRINKIRRRLVKDSNTKKAGKTGPMKTLLVRVMTPDLRERLENLRKKPENI |
| | | PQ |
| Helical I-I | 353 | PISNTSRANLNKLLTDYTEMKKAILHVYWEEFQKDPVGLMSRVA |
| NTSB | 345 | QPASKKIDQNKLKPEMDEKGNLTTAGFACSQCGQPLFVYKLEQVSEKGKAYTNY |
| | | FGRCNVAEHEKLILLAQLKPEKDSDEAVTYSLGKFGQ |
| Helical I-II | 14332 | RALDFYSIHVTKESTHPVKPLAQIAGNRYASGPVGKALSDACMGTIASFLSKYQ |
| | | DIIIEHQKVVKGNOKRLESLRELAGKENLEYPSVTLPPQPHTKEGVDAYNEVIA |
| | | RVRMWVNLNLWQKLKLSRDDAKPLLRLKGFPSF |
| Helical II | 14333 | PLVERQANEVDWWDMVCNVKKLINEKKEDGKVFWQNLAGYKRQEALRPYLSSEE |
| | | DRKKGKKFARYQLGDLLLHLEKKHGEDWGKVYDEAWERIDKKVEGLSKHIKLEE |
| | | ERRSEDAQSKAALTDWLRAKASFVIEGLKEADKDEFCRCELKLQKWYGDLRGKP |
| | | FAIEAE |
| OBD-II | 357 | NSILDISGFSKQYNCAFIWQKDGVKKLNLYLIINYFKGGKLRFKKIKPEAFEAN |
| | | RFYTVINKKSGEIVPMEVNENFDDPNLIILPLAFGKROGREFIWNDLLSLETGS |
| | | LKLANGRVIEKTLYNRRTRODEPALFVALTFERREVLD |
| RuvC-I | 14334 | SSNIKPMNLIGVDRGENIPAVIALTDPEGCPLSRFKDSLGNPTHILRIGESYKE |
| | | KORTIQAKKEVEQRRAGGYSRKYASKAKNLADDMVRNTARDLLYYAVTQDAMLI |
| | | FENLSRGFGRQGKRTFMAERQYTRMEDWLTAKLAYEGLPSKTYLSKTLAQYTSK |
| | | TC |
| TSL | 359 | SNCGFTITSADYDRVLEKLKKTATGWMTTINGKELKVEGQITYYNRYKRQNVVK |
| | | DLSVELDRLSEESVNNDISSWTKGRSGEALSLLKKRFSHRPVQEKFVCLNCGFE |
| | | TH |
| RuvC-II | 360 | ADEQAALNIARSWLFLRSQEYKKYQTNKTTGNTDKRAFVETWQSFYRKKLKEVW |
| | | KPAV |

e. CasX Fusion Proteins

Also contemplated within the scope of the disclosure are engineered CasX proteins comprising a heterologous protein fused to the CasX for use in the systems of the disclosure. This includes engineered CasX comprising N-terminal or C-terminal fusions of the CasX to a heterologous protein or domain thereof. In some embodiments, the engineered CasX protein is fused to one or more proteins or domains thereof that has a different activity of interest, resulting in a fusion protein.

In some cases, a heterologous polypeptide (a fusion partner) for use with an engineered CasX in the systems of the disclosure provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES) to escort the engineered CasX through the nuclear pore complex, a sequence to keep the fusion protein retained in the cytoplasm, a mitochondrial localization signal for targeting to the mitochondria, a chloroplast localization signal for targeting to a chloroplast, an ER retention signal, and the like). In some embodiments, a subject RNA-guided polypeptide or a conditionally active RNA-guided polypeptide and/or subject engineered CasX fusion protein does not include a NLS so that the protein is not targeted to the nucleus (which can be advantageous, e.g., when the target nucleic acid sequence is an RNA that is present in the cytosol). In some embodiments, a fusion partner can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

The disclosure contemplates assembly of multiple NLS in various configurations for linkage to the engineered CasX protein described herein. In some embodiments, a single NLS is linked at or near the N-terminus of the engineered CasX protein. In some embodiments, a single NLS is linked at or near the N-terminus and at or near the C-terminus of the engineered CasX protein. In some embodiments, the N-terminal NLS comprises one or more a c-MYC NLS. In some embodiments, the C-terminal NLS comprises one or more c-MYC NLS. In some embodiments, 2, 3, 4 or more NLS are linked by linker peptides at or near the C-terminus and/or the N-terminus of the engineered CasX protein. The person of ordinary skill in the art will understand that an NLS at or near the N- or C-terminus of a protein can be within 1, 2, 3, 4, 5, 6, 7, 8, 9, 10, 11, 12, 13, 14, 15, 16, 17, 18, 19 or 20 amino acids of the N- or C-terminus. In some embodiments, the NLS linked to the N-terminus of the engineered CasX protein are identical to the NLS linked to the C-terminus. In other embodiments, the NLS linked to the N-terminus of the engineered CasX protein are different to the NLS linked to the C-terminus. In some cases, non-limiting examples of NLSs suitable for use with an engineered CasX in the systems of the disclosure include sequences having at least about 80%, at least about 90%, or at least about 95% identity or are identical to sequences derived from: the NLS of the SV40 virus large T-antigen, having the amino acid sequence PKKKRKV (SEQ ID NO: 361); the NLS from nucleoplasmin (e.g., the nucleoplasmin bipartite NLS with the sequence KRPAATKKAGQAKKKK (SEQ ID NO: 362); the c-MYC NLS having the amino acid sequence PAAKRVKLD (SEQ ID NO: 363) or RQRRNELKRSP (SEQ ID NO: 364). In some embodiments, the NLS linked to the N-terminus of the engineered CasX protein is selected from the group consisting of the N-terminal sequences as set forth in Table 5. In some embodiments, the NLS linked to the C-terminus of the CasX protein is selected from the group consisting of the C-terminal sequences as set forth in Table 6. In some embodiments, NLSs suitable for use with an engineered CasX in the systems of the disclosure include sequences having at least about 80%, at least about 90%, or at least about 95% identity or are identical to one or more sequences of Table 5. The skilled artisan will appreciate that the NLS listed in Table 5 and Table 6 may be suitable for including in a fusion protein of the disclosure at or near either the N or C terminus of the fusion protein.

TABLE 5
N-terminal NLS Amino Acid Sequences

| | SEQ ID |
|---|---|
| NLS Amino Acid Sequence* | NO |
| PKKKRKVSR | 365 |
| PKKKRKVGGSPKKKRKVGGSPKKKRKVGGSPKKKRKVSR | 366 |
| PKKKRKVGGSPKKKRKVGGSPKKKRKVGGSPKKKRKVGGSPKKKRKVGGSPKKKRKVSR | 367 |
| PAAKRVKLDSR | 368 |
| PAAKRVKLDGGSPAAKRVKLDSR | 369 |
| PAAKRVKLDGGSPAAKRVKLDGGSPAAKRVKLDGGSPAAKRVKLDSR | 370 |
| PAAKRVKLDGGSPAAKRVKLDGGSPAAKRVKLDGGSPAAKRVKLDGGSPAAKRVKLDGGSPAAKRVKLD | 371 |
| SR | |
| KRPAATKKAGQAKKKKSR | 372 |
| KRPAATKKAGQAKKKKGGSKRPAATKKAGQAKKKKSR | 373 |
| PAAKRVKLDGGSPKKKRKVSR | 374 |
| PAAKKKKLDGGSPKKKRKVSR | 375 |
| PAAKKKKLDSR | 376 |
| PAAKKKKLDGGSPAAKKKKLDGGSPAAKKKKLDSR | 377 |
| PAAKKKKLDGGSPAAKKKKLDGGSPAAKKKKLDGGSPAAKKKKLDSR | 378 |
| PAKRARRGYKCSR | 379 |
| PAKRARRGYKCGSPAKRARRGYKCSR | 380 |
| PRRKREESR | 381 |
| PYRGRKESR | 382 |
| PLRKRPRRSR | 383 |
| PLRKRPRRGSPLRKRPRRSR | 384 |
| PAAKRVKLDGGKRTADGSEFESPKKKRKVGGS | 385 |
| PAAKRVKLDGGKRTADGSEFESPKKKRKVPPPPG | 386 |
| PAAKRVKLDGGKRTADGSEFESPKKKRKVGIHGVPAAPG | 387 |
| PAAKRVKLDGGKRTADGSEFESPKKKRKVGGGSGGGSPG | 388 |
| PAAKRVKLDGGKRTADGSEFESPKKKRKVPGGGSGGGSPG | 389 |
| PAAKRVKLDGGKRTADGSEFESPKKKRKVAEAAAKEAAAKEAAAKAPG | 390 |
| PAAKRVKLDGGSPKKKRKVGGS | 391 |
| PAAKRVKLDPPPPKKKRKVPG | 392 |
| PAAKRVKLDPG | 393 |
| PAAKRVKLDGGGSGGGSGGGS | 394 |
| PAAKRVKLDPPP | 395 |
| PAAKRVKLDGGGSGGGSGGGSPPP | 396 |
| PKKKRKVPPP | 397 |
| PKKKRKVGGS | 398 |

*Residues in bold are NLS residues, while unbolded residues are linkers.

TABLE 6
C-terminal NLS Amino Acid Sequences

| | SEQ ID |
|---|---|
| NLS Amino Acid Sequence | NO |
| GSPKKKRKVGGSPKKKRKVGGSPKKKRKVGGSPKKKRKV | 399 |
| GSPKKKRKVGGSPKKKRKVGGSPKKKRKVGGSPKKKRKVGGSPKKKRKVGGSPKKKRKV | 400 |
| GSPAAKRVKLDGGSPAAKRVKLD | 401 |
| GSPAAKRVKLDGGSPAAKRVKLDGGSPAAKRVKLDGGSPAAKRVKLD | 402 |
| GSKRPAATKKAGQAKKKK | 403 |
| KRPAATKKAGQAKKKKGGSKRPAATKKAGQAKKKK | 404 |
| GSKLGPRKATGRWGS | 405 |
| GSKRKGSPERGERKRHWGS | 406 |
| GSPKKKRKVGSGSKRPAATKKAGQAKKKKLE | 407 |
| GPKRTADSQHSTPPKTKRKVEFEPKKKRKV | 408 |
| GGGSGGGSKRTADSQHSTPPKTKRKVEFEPKKKRKV | 409 |
| AEAAAKEAAAKEAAAKAKRTADSQHSTPPKTKRKVEFEPKKKRKV | 410 |
| GPPKKKRKVGGSKRTADSQHSTPPKTKRKVEFEPKKKRKV | 411 |
| GPAEAAAKEAAAKEAAAKAPAAKRVKLD | 412 |
| GPGGGSGGGSGGGSPAAKRVKLD | 413 |
| GPPAAKRVKLD | 414 |
| VGSKRPAATKKAGQAKKKK | 415 |
| TGGGPGGGAAAGSGSPKKKRKVGSGSKRPAATKKAGQAKKKKLE | 416 |
| TGGGPGGGAAAGSGSPKKKRKVGSGS | 417 |
| PPPPKKKRKVPPP | 418 |
| GGSPKKKRKVPPP | 419 |
| PPPPKKKRKV | 420 |
| GGSPKKKRKV | 421 |
| GGSPKKKRKVGGSGGSGGS | 422 |
| GGSPKKKRKVGGSPKKKRKV | 423 |
| GGSGGSGGSPKKKRKVGGSPKKKRKV | 424 |
| VGGGSGGGSGGGSPAAKRVKLD | 425 |
| VPPPPAAKRVKLD | 426 |
| VPPPGGGSGGGSGGGSPAAKRVKLD | 427 |
| VGSPAAKRVKLD | 428 |

In some embodiments, the one or more NLSs are linked to the CasX protein or to adjacent NLS with a linker peptide wherein the linker peptide is selected from the group consisting of SR, GS, VGS, GGS, (G)n (SEQ ID NO: 429), (GS)n (SEQ ID NO: 430), (GSGGS)n (SEQ ID NO: 431), (GGSGGS)n (SEQ ID NO: 432), (GGGS)n (SEQ ID NO: 433), GGSG (SEQ ID NO: 434), GGSGG (SEQ ID NO: 435), GSGSG (SEQ ID NO: 436), GSGGG (SEQ ID NO: 437), GGGSG (SEQ ID NO: 438), GSSSG (SEQ ID NO: 439), GPGP (SEQ ID NO: 440), GGP, PPP, PPAPPA (SEQ ID NO: 441), PPPG (SEQ ID NO: 442), PPPGPPP (SEQ ID NO: 443), PPP(GGGS)n (SEQ ID NO: 444), (GGGS)nPPP (SEQ ID NO: 445), AEAAAKEAAAKEAAAKA (SEQ ID NO: 446), and TPPKTKRKVEFE (SEQ ID NO: 447), GGSGGGS (SEQ ID NO: 448), GSGSGGG (SEQ ID NO: 449), and SSGNSNANSRGPSFSSGLVPLSLRGSH (SEQ ID NO: 450), where n is 1 to 5.

In general, NLS (or multiple NLSs) are of sufficient strength to drive accumulation of an engineered CasX fusion protein in the nucleus of a eukaryotic cell. Detection of accumulation in the nucleus may be performed by any suitable technique. For example, a detectable marker may be fused to an engineered CasX fusion protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

f. mRNA Compositions Encoding Engineered CasX Proteins

In another aspect, the disclosure relates to modified messenger RNA (mRNA) compositions comprising sequences that encode engineered CasX proteins for use in the CasX:gRNA systems for use in certain delivery formulations; e.g., particles such as LNP. In some embodiments, the modified mRNA compositions have been designed to result in one or more of improved expression, reduced immunogenicity, increased stability, and enhanced manufacturability of the engineered CasX of the disclosure relative to CasX encoded by unmodified mRNA. The disclosure also provides methods utilized to design the compositions and formulations to deliver the compositions.

Modifications to an mRNA sequence can affect mRNA stability, protein translation and expression levels, and immunogenicity, and therefore have a significant impact on the efficacy of mRNA-based delivery. Optimization of coding sequences and untranslated regions (UTRs) may be particularly advantageous when delivering an mRNA encoding a protein of interest, as opposed to a DNA template that would be transcribed into an mRNA. DNA templates are long-lived, can replicate, and can produce many RNA transcripts over their lifetimes. For DNA templates, efficiency of transcription and pre-mRNA processing are major determinants of protein expression levels. In contrast, mRNAs generally have a much shorter half-life, on the order of hours, as they are vulnerable to degradation in the cytoplasm, and cannot produce more copies of themselves. As such, mRNA stability and translation efficiency are key determinants of protein expression levels for mRNA-based delivery, and the specific sequences of UTRs and coding sequences that dictate mRNA stability and translation efficiency can therefore be optimized to improve the efficacy of mRNA-based delivery.

The disclosure provides methods to generate modified mRNA sequences. In some embodiments, the methods comprising designing the mRNA sequences of the disclosure based using one or more parameters.

In some embodiments, the disclosure provides a modified mRNA sequence encoding CasX 515 (e.g., SEQ ID NO: 13741) for incorporation into a system of the disclosure, or a sequence having at least about 70%, at least about 80%, at least about 85%, at least about 90%, at least about 95%, at least about 96%, at least about 97%, at least about 98%, or having at least about 99% sequence identity thereto. In some embodiments, the disclosure provides a modified mRNA sequence encoding CasX 812 (e.g., SEQ ID NO: 13743) for incorporation into a system of the disclosure, or a sequence having at least about 70%, at least about 80%, at least about 85%, at least about 90%, at least about 95%, at least about 96%, at least about 97%, at least about 98%, or having at least about 99% sequence identity thereto. In some embodiments, the disclosure provides a modified mRNA sequence encoding CasX 676 (e.g., SEQ ID NO: 13743) for incorporation into a system of the disclosure, or a sequence having at least about 70%, at least about 80%, at least about 85%, at least about 90%, at least about 95%, at least about 96%, at least about 97%, at least about 98%, or having at least about 99% sequence identity thereto. In some embodiments, the disclosure provides a modified mRNA sequence encoding CasX 491 (e.g., SEQ ID NO: 13740) for incorporation into a system of the disclosure, or a sequence having at least about 70%, at least about 80%, at least about 85%, at least about 90%, at least about 95%, at least about 96%, at least about 97%, at least about 98%, or having at least about 99% sequence identity thereto. Representative but non-limiting mRNA sequences are presented in Table 7. In some embodiments the mRNA sequences of the disclosure may comprise one or more N1-methylpseudouridine substitutions for uridine, represented by my herein.

In some embodiments, the mRNA sequence encoding the engineered CasX further comprises a 5′ UTR and a 3′ UTR sequence. In some embodiments, the 3′ UTR sequence is derived from mouse hemoglobin alpha (mHBA). In some embodiments, the 5′ UTR comprises a sequence selected from the group consisting of SEQ ID NOS: 14049-14053. In some embodiments, the 3′ UTR comprises a sequence selected from the group consisting of SEQ ID NOS: 14046-14058.

TABLE 7
RNA sequences encoding CasX

| CasX | | SEQ | | SEQ |
|---|---|---|---|---|
| Desig- | | ID | | ID |
| nation | mRNA sequence | NO: | mRNA sequence* | NO: |
| 491 | CAGGAGAUCAAGCGGAUUAAUAAAA | 13740 | CAGGAGAmψCAAGCGGAmψmψAAmψAAAA | 13744 |
| | UUCGGAGAAGACUGGUGAAGGAUUC | | mψmψCGGAGAAGACmψGGmψGAAGGAmψm | |
| | UAACACAAAGAAGGCUGGCAAGACA | | ψCmψAACACAAAGAAGGCmψGGCAAGACA | |
| | GGCCCUAUGAAGACACUGCUGGUGA | | GGCCCmψAmψGAAGACACmψGCmψGGmψG | |
| | GAGUGAUGACACCCGACCUGAGAGA | | AGAGmψGAmψGACACCCGACCmψGAGAGA | |
| | AAGACUGGAAAACCUGAGAAAGAAG | | AAGACmψGGAAAACCmψGAGAAAGAAGCC | |
| | CCUGAGAAUAUCCCCCAGCCCAUCA | | mψGAGAAmψAmψCCCCCAGCCCAmψCAGC | |
| | GCAACACAAGCCGGGCCAACCUGAA | | AACACAAGCCGGGCCAACCmψGAAmψAAG | |
| | UAAGCUGCUGACCGACUACACCGAA | | CmψGCmψGACCGACmψACACCGAAAmψGA | |
| | AUGAAGAAGGCCAUCCUGCACGUGU | | AGAAGGCCAmψCCmψGCACGmψGmψAmψm | |
| | AUUGGGAAGAGUUCCAGAAAGACCC | | ψGGGAAGAGmψmψCCAGAAAGACCCAGmψ | |
| | AGUCGGCCUGAUGAGCAGAGUGGCU | | CGGCCmψGAmψGAGCAGAGmψGGCmψCAG | |
| | CAGCCUGCCAGCAAGAAGAUCGAUC | | CCmψGCCAGCAAGAAGAmψCGAmψCAGAA | |
| | AGAACAAGCUGAAGCCCGAAAUGGA | | CAAGCmψGAAGCCCGAAAmψGGACGAGAA | |
| | CGAGAAGGGGAACCUGACAACCGCC | | GGGGAACCmψGACAACCGCCGGCmψmψmψ | |
| | GGCUUUGCCUGUAGCCAGUGCGGCC | | GCCmwGmψAGCCAGmψGCGGCCAGCCCCm | |
| | AGCCCCUGUUUGUGUACAAACUGGA | | ψGmψmψmψGmψGmψACAAACmψGGAACAG | |
| | ACAGGUGAGCGAAAAGGGCAAGGCU | | GmwGAGCGAAAAGGGCAAGGCmψmψACAC | |
| | UACACGAAUUACUUCGGCAGAUGCA | | GAAmψmψACmψmψCGGCAGAmψGCAACGm | |
| | ACGUGGCCGAGCACGAGAAGCUGAU | | ψGGCCGAGCACGAGAAGCmψGAmψCCmψG | |
| | CCUGCUGGCCCAGCUGAAGCCUGAG | | CmψGGCCCAGCmψGAAGCCmψGAGAAGGA | |
| | AAGGAUAGCGAUGAGGCAGUGACAU | | mψAGCGAmψGAGGCAGmψGACAmψAmψmψ | |
| | AUUCCCUGGGCAAGUUCGGACAGCG | | CCCmψGGGCAAGmψmψCGGACAGCGGGCC | |
| | GGCCCUGGAUUUUUAUUCCAUUCAU | | CmψGGAmψmψmψmψmψAmψmψCCAmψmψC | |
| | GUGACCAAGGAAUCCACCCACCCCG | | AmψGmψGACCAAGGAAmψCCACCCACCCC | |
| | UCAAGCCUCUUGCCCAAAUUGCCGG | | GmψCAAGCCmψCmψmψGCCCAAAmψmψGC | |
| | CAACAGAUACGCCAGCGGCCCCGUG | | CGGCAACAGAmψACGCCAGCGGCCCCGmψ | |
| | GGCAAGGCCCUGAGCGACGCCUGUA | | GGGCAAGGCCCmψGAGCGACGCCmψGmψA | |
| | UGGGCACCAUCGCCAGCUUCCUGUC | | mψGGGCACCAmψCGCCAGCmψmψCCmψGm | |
| | UAAGUACCAGGACAUUAUCAUCGAG | | ψCmψAAGmψACCAGGACAmψmψAmψCAmψ | |
| | CACCAGAAGGUGGUGAAGGGCAACC | | CGAGCACCAGAAGGmψGGmψGAAGGGCAA | |
| | AGAAGAGACUGGAGAGCCUGCGCGA | | CCAGAAGAGACmψGGAGAGCCmψGCGCGA | |
| | GCUGGCCGGCAAGGAAAACCUGGAG | | GCmψGGCCGGCAAGGAAAACCmψGGAGmψ | |
| | UAUCCUAGCGUGACCCUGCCUCCUC | | AmψCCmψAGCGmψGACCCmψGCCmψCCmψ | |
| | AGCCUCAUACAAAGGAGGGCGUGGA | | CAGCCmψCAmψACAAAGGAGGGCGmψGGA | |
| | UGCCUACAACGAAGUGAUCGCCCGG | | mψGCCmψACAACGAAGmψGAmψCGCCCGG | |
| | GUGCGGAUGUGGGUGAACCUGAAUC | | GmψGCGGAmψGmψGGGmψGAACCmψGAAm | |
| | UGUGGCAGAAGCUGAAGCUGUCUAG | | ψCmψGmψGGCAGAAGCmψGAAGCmψGmψC | |
| | AGACGACGCCAAGCCCCUGCUGAGA | | mψAGAGACGACGCCAAGCCCCmψGCmψGA | |
| | CUGAAGGGCUUCCCCAGCUUCCCUC | | GACmψGAAGGGCmψmψCCCCAGCmψmψCC | |
| | UGGUGGAGAGACAGGCAAAUGAAGU | | CmψCmψGGmψGGAGAGACAGGCAAAmψGA | |
| | GGACUGGUGGGACAUGGUGUGUAAC | | AGmψGGACmψGGmψGGGACAmψGGmψGmψ | |
| | GUGAAGAAGCUGAUCAAUGAGAAGA | | GmψAACGmψGAAGAAGCmψGAmψCAAmψG | |
| | AGGAGGACGGCAAAGUGUUCUGGCA | | AGAAGAAGGAGGACGGCAAAGmψGmψmψC | |
| | GAAUCUGGCCGGCUACAAGCGUCAG | | mψGGCAGAAmψCmψGGCCGGCmψACAAGC | |
| | GAGGCCCUGCGGCCCUACCUGAGCA | | GmwCAGGAGGCCCmψGCGGCCCmψACCmψ | |
| | GCGAGGAAGACAGAAAGAAGGGCAA | | GAGCAGCGAGGAAGACAGAAAGAAGGGCA | |
| | GAAGUUCGCCCGGUAUCAGCUGGGG | | AGAAGmψmψCGCCCGGmψAmψCAGCmψGG | |
| | GACCUGCUGCUGCACCUCGAGAAGA | | GGGACCmψGCmψGCmψGCACCmψCGAGAA | |
| | AGCACGGCGAAGACUGGGGGAAGGU | | GAAGCACGGCGAAGACmlGGGGGAAGGmψ | |
| | GUACGAUGAGGCCUGGGAGCGGAUC | | GmψACGAmψGAGGCCmψGGGAGCGGAmψC | |
| | GAUAAGAAGGUGGAGGGCCUGAGCA | | GAmψAAGAAGGmψGGAGGGCCmψGAGCAA | |
| | AGCACAUCAAGCUGGAGGAGGAACG | | GCACAmψCAAGCmψGGAGGAGGAACGGAG | |
| | GAGAUCUGAGGACGCCCAGAGCAAG | | AmψCmψGAGGACGCCCAGAGCAAGGCCGC | |
| | GCCGCCCUGACCGACUGGCUGAGAG | | CCmψGACCGACmψGGCmψGAGAGCCAAGG | |
| | CCAAGGCCAGCUUCGUCAUCGAGGG | | CCAGCmψmψCGmψCAmψCGAGGGGCmψGA | |
| | GCUGAAGGAGGCCGACAAGGACGAG | | AGGAGGCCGACAAGGACGAGmψmψCmψGC | |
| | UUCUGCCGGUGCGAACUGAAGCUGC | | CGGmψGCGAACmψGAAGCmψGCAGAAGmψ | |
| | AGAAGUGGUACGGAGAUCUGAGAGG | | GGmψACGGAGAmψCmψGAGAGGCAAACCm | |
| | CAAACCUUUCGCCAUCGAGGCCGAG | | ψmψmψCGCCAmψCGAGGCCGAGAACAGCA | |
| | AACAGCAUCCUGGACAUCAGCGGCU | | mψCCmψGGACAmψCAGCGGCmψmψCAGCA | |
| | UCAGCAAGCAGUACAACUGCGCCUU | | AGCAGmψACAACmψGCGCCmψmψmψAmψm | |
| | UAUUUGGCAGAAGGACGGAGUGAAG | | ψmψGGCAGAAGGACGGAGmψGAAGAAGCm | |
| | AAGCUGAACCUGUACCUGAUCAUCA | | ψGAACCmψGmψACCmψGAmψCAmψCAACm | |
| | ACUAUUUCAAGGGCGGCAAGCUGAG | | ψAmψmψmψCAAGGGCGGCAAGCmψGAGAm | |
| | AUUCAAGAAGAUCAAGCCUGAAGCC | | ψmψCAAGAAGAmψCAAGCCmψGAAGCCmψ | |
| | UUCGAGGCCAACAGAUUCUACACCG | | mψCGAGGCCAACAGAmψmψCmψACACCGm | |
| | UGAUUAACAAGAAAAGCGGAGAGAU | | ψGAmψmψAACAAGAAAAGCGGAGAGAmψC | |
| | CGUGCCAAUGGAAGUGAACUUCAAC | | GmψGCCAAmψGGAAGmψGAACmψmψCAAC | |
| | UUCGACGACCCUAACCUGAUCAUCC | | mψmψCGACGACCCmψAACCmψGAmψCAmψ | |
| | UGCCCCUGGCAUUUGGCAAGCGGCA | | CCmψGCCCCmψGGCAmψmψmψGGCAAGCG | |
| | GGGCAGAGAGUUCAUCUGGAACGAC | | GCAGGGCAGAGAGmψmψCAmψCmψGGAAC | |
| | CUGCUGUCUCUGGAGACCGGCAGCC | | GACCmψGCmψGmψCmψCmψGGAGACCGGC | |
| | UGAAGCUGGCCAACGGCAGAGUGAU | | AGCCmψGAAGCmψGGCCAACGGCAGAGmψ | |
| | CGAGAAGACACUGUACAACAGACGA | | GAmψCGAGAAGACACmψGmψACAACAGAC | |
| | ACCAGACAAGACGAGCCCGCCCUGU | | GAACCAGACAAGACGAGCCCGCCCmψGmψ | |
| | UUGUGGCCCUGACCUUCGAGAGAAG | | mψmψGmψGGCCCmψGACCmψmψCGAGAGA | |
| | AGAGGUGCUGGACAGCAGCAAUAUC | | AGAGAGGmψGCmψGGACAGCAGCAAmψAm | |
| | AAGCCUAUGAACCUGAUCGGCGUGG | | ψCAAGCCmψAmψGAACCmψGAmψCGGCGm | |
| | ACCGGGGCGAGAACAUCCCUGCCGU | | ψGGACCGGGGCGAGAACAmψCCCmψGCCG | |
| | GAUCGCCCUUACCGACCCCGAGGGA | | mψGAmψCGCCCmψmψACCGACCCCGAGGG | |
| | UGCCCUCUGAGCCGGUUUAAAGACA | | AmψGCCCmψCmψGAGCCGGmψmψmψAAAG | |
| | GCCUGGGCAACCCUACCCACAUCCU | | ACAGCCmψGGGCAACCCmψACCCACAmψC | |
| | GAGAAUUGGCGAGUCCUACAAGGAG | | CmψGAGAAmψmψGGCGAGmψCCmψACAAG | |
| | AAGCAGAGAACCAUCCAGGCCAAGA | | GAGAAGCAGAGAACCAmψCCAGGCCAAGA | |
| | AGGAGGUGGAGCAGCGGCGGGCUGG | | AGGAGGmψGGAGCAGCGGCGGGCmψGGCG | |
| | CGGCUACUCCCGGAAGUACGCCAGC | | GCmψACmψCCCGGAAGmψACGCCAGCAAG | |
| | AAGGCCAAGAACCUGGCCGACGACA | | GCCAAGAACCmψGGCCGACGACAmψGGmψ | |
| | UGGUUAGAAAUACCGCCAGAGACCU | | mψAGAAAmψACCGCCAGAGACCmψCCmψG | |
| | CCUGUACUACGCUGUGACCCAGGAC | | mψACmψACGCmψGmψGACCCAGGACGCCA | |
| | GCCAUGCUGAUCUUCGAGAACCUGA | | mψGCmψGAmψCmψmψCGAGAACCmψGAGC | |
| | GCAGAGGCUUCGGCAGACAGGGCAA | | AGAGGCmψmψCGGCAGACAGGGCAAGAGA | |
| | GAGAACCUUCAUGGCCGAGAGACAG | | ACCmψmψCAmψGGCCGAGAGACAGmψACA | |
| | UACACCCGGAUGGAGGACUGGCUGA | | CCCGGAmψGGAGGACmψGGCmψGACCGCC | |
| | CCGCCAAGCUGGCCUACGAGGGCCU | | AAGCmψGGCCmψACGAGGGCCmψGmψCmψ | |
| | GUCUAAGACCUACCUGUCCAAGACC | | AAGACCmψACCmψGmψCCAAGACCmψmψG | |
| | UUGGCACAGUACACCAGCAAGACAU | | GCACAGmψACACCAGCAAGACAmψGCmψC | |
| | GCUCUAACUGCGGCUUCACAAUCAC | | mψAACmψGCGGCmψmψCACAAmψCACGAG | |
| | GAGCGCCGACUACGACCGGGUGCUG | | CGCCGACmψACGACCGGGmψGCmψGGAGA | |
| | GAGAAACUGAAGAAGACCGCCACAG | | AACmψGAAGAAGACCGCCACAGGCmψGGA | |
| | GCUGGAUGACCACCAUUAACGGCAA | | mψGACCACCAmψmψAACGGCAAGGAGCmψ | |
| | GGAGCUGAAGGUGGAGGGCCAGAUC | | GAAGGmψGGAGGGCCAGAmψCACCmψACm | |
| | ACCUACUACAACAGGUACAAACGGC | | ψACAACAGGmwACAAACGGCAGAACGmψG | |
| | AGAACGUGGUGAAGGACCUGAGCGU | | GmψGAAGGACCmψGAGCGmψGGAACmψGG | |
| | GGAACUGGAUAGACUGAGCGAGGAA | | AmψAGACmψGAGCGAGGAAAGCGmψAAAC | |
| | AGCGUAAACAAUGACAUCAGCAGCU | | AAmψGACAmψCAGCAGCmψGGACCAAGGG | |
| | GGACCAAGGGCCGGAGCGGCGAGGC | | CCGGAGCGGCGAGGCCCmψGAGCCmψGCm | |
| | CCUGAGCCUGCUGAAGAAGAGAUUC | | ψGAAGAAGAGAmψmψCmψCCCACAGACCA | |
| | UCCCACAGACCAGUGCAGGAGAAGU | | GmψGCAGGAGAAGmψmψCGmψGmψGmψCm | |
| | UCGUGUGUCUGAACUGCGGCUUCGA | | ψGAACmψGCGGCmψmψCGAGACCCACGCC | |
| | GACCCACGCCGACGAGCAAGCCGCC | | GACGAGCAAGCCGCCCmψGAACAmψCGCC | |
| | CUGAACAUCGCCCGGUCUUGGCUUU | | CGGmψCmψmψGGCmψmψmψmψCCmψGCGG | |
| | UCCUGCGGAGCCAGGAGUACAAGAA | | AGCCAGGAGmψACAAGAAGmψACCAGACA | |
| | GUACCAGACAAACAAGACCACAGGC | | AACAAGACCACAGGCAACACAGACAAGAG | |
| | AACACAGACAAGAGAGCCUUCGUCG | | AGCCmψmψCGmψCGAGACCmψGGCAGAGC | |
| | AGACCUGGCAGAGCUUCUACAGAAA | | mψmψCmψACAGAAAGAAGCmψGAAGGAGG | |
| | GAAGCUGAAGGAGGUGUGGAAGCCU | | mwGmψGGAAGCCmψGCCGmψG | |
| | GCCGUG | | | |
| 515 | CAGGAGAUCAAGCGGAUUAAUAAAA | 13741 | CAGGAGAmψCAAGCGGAmψmψAAmψAAAA | 13745 |
| | UUCGGAGAAGACUGGUGAAGGAUUC | | mψmψCGGAGAAGACmψGGmψGAAGGAmψm | |
| | UAACACAAAGAAGGCUGGCAAGACA | | ψCmψAACACAAAGAAGGCmψGGCAAGACA | |
| | GGCCCUAUGAAGACACUGCUGGUGA | | GGCCCmψAmψGAAGACACmψGCmψGGmψG | |
| | GAGUGAUGACACCCGACCUGAGAGA | | AGAGmψGAmψGACACCCGACCmψGAGAGA | |
| | AAGACUGGAAAACCUGAGAAAGAAG | | AAGACmψGGAAAACCmψGAGAAAGAAGCC | |
| | CCUGAGAAUAUCCCCCAGCCCAUCA | | mψGAGAAmψAmψCCCCCAGCCCAmψCAGC | |
| | GCAACACAAGCCGGGCCAACCUGAA | | AACACAAGCCGGGCCAACCmψGAAmψAAG | |
| | UAAGCUGCUGACCGACUACACCGAA | | CmψGCmψGACCGACmψACACCGAAAmψGA | |
| | AUGAAGAAGGCCAUCCUGCACGUGU | | AGAAGGCCAmψCCmψGCACGmψGmψAmψm | |
| | AUUGGGAAGAGUUCCAGAAAGACCC | | ψGGGAAGAGmψmψCCAGAAAGACCCAGmψ | |
| | AGUCGGCCUGAUGAGCAGAGUGGCU | | CGGCCmψGAmψGAGCAGAGmψGGCmψCAG | |
| | CAGCCUGCCAGCAAGAAGAUCGAUC | | CCmψGCCAGCAAGAAGAmψCGAmψCAGAA | |
| | AGAACAAGCUGAAGCCCGAAAUGGA | | CAAGCmψGAAGCCCGAAAmψGGACGAGAA | |
| | CGAGAAGGGGAACCUGACAACCGCC | | GGGGAACCmψGACAACCGCCGGCmψmψmψ | |
| | GGCUUUGCCUGUAGCCAGUGCGGCC | | GCCmψGmψAGCCAGmψGCGGCCAGCCCCm | |
| | AGCCCCUGUUUGUGUACAAACUGGA | | ψGmψmψmψGmψGmψACAAACmψGGAACAG | |
| | ACAGGUGAGCGAAAAGGGCAAGGCU | | GmψGAGCGAAAAGGGCAAGGCmψmψACAC | |
| | UACACGAAUUACUUCGGCAGAUGCA | | GAAmψmψACmψmψCGGCAGAmψGCAACGm | |
| | ACGUGGCCGAGCACGAGAAGCUGAU | | ψGGCCGAGCACGAGAAGCmψGAmψCCmψG | |
| | CCUGCUGGCCCAGCUGAAGCCUGAG | | CmψGGCCCAGCmψGAAGCCmψGAGAAGGA | |
| | AAGGAUAGCGAUGAGGCAGUGACAU | | mψAGCGAmψGAGGCAGmψGACAmψAmψmψ | |
| | AUUCCCUGGGCAAGUUCGGACAGCG | | CCCmψGGGCAAGmψmψCGGACAGCGGGCC | |
| | GGCCCUGGAUUUUUAUUCCAUUCAU | | CmψGGAmψmψmψmψmψAmψmψCCAmψmψC | |
| | GUGACCAAGGAAUCCACCCACCCCG | | AmψGmψGACCAAGGAAmψCCACCCACCCC | |
| | UCAAGCCUCUUGCCCAAAUUGCCGG | | GmψCAAGCCmψCmψmψGCCCAAAmψmψGC | |
| | CAACAGAUACGCCAGCGGCCCCGUG | | CGGCAACAGAmψACGCCAGCGGCCCCGmψ | |
| | GGCAAGGCCCUGAGCGACGCCUGUA | | GGGCAAGGCCCmψGAGCGACGCCmψGmψA | |
| | UGGGCACCAUCGCCAGCUUCCUGUC | | mψGGGCACCAmψCGCCAGCmψmψCCmψGm | |
| | UAAGUACCAGGACAUUAUCAUCGAG | | ψCmψAAGmψACCAGGACAmψmψAmψCAmψ | |
| | CACCAGAAGGUGGUGAAGGGCAACC | | CGAGCACCAGAAGGmψGGmψGAAGGGCAA | |
| | AGAAGAGACUGGAGAGCCUGCGCGA | | CCAGAAGAGACmψGGAGAGCCmψGCGCGA | |
| | GCUGGCCGGCAAGGAAAACCUGGAG | | GCmψGGCCGGCAAGGAAAACCmψGGAGmψ | |
| | UAUCCUAGCGUGACCCUGCCUCCUC | | AmψCCmψAGCGmψGACCCmψGCCmψCCmψ | |
| | AGCCUCAUACAAAGGAGGGCGUGGA | | CAGCCmψCAmψACAAAGGAGGGCGmψGGA | |
| | UGCCUACAACGAAGUGAUCGCCCGG | | mψGCCmψACAACGAAGmψGAmψCGCCCGG | |
| | GUGCGGAUGUGGGUGAACCUGAAUC | | GmψGCGGAmψGmψGGGmψGAACCmψGAAm | |
| | UGUGGCAGAAGCUGAAGCUGUCUAG | | ψCmψGmψGGCAGAAGCmψGAAGCmψGmψC | |
| | AGACGACGCCAAGCCCCUGCUGAGA | | mψAGAGACGACGCCAAGCCCCmψGCmψGA | |
| | CUGAAGGGCUUCCCCAGCUUCCCUC | | GACmψGAAGGGCmψmψCCCCAGCmψmψCC | |
| | UGGUGGAGAGACAGGCAAAUGAAGU | | CmψCmψGGmψGGAGAGACAGGCAAAmψGA | |
| | GGACUGGUGGGACAUGGUGUGUAAC | | AGmψGGACmψGGmψGGGACAmψGGmψGmψ | |
| | GUGAAGAAGCUGAUCAAUGAGAAGA | | GmψAACGmψGAAGAAGCmψGAmψCAAmψG | |
| | AGGAGGACGGCAAAGUGUUCUGGCA | | AGAAGAAGGAGGACGGCAAAGmψGmψmψC | |
| | GAAUCUGGCCGGCUACAAGCGUCAG | | mψGGCAGAAmψCmψGGCCGGCmψACAAGC | |
| | GAGGCCCUGCGGCCCUACCUGAGCA | | GmwCAGGAGGCCCmψGCGGCCCmψACCmψ | |
| | GCGAGGAAGACAGAAAGAAGGGCAA | | GAGCAGCGAGGAAGACAGAAAGAAGGGCA | |
| | GAAGUUCGCCCGGUAUCAGCUGGGG | | AGAAGmψmψCGCCCGGmψAmψCAGCmψGG | |
| | GACCUGCUGCUGCACCUCGAGAAGA | | GGGACCmψGCmψGCmψGCACCmψCGAGAA | |
| | AGCACGGCGAAGACUGGGGGAAGGU | | GAAGCACGGCGAAGACmψGGGGGAAGGmψ | |
| | GUACGAUGAGGCCUGGGAGCGGAUC | | GmψACGAmψGAGGCCmψGGGAGCGGAmψC | |
| | GAUAAGAAGGUGGAGGGCCUGAGCA | | GAmψAAGAAGGmψGGAGGGCCmψGAGCAA | |
| | AGCACAUCAAGCUGGAGGAGGAACG | | GCACAmψCAAGCmψGGAGGAGGAACGGAG | |
| | GAGAUCUGAGGACGCCCAGAGCAAG | | AmψCmψGAGGACGCCCAGAGCAAGGCCGC | |
| | GCCGCCCUGACCGACUGGCUGAGAG | | CCmψGACCGACmψGGCmψGAGAGCCAAGG | |
| | CCAAGGCCAGCUUCGUCAUCGAGGG | | CCAGCmψmψCGmψCAmψCGAGGGGCmψGA | |
| | GCUGAAGGAGGCCGACAAGGACGAG | | AGGAGGCCGACAAGGACGAGmψmψCmψGC | |
| | UUCUGCCGGUGCGAACUGAAGCUGC | | CGGmwGCGAACmψGAAGCmψGCAGAAGmψ | |
| | AGAAGUGGUACGGAGAUCUGAGAGG | | GGmψACGGAGAmψCmψGAGAGGCAAACCm | |
| | CAAACCUUUCGCCAUCGAGGCCGAG | | ψmψmwCGCCAmψCGAGGCCGAGAACAGCA | |
| | AACAGCAUCCUGGACAUCAGCGGCU | | mψCCmψGGACAmψCAGCGGCmψmψCAGCA | |
| | UCAGCAAGCAGUACAACUGCGCCUU | | AGCAGmψACAACmψGCGCCmψmψmψAmψm | |
| | UAUUUGGCAGAAGGACGGAGUGAAG | | ψmψGGCAGAAGGACGGAGmψGAAGAAGCm | |
| | AAGCUGAACCUGUACCUGAUCAUCA | | ψGAACCmψGmψACCmψGAmψCAmψCAACm | |
| | ACUAUUUCAAGGGCGGCAAGCUGAG | | ψAmψmψmψCAAGGGCGGCAAGCmψGAGAm | |
| | AUUCAAGAAGAUCAAGCCUGAAGCC | | ψmψCAAGAAGAmψCAAGCCmψGAAGCCmψ | |
| | UUCGAGGCCAACAGAUUCUACACCG | | mψCGAGGCCAACAGAmψmψCmψACACCGm | |
| | UGAUUAACAAGAAAAGCGGAGAGAU | | ψGAmψmψAACAAGAAAAGCGGAGAGAmψC | |
| | CGUGCCAAUGGAAGUGAACUUCAAC | | GmψGCCAAmψGGAAGmψGAACmψmψCAAC | |
| | UUCGACGACCCUAACCUGAUCAUCC | | mψmψCGACGACCCmψAACCmψGAmψCAmψ | |
| | UGCCCCUGGCAUUUGGCAAGCGGCA | | CCmψGCCCCmψGGCAmψmψmψGGCAAGCG | |
| | GGGCAGAGAGUUCAUCUGGAACGAC | | GCAGGGCAGAGAGmψmψCAmψCmψGGAAC | |
| | CUGCUGUCUCUGGAGACCGGCAGCC | | GACCmψGCmψGmψCmψCmψGGAGACCGGC | |
| | UGAAGCUGGCCAACGGCAGAGUGAU | | AGCCmψGAAGCmψGGCCAACGGCAGAGmψ | |
| | CGAGAAGACACUGUACAACAGACGA | | GAmψCGAGAAGACACmψGmψACAACAGAC | |
| | ACCAGACAAGACGAGCCCGCCCUGU | | GAACCAGACAAGACGAGCCCGCCCmψGmψ | |
| | UUGUGGCCCUGACCUUCGAGAGAAG | | mψmψGmψGGCCCmψGACCmψmψCGAGAGA | |
| | AGAGGUGCUGGACAGCAGCAAUAUC | | AGAGAGGmψGCmψGGACAGCAGCAAmψAm | |
| | AAGCCUAUGAACCUGAUCGGCGUGG | | ψCAAGCCmψAmψGAACCmψGAmψCGGCGm | |
| | ACCGGGGCGAGAACAUCCCUGCCGU | | ψGGACCGGGGCGAGAACAmψCCCmψGCCG | |
| | GAUCGCCCUUACCGACCCCGAGGGA | | mψGAmψCGCCCmψmψACCGACCCCGAGGG | |
| | UGCCCUCUGAGCCGGUUUAAAGACA | | AmψGCCCmψCmψGAGCCGGmψmψmψAAAG | |
| | GCCUGGGCAACCCUACCCACAUCCU | | ACAGCCmψGGGCAACCCmψACCCACAmψC | |
| | GAGAAUUGGCGAGUCCUACAAGGAG | | CmψGAGAAmψmψGGCGAGmψCCmψACAAG | |
| | AAGCAGAGAACCAUCCAGGCCAAGA | | GAGAAGCAGAGAACCAmψCCAGGCCAAGA | |
| | AGGAGGUGGAGCAGCGGCGGGCUGG | | AGGAGGmψGGAGCAGCGGCGGGCmψGGCG | |
| | CGGCUACUCCCGGAAGUACGCCAGC | | GCmψACmψCCCGGAAGmψACGCCAGCAAG | |
| | AAGGCCAAGAACCUGGCCGACGACA | | GCCAAGAACCmψGGCCGACGACAmψGGmψ | |
| | UGGUUAGAAAUACCGCCAGAGACCU | | mψAGAAAmψACCGCCAGAGACCmψCCmψG | |
| | CCUGUACUACGCUGUGACCCAGGAC | | mψACmψACGCmψGmψGACCCAGGACGCCA | |
| | GCCAUGCUGAUCUUCGAGAACCUGA | | mψGCmψGAmψCmψmψCGAGAACCmψGAGC | |
| | GCAGAGGCUUCGGCAGACAGGGCAA | | AGAGGCmψmψCGGCAGACAGGGCAAGAGA | |
| | GAGAACCUUCAUGGCCGAGAGACAG | | ACCmψmψCAmψGGCCGAGAGACAGmψACA | |
| | UACACCCGGAUGGAGGACUGGCUGA | | CCCGGAmψGGAGGACmψGGCmψGACCGCC | |
| | CCGCCAAGCUGGCCUACGAGGGCCU | | AAGCmψGGCCmψACGAGGGCCmψGCCCmψ | |
| | GCCCUCUAAGACCUACCUGUCCAAG | | CmψAAGACCmψACCmψGmψCCAAGACCmψ | |
| | ACCUUGGCACAGUACACCAGCAAGA | | mψGGCACAGmψACACCAGCAAGACAmψGC | |
| | CAUGCUCUAACUGCGGCUUCACAAU | | mψCmψAACmψGCGGCmψmψCACAAmψCAC | |
| | CACGAGCGCCGACUACGACCGGGUG | | GAGCGCCGACmψACGACCGGGmψGCmψGG | |
| | CUGGAGAAACUGAAGAAGACCGCCA | | AGAAACmψGAAGAAGACCGCCACAGGCmψ | |
| | CAGGCUGGAUGACCACCAUUAACGG | | GGAmψGACCACCAmψmψAACGGCAAGGAG | |
| | CAAGGAGCUGAAGGUGGAGGGCCAG | | CmψGAAGGmψGGAGGGCCAGAmψCACCmψ | |
| | AUCACCUACUACAACAGGUACAAAC | | ACmψACAACAGGmψACAAACGGCAGAACG | |
| | GGCAGAACGUGGUGAAGGACCUGAG | | mψGGmwGAAGGACCmψGAGCGmψGGAACm | |
| | CGUGGAACUGGAUAGACUGAGCGAG | | ψGGAmψAGACmψGAGCGAGGAAAGCGmψA | |
| | GAAAGCGUAAACAAUGACAUCAGCA | | AACAAmψGACAmψCAGCAGCmψGGACCAA | |
| | GCUGGACCAAGGGCCGGAGCGGCGA | | GGGCCGGAGCGGCGAGGCCCmψGAGCCmψ | |
| | GGCCCUGAGCCUGCUGAAGAAGAGA | | GCmψGAAGAAGAGAmψmψCmψCCCACAGA | |
| | UUCUCCCACAGACCAGUGCAGGAGA | | CCAGmψGCAGGAGAAGmψmψCGmψGmψGm | |
| | AGUUCGUGUGUCUGAACUGCGGCUU | | ψCmψGAACmψGCGGCmψmψCGAGACCCAC | |
| | CGAGACCCACGCCGACGAGCAAGCC | | GCCGACGAGCAAGCCGCCCmψGAACAmψC | |
| | GCCCUGAACAUCGCCCGGUCUUGGC | | GCCCGGmψCmψmψGGCmψmψmψmψCCmψG | |
| | UUUUCCUGCGGAGCCAGGAGUACAA | | CGGAGCCAGGAGmψACAAGAAGmψACCAG | |
| | GAAGUACCAGACAAACAAGACCACA | | ACAAACAAGACCACAGGCAACACAGACAA | |
| | GGCAACACAGACAAGAGAGCCUUCG | | GAGAGCCmψmψCGmψCGAGACCmψGGCAG | |
| | UCGAGACCUGGCAGAGCUUCUACAG | | AGCmψmψCmψACAGAAAGAAGCmψGAAGG | |
| | AAAGAAGCUGAAGGAGGUGUGGAAG | | AGGmψGmψGGAAGCCmψGCCGmψG | |
| | CCUGCCGUG | | | |
| 676 | CAGGAGAUCAAGCGGAUUAAUAAAA | 13742 | CAGGAGAmΨCAAGCGGAmΨmΨAAmΨAAAA | 13746 |
| | UUCGGAGAAGACUGGUGAAGGAUUC | | mΨmΨCGGAGAAGACmΨGGmΨGAAGGAmΨm | |
| | UAACACAAAGAAGGCUGGCAAGACA | | ΨCmΨAACACAAAGAAGGCmΨGGCAAGACA | |
| | CGGGGCCCUAUGAAGACACUGCUGG | | CGGGGCCCmΨAmΨGAAGACACmΨGCmΨGG | |
| | UGAGAGUGAUGACACCCGACCUGAG | | mΨGAGAGmΨGAmΨGACACCCGACCmΨGAG | |
| | AGAAAGACUGGAAAACCUGAGAAAG | | AGAAAGACmΨGGAAAACCmΨGAGAAAGAA | |
| | AAGCCUGAGAAUAUCCCCCAGCCCA | | GCCmΨGAGAAmΨAmΨCCCCCAGCCCAmΨC | |
| | UCAGCAACACAAGCCGGGCCAACCU | | AGCAACACAAGCCGGGCCAACCmΨGAAmΨ | |
| | GAAUAAGCUGCUGACCGACUACACC | | AAGCmΨGCmΨGACCGACmΨACACCGAAAm | |
| | GAAAUGAAGAAGGCCAUCCUGCACG | | ΨGAAGAAGGCCAmΨCCmΨGCACGmΨGmΨA | |
| | UGUAUUGGGAAGAGUUCCAGAAAGA | | mΨmΨGGGAAGAGmΨmΨCCAGAAAGACCCA | |
| | CCCAGUCGGCCUGAUGAGCAGAGUG | | GmΨCGGCCmΨGAmΨGAGCAGAGmΨGGCmΨ | |
| | GCUCAGCCUGCCAGCAAGAAGAUCG | | CAGCCmΨGCCAGCAAGAAGAmΨCGAmΨCA | |
| | AUCAGAACAAGCUGAAGCCCGAAAU | | GAACAAGCmΨGAAGCCCGAAAmΨGGACGA | |
| | GGACGAGAAGGGGAACCUGACAACC | | GAAGGGGAACCmΨGACAACCGCCGGCmΨm | |
| | GCCGGCUUUGCCUGUAGCCAGUGCG | | ΨmΨGCCmΨGmΨAGCCAGmΨGCGGCCAGCC | |
| | GCCAGCCCCUGUUUGUGUACAAACU | | CCmΨGmΨmΨmΨGmΨGmΨACAAACmΨGGAA | |
| | GGAACAGGUGAGCGAAAAGGGCAAG | | CAGGmΨGAGCGAAAAGGGCAAGGCmΨmΨA | |
| | GCUUACACGAAUUACUUCGGCAGAU | | CACGAAmΨmΨACmΨmΨCGGCAGAmΨGCAA | |
| | GCAACGUGGCCGAGCACGAGAAGCU | | CGmΨGGCCGAGCACGAGAAGCmΨGAmΨCA | |
| | GAUCAAGCUGGCCCAGCUGAAGCCU | | AGCmΨGGCCCAGCmΨGAAGCCmΨGAGAAG | |
| | GAGAAGGAUAGCGAUGAGGCAGUGA | | GAmΨAGCGAmΨGAGGCAGmΨGACAmΨAmΨ | |
| | CAUAUUCCCUGGGCAAGUUCGGACA | | mΨCCCmΨGGGCAAGmΨmΨCGGACAGCGGG | |
| | GCGGGCCCUGGAUUUUUAUUCCAUU | | CCCmΨGGAmΨmΨmΨmΨmΨAmΨmΨCCAmΨm | |
| | CAUGUGACCAAGGAAUCCACCCACC | | ΨCAmΨGmΨGACCAAGGAAmΨCCACCCACC | |
| | CCGUCAAGCCUCUUGCCCAAAUUGC | | CCGmΨCAAGCCmΨCmΨmΨGCCCAAAmΨmΨ | |
| | CGGCAACAGAUACGCCUCCAGCCCC | | GCCGGCAACAGAmΨACGCCmΨCCAGCCCC | |
| | GUGGGCAAGGCCCUGAGCGACGCCU | | GmΨGGGCAAGGCCCmΨGAGCGACGCCmΨG | |
| | GUAUGGGCACCAUCGCCAGCUUCCU | | mΨAmΨGGGCACCAmΨCGCCAGCmΨmΨCCm | |
| | GUCUAAGUACCAGGACAUUAUCAUC | | ΨGmΨCmΨAAGmΨACCAGGACAmΨmΨAmΨC | |
| | GAGCACCAGAAGGUGGUGAAGGGCA | | AmΨCGAGCACCAGAAGGmΨGGmΨGAAGGG | |
| | ACCAGAAGAGACUGGAGAGCCUGCG | | CAACCAGAAGAGACmΨGGAGAGCCmΨGCG | |
| | CGAGCUGGCCGGCAAGGAAAACCUG | | CGAGCmΨGGCCGGCAAGGAAAACCmΨGGA | |
| | GAGUAUCCUAGCGUGACCCUGCCUC | | GmΨAmΨCCmΨAGCGmΨGACCCmΨGCCmΨC | |
| | CUCAGCCUCAUACAAAGGAGGGCGU | | CmΨCAGCCmΨCAmΨACAAAGGAGGGCGmΨ | |
| | GGAUGCCUACAACGAAGUGAUCGCC | | GGAmΨGCCmΨACAACGAAGmΨGAmΨCGCC | |
| | CGGGUGCGGAUGUGGGUGAACCUGA | | CGGGmΨGCGGAmΨGmΨGGGmΨGAACCmΨG | |
| | AUCUGUGGCAGAAGCUGAAGCUGUC | | AAmΨCmΨGmΨGGCAGAAGCmΨGAAGCmΨG | |
| | UAGAGACGACGCCAAGCCCCUGCUG | | mΨCmΨAGAGACGACGCCAAGCCCCmΨGCm | |
| | AGACUGAAGGGCUUCCCCAGCUUCC | | ΨGAGACmΨGAAGGGCmmΨCCCCAGCmΨm | |
| | CUCUGGUGGAGAGACAGGCAAAUGA | | ΨCCCmΨCmΨGGmΨGGAGAGACAGGCAAAm | |
| | AGUGGACUGGUGGGACAUGGUGUGU | | ΨGAAGmΨGGACmΨGGmΨGGGACAmΨGGmΨ | |
| | AACGUGAAGAAGCUGAUCAAUGAGA | | GmΨGmΨAACGmΨGAAGAAGCmΨGAmΨCAA | |
| | AGAAGGAGGACGGCAAAGUGUUCUG | | mΨGAGAAGAAGGAGGACGGCAAAGmΨGmΨ | |
| | GCAGAAUCUGGCCGGCUACAAGCGU | | mΨCmΨGGCAGAAmΨCmΨGGCCGGCmΨACA | |
| | CAGGAGGCCCUGCGGCCCUACCUGA | | AGCGmΨCAGGAGGCCCmΨGCGGCCCmΨAC | |
| | GCAGCGAGGAAGACAGAAAGAAGGG | | CmΨGAGCAGCGAGGAAGACAGAAAGAAGG | |
| | CAAGAAGUUCGCCCGGUAUCAGCUG | | GCAAGAAGmΨmΨCGCCCGGmΨAmΨCAGCm | |
| | GGGGACCUGCUGCUGCACCUCGAGA | | ΨGGGGGACCmΨGCmΨGCmΨGCACCmΨCGA | |
| | AGAAGCACGGCGAAGACUGGGGGAA | | GAAGAAGCACGGCGAAGACmΨGGGGGAAG | |
| | GGUGUACGAUGAGGCCUGGGAGCGG | | GmΨGmΨACGAmΨGAGGCCmΨGGGAGCGGA | |
| | AUCGAUAAGAAGGUGGAGGGCCUGA | | mΨCGAmΨAAGAAGGmΨGGAGGGCCmΨGAG | |
| | GCAAGCACAUCAAGCUGGAGGAGGA | | CAAGCACAmΨCAAGCmΨGGAGGAGGAACG | |
| | ACGGAGAUCUGAGGACGCCCAGAGC | | GAGAmΨCmΨGAGGACGCCCAGAGCAAGGC | |
| | AAGGCCGCCCUGACCGACUGGCUGA | | CGCCCmΨGACCGACmΨGGCmΨGAGAGCCA | |
| | GAGCCAAGGCCAGCUUCGUCAUCGA | | AGGCCAGCmΨmΨCGmΨCAmΨCGAGGGGCm | |
| | GGGGCUGAAGGAGGCCGACAAGGAC | | ΨGAAGGAGGCCGACAAGGACGAGmΨmΨCm | |
| | GAGUUCUGCCGGUGCGAACUGAAGC | | ΨGCCGGmΨGCGAACmΨGAAGCmΨGCAGAA | |
| | UGCAGAAGUGGUACGGAGAUCUGAG | | GmΨGGmΨACGGAGAmΨCmΨGAGAGGCAAA | |
| | AGGCAAACCUUUCGCCAUCGAGGCC | | CCmΨmΨmΨCGCCAmΨCGAGGCCGAGAACA | |
| | GAGAACAGCAUCCUGGACAUCAGCG | | GCAmΨCCmΨGGACAmΨCAGCGGCmΨmΨCA | |
| | GCUUCAGCAAGCAGUACAACUGCGC | | GCAAGCAGmΨACAACmΨGCGCCmΨmΨmΨA | |
| | CUUUAUUUGGCAGAAGGACGGAGUG | | mΨmΨmΨGGCAGAAGGACGGAGmΨGAAGAA | |
| | AAGAAGCUGAACCUGUACCUGAUCA | | GCmΨGAACCmΨGmΨACCmΨGAmΨCAmΨCA | |
| | UCAACUAUUUCAAGGGCGGCAAGCU | | ACmΨAmΨmΨmΨCAAGGGCGGCAAGCmΨGA | |
| | GAGAUUCAAGAAGAUCAAGCCUGAA | | GAmΨmΨCAAGAAGAmΨCAAGCCmΨGAAGC | |
| | GCCUUCGAGGCCAACAGAUUCUACA | | CmΨmΨCGAGGCCAACAGAmΨmΨCmΨACAC | |
| | CCGUGAUUAACAAGAAAAGCGGAGA | | CGmΨGAmΨmΨAACAAGAAAAGCGGAGAGA | |
| | GAUCGUGCCAAUGGAAGUGAACUUC | | mΨCGmΨGCCAAmΨGGAAGmΨGAACmΨmΨC | |
| | AACUUCGACGACCCUAACCUGAUCA | | AACmΨmΨCGACGACCCmΨAACCmΨGAmΨC | |
| | UCCUGCCCCUGGCAUUUGGCAAGCG | | AmΨCCmΨGCCCCmΨGGCAmΨmΨmΨGGCAA | |
| | GCAGGGCAGAGAGUUCAUCUGGAAC | | GCGGCAGGGCAGAGAGmΨmΨCAmΨCmΨGG | |
| | GACCUGCUGUCUCUGGAGACCGGCA | | AACGACCmΨGCmΨGmΨCmΨCmΨGGAGACC | |
| | GCCUGAAGCUGGCCAACGGCAGAGU | | GGCAGCCmΨGAAGCmΨGGCCAACGGCAGA | |
| | GAUCGAGAAGACACUGUACAACAGA | | GmΨGAmΨCGAGAAGACACmΨGmΨACAACA | |
| | CGAACCAGACAAGACGAGCCCGCCC | | GACGAACCAGACAAGACGAGCCCGCCCmΨ | |
| | UGUUUGUGGCCCUGACCUUCGAGAG | | GmΨmΨmΨGmΨGGCCCmΨGACCmΨmΨCGAG | |
| | AAGAGAGGUGCUGGACAGCAGCAAU | | AGAAGAGAGGmΨGCmΨGGACAGCAGCAAm | |
| | AUCAAGCCUAUGAACCUGAUCGGCG | | ΨAmΨCAAGCCmΨAmΨGAACCmΨGAmΨCGG | |
| | UGGACCGGGGCGAGAACAUCCCUGC | | CGmΨGGACCGGGGCGAGAACAmΨCCCmΨG | |
| | CGUGAUCGCCCUUACCGACCCCGAG | | CCGmΨGAmΨCGCCCmΨmΨACCGACCCCGA | |
| | GGAUGCCCUCUGAGCCGGUUUAAAG | | GGGAmΨGCCCmΨCmΨGAGCCGGmΨmΨmΨA | |
| | ACAGCCUGGGCAACCCUACCCACAU | | AAGACAGCCmΨGGGCAACCCmΨACCCACA | |
| | CCUGAGAAUUGGCGAGUCCUACAAG | | mΨCCmΨGAGAAmΨmΨGGCGAGmΨCCmΨAC | |
| | GAGAAGCAGAGAACCAUCCAGGCCA | | AAGGAGAAGCAGAGAACCAmΨCCAGGCCA | |
| | AGAAGGAGGUGGAGCAGCGGCGGGC | | AGAAGGAGGmΨGGAGCAGCGGCGGGCmΨG | |
| | UGGCGGCUACUCCCGGAAGUACGCC | | GCGGCmΨACmΨCCCGGAAGmΨACGCCAGC | |
| | AGCAAGGCCAAGAACCUGGCCGACG | | AAGGCCAAGAACCmΨGGCCGACGACAmΨG | |
| | ACAUGGUUAGAAAUACCGCCAGAGA | | GmΨmΨAGAAAmΨACCGCCAGAGACCmΨCC | |
| | CCUCCUGUACUACGCUGUGACCCAG | | mΨGmΨACmΨACGCmΨGmΨGACCCAGGACG | |
| | GACGCCAUGCUGAUCUUCGAGAACC | | CCAmΨGCmΨGAmΨCmΨmΨCGAGAACCmΨG | |
| | UGAGCAGAGGCUUCGGCAGACAGGG | | AGCAGAGGCmΨmΨCGGCAGACAGGGCAAG | |
| | CAAGAGAACCUUCAUGGCCGAGAGA | | AGAACCmΨmΨCAmΨGGCCGAGAGACAGmΨ | |
| | CAGUACACCCGGAUGGAGGACUGGC | | ACACCCGGAmΨGGAGGACmΨGGCmΨGACC | |
| | UGACCGCCAAGCUGGCCUACGAGGG | | GCCAAGCmΨGGCCmΨACGAGGGCCmΨGCC | |
| | CCUGCCCUCUAAGACCUACCUGUCC | | CmΨCmΨAAGACCmΨACCmΨGmΨCCAAGAC | |
| | AAGACCUUGGCACAGUACACCAGCA | | CmΨmΨGGCACAGmΨACACCAGCAAGACAm | |
| | AGACAUGCUCUAACUGCGGCUUCAC | | ΨGCmΨCmΨAACmΨGCGGCmΨmΨCACAAmΨ | |
| | AAUCACGAGCGCCGACUACGACCGG | | CACGAGCGCCGACmΨACGACCGGGmΨGCm | |
| | GUGCUGGAGAAACUGAAGAAGACCG | | ΨGGAGAAACmΨGAAGAAGACCGCCACAGG | |
| | CCACAGGCUGGAUGACCACCAUUAA | | CmΨGGAmΨGACCACCAmΨmΨAACGGCAAG | |
| | CGGCAAGGAGCUGAAGGUGGAGGGC | | GAGCmΨGAAGGmΨGGAGGGCCAGAmΨCAC | |
| | CAGAUCACCUACUACAACAGGUACA | | CmΨACmΨACAACAGGmΨACAAACGGCAGA | |
| | AACGGCAGAACGUGGUGAAGGACCU | | ACGmΨGGmΨGAAGGACCmΨGAGCGmΨGGA | |
| | GAGCGUGGAACUGGAUAGACUGAGC | | ACmΨGGAmΨAGACmΨGAGCGAGGAAAGCG | |
| | GAGGAAAGCGUAAACAAUGACAUCA | | mΨAAACAAmΨGACAmΨCAGCAGCmΨGGAC | |
| | GCAGCUGGACCAAGGGCCGGAGCGG | | CAAGGGCCGGAGCGGCGAGGCCCmΨGAGC | |
| | CGAGGCCCUGAGCCUGCUGAAGAAG | | CmΨGCmΨGAAGAAGAGAmΨmΨCmΨCCCAC | |
| | AGAUUCUCCCACAGACCAGUGCAGG | | AGACCAGmΨGCAGGAGAAGmΨmΨCGmΨGm | |
| | AGAAGUUCGUGUGUCUGAACUGCGG | | ΨGmΨCmΨGAACmΨGCGGCmΨmΨCGAGACC | |
| | CUUCGAGACCCACGCCGACGAGCAA | | CACGCCGACGAGCAAGCCGCCCmΨGAACA | |
| | GCCGCCCUGAACAUCGCCCGGUCUU | | mΨCGCCCGGmΨCmΨmΨGGCmΨmΨmΨmΨCC | |
| | GGCUUUUCCUGCGGAGCCAGGAGUA | | mΨGCGGAGCCAGGAGmΨACAAGAAGmΨAC | |
| | CAAGAAGUACCAGACAAACAAGACC | | CAGACAAACAAGACCACAGGCAACACAGA | |
| | ACAGGCAACACAGACAAGAGAGCCU | | CAAGAGAGCCmΨmΨCGmΨCGAGACCmΨGG | |
| | UCGUCGAGACCUGGCAGAGCUUCUA | | CAGAGCmΨmΨCmΨACAGAAAGAAGCmΨGA | |
| | CAGAAAGAAGCUGAAGGAGGUGUGG | | AGGAGGmGmΨGGAAGCCmΨGCCGmΨG | |
| | AAGCCUGCCGUG | | | |
| 812 | CAGGAGAUCAAGCGGAUUAAUAAAA | 13743 | CAGGAGAmψCAAGCGGAmψmψAAmψAAAA | 13747 |
| | UUCGGAGAAGACUGGUGAAGGAUUC | | mψmψCGGAGAAGACmψGGmψGAAGGAmψm | |
| | UAACACAAAGAAGGCUGGCAAGACA | | ψCmψAACACAAAGAAGGCmψGGCAAGACA | |
| | GGCCCUAUGAAGACACUGCUGGUGA | | GGCCCmψAmψGAAGACACmψGCmψGGmψG | |
| | GAGUGAUGACACCCGACCUGAGAGA | | AGAGmψGAmψGACACCCGACCmψGAGAGA | |
| | AAGACUGGAAAACCUGAGAAAGAAG | | AAGACmψGGAAAACCmψGAGAAAGAAGCC | |
| | CCUGAGAAUAUCCCCCAGCCCAUCA | | mψGAGAAmψAmψCCCCCAGCCCAmψCAGC | |
| | GCAACACAAGCCGGGCCAACCUGAA | | AACACAAGCCGGGCCAACCmψGAAmψAAG | |
| | UAAGCUGCUGACCGACUACACCGAA | | CmψGCmψGACCGACmψACACCGAAAmψGA | |
| | AUGAAGAAGGCCAUCCUGCACGUGU | | AGAAGGCCAmψCCmψGCACGmψGmψAmψm | |
| | AUUGGGAAGAGUUCCAGAAAGACCC | | ψGGGAAGAGmψmψCCAGAAAGACCCAGmψ | |
| | AGUCGGCCUGAUGAGCAGAGUGGCU | | CGGCCmψGAmψGAGCAGAGmψGGCmψCAG | |
| | CAGCCUGCCAGCAAGAAGAUCGAUC | | CCmψGCCAGCAAGAAGAmψCGAmψCAGAA | |
| | AGAACAAGCUGAAGCCCGAAAUGGA | | CAAGCmψGAAGCCCGAAAmψGGACGAGAA | |
| | CGAGAAGGGGAACCUGACAACCGCC | | GGGGAACCmψGACAACCGCCGGCmψmψmψ | |
| | GGCUUUGCCUGUAGCCAGUGCGGCC | | GCCmψGmψAGCCAGmψGCGGCCAGCCCCm | |
| | AGCCCCUGUUUGUGUACAAACUGGA | | ψGmψmψmψGmψGmψACAAACmψGGAACAG | |
| | ACAGGUGAGCGAAAAGGGCAAGGCU | | GmψGAGCGAAAAGGGCAAGGCmψmψACAC | |
| | UACACGAAUUACUUCGGCAGAUGCA | | GAAmψmψACmψmψCGGCAGAmψGCAACGm | |
| | ACGUGGCCGAGCACGAGAAGCUGAU | | ψGGCCGAGCACGAGAAGCmψGAmψCCmψG | |
| | CCUGCUGGCCCAGCUGAAGCCUGAG | | CmψGGCCCAGCmψGAAGCCmψGAGAAGGA | |
| | AAGGAUAGCGAUGAGGCAGUGACAU | | mψAGCGAmψGAGGCAGmψGACAmψAmψmψ | |
| | AUUCCCUGGGCAAGUUCGGACAGCG | | CCCmψGGGCAAGmψmψCGGACAGCGGGCC | |
| | GGCCCUGGAUUUUUAUUCCAUUCAU | | CmψGGAmψmψmψmψmψAmψmψCCAmψmψC | |
| | GUGACCAAGGAAUCCACCCACCCCG | | AmψGmψGACCAAGGAAmψCCACCCACCCC | |
| | UCAAGCCUCUUGCCCAAAUUGCCGG | | GmψCAAGCCmψCmψmψGCCCAAAmψmψGC | |
| | CAACAGAUACGCCAGCGGCCCCGUG | | CGGCAACAGAmψACGCCAGCGGCCCCGmψ | |
| | GGCAAGGCCCUGAGCGACGCCUGUA | | GGGCAAGGCCCmψGAGCGACGCCm/GmψA | |
| | UGGGCACCAUCGCCAGCUUCCUGUC | | mψGGGCACCAmψCGCCAGCmψmψCCmψGm | |
| | UAAGUACCAGGACAUUAUCAUCGAG | | ψCmψAAGmψACCAGGACAmψmψAmψCAmψ | |
| | CACCAGAAGGUGGUGAAGGGCAACC | | CGAGCACCAGAAGGmψGGmψGAAGGGCAA | |
| | AGAAGAGACUGGAGAGCCUGCGCGA | | CCAGAAGAGACmψGGAGAGCCmψGCGCGA | |
| | GCUGGCCGGCAAGGAAAACCUGGAG | | GCmψGGCCGGCAAGGAAAACCmψGGAGmψ | |
| | UAUCCUAGCGUGACCCUGCCUCCUC | | AmψCCmψAGCGmψGACCCmψGCCmψCCmψ | |
| | AGCCUCAUACAAAGGAGGGCGUGGA | | CAGCCmψCAmψACAAAGGAGGGCGmψGGA | |
| | UGCCUACAACGAAGUGAUCGCCCGG | | mψGCCmψACAACGAAGmψGAmψCGCCCGG | |
| | GUGCGGAUGUGGGUGAACCUGAAUC | | GmψGCGGAmψGmψGGGmψGAACCmψGAAm | |
| | UGUGGCAGAAGCUGAAGCUGUCUAG | | ψCmψGmψGGCAGAAGCmψGAAGCmψGmψC | |
| | AGACGACGCCAAGCCCCUGCUGAGA | | mψAGAGACGACGCCAAGCCCCmψGCmψGA | |
| | CUGAAGAAGUUCCCCAGCUUCCCUC | | GACmψGAAGAAGmψmψCCCCAGCmψmψCC | |
| | UGGUGGAGAGACAGGCAAAUGAAGU | | CmψCmψGGmψGGAGAGACAGGCAAAmψGA | |
| | GGACUGGUGGGACAUGGUGUGUAAC | | AGmψGGACmψGGmψGGGACAmψGGmψGmψ | |
| | GUGAAGAAGCUGAUCAAUGAGAAGA | | GmψAACGmψGAAGAAGCmψGAmψCAAmψG | |
| | AGGAGGACGGCAAAGUGUUCUGGCA | | AGAAGAAGGAGGACGGCAAAGmψGmψmψC | |
| | GAAUCUGGCCGGCUACAAGCGUCAG | | mψGGCAGAAmψCmψGGCCGGCmψACAAGC | |
| | GAGGCCCUGCGGCCCUACCUGAGCA | | GmψCAGGAGGCCCmψGCGGCCCmψACCmψ | |
| | GCGAGGAAGACAGAAAGAAGGGCAA | | GAGCAGCGAGGAAGACAGAAAGAAGGGCA | |
| | GAAGUUCGCCCGGUAUCAGCUGGGG | | AGAAGmψmψCGCCCGGmψAmψCAGCmψGG | |
| | GACCUGCUGCUGCACCUCGAGAAGA | | GGGACCmwGCmψGCmψGCACCmψCGAGAA | |
| | AGCACGGCGAAGACUGGGGGAAGGU | | GAAGCACGGCGAAGACmψGGGGGAAGGmψ | |
| | GUACGAUGAGGCCUGGGAGCGGAUC | | GmψACGAmψGAGGCCmψGGGAGCGGAmψC | |
| | GAUAAGAAGGUGGAGGGCCUGAGCA | | GAmψAAGAAGGmψGGAGGGCCmψGAGCAA | |
| | AGCACAUCAAGCUGGAGGAGGAACG | | GCACAmψCAAGCmψGGAGGAGGAACGGAG | |
| | GAGAUCUGAGGACGCCCAGAGCAAG | | AmψCmψGAGGACGCCCAGAGCAAGGCCGC | |
| | GCCGCCCUGACCGACUGGCUGAGAG | | CCmψGACCGACmψGGCmψGAGAGCCAAGG | |
| | CCAAGGCCAGCUUCGUCAUCGAGGG | | CCAGCmψmψCGmψCAmψCGAGGGGCmψGA | |
| | GCUGAAGGAGGCCGACAAGGACGAG | | AGGAGGCCGACAAGGACGAGmψmψCmψGC | |
| | UUCUGCCGGUGCGAACUGAAGCUGC | | CGGmψGCGAACmψGAAGCmψGCAGAAGmψ | |
| | AGAAGUGGUACGGAGAUCUGAGAGG | | GGmwACGGAGAmψCmψGAGAGGCAAACCm | |
| | CAAACCUUUCGCCAUCGAGGCCGAG | | ψmψmψCGCCAmψCGAGGCCGAGAACAGCA | |
| | AACAGCAUCCUGGACAUCAGCGGCU | | mψCCmψGGACAmψCAGCGGCmψmψCAGCA | |
| | UCAGCAAGCAGUACAACUGCGCCUU | | AGCAGmψACAACmψGCGCCmψmψmψAmψm | |
| | UAUUUGGCAGAAGGACGGAGUGAAG | | ψmψGGCAGAAGGACGGAGmψGAAGAAGCm | |
| | AAGCUGAACCUGUACCUGAUCAUCA | | ψGAACCmψGmψACCmψGAmψCAmψCAACm | |
| | ACUAUUUCAAGGGCGGCAAGCUGAG | | ψAmψmψmψCAAGGGCGGCAAGCmψGAGAm | |
| | AUUCAAGAAGAUCAAGCCUGAAGCC | | ψmψCAAGAAGAmψCAAGCCmψGAAGCCmψ | |
| | UUCGAGGCCAACAGAUUCUACACCG | | mψCGAGGCCAACAGAmψmψCmψACACCGm | |
| | UGAUUAACAAGAAAAGCGGAGAGAU | | ψGAmψmψAACAAGAAAAGCGGAGAGAmψC | |
| | CGUGCCAAUGGAAGUGAACUUCAAC | | GmψGCCAAmψGGAAGmψGAACmψmψCAAC | |
| | UUCGACGACCCUAACCUGAUCAUCC | | mψmψCGACGACCCmψAACCmψGAmψCAmψ | |
| | UGCCCCUGGCAUUUGGCAAGCGGCA | | CCmψGCCCCmψGGCAmψmψmψGGCAAGCG | |
| | GGGCAGAGAGUUCAUCUGGAACGAC | | GCAGGGCAGAGAGmψmψCAmψCmψGGAAC | |
| | CUGCUGUCUCUGGAGACCGGCAGCC | | GACCmψGCmψGmψCmψCmψGGAGACCGGC | |
| | UGAAGCUGGCCAACGGCAGAGUGAU | | AGCCmψGAAGCmψGGCCAACGGCAGAGmψ | |
| | CGAGAAGACACUGUACAACAGACGA | | GAmψCGAGAAGACACmψGmψACAACAGAC | |
| | ACCAGACAAGACGAGCCCGCCCUGU | | GAACCAGACAAGACGAGCCCGCCCmψGmψ | |
| | UUGUGGCCCUGACCUUCGAGAGAAG | | mψmψGmψGGCCCmψGACCmψmψCGAGAGA | |
| | AGAGGUGCUGGACAGCAGCAAUAUC | | AGAGAGGmψGCmψGGACAGCAGCAAmψAm | |
| | AAGCCUAUGAACCUGAUCGGCGUGG | | ψCAAGCCmψAmψGAACCmψGAmψCGGCGm | |
| | ACCGGGGCGAGAACAUCCCUGCCGU | | ψGGACCGGGGCGAGAACAmψCCCmψGCCG | |
| | GAUCGCCCUUACCGACCCCGAGGGA | | mψGAmψCGCCCmψmψACCGACCCCGAGGG | |
| | UGCCCUCUGAGCCGGUUUAAAGACA | | AmψGCCCmψCmψGAGCCGGmψmψmψAAAG | |
| | GCCUGGGCAACCCUACCCACAUCCU | | ACAGCCmψGGGCAACCCmψACCCACAmψC | |
| | GAGAAUUGGCGAGUCCUACAAGGAG | | CmψGAGAAmψmψGGCGAGmψCCmψACAAG | |
| | AAGCAGAGAACCAUCCAGGCCAAGA | | GAGAAGCAGAGAACCAmψCCAGGCCAAGA | |
| | AGGAGGUGGAGCAGCGGCGGGCUGG | | AGGAGGmψGGAGCAGCGGCGGGCmψGGCG | |
| | CGGCUACUCCCGGAAGUACGCCAGC | | GCmψACmψCCCGGAAGmψACGCCAGCAAG | |
| | AAGGCCAAGAACCUGGCCGACGACA | | GCCAAGAACCmψGGCCGACGACAmψGGmψ | |
| | UGGUUAGAAAUACCGCCAGAGACCU | | mψAGAAAmψACCGCCAGAGACCmψCCmψG | |
| | CCUGUACUACGCUGUGACCCAGGAC | | mψACmψACGCmψGmψGACCCAGGACGCCA | |
| | GCCAUGCUGAUCUUCGAGAACCUGA | | mψGCmψGAmψCmψmψCGAGAACCmψGAGC | |
| | GCAGAGGCUUCGGCAGACAGGGCAA | | AGAGGCmψmψCGGCAGACAGGGCAAGAGA | |
| | GAGAACCUUCAUGGCCGAGAGACAG | | ACCmψmψCAmψGGCCGAGAGACAGmψACA | |
| | UACACCCGGAUGGAGGACUGGCUGA | | CCCGGAmψGGAGGACmψGGCmψGACCGCC | |
| | CCGCCAAGCUGGCCUACGAGGGCCU | | AAAGCmψGGCCVACGAGGGCCmψGCCCmψ | |
| | GCCCUCUAAGACCUACCUGUCCAAG | | CmψAAGACCmψACCmψGmψCCAAGACCmψ | |
| | ACCUUGGCACAGUACACCAGCAAGA | | mψGGCACAGmψACACCAGCAAGACAmψGC | |
| | CAUGCUCUAACUGCGGCUUCACAAU | | mψCmψAACmψGCGGCmψmψCACAAmψCAC | |
| | CACGAGCGCCGACUACGACCGGGUG | | GAGCGCCGACmψACGACCGGGmψGCmψGG | |
| | CUGGAGAAACUGAAGAAGACCGCCA | | AGAAACmψGAAGAAGACCGCCACAGGCmψ | |
| | CAGGCUGGAUGACCACCAUUAACGG | | GGAmψGACCACCAmψmψAACGGCAAGGAG | |
| | CAAGGAGCUGAAGGUGGAGGGCCAG | | CmψGAAGGmψGGAGGGCCAGAmψCACCmψ | |
| | AUCACCUACUACAACAGGUACAAAC | | ACmψACAACAGGmψACAAACGGCAGAACG | |
| | GGCAGAACGUGGUGAAGGACCUGAG | | mψGGmwGAAGGACCmψGAGCGmψGGAACm | |
| | CGUGGAACUGGAUAGACUGAGCGAG | | ψGGAmψAGACmψGAGCGAGGAAAGCGmψA | |
| | GAAAGCGUAAACAAUGACAUCAGCA | | AACAAmψGACAmψCAGCAGCmψGGACCAA | |
| | GCUGGACCAAGGGCCGGAGCGGCGA | | GGGCCGGAGCGGCGAGGCCCmψGAGCCmψ | |
| | GGCCCUGAGCCUGCUGAAGAAGAGA | | GCmψGAAGAAGAGAmψmψCmψCCCACAGA | |
| | UUCUCCCACAGACCAGUGCAGGAGA | | CCAGmψGCAGGAGAAGmψmψCGmψGmψGm | |
| | AGUUCGUGUGUCUGAACUGCGGCUU | | ψCmψGAACmψGCGGCmψmψCGAGACCCAC | |
| | CGAGACCCACGCCGACGAGCAAGCC | | GCCGACGAGCAAGCCGCCCmψGAACAmψC | |
| | GCCCUGAACAUCGCCCGGUCUUGGC | | GCCCGGmψCmψmψGGCmψmψmψmψCCmψG | |
| | UUUUCCUGCGGAGCCAGGAGUACAA | | CGGAGCCAGGAGmψACAAGAAGmψACCAG | |
| | GAAGUACCAGACAAACAAGACCACA | | ACAAACAAGACCACAGGCAACACAGACAA | |
| | GGCAACACAGACAAGAGAGCCUUCG | | GAGAGCCmψmψCGmψCGAGACCmψGGCAG | |
| | UCGAGACCUGGCAGAGCUUCUACAG | | AGCmψmψCmψACAGAAAGAAGCmψGAAGG | |
| | AAAGAAGCUGAAGGAGGUGUGGAAG | | AGGmψGmψGGAAGCCmψGCCGmψG | |
| | CCUGCCGUG | | | |

*‘mψ’ indicates N1-methylpseudouridine substitutions for uridine

Various naturally-occurring or modified nucleosides may be used to produce mRNAs according to the present disclosure. In some embodiments, an mRNA consists essentially of, or comprises, natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the mRNA comprises one or more nonstandard nucleotide residues. The nonstandard nucleotide residues may include, e.g., 5-methyl-cytidine (“5 mC”), pseudouridine (“U”), and/or 2-thio-uridine (“2sU”). In particular embodiments, one or more of the uridine residues of the mRNA of the disclosure are replaced with N1-methyl-pseudouridine. See, e.g., U.S. Pat. No. 8,278,036 or WO2011012316, incorporated by reference herein, for a discussion of such residues and their incorporation into mRNA. In some embodiments, at least about 70%, at least about 80%, at least about 90%, at least about 95%, at least about 99%, or 100% of the uridine nucleosides of the mRNA sequence are replaced with N1-methylpseudouridine. In some embodiments, the mRNA sequence comprising N1-methylpseudouridines and encoding CasX 515 comprises the sequence of SEQ ID NO: 13741. In some embodiments, the mRNA sequence comprising N1-methylpseudouridines and encoding CasX 491 comprises the sequence of SEQ ID NO: 13740. In some embodiments, the mRNA sequence comprising N1-methylpseudouridines and encoding CasX 676 comprises the sequence of SEQ ID NO: 13742. In some embodiments, the mRNA sequence comprising N1-methylpseudouridines and encoding CasX 812 comprises the sequence of SEQ ID NO: 13743.

### IV. Guide Nucleic Acids of the Systems for Genetic Editing of Target Nucleic Acid

In another aspect, the disclosure relates to specifically-designed guide ribonucleic acids (gRNA) comprising a scaffold and a linked targeting sequence complementary to (and therefore able to hybridize with) a target nucleic acid sequence of a PCSK9 gene. gRNAs of the disclosure have utility in genome editing of the PCSK9 target nucleic acid in a eukaryotic cell. As used herein, the term “gRNA” covers naturally-occurring molecules and gRNA variants, including chimeric gRNA variants comprising domains from different gRNAs (referred to herein as chimeric gRNAs).

The disclosure provides systems comprising an mRNA encoding an engineered CasX protein and a gRNA as a CasX:gRNA system designed, upon expression of the engineered CasX protein in a transfected cell, to form a ribonucleoprotein (RNP) complex of the CasX protein with the gRNA. The RNP targets and edits specific locations in the target nucleic acid sequence of the cell. The gRNA provides target specificity to the RNP by including a targeting sequence (or “spacer”) having a nucleotide sequence that is complementary to a sequence of the target nucleic acid sequence, while the CasX protein of the system provides the site-specific activity such as cleavage or nicking of the target sequence. The CasX protein is guided to a target site (e.g., stabilized at a target site) within a target nucleic acid sequence by virtue of its association with the gRNA.

Embodiments of gRNAs and formulations of mRNAs and gRNAs for use in the editing of PCSK9 target nucleic acids are described herein, below.

a. Reference gRNA and gRNA Variants

As used herein, a “reference gRNA” refers to a CRISPR guide ribonucleic acid comprising a wild-type sequence of a naturally-occurring gRNA. In some embodiments, a gRNA scaffold of the disclosure may be subjected to one or more mutagenesis methods, such as the mutagenesis methods described in WO2022120095A1 and WO2020247882A1, incorporated by reference herein, which may include Deep Mutational Evolution (DME), deep mutational scanning (DMS), error prone PCR, cassette mutagenesis, random mutagenesis, staggered extension PCR, gene shuffling, domain swapping, or chemical modification to generate one or more gRNA variants with enhanced or varied properties relative to the gRNA scaffold that was modified. The activity of the gRNA scaffold from which a gRNA variant was derived may be used as a benchmark against which the activity of the gRNA variant is compared, thereby measuring improvements in function or other characteristics of the gRNA scaffold.

Table 8 provides the sequences of reference gRNA tracr and scaffold sequences. In some embodiments, the disclosure provides gRNA sequences wherein the gRNA has a scaffold comprising a sequence having one or more nucleotide modifications relative to a reference gRNA sequence of any one of SEQ ID NOS: 451-463 of Table 8.

TABLE 8
Reference gRNA tracr and scaffold sequences

| SEQ ID NO. | Nucleotide Sequence |
|---|---|
| 451 | ACAUCUGGCGCGUUUAUUCCAUUACUUUGGAGCCAGUCCCAGCGACUAUGUCGUAUGG |
| | ACGAAGCGCUUAUUUAUCGGAGAGAAACCGAUAAGUAAAACGCAUCAAAG |
| 452 | UACUGGCGCUUUUAUCUCAUUACUUUGAGAGCCAUCACCAGCGACUAUGUCGUAUGGG |
| | UAAAGCGCUUAUUUAUCGGAGAGAAAUCCGAUAAAUAAGAAGCAUCAAAG |
| 453 | ACAUCUGGCGCGUUUAUUCCAUUACUUUGGAGCCAGUCCCAGCGACUAUGUCGUAUGG |
| | ACGAAGCGCUUAUUUAUCGGAGA |
| 454 | ACAUCUGGCGCGUUUAUUCCAUUACUUUGGAGCCAGUCCCAGCGACUAUGUCGUAUGG |
| | ACGAAGCGCUUAUUUAUCGG |
| 455 | UACUGGCGCUUUUAUCUCAUUACUUUGAGAGCCAUCACCAGCGACUAUGUCGUAUGGG |
| | UAAAGCGCUUAUUUAUCGGAGA |
| 456 | UACUGGCGCUUUUAUCUCAUUACUUUGAGAGCCAUCACCAGCGACUAUGUCGUAUGGG |
| | UAAAGCGCUUAUUUAUCGG |
| 457 | GUUUACACACUCCCUCUCAUAGGGU |
| 458 | GUUUACACACUCCCUCUCAUGAGGU |
| 459 | UUUUACAUACCCCCUCUCAUGGGAU |
| 460 | GUUUACACACUCCCUCUCAUGGGGG |
| 461 | CCAGCGACUAUGUCGUAUGG |
| 462 | GCGCUUAUUUAUCGGAGAGAAAUCCGAUAAAUAAGAAGC |
| 463 | GGCGCUUUUAUCUCAUUACUUUGAGAGCCAUCACCAGCGACUAUGUCGUAUGGGUAAA |
| | GCGCUUAUUUAUCGGA |

b. gRNA Domains and their Function

The gRNAs of the systems of the disclosure comprise two segments: a targeting sequence and a protein-binding segment. The targeting segment of a gRNA includes a nucleotide sequence (referred to interchangeably as a spacer, a targeter, or a targeting sequence) that is complementary to (and therefore hybridizes with) a specific sequence (a target site) within the target nucleic acid sequence (e.g., a strand of a double stranded target DNA, a target ssRNA, a target ssDNA, etc.), described more fully below. The targeting sequence of a gRNA is capable of binding to a target nucleic acid sequence, including, in the context of the present disclosure, a coding sequence, a complement of a coding sequence, a non-coding sequence, and to accessory elements. The protein-binding segment (or “activator” or “protein-binding sequence”) of the gRNA interacts with (e.g., binds to) a CasX protein as a complex, forming an RNP (described more fully, below). As used herein, “scaffold” refers to all parts to the guide with the exception of the targeting sequence, which is comprised of several regions, described more fully, below. The properties and characteristics of CasX gRNA, both wild-type and variants, are described in WO2020247882A1, US20220220508A1, and WO2022120095A1, incorporated by reference herein.

In the case of a reference gRNA, the gRNA occurs naturally as a dual guide RNA (dgRNA), wherein the targeter and the activator portions each have a duplex-forming segment that have complementarity with one another and hybridize to one another to form a double stranded duplex (dsRNA duplex for a gRNA). The term “targeter” or “targeter RNA” is used herein to refer to a crRNA-like molecule (crRNA: “CRISPR RNA”) of a CasX dual guide RNA (and therefore of a CasX single guide RNA when the “activator” and the “targeter” are linked together, e.g., by intervening nucleotides). The crRNA has a 5′ region that anneals with the tracrRNA followed by the nucleotides of the targeting sequence. In the case of the gRNA for use in the systems of the disclosure, the scaffolds are designed such that the activator and targeter portions are covalently linked to one another (rather than hybridizing to one another) and comprise a single molecule, and can be referred to as a “single-molecule gRNA,” “single guide RNA”, a “single-molecule guide RNA,” a “one-molecule guide RNA”, or a “sgRNA”. The gRNA variants of the disclosure for use in the systems are all single molecule versions.

Collectively, the assembled gRNAs of the disclosure comprise distinct structured regions, or domains: the RNA triplex, the scaffold stem loop, the extended stem loop, the pseudoknot, and the targeting sequence that is specific for a target nucleic acid and is located on the 3′ end of the gRNA. The RNA triplex, the scaffold stem loop, the pseudoknot and the extended stem loop, together with the unstructured triplex loop that bridges portions of the triplex, together, are referred to as the “scaffold” of the gRNA. In some cases, the scaffold stem further comprises a bubble. In other cases, the scaffold further comprises a triplex loop region. In still other cases, the scaffold further comprises a 5′ unstructured region. In some embodiments, the gRNA scaffolds of the disclosure for use in the systems of the disclosure comprise a scaffold stem loop having the sequence of CCAGCGACUAUGUCGUAGUGG (SEQ ID NO: 542), or a sequence with at least at least 1, 2, 3, 4, or 5 mismatches thereto.

Each of the structured domains contribute to establishing the global RNA fold of the guide and retain functionality of the guide; particularly the ability to properly complex with the CasX protein. For example, the guide scaffold stem interacts with the helical I domain of CasX protein, while residues within the triplex, triplex loop, and pseudoknot stem interact with the OBD of the CasX protein. Together, these interactions confer the ability of the guide to bind and form an RNP with the CasX that retains stability, while the spacer (or targeting sequence) directs and defines the specificity of the RNP for binding a specific sequence of DNA.

Site-specific binding and/or cleavage of a target nucleic acid sequence (e.g., genomic DNA) by the CasX protein can occur at one or more locations (e.g., a sequence of a target nucleic acid) determined by base-pairing complementarity between the targeting sequence of the gRNA and the target nucleic acid sequence. Thus, for example, the gRNA of the disclosure have sequences complementarity to and therefore can hybridize with the target nucleic acid that is adjacent to a sequence complementary to a TC protospacer adjacent motif (PAM) motif or a PAM sequence, such as ATC, CTC, GTC, or TTC. Because the targeting sequence of a guide sequence hybridizes with a sequence of a target nucleic acid sequence, a targeting sequence can be modified by a user to hybridize with a specific target nucleic acid sequence, so long as the location of the PAM sequence is considered. By selection of the targeting sequences of the gRNA, defined regions of the target nucleic acid sequence or sequences bracketing a particular location within the target nucleic acid can be modified or edited using the CasX:gRNA systems described herein. In some embodiments, the targeting sequence of the gRNA has between 15 and 20 consecutive nucleotides. In some embodiments, the targeting sequence has 15, 16, 17, 18, 19, and 20 consecutive nucleotides. In some embodiments, the targeting sequence consists of 20 consecutive nucleotides. In some embodiments, the targeting sequence consists of 19 consecutive nucleotides. In some embodiments, the targeting sequence consists of 18 consecutive nucleotides. In some embodiments, the targeting sequence consists of 17 consecutive nucleotides. In some embodiments, the targeting sequence consists of 16 consecutive nucleotides. In some embodiments, the targeting sequence consists of 15 consecutive nucleotides. By selection of the targeting sequences of the gRNA, defined regions of the target nucleic acid sequence can be modified or edited using the CasX:gRNA systems described herein. In some embodiments, the gRNA and linked targeting sequence exhibit a low degree of off-target effects to the DNA of a cell. As used herein, “off-target effects” refers to effects of unintended cleavage, such as mutations and indel formation, at untargeted genomic sites showing a similar but not an identical sequence compared to the target site (i.e., the sequence complementary to the targeting sequence of the gRNA). In some embodiments, the off-target effects exhibited by the gRNA and linked targeting sequence are less than about 5%, less than about 4%, less than 3%, less than about 2%, less than about 1%, less than about 0.5%, less than 0.1% in cells. In some embodiments, the off-target effects are determined in silico. In some embodiments, the off-target effects are determined in an in vitro cell-free assay. In some embodiments, the off-target effects are determined in a cell-based assay.

In some cases, a gRNA targeting sequence linked to a gRNA scaffold of the disclosure is complementary to and hybridizes with a PCSK9 gene exon selected from the group consisting of exon 1, exon 2, exon 3, exon 4, exon 5, exon 6, exon 7, exon 8, exon 9, exon 10, exon 11, and exon 12. In some embodiments, a gRNA targeting sequence is complementary to and hybridizes with a PCSK9 gene exon selected from the group consisting of PCSK9 exon 2, exon 3, and exon 11. In some embodiments, a gRNA targeting sequence is complementary to and hybridizes with a sequence of a PCSK9 splice-acceptor site of an exon. In some embodiments, a gRNA targeting sequence is complementary to and hybridizes with a sequence of a PCSK9 splice-acceptor site of exon 2, exon 5, exon 6, or exon 11. In some embodiments, a gRNA targeting sequence is complementary to and hybridizes with a sequence of a PCSK9 splice-donor site. In some embodiments, a gRNA targeting sequence is complementary to and hybridizes with a sequence of a PCSK9 splice-donor site of exon 2. In other embodiments, a gRNA targeting sequence hybridizes with a PCSK9 intron. In other embodiments, a gRNA targeting sequence hybridizes with a PCSK9 intron-exon junction. In other embodiments, a gRNA targeting sequence hybridizes with an intergenic region of the PCSK9 gene. In other embodiments, a gRNA targeting sequence hybridizes with a PCSK9 regulatory region. In some cases, the PCSK9 regulatory region is a PCSK9 promoter or enhancer. In some cases, the PCSK9 regulatory region is located 5′ of the PCSK9 transcription start site or 3′ of the PCSK9 transcription start. In some cases, the PCSK9 regulatory region is in an intron of the PCSK9 gene. In other cases, the PCSK9 regulatory region comprises the 5′ UTR of the PCSK9 gene. In still other cases, the PCSK9 regulatory region comprises the 3′ UTR of the PCSK9 gene. In some cases, a gRNA targeting sequence hybridizes with a PCSK9 sequence that is a wild-type sequence. The skilled artisan will understand that particular PCSK9 sequences that hybridize with the PCSK9 targeting sequence of the gRNAs of the disclosure may be wild type, while the PCSK9 gene may contain one or more mutations that affect PCSK9 function. For example, PCSK9 targeting sequences may target wild type sequences that are adjacent, or proximal to, mutations in the PCSK9 gene, or positioned to affect splicing of PCSK9 transcripts, and such targeting sequences may be used to effectively modify the PCSK9 gene.

In some embodiments, the target nucleic acid comprises a PAM sequence located 5′ of the targeting sequence with at least a single nucleotide separating the PAM from the first nucleotide of the targeting sequence. In some embodiments, the PAM is located on the non-targeted strand of the target region, i.e., the strand that is complementary to the target nucleic acid. Representative, but non-limiting examples of targeting sequences to wild-type PCSK9 nucleic acids are presented as SEQ ID NOS: 544-13730, and are shown below as Table 9, representing targeting sequences for PCSK9 target nucleic acid for linkage to the gRNA scaffolds of the disclosure (e.g., gRNA 174, 235, 316, or chemically-modified versions thereof). In some embodiments, the targeting sequence of the gRNA comprises a sequence selected from the group consisting of SEQ ID NOS: 544-13730, or a sequence having at least about 65%, at least about 75%, at least about 85%, at least about 95%, at least about 96%, at least about 97%, at least about 98%, or at least about 99% identity thereto. In some embodiments, the targeting sequence of the gRNA comprises a sequence selected from the group consisting of SEQ ID NOS: 544-13730, wherein the targeting sequence of the gRNA has 1, 2, 3, 4, or 5 nucleotides removed from the 3′ end of the targeting sequence. In some embodiments, the PAM sequence is ATC. In some embodiments, the targeting sequence for an ATC PAM comprises SEQ ID NOS: 4612-7120, or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical to SEQ ID NOS: 4612-7120. In some embodiments, the targeting sequence for an ATC PAM is selected from the group consisting of SEQ ID NOS: 4612-7120. In some embodiments, the PAM sequence is CTC. In some embodiments, the targeting sequence for a CTC PAM comprises SEQ ID NOS: 7121-11390, or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical to SEQ ID NOS: 7121-11390. In some embodiments, the targeting sequence for a CTC PAM is selected from the group consisting of SEQ ID NOS: 7121-11390. In some embodiments, the PAM sequence is GTC. In some embodiments, the targeting sequences for a GTC PAM comprises SEQ ID NOS: 11391-13730, or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical to SEQ ID NOS: 11391-13730. In some embodiments, the targeting sequence for a GTC PAM is selected from the group consisting of SEQ ID NOS: 11391-13730. In some embodiments, the PAM sequence is TTC. In some embodiments, a targeting sequences for a TTC PAM comprises SEQ ID NOS: 544-4611, or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical to SEQ ID NOS: 544-4611. In some embodiments, a targeting sequence for a TTC PAM is selected from the group consisting of SEQ ID NOS: 544-4611.

In some embodiments, the targeting sequence of the gRNA consists of a sequence selected from the group consisting of SEQ ID NOS: 544-665 and 2016, as set forth in Table 10. In some embodiments, the targeting sequence of the gRNA consists of a sequence selected from the group consisting of SEQ ID NOS: 544-665 and 2016, as set forth in Table 10. In some embodiments, the targeting sequence of the gRNA consists of a sequence selected from the group consisting of SEQ ID NOS: 544-559, 583, 619 and 627. In some embodiments, the targeting sequence of the gRNA consists of a sequence of SEQ ID NO: 544. In some embodiments, the targeting sequence of the gRNA consists of a sequence of SEQ ID NO: 545. In some embodiments, the targeting sequence of the gRNA consists of a sequence of SEQ ID NO: 546. In some embodiments, the targeting sequence of the gRNA consists of a sequence of SEQ ID NO: 547. In some embodiments, the targeting sequence of the gRNA consists of a sequence of SEQ ID NO: 548. In some embodiments, the targeting sequence of the gRNA consists of a sequence of SEQ ID NO: 549. In some embodiments, the targeting sequence of the gRNA consists of a sequence of SEQ ID NO: 550. In some embodiments, the targeting sequence of the gRNA consists of a sequence of SEQ ID NO: 551. In some embodiments, the targeting sequence of the gRNA consists of a sequence of SEQ ID NO: 552. In some embodiments, the targeting sequence of the gRNA consists of a sequence of SEQ ID NO: 553. In some embodiments, the targeting sequence of the gRNA consists of a sequence of SEQ ID NO: 554. In some embodiments, the targeting sequence of the gRNA consists of a sequence of SEQ ID NO: 555. In some embodiments, the targeting sequence of the gRNA consists of a sequence of SEQ ID NO: 556. In some embodiments, the targeting sequence of the gRNA consists of a sequence of SEQ ID NO: 557. In some embodiments, the targeting sequence of the gRNA consists of a sequence of SEQ ID NO: 558. In some embodiments, the targeting sequence of the gRNA consists of a sequence of SEQ ID NO: 559. In some embodiments, the targeting sequence of the gRNA consists of a sequence of SEQ ID NO: 583. In some embodiments, the targeting sequence of the gRNA consists of a sequence of SEQ ID NO: 619. In some embodiments, the targeting sequence of the gRNA consists of a sequence of SEQ ID NO: 627. In any of the foregoing, the targeting sequence may have 1, 2, 3, 4, or 5 nucleotides removed from the 3′ end of the targeting sequence.

TABLE 9
RNA Sequences of Exemplary Targeting
Sequences of human PCSK9

| SEQ ID NO: | PAM Sequence |
|---|---|
| 544-4611 | TTC |
| 4612-7120 | ATC |
| 7121-11390 | CTC |
| 11391-13730 | GTC |

TABLE 10
RNA Sequences of Exemplary Targeting
Sequences of human PCSK9

| SEQ ID NO: | PAM Sequence |
|---|---|
| 544-665 and 2016 | TTC |

c. gRNA Modifications

In another aspect, the disclosure relates to gRNA for use in the gene-editing systems of the disclosure, which comprise one or more modifications relative to a reference gRNA from which it was derived. In some embodiments, a gRNA variant comprises one or more nucleotide substitutions, insertions, deletions, or swapped or replaced domains relative to a gRNA sequence of the disclosure that improve a characteristic relative to the reference gRNA. Exemplary regions for modifications and swapped regions or domains include the RNA triplex, the pseudoknot, the scaffold stem loop, and the extended stem loop. In some embodiments, the gRNA variant comprises at least a first swapped region from a different gRNA, resulting in a chimeric gRNA. A representative example of such a chimeric gRNA is guide 316 (SEQ ID NO: 466), in which the extended stem loop of gRNA scaffold 235 is replaced with the extended stem loop of gRNA scaffold 174, wherein the resulting 316 variant retains the ability to form an RNP with a CasX protein and exhibits an improved functional characteristic compared to the parent 235, when assessed in an in vitro or in vivo assay under comparable conditions.

All gRNAs that have one or more improved functions, characteristics, or add one or more new functions when the gRNA scaffold variant is compared to a gRNA scaffold from which it was derived, while retaining the functional properties of being able to complex with the CasX protein and guide the CasX ribonucleoprotein holo complex to the target nucleic acid, are envisaged as within the scope of the disclosure. In some embodiments, the gRNA has an improved characteristic selected from the group consisting of increased editing activity, increased pseudoknot stem stability, increased triplex region stability, increased scaffold stem stability, extended stem stability, reduced off-target folding intermediates, and increased binding affinity to a CasX protein, or any combination thereof. In some cases of the foregoing, the improved characteristic is assessed in an in vitro assay, including the assays of the Examples. In other cases of the foregoing, the improved characteristic is assessed in vivo.

In other embodiments, the disclosure provides gRNA variant scaffolds having improved manufacturability compared to the gRNA scaffold from which it was derived. In a particular embodiment, the 316 gRNA scaffold has a shorter sequence compared to the 235 scaffold from which it was derived. For example, the shorter sequence length of the 316 scaffold confers the improvements of a higher fidelity in the ability to create the guide synthetically with the correct and complete sequence, as well as an enhanced ability to be successfully incorporated into an LNP. The 316 variant is described more fully, below.

Table 11 provides exemplary gRNA variant scaffold sequences of the disclosure that are utilized as gRNA scaffolds or for the generation of the gRNAs for use in the CasX:gRNA systems of the disclosure. In some embodiments, the gRNA variant scaffold comprises any one of the sequences listed in Table 11 (SEQ ID NOS: 464-466), or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto, wherein the gRNA variant retains the ability to form an RNP with a CasX of the disclosure. In other embodiments, the gRNA variant scaffold for use in the CasX:gRNA systems consists of any one of the sequences listed in Table 11, wherein the gRNA variant retains the ability to form an RNP with a CasX of the disclosure. It will be understood that in those embodiments wherein a vector comprises a DNA encoding sequence for a gRNA, that thymine (T) bases can be substituted for the uracil (U) bases of any of the gRNA sequence embodiments described herein.

TABLE 11
Exemplary gRNA Scaffold Sequences

| SEQ ID | Scaffold | |
|---|---|---|
| NO: | variant ID | Nucleotide sequence |
| 464 | 174 | ACUGGCGCUUUUAUCUGAUUACUUUGAGAGCCAUCACCAGCGACUAUGUCG |
| | | UAGUGGGUAAAGCUCCCUCUUCGGAGGGAGCAUCAAAG |
| 465 | 235 | ACUGGCGCUUCUAUCUGAUUACUCUGAGCGCCAUCACCAGCGACUAUGUCG |
| | | UAGUGGGUAAAGCCGCUUACGGACUUCGGUCCGUAAGAGGCAUCAGAG |
| 466 | 316 | ACUGGCGCUUCUAUCUGAUUACUCUGAGCGCCAUCACCAGCGACUAUGUCG |
| | | UAGUGGGUAAAGCUCCCUCUUCGGAGGGAGCAUCAGAG |

Additional gRNA variants contemplated for use in the systems of the disclosure are selected from the group consisting of SEQ ID NOS: 467-541, and the chemically modified gRNA selected from the group consisting of SEQ ID NOS: 13749-13777. The skilled artisan will appreciate that SEQ ID NOS: 13749-13777 include the targeting sequences as undefined nucleotides, and that these undefined nucleotides can be any of the targeting sequences disclosed herein, with the indicated chemical modifications. In some embodiments, a chemically modified scaffold of a gRNA comprises a sequence of SEQ ID NOS: 13749-13777, without the 20 undefined nucleotides at the 3′ end. Additional gRNA variants, which are CpG depleted, are encoded by the sequences of SEQ ID NOS: 14065-14068.

d. gRNA Scaffold 316

Guide scaffolds can be made by several methods, including recombinantly or by solid-phase RNA synthesis. However, the length of the scaffold can affect the manufacturability when using solid-phase RNA synthesis, with longer lengths resulting in increased manufacturing costs, decreased purity and yield, and higher rates of synthesis failures. For use in lipid nanoparticle (LNP) formulations, solid-phase RNA synthesis of the scaffold is preferred to generate the quantities needed for commercial development. While previous experiments had identified gRNA scaffold 235 as having enhanced properties relative to gRNA scaffold 174, its increased length (in nucleotides) rendered its use for LNP formulations problematic due to synthetic manufacturing constraints. Accordingly, alternative sequences were sought. In some embodiments, the disclosure provides a gRNA wherein the gRNA scaffold and linked targeting sequence has a sequence less than about 115 nucleotides, less than about 110 nucleotides, or less than about 100 nucleotides.

In one embodiment, a gRNA scaffold was designed wherein the scaffold 235 sequence was modified by a domain swap in which the extended stem loop of scaffold 174 replaced the extended stem loop of the 235 scaffold, resulting in the chimeric gRNA scaffold 316, having the sequence ACUGGCGCUUCUAUCUGAUUACUCUGAGCGCCAUCACCAGCGACUAUGUCGUAGUG GGUAAAGCUCCCUCUUCGGAGGGAGCAUCAGAG (SEQ ID NO: 466), having 89 nucleotides, compared with the 99 nucleotides of gRNA scaffold 235. The resulting 316 scaffold had the further advantage in that the extended stem loop does not contain CpG motifs; an enhanced property conferring reduced potential to elicit an immune response. In some embodiments, the disclosure provides gRNA 316 variants that are chemically-modified, described below.

e. Chemically-Modified gRNAs

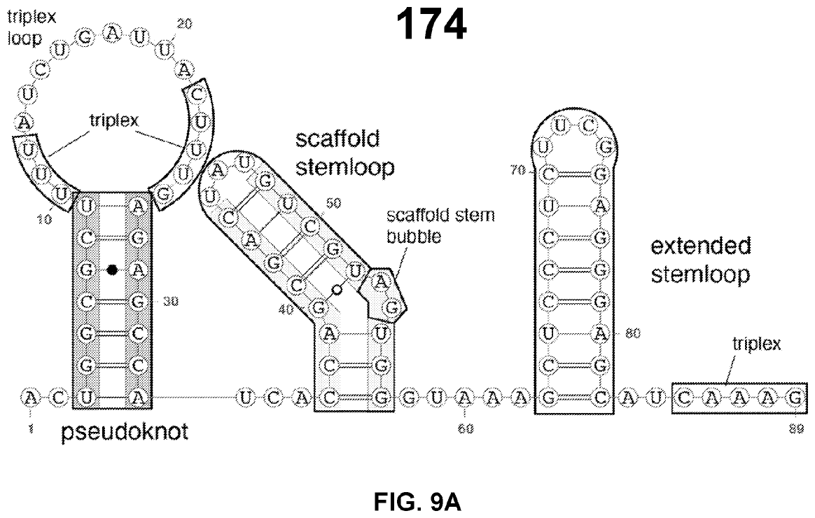

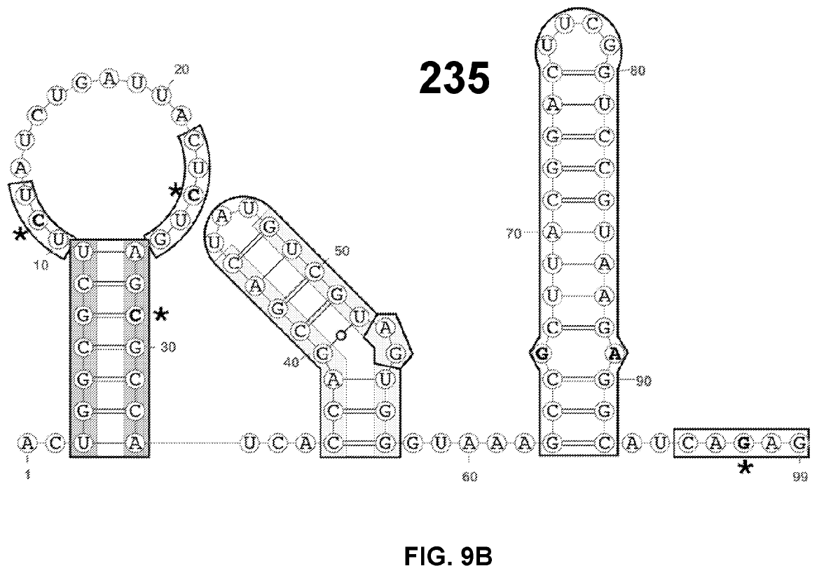

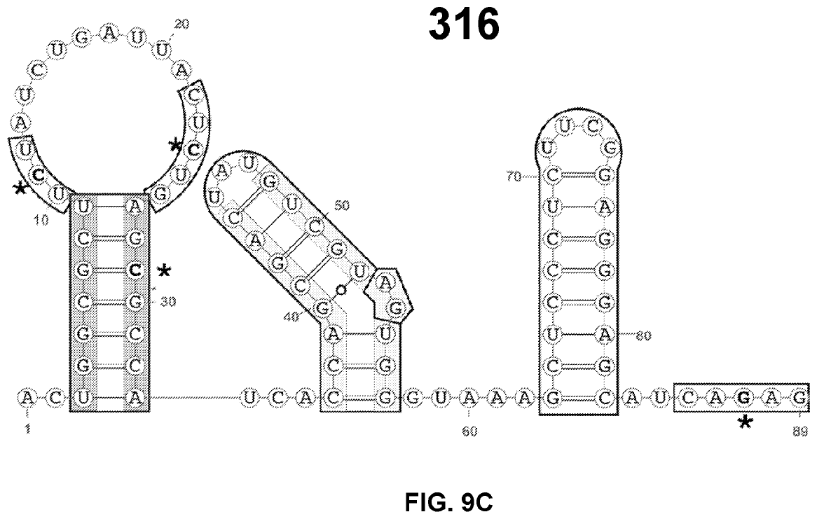

In some embodiments, the gRNAs have one or more chemical modifications. In some embodiments, the one or more chemical modifications comprise the addition of a 2′O-methyl group to one or more nucleotides of the sequence. In some embodiments, one or more nucleotides on each terminal end of the gRNA are modified by an addition of a 2′O-methyl group. In some embodiments, the one or more chemical modifications comprise substitution of a phosphorothioate bond between two or more nucleosides of the sequence; i.e., the phosphorothioate bond replaces the phosphate bond. In some embodiments, the one or more chemical modifications comprise a substitution of phosphorothioate bonds between two or more nucleosides on each terminal end of the gRNA. In some embodiments, the first 1, 2, or 3 nucleosides of the 5′ end of the scaffold (i.e., A, C, and U in the case of gRNA 174, 235, and 316) are modified by the addition of a 2′O-methyl group and each of the modified nucleosides is linked to the adjoining nucleoside by a phosphorothioate bond. Similarly, the last 1, 2, or 3 nucleotides of the 3′ end of the targeting sequence linked to the 3′ end of the scaffold are similarly modified. In some embodiments, the disclosure provides gRNA with chemical modifications selected from the group consisting of the sequences of SEQ ID NOS: 13749-13757; 13759-13767; 13769-13777, as set forth in Table 16, or a sequence having at least about 70%, at least about 80%, at least about 90%, at least about 95%, at least about 96%, at least about 97%, at least about 98%, at least about 99% sequence identity thereto. In some embodiments, the gRNA with chemical modifications comprises a scaffold of SEQ ID NOS: 13749-13757, 13759-13767, 13769-13777, i.e., a sequence of SEQ ID NOS: 13749-13757, 13759-13767, 13769-13777 without the spacer represented in the foregoing sequences as undefined nucleotides. In some embodiments, the gRNA with chemical modifications comprises a scaffold of SEQ ID NO: 13759, without the spacer represented in the foregoing sequences as undefined nucleotides (it being understood that for utilization in the systems of the disclosure, the non-targeting 20 nucleotides at the 3′ end are replaced with a targeting sequence complementary to the PCSK9 target nucleic acid to be modified). In some embodiments, the gRNA with chemical modifications comprises a scaffold of SEQ ID NO: 13769, without the spacer represented in the foregoing sequences as undefined nucleotides (it being understood that for utilization in the systems of the disclosure, the non-targeting 20 nucleotides at the 3′ end are replaced with a targeting sequence complementary to the PCSK9 target nucleic acid to be modified). A schematic of the structure of gRNA variants 174, 235, and 316 are shown in FIGS. 9A-9C, respectively. In some embodiments, the gRNAs with chemical modifications exhibit improved stability, including enhanced metabolic stability in a cell, compared to an otherwise equivalent gRNA without chemical modifications. In some embodiments, the chemically-modified gRNA exhibit at least about 10%, at least about 20%, at least about 30%, at least about 40%, at least about 50%, at least about 60% less susceptibility to degradation by cellular RNase compared to an unmodified gRNA.

f. Complex Formation with CasX Protein

Upon delivery or expression of the components of the system in a target cell, the gRNA variant is capable of complexing as an RNP with a CasX protein and binding to the target nucleic acid of the PCSK9 gene. In some embodiments, a gRNA variant has an improved ability to form an RNP complex with an engineered CasX protein when compared to a reference gRNA or another gRNA variant from which it was derived. Improving ribonucleoprotein complex formation may, in some embodiments, improve the efficiency with which functional RNPs are assembled. In some embodiments, greater than 90%, greater than 93%, greater than 95%, greater than 96%, greater than 97%, greater than 98% or greater than 99% of RNPs comprising a gRNA variant and its targeting sequence are competent for gene editing or modification of a target nucleic acid.

### V. Polynucleotides and Vectors

In another aspect, the present disclosure relates to polynucleotides encoding the Class 2, Type V nucleases and gRNAs that have utility in the editing of the PCSK9 gene. Additionally, the disclosure provides vectors comprising polynucleotides encoding the engineered Class 2, Type V nucleases and the gRNAs described herein. In some cases, the Class 2, Type V nucleases are CasX proteins, and the gRNAs are CasX gRNAs. In some cases, the vectors are utilized for the expression and recovery of the engineered CasX and gRNA components of the gene editing pair. In other cases, the vectors are utilized for the delivery of the encoding polynucleotides to target cells for the editing of the target nucleic acid, as described more fully, below. In some embodiments, sequences encoding the CasX and gRNA are encoded on the same vector. In some embodiments, sequences encoding the CasX and a gRNA are encoded on different vectors. Suitable vectors are described, for example, in WO2022120095A1 and WO2020247882A1, incorporated by reference herein. As described in WO2022120095A1 and WO2020247882A1, depending on the host/vector system utilized, any of a number of suitable transcription and translation control elements, including constitutive and inducible promoters, transcription enhancer elements, transcription terminators, etc. may be used in the expression vector.

In some embodiments, the disclosure provides polynucleotide sequences encoding the engineered CasX of any of the embodiments described herein, including the engineered CasX proteins of SEQ ID NOS: 4-342, 14126-14286, and 14352-14354 or sequences having at least about 50%, at least about 60%, at least about 70%, at least about 80%, at least about 90%, at least about 95%, at least about 96%, at least about 97%, at least about 98%, or at least about 99% sequence identity thereto. In some embodiments, the disclosure provides a polynucleotide sequence encoding an engineered CasX selected from the group consisting of SEQ ID NOS: 4-7, or a sequence having at least about 70%, at least about 80%, at least about 90%, at least about 95%, at least about 96%, at least about 97%, at least about 98%, or at least about 99% sequence identity thereto. In some embodiments, the polynucleotide comprises a sequence encoding an engineered CasX protein selected from the group consisting of SEQ ID NOS: 4-342, 14126-14286, and 14352-14354. In some embodiments, the polynucleotide comprises a sequence encoding an engineered CasX protein selected from the group consisting of SEQ ID NOS: 5-342, 14126-14286, and 14352-14354. In some embodiments, the polynucleotide comprises a sequence encoding an engineered CasX protein selected from the group consisting of SEQ ID NOS: 45-342, 14126-14286, and 14352-14354. In some embodiments, the polynucleotide comprises a sequence encoding an engineered CasX protein selected from the group consisting of SEQ ID NOS: 14126-14286 and 14352-14354. In some embodiments, the disclosure provides an mRNA sequence encoding an engineered CasX, wherein the mRNA comprises a sequence selected from the group consisting of SEQ ID NOS: 13740-13743, or a sequence having at least about 70%, at least about 80%, at least about 90%, at least about 95%, at least about 96%, at least about 97%, at least about 98%, or at least about 99% sequence identity thereto. In some embodiments, the disclosure provides an mRNA sequence encoding an engineered CasX selected from the group consisting of SEQ ID NOS: 4-7, or a sequence having at least about 70%, at least about 80%, at least about 90%, at least about 95%, at least about 96%, at least about 97%, at least about 98%, or at least about 99% sequence identity thereto. In some embodiments, the disclosure provides a polynucleotide sequence encoding a gRNA variant of any of the embodiments described herein. In some embodiments, the disclosure provides polynucleotides encoding a gRNA scaffold sequence of any one of SEQ ID NOS: 464-541, or a sequence having at least about 70%, at least about 80%, at least about 90%, at least about 95%, at least about 96%, at least about 97%, at least about 98%, at least about 99% sequence identity thereto, wherein the expressed gRNA variant retains the ability to form an RNP with a CasX. In some embodiments, the polynucleotide encodes a gRNA scaffold sequence of any one of SEQ ID NOS: 464-541. In some embodiments, the polynucleotide encodes a gRNA scaffold sequence of any one of SEQ ID NOS: 465-466 and 513-541. In some embodiments, the polynucleotide encodes a gRNA scaffold sequence of any one of SEQ ID NOS: 464-466. In some embodiments, the polynucleotide encodes a gRNA comprising a targeting sequence selected from the group consisting of SEQ ID NOS: 544-13730. In other embodiments, the disclosure provides polynucleotides encoding gRNAs comprising targeting sequences selected from the group consisting of SEQ ID NOS: 544-665 and 2016, or sequences having at least about 65%, at least about 70%, at least about, 75%, at least about, 80%, at least about, 85%, at least about 90%, or at least about 95% identity thereto. In other embodiments, the disclosure provides polynucleotides encoding gRNAs comprising targeting sequences selected from the group consisting of SEQ ID NOS: 544-665 and 2016.

In some embodiments, the disclosure provides systems comprising an mRNA sequence encoding an engineered CasX comprising the sequence of SEQ ID NO: 13741, or a sequence having at least about 70%, at least about 80%, at least about 90%, at least about 95%, at least about 96%, at least about 97%, at least about 98%, or at least about 99% sequence identity thereto, and a gRNA comprising a scaffold sequence of SEQ ID NO: 466 having a linked targeting sequence selected from the group consisting of SEQ ID NOS: 544-559, 583, 619 and 627. In some cases, the gRNA sequence is chemically modified. In alternative embodiments embodiment of the foregoing, the gRNA comprises a sequence selected from the group consisting of SEQ ID NOS: 13769-13777, or a sequence having at least about 65%, at least about 70%, at least about 75%, at least about 80%, at least about 85%, at least about 90%, or at least about 95% identity thereto. The skilled artisan will appreciate that SEQ ID NOS: 13769-13777 include the targeting sequences as undefined nucleotides, and that these undefined nucleotides can be any of the targeting sequences disclosed herein, with the indicated chemical modifications.

In some embodiments, the disclosure relates to methods to produce polynucleotide sequences encoding the engineered CasX and/or the gRNA of any of the embodiments described herein, including variants thereof, as well as methods to express the proteins and RNAs encoded by the polynucleotide sequences. In general, the methods include producing a polynucleotide sequence coding for the CasX or the gRNA of any of the embodiments described herein and incorporating the encoding polynucleotide into an expression vector. In some embodiments, the vector is designed for transduction of cells for modification of the PCSK9 target nucleic acid. Such vectors can include a retroviral vector, a lentiviral vector, an adenoviral vector, an adeno-associated viral (AAV) vector, a herpes simplex virus (HSV) vector, a plasmid, a minicircle, a nanoplasmid, a DNA vector, and an RNA vector. In other embodiments, the expression vector is designed for production of CasX, mRNA encoding CasX, or gRNA in either a cell-free system or in a host cell. For production of the encoded CasX or the gRNA of any of the embodiments described herein in a host cell, the methods include transforming an appropriate host cell with an expression vector comprising the encoding polynucleotide, and culturing the host cell under conditions causing or permitting the resulting CasX or the gRNA to be expressed or transcribed in the transformed host cell, thereby producing the CasX or the gRNA, which are recovered by methods described herein (e.g., in the Examples, below), or by standard purification methods known in the art. Standard recombinant techniques in molecular biology can be used to make the polynucleotides and expression vectors of the present disclosure.

In accordance with the disclosure, nucleic acid sequences that encode the engineered CasX or the gRNA of any of the embodiments described herein are used to generate recombinant DNA molecules that direct the expression in appropriate host cells. Several cloning strategies are suitable for performing the present disclosure, many of which are used to generate a construct that comprises a gene coding for a composition of the present disclosure, or its complement. In some embodiments, the cloning strategy is used to create a gene that encodes a construct that comprises nucleotides encoding the engineered CasX or the gRNA that is used to transform a host cell for expression of the composition.

In one approach, a construct is first prepared containing the DNA sequence encoding an engineered CasX or a gRNA. Exemplary methods for the preparation of such constructs are described in the Examples. The construct is then used to create an expression vector suitable for transforming a host cell, such as a prokaryotic or eukaryotic host cell for the expression and recovery of the protein construct, in the case of the CasX, or the gRNA. In some embodiments, the host cell is an E. coli. In other embodiments, the host cell is a eukaryotic cell. The eukaryotic host cell can be selected from Baby Hamster Kidney fibroblast (BHK) cells, human embryonic kidney 293 (HEK293), human embryonic kidney 293T (HEK293T), NSO cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS), HeLa, Chinese hamster ovary (CHO), yeast cells, or other eukaryotic cells known in the art suitable for the production of recombinant products. Exemplary methods for the creation of expression vectors, the transformation of host cells and the expression and recovery of the engineered CasX or the gRNA are described in the Examples.

The gene encoding the engineered CasX or the gRNA construct can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate sequences of polynucleotides encoding the various components into a gene of a desired sequence. Genes encoding polypeptide compositions are assembled from oligonucleotides using standard techniques of gene synthesis.

In some embodiments, the nucleotide sequence encoding a CasX protein is modified to optimize its expression. This type of modification can entail a mutation of an encoding nucleotide sequence to mimic the codon preferences of the intended host organism or cell while encoding the same protein. Thus, the codons can be changed, but the encoded protein remains unchanged. For example, if the intended target cell of the CasX protein was a human cell, a human codon-modified CasX-encoding nucleotide sequence could be used. As another non-limiting example, if the intended host cell were a mouse cell, then a mouse codon-modified CasX-encoding nucleotide sequence could be generated. The gene design can be performed using algorithms that reflect codon usage and amino acid composition appropriate for the host cell utilized in the production of the engineered CasX or the gRNA. In one method of the disclosure, a library of polynucleotides encoding the components of the constructs is created and then assembled, as described above. The resulting genes are then assembled and the resulting genes used to transform a host cell and produce and recover the engineered CasX or the gRNA compositions for evaluation of its properties or for use in the modification of the PCSK9 target nucleic acid, as described herein.

In some embodiments, a nucleotide sequence encoding a gRNA is operably linked to a control element, e.g., a transcriptional control element, such as a promoter. In some embodiments, a nucleotide sequence encoding a CasX protein is operably linked to a control element, e.g., a transcriptional control element, such as a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells, e.g., hepatocytes or a liver sinusoidal endothelial cell.

Non-limiting examples of Pol II promoters operably linked to the polynucleotide encoding the engineered CasX of the disclosure include, but are not limited to EF-1alpha, EF-1alpha core promoter, Jens Tornoe (JeT), promoters from cytomegalovirus (CMV), CMV immediate early (CMVIE), CMV enhancer, herpes simplex virus (HSV) thymidine kinase, early and late simian virus 40 (SV40), the SV40 enhancer, long terminal repeats (LTRs) from retrovirus, mouse metallothionein-I, adenovirus major late promoter (Ad MLP), CMV promoter full-length promoter, the minimal CMV promoter, the chicken β-actin promoter (CBA), CBA hybrid (CBh), chicken β-actin promoter with cytomegalovirus enhancer (CB7), chicken beta-Actin promoter and rabbit beta-Globin splice acceptor site fusion (CAG), the rous sarcoma virus (RSV) promoter, the HIV-Ltr promoter, the hPGK promoter, the HSV TK promoter, a 7SK promoter, the Mini-TK promoter, the human synapsin I (SYN) promoter which confers neuron-specific expression, beta-actin promoter, super core promoter 1 (SCP1), the Mecp2 promoter for selective expression in neurons, the minimal IL-2 promoter, the Rous sarcoma virus enhancer/promoter (single), the spleen focus-forming virus long terminal repeat (LTR) promoter, the TBG promoter, promoter from the human thyroxine-binding globulin gene (Liver specific), the PGK promoter, the human ubiquitin C promoter (UBC), the UCOE promoter (Promoter of HNRPA2B1-CBX3), the synthetic CAG promoter, the Histone H2 promoter, the Histone H3 promoter, the U1a1 small nuclear RNA promoter (226 nt), the U1a1 small nuclear RNA promoter (226 nt), the U1b2 small nuclear RNA promoter (246 nt) 26, the GUSB promoter, the CBh promoter, rhodopsin (Rho) promoter, silencing-prone spleen focus forming virus (SFFV) promoter, a human H1 promoter (H1), a POLI promoter, the TTR minimal enhancer/promoter, the b-kinesin promoter, mouse mammary tumor virus long terminal repeat (LTR) promoter, the human eukaryotic initiation factor 4A (EIF4A1) promoter, the ROSA26 promoter, the glyceraldehyde 3-phosphate dehydrogenase (GAPDH) promoter, tRNA promoters, and truncated versions and sequence variants of the foregoing. In a particular embodiment, the Pol II promoter is EF-1alpha, wherein the promoter enhances transfection efficiency, the transgene transcription or expression of the CRISPR nuclease, the proportion of expression-positive clones and the copy number of the episomal vector in long-term culture.

Non-limiting examples of Pol III promoters operably linked to the polynucleotide encoding the gRNA variants of the disclosure include, but are not limited to U6, mini U6, U6 truncated promoters, 7SK, and H1 variants, BiH1 (Bidrectional H1 promoter), BiU6, Bi7SK, BiH1 (Bidirectional U6, 7SK, and H1 promoters), gorilla U6, rhesus U6, human 7SK, human H1 promoters, and truncated versions and sequence variants thereof. In the foregoing embodiment, the pol III promoter enhances the transcription of the gRNA. In a particular embodiment, the Pol III promoter is U6, wherein the promoter enhances expression of the CRISPR gRNA. In another particular embodiment, the promoter linked to the gene encoding the tropism factor is CMV promoter. Experimental details and data for the use of such promoters are provided in the Examples.

Selection of the appropriate vector and promoter is well within the level of ordinary skill in the art, as it related to controlling expression. The expression vector may also contain a ribosome binding site for translation initiation, and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to the CasX protein, thus resulting in a chimeric CasX protein that are used for purification or detection.

Recombinant expression vectors of the disclosure can also comprise elements that facilitate robust expression of the proteins and the gRNAs of the disclosure. For example, recombinant expression vectors can include one or more of a polyadenylation signal (poly(A)), an intronic sequence or a post-transcriptional regulatory element such as a woodchuck hepatitis post-transcriptional regulatory element (WPRE). Exemplary poly(A) sequences include hGH poly(A) signal (short), HSV TK poly(A) signal, synthetic polyadenylation signals, SV40 poly(A) signal, β-globin poly(A) signal and the like, for example SEQ ID NO: 13988. A person of ordinary skill in the art will be able to select suitable elements to include in the recombinant expression vectors described herein.

The polynucleotides encoding the engineered CasX or the gRNA sequences can be individually cloned into an expression vector. Selection of the appropriate vector and promoter is well within the level of ordinary skill in the art, as it relates to controlling expression, e.g., for modifying expression of the CasX protein. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, FLAG tag, fluorescent protein, etc.) that can be fused to the CasX protein, thus resulting in a chimeric CasX protein that are used for purification or detection.

The nucleic acid sequence is inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available. The vector may, for example, be in the form of a plasmid, cosmid, viral particle, or phage that may conveniently be subjected to recombinant DNA procedures, and the choice of vector will often depend on the host cell into which it is to be introduced. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. Once introduced into a suitable host cell, expression of the CasX can be determined using any nucleic acid or protein assay known in the art. For example, the presence of transcribed mRNA of the engineered CasX can be detected and/or quantified by conventional hybridization assays (e.g., Northern blot analysis), amplification procedures (e.g. RT-PCR), SAGE (U.S. Pat. No. 5,695,937), and array-based technologies (see e.g., U.S. Pat. Nos. 5,405,783, 5,412,087 and 5,445,934), using probes complementary to any region of CasX polynucleotide.

In some embodiments, a vector is created for the transcription of the engineered CasX gene and expression and recovery of the resulting encoding mRNA. In some embodiments, the mRNA is generated by in vitro transcription (IVT) using a PCR product or linearized plasmid DNA template and a T7 RNA polymerase, wherein the plasmid contains a T7 promoter. If using a PCR product, DNA sequences encoding candidate mRNAs will be cloned into a plasmid containing a T7 promoter, wherein the plasmid DNA template will be linearized and then used to perform IVT reactions for expression of the mRNA. Exemplary methods for the generation of such vectors and the production and recovery of the mRNA are provided in the Examples, below.

In some embodiments, a recombinant expression vector of the present disclosure encoding the CasX:gRNA system is a recombinant adeno-associated virus (AAV) vector. AAV is a small (20 nm), nonpathogenic virus that is useful in treating human diseases in situations that employ a viral vector for delivery to a cell such as a eukaryotic cell, either in vivo or ex vivo for cells to be prepared for administering to a subject. An “AAV virus” or “AAV viral particle” refers to a viral particle composed of at least one AAV capsid protein (preferably by all of the capsid proteins of a wild-type AAV) and an encapsidated polynucleotide. If the particle additionally comprises a heterologous polynucleotide (i.e., a polynucleotide other than a wild-type AAV genome to be delivered to a mammalian cell), it is typically referred to as “rAAV”. An exemplary heterologous polynucleotide is a polynucleotide encoding a CasX protein and one or more gRNA of any of the embodiments described herein. A construct is generated, for example a construct encoding any of the CasX proteins and/or CasX gRNA embodiments as described herein, and is flanked with AAV inverted terminal repeat (ITR) sequences, thereby enabling packaging of the AAV vector into an AAV viral particle.

An “AAV” vector may refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on capsid protein reactivity with defined antisera, e.g., there are many known serotypes of primate AAVs. In some embodiments, the AAV vector is selected from AAV1, AAV2, AAV3, AAV4, AAV5, AAV6, AAV7, AAV8, AAV9, AAV10, AAV11, AAV12, AAV 9.45, AAV 9.61, AAV-Rh74 (Rhesus macaque-derived AAV), and AAVRh10, and modified capsids of these serotypes. For example, serotype AAV-2 is used to refer to an AAV which contains capsid proteins encoded from the cap gene of AAV-2 and a genome containing 5′ and 3′ ITR sequences from the same AAV-2 serotype. Pseudotyped AAV refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the ITR serotype. Pseudotyped recombinant AAV (rAAV) are produced using standard techniques described in the art. As used herein, for example, rAAV1 may be used to refer an AAV having both capsid proteins and 5′-3′ ITRs from the same serotype or it may refer to an AAV having capsid proteins from serotype 1 and 5′-3′ ITRs from a different AAV serotype, e.g., AAV serotype 2. For each example illustrated herein the description of the vector design and production describes the serotype of the capsid and 5′-3′ ITR sequences. By “adeno-associated virus inverted terminal repeats” or “AAV ITRs” is meant the art recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, for example Kotin, R. M. (1994) Human Gene Therapy 5:793-801; Berns, K. I. “Parvoviridae and their Replication” in Fundamental Virology, 2^{nd} Edition, (B. N. Fields and D. M. Knipe, eds.). As used herein, an AAV ITR need not have the wild-type nucleotide sequence depicted, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV6, AAV7, AAV8, AAV9, AAV10, AAV-Rh74, and AAVRh10, and modified capsids of these serotypes. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Use of AAV serotypes for integration of heterologous sequences into a host cell is known in the art (see, e.g., WO2018195555A1 and US20180258424A1, incorporated by reference herein). In one particular embodiment, the ITRs are derived from serotype AAV1. In a particular embodiment, the ITR regions flanking the transgene of the embodiments are derived from AAV2. In some embodiments, the 5′ ITR of the transgene of the AAV constructs of the disclosure has the sequence CCTGCAGGCAGCTGCGCGCTCGCTCGCTCACTGAGGCCGCCCGGGCGTCGGGCGACCT TTGGTCGCCCGGCCTCAGTGAGCGAGCGAGCGCGCAGAGAGGGAGTGGCCAACTCCAT CACTAGGGGTTCCT (SEQ ID NO: 13778). In some embodiments, the 3′ ITR of the transgene of the AAV constructs of the disclosure has the sequence AGGAACCCCTAGTGATGGAGTTGGCCACTCCCTCTCTGCGCGCTCGCTCGCTCACTGAG GCCGGGCGACCAAAGGTCGCCCGACGCCCGGGCTTTGCCCGGGCGGCCTCAGTGAGCG AGCGAGCGCGCAGCTGCCTGCAGG (SEQ ID NO: 13779). In other embodiments, the ITR sequences are modified to remove unmethylated CpG motifs to reduce immunogenic responses. In particular, CpG dinucleotide motifs (CpG PAMPs) in AAV vectors are immunostimulatory because of their high degree of hypomethylation, relative to mammalian CpG motifs, which have a high degree of methylation. In one embodiment, the modified AAV 2 ITR sequences are modified to remove CpG motifs, such that the 5′ITR has the sequence of TGCTCACTCACTCACTCACTGAGGCCTGCAGAGCAAAGCTCTGCAGTCTGGGGACCTTT GGTCCCCAGGCCTCAGTGAGTGAGTGAGTGAGCAGAGAGGGAGTGGCCAACTCCATCA CTAGGGGTTCCT (SEQ ID NO: 13780) and the 3′ ITR sequence is the sequence

(SEQ ID NO: 13781)
TCTGCTCACTCACTCACTCACTGAGGCCTGCAGAGCAAAGCTCTGCAGTCTGGGGACCT
TTGGTCCCCAGGCCTCAGTGAGTGAGTGAGTGAGCAGAGAGGGAGTGGCCAACTCCAT
CACTAGGGGTTCCT.

In some embodiments, AAV capsids utilized for delivery of the encoding sequences for the CasX and gRNA to a host cell can be derived from any of several AAV serotypes, including without limitation, AAV1, AAV2, AAV3, AAV4, AAV5, AAV6, AAV7, AAV8, AAV9, AAV10, AAV11, AAV12, AAV 9.45, AAV 9.61, AAV-Rh74 (Rhesus macaque-derived AAV), and AAVRh10, and the AAV ITRs are derived from AAV serotype 2. In some embodiments, the AAV vector and the regulatory sequences are selected so that the total size of the vector is 5 kb or less, permitting packaging within the AAV capsid. Representative constructs are described in the Examples, as well as in WO2022125843A1, incorporated by reference herein.

In order to eliminate any integrative capacity of the virus, recombinant AAV vectors remove rep and cap from the DNA of the viral genome and a three-plasmid system can be utilized to transfect a suitable host packaging cell. To produce such vectors, the desired transgenes, together with promoters to drive transcription of the transgenes and any enhancer elements, are inserted between the ITRs, and the rep and cap genes are provided in trans in a second plasmid. A third plasmid, providing helper genes such as adenovirus E4, E2a and VA genes, is also used. All three plasmids are then transfected into an appropriate packaging cell using known techniques, such as by transfection. Alternatively, the host cell genome may comprise stably integrated Rep and Cap genes. Suitable packaging cell lines are known to one of ordinary skill in the art. See for example, www.cellbiolabs.com/aav-expression-and-packaging.

Packaging cells are typically used to form virus particles. Packaging cells can be eukaryotic cells, for example mammalian cells. The packaging cell can be selected from the group consisting of Baby Hamster Kidney fibroblast (BHK) cells, human embryonic kidney 293 (HEK293), human embryonic kidney 293T (HEK293T), NS0 cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS), HeLa, and Chinese hamster ovary (CHO) cells, or other eukaryotic cells known in the art suitable for the production of recombinant AAV. A number of transfection techniques are generally known in the art; see, e.g., Sambrook et al. (1989) Molecular Cloning, a laboratory manual, Cold Spring Harbor Laboratories, New York. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles.

In some embodiments, host cells transfected with the above-described AAV expression vectors are rendered capable of providing AAV helper functions in order to replicate and encapsulate the nucleotide sequences flanked by the AAV ITRs to produce rAAV viral particles. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs (open reading frames), encoding the rep and cap coding regions, or functional homologues thereof. Accessory functions can be introduced into and then expressed in host cells using methods known to those of skill in the art. Commonly, accessory functions are provided by infection of the host cells with an unrelated helper virus. In some embodiments, accessory functions are provided using an accessory function vector. Depending on the host/vector system utilized, any of a number of suitable transcription and translation control elements, including constitutive and inducible promoters, transcription enhancer elements, transcription terminators, etc., may be used in the expression vector.

### VI. Particles for Delivery of CasX: GRNA Systems

In another aspect, the present disclosure provides particle compositions for delivery of the CasX:gRNA systems to cells or to subjects for the modification of the PCSK9 gene. In some embodiments, the disclosure provides synthetic nanoparticles that encapsulate gRNA variants and mRNA encoding engineered CasX of any of the embodiments described herein. In some embodiments, materials for the creation of biodegradable polymeric nanoparticles (PNP) include polylactide, poly(lactic-co-glycolic acid) (PLGA), poly(ethyl cyanoacrylate), poly(butyl cyanoacrylate), poly(isobutyl cyanoacrylate), and poly(isohexyl cyanoacrylate), polyglutamic acid (PGA), poly(ε-caprolactone) (PCL), cyclodextrin, and natural polymers for instance chitosan, albumin, gelatin, and alginate are the most utilized polymers for the synthesis of PNP (Production and clinical development of nanoparticles for gene delivery. Molecular Therapy-Methods & Clinical Development 3:16023; doi: 10.1038 (2016)). In some embodiments, the disclosure provides virus-like particles for delivery of the engineered CasX and gRNA variants (see, WO2021113772A1, incorporated by reference herein). In other embodiments, the disclosure provides lipid nanoparticles that encapsulate gRNA variants and mRNA encoding engineered CasX of any of the embodiments described herein, described more fully, below.

a. Lipid Nanoparticles (LNP)

In another aspect, the present disclosure provides lipid nanoparticles (LNP) for delivery of the CasX:gRNA systems to cells or to subjects for the modification of the PCSK9 gene. In some embodiments, the LNPs of the disclosure are liver tissue-specific, have excellent biocompatibility, and can deliver the CasX:gRNA systems with high efficiency, and thus can be usefully used for the modification of the PCSK9 gene.

In their native forms, nucleic acid polymers are unstable in biological fluids and cannot penetrate into the cytoplasm of target cells, thus requiring delivery systems. Lipid nanoparticles (LNP) have proven useful for both the protection and delivery of nucleic acids to tissues and cells. Furthermore, the use of mRNA in LNPs to encode the CRISPR nuclease eliminates the possibility of undesirable genome integration compared to DNA vectors. Moreover, mRNA efficiently transfects both mitotic and non-mitotic cells, as it does not require to enter into the nucleus since it exerts its function in the cytoplasmic compartment. LNPs as a delivery platform offers the additional advantage of being able to co-formulate both the mRNA encoding the nuclease and the gRNA into single LNP particles.

Accordingly, in various embodiments, the disclosure encompasses lipid nanoparticles and compositions that may be used for a variety of purposes, including the delivery of encapsulated or associated (e.g., complexed) therapeutic agents such as nucleic acids to cells, both in vitro and in vivo. In certain embodiments, the disclosure encompasses methods of treating or preventing diseases or disorders in a subject in need thereof by contacting the subject with a lipid nanoparticle that encapsulates or is associated with a suitable therapeutic agent complexed through various physical, chemical or electrostatic interactions between one or more of the lipid components used in the compositions to make LNPs. In some embodiments, the suitable therapeutic agent comprises a CasX:gRNA system as described herein.

In certain embodiments, the lipid nanoparticles are useful for the delivery of nucleic acids, including, e.g., the mRNA encoding the CasX of the disclosure, including the sequences of SEQ ID NOS: 4-7 as set forth in Table 1 and the gRNA embodiments of the disclosure, including the sequences of SEQ ID NOS: 464-466 and 13748-13777. In some embodiments, the gRNA comprises a sequence of SEQ ID NOS: 465-466 or 513-541. In some embodiments, the present disclosure provides LNP in which the gRNA and mRNA encoding the engineered CasX are incorporated into single LNP particles. In other embodiments, the present disclosure provides LNP in which the gRNA and mRNA encoding the engineered CasX are incorporated into separate LNP particles, which can be formulated together in varying ratios for administration. In some embodiments, the mRNA for incorporation into the LNP of the disclosure encode any of the engineered CasX described herein, including the sequences selected from the group consisting of SEQ ID NOS: 4-7, 9-342, 14126-14286, and 14352-14354. In some embodiments, the mRNA for incorporation into the LNP of the disclosure encode an engineered CasX comprising a sequence selected from the group consisting of SEQ ID NOS: 5-7, 9-342, 14126-14286, and 14352-14354. In some embodiments, the mRNA for incorporation into the LNP of the disclosure encode an engineered CasX comprising a sequence selected from the group consisting of SEQ ID NOS: 45-342, 14126-14286, and 14352-14354. In some embodiments, the mRNA for incorporation into the LNP of the disclosure encode an engineered CasX comprising a sequence selected from the group consisting of SEQ ID NOS: 14126-14286 and 14352-14354. In some embodiments, the gRNA for use in the LNP comprises the sequence of SEQ ID NO: 466 or the chemically modified sequence of SEQ ID NO: 13769.

The lipid nanoparticles and systems of certain embodiments of the disclosure may be used to induce expression of a desired protein both in vitro and in vivo by contacting cells with a lipid nanoparticle comprising one or more novel cationic lipids described herein, wherein the lipid nanoparticle encapsulates or is associated with a nucleic acid that is expressed to produce the desired protein (e.g., a messenger RNA encoding the CasX protein). In some embodiments, the lipid nanoparticles and systems may be used to decrease the expression of the PCKS9 target gene both in vitro and in vivo by contacting cells with a lipid nanoparticle comprising one or more novel cationic lipids described herein, wherein the lipid nanoparticle encapsulates or is associated with nucleic acids of the CasX:gRNA system that reduces target gene expression. The lipid nanoparticles and systems of embodiments of the disclosure may also be used for co-delivery of different nucleic acids (e.g., mRNA and plasmid DNA) separately or in combination, such as may be useful to provide an effect requiring colocalization of different nucleic acids (e.g., mRNA encoding for a suitable gene modifying enzyme and gRNA for targeting of the target nucleic acid).

In some embodiments, LNPs and LNP compositions described herein include at least one cationic lipid, at least one conjugated lipid, at least one steroid or derivative thereof, at least one additional lipid, or any combination thereof. Alternatively, the lipid compositions of the disclosure can include an ionizable lipid, such as an ionizable cationic lipid, a helper lipid (usually a phospholipid), cholesterol, and a polyethylene glycol-lipid conjugate (PEG-lipid) to improve the colloidal stability in biological environments by, for example, reducing a specific absorption of plasma proteins and forming a hydration layer over the nanoparticles. Such lipid compositions can be formulated at typical mole ratios of 50:10:37-39:1.5-2.5 or 20-50:8-65:25-40:1-2.5, with variations made to adjust individual properties.

The LNPs and LNP compositions of the present disclosure are configured to protect and deliver an encapsulated payload of the systems of the disclosure to tissues and cells, both in vitro and in vivo. Various embodiments of the LNPs and LNP compositions of the present disclosure are described in further detail herein.

### Cationic Lipid

In some aspects, the LNPs and LNP compositions of the present disclosure include at least one cationic lipid. The term “cationic lipid,” refers to a lipid species that has a net positive charge. In some embodiments, the cationic lipid is an ionizable cationic lipid that has a net positive charge at a selected pH, such as physiological pH. In some embodiments, the ionizable cationic lipid has a pKa less than about 7 such that the LNPs and LNP compositions achieve efficient encapsulation of the payload at a relatively low pH. In some embodiments, the cationic lipid has a pKa of about 5 to about 8, about 5.5 to about 7.5, about 6 to about 7, or about 6.5 to about 7. In some embodiments, the cationic lipid may be protonated at a pH below the pKa of the cationic lipid, and it may be substantially neutral at a pH over the pKa. The LNPs and LNP compositions may be safely delivered to a target organ (for example, the liver, lung, heart, spleen, as well as to tumors) and/or cell (hepatocyte, LSEC, cardiac cell, cancer cell, etc.) in vivo, and after endocytosis, exhibit a positive charge to release the encapsulated payload through electrostatic interaction with an anionic protein of the endosome membrane.

Early formulations of LNP utilizing permanently cationic lipids resulted in LNPs with positive surface charge that proved toxic in vivo, plus were rapidly cleared by phagocytic cells. By changing to ionizable cationic lipids bearing tertiary amines, especially those with pKa<7, results in LNP achieving efficient encapsulation of nucleic acid polymers at low pH by interacting electrostatically with the negative charges of the phosphate backbone of mRNA, that also result in largely neutral systems at physiological pH values, thus alleviating problems associated with permanently-charged cationic lipids.

As used herein, “ionizable lipid” means an amine-containing lipid which can be easily protonated, and, for example, it may be a lipid of which charge state changes depending on the surrounding pH. The ionizable lipid may be protonated (positively charged) at a pH below the pKa of a cationic lipid, and it may be substantially neutral at a pH over the pKa. In one example, the LNP may comprise a protonated ionizable lipid and/or an ionizable lipid showing neutrality. In some embodiments, the LNP has a pKa of about 5 to about 8, about 5.5 to about 7.5, about 6 to about 7, or about 6.5 to about 7. The pKa of the LNP is important for in vivo stability and release of the nucleic acid payload of the LNP in the target cell or organ. In some embodiments, the LNP having the foregoing pKa ranges may be safely delivered to a target organ (for example, the liver, lung, heart, spleen, as well as to tumors) and/or target cell (hepatocyte, LSEC, cardiac cell, cancer cell, etc.) in vivo, and after endocytosis, exhibit a positive charge to release the encapsulated payload through electrostatic interaction with an anionic protein of the endosome membrane.

The ionizable lipid is an ionizable compound having characteristics similar to lipids generally, and when the pH of the formulation buffer is kept below its pKa, the ionizable lipid through electrostatic interaction with a nucleic acid (for example, an mRNA of the disclosure), may play a role of encapsulating the nucleic acid payloads within the LNP with high efficiency.

According to the type of the amine and the tail group comprised in the ionizable lipid, (i) the nucleic acid encapsulation efficiency, (ii) PDI (polydispersity index). and/or (iii) the nucleic acid delivery efficiency to tissue and/or cells constituting an organ (for example, hepatocytes or liver sinusoidal endothelial cells in the liver) of the LNP may be different. In certain embodiments, the ionizable lipid is an ionizable cationic lipid, and comprises from about 46 mol % to about 66 mol % of the total lipid present in the particle.

The LNP comprising an ionizable lipid comprising an amine may have one or more kinds of the following characteristics: (1) the ability toencapsulate a nucleic acid with high efficiency; (2) uniform size of prepared particles (or having a low PDI value); and/or (3) excellent nucleic acid delivery efficiency to organs such as liver, lung, heart, spleen, bone marrow, as well as to tumors, and/or cells constituting such organs (for example, hepatocytes, LSEC, cardiac cells, cancer cells, etc.).

In particular embodiments, the cationic lipid form plays a crucial role both in nucleic acid encapsulation through electrostatic interactions and intracellular release by disrupting endosomal membranes. The nucleic acid payloads are encapsulated within the LNP by the ionic interactions they form with the positively charged cationic lipid. Non-limiting examples of cationic lipid components utilized in the LNP of the disclosure are selected from DLin-MC3-DMA (heptatriaconta-6,9,28,31-tetraen-19-yl4-(dimethylamino)butanoate), DLin-KC2-DMA (2,2-dilinoleyl-4-(2-dimethylaminoethyl)-[1,3]-dioxolane), and TNT (1,3,5-triazinane-2,4,6-trione) and TT (N1,N3,N5-tris(2-aminoethyl)benzene-1,3,5-tricarboxamide). Non-limiting examples of helper lipids utilized in the LNP of the disclosure are selected from DSPC (1,2-distearoyl-sn-glycero-3-phosphocholine), POPC (2-Oleoyl-1-palmitoyl-sn-glycero-3-phosphocholine) and DOPE (1,2-Dioleoyl-sn-glycero-3-phosphoethanolamine), 1,2-dioleoyl-sn-glycero-3-phospho-(1′-rac-glycerol) DOPG, 1,2-Dimyristoyl-sn-glycero-3-phosphoethanolamine (DMPE), 1,2-dilauroyl-sn-glycero-3-phosphocholine (DLPC), sphingolipid, and ceramide. Cholesterol and PEG-DMG ((R)-2,3-bis(octadecyloxy)propyl-1-(methoxy polyethylene glycol 2000)carbamate), PEG-DSG (1,2-Distearoyl-rac-glycero-3-methylpolyoxyethylene glycol 2000), or DSPE-PEG2k (1,2-distearoyl-sn-glycero-3-phosphoethanolamine-N-[amino (polyethylene glycol)-2000]), are components utilized in the LNP of the disclosure for the stability, circulation, and size of the LNP.

In some embodiments, the cationic lipid in the LNP of the disclosure comprises a tertiary amine. In some embodiments, the tertiary amine includes alkyl chains connected to N of the tertiary amine with ether linkages. In some embodiments, the alkyl chains comprise C12-C30 alkyl chains having 0 to 3 double bonds. In some embodiments, the alkyl chains comprise C16-C22 alkyl chains. In some embodiments, the alkyl chains comprise C18 alkyl chains. A number of cationic lipids and related analogs have been described in U.S. Patent Publication Nos. 20060083780, 20060240554, 20110117125, 20190336608, 20190381180 and 20200121809; U.S. Pat. Nos. 5,208,036; 5,264,618; 5,279,833; 5,283,185; 5,753,613; 5,785,992; 9,738,593; 10,106,490; 10,166,298; 10,221,127; and 11,219,634; and PCT Publication No. WO 96/10390, the disclosures of which are herein incorporated by reference in their entirety.

In some embodiments, the cationic lipid in the LNP of the disclosure may comprise, for example, one or more ionizable cationic lipids wherein the ionizable cationic lipid is a dialkyl lipid. In other embodiments, the ionizable cationic lipid is a trialkyl lipid.

In some embodiments, the cationic lipid in the LNP of the disclosure is selected from 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3 -dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), and any combination of the forgoing.

In some embodiments, the cationic lipid in the LNP of the disclosure is selected from heptatriaconta-6,9,28,31-tetraen-19-yl4-(dimethylamino)butanoate (DLin-MC3-DMA), 2,2-dilinoleyl-4-(2-dimethylaminoethyl)-[1,3]-dioxolane (DLin-KC2-DMA), (1,3,5-triazinane-2,4,6-trione) (TNT), N1,N3,N5-tris(2-aminoethyl)benzene-1,3,5-tricarboxamide (TT), and any combination of the forgoing.

In some embodiments, the N/P ratio (nitrogen from the cationic/ionizable lipid and phosphate from the nucleic acid) in the LNP of the disclosure is in the range of is about 3:1 to 7:1, or about 4:1 to 6:1, or is 3:1, or is 4:1, or is 5:1, or is 6:1, or is 7:1.

### Conjugated Lipid

In some embodiments, the LNPs and LNP compositions of the present disclosure include at least one conjugated lipid. In some embodiments, the conjugated lipid may be selected from a polyethyleneglycol (PEG)-lipid conjugate, a polyamide (ATTA)-lipid conjugate, a cationic-polymer-lipid conjugate (CPL), and any combination of the foregoing. In some cases, conjugated lipids can inhibit aggregation of the LNPs of the disclosure.

In some embodiments, the conjugated lipid of the LNP of the disclosure comprises a pegylated lipid. The terms “polyethyleneglycol (PEG)-lipid conjugate,” “pegylated lipid” “lipid-PEG conjugate”, “lipid-PEG”, “PEG-lipid”, “PEG-lipid”, or “lipid-PEG” are used interchangeably herein and refer to a lipid attached to a polyethylene glycol (PEG) polymer which is a hydrophilic polymer. The pegylated lipid contributes to the stability of the LNPs and LNP compositions and reduces aggregation of the LNPs.

As the PEG-lipid can form the surface lipid, the size of the LNP can be readily varied by varying the proportion of surface (PEG) lipid to the core (ionizable cationic) lipids. In some embodiments, the PEG-lipid of the LNP of the disclosure can be varied from ~1 to 5 mol % to modify particle properties such as size, stability, and circulation time.

The lipid-PEG conjugate contributes to the particle stability in serum of the nanoparticle within the LNP, and plays a role of preventing aggregation between nanoparticles. In addition, the lipid-PEG conjugate may protect nucleic acids, such as mRNAs encoding the CasX proteins of the disclosure, or gRNAs of the disclosure, from degrading enzymes during in vivo delivery of the nucleic acids and enhance the stability of the nucleic acids in vivo and increase the half-life of the delivered nucleic acids encapsulated in the nanoparticle. Examples of PEG-lipid conjugates include, but are not limited to, PEG-DAG conjugates, PEG-DAA conjugates, and mixtures thereof. In certain embodiments, the PEG-lipid conjugate is selected from the group consisting of a PEG-diacylglycerol (PEG-DAG) conjugate, a PEG-dialkyloxypropyl (PEG-DAA) conjugate, a PEG-phospholipid conjugate, a PEG-ceramide (PEG-Cer) conjugate, and a mixture thereof.

In some embodiments, the pegylated lipid of the LNP of the disclosure is selected from a PEG-ceramide, a PEG-diacylglycerol, a PEG-dialkyloxypropyl, a PEG-dialkoxypropylcarbamate, a PEG-phosphatidylethanoloamine, a PEG-phospholipid, a PEG-succinate diacylglycerol, and any combination of the foregoing.

In some embodiments, the pegylated lipid of the LNP of the disclosure is a PEG-dialkyloxypropyl. In some embodiments, the pegylated lipid is selected from PEG-didecyloxypropyl (C10), PEG-dilauryloxypropyl (C12), PEG-dimyristyloxypropyl (C14), PEG-dipalmityloxypropyl (C16), PEG-distearyloxypropyl (C18), and any combination of the foregoing.

In other embodiments, the lipid-PEG conjugate of the LNP of the disclosure may be PEG bound to phospholipid such as phosphatidylethanolamine (PEG-PE), PEG conjugated to ceramide (PEG-CER, ceramide-PEG conjugate, ceramide-PEG, cholesterol or PEG conjugated to derivative thereof, PEG-c-DOMG, PEG-DMG, PEG-DLPE, PEG-DMPE, PEG-DPPC, PEG-DSPE (DSPE-PEG), and a mixture thereof, and for example, may be C16-PEG2000 ceramide (N-palmitoyl-sphingosine-1-{succinyl [methoxy(polyethylene glycol)2000]}), DMG-PEG 2000, 14:0 PEG2000 PE.

In some embodiments, the pegylated lipid of the LNP of the disclosure is selected from 1-(monomethoxy-polyethyleneglycol)-2,3-dimyristoylglycerol, 4-O-(2′,3′-di(tetradecanoyloxy)propyl-1-O-(ω-methoxy(polyethoxy)ethyl)butanedioate (PEG-S-DMG), ω-methoxy(polyethoxy)ethyl-N-(2,3-di(tetradecanoxy)propyl)carbamate, 2,3-di(tetradecanoxy)propyl-N-(ω-methoxy(polyethoxy)ethyl)carbamate, and any combination of the foregoing.

In some embodiments, the pegylated lipid of the LNP of the disclosure is selected from mPEG2000-1,2-di-O-alkyl-sn3-carbomoylglyceride (PEG-C-DOMG), 1-[8′-(1,2-dimyristoyl-3-propanoxy)-carboxamido-3′,6′-dioxaoctanyl]carbamoyl-w-methyl-poly(ethylene glycol) (2 KPEG-DMG), and any combination of the foregoing.

In some embodiments, the PEG is directly attached to the lipid of the pegylated lipid. In other embodiments, the PEG is attached to the lipid of the pegylated lipid by a linker moiety selected from an ester-free linker moiety or an ester-containing linker moiety. Non-limiting examples of the ester-free linker moiety include amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH2CH2C(O)—), succinamidyl (—NHC(O)CH2CH2C(O)NH—), ether, disulfide and combinations thereof. For example, the linker may contain a carbamate linker moiety and an amido linker moiety. Non-limiting examples of the ester-containing linker moiety include carbonate (—OC(O)O—), succinoyl, phosphate ester (—O—(O)POH—O—), sulfonate ester, and combinations thereof.

The PEG moiety of the pegylated lipid of the LNP of the disclosure described herein may have an average molecular weight ranging from about 550 daltons to about 10,000 daltons. In certain embodiments, the PEG moiety has an average molecular weight of from about 750 daltons to about 5,000 daltons, about 1,000 daltons to about 4,000 daltons, about 1,500 daltons to about 3,000 daltons, about 750 daltons to about 3,000 daltons, or about 1750 daltons to about 2,000 daltons.

In some embodiments, the conjugated lipid (e.g., pegylated lipid) comprises from about 1 mol % to about 60 mol %, from about 2 mol % to about 50 mol %, from about 5 mol % to about 40 mol %, or from about 5 mol % to about 20 mol % of the total lipid present in the LNPs and/or LNP compositions. In certain embodiments, the conjugated lipid comprises from about 0.5 mol % to about 3 mol % of the total lipid present in the particle.

In additional embodiments, the conjugated lipid (e.g., pegylated lipid) of the LNP of the disclosure comprises at least about 1, 2, 5, 10, 15, 20, 25, 30, 35, 40, 45, 50, 55, or 60 mol %, or an intermediate range of any of the foregoing, of the total lipid present in the LNPs and/or LNP compositions.

For the lipid in the lipid-PEG conjugate of the LNP of the disclosure, any lipid capable of binding to polyethyleneglycol may be used without limitation, and the phospholipid and/or cholesterol which are other elements of the LNP may be also used. In some embodiments, the lipid in the lipid-PEG conjugate may be ceramide, dimyristoylglycerol (DMG), succinoyl-diacylglycerol (s-DAG), distearoylphosphatidylcholine (DSPC), distearoylphosphatidylethanolamine (DSPE), or cholesterol, but not limited thereto.

In the lipid-PEG conjugate of the LNP of the disclosure, the PEG may be directly conjugated to the lipid or linked to the lipid via a linker moiety. Any linker moiety suitable for binding PEG to the lipid may be used, and for example, includes an ester-free linker moiety and an ester-containing linker moiety. The ester-free linker moiety includes not only amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH2CH2C(O)—), succinamidyl (—NHC(O)CH2CH2C(O)NH—), ether, disulfide but also combinations thereof (for example, a linker containing both a carbamate linker moiety and an amido linker moiety), but not limited thereto. The ester-containing linker moiety includes for example, carbonate (—OC(O)O—), succinoyl, phosphate ester (—O—(O)POH—O—), sulfonate ester, and combinations thereof, but not limited thereto.

### Steroids

In some embodiments, the LNPs and LNP compositions of the present disclosure include at least one steroid or derivative thereof. In some embodiments, the steroid comprises cholesterol. In some embodiments, the LNPs and LNP compositions comprise a cholesterol derivative selected from cholestanol, cholestanone, cholestenone, coprostanol, cholesteryl-2′-hydroxyethyl ether, cholesteryl-4′-hydroxybutyl ether, and any combination of the foregoing.

In some embodiments, the steroid (e.g., cholesterol) of the LNP of the disclosure comprises from about 1 mol % to about 60 mol %, from about 2 mol % to about 50 mol %, from about 5 mol % to about 40 mol %, or from about 5 mol % to about 20 mol % of the total lipid present in the LNPs and/or LNP compositions. In other embodiments, the steroid (e.g., cholesterol) of the LNP of the disclosure comprises at least about 1, 2, 5, 10, 15, 20, 25, 30, 35, 40, 45, 50, 55, or 60 mol %, or an intermediate range of any of the foregoing, of the total lipid present in the LNPs and/or LNP compositions.

### Additional Lipid

In some embodiments, the LNPs and LNP compositions of the present disclosure include at least one additional lipid. In some embodiments, the additional lipid is non-cationic lipid selected from an anionic lipid, a neutral lipid, or both. In some embodiments, the additional lipid comprises at least one phospholipid. In some embodiments, the phospholipid is selected from an anionic phospholipid, a neutral phospholipid, or both. The phospholipid of the elements of the LNPs and LNP compositions can play a role in covering and protecting a core of the LNP formed by interaction of the cationic lipid and nucleic acid in the LNP, and may facilitate cell membrane permeation and endosomal escape during intracellular delivery of the nucleic acid by binding to the phospholipid bilayer of a target cell. A phospholipid which can promote fusion of the LNP to a cell may include without limitation, any of the phospholipids selected from the group described below.

In some embodiments, the LNPs and LNP compositions comprise at least one phospholipid selected from, but not limited to, dipalmitoyl-phosphatidylcholine (DPPC), distearoyl-phosphatidylcholine (DSPC), dioleoyl-phosphatidylethanolamine (DOPE), dioleoyl-phosphatidylcholine (DOPC), dioleoyl-phosphatidylglycerol (DOPG), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dipalmitoyl-phosphatidylglycerol (DPPG), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), phosphatidylethanolamine (PE), 1,2-dioleoyl-sn-glycero-3-phosphoethanolamine, 1-palmitoyl-2-oleoyl-sn-glycero-3-phosphocholine (POPC), 1,2-dioleoyl-sn-glycero-3-[phospho-L-serine] (DOPS), 1,2-dioleoyl-sn-glycero-3-[phospho-L-serine], and any combination of the foregoing. In one example, the LNP comprising DOPE may be effective in mRNA delivery (excellent drug delivery efficacy).

In some embodiments, the additional lipid (e.g., phospholipid) of the LNP of the disclosure comprises from about 1 mol % to about 60 mol %, from about 2 mol % to about 50 mol %, from about 5 mol % to about 40 mol %, or from about 5 mol % to about 20 mol % of the total lipid present in the LNPs and/or LNP compositions. In other embodiments, the additional lipid (e.g., phospholipid) of the LNP of the disclosure comprises at least about 1, 2, 5, 10, 15, 20, 25, 30, 35, 40, 45, 50, 55, or 60 mol %, or an intermediate range of any of the foregoing, of the total lipid present in the LNPs and/or LNP compositions.

It will be appreciated that the total lipid present in the LNPs and/or LNP compositions comprises the combination of the cationic lipid or ionizable cationic lipid, the conjugated lipid, (e.g., pegylated lipid), the steroid (e.g., cholesterol), and the additional lipid (e.g., phospholipid).

The LNPs and/or LNP compositions may be prepared by dissolving the total lipids (or a portion thereof) in an organic solvent (e.g., ethanol) followed by mixing through a micromixer with the payload (e.g., nucleic acids of the systems) dissolved in an acidic buffer (e.g., pH 4). At this pH the cationic lipid is positively charged and interacts with the negatively-charged nucleic acid polymers. The resulting nanostructures containing the nucleic acids are then converted to neutral LNPs when dialyzed against a neutral buffer which may then be followed by removal of the organic solvent (e.g., ethanol) and exchange the LNPs into physiologically relevant buffer. The LNPs and/or LNP compositions thus formed have a distinct electron-dense nanostructured core where the cationic lipids are organized into inverted micelles around the encapsulated payload, as opposed to traditional bilayer liposomal structures. In another embodiment, the LNP may form a bleb-like structure with nucleic acids in aqueous pockets along the non-electron dense lipid core.

b. Lipid Nanoparticle Properties

In some embodiments, the LNPs and/or LNP compositions comprise from about 50 mol % to about 85 mol % of the cationic lipid or ionizable cationic lipid, from about 0.5 mol % to about 10 mol % of the conjugated lipid, (e.g., pegylated lipid), from about 0.5 mol % to about 10 mol % of the steroid (e.g., cholesterol) and from about 5 mol % to about 50 mol % of the additional lipid (e.g., phospholipid). In some embodiments, the LNPs and/or LNP compositions comprise from about 50 mol % to about 85 mol % of the cationic lipid or ionizable cationic lipid, from about 0.5 mol % to about 5 mol % of the conjugated lipid, (e.g., pegylated lipid), from about 0.5 mol % to about 5 mol % of the steroid (e.g., cholesterol) and from about 5 mol % to about 20 mol % of the additional lipid (e.g., phospholipid).

In some embodiments, the LNPs and/or LNP compositions of the disclosure comprise cationic lipid: additional lipid (e.g., phospholipid): steroid (e.g., cholesterol): conjugated lipid, (e.g., pegylated lipid) at a molar ratio of 20 to 50:10 to 30:30 to 60:0.5 to 5, at a molar ratio of 25 to 45:10 to 25:40 to 50:0.5 to 3, at a molar ratio of 25 to 45:10 to 20:40 to 55:0.5 to 3, or at a molar ratio of 25 to 45:10 to 20:40 to 55:1.0 to 1.5.

In some embodiments, the LNPs and/or LNP compositions of the disclosure have a total lipid: payload ratio (mass/mass) of from about 1 to about 100. In some embodiments, the total lipid: payload ratio is about 1 to about 50, from about 2 to about 25, from about 3 to about 20, from about 4 to about 15, or from about 5 to about 10. In some embodiments, the total lipid: payload ratio is about 5 to about 15, e.g., about 5, 6, 7, 8, 9, 10, 11, 12, 13, 14, 15, or an intermediate range of any of the foregoing.

In certain embodiments, the LNPs of the disclosure comprise a total lipid: nucleic acid mass ratio of from about 5:1 to about 15:1. In some embodiments, the weight ratio of the cationic lipid and nucleic acid comprised in the LNP may be 1 to 20:1, 1 to 15:1, 1 to 10:1, 5 to 20:1, 5 to 15:1, 5 to 10:1, 7.5 to 20:1, 7.5 to 15:1, or 7.5 to 10:1.

In some embodiments, the LNP of the disclosure may comprise the cationic lipid of 20 to 50 parts by weight, the phospholipid of 10 to 30 parts by weight, cholesterol of 20 to 60 parts by weight (or 20 to 60 parts by weight), and lipid-PEG conjugate of 0.1 to 10 parts by weight (or 0.25 to 10 parts by weight, 0.5 to 5 parts by weight). Alternatively, the LNP may comprise the cationic lipid of 20 to 50% by weight, phospholipid of 10 to 30% by weight, cholesterol of 20 to 60% by weight (or 30 to 60% by weight), and lipid-PEG conjugate of 0.1 to 10% by weight (or 0.25 to 10% by weight, 0.5 to 5% by weight) based on the total nanoparticle weight. As a further alternative, the LNP may comprise the cationic lipid of 25 to 50% by weight, phospholipid of 10 to 20% by weight, cholesterol of 35 to 55% by weight, and lipid-PEG conjugate of 0.1 to 10% by weight (or 0.25 to 10% by weight, 0.5 to 5% by weight), based on the total nanoparticle weight.

In some embodiments, the LNPs of the present disclosure have a mean diameter of from about 20 to 200 nm, 20 to 180 nm, 20 to 170 nm, 20 to 150 nm, 20 to 120 nm, 20 to 100 nm, 20 to 90 nm, 30 to 200 nm, 30 to 180 nm, 30 to 170 nm, 30 to 150 nm, 30 to 120 nm, 30 to 100 nm, 30 to 90 nm, 40 to 200 nm, 40 to 180 nm, 40 to 170 nm, 40 to 150 nm, 40 to 120 nm, 40 to 100 nm, 40 to 90 nm, 40 to 80 nm, 40 to 70 nm, 50 to 200 nm, 50 to 180 nm, 50 to 170 nm, 50 to 150 nm, 50 to 120 nm, 50 to 100 nm, 50 to 90 nm, 60 to 200 nm, 60 to 180 nm, 60 to 170 nm, 60 to 150 nm, 60 to 120 nm, 60 to 100 nm, 60 to 90 nm, 70 to 200 nm, 70 to 180 nm, 70 to 170 nm, 70 to 150 nm, 70 to 120 nm, 70 to 100 nm, 70 to 90 nm, 80 to 200 nm, 80 to 180 nm, 80 to 170 nm, 80 to 150 nm, 80 to 120 nm, 80 to 100 nm, 80 to 90 nm, 90 to 200 nm, 90 to 180 nm, 90 to 170 nm, 90 to 150 nm, 90 to 120 nm, or 90 to 100 nm, or an intermediate range of any of the foregoing.

In some embodiments, the LNPs and/or LNP compositions of the disclosure have a positive charge at acidic pH and may encapsulate the payload (e.g., therapeutic agent) through electrostatic charges produced by negative charges of the payload (e.g., therapeutic agent). The term “encapsulation,” refers to the mixture of lipids surrounding and embedding the payload (e.g., therapeutic agent) at physiological conditions, forming the LNPs. The term “encapsulation efficiency,” as used herein is the amount of payload (e.g., therapeutic agent) encapsulated by the LNPs divided by the total amount of payload (e.g., therapeutic agent) used to load the payload (e.g., therapeutic agent) into the LNPs. The encapsulation efficiency of the LNPs and/or LNP compositions may be 70% or more, 75% or more, 80% or more, 85% or more, 90% or more, 91% or more, 92% or more, 94% or more, or 95% or more. In other embodiments, the encapsulation efficiency of the LNPs and/or LNP compositions is about 80% to 99%, about 85% to 98%, about 88% to 95%, about 90% to 95%, or the payload (e.g., nucleic acids of the systems) may be fully encapsulated within the lipid portion of the LNPs compositions, and thereby protected from enzymatic degradation. In some embodiments, the payload (e.g., therapeutic agent) is not substantially degraded after exposure of the LNPs and/or LNP compositions to a nuclease at 37° C. for at least about 20, 30, 45, or 60 minutes or at least about 2, 3, 4, 5, 6, 7, 8, 9, 10, 12, 14, 16, 18, 20, 22, 24, 26, 28, 30, 32, 34, or 36 hours. In some embodiments, the payload (e.g., nucleic acids of the systems) is complexed with the lipid portion of the LNPs and/or LNP compositions. The LNPs and/or LNP compositions of the present disclosure are non-toxic to mammals such as humans.

The term “fully encapsulated” indicates that the payload (e.g., the nucleic acids of the system) in the LNPs and/or LNP compositions is not significantly degraded after exposure to conditions that significantly degrade free DNA, RNA, or protein. In a fully encapsulated system, less than about 25%, more preferably less than about 10%, and most preferably less than about 5% of the payload (e.g., nucleic acids of the system) in the LNPs and/or LNP compositions is degraded by conditions that would degrade 100% of a non-encapsulated payload. “Fully encapsulated” also indicates that the LNPs and/or LNP compositions are serum-stable, and do not decompose into their component parts upon in vivo administration.

In some embodiments, the amount of the LNPs and/or LNP compositions having the payload (e.g., therapeutic agent), encapsulated therein is from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, %, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, or an intermediate range of any of the foregoing.

In some embodiments, the amount of the payload (e.g., the nucleic acids), encapsulated within the LNPs and/or LNP compositions is from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, %, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, or an intermediate range of any of the foregoing.

In some embodiments, the nucleic acids of the disclosure, such as the mRNA encoding the CasX fusion protein, and/or the gRNA, may be provided in a solution to be mixed with a lipid solution such that the nucleic acids may be encapsulated in the lipid nanoparticles. A suitable nucleic acid solution may be any aqueous solution containing the nucleic acid to be encapsulated at various concentrations. For example, a suitable nucleic acid solution may contain the nucleic acid (or nucleic acids) at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.25 mg/ml, 1.5 mg/ml, 1.75 mg/ml, or 2.0 mg/ml. In some embodiments, the nucleic acid comprises an mRNA encoding an CasX, and a suitable mRNA solution may contain the mRNA at a concentration ranging from about 0.01-2.0 mg/ml, 0.01-1.5 mg/ml, 0.01-1.25 mg/ml, 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml. In some embodiments, a suitable mRNA solution may contain an mRNA at a concentration up to about 5.0 mg/ml, 4.0 mg/ml, 3.0 mg/ml, 2.0 mg/ml, 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.05 mg/ml, 0.04 mg/ml, 0.03 mg/ml, 0.02 mg/ml, 0.01 mg/ml, or 0.05 mg/ml. In some embodiments, a suitable gRNA solution may contain an gRNA at a concentration up to about 5.0 mg/ml, 4.0 mg/ml, 3.0 mg/ml, 2.0 mg/ml, 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.05 mg/ml, 0.04 mg/ml, 0.03 mg/ml, 0.02 mg/ml, 0.01 mg/ml, or 0.05 mg/ml

In some embodiments, the LNP may have an average diameter of 20 nm to 200 nm, 20 to 180 nm, 20 nm to 170 nm, 20 nm to 150 nm, 20 nm to 120 nm, 20 nm to 100 nm, 20 nm to 90 nm, 30 nm to 200 nm, 30 to 180 nm, 30 nm to 170 nm, 30 nm to 150 nm, 30 nm to 120 nm, 30 nm to 100 nm, 30 nm to 90 nm, 40 nm to 200 nm, 40 to 180 nm, 40 nm to 170 nm, 40 nm to 150 nm, 40 nm to 120 nm, 40 nm to 100 nm, 40 nm to 90 nm, 40 nm to 80 nm, 40 nm to 70 nm, 50 nm to 200 nm, 50 to 180 nm, 50 nm to 170 nm, 50 nm to 150 nm, 50 nm to 120 nm, 50 nm to 100 nm, 50 nm to 90 nm, 60 nm to 200 nm, 60 to 180 nm, 60 nm to 170 nm, 60 nm to 150 nm, 60 nm to 120 nm, 60 nm to 100 nm, 60 nm to 90 nm, 70 nm to 200 nm, 70 to 180 nm, 70 nm to 170 nm, 70 nm to 150 nm, 70 nm to 120 nm, 70 nm to 100 nm, 70 nm to 90 nm, 80 nm to 200 nm, 80 to 180 nm, 80 nm to 170 nm, 80 nm to 150 nm, 80 nm to 120 nm, 80 nm to 100 nm, 80 nm to 90 nm, 90 nm to 200 nm, 90 to 180 nm, 90 nm to 170 nm, 90 nm to 150 nm, 90 nm to 120 nm, or 90 nm to 100 nm for easy introduction into liver tissue, hepatocytes and/or LSEC (liver sinusoidal endothelial cells). The LNP may be sized for easy introduction into organs or tissues, including but not limited to liver, lung, heart, spleen, as well as to tumors. When the size of the LNP is smaller than the above range, it can be difficult to maintain stability as the surface area of the LNP is excessively increased, and thus delivery to the target tissue and/or drug effect may be reduced. The LNP may specifically target liver tissue. Without wishing to be bound by theory, it is thought that one mechanism by which LNP may be used to deliver therapeutic agents is through the imitation of the metabolic behaviors of natural lipoproteins, and so LNP may be usefully delivered to a subject through the lipid metabolism processes carried out by the liver. During the delivery of therapeutic agents to hepatocytes or and/or LSEC (liver sinusoidal endothelial cells), the diameter of the fenestrae leading from the sinusoidal lumen to the hepatocytes and LSEC is about 140 nm in mammals and about 100 nm in humans, so the LNP composition for therapeutic agent delivery having LNPs with a diameter in the above ranges may have excellent delivery efficiency to hepatocytes and LSEC when compared to LNP having the diameter outside the above range.

According to one example, the LNPs of the LNP composition may comprise the cationic lipid:phospholipid:cholesterol:lipid-PEG conjugate in the range described above or at a molar ratio of 20 to 50:10 to 30:30 to 60:0.5 to 5, at a molar ratio of 25 to 45:10 to 25:40 to 50:0.5 to 3, at a molar ratio of 25 to 45:10 to 20:40 to 55:0.5 to 3, or at a molar ratio of 25 to 45:10 to 20:40 to 55:1.0 to 1.5. The LNP comprising components at a molar ratio in the above range may have excellent delivery efficiency of therapeutic agents specific to cells of target organs.

In certain aspects, the LNP exhibit a positive charge under the acidic pH condition by showing a pKa of about 5 to about 8, about 5.5 to about 7.5, about 6 to about 7, or about 6.5 to about 7, and may encapsulate a nucleic acid with high efficiency by easily forming a complex with a nucleic acid through electrostatic interaction with a therapeutic agent such as a nucleic acid showing a negative charge. In such cases, the LNP may be usefully used as a composition for intracellular or in vivo delivery of a therapeutic agent (for example, nucleic acid).

Herein, “encapsulate” or “encapsulation” refers to incorporation of a therapeutic agent efficient delivery, i.e., by surrounding it by the particle surface and/or embedding it within the particle interior. The encapsulation efficiency means the content of the therapeutic agent encapsulated in the LNP relative the total therapeutic agent content used for preparation of the LNP.

The encapsulation of the nucleic acids of the composition in the LNP may be 70% or more, 75% or more, 80% or more, 85% or more, 90% or more, 91% or more, 92% or more, 94% or more, or 95% or more of LNP in the composition encapsulate nucleic acids. In some embodiments, the encapsulation of the nucleic acids of the composition in the LNP is such that between 80% to 99%, between 80% to 97%, between 80% to 95%, between 85% to 95%, between 87% to 95%, between 90% to 95%, between 91% or more to 95% or less, 91% or more to 94% or less, over 91% to 95% or less, 92% to 99%, between 92% to 97%, or between 92% to 95% of the LNP in the composition encapsulate nucleic acids. In some embodiments, the mRNA encoding the CasX and a gRNA of any of the embodiments of the disclosure are fully encapsulated in the LNP.

The target organs to which a nucleic acid is delivered by the LNP include, but are not limited to the liver, lung, heart, spleen, as well as to tumors. The LNP according to one example is liver tissue-specific and has excellent biocompatibility and can deliver the nucleic acids of a composition with high efficiency, and thus it can be usefully used in related technical fields such as lipid nanoparticle-mediated gene therapy. In a particular embodiment, the target cell to which the nucleic acids are delivered by the LNP according to one example may be a hepatocyte and/or LSEC in vivo. In other embodiments, the disclosure provides LNP formulated for delivery of the nucleic acids of the embodiments to cells ex vivo.

The disclosure provides a pharmaceutical composition comprising a plurality of LNPs comprising nucleic acids, such as mRNA encoding an CasX protein and/or a gRNA variant described herein, and a pharmaceutically acceptable carrier.

In certain embodiments, the LNP comprising the nucleic acid(s) has an electron dense core.

The disclosure provides LNP comprising one or more nucleic acids comprising: (a) an mRNA encoding the CasX, and/or a gRNA variant described herein; (b) one or more cationic lipids or ionizable cationic lipids or salts thereof comprising from about 50 mol % to about 85 mol % of the total lipid present in the LNP; (c) one or more non-cationic lipids comprising from about 13 mol % to about 49.5 mol % of the total lipid present in the LNP; and (d) one or more conjugated lipids that inhibit aggregation of LNPs comprising from about 0.5 mol % to about 2 mol % of the total lipid present in the particle. In another embodiment, the disclosure provides LNP comprising one or more nucleic acids comprising: (a) an mRNA encoding the CasX, and/or a gRNA variant described herein; (b) one or more cationic lipids or ionizable cationic lipids or salts thereof comprising from about 22 mol % to about 85 mol % of the total lipid present in the LNP; (c) one or more non-cationic/phospholipids comprising from about 10 mol % to about 70 mol % of the total lipid present in the LNP; (d) 15 mol % to about 50 mol % sterol, and (d) 1 mol % to about 5 mol % lipid-PEG or lipid-PEG-peptide in the particle. In certain embodiments the CasX mRNA and gRNA may be present in the same nucleic acid-lipid particle, or they may be present in different nucleic acid-lipid particles.

The disclosure provides LNP comprising one or more nucleic acids comprising: (a) an mRNA encoding the CasX described herein; (b) a cationic lipid or a salt thereof comprising from about 52 mol % to about 62 mol % of the total lipid present in the LNP; (c) a mixture of a phospholipid and cholesterol or a derivative thereof comprising from about 36 mol % to about 47 mol % of the total lipid present in the LNP; and (d) a PEG-lipid conjugate comprising from about 1 mol % to about 2 mol % of the total lipid present in the LNP. In particular embodiments, the formulation is a four-component system comprising about 1.4 mol % PEG-lipid conjugate (e.g., PEG2000-C-DMA), about 57.1 mol % cationic lipid (e.g., DLin-K-C2-DMA) or a salt thereof, about 7.1 mol % DPPC (or DSPC), and about 34.3 mol % cholesterol (or derivative thereof).

In other embodiments, the LNP comprising one or more nucleic acids comprises: (a) an mRNA encoding the CasX and/or a gRNA of any of the embodiments described herein; (b) a cationic lipid or a salt thereof comprising from about 46.5 mol % to about 66.5 mol % of the total lipid present in the LNP; (c) cholesterol or a derivative thereof comprising from about 31.5 mol % to about 42.5 mol % of the total lipid present in the LNP; and (d) a PEG-lipid conjugate comprising from about 1 mol % to about 2 mol % of the total lipid present in the LNP. In particular embodiments, the formulation is a three-component system which is phospholipid-free and comprises about 1.5 mol % PEG-lipid conjugate (e.g., PEG2000-C-DMA), about 61.5 mol % cationic lipid (e.g., DLin-K-C2-DMA) or a salt thereof, and about 36.9 mol % cholesterol (or derivative thereof).

Additional formulations are described in PCT Publication No. WO 09/127060 and US patent publication numbers US 2011/0071208 A1 and US 2011/0076335 A1, the disclosures of which are herein incorporated by reference in their entirety.

In other embodiments, the LNP comprising one or more nucleic acids comprises: (a) an mRNA encoding the CasX and a gRNA of any of the embodiments described herein; (b) one or more cationic lipid or ionizable cationic lipids or salts thereof comprising from about 2 mol % to about 50 mol % of the total lipid present in the LNP; (c) one or more non-cationic lipid or ionizable cationic lipids comprising from about 5 mol % to about 90 mol % of the total lipid present in the LNP; and (d) one or more conjugated lipids that inhibit aggregation of particles comprising from about 0.5 mol % to about 20 mol % of the total lipid present in the LNP.

In other embodiments, the LNP comprising one or more nucleic acids comprises: (a) an mRNA encoding the CasX and a gRNA of any of the embodiments described herein; (b) a cationic lipid or a salt thereof comprising from about 30 mol % to about 50 mol % of the total lipid present in the LNP; (c) a mixture of a phospholipid and cholesterol or a derivative thereof comprising from about 47 mol % to about 69 mol % of the total lipid present in the LNP; and (d) a PEG-lipid conjugate comprising from about 1 mol % to about 3 mol % of the total lipid present in the LNP. In particular embodiments, the formulation is a four-component system which comprises about 2 mol % PEG-lipid conjugate (e.g., PEG2000-C-DMA), about 40 mol % cationic lipid (e.g., DLin-K-C2-DMA) or a salt thereof, about 10 mol % DPPC (or DSPC), and about 48 mol % cholesterol (or derivative thereof).

In other embodiments, the LNP comprising one or more nucleic acids comprises: (a) an mRNA encoding the CasX and a gRNA of any of the embodiments described herein; (b) one or more cationic lipid or ionizable cationic lipids or salts thereof comprising from about 50 mol % to about 65 mol % of the total lipid present in the LNP; (c) one or more non-cationic lipid or ionizable cationic lipids comprising from about 25 mol % to about 45 mol % of the total lipid present in the LNP; and (d) one or more conjugated lipids that inhibit aggregation of particles comprising from about 5 mol % to about 10 mol % of the total lipid present in the LNP.

In other embodiments, the LNP comprising one or more nucleic acids comprises: (a) an mRNA encoding the CasX and a gRNA of any of the embodiments described herein; (b) a cationic lipid or a salt thereof comprising from about 50 mol % to about 60 mol % of the total lipid present in the LNP; (c) a mixture of a phospholipid and cholesterol or a derivative thereof comprising from about 35 mol % to about 45 mol % of the total lipid present in the LNP; and (d) a PEG-lipid conjugate comprising from about 5 mol % to about 10 mol % of the total lipid present in the LNP.

In certain embodiments, the non-cationic lipid mixture in the formulation comprises: (i) a phospholipid of from about 10 mol % to about 70 mol % of the total lipid present in the LNP; (ii) cholesterol or a derivative thereof of from about 15 mol % to about 50 mol % of the total lipid present in the LNP; and 1-5% lipid-PEG or lipid-PEG-peptide. In particular embodiments, the formulation is a four-component system which comprises about 7 mol % PEG-lipid conjugate (e.g., PEG750-C-DMA), about 54 mol % cationic lipid (e.g., DLin-K-C2-DMA) or a salt thereof, about 7 mol % DPPC (or DSPC), and about 32 mol % cholesterol (or derivative thereof).

In other embodiments, the LNP comprising one or more nucleic acids comprises: (a) an mRNA encoding the CasX and/or a gRNA of any of the embodiments described herein; (b) a cationic lipid or a salt thereof comprising from about 55 mol % to about 65 mol % of the total lipid present in the LNP; (c) cholesterol or a derivative thereof comprising from about 30 mol % to about 40 mol % of the total lipid present in the LNP; and (d) a PEG-lipid conjugate comprising from about 5 mol % to about 10 mol % of the total lipid present in the LNP. In particular embodiments, the formulation is a three-component system which is phospholipid-free and comprises about 7 mol % PEG-lipid conjugate (e.g., PEG750-C-DMA), about 58 mol % cationic lipid (e.g., DLin-K-C2-DMA) or a salt thereof, and about 35 mol % cholesterol (or derivative thereof).

In other embodiments, the LNP comprising one or more nucleic acids comprises: (a) an mRNA encoding the CasX and/or a gRNA of any of the embodiments described herein; (b) a cationic lipid or a salt thereof comprising from about 48 mol % to about 62 mol % of the total lipid present in the LNP; (c) a mixture of a phospholipid and cholesterol or a derivative thereof, wherein the phospholipid comprises about 7 mol % to about 17 mol % of the total lipid present in the LNP, and wherein the cholesterol or derivative thereof comprises about 25 mol % to about 40 mol % of the total lipid present in the LNP; and (d) a PEG-lipid conjugate comprising from about 0.5 mol % to about 3.0 mol % of the total lipid present in the LNP.

### VII. Systems and Methods for Modification of PCSK9 Target Nucleic Acids

In another aspect, the present disclosure provides systems comprising a CasX nuclease protein and one or more gRNAs for use in modifying or editing a target nucleic acid of a PCSK9 gene in a population of cells. The systems provided herein are useful for various applications, including as therapeutics, diagnostics, and for research. To effect the methods of the disclosure, resulting in modification of the PCSK9 gene, provided herein are programmable CasX:gRNA systems. The programmable nature of the systems provided herein allows for the precise targeting to achieve the desired effect (nicking, cleaving, modifying, etc.) at one or more regions of predetermined interest in the PCSK9 gene target nucleic acid. In some embodiments, it may be desirable to knock-down or knock-out expression of the PCSK9 protein in the subject comprising mutations, for example dominant mutations leading to hypercholesterolemia or familial or autosomal dominant hypercholesterolemia.

A variety of strategies and methods can be employed to modify the PCSK9 target nucleic acid sequence in a cell using the systems provided herein. As used herein “modifying” includes, but is not limited to, cleaving, nicking, editing, deleting, knocking out, knocking down, mutating, correcting, exon-skipping and the like. As described herein, a CasX variant introducing double-stranded cleavage of the target nucleic acid generates a double-stranded break within 18-26 nucleotides 5′ of a PAM site on the target strand and 10-18 nucleotides 3′ on the non-target strand. Depending on the system components utilized, the editing event may be a cleavage event followed by introducing random insertions or deletions (indels) or other mutations (e.g., a substitution, duplication, or inversion of one or more nucleotides), for example by utilizing the imprecise non-homologous DNA end joining (NHEJ) repair pathway, which may generate, for example, a frame shift mutation. In some embodiments of the method, the modification comprises introducing an in-frame mutation in the target nucleic acid. In some embodiments of the method, the modification comprises introducing a frame-shifting mutation in the target nucleic acid. In some embodiments of the method, the modification comprises introducing a premature stop codon in the PCSK9 coding sequence in the target nucleic acid. In some embodiments of the method, the modification results in expression of a non-functional PCSK9 protein in the modified cells of the population. As a result of a gene knock-down, the protein activity or function may be attenuated or the protein levels may be reduced or eliminated. In some embodiments of the method, the modification results in at least about 10%, at least about 20%, at least about 30%, at least about 40%, at least about 50%, at least about 60%, at least about 70%, at least about 80%, or at least about 90% reduced expression of the PCSK9 protein in the modified cells of the population in comparison to cells in which the PCSK9 gene has not been modified.

In some embodiments, the disclosure provides systems specifically designed for use in the methods to modify the target nucleic acid of a PCSK9 gene in eukaryotic cells; either in vitro, ex vivo, or in vivo in a subject. Generally, any portion of the gene can be targeted using the programmable systems and methods provided herein. In one embodiment, the disclosure provides for a method of modifying a target nucleic acid sequence of a PCSK9 gene in a population of cells, the method comprising introducing into each cell of the population: i) a CasX:gRNA system comprising a CasX and a gRNA of any of the embodiments described herein; ii) a nucleic acid encoding the CasX and gRNA of any of the embodiments described herein; iii) a vector selected from the group consisting of a retroviral vector, a lentiviral vector, an adenoviral vector, an adeno-associated viral (AAV) vector, and a herpes simplex virus (HSV) vector, and comprising the nucleic acid of (iv), above; v) an LNP or LNP composition comprising a gRNA and a mRNA encoding the CasX; vi) a synthetic nanoparticle or synthetic nanoparticle composition comprising a gRNA and a mRNA encoding the CasX; or vii) combinations of two or more of (i) to (vi), wherein the target nucleic acid sequence of the cells targeted by the gRNA is modified by the CasX protein. In some embodiments of the method, the PCSK9 target nucleic acid of at least about 1%, at least about 2%, at least about 3%, at least about 4%, at least about 5%, at least about 6%, at least about 7%, at least about 8%, at least about 9%, or at least about 10%, at least about 20%, at least about 30%, at least about 40%, at least about 50%, at least about 60% or more of the cells of the population is modified. In some embodiments of the method, the PCSK9 gene in the cells of the population is modified such that expression of the PCSK9 protein is decreased by at least about 10%, at least about 20%, at least about 30%, at least about 40%, at least about 50%, at least about 60%, at least about 70%, at least about 80%, or at least about 90% in comparison to a cell where the PCSK9 gene has not been modified.

In some embodiments of the method, the modifying of the cell occurs in vitro. In some embodiments of the method, the modifying of the cell occurs ex vivo, wherein the modified cells can be administered to a subject. In some embodiments of the method, the modifying of the cell occurs in vivo. In some embodiments of the method, the cell is a eukaryotic cell. In some embodiments of the method, the eukaryotic cell is selected from the group consisting of a rodent cell, a mouse cell, a rat cell, a primate cell, and a non-human primate cell. In some embodiments of the method, the eukaryotic cell is a human cell. In some embodiments of the method, the cell may be a hepatocyte and/or LSEC (liver sinusoidal endothelial cells). In some embodiments, the cell is selected from the group consisting of hepatocytes, cells of the intestine, cells of the kidney, cells of the central nervous system, smooth muscle cells, macrophages, cells of the retina, and arterial endothelial cells, or combinations thereof. In some embodiments, the eukaryotic cells are selected from the group consisting of embryonic stem cells, induced pluripotent stem cells, germ cells, fibroblasts, oligodendrocytes, glial cells, hematopoietic stem cells, neuron progenitor cells, neurons, astrocytes, muscle cells, bone cells, hepatocytes, pancreatic cells, retinal cells, cancer cells, T-cells, B-cells, NK cells, fetal cardiomyocytes, myofibroblasts, mesenchymal stem cells, autotransplanted expanded cardiomyocytes, adipocytes, totipotent cells, pluripotent cells, blood stem cells, myoblasts, bone marrow cells, mesenchymal cells, parenchymal cells, epithelial cells, an endothelial cells, mesothelial cells, fibroblasts, osteoblasts, chondrocytes, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblast, skeletal myoblast, macrophage, capillary endothelial cells, xenogeneic cells, allogenic cells, or post-natal stem cells.

In some embodiments, the CasX:gRNA systems provided herein for modification of the PCSK9 target nucleic acid comprise an engineered CasX selected from the group consisting of SEQ ID NOS: 4-7, 9-342, 14126-14286, and 14352-14354, or a variant sequence at least 60% identical, at least 70% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical thereto, the gRNA scaffold comprises a sequence selected from the group consisting of SEQ ID NOS: 464-541, or a sequence at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical thereto, and the gRNA comprises a targeting sequence selected from the group consisting of SEQ ID NOS: 544-665 and 2016 or a sequence at least 65% identical, at least 70% identical, at least 75% identical, at least 80% identical, at least 85% identical, at least 90% identical, or at least 95% identical thereto and having between 15 and 30 amino acids. In some embodiments, the CasX:gRNA systems provided herein for modification of the PCSK9 target nucleic acid comprise an engineered CasX selected from the group consisting of SEQ ID NOS: 4-7, 9-342, 14126-14286, and 14352-14354, and the gRNA scaffold comprises a sequence selected from the group consisting of SEQ ID NOS: 464-541. In some embodiments, the CasX:gRNA systems provided herein for modification of the PCSK9 target nucleic acid comprise an engineered CasX selected from the group consisting of SEQ ID NOS: 4-7, 9-342, 14126-14286, and 14352-14354, and the gRNA scaffold comprises a sequence selected from the group consisting of SEQ ID NOS: 465-466 and 513-541. In some embodiments, the CasX:gRNA systems provided herein for modification of the PCSK9 target nucleic acid comprise an engineered CasX selected from the group consisting of SEQ ID NOS: 4-7, 9-342, 14126-14286, and 14352-14354, and the gRNA scaffold comprises a sequence selected from the group consisting of SEQ ID NOS: 464-466. In some embodiments, the CasX:gRNA systems provided herein for modification of the PCSK9 target nucleic acid comprise an engineered CasX selected from the group consisting of SEQ ID NOS: 5-7, 45-342, 14126-14286, and 14352-14354, and the gRNA scaffold comprises a sequence selected from the group consisting of SEQ ID NOS: 464-541. In some embodiments, the CasX:gRNA systems provided herein for modification of the PCSK9 target nucleic acid comprise an engineered CasX selected from the group consisting of SEQ ID NOS: 14126-14286 and 14352-14354, and the gRNA scaffold comprises a sequence selected from the group consisting of SEQ ID NOS: 464-541. In some embodiments, the CasX:gRNA systems provided herein for modification of the PCSK9 target nucleic acid comprise an engineered CasX selected from the group consisting of SEQ ID NOS: 5-7, 45-342, 14126-14286, and 14352-14354, and the gRNA scaffold comprises a sequence selected from the group consisting of SEQ ID NOS: 13749-13757, 13759-13767, 13769-13777 without the spacer represented in the foregoing sequences as undefined nucleotides. In some embodiments, the CasX:gRNA systems provided herein for modification of the PCSK9 target nucleic acid comprise an engineered CasX selected from the group consisting of SEQ ID NOS: 14126-14286 and 14352-14354, and the gRNA scaffold comprises a sequence selected from the group consisting of SEQ ID NOS: 465-466 and 513-541. In some embodiments, the gRNA comprises a targeting sequence selected from the group consisting of SEQ ID NOS: 544-665 and 2016.

In some embodiments of the system, the gRNA comprises a targeting sequence selected from the group consisting of SEQ ID NOS: 544-665 and 2016. In a particular embodiment, the engineered CasX of the system comprises a sequence selected from the group consisting of the sequences of SEQ ID NOS: 5-7, the gRNA scaffold comprises a sequence selected from the group consisting of the sequences of SEQ ID NOS: 464-466, and the targeting sequence of the gRNA of the CasX:gRNA system is selected from the group consisting of the sequence of SEQ ID NOS: 544-559, 583, 619 and 627. In a particular embodiment, wherein the systems are formulated in LNP, the engineered CasX of the sequences selected from the group consisting of SEQ ID NOS: 4-7 are encoded by an mRNA, the gRNA scaffold comprises a sequence of SEQ ID NO: 466, and the targeting sequence of the gRNA of the CasX:gRNA system is selected from the group consisting of the sequence of SEQ ID NOS: 544-559, 583, 619 and 627. In a particular embodiment of the foregoing, the gRNA is chemically modified, including the sequences of 13749-13757, 13759-13767, 13769-13777 with the targeting sequence represented in the foregoing sequences replaced with a targeting sequence selected from the group consisting of the sequence of SEQ ID NOS: 544-559, 583, 619 and 627.

In some embodiments, the systems provided herein for modification of the target nucleic acid comprise an mRNA sequence selected from the group consisting of SEQ ID NOS: 13740-13743, or a sequence at least 60% identical, at least 70% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical thereto. In some embodiments, the systems provided herein for modification of the target nucleic acid comprise an mRNA sequence selected from the group consisting of SEQ ID NOS: 13740-13743. In a particular embodiment, the systems are formulated in LNP that encapsulate the mRNA sequence encoded by a sequence selected from the group consisting of SEQ ID NOS: 13740-13743 and a gRNA selected from the group consisting of SEQ ID NOS: 464-466 and 13748-13777. In other embodiments, the mRNA is encoded by DNA that is incorporated into a vector, such as a recombinant Adeno-Associated Viral (AAV) vector, for delivery of the CasX of the disclosure.

In one embodiment of the method, the system is introduced into the cells using LNP encompassing mRNA encoding the engineered CasX and gRNA variant of any of the embodiments disclosed herein. In some embodiments, the LNP encompasses an mRNA encoding the engineered CasX 515 (SEQ ID NO:5), or a sequence having at least about 70%, at least about 80%, at least about 85%, at least about 90%, at least about 95%, at least about 96%, at least about 97%, at least about 98%, or having at least about 99% sequence identity thereto. In another embodiment, the LNP encompasses an mRNA encoding the engineered CasX 812 (SEQ ID NO: 7), or a sequence having at least about 70%, at least about 80%, at least about 85%, at least about 90%, at least about 95%, at least about 96%, at least about 97%, at least about 98%, or having at least about 99% sequence identity thereto. In another embodiment, the LNP encompasses an mRNA encoding the engineered CasX 491 (SEQ ID NO: 4), or a sequence having at least about 70%, at least about 80%, at least about 85%, at least about 90%, at least about 95%, at least about 96%, at least about 97%, at least about 98%, or having at least about 99% sequence identity thereto. In another embodiment, the LNP encompasses an mRNA encoding the engineered CasX 676 (SEQ ID NO: 6), or a sequence having at least about 70%, at least about 80%, at least about 85%, at least about 90%, at least about 95%, at least about 96%, at least about 97%, at least about 98%, or having at least about 99% sequence identity thereto. In some embodiments of the foregoing, the LNP further encompass a gRNA variant of the disclosure having a targeting sequence complementary to the target nucleic acid. In some embodiments, the LNP comprises gRNA variant 174. In some embodiments, the LNP comprises gRNA variant 235. In some embodiments, the LNP comprises gRNA variant 316. In some embodiments, the LNP comprises gRNA variant 316 with chemical modifications, including the sequences of SEQ ID NOS: 13769-1377. In a particular embodiment, the LNP comprises an mRNA encoding the engineered CasX 515 (SEQ ID NO: 5) and gRNA variant 316 with chemical modifications selected from the group consisting of SEQ ID NOS: 13769-13777, or a sequence having at least about 70%, at least about 80%, at least about 90%, at least about 95%, at least about 96%, at least about 97%, at least about 98%, at least about 99% sequence identity thereto. In some embodiments, the gRNA with chemical modifications comprises a scaffold of SEQ ID NOS: 13769-13777, i.e., a sequence of SEQ ID NOS: 13769-13777 without the spacer represented in the foregoing sequences as undefined nucleotides. In some embodiments of the method, the cells to be modified are selected from the group consisting of rodent cells, mouse cells, rat cells, and non-human primate cells. In other embodiments of the method, the cells to be modified are human cells. In some embodiments of the method, the modification of the population of cells occurs in vivo in a subject, wherein the subject is selected from the group consisting of a rodent, a mouse, a rat, a non-human primate, and a human. In some embodiments of the methods, the modified cell is a hepatocyte, or a cell of the intestine, the kidney, the central nervous system, a smooth muscle cell, macrophage or a cell of arterial walls such as the endothelium.

The LNP can be administered by a route of administration selected from the group consisting of intravenous, intraarterial, intraportal vein injection, intraperitoneal, intramuscular, intracerebroventricular, intracisternal, intrathecal, intracranial, intralumbar, intraocular, subcutaneous, and oral routes.

In other embodiments, the disclosure provides one or more polynucleotides encoding the engineered CasX proteins and gRNAs for use in the methods of modifying the PCSK9 target nucleic acid. In one embodiment of the method, the nucleic acid encoding the CasX:gRNA system can be introduced into the cells by a vector as described herein, or as a plasmid using conventional methods known in the art; e.g. electroporation, microinjection, or chemically. In another embodiment of the method, the system is introduced into the cells using LNP encompassing gRNA and mRNA encoding the engineered CasX. In some embodiments of the method, the cells to be modified are selected from the group consisting of rodent cells, mouse cells, rat cells, and non-human primate cells. In other embodiments of the method, the cells to be modified are human cells. In some embodiments of the method, the modification of the population of cells occurs in vivo in a subject, wherein the subject is selected from the group consisting of a rodent, a mouse, a rat, a non-human primate, and a human. In some embodiments of the methods, the modified cell is a hepatocyte, or a cell of the intestine, the kidney, the central nervous system, a smooth muscle cell, macrophage or a cell of arterial walls such as the endothelium.

In some embodiments of the method of modifying a target nucleic acid sequence, the target nucleic acid sequence comprises a portion of the PCSK9 gene. In some embodiments of the method, the targeting sequence of the gRNA of the system is complementary to a sequence of a PCSK9 exon selected from the group consisting of exon 1, exon 2, exon 3, exon 4, exon 5, exon 6, exon 7, exon 8, exon 9, exon 10, exon 11, and exon 12. In some embodiments of the method, the targeting sequence of the gRNA of the system is complementary to a regulatory element of a PCSK9 gene. In some embodiments of the method, the targeting sequence of the gRNA of the system is complementary to an intergenic sequence of a PCSK9 gene. In some embodiments, the targeting sequence of the gRNA of the system is complementary to donor splice site. In some embodiments, the targeting sequence of the gRNA of the system is complementary to donor splice site, wherein the targeting sequence of the gRNA is complementary to a sequence of a PCSK9 splice-donor site of exon 2. In some embodiments, the targeting sequence of the gRNA of the system is complementary to acceptor splice site. In some embodiments, the targeting sequence of the gRNA of the system is complementary to acceptor splice site, wherein the targeting sequence of the gRNA is complementary to a sequence of a PCSK9 splice-acceptor site of exon 2, exon 5, exon 6, or exon 11.

In some embodiments of the method, vectors may be provided directly to a target host cell. For example, cells may be contacted with vectors having nucleic acids encoding the CasX and gRNA of any of the embodiments described herein such that the vectors are taken up by the cells. For viral vector delivery, cells can be contacted with viral particles comprising the subject viral expression vectors and the nucleic acid encoding the CasX and gRNA. In some embodiments, the vector is an Adeno-Associated Viral (AAV) vector, wherein the AAV is selected from AAV1, AAV2, AAV3, AAV4, AAV5, AAV6, AAV7, AAV8, AAV9, AAV10, AAV11, AAV12, AAV 9.45, AAV 9.61, AAV-Rh74, or AAVRh10.

The vector can be administered by a route of administration selected from the group consisting of intravenous, intraportal vein injection, intraperitoneal, intramuscular, subcutaneous, and oral routes. The LNP can be administered by intravenous or intraportal vein injection.

The systems and methods described herein can be used in a variety of cells associated with disease, e.g., cells of the liver, the intestine, the kidney, the central nervous system, smooth muscle cells, macrophages or cells of arterial walls, in which the PCSK9 gene is modified or knocked out. This approach, therefore, could be used to modify cells for applications in a subject with a PCSK9-related disorder such as, but not limited to autosomal dominant hypercholesterolemia (ADH), hypercholesterolemia, elevated total cholesterol levels, hyperlipidemia, elevated low-density lipoprotein (LDL) levels, elevated LDL-cholesterol levels, reduced high-density lipoprotein levels, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, age-related macular degeneration (AMD), or a combination thereof.

### VIII. Therapeutic Methods

The present disclosure provides methods of treating a PCSK9-related disorder in a subject in need thereof, including, but not limited to, autosomal dominant hypercholesterolemia (ADH), hypercholesterolemia, elevated total cholesterol levels, elevated low-density lipoprotein (LDL) levels, reduced high-density lipoprotein levels, liver steatosis, atherosclerotic cardiovascular disease, and coronary artery disease, ischemia, stroke, peripheral vascular disease, thrombosis, type 2 diabetes, high elevated blood pressure, obesity, Alzheimer's disease, neurodegeneration, age-related macular degeneration (AMD), or a combination thereof. In some embodiments, the methods of the disclosure can prevent, treat and/or ameliorate a PCSK9-related disorder of a subject by the administering to the subject of a composition of the disclosure. In some embodiments, the composition administered to the subject further comprises pharmaceutically acceptable carrier, diluent or excipient.

In some cases, one or both alleles of the PCSK9 gene of the subject comprises a mutation. In some cases, the PCSK9-related disorder mutation is a gain of function mutation, including, but not limited to, mutations encoding amino acid substitutions selected from the group consisting of S127R, D129G, F216L, D374H, and D374Y relative to the sequence of SEQ ID NO:543.

In some embodiments, the disclosure provides methods of treating a PCSK9 or related disorder in a subject in need thereof comprising modifying a PCSK9 gene in a cell of the subject, the modifying comprising contacting said cells with a therapeutically effective dose of: i) a CasX:gRNA system comprising a CasX and a gRNA of any one of the embodiments described herein; ii) a nucleic acid encoding the CasX and gRNA of any of the embodiments described herein; iii) a vector selected from the group consisting of a retroviral vector, a lentiviral vector, an adenoviral vector, an adeno-associated viral (AAV) vector, and a herpes simplex virus (HSV) vector, and comprising the nucleic acid of (iv), above; v) an LNP or LNP composition comprising a gRNA and a mRNA encoding the CasX; vi) a synthetic nanoparticle or synthetic nanoparticle composition comprising a gRNA and a mRNA encoding the CasX; or vi) combinations of two or more of (i) to (vi), wherein the target nucleic acid sequence of the cells targeted by the gRNA is modified by the CasX protein. In some embodiments of the method, the PCSK9 target nucleic acid of at least about 1%, at least about 2%, at least about 3%, at least about 4%, at least about 5%, at least about 6%, at least about 7%, at least about 8%, at least about 9%, or at least about 10%, at least about 20%, at least about 30%, at least about 40%, at least about 50%, at least about 60% or more of the cells of the population is modified. In some embodiments of the method, the PCSK9 gene in the cells of the population is modified such that expression of the apo (a) protein is decreased by at least about 10%, at least about 20%, at least about 30%, at least about 40%, at least about 50%, at least about 60%, at least about 70%, at least about 80%, or at least about 90% in comparison to a cell where the PCSK9 gene has not been modified. The modified cell of the treated subject can be a eukaryotic cell selected from the group consisting of a rodent cell, a mouse cell, a rat cell, a primate cell, a non-human primate cell, and a human cell. In some embodiments, the eukaryotic cell of the treated subject is a human cell. In some embodiments, the cell is a cell involved in the production of LDL, including but not limited to a hepatocyte, or a cell of the intestine, the kidney, the central nervous system, a smooth muscle cell, macrophage, a retinal cell, or cell of arterial walls such as the endothelium. In some embodiments, the cell is an eye cell.

In one particular embodiment of a method of treating a PCSK9 or related disorder in a subject in need thereof, the vector for use in the method is an AAV having a serotype selected from the group consisting of AAV1, AAV2, AAV3, AAV4, AAV5, AAV6, AAV7, AAV8, AAV9, AAV10, AAV11, AAV12, AAV 9.45, AAV 9.61, AAV-Rh74, or AAVRh10. In some embodiments of the method, the AAV vector of the embodiments is administered to the subject at a therapeutically effective dose.

The vector, LNP, or pharmaceutical composition of the embodiments can be administered by a route of administration selected from the group consisting of intravenous, intraportal vein injection, intraperitoneal, intramuscular, subcutaneous, intraocular, and oral routes. In some embodiments of the methods of treating a PCSK9-related disorder in a subject, the subject is selected from the group consisting of mouse, rat, pig, non-human primate, and human.

In some embodiments, the method of treating a PCSK9-related disease or disorder in a subject comprises pretreating the subject with a therapeutic agent that increases hepatic LDL receptor (LDLR) expression. In some embodiments, the therapeutic agent is a PCSK9 inhibitor, such as a monoclonal antibody, nucleic acid-based agent, or a small molecule. Exemplary therapeutic agents include, but are not limited to, evolocumab, inclisiran, alirocumab, and MK-0616. Without wishing to be bound by theory or mechanism, it is believed that the pretreatment with an inhibitor of PCSK9 may lead to an increase in hepatic LDL receptor (LDLR) expression that, in turn, may facilitate the uptake of the LNP comprising the CasX:gRNA composition that is subsequently administered to the subject. By increasing the hepatic cell uptake of the LNP, it is expected that editing of the PCSK9 gene will be enhanced such that an improvement in the PCSK9-related disorder would be attained.

A number of therapeutic strategies have been used to design the systems for use in the methods of treatment of a subject with a PCSK9-related disorder. In some embodiments, the disclosure provides a method of treatment of a subject having a PCSK9-related disorder, the method comprising administering to the subject a CasX:gRNA composition or a vector of any of the embodiments disclosed herein (e.g., an AAV or an LNP) according to a treatment regimen comprising one or more consecutive doses using a therapeutically effective dose. In some embodiments of the treatment regimen, the therapeutically effective dose of the composition or vector is administered as a single dose. In other embodiments of the treatment regimen, the therapeutically effective dose is administered to the subject as two or more doses over a period of at least two weeks, or at least one month, or at least two months, or at least three months, or at least four months, or at least five months, or at least six months. In some embodiments of the treatment regiment, the effective doses are administered by a route selected from the group consisting of intravenous, intraportal vein injection, intraperitoneal, intramuscular, subcutaneous, intraocular, and oral routes.

In some embodiments, the administering a therapeutically effective amount of a CasX:gRNA modality, including a vector or LNP comprising a polynucleotide encoding a CasX protein and a guide ribonucleic acid disclosed herein, to knock down or knock out expression of PCSK9 in a subject with a PCSK9-related disorder, leads to the prevention or amelioration of the underlying PCSK9-related disorder such that an improvement is observed in the subject, notwithstanding that the subject may still be afflicted with the underlying disorder. In some embodiments, administration of the therapeutically effective amount of the CasX:gRNA modality leads to an improvement in at least one clinically-relevant endpoint including, but not limited to, percent change from baseline in LDL-cholesterol, decrease in plaque atheroma volume, reduction in in coronary plaque, reduction in atherosclerotic cardiovascular disease (ASCVD), cardiovascular death, nonfatal myocardial infarction, ischemic stroke, nonfatal stroke, coronary revascularization, unstable angina, or visual acuity. In some embodiments, administration of the therapeutically effective amount of the CasX-gRNA modality leads to an improvement in at least two clinically-relevant endpoints. In some embodiments, the subject is selected from mouse, rat, pig, dog, non-human primate, and human. In some embodiments, the subject is human.

In some embodiments, the methods of treatment further comprise administering a chemotherapeutic agent effective in lowering LDL levels. Such agents include, but are not limited to, statins, niacin, fibrates, or anti-PCSK9 antibody drugs.

Methods of obtaining samples from treated subjects for analysis to determine the effectiveness of the treatment, such as body fluids or tissues, and methods of preparation of the samples to allow for analysis, are well known to those skilled in the art. Methods for analysis of RNA and protein levels are discussed above and are well known to those skilled in the art. The effects of treatment can also be assessed by measuring biomarkers associated with the target gene expression in the aforementioned fluids, tissues or organs, collected from an animal contacted with one or more compounds of the disclosure, by routine clinical methods known in the art. Biomarkers of PCSK9 disorders include, but are not limited to, PCSK9 levels, low-density lipoprotein (LDL-cholesterol), apolipoprotein B, non-HDL cholesterol, triglycerides and lipoprotein a, soluble CD40 ligand, osteopontin (OPN), osteoprotegerin (OPG), matrix metalloproteinases (MMP) and myeloperoxidase (MPOP), wherein the concentration of the marker is compared to concentrations known to be physiologically normal or in subjects not having a PCSK9 disorder.

Several mouse models expressing mutant forms of PCSK9 exist and are suitable for evaluating the methods of treatment. Transgenic mouse models of PCSK9-related disorders include knock-in mouse models having hPCSK9 (Carreras, A. In vivo genome and base editing of a human PCSK9 knock-in hypercholesterolemic mouse model. MC Biology 17:4 (2019); Herbert B., et al. Increased secretion of lipoproteins in transgenic mice expressing human D374Y PCSK9 under physiological genetic control. Arterioscler Thromb Vasc Biol. 30(7): 1333 (2010)).

### IX. Pharmaceutical Compositions, Kits, and Articles of Manufacture

The disclosure provides pharmaceutical compositions comprising: i) a CasX protein and a gRNA comprising a targeting sequence specific for a PCSK9 gene; ii) one or more nucleic acids encoding the CasX and the gRNA of (i); iii) a vector comprising the one or more nucleic acids of (ii); iv) an LNP or LNP composition comprising a gRNA and a mRNA encoding the CasX; or v) a synthetic nanoparticle or synthetic nanoparticle composition comprising a gRNA and a mRNA encoding the CasX, together with one or more pharmaceutically suitable excipients. In some embodiments, the pharmaceutical composition is formulated for a route of administration selected from the group consisting of intravenous, intraportal vein injection, intraperitoneal, intramuscular, subcutaneous, intraocular, and oral routes. In some embodiments, the pharmaceutical composition is in a liquid form or a frozen form. In other embodiments, the pharmaceutical composition is in a pre-filled syringe for a single injection. In other embodiments, the pharmaceutical composition is in solid form, for example the pharmaceutical composition is lyophilized.

In other embodiments, provided herein are kits comprising a CasX protein and a CasX gRNA comprising a targeting sequence specific for a PCSK9 gene and a suitable container (for example a tube, vial or plate). In exemplary embodiments, the kit comprises an engineered CasX selected from the group consisting of SEQ ID NOS: 4-7, 9-342, 14126-14286, and 14352-14354. In further embodiments, the kit comprises an engineered CasX selected from the group consisting of SEQ ID NOS: 45-342, 14126-14286, and 14352-14354. In still further embodiments, the kit comprises an engineered CasX selected from the group consisting of SEQ ID NOS: 14126-14286 and 14352-14354.

In some embodiments, the kit comprises a gRNA or a vector encoding a gRNA, wherein the gRNA scaffold comprises a sequence selected from the group consisting of SEQ ID NOS: 464-541. In some embodiments, the kit comprises a gRNA or a vector encoding a gRNA, wherein the gRNA scaffold comprises a sequence selected from the group consisting of SEQ ID NOS: 465-466 and 513-541. In some embodiments, the gRNA comprises a sequence selected from the group consisting of SEQ ID NOS: 464-466, or a chemically modified version thereof.

In certain embodiments, provided herein are kits comprising a CasX protein and gRNA editing pair. In some embodiments, the editing pair comprises an engineered CasX protein selected from the group consisting of SEQ ID NOS: 4-7, 9-342, 14126-14286, and 14352-14354, and a gRNA variant comprising a scaffold of any one of SEQ ID NOS: 465-466 and 513-541. In some embodiments, the editing pair comprises an engineered CasX protein selected from the group consisting of SEQ ID NOS: 5-7, 9-342, 14126-14286, and 14352-14354, and a gRNA variant comprising a scaffold of any one of SEQ ID NOS: 464-466. In some embodiments, the editing pair comprises an engineered CasX protein selected from the group consisting of SEQ ID NOS: 14126-14286 and 14352-14354, and a gRNA variant comprising a scaffold of any one of SEQ ID NOS: 464-466. In some embodiments, the gRNA of the gene editing pair comprises a targeting sequence of any one of SEQ ID NOS: 544-559, 583, 619 and 627.

In some embodiments, the kit further comprises a buffer, a nuclease inhibitor, a protease inhibitor, a liposome, a therapeutic agent, a label, a label visualization reagent, or any combination of the foregoing. In some embodiments, the kit further comprises a pharmaceutically acceptable carrier, diluent or excipient. In some embodiments, the kit further comprises instructions for use.

In some embodiments, the kit comprises appropriate control compositions for gene modifying applications, and instructions for use.

In some embodiments, the kit comprises a vector comprising a sequence encoding a CasX protein of the disclosure and a CasX gRNA of the disclosure.

The following Examples are merely illustrative and are not meant to limit any aspects of the present disclosure in any way.

### Examples

### Example 1: CasX Molecule 119 and Guide Scaffold 174 Edits PCSK9 Locus in HEK293T Cells

The purpose of the experiments was to demonstrate editing of the PCSK9 locus in HEK293T cells using constructs of CasX 119, guide 174 and spacers targeting the WT sequence, when delivered by plasmid transfection.

### Materials and Methods

Spacers targeting PCSK9 were chosen manually based on PAM availability without prior knowledge of activity (sequences in Table 12). HEK293T cells were seeded at 20-40k cells/well in a 96 well plate in 100 μl of fibroblast (FB) medium and cultured in a 37° C. incubator with 5% CO2. The following day, confluence of seeded cells was checked to ensure that cells were at ~75% confluence at time of transfection. If cells were at the right confluence, transfection was carried out. Each CasX and guide construct (e.g., see Table 11 for sequence of guide 174; see Table 12 for PCSK9 spacer sequences; CasX119 is SEQ ID NO: 8) was transfected into the HEK293T cells at 100-500 ng per well using Lipofectamine 3000 following the manufacturer's protocol, using 3 wells per construct as replicates. SaCas9 and SpyCas9 targeting PCSK9 were used as benchmarking controls. For each Cas protein type, a non-targeting plasmid was used as a negative control. Cells were selected for successful transfection with puromycin at 0.3-3 μg/ml for 24-48 hours, followed by 24-96 hours of recovery in FB medium. Cells for each sample from the experiment was lysed, and the genome was extracted following the manufacturer's protocol and standard practices. Editing in cells from each experimental sample were assayed using NGS analysis. Briefly, genomic DNA was amplified via PCR with primers specific to the target genomic location of interest to form a target amplicon. These primers contained an additional sequence at the 5′ ends to introduce Illumina reads 1 and 2 sequences. Further, they contained a 16-nucleotide random sequence that functioned as a unique molecular identifier (UMI). The quality and quantification of the amplicon was assessed using a Fragment Analyzer DNA analyzer kit (Agilent, dsDNA 35-1500 bp). Amplicons were sequenced on the Illumina MiSeq™ according to the manufacturer's instructions. Raw fastq sequencing files were processed by trimming for quality and adapter sequences and merging read 1 and read 2 into a single insert sequence; insert sequences were then analyzed by the CRISPResso2 (v 2.0.29) program. The percentage of reads modified in a window around the 3′ end of the spacer was determined. The activity of the CasX molecule was quantified as the total percent of reads that contain insertions and/or deletions anywhere within this window.

TABLE 12
Spacer sequences targeting PCSK9 locus.

| | | | DNA SEQ | RNA SEQ |
|---|---|---|---|---|
| Gene | Spacer | PAM | ID NO: | ID NO: |
| PCSK9 | 6.1 | TTC | 13782 | 619 |
| PCSK9 | 6.2 | TTC | 13783 | 621 |
| PCSK9 | 6.4 | TTC | 13784 | 618 |
| PCSK9 | 6.5 | TTC | 13785 | 620 |
| PCSK9 | 6.3 | TTC | 13786 | 645 |
| PCSK9 | 6.6 | TTC | 13787 | 551 |
| PCSK9 | 6.7 | TTC | 13788 | 552 |
| PCSK9 | 6.8 | TTC | 13789 | 569 |
| PCSK9 | 6.9 | TTC | 646 | 646 |
| PCSK9 | 6.10 | TTC | 13790 | 550 |

### Results

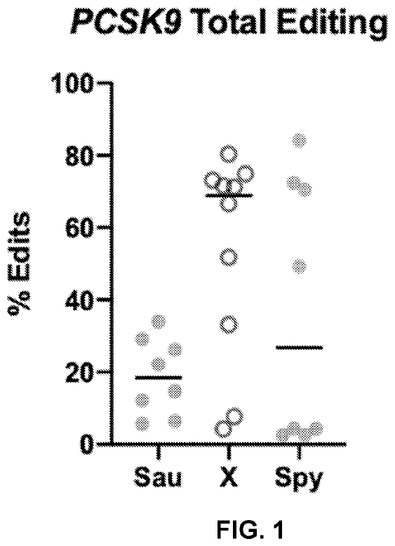

The graph of FIG. 1 shows that constructs utilizing ten different spacers targeted to PCSK9 were able to edit the PCSK9 locus with varying levels of activity, at an average editing of 70%. Each data point is an average measurement of NGS reads of editing outcomes generated by an individual spacer. These results demonstrate that, under the conditions of the assay, CasX with appropriate guides were able to edit the PCSK9 locus and did so to a greater degree compared to Spy Cas9 (based on mean editing), while exhibiting considerably more editing than Sau Cas9.

### Example 2: CasX 119 and Guide Scaffold 174 Edits the PCSK9 Locus in HepG2 Cells

Experiments were conducted to demonstrate the ability to edit the PCSK9 locus in HepG2 cells using constructs of CasX 119, guide 174 and spacers targeting the WT PCSK9 sequence delivered by lentivirus.

### Materials and Methods

Lentiviral particles were produced using standard methods by transfecting HEK293T at a confluency of 70%-90% using polyethylenimine-based transfection of CasX plasmids containing spacers targeting the PCSK9 locus (sequences 6.7, 6.8, and 6.9 of Table 12), the lentiviral packaging plasmid and the VSV-G envelope plasmids. For particle production, media was changed 12 hours post-transfection, and virus harvested at 36-48 hours post-transfection. Viral supernatants were filtered using 0.45 μm membrane filters, diluted in media if appropriate, and added to HepG2 target cells cultured in HepG2 medium (EMEM with 10% FBS and 1% penicillin-streptomycin). Supplemental polybrene was added at 5-20 μg/ml to enhance transduction efficiency, if necessary. Transduced cells were selected 24-48 hours post-transduction using puromycin at 0.3-3 μg/ml in HepG2 medium and grown for 6 days in HepG2 medium in a 37° C. incubator with 5% CO2. Cells were then harvested, and editing was analyzed using NGS. Briefly, genomic DNA was amplified via PCR with primers specific to the target genomic location of interest to form a target amplicon. These primers contained an additional sequence at the 5′ ends to introduce Illumina reads 1 and 2 sequences. Further, they contained a 16-nucleotide random sequence that functioned as a unique molecular identifier (UMI). The quality and quantification of the amplicon was assessed using a Fragment Analyzer DNA analyzer kit (Agilent, dsDNA 35-1500 bp). Amplicons were sequenced on the Illumina MiSeq™ according to the manufacturer's instructions. Raw fastq sequencing files were processed by trimming for quality and adapter sequences and merging read 1 and read 2 into a single insert sequence; insert sequences were then analyzed by the CRISPResso2 (v 2.0.29) program. The percentage of reads modified in a window around the 3′ end of the spacer was determined. The activity of the CasX molecule was quantified as the total percent of reads that contain insertions and/or deletions anywhere within this window.

### Results

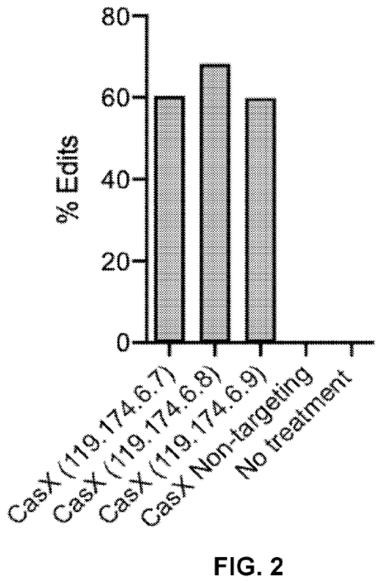

The graph of FIG. 2 shows that constructs with three different spacers targeted to PCSK9 were able to edit the PCSK9 locus with varying levels of activity, at an average editing of 60%. Each data point is an average measurement of NGS reads of editing outcomes generated by an individual spacer.

The results demonstrate that, under the conditions of the assay, CasX with appropriately targeted guides were able to edit the PCSK9 locus in HepG2 cells with a high degree of efficiency.

### Example 3: CasX 491 and Guide Scaffold 174 Edits the PCSK9 Locus in AML12 Cells

Experiments were conducted to demonstrate the ability to edit the wild-type PCSK9 locus in AML12 cells when delivered by transfection.

### Materials and Methods

Murine hepatocyte cell line AML12 cells were transfected with 1000 ng of plasmid encoding CasX 491 along with gRNA scaffold 174 with spacers 27.1 to 27.7, targeting wild-type murine PCSK9 (sequence in Table 13). Transfected cells were grown for 6 days in AML12 medium (DMEM:F12 supplemented with 10% fetal bovine serum, 10 μg/ml insulin, 5.5 μg/ml transferrin, 5 ng/ml selenium, 40 ng/ml dexamethasone) incubated at 37° C. incubator with 5% CO2. Cells were then harvested, and editing was analyzed using NGS. Briefly, genomic DNA was amplified via PCR with primers specific to the target genomic location of interest to form a target amplicon. These primers contained an additional sequence at the 5′ ends to introduce Illumina reads 1 and 2 sequences. Further, they contained a 16-nucleotide random sequence that functioned as a unique molecular identifier (UMI). The quality and quantification of the amplicon was assessed using a Fragment Analyzer DNA analyzer kit (Agilent, dsDNA 35-1500 bp). Amplicons were sequenced on the Illumina MiSeq™ according to the manufacturer's instructions. Raw fastq sequencing files were processed by trimming for quality and adapter sequences and merging read 1 and read 2 into a single insert sequence; insert sequences were then analyzed by the CRISPResso2 (v 2.0.29) program. The percentage of reads modified in a window around the 3′ end of the spacer was determined. The activity of the CasX molecule was quantified as the total percent of reads that contain insertions and/or deletions anywhere within this window.

TABLE 13
Spacer sequences targeting mouse PCSK9 genetic locus

| | | Spacer DNA | DNA SEQ | Spacer RNA | RNA SEQ ID |
|---|---|---|---|---|---|
| Spacer | PAM | Sequence | ID NO: | Sequence | NO: |
| 27.1 | TTC | GCCTCGCCCTCCCCAGACAG | 13791 | GCCUCGCCCUCCCCAGACAG | 13798 |
| 27.2 | TTC | GATGGGGCTCGGGGTGGCGT | 13792 | GAUGGGGCUCGGGGUGGCGU | 13799 |
| 27.3 | TTC | GGGGTGTGGGTACTGGACGC | 13793 | GGGGUGUGGGUACUGGACGC | 13800 |
| 27.4 | TTC | CGTGGACGCGCAGGCTGCCG | 13794 | CGUGGACGCGCAGGCUGCCG | 13801 |
| 27.5 | TTC | GAGGCTAGAGGACTGAGCCA | 13795 | GAGGCUAGAGGACUGAGCCA | 13802 |
| 27.6 | TTC | CGAGGCCGCGCGCACCTCTC | 13796 | CGAGGCCGCGCGCACCUCUC | 13803 |
| 27.7 | TTC | TAATCTCCATCCTCGTCCTG | 13797 | UAAUCUCCAUCCUCGUCCUG | 13804 |

### Results

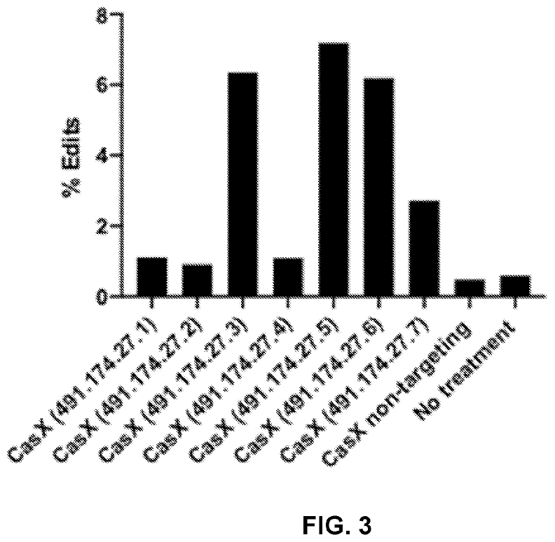

The graph of FIG. 3 shows that constructs with three different spacers were able to edit the PCSK9 locus with an average editing of at least 6-7%, with other spacers resulting in lower amounts of editing. Each data point is an average measurement of NGS reads of editing outcomes generated by an individual spacer. The results demonstrate that, under the conditions of the assay, CasX with appropriately targeted guides were able to edit the PCSK9 locus in AML12 cells.

Example 4: Use of CasX:gRNA Systems to Edit the Human PCSK9 Locus In Vitro

Experiments were performed to demonstrate that small CRISPR proteins, such as CasX, and guide RNAs (gRNA), can edit the human PCSK9 locus in HEK293T cells.

### Materials and Methods

### Lentiviral Plasmid Cloning:

Lentiviral plasmid constructs comprising sequences coding for CasX protein 491, guide scaffold variant 235, and PCSK9-targeting spacers (Table 14) were generated and cloned upstream of a P2A-mScarlet coding region on a lentiviral plasmid using standard molecular cloning techniques. Spacers were chosen manually based on availability of TTC or ATC PAM availability throughout the human PCSK9 locus. Cloned and sequence-validated constructs were midi-prepped and subjected to quality assessment prior to transfection into HEK293T cells.

TABLE 14
Sequences of spacers targeting the human PCSK9 locus (bolded
spacers were screened and assessed in this example).

| Spacer ID | PAM | DNA SEQ ID NO: | RNA SEQ ID NO: |
|---|---|---|---|
| 6.1* | TTC | 13782 | 619 |
| 6.2* | TTC | 13783 | 621 |
| 6.3 | TTC | 13786 | 645 |
| 6.4 | TTC | 13784 | 618 |
| 6.5 | TTC | 13785 | 620 |
| 6.6 | TTC | 13787 | 551 |
| 6.7* | TTC | 13788 | 552 |
| 6.8* | TTC | 13789 | 569 |
| 6.9* | TTC | 646 | 646 |
| 6.10 | TTC | 13790 | 550 |
| 6.59 | ATC | 13805 | 5463 |
| 6.74 | ATC | 13806 | 5538 |
| 6.75 | ATC | 13807 | 5539 |
| 6.79 | ATC | 13808 | 5541 |
| 6.86 | TTC | 13809 | 570 |
| 6.87 | TTC | 13810 | 544 |
| 6.88 | TTC | 13811 | 647 |
| 6.93 | TTC | 13812 | 648 |
| 6.96 | ATC | 13813 | 5588 |
| 6.98 | ATC | 13814 | 5590 |
| 6.102 | ATC | 13815 | 5591 |
| 6.103 | TTC | 13816 | 649 |
| 6.105 | TTC | 13817 | 2404 |
| 6.106 | TTC | 13818 | 650 |
| 6.107 | TTC | 13819 | 651 |
| 6.108 | TTC | 13820 | 652 |
| 6.109* | TTC | 13821 | 644 |
| 6.110 | TTC | 13822 | 554 |
| 6.111 | TTC | 13823 | 653 |
| 6.112* | TTC | 13824 | 654 |
| 6.114* | TTC | 13825 | 553 |
| 6.115* | TTC | 13826 | 655 |
| 6.116* | TTC | 13827 | 656 |
| 6.118 | TTC | 13828 | 622 |
| 6.119 | TTC | 13829 | 623 |
| 6.120 | TTC | 13830 | 624 |
| 6.121 | TTC | 13831 | 627 |
| 6.122 | TTC | 13832 | 625 |
| 6.123 | TTC | 13833 | 626 |
| 6.124 | TTC | 13834 | 628 |
| 6.125 | TTC | 13835 | 629 |
| 6.126 | TTC | 13836 | 630 |
| 6.127 | TTC | 13837 | 631 |
| 6.149 | TTC | 13838 | 632 |
| 6.150 | TTC | 13839 | 633 |
| 6.151 | TTC | 13840 | 634 |
| 6.154 | TTC | 13841 | 559 |
| 6.155 | TTC | 13842 | 560 |
| 6.156 | TTC | 13843 | 561 |
| 6.157 | TTC | 13844 | 562 |
| 6.158 | TTC | 13845 | 563 |
| 6.160 | TTC | 13846 | 564 |
| 6.161 | TTC | 13847 | 565 |
| 6.162 | ATC | 13848 | 5711 |
| 6.163 | ATC | 13849 | 5714 |
| 6.164 | ATC | 13850 | 5542 |
| 6.188 | TTC | 13851 | 566 |
| 6.189 | TTC | 13852 | 567 |
| 6.190 | TTC | 13853 | 568 |
| 6.191 | TTC | 13854 | 571 |
| 6.192 | TTC | 13855 | 572 |
| 6.193 | TTC | 13856 | 573 |
| 6.194 | TTC | 13857 | 574 |
| 6.195 | TTC | 13858 | 575 |
| 6.196 | TTC | 13859 | 576 |
| 6.197 | TTC | 13860 | 577 |
| 6.198 | TTC | 13861 | 578 |
| 6.199 | TTC | 13862 | 579 |
| 6.200 | TTC | 13863 | 580 |
| 6.201 | TTC | 13864 | 581 |
| 6.202 | TTC | 13865 | 582 |
| 6.203 | TTC | 13866 | 583 |
| 6.204 | TTC | 13867 | 584 |
| 6.205 | TTC | 13868 | 585 |
| 6.206 | TTC | 13869 | 586 |
| 6.207 | TTC | 13870 | 587 |
| 6.208 | TTC | 13871 | 588 |
| 6.209 | TTC | 13872 | 589 |
| 6.210 | TTC | 13873 | 545 |
| 6.211 | TTC | 13874 | 590 |
| 6.212 | TTC | 13875 | 591 |
| 6.213 | TTC | 13876 | 546 |
| 6.214 | TTC | 13877 | 547 |
| 6.215 | TTC | 13878 | 548 |
| 6.216 | TTC | 13879 | 549 |
| 6.217 | TTC | 13880 | 592 |
| 6.218 | TTC | 13881 | 593 |
| 6.219 | TTC | 13882 | 594 |
| 6.220 | TTC | 13883 | 595 |
| 6.221 | TTC | 13884 | 596 |
| 6.222 | TTC | 13885 | 597 |
| 6.223 | TTC | 13886 | 598 |
| 6.224 | TTC | 13887 | 599 |
| 6.225 | TTC | 13888 | 600 |
| 6.226 | TTC | 13889 | 60 |
| 6.227 | TTC | 13890 | 602 |
| 6.228 | TTC | 13891 | 603 |
| 6.229 | TTC | 13892 | 604 |
| 6.230 | TTC | 13893 | 605 |
| 6.231 | TTC | 13894 | 606 |
| 6.232 | TTC | 13895 | 607 |
| 6.233 | TTC | 13896 | 608 |
| 6.234 | TTC | 13897 | 609 |
| 6.235 | TTC | 13898 | 610 |
| 6.236 | TTC | 13899 | 611 |
| 6.237 | TTC | 13900 | 612 |
| 6.238 | TTC | 13901 | 613 |
| 6.239 | TTC | 13902 | 614 |
| 6.240 | TTC | 13903 | 615 |
| 6.241 | TTC | 13904 | 616 |
| 6.242 | TTC | 13905 | 617 |
| 6.243 | TTC | 13906 | 635 |
| 6.244 | TTC | 13907 | 636 |
| 6.245 | TTC | 13908 | 637 |
| 6.246 | TTC | 13909 | 638 |
| 6.247 | TTC | 13910 | 639 |
| 6.248 | TTC | 13911 | 640 |
| 6.249 | TTC | 13912 | 641 |
| 6.250 | TTC | 13913 | 642 |
| 6.251 | TTC | 13914 | 643 |
| 6.252 | ATC | 13915 | 5455 |
| 6.253 | ATC | 13916 | 5458 |
| 6.254 | ATC | 13917 | 5461 |
| 6.255 | ATC | 13918 | 5587 |
| 6.256 | ATC | 13919 | 5593 |
| 6.257 | ATC | 13920 | 5594 |
| 6.258 | ATC | 13921 | 5712 |
| 6.259 | ATC | 13922 | 5713 |
| 6.260 | ATC | 13923 | 5717 |
| 6.261 | ATC | 13924 | 5718 |
| 6.262 | ATC | 13925 | 5720 |
| 6.263 | ATC | 13926 | 5721 |
| 6.264 | ATC | 13927 | 5776 |
| 6.265 | ATC | 13928 | 5777 |
| 6.266 | ATC | 13929 | 5793 |
| 6.267 | ATC | 13930 | 5794 |
| 6.268 | ATC | 13931 | 5795 |
| 6.269 | ATC | 13932 | 5796 |
| 6.270 | ATC | 13933 | 5806 |
| 6.271 | ATC | 13934 | 5807 |
| 6.272 | ATC | 13935 | 5808 |
| 6.273 | ATC | 13936 | 5809 |
| 6.274 | ATC | 13937 | 5811 |
| 6.275 | ATC | 13938 | 5810 |
| 6.276 | ATC | 13939 | 5812 |
| 6.277 | ATC | 13940 | 5813 |
| 6.278 | ATC | 13941 | 5817 |
| 6.279 | ATC | 13942 | 5818 |
| 6.280 | ATC | 5819 | 5819 |
| 6.282 | ATC | 13943 | 5840 |
| 6.283 | ATC | 13944 | 5841 |
| 6.284 | ATC | 13945 | 5870 |
| 6.285 | ATC | 13946 | 5871 |
| 6.286 | ATC | 13947 | 5872 |
| 6.287 | ATC | 13948 | 5873 |
| 6.288 | ATC | 13949 | 5912 |
| 6.289 | ATC | 13950 | 5911 |
| 6.290 | ATC | 13951 | 5913 |
| 6.291 | ATC | 13952 | 5914 |
| 6.293 | ATC | 13953 | 5916 |
| 6.307 | ATC | 13954 | 5932 |
| 6.310 | ATC | 13955 | 5935 |
| 6.311 | ATC | 13956 | 5432 |
| 6.313 | ATC | 13957 | 5433 |
| 6.314 | ATC | 13958 | 5434 |
| 6.315 | ATC | 13959 | 5437 |
| 6.316 | ATC | 13960 | 5438 |
| 6.317 | ATC | 13961 | 5439 |
| 6.318 | ATC | 13962 | 5440 |
| 6.319 | ATC | 13963 | 5450 |
| 6.320 | ATC | 13964 | 5451 |
| 6.321 | ATC | 13965 | 5452 |
| 6.322 | ATC | 13966 | 5456 |
| 6.323 | ATC | 13967 | 5459 |
| 6.324 | ATC | 13968 | 5466 |
| 6.325 | ATC | 13969 | 5468 |
| 6.326 | ATC | 13970 | 5469 |
| 6.327 | ATC | 13971 | 5470 |
| 6.329 | ATC | 13972 | 5472 |
| 6.330 | ATC | 13973 | 5473 |

*Spacers having sequence consensus between human and non-human primate genomes

### Transfection of HEK293T Cells:

To assess the editing efficiency of the CasX:gRNA system, HEK293T cells were seeded in a 96-well plate at ~30,000 cells per well in DMEM/F12 medium supplemented with 10% fetal bovine serum. The following day, cells were transfected with the lentiviral vector encoding the CasX variant 491 and gRNA construct using lipofectamine. Two days post-transfection, cells were harvested. A subset of harvested cells was used to determine transfection efficiency by measuring for mScarlet fluorescence using the Attune NxT Flow Cytometer and analyzing the expression via FlowJo, following the manufacturer's instructions. The remaining harvested cells were used for editing analysis by next generation sequencing (NGS).

### NGS Processing and Analysis:

Genomic DNA (gDNA) from harvested cells were extracted using the Zymo Quick-DNA Miniprep Plus kit following the manufacturer's instructions. Target amplicons were formed by amplifying regions of interest from 50 ng of extracted gDNA with a set of primers targeting the human PCSK9 locus. These gene-specific primers contained an additional sequence at the 5′ ends to introduce Illumina reads 1 and 2 sequences. Further, they contained a 16-nucleotide random sequence that functioned as a unique molecular identifier (UMI). The quality and quantification of the amplicon was assessed using a Fragment Analyzer DNA analyzer kit (Agilent, dsDNA 35-1500 bp). Amplicons were sequenced on the Illumina MiSeq™ according to the manufacturer's instructions. Raw fastq sequencing files were processed by trimming for quality and adapter sequences and merging read 1 and read 2 into a single insert sequence; insert sequences were then analyzed by the CRISPResso2 (v 2.0.29) program. The percentage of reads modified in a window around the 3′ end of the spacer was determined. The activity of the CasX molecule was quantified as the total percent of reads that contain insertions, substitutions, and/or deletions anywhere within this window for each.

### Results

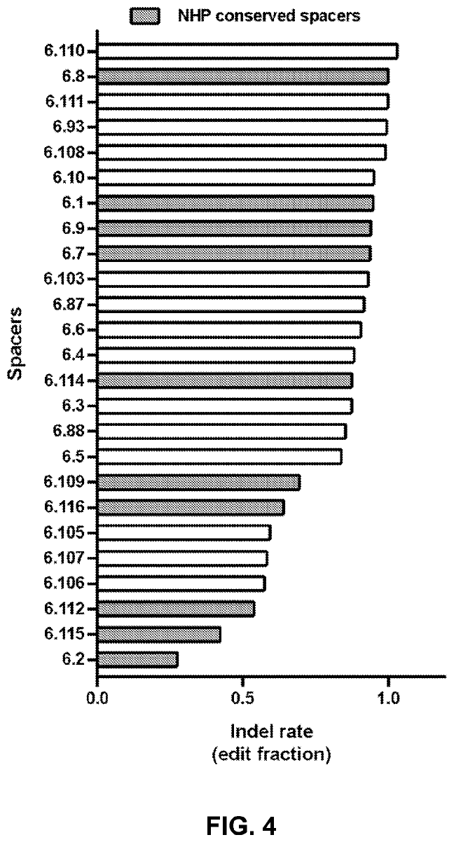

Of the PCSK9-targeting spacers listed in Table 14, 25 spacers were initially assessed for editing efficiency using CasX 491 and gRNA scaffold variant 235, which were expressed from a lentiviral plasmid into HEK293T cells. Editing levels, measured as indel rate detected by NGS for each individual spacer, were normalized by transfection efficiency determined using mScarlet expression. The resulting normalized editing rates, ranked from highest to lowest rates, are illustrated in FIG. 4. The data demonstrate that CasX 491 and gRNAs containing these spacers were able to edit the human PCSK9 locus at varying levels of editing efficiency, ranging from ~20-100% (FIG. 4). Specifically, using 17 out of the 25 spacers resulted in >80% normalized editing rate, demonstrating the robustness of the CasX:gRNA system in targeting the PCSK9 locus in human cells. Notably, spacers 6.8, 6.1, 6.9, 6.7, and 6.114, which achieved >80% normalized editing rate in the editing assay, exhibit sequence conservation between human and non-human primate genomes.

The results demonstrate that CasX and PCSK9-targeting gRNAs can edit the human PCSK9 locus with high efficiency in a cell-based assay. Furthermore, these experiments revealed that several human spacers having consensus sequence with the non-human primate species demonstrated high editing rates, supporting the potential use of these select spacers in preclinical efficacy studies utilizing non-human primate models of familial hypercholesterolemia.

Example 5: Demonstration that CasX:gRNA Systems can Edit the Human PCSK9 Locus to Reduce PCSK9 Secretion In Vitro

Experiments were performed to demonstrate that small CRISPR proteins, such as CasX, and gRNAs, can edit the human PCSK9 locus to reduce secretion of PCSK9 in human HepG2 hepatocytes when packaged and delivered by AAVs.

### Materials and Methods

### AAV Construct Cloning:

CasX variants 491, 668, 672, and 676 and guide scaffold variant 235 were used in these experiments.

AAV constructs (Table 15) containing a UbC promoter driving CasX expression and a Pol III U6 promoter driving gRNA scaffold 235 with a PCSK9-targeting spacer were generated using standard molecular cloning techniques. Spacer 6.8 targeting the human PCSK9 locus was selected for this experiment because it demonstrated the highest editing activity and harbored a consensus sequence with the non-human primate species, as described in Example 3. Cloned and sequence-validated AAV constructs were midi-prepped and subjected to quality assessment prior to transfection into HEK293T cells for AAV production.

TABLE 15
Sequences of AAV constructs assessed for editing at the human PCSK9 locus to
reduce PCSK9 secretion in vitro*

| Construct | Component | | SEQ ID |
|---|---|---|---|
| ID | Name | DNA Sequence | NO: |
| 1 through | AAV2 5′ ITR | CCTGCAGGCAGCTGCGCGCTCGCTCGCTCACTGAGGCCGCCC | 13778 |
| 4 | | GGGCGTCGGGCGACCTTTGGTCGCCCGGCCTCAGTGAGCGAG | |
| | | CGAGCGCGCAGAGAGGGAGTGGCCAACTCCATCACTAGGGGT | |
| | | TCCT | |
| | buffer sequence | GCGGCCTCTAGACTCGAGGCGTT | 13974 |
| | UbC promoter | GGCCTCCGCGCCGGGTTTTGGCGCCTCCCGCGGGCGCCCCCC | 13975 |
| | | TCCTCACGGCGAGCGCTGCCACGTCAGACGAAGGGCGCAGCG | |
| | | AGCGTCCTGATCCTTCCGCCCGGACGCTCAGGACAGCGGCCC | |
| | | GCTGCTCATAAGACTCGGCCTTAGAACCCCAGTATCAGCAGA | |
| | | AGGACATTTTAGGACGGGACTTGGGTGACTCTAGGGCACTGG | |
| | | TTTTCTTTCCAGAGAGCGGAACAGGCGAGGAAAAGTAGTCCC | |
| | | TTCTCGGCGATTCTGCGGAGGGATCTCCGTGGGGCGGTGAAC | |
| | | GCCGATGATTATATAAGGACGCGCCGGGTGTGGCACAGCTAG | |
| | | TTCCGTCGCAGCCGGGATTTGGGTCGCGGTTCTTGTTTGTGG | |
| | | ATCGCTGTGATCGTCACTTGGT | |
| | buffer sequence | CTCTGGCTAACTACCGGT | 13976 |
| | Kozak | GCCACC | 13977 |
| | start codon | ATGGCC | 13978 |
| | cMyc NLS | CCAGCGGCCAAACGGGTGAAGCTGGAC | 13979 |
| | linker | TCTAGA | 13980 |
| 1 | CasX 491 | CAAGAGATCAAGAGAATCAACAAGATCAGAAGGAGACTGGTC | 13981 |
| | | AAGGACAGCAACACAAAGAAGGCCGGCAAGACAGGCCCCATG | |
| | | AAAACCCTGCTCGTCAGAGTGATGACCCCTGACCTGAGAGAG | |
| | | CGGCTGGAAAACCTGAGAAAGAAGCCCGAGAACATCCCTCAG | |
| | | CCTATCAGCAACACCAGCAGGGCCAACCTGAACAAGCTGCTG | |
| | | ACCGACTACACCGAGATGAAGAAAGCCATCCTGCACGTGTAC | |
| | | TGGGAAGAGTTCCAGAAAGACCCCGTGGGCCTGATGAGCAGA | |
| | | GTTGCTCAGCCTGCCAGCAAGAAGATCGACCAGAACAAGCTG | |
| | | AAGCCCGAGATGGACGAGAAGGGCAATCTGACCACAGCCGGC | |
| | | TTTGCCTGCTCTCAGTGTGGCCAGCCTCTGTTCGTGTACAAG | |
| | | CTGGAACAGGTGTCCGAGAAAGGCAAGGCCTACACCAACTAC | |
| | | TTCGGCAGATGTAACGTGGCCGAGCACGAGAAGCTGATTCTG | |
| | | CTGGCCCAGCTGAAACCTGAGAAGGACTCTGATGAGGCCGTG | |
| | | ACCTACAGCCTGGGCAAGTTTGGACAGAGAGCCCTGGACTTC | |
| | | TACAGCATCCACGTGACCAAAGAAAGCACACACCCCGTGAAG | |
| | | CCCCTGGCTCAGATCGCCGGCAATAGATACGCCTCTGGACCT | |
| | | GTGGGCAAAGCCCTGTCCGATGCCTGCATGGGAACAATCGCC | |
| | | AGCTTCCTGAGCAAGTACCAGGACATCATCATCGAGCACCAG | |
| | | AAGGTGGTCAAGGGCAACCAGAAGAGACTGGAAAGCCTGAGG | |
| | | GAGCTGGCCGGCAAAGAGAACCTGGAATACCCCAGCGTGACC | |
| | | CTGCCTCCTCAGCCTCACACAAAAGAAGGCGTGGACGCCTAC | |
| | | AACGAAGTGATCGCCAGAGTGAGAATGTGGGTCAACCTGAAC | |
| | | CTGTGGCAGAAGCTGAAACTGTCCAGGGACGACGCCAAGCCT | |
| | | CTGCTGAGACTGAAGGGCTTCCCTAGCTTCCCTCTGGTGGAA | |
| | | AGACAGGCCAATGAAGTGGATTGGTGGGACATGGTCTGCAAC | |
| | | GTGAAGAAGCTGATCAACGAGAAGAAAGAGGATGGCAAGGTT | |
| | | TTCTGGCAGAACCTGGCCGGCTACAAGAGACAAGAAGCCCTG | |
| | | AGGCCTTACCTGAGCAGCGAAGAGGACCGGAAGAAGGGCAAG | |
| | | AAGTTCGCCAGATACCAGCTGGGCGACCTGCTGCTGCACCTG | |
| | | GAAAAGAAGCACGGCGAGGACTGGGGCAAAGTGTACGATGAG | |
| | | GCCTGGGAGAGAATCGACAAGAAGGTGGAAGGCCTGAGCAAG | |
| | | CACATTAAGCTGGAAGAGGAAAGAAGGAGCGAGGACGCCCAA | |
| | | TCTAAAGCCGCTCTGACCGATTGGCTGAGAGCCAAGGCCAGC | |
| | | TTTGTGATCGAGGGCCTGAAAGAGGCCGACAAGGACGAGTTC | |
| | | TGCAGATGCGAGCTGAAGCTGCAGAAGTGGTACGGCGATCTG | |
| | | AGAGGCAAGCCCTTCGCCATTGAGGCCGAGAACAGCATCCTG | |
| | | GACATCAGCGGCTTCAGCAAGCAGTACAACTGCGCCTTCATT | |
| | | TGGCAGAAAGACGGCGTCAAGAAACTGAACCTGTACCTGATC | |
| | | ATCAATTACTTCAAAGGCGGCAAGCTGCGGTTCAAGAAGATC | |
| | | AAACCCGAGGCCTTCGAGGCTAACAGATTCTACACCGTGATC | |
| | | AACAAAAAGTCCGGCGAGATCGTGCCCATGGAAGTGAACTTC | |
| | | AACTTCGACGACCCCAACCTGATTATCCTGCCTCTGGCCTTC | |
| | | GGCAAGAGACAGGGCAGAGAGTTCATCTGGAACGATCTGCTG | |
| | | AGCCTGGAAACCGGCTCTCTGAAGCTGGCCAATGGCAGAGTG | |
| | | ATCGAGAAAACCCTGTACAACAGGAGAACCAGACAGGACGAG | |
| | | CCTGCTCTGTTTGTGGCCCTGACCTTCGAGAGAAGAGAGGTG | |
| | | CTGGACAGCAGCAACATCAAGCCCATGAACCTGATCGGCGTG | |
| | | GACCGGGGCGAGAATATCCCTGCTGTGATCGCCCTGACAGAC | |
| | | CCTGAAGGATGCCCACTGAGCAGATTCAAGGACTCCCTGGGC | |
| | | AACCCTACACACATCCTGAGAATCGGCGAGAGCTACAAAGAG | |
| | | AAGCAGAGGACAATCCAGGCCAAGAAAGAGGTGGAACAGCGC | |
| | | AGAGCCGGCGGATACTCTAGGAAGTACGCCAGCAAGGCCAAG | |
| | | AATCTGGCCGACGACATGGTCCGAAACACCGCCAGAGATCTG | |
| | | CTGTACTACGCCGTGACACAGGACGCCATGCTGATCTTCGAG | |
| | | AATCTGAGCAGAGGCTTCGGCCGGCAGGGCAAGAGAACCTTT | |
| | | ATGGCCGAGAGGCAGTACACCAGAATGGAAGATTGGCTCACA | |
| | | GCTAAACTGGCCTACGAGGGACTGAGCAAGACCTACCTGTCC | |
| | | AAAACACTGGCCCAGTATACCTCCAAGACCTGCAGCAATTGC | |
| | | GGCTTCACCATCACCAGCGCCGACTACGACAGAGTGCTGGAA | |
| | | AAGCTCAAGAAAACCGCCACCGGCTGGATGACCACCATCAAC | |
| | | GGCAAAGAGCTGAAGGTTGAGGGCCAGATCACCTACTACAAC | |
| | | AGGTACAAGAGGCAGAACGTCGTGAAGGATCTGAGCGTGGAA | |
| | | CTGGACAGACTGAGCGAAGAGAGCGTGAACAACGACATCAGC | |
| | | AGCTGGACAAAGGGCAGATCAGGCGAGGCTCTGAGCCTGCTG | |
| | | AAGAAGAGGTTTAGCCACAGACCTGTGCAAGAGAAGTTCGTG | |
| | | TGCCTGAACTGCGGCTTCGAGACACACGCCGATGAACAGGCT | |
| | | GCCCTGAACATTGCCAGAAGCTGGCTGTTCCTGAGAAGCCAA | |
| | | GAGTACAAGAAGTACCAGACCAACAAGACCACCGGCAACACC | |
| | | GACAAGAGGGCCTTTGTGGAAACCTGGCAGAGCTTCTACAGA | |
| | | AAAAAGCTGAAAGAAGTCTGGAAGCCCGCCGTG | |
| 2 | CasX 668 | CAAGAGATCAAGAGAATCAACAAGATCAGAAGGAGACTGGTC | 13982 |
| | | AAGGACAGCAACACAAAGAAGGCCGGCAAGACACGGGGCCCC | |
| | | ATGAAAACCCTGCTCGTCAGAGTGATGACCCCTGACCTGAGA | |
| | | GAGCGGCTGGAAAACCTGAGAAAGAAGCCCGAGAACATCCCT | |
| | | CAGCCTATCAGCAACACCAGCAGGGCCAACCTGAACAAGCTG | |
| | | CTGACCGACTACACCGAGATGAAGAAAGCCATCCTGCACGTG | |
| | | TACTGGGAAGAGTTCCAGAAAGACCCCGTGGGCCTGATGAGC | |
| | | AGAGTTGCTCAGCCTGCCAGCAAGAAGATCGACCAGAACAAG | |
| | | CTGAAGCCCGAGATGGACGAGAAGGGCAATCTGACCACAGCC | |
| | | GGCTTTGCCTGCTCTCAGTGTGGCCAGCCTCTGTTCGTGTAC | |
| | | AAGCTGGAACAGGTGTCCGAGAAAGGCAAGGCCTACACCAAC | |
| | | TACTTCGGCAGATGTAACGTGGCCGAGCACGAGAAGCTGATT | |
| | | CTGCTGGCCCAGCTGAAACCTGAGAAGGACTCTGATGAGGCC | |
| | | GTGACCTACAGCCTGGGCAAGTTTGGACAGAGAGCCCTGGAC | |
| | | TTCTACAGCATCCACGTGACCAAAGAAAGCACACACCCCGTG | |
| | | AAGCCCCTGGCTCAGATCGCCGGCAATAGATACGCCTCTTCC | |
| | | CCTGTGGGCAAAGCCCTGTCCGATGCCTGCATGGGAACAATC | |
| | | GCCAGCTTCCTGAGCAAGTACCAGGACATCATCATCGAGCAC | |
| | | CAGAAGGTGGTCAAGGGCAACCAGAAGAGACTGGAAAGCCTG | |
| | | AGGGAGCTGGCCGGCAAAGAGAACCTGGAATACCCCAGCGTG | |
| | | ACCCTGCCTCCTCAGCCTCACACAAAAGAAGGCGTGGACGCC | |
| | | TACAACGAAGTGATCGCCAGAGTGAGAATGTGGGTCAACCTG | |
| | | AACCTGTGGCAGAAGCTGAAACTGTCCAGGGACGACGCCAAG | |
| | | CCTCTGCTGAGACTGAAGGGCTTCCCTAGCTTCCCTCTGGTG | |
| | | GAAAGACAGGCCAATGAAGTGGATTGGTGGGACATGGTCTGC | |
| | | AACGTGAAGAAGCTGATCAACGAGAAGAAAGAGGATGGCAAG | |
| | | GTTTTCTGGCAGAACCTGGCCGGCTACAAGAGACAAGAAGCC | |
| | | CTGAGGCCTTACCTGAGCAGCGAAGAGGACCGGAAGAAGGGC | |
| | | AAGAAGTTCGCCAGATACCAGCTGGGCGACCTGCTGCTGCAC | |
| | | CTGGAAAAGAAGCACGGCGAGGACTGGGGCAAAGTGTACGAT | |
| | | GAGGCCTGGGAGAGAATCGACAAGAAGGTGGAAGGCCTGAGC | |
| | | AAGCACATTAAGCTGGAAGAGGAAAGAAGGAGCGAGGACGCC | |
| | | CAATCTAAAGCCGCTCTGACCGATTGGCTGAGAGCCAAGGCC | |
| | | AGCTTTGTGATCGAGGGCCTGAAAGAGGCCGACAAGGACGAG | |
| | | TTCTGCAGATGCGAGCTGAAGCTGCAGAAGTGGTACGGCGAT | |
| | | CTGAGAGGCAAGCCCTTCGCCATTGAGGCCGAGAACAGCATC | |
| | | CTGGACATCAGCGGCTTCAGCAAGCAGTACAACTGCGCCTTC | |
| | | ATTTGGCAGAAAGACGGCGTCAAGAAACTGAACCTGTACCTG | |
| | | ATCATCAATTACTTCAAAGGCGGCAAGCTGCGGTTCAAGAAG | |
| | | ATCAAACCCGAGGCCTTCGAGGCTAACAGATTCTACACCGTG | |
| | | ATCAACAAAAAGTCCGGCGAGATCGTGCCCATGGAAGTGAAC | |
| | | TTCAACTTCGACGACCCCAACCTGATTATCCTGCCTCTGGCC | |
| | | TTCGGCAAGAGACAGGGCAGAGAGTTCATCTGGAACGATCTG | |
| | | CTGAGCCTGGAAACCGGCTCTCTGAAGCTGGCCAATGGCAGA | |
| | | GTGATCGAGAAAACCCTGTACAACAGGAGAACCAGACAGGAC | |
| | | GAGCCTGCTCTGTTTGTGGCCCTGACCTTCGAGAGAAGAGAG | |
| | | GTGCTGGACAGCAGCAACATCAAGCCCATGAACCTGATCGGC | |
| | | GTGGACCGGGGCGAGAATATCCCTGCTGTGATCGCCCTGACA | |
| | | GACCCTGAAGGATGCCCACTGAGCAGATTCAAGGACTCCCTG | |
| | | GGCAACCCTACACACATCCTGAGAATCGGCGAGAGCTACAAA | |
| | | GAGAAGCAGAGGACAATCCAGGCCAAGAAAGAGGTGGAACAG | |
| | | AGAAGAGCCGGCGGATACTCTAGGAAGTACGCCAGCAAGGCC | |
| | | AAGAATCTGGCCGACGACATGGTCCGAAACACCGCCAGAGAT | |
| | | CTGCTGTACTACGCCGTGACACAGGACGCCATGCTGATCTTC | |
| | | GAGAATCTGAGCAGAGGCTTCGGCCGGCAGGGCAAGAGAACC | |
| | | TTTATGGCCGAGAGGCAGTACACCAGAATGGAAGATTGGCTC | |
| | | ACAGCTAAACTGGCCTACGAGGGACTGCCCAGCAAGACCTAC | |
| | | CTGTCCAAAACACTGGCCCAGTATACCTCCAAGACCTGCAGC | |
| | | AATTGCGGCTTCACCATCACCAGCGCCGACTACGACAGAGTG | |
| | | CTGGAAAAGCTCAAGAAAACCGCCACCGGCTGGATGACCACC | |
| | | ATCAACGGCAAAGAGCTGAAGGTTGAGGGCCAGATCACCTAC | |
| | | TACAACAGGTACAAGAGGCAGAACGTCGTGAAGGATCTGAGC | |
| | | GTGGAACTGGACAGACTGAGCGAAGAGAGCGTGAACAACGAC | |
| | | ATCAGCAGCTGGACAAAGGGCAGATCAGGCGAGGCTCTGAGC | |
| | | CTGCTGAAGAAGAGGTTTAGCCACAGACCTGTGCAAGAGAAG | |
| | | TTCGTGTGCCTGAACTGCGGCTTCGAGACACACGCCGATGAA | |
| | | CAGGCTGCCCTGAACATTGCCAGAAGCTGGCTGTTCCTGAGA | |
| | | AGCCAAGAGTACAAGAAGTACCAGACCAACAAGACCACCGGC | |
| | | AACACCGACAAGAGGGCCTTTGTGGAAACCTGGCAGAGCTTC | |
| | | TACAGAAAAAAGCTGAAAGAAGTCTGGAAGCCCGCCGTG | |
| 3 | CasX 672 | CAAGAGATCAAGAGAATCAACAAGATCAGAAGGAGACTGGTC | 13983 |
| | | AAGGACAGCAACACAAAGAAGGCCGGCAAGACAGGCCCCATG | |
| | | AAAACCCTGCTCGTCAGAGTGATGACCCCTGACCTGAGAGAG | |
| | | CGGCTGGAAAACCTGAGAAAGAAGCCCGAGAACATCCCTCAG | |
| | | CCTATCAGCAACACCAGCAGGGCCAACCTGAACAAGCTGCTG | |
| | | ACCGACTACACCGAGATGAAGAAAGCCATCCTGCACGTGTAC | |
| | | TGGGAAGAGTTCCAGAAAGACCCCGTGGGCCTGATGAGCAGA | |
| | | GTTGCTCAGCCTGCCAGCAAGAAGATCGACCAGAACAAGCTG | |
| | | AAGCCCGAGATGGACGAGAAGGGCAATCTGACCACAGCCGGC | |
| | | TTTGCCTGCTCTCAGTGTGGCCAGCCTCTGTTCGTGTACAAG | |
| | | CTGGAACAGGTGTCCGAGAAAGGCAAGGCCTACACCAACTAC | |
| | | TTCGGCAGATGTAACGTGGCCGAGCACGAGAAGCTGATTAAG | |
| | | CTGGCCCAGCTGAAACCTGAGAAGGACTCTGATGAGGCCGTG | |
| | | ACCTACAGCCTGGGCAAGTTTGGACAGAGAGCCCTGGACTTC | |
| | | TACAGCATCCACGTGACCAAAGAAAGCACACACCCCGTGAAG | |
| | | CCCCTGGCTCAGATCGCCGGCAATAGATACGCCTCTTCCCCT | |
| | | GTGGGCAAAGCCCTGTCCGATGCCTGCATGGGAACAATCGCC | |
| | | AGCTTCCTGAGCAAGTACCAGGACATCATCATCGAGCACCAG | |
| | | AAGGTGGTCAAGGGCAACCAGAAGAGACTGGAAAGCCTGAGG | |
| | | GAGCTGGCCGGCAAAGAGAACCTGGAATACCCCAGCGTGACC | |
| | | CTGCCTCCTCAGCCTCACACAAAAGAAGGCGTGGACGCCTAC | |
| | | AACGAAGTGATCGCCAGAGTGAGAATGTGGGTCAACCTGAAC | |
| | | CTGTGGCAGAAGCTGAAACTGTCCAGGGACGACGCCAAGCCT | |
| | | CTGCTGAGACTGAAGGGCTTCCCTAGCTTCCCTCTGGTGGAA | |
| | | AGACAGGCCAATGAAGTGGATTGGTGGGACATGGTCTGCAAC | |
| | | GTGAAGAAGCTGATCAACGAGAAGAAAGAGGATGGCAAGGTT | |
| | | TTCTGGCAGAACCTGGCCGGCTACAAGAGACAAGAAGCCCTG | |
| | | AGGCCTTACCTGAGCAGCGAAGAGGACCGGAAGAAGGGCAAG | |
| | | AAGTTCGCCAGATACCAGCTGGGCGACCTGCTGCTGCACCTG | |
| | | GAAAAGAAGCACGGCGAGGACTGGGGCAAAGTGTACGATGAG | |
| | | GCCTGGGAGAGAATCGACAAGAAGGTGGAAGGCCTGAGCAAG | |
| | | CACATTAAGCTGGAAGAGGAAAGAAGGAGCGAGGACGCCCAA | |
| | | TCTAAAGCCGCTCTGACCGATTGGCTGAGAGCCAAGGCCAGC | |
| | | TTTGTGATCGAGGGCCTGAAAGAGGCCGACAAGGACGAGTTC | |
| | | TGCAGATGCGAGCTGAAGCTGCAGAAGTGGTACGGCGATCTG | |
| | | AGAGGCAAGCCCTTCGCCATTGAGGCCGAGAACAGCATCCTG | |
| | | GACATCAGCGGCTTCAGCAAGCAGTACAACTGCGCCTTCATT | |
| | | TGGCAGAAAGACGGCGTCAAGAAACTGAACCTGTACCTGATC | |
| | | ATCAATTACTTCAAAGGCGGCAAGCTGCGGTTCAAGAAGATC | |
| | | AAACCCGAGGCCTTCGAGGCTAACAGATTCTACACCGTGATC | |
| | | AACAAAAAGTCCGGCGAGATCGTGCCCATGGAAGTGAACTTC | |
| | | AACTTCGACGACCCCAACCTGATTATCCTGCCTCTGGCCTTC | |
| | | GGCAAGAGACAGGGCAGAGAGTTCATCTGGAACGATCTGCTG | |
| | | AGCCTGGAAACCGGCTCTCTGAAGCTGGCCAATGGCAGAGTG | |
| | | ATCGAGAAAACCCTGTACAACAGGAGAACCAGACAGGACGAG | |
| | | CCTGCTCTGTTTGTGGCCCTGACCTTCGAGAGAAGAGAGGTG | |
| | | CTGGACAGCAGCAACATCAAGCCCATGAACCTGATCGGCGTG | |
| | | GACCGGGGCGAGAATATCCCTGCTGTGATCGCCCTGACAGAC | |
| | | CCTGAAGGATGCCCACTGAGCAGATTCAAGGACTCCCTGGGC | |
| | | AACCCTACACACATCCTGAGAATCGGCGAGAGCTACAAAGAG | |
| | | AAGCAGAGGACAATCCAGGCCAAGAAAGAGGTGGAACAGAGA | |
| | | AGAGCCGGCGGATACTCTAGGAAGTACGCCAGCAAGGCCAAG | |
| | | AATCTGGCCGACGACATGGTCCGAAACACCGCCAGAGATCTG | |
| | | CTGTACTACGCCGTGACACAGGACGCCATGCTGATCTTCGAG | |
| | | AATCTGAGCAGAGGCTTCGGCCGGCAGGGCAAGAGAACCTTT | |
| | | ATGGCCGAGAGGCAGTACACCAGAATGGAAGATTGGCTCACA | |
| | | GCTAAACTGGCCTACGAGGGACTGCCCAGCAAGACCTACCTG | |
| | | TCCAAAACACTGGCCCAGTATACCTCCAAGACCTGCAGCAAT | |
| | | TGCGGCTTCACCATCACCAGCGCCGACTACGACAGAGTGCTG | |
| | | GAAAAGCTCAAGAAAACCGCCACCGGCTGGATGACCACCATC | |
| | | AACGGCAAAGAGCTGAAGGTTGAGGGCCAGATCACCTACTAC | |
| | | AACAGGTACAAGAGGCAGAACGTCGTGAAGGATCTGAGCGTG | |
| | | GAACTGGACAGACTGAGCGAAGAGAGCGTGAACAACGACATC | |
| | | AGCAGCTGGACAAAGGGCAGATCAGGCGAGGCTCTGAGCCTG | |
| | | CTGAAGAAGAGGTTTAGCCACAGACCTGTGCAAGAGAAGTTC | |
| | | GTGTGCCTGAACTGCGGCTTCGAGACACACGCCGATGAACAG | |
| | | GCTGCCCTGAACATTGCCAGAAGCTGGCTGTTCCTGAGAAGC | |
| | | CAAGAGTACAAGAAGTACCAGACCAACAAGACCACCGGCAAC | |
| | | ACCGACAAGAGGGCCTTTGTGGAAACCTGGCAGAGCTTCTAC | |
| | | AGAAAAAAGCTGAAAGAAGTCTGGAAGCCCGCCGTG | |
| 4 | CasX 676 | CAAGAGATCAAGAGAATCAACAAGATCAGAAGGAGACTGGTC | 13984 |
| | | AAGGACAGCAACACAAAGAAGGCCGGCAAGACACGGGGCCCC | |
| | | ATGAAAACCCTGCTCGTCAGAGTGATGACCCCTGACCTGAGA | |
| | | GAGCGGCTGGAAAACCTGAGAAAGAAGCCCGAGAACATCCCT | |
| | | CAGCCTATCAGCAACACCAGCAGGGCCAACCTGAACAAGCTG | |
| | | CTGACCGACTACACCGAGATGAAGAAAGCCATCCTGCACGTG | |
| | | TACTGGGAAGAGTTCCAGAAAGACCCCGTGGGCCTGATGAGC | |
| | | AGAGTTGCTCAGCCTGCCAGCAAGAAGATCGACCAGAACAAG | |
| | | CTGAAGCCCGAGATGGACGAGAAGGGCAATCTGACCACAGCC | |
| | | GGCTTTGCCTGCTCTCAGTGTGGCCAGCCTCTGTTCGTGTAC | |
| | | AAGCTGGAACAGGTGTCCGAGAAAGGCAAGGCCTACACCAAC | |
| | | TACTTCGGCAGATGTAACGTGGCCGAGCACGAGAAGCTGATT | |
| | | AAGCTGGCCCAGCTGAAACCTGAGAAGGACTCTGATGAGGCC | |
| | | GTGACCTACAGCCTGGGCAAGTTTGGACAGAGAGCCCTGGAC | |
| | | TTCTACAGCATCCACGTGACCAAAGAAAGCACACACCCCGTG | |
| | | AAGCCCCTGGCTCAGATCGCCGGCAATAGATACGCCTCTTCC | |
| | | CCTGTGGGCAAAGCCCTGTCCGATGCCTGCATGGGAACAATC | |
| | | GCCAGCTTCCTGAGCAAGTACCAGGACATCATCATCGAGCAC | |
| | | CAGAAGGTGGTCAAGGGCAACCAGAAGAGACTGGAAAGCCTG | |
| | | AGGGAGCTGGCCGGCAAAGAGAACCTGGAATACCCCAGCGTG | |
| | | ACCCTGCCTCCTCAGCCTCACACAAAAGAAGGCGTGGACGCC | |
| | | TACAACGAAGTGATCGCCAGAGTGAGAATGTGGGTCAACCTG | |
| | | AACCTGTGGCAGAAGCTGAAACTGTCCAGGGACGACGCCAAG | |
| | | CCTCTGCTGAGACTGAAGGGCTTCCCTAGCTTCCCTCTGGTG | |
| | | GAAAGACAGGCCAATGAAGTGGATTGGTGGGACATGGTCTGC | |
| | | AACGTGAAGAAGCTGATCAACGAGAAGAAAGAGGATGGCAAG | |
| | | GTTTTCTGGCAGAACCTGGCCGGCTACAAGAGACAAGAAGCC | |
| | | CTGAGGCCTTACCTGAGCAGCGAAGAGGACCGGAAGAAGGGC | |
| | | AAGAAGTTCGCCAGATACCAGCTGGGCGACCTGCTGCTGCAC | |
| | | CTGGAAAAGAAGCACGGCGAGGACTGGGGCAAAGTGTACGAT | |
| | | GAGGCCTGGGAGAGAATCGACAAGAAGGTGGAAGGCCTGAGC | |
| | | AAGCACATTAAGCTGGAAGAGGAAAGAAGGAGCGAGGACGCC | |
| | | CAATCTAAAGCCGCTCTGACCGATTGGCTGAGAGCCAAGGCC | |
| | | AGCTTTGTGATCGAGGGCCTGAAAGAGGCCGACAAGGACGAG | |
| | | TTCTGCAGATGCGAGCTGAAGCTGCAGAAGTGGTACGGCGAT | |
| | | CTGAGAGGCAAGCCCTTCGCCATTGAGGCCGAGAACAGCATC | |
| | | CTGGACATCAGCGGCTTCAGCAAGCAGTACAACTGCGCCTTC | |
| | | ATTTGGCAGAAAGACGGCGTCAAGAAACTGAACCTGTACCTG | |
| | | ATCATCAATTACTTCAAAGGCGGCAAGCTGCGGTTCAAGAAG | |
| | | ATCAAACCCGAGGCCTTCGAGGCTAACAGATTCTACACCGTG | |
| | | ATCAACAAAAAGTCCGGCGAGATCGTGCCCATGGAAGTGAAC | |
| | | TTCAACTTCGACGACCCCAACCTGATTATCCTGCCTCTGGCC | |
| | | TTCGGCAAGAGACAGGGCAGAGAGTTCATCTGGAACGATCTG | |
| | | CTGAGCCTGGAAACCGGCTCTCTGAAGCTGGCCAATGGCAGA | |
| | | GTGATCGAGAAAACCCTGTACAACAGGAGAACCAGACAGGAC | |
| | | GAGCCTGCTCTGTTTGTGGCCCTGACCTTCGAGAGAAGAGAG | |
| | | GTGCTGGACAGCAGCAACATCAAGCCCATGAACCTGATCGGC | |
| | | GTGGACCGGGGCGAGAATATCCCTGCTGTGATCGCCCTGACA | |
| | | GACCCTGAAGGATGCCCACTGAGCAGATTCAAGGACTCCCTG | |
| | | GGCAACCCTACACACATCCTGAGAATCGGCGAGAGCTACAAA | |
| | | GAGAAGCAGAGGACAATCCAGGCCAAGAAAGAGGTGGAACAG | |
| | | AGAAGAGCCGGCGGATACTCTAGGAAGTACGCCAGCAAGGCC | |
| | | AAGAATCTGGCCGACGACATGGTCCGAAACACCGCCAGAGAT | |
| | | CTGCTGTACTACGCCGTGACACAGGACGCCATGCTGATCTTC | |
| | | GAGAATCTGAGCAGAGGCTTCGGCCGGCAGGGCAAGAGAACC | |
| | | TTTATGGCCGAGAGGCAGTACACCAGAATGGAAGATTGGCTC | |
| | | ACAGCTAAACTGGCCTACGAGGGACTGCCCAGCAAGACCTAC | |
| | | CTGTCCAAAACACTGGCCCAGTATACCTCCAAGACCTGCAGC | |
| | | AATTGCGGCTTCACCATCACCAGCGCCGACTACGACAGAGTG | |
| | | CTGGAAAAGCTCAAGAAAACCGCCACCGGCTGGATGACCACC | |
| | | ATCAACGGCAAAGAGCTGAAGGTTGAGGGCCAGATCACCTAC | |
| | | TACAACAGGTACAAGAGGCAGAACGTCGTGAAGGATCTGAGC | |
| | | GTGGAACTGGACAGACTGAGCGAAGAGAGCGTGAACAACGAC | |
| | | ATCAGCAGCTGGACAAAGGGCAGATCAGGCGAGGCTCTGAGC | |
| | | CTGCTGAAGAAGAGGTTTAGCCACAGACCTGTGCAAGAGAAG | |
| | | TTCGTGTGCCTGAACTGCGGCTTCGAGACACACGCCGATGAA | |
| | | CAGGCTGCCCTGAACATTGCCAGAAGCTGGCTGTTCCTGAGA | |
| | | AGCCAAGAGTACAAGAAGTACCAGACCAACAAGACCACCGGC | |
| | | AACACCGACAAGAGGGCCTTTGTGGAAACCTGGCAGAGCTTC | |
| | | TACAGAAAAAAGCTGAAAGAAGTCTGGAAGCCCGCCGTG | |
| 1 through | linker | GGATCC | 13985 |
| 4 | cMyc NLS | CCAGCCGCGAAGCGAGTGAAACTGGAC | 13986 |
| | stop codon + | TAAGAATTCCTAGAGCTCGCTGATCAGCCTCGA | 13987 |
| | buffer sequence | | |
| | bGH poly(A) | CTGTGCCTTCTAGTTGCCAGCCATCTGTTGTTTGCCCCTCCC | 13988 |
| | | CCGTGCCTTCCTTGACCCTGGAAGGTGCCACTCCCACTGTCC | |
| | | TTTCCTAATAAAATGAGGAAATTGCATCGCATTGTCTGAGTA | |
| | | GGTGTCATTCTATTCTGGGGGGTGGGGTGGGGCAGGACAGCA | |
| | | AGGGGGAGGATTGGGAAGAGAATAGCAGGCATGCTGGGGA | |
| | buffer sequence | GGTACC | 13989 |
| | U6 promoter | GAGGGCCTATTTCCCATGATTCCTTCATATTTGCATATACGA | 13990 |
| | | TACAAGGCTGTTAGAGAGATAATTGGAATTAATTTGACTGTA | |
| | | AACACAAAGATATTAGTACAAAATACGTGACGTAGAAAGTAA | |
| | | TAATTTCTTGGGTAGTTTGCAGTTTTAAAATTATGTTTTAAA | |
| | | ATGGACTATCATATGCTTACCGTAACTTGAAAGTATTTCGAT | |
| | | TTCTTGGCTTTATATATCTTGTGGAAAGGAC | |
| | buffer sequence | GAAACACC | 13991 |
| | Scaffold 235 | ACTGGCGCTTCTATCTGATTACTCTGAGCGCCATCACCAGCG | 13992 |
| | | ACTATGTCGTAGTGGGTAAAGCCGCTTACGGACTTCGGTCCG | |
| | | TAAGAGGCATCAGAG | |
| | Spacer 6.8 | TGGCTTCCTGGTGAAGATGA | 13789 |
| | buffer sequence | TTTTTTTTGGCGGCCGC | 13993 |
| | AAV2 3′ ITR | AGGAACCCCTAGTGATGGAGTTGGCCACTCCCTCTCTGCGCG | 13779 |
| | | CTCGCTCGCTCACTGAGGCCGGGCGACCAAAGGTCGCCCGAC | |
| | | GCCCGGGCTTTGCCCGGGCGGCCTCAGTGAGCGAGCGAGCGC | |
| | | GCAGCTGCCTGCAGG | |

*Components are listed in a 5′ to 3′ order within constructs 1-4

### AAV Production:

Suspension-adapted HEK293T cells were seeded in 20-30 mL of media at 1.5E6 cells/mL on the day of transfection. AAV plasmids with the transgene flanked by ITR repeats were co-transfected with plasmids supplying the adenoviral helper genes for replication and AAV rep/cap genome using PEI Max (Polysciences) in serum-free Opti-MEM media. Three days post-transfection, cultures were centrifuged to separate the supernatant from the cell pellet, and the AAV particles were collected, concentrated, and filtered following standard procedures.

To determine the viral genome (vg) titer, 1 μL from crude lysate virus was digested with DNase and ProtK, followed by quantitative PCR. 5 μL of digested virus was used in a 25 μL qPCR reaction composed of IDT primetime master mix and a set of primer and 6′FAM/Zen/IBFQ probe (IDT) designed to amplify the CMV promoter region or a 62 bp-fragment located in the AAV2-ITR. Ten-fold serial dilutions of an AAV ITR plasmid was used as reference standards to calculate the titer (vg/mL) of viral samples.

### In Vitro AAV Transduction of Human HepG2 Hepatocytes:

~30,000 HepG2 cells were seeded per well in a collagen coated 96-well plate. 24 hours later, seeded cells were transduced with AAVs encoding a CasX variant and a gRNA with scaffold 235 and spacer 6.8 (Table 15) at a multiplicity of infection (MOI) of 7E5 vg/cell, using two wells per construct as replicates. Here, untreated cells served as a negative control. Six days after transduction, the following were harvested: 1) transduced cells were harvested for editing assessment at the PCSK9 locus by NGS; 2) media supernatant was harvested to measure secreted PCSK9 protein levels by ELISA. For editing analysis by NGS, amplicons were amplified from 50 ng of extracted gDNA with a set of primers targeting the PCSK9 locus and processed as described earlier in Example 3. Secreted PCSK9 levels in the media supernatant were also analyzed using a fluorescence resonance energy transfer-based immunoassay from CISBio following the manufacturer's instructions.

### Results

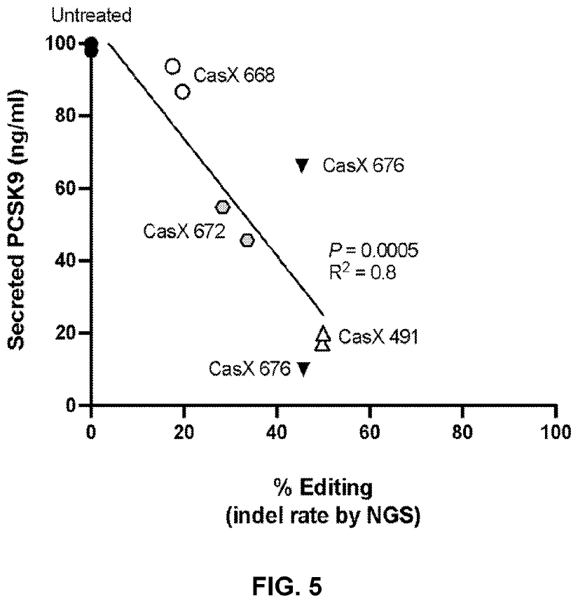

CasX variants 491, 668, 672, and 676 were assessed for their editing efficiency at the human PCSK9 locus when expressed from the AAV episome delivered by AAVs used to transduce HepG2 cells. The plot in FIG. 5 shows an inverse correlation between the editing events at the PCSK9 locus and levels of PCSK9 secretion in HepG2 cells transduced in vitro. The data demonstrate that with the PCSK9-targeting gRNA, CasX 491 (n=2) and CasX 676 (n=1) were able to edit the PCSK9 locus with ~50% editing efficiency, while CasX 668 (n=2) and CasX 672 (n=2) were able to edit with ~20% and ~30% editing efficiency respectively (FIG. 5). The ~50% editing at the PCSK9 locus exhibited by CasX 491 and CasX 676 correlated with the effective decrease in secreted PCSK9 levels by ~80%, a therapeutically relevant threshold, relative to secreted PCSK9 levels in the untreated control. Meanwhile the lower editing efficiency achieved by CasX 668 and CasX 672 resulted in correspondingly lower reduction (~10% and ~50% respectively) in PCSK9 secretion (FIG. 5).

The results demonstrate that CasX variants and PCSK9-targeting gRNAs can edit the human PCSK9 locus with efficiency to achieve a therapeutically relevant reduction of PCSK9 secretion in a cell-based assay. Furthermore, these findings show that AAV delivery of the CasX:gRNA system is attainable to induce effective editing at the PCSK9 locus.

Example 6: Design and Assessment of Modified gRNAs when Delivered Together with CasX mRNA In Vitro and In Vivo

Experiments were performed to identify new gRNA variant sequences and demonstrate that chemical modifications of these gRNA variants enhance the editing efficiency of the CasX:gRNA system when delivered in vitro in conjunction with CasX mRNA.

### Materials and Methods

Synthesis of gRNAs:

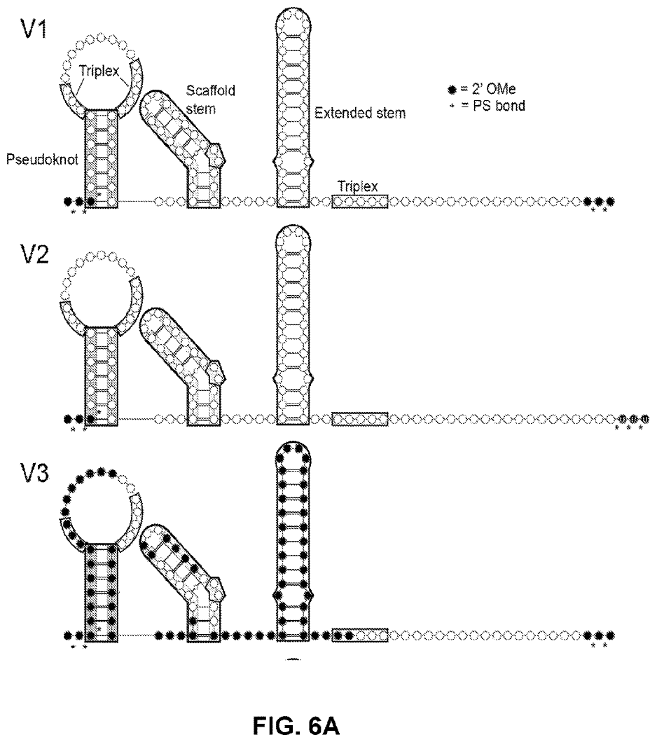

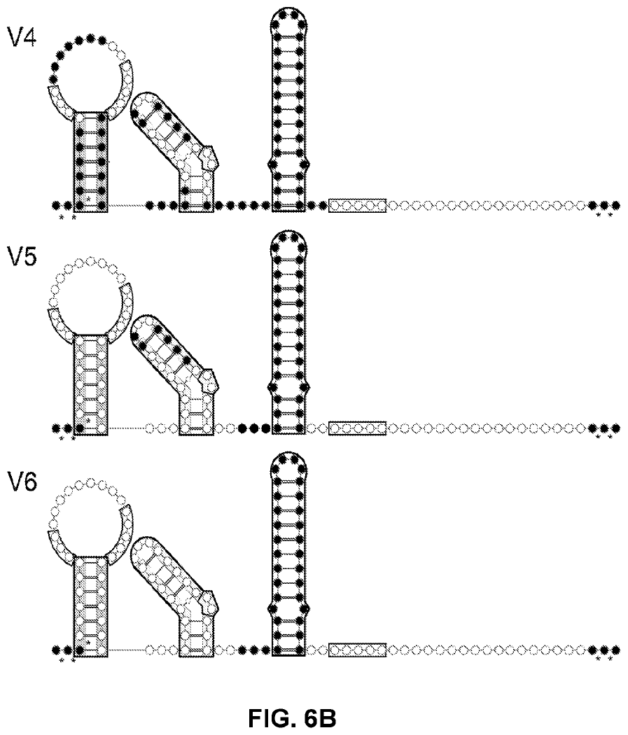

All gRNAs tested in this example were chemically-synthesized and were derived from gRNA scaffolds 174, 235, and 316. The sequences of gRNA scaffolds 174, 235, and 316 and their chemical modification profiles are listed in Table 16. The sequences of the resulting gRNAs, including spacers targeting PCSK9, B2M, or ROSA26, and their chemical modification profiles assayed in this example are listed in Table 17. A schematic of the structure of gRNA scaffold variants 174, 235, and 316 are shown in FIGS. 9A-9C, respectively, and the sites of chemical modifications of the gRNA variants are shown schematically in FIGS. 6A, 6B, 8, 14A, and 14B.

TABLE 16
Sequences of gRNA scaffolds with their different chemical modification profiles
(denoted by version number), where “NNNNNNNNNNNNNNNNNNNN” is a spacer
placeholder. Chemical modifications: * = phosphorothioate bond; m = 2′OMe
modification

| gRNA | | |
|---|---|---|
| scaffold | | SEQ |
| (version) | gRNA sequence | ID NO: |
| 174 (v0) | ACUGGCGCUUUUAUCUGAUUACUUUGAGAGCCAUCACCAGCGACUAUGU | 13748 |
| | CGUAGUGGGUAAAGCUCCCUCUUCGGAGGGAGCAUCAAAGNNNNNNNNN | |
| | NNNNNNNNNNN | |
| 174 (v1) | mA*mC*mU*GGCGCUUUUAUCUGAUUACUUUGAGAGCCAUCACCAGCGA | 13749 |
| | CUAUGUCGUAGUGGGUAAAGCUCCCUCUUCGGAGGGAGCAUCAAAGNNN | |
| | NNNNNNNNNNNNNNmN*mN*mN | |
| 174 (v2) | mA*mC*mU*GGCGCUUUUAUCUGAUUACUUUGAGAGCCAUCACCAGCGA | 13750 |
| | CUAUGUCGUAGUGGGUAAAGCUCCCUCUUCGGAGGGAGCAUCAAAGNNN | |
| | NNNNNNNNNNNNNNNNN*mU*mU*mU | |
| 174 (v3) | mA*mC*mU*mGmGmCmGmCmUmUmUmUmAmUmCmUmGmAmUUACUUUGm | 13751 |
| | AmGmAmGmCmCmAmUmCmAmCmCAGCGAmCmUAUmGmUmCmGUAGUGmG | |
| | mGmUmAmAmAmGmCmUmCmCmCmUmCmUmUmCmGmGmAmGmGmGmAmGm | |
| | CmAmUmCmAAAGNNNNNNNNNNNNNNNNNmN*mN*mN | |
| 174 (v4) | mA*mC*mU*mGmGmCmGmCUUUUmAmUmCmUmGmAmUUACUUUGmAmGm | 13752 |
| | AmGmCmCmAmUmCmAmCmCAGCGAmCmUAUmGmUmCmGUAGUGmGmGmU | |
| | mAmAmAmGmCmUmCmCmCmUmCmUmUmCmGmGmAmGmGmGmAmGmCmAm | |
| | UCAAAGNNNNNNNNNNNNNNNNNmN*mN*mN | |
| 174 (v5) | mA*mC*mU*GGCGCUUUUAUCUGAUUACUUUGAGAGCCAUCACCAGCGA | 13753 |
| | mCmUAUmGmUmCmGUAGUGGGUAAAmGmCmUmCmCmCmUmCmUmUmCmG | |
| | mGmAmGmGmGmAmGmCAUCAAAGNNNNNNNNNNNNNNNNNmN*mN*mN | |
| 174 (v6) | mA*mC*mU*GGCGCUUUUAUCUGAUUACUUUGAGAGCCAUCACCAGCGA | 13754 |
| | CUAUGUCGUAGUGGGUAAAmGmCmUmCmCmCmUmCmUmUmCmGmGmAmG | |
| | mGmGmAmGmCAUCAAAGNNNNNNNNNNNNNNNNNmN*mN*mN | |
| 174 (v7) | mA*mC*mU*GGmCGmCmUUUUAmUmCUGAUUACUUUGmAmGAGCCmAmU | 13755 |
| | mCmAmCCAGCmGmAmCmUAUmGmUmCmGUAGUGGmGmUAmAmAmGmCmU | |
| | mCmCmCmUmCmUmUmCmGmGmAmGmGmGmAmGmCmAmUCAAAGNNNNNN | |
| | NNNNNNNNNNN*mN*mN*mN | |
| 174 (v8) | mA*mC*mU*GGCGCUUUUAUCUGAUUACUUUGAGAGCCmAmUmCmAmCC | 13756 |
| | AGCmGmAmCmUAUmGmUmCmGUAGUGGmGmUmAmAAmGmCmUmCmCmCm | |
| | UmCmUmUmCmGmGmAmGmGmGmAmGmCmAmUCAAAGNNNNNNNNNNNNN | |
| | NNNN*mN*mN*mN | |
| 174 (v9) | mA*mC*mU*GGmCmGCmUUUUAmUmCUGAUUACUUUGmAmGAGCCAUCA | 13757 |
| | CCAGCmGmAmCmUAUmGmUmCmGUAGUGGGUAAAmGmCmUmCmCmCmUm | |
| | CmUmUmCmGmGmAmGmGmGmAmGmCAUCAAAGNNNNNNNNNNNNNNNNN | |
| | *mN*mN*mN | |
| 235 (v0) | ACUGGCGCUUCUAUCUGAUUACUCUGAGCGCCAUCACCAGCGACUAUGU | 13758 |
| | CGUAGUGGGUAAAGCCGCUUACGGACUUCGGUCCGUAAGAGGCAUCAGA | |
| | GNNNNNNNNNNNNNNNNNNNN | |
| 235 (v1) | mA*mC*mU*GGCGCUUCUAUCUGAUUACUCUGAGCGCCAUCACCAGCGA | 13759 |
| | CUAUGUCGUAGUGGGUAAAGCCGCUUACGGACUUCGGUCCGUAAGAGGC | |
| | AUCAGAGNNNNNNNNNNNNNNNNNmN*mN*mN | |
| 235 (v2) | mA*mC*mU*GGCGCUUCUAUCUGAUUACUCUGAGCGCCAUCACCAGCGA | 13760 |
| | CUAUGUCGUAGUGGGUAAAGCCGCUUACGGACUUCGGUCCGUAAGAGGC | |
| | AUCAGAGNNNNNNNNNNNNNNNNNNNN*mU*mU*mU | |
| 235 (v3) | mA*mC*mU*mGmGmCmGmCmUmUmCmUmAmUmCmUmGmAmUUACUCUGm | 13761 |
| | AmGmCmGmCmCmAmUmCmAmCmCAGCGAmCmUAUmGmUmCmGUAGUGmG | |
| | mGmUmAmAmAmGmCmCmGmCmUmUmAmCmGmGmAmCmUmUmCmGmGmUm | |
| | CmCmGmUmAmAmGmAmGmGmCmAmUmCmAGAGNNNNNNNNNNNNNNNNN | |
| | mN*mN*mN | |
| 235 (v4) | mA*mC*mU*mGmGmCmGmCUUCUmAmUmCmUmGmAmUUACUCUGmAmGm | 13762 |
| | CmGmCmCmAmUmCmAmCmCAGCGAmCmUAUmGmUmCmGUAGUGmGmGmU | |
| | mAmAmAmGmCmCmGmCmUmUmAmCmGmGmAmCmUmUmCmGmGmUmCmCm | |
| | GmUmAmAmGmAmGmGmCmAmUCAGAGNNNNNNNNNNNNNNNNNmN*mN* | |
| | mN | |
| 235 (v5) | mA*mC*mU*GGCGCUUCUAUCUGAUUACUCUGAGCGCCAUCACCAGCGA | 13763 |
| | mCmUAUmGmUmCmGUAGUGGGUmAmAmAmGmCmCmGmCmUmUmAmCmGm | |
| | GmAmCmUmUmCmGmGmUmCmCmGmUmAmAmGmAmGmGmCAUCAGAGNNN | |
| | NNNNNNNNNNNNNNmN*mN*mN | |
| 235 (v6) | mA*mC*mU*GGCGCUUCUAUCUGAUUACUCUGAGCGCCAUCACCAGCGA | 13764 |
| | CUAUGUCGUAGUGGGUmAmAmAmGmCmCmGmCmUmUmAmCmGmGmAmCm | |
| | UmUmCmGmGmUmCmCmGmUmAmAmGmAmGmGmCAUCAGAGNNNNNNNNN | |
| | NNNNNNNNmN*mN*mN | |
| 235 (v7) | mA*mC*mU*GGmCGmCmUUCUAmUmCUGAUUACUCUGmAmGCGCCmAmU | 13765 |
| | mCmAmCCAGCmGmAmCmUAUmGmUmCmGUAGUGGmGmUmAmAAmGmCmC | |
| | mGmCmUmUmAmCmGmGmAmCmUmUmCmGmGmUmCmCmGmUmAmAmGmAm | |
| | GmGmCmAmUCAGAGNNNNNNNNNNNNNNNNN*mN*mN*mN | |
| 235 (v8) | mA*mC*mU*GGCGCUUCUAUCUGAUUACUCUGAGCGCCmAmUmCmAmCC | 13766 |
| | AGCmGmAmCmUAUmGmUmCmGUAGUGGmGmUmAmAAmGmCmCmGmCmUm | |
| | UmAmCmGmGmAmCmUmUmCmGmGmUmCmCmGmUmAmAmGmAmGmGmCmA | |
| | mUCAGAGNNNNNNNNNNNNNNNNN*mN*mN*mN | |
| 235 (v9) | mA*mC*mU*GGmCGmCmUUCUAmUmCUGAUUACUCUGmAmGCGCCAUCA | 13767 |
| | CCAGCmGmAmCmUAUmGmUmCmGUAGUGGGUAAAmGmCmCmGmCmUmUm | |
| | AmCmGmGmAmCmUmUmCmGmGmUmCmCmGmUmAmAmGmAmGmGmCAUCA | |
| | GAGNNNNNNNNNNNNNNNNN*mN*mN*mN | |
| 316 (v0) | ACUGGCGCUUCUAUCUGAUUACUCUGAGCGCCAUCACCAGCGACUAUGU | 13768 |
| | CGUAGUGGGUAAAGCUCCCUCUUCGGAGGGAGCAUCAGAGNNNNNNNNN | |
| | NNNNNNNNNNN | |
| 316 (v1) | mA*mC*mU*GGCGCUUCUAUCUGAUUACUCUGAGCGCCAUCACCAGCGA | 13769 |
| | CUAUGUCGUAGUGGGUAAAGCUCCCUCUUCGGAGGGAGCAUCAGAGNNN | |
| | NNNNNNNNNNNNNN*mN*mN*mN | |
| 316 (v2) | mA*mC*mU*GGCGCUUCUAUCUGAUUACUCUGAGCGCCAUCACCAGCGA | 13770 |
| | CUAUGUCGUAGUGGGUAAAGCUCCCUCUUCGGAGGGAGCAUCAGAGNNN | |
| | NNNNNNNNNNNNNNNNN*mU*mU*mU | |
| 316 (v3) | mA*mC*mU*mGmGmCmGmCmUmUmCmUmAmUmCmUmGmAmUUACUCUGm | 13771 |
| | AmGmCmGmCmCmAmUmCmAmCmCAGCGAmCmUAUmGmUmCmGUAGUGmG | |
| | mGmUmAmAmAmGmCmUmCmCmCmUmCmUmUmCmGmGmAmGmGmGmAmGm | |
| | CmAmUmCmAGAGNNNNNNNNNNNNNNNNN*mN*mN*mN | |
| 316 (v4) | mA*mC*mU*mGmGmCmGmCUUCUmAmUmCmUmGmAmUUACUCUGmAmGm | 13772 |
| | CmGmCmCmAmUmCmAmCmCAGCGAmCmUAUmGmUmCmGUAGUGmGmGmU | |
| | mAmAmAmGmCmUmCmCmCmUmCmUmUmCmGmGmAmGmGmGmAmGmCmAm | |
| | UCAGAGNNNNNNNNNNNNNNNNN*mN*mN*mN | |
| 316 (v5) | mA*mC*mU*GGCGCUUCUAUCUGAUUACUCUGAGCGCCAUCACCAGCGA | 13773 |
| | mCmUAUmGmUmCmGUAGUGGGUAAAmGmCmUmCmCmCmUmCmUmUmCmG | |
| | mGmAmGmGmGmAmGmCAUCAGAGNNNNNNNNNNNNNNNNN*mN*mN*mN | |
| 316 (v6) | mA*mC*mU*GGCGCUUCUAUCUGAUUACUCUGAGCGCCAUCACCAGCGA | 13774 |
| | CUAUGUCGUAGUGGGUAAAmGmCmUmCmCmCmUmCmUmUmCmGmGmAmG | |
| | mGmGmAmGmCAUCAGAGNNNNNNNNNNNNNNNNN*mN*mN*mN | |
| 316 (v7) | mA*mC*mU*GGmCGmCmUUCUAmUmCUGAUUACUCUGmAmGCGCCmAmU | 13775 |
| | mCmAmCCAGCmGmAmCmUAUmGmUmCmGUAGUGGmGmUmAmAAmGmCmU | |
| | mCmCmCmUmCmUmUmCmGmGmAmGmGmGmAmGmCmAmUCAGAGNNNNNN | |
| | NNNNNNNNNNN*mN*mN*mN | |
| 316 (v8) | mA*mC*mU*GGCGCUUCUAUCUGAUUACUCUGAGCGCCmAmUmCmAmCC | 13776 |
| | AGCmGmAmCmUAUmGmUmCmGUAGUGGmGmUmAmAAmGmCmUmCmCmCm | |
| | UmCmUmUmCmGmGmAmGmGmGmAmGmCmAmUCAGAGNNNNNNNNNNNNN | |
| | NNNN*mN*mN*mN | |
| 316 (v9) | mA*mC*mU*GGmCGmCmUUCUAmUmCUGAUUACUCUGmAmGCGCCAUCA | 13777 |
| | CCAGCmGmAmCmUAUmGmUmCmGUAGUGGGUAAAmGmCmUmCmCmCmUm | |
| | CmUmUmCmGmGmAmGmGmGmAmGmCAUCAGAGNNNNNNNNNNNNNNNNN | |
| | *mN*mN*mN | |

TABLE 17
Sequences of gRNAs with their different chemical modification profiles (denoted by
version number) assayed in this example. Chemical modifications: * = phosphorothioate
bond; m = 2′OMe modification

| gRNA ID | | | |
|---|---|---|---|
| (scaffold | | | SEQ ID |
| variant-spacer) | Target | gRNA sequence | NO: |
| 174-6.7 | human | ACUGGCGCUUUUAUCUGAUUACUUUGAGAGCCAUCACCAGCGACUAU | 13994 |
| (v0) | PCSK9 | GUCGUAGUGGGUAAAGCUCCCUCUUCGGAGGGAGCAUCAAAGUCCUG | |
| | | GCUUCCUGGUGAAGA | |
| 174-6.7 | human | mA*mC*mU*GGCGCUUUUAUCUGAUUACUUUGAGAGCCAUCACCAGC | 13995 |
| (v1) | PCSK9 | GACUAUGUCGUAGUGGGUAAAGCUCCCUCUUCGGAGGGAGCAUCAAA | |
| | | GUCCUGGCUUCCUGGUGAmA*mG*mA | |
| 174-6.8 | human | ACUGGCGCUUUUAUCUGAUUACUUUGAGAGCCAUCACCAGCGACUAU | 13996 |
| (v0) | PCSK9 | GUCGUAGUGGGUAAAGCUCCCUCUUCGGAGGGAGCAUCAAAGUGGCU | |
| | | UCCUGGUGAAGAUGA | |
| 174-6.8 | human | mA*mC*mU*GGCGCUUUUAUCUGAUUACUUUGAGAGCCAUCACCAGC | 13997 |
| (v1) | PCSK9 | GACUAUGUCGUAGUGGGUAAAGCUCCCUCUUCGGAGGGAGCAUCAAA | |
| | | GUGGCUUCCUGGUGAAGAmU*mG*mA | |
| 174-7.9 | human | ACUGGCGCUUUUAUCUGAUUACUUUGAGAGCCAUCACCAGCGACUAU | 13998 |
| (v0) | B2M | GUCGUAGUGGGUAAAGCUCCCUCUUCGGAGGGAGCAUCAAAGGUGUA | |
| | | GUACAAGAGAUAGAA | |
| 174-7.9 | human | mA*mC*mU*GGCGCUUUUAUCUGAUUACUUUGAGAGCCAUCACCAGC | 13999 |
| (v1) | B2M | GACUAUGUCGUAGUGGGUAAAGCUCCCUCUUCGGAGGGAGCAUCAAA | |
| | | GGUGUAGUACAAGAGAUAmG*mA*mA | |
| 316-6.7 | human | ACUGGCGCUUCUAUCUGAUUACUCUGAGCGCCAUCACCAGCGACUAU | 14000 |
| (v0) | PCSK9 | GUCGUAGUGGGUAAAGCUCCCUCUUCGGAGGGAGCAUCAGAGUCCUG | |
| | | GCUUCCUGGUGAAGA | |
| 316-6.7 | human | mA*mC*mU*GGCGCUUCUAUCUGAUUACUCUGAGCGCCAUCACCAGC | 14001 |
| (v1′) | PCSK9 | GACUAUGUCGUAGUGGGUAAAGCUCCCUCUUCGGAGGGAGCAUCAGA | |
| | | GUCCUGGCUUCCUGGUGAmA*mG*mA | |
| 316-6.8 | human | ACUGGCGCUUCUAUCUGAUUACUCUGAGCGCCAUCACCAGCGACUAU | 14002 |
| (v0) | PCSK9 | GUCGUAGUGGGUAAAGCUCCCUCUUCGGAGGGAGCAUCAGAGUGGCU | |
| | | UCCUGGUGAAGAUGA | |
| 316-6.8 | human | mA*mC*mU*GGCGCUUCUAUCUGAUUACUCUGAGCGCCAUCACCAGC | 14003 |
| (v1′) | PCSK9 | GACUAUGUCGUAGUGGGUAAAGCUCCCUCUUCGGAGGGAGCAUCAGA | |
| | | GUGGCUUCCUGGUGAAGAmU*mG*mA | |
| 316-7.9 | human | ACUGGCGCUUCUAUCUGAUUACUCUGAGCGCCAUCACCAGCGACUAU | 14004 |
| (v0) | B2M | GUCGUAGUGGGUAAAGCUCCCUCUUCGGAGGGAGCAUCAGAGGUGUA | |
| | | GUACAAGAGAUAGAA | |
| 316-7.9 | human | mA*mC*mU*GGCGCUUCUAUCUGAUUACUCUGAGCGCCAUCACCAGC | 14005 |
| (v1′) | B2M | GACUAUGUCGUAGUGGGUAAAGCUCCCUCUUCGGAGGGAGCAUCAGA | |
| | | GGUGUAGUACAAGAGAUAmG*mA*mA | |
| 174-7.37 | human | ACUGGCGCUUUUAUCUgAUUACUUUGAGAGCCAUCACCAGCGACUAU | 14006 |
| (v0) | B2M | GUCGUAgUGGGUAAAGCUCCCUCUUCGGAGGGAGCAUCAAAGGGCCG | |
| | | AGAUGUCUCGCUC | |
| 174-7.37 | human | mA*mC*mU*GGCGCUUUUAUCUgAUUACUUUGAGAGCCAUCACCAGC | 14007 |
| (v1*) | B2M | GACUAUGUCGUAgUGGGUAAAGCUCCCUCUUCGGAGGGAGCAUCAAA | |
| | | GGGCCGAGAUGUCUCG*mC*mU*mC | |
| 235-6.7 | human | ACUGGCGCUUCUAUCUGAUUACUCUGAGCGCCAUCACCAGCGACUAU | 14008 |
| (v0) | PCSK9 | GUCGUAGUGGGUAAAGCCGCUUACGGACUUCGGUCCGUAAGAGGCAU | |
| | | CAGAGUCCUGGCUUCCUGGUGAAGA | |
| 235-6.7 | human | mA*mC*mU*GGCGCUUCUAUCUGAUUACUCUGAGCGCCAUCACCAGC | 14009 |
| (v1) | PCSK9 | GACUAUGUCGUAGUGGGUAAAGCCGCUUACGGACUUCGGUCCGUAAG | |
| | | AGGCAUCAGAGUCCUGGCUUCCUGGUGAmA*mG*mA | |
| 235-6.7 | human | mA*mC*mU*GGCGCUUCUAUCUGAUUACUCUGAGCGCCAUCACCAGC | 14010 |
| (v2) | PCSK9 | GACUAUGUCGUAGUGGGUAAAGCCGCUUACGGACUUCGGUCCGUAAG | |
| | | AGGCAUCAGAGUCCUGGCUUCCUGGUGAAGAU*mU*mU*mU | |
| 235-6.7 | human | mA*mC*mU*mGmGmCmGmCmUmUmCmUmAmUmCmUmGmAmUUACUCU | 14011 |
| (v3) | PCSK9 | GmAmGmCmGmCmCmAmUmCmAmCmCAGCGAmCmUAUmGmUmCmGUAG | |
| | | UGmGmGmUmAmAmAmGmCmCmGmCmUmUmAmCmGmGmAmCmUmUmCm | |
| | | GmGmUmCmCmGmUmAmAmGmAmGmGmCmAmUmCmAGAGUCCUGGCUU | |
| | | CCUGGUGAmA*mG*mA | |
| 235-6.7 | human | mA*mC*mU*mGmGmCmGmCUUCUmAmUmCmUmGmAmUUACUCUGmAm | 14012 |
| (v4) | PCSK9 | GmCmGmCmCmAmUmCmAmCmCAGCGAmCmUAUmGmUmCmGUAGUGmG | |
| | | mGmUmAmAmAmGmCmCmGmCmUmUmAmCmGmGmAmCmUmUmCmGmGm | |
| | | UmCmCmGmUmAmAmGmAmGmGmCmAmUCAGAGUCCUGGCUUCCUGGU | |
| | | GAmA*mG*mA | |
| 235-6.7 | human | mA*mC*mU*GGCGCUUCUAUCUGAUUACUCUGAGCGCCAUCACCAGC | 14013 |
| (v5) | PCSK9 | GAmCmUAUmGmUmCmGUAGUGGGUmAmAmAmGmCmCmGmCmUmUmAm | |
| | | CmGmGmAmCmUmUmCmGmGmUmCmCmGmUmAmAmGmAmGmGmCAUCA | |
| | | GAGUCCUGGCUUCCUGGUGAmA*mG*mA | |
| 235-6.7 | human | mA*mC*mU*GGCGCUUCUAUCUGAUUACUCUGAGCGCCAUCACCAGC | 14014 |
| (v6) | PCSK9 | GACUAUGUCGUAGUGGGUmAmAmAmGmCmCmGmCmUmUmAmCmGmGm | |
| | | AmCmUmUmCmGmGmUmCmCmGmUmAmAmGmAmGmGmCAUCAGAGUCC | |
| | | UGGCUUCCUGGUGAmA*mG*mA | |
| 235-6.8 | human | ACUGGCGCUUCUAUCUGAUUACUCUGAGCGCCAUCACCAGCGACUAU | 14015 |
| (v0) | PCSK9 | GUCGUAGUGGGUAAAGCCGCUUACGGACUUCGGUCCGUAAGAGGCAU | |
| | | CAGAGUGGCUUCCUGGUGAAGAUGA | |
| 235-6.8 | human | mA*mC*mU*GGCGCUUCUAUCUGAUUACUCUGAGCGCCAUCACCAGC | 14016 |
| (v1) | PCSK9 | GACUAUGUCGUAGUGGGUAAAGCCGCUUACGGACUUCGGUCCGUAAG | |
| | | AGGCAUCAGAGUGGCUUCCUGGUGAAGAmU*mG*mA | |
| 235-6.8 | human | mA*mC*mU*GGCGCUUCUAUCUGAUUACUCUGAGCGCCAUCACCAGC | 14017 |
| (v2) | PCSK9 | GACUAUGUCGUAGUGGGUAAAGCCGCUUACGGACUUCGGUCCGUAAG | |
| | | AGGCAUCAGAGUGGCUUCCUGGUGAAGAUGA*mU*mU*mU | |
| 235-6.8 | human | mA*mC*mU*mGmGmCmGmCmUmUmCmUmAmUmCmUmGmAmUUACUCU | 14018 |
| (v3) | PCSK9 | GmAmGmCmGmCmCmAmUmCmAmCmCAGCGAmCmUAUmGmUmCmGUAG | |
| | | UGmGmGmUmAmAmAmGmCmCmGmCmUmUmAmCmGmGmAmCmUmUmCm | |
| | | GmGmUmCmCmGmUmAmAmGmAmGmGmCmAmUmCmAGAGUGGCUUCCU | |
| | | GGUGAAGAmU*mG*mA | |
| 235-6.8 | human | mA*mC*mU*mGmGmCmGmCUUCUmAmUmCmUmGmAmUUACUCUGmAm | 14019 |
| (v4) | PCSK9 | GmCmGmCmCmAmUmCmAmCmCAGCGAmCmUAUmGmUmCmGUAGUGmG | |
| | | mGmUmAmAmAmGmCmCmGmCmUmUmAmCmGmGmAmCmUmUmCmGmGm | |
| | | UmCmCmGmUmAmAmGmAmGmGmCmAmUCAGAGUGGCUUCCUGGUGAA | |
| | | GAmU*mG*mA | |
| 235-6.8 | human | mA*mC*mU*GGCGCUUCUAUCUGAUUACUCUGAGCGCCAUCACCAGC | 14020 |
| (v5) | PCSK9 | GAmCmUAUmGmUmCmGUAGUGGGUmAmAmAmGmCmCmGmCmUmUmAm | |
| | | CmGmGmAmCmUmUmCmGmGmUmCmCmGmUmAmAmGmAmGmGmCAUCA | |
| | | GAGUGGCUUCCUGGUGAAGAmU*mG*mA | |
| 235-6.8 | human | mA*mC*mU*GGCGCUUCUAUCUGAUUACUCUGAGCGCCAUCACCAGC | 14021 |
| (v6) | PCSK9 | GACUAUGUCGUAGUGGGUmAmAmAmGmCmCmGmCmUmUmAmCmGmGm | |
| | | AmCmUmUmCmGmGmUmCmCmGmUmAmAmGmAmGmGmCAUCAGAGUGG | |
| | | CUUCCUGGUGAAGAmU*mG*mA | |
| 316-27.107 | mouse | ACUGGCGCUUCUAUCUGAUUACUCUGAGCGCCAUCACCAGCGACUAU | 14022 |
| (v0) | PCSK9 | GUCGUAGUGGGUAAAGCUCCCUCUUCGGAGGGAGCAUCAGAGCUGGC | |
| | | UUCUUGGUGAAGAUG | |
| 316-27.107 | mouse | mA*mC*mU*GGCGCUUCUAUCUGAUUACUCUGAGCGCCAUCACCAGC | 14023 |
| (v1) | PCSK9 | GACUAUGUCGUAGUGGGUAAAGCUCCCUCUUCGGAGGGAGCAUCAGA | |
| | | GCUGGCUUCUUGGUGAAG*mA*mU*mG | |
| 316-27.107 | mouse | mA*mC*mU*GGmCGmCmUUCUAmUmCUGAUUACUCUGmAmGCGCCmA | 14024 |
| (v7) | PCSK9 | mUmCmAmCCAGCmGmAmCmUAUmGmUmCmGUAGUGGmGmUmAmAAmG | |
| | | mCmUmCmCmCmUmCmUmUmCmGmGmAmGmGmGmAmGmCmAmUCAGAG | |
| | | CUGGCUUCUUGGUGAAG*mA*mU*mG | |
| 316-27.107 | mouse | mA*mC*mU*GGCGCUUCUAUCUGAUUACUCUGAGCGCCmAmUmCmAm | 14025 |
| (v8) | PCSK9 | CCAGCmGmAmCmUAUmGmUmCmGUAGUGGmGmUmAmAAmGmCmUmCm | |
| | | CmCmUmCmUmUmCmGmGmAmGmGmGmAmGmCmAmUCAGAGCUGGCUU | |
| | | CUUGGUGAAG*mA*mU*mG | |
| 316-27.107 | mouse | mA*mC*mU*GGmCGmCmUUCUAmUmCUGAUUACUCUGmAmGCGCCAU | 14026 |
| (v9*) | PCSK9 | CACCAGCmGmAmCmUAUmGmUmCmGUAGUGGGUAAAmGmCmUmCmCm | |
| | | CmUmCmUmUmCmGmGmAmGmGmGmAmGmCAUCAGAGCUGGCUUCUUG | |
| | | GUGAA*mG*mA*mU*mG | |
| 174-35.2 (v0) | ROSA26 | ACUGGCGCUUUUAUCUGAUUACUUUGAGAGCCAUCACCAGCGACUAU | 14027 |
| | | GUCGUAGUGGGUAAAGCUCCCUCUUCGGAGGGAGCAUCAAAGAGAAG | |
| | | AUGGGCGGGAGUCUU | |
| 174-35.2 (v2) | ROSA26 | mA*mC*mU*GGCGCUUUUAUCUGAUUACUUUGAGAGCCAUCACCAGC | 14028 |
| | | GACUAUGUCGUAGUGGGUAAAGCUCCCUCUUCGGAGGGAGCAUCAAA | |
| | | GAGAAGAUGGGCGGGAGUCUU*mU*mU*mU | |
| 316-35.2 (v0) | ROSA26 | ACUGGCGCUUCUAUCUGAUUACUCUGAGCGCCAUCACCAGCGACUAU | 14029 |
| | | GUCGUAGUGGGUAAAGCUCCCUCUUCGGAGGGAGCAUCAGAGAGAAG | |
| | | AUGGGCGGGAGUCUU | |
| 316-35.2 (v1) | ROSA26 | mA*mC*mU*GGCGCUUCUAUCUGAUUACUCUGAGCGCCAUCACCAGC | 14030 |
| | | GACUAUGUCGUAGUGGGUAAAGCUCCCUCUUCGGAGGGAGCAUCAGA | |
| | | GAGAAGAUGGGCGGGAGU*mC*mU*mU | |
| 316-35.2 (v5) | ROSA26 | mA*mC*mU*GGCGCUUCUAUCUGAUUACUCUGAGCGCCAUCACCAGC | 14031 |
| | | GAmCmUAUmGmUmCmGUAGUGGGUAAAmGmCmUmCmCmCmUmCmUmU | |
| | | mCmGmGmAmGmGmGmAmGmCAUCAGAGAGAAGAUGGGCGGGAGU*mC | |
| | | *mU*mU | |

Note that gRNAs annotated with a v1′ design contain one less phosphorothioate bond on the 3′ end of the gRNA. gRNAs annotated with v1* contain one extra phosphorothioate bond on the 3′end of the gRNA. gRNAs annotated with a v9* contain an extra phosphorothioate bond on the 3′ end of the gRNA.
Biochemical Characterization of gRNA Activity:

Target DNA oligonucleotides with fluorescent moieties on the 5′ ends were purchased commercially (sequences listed in Table 18). Double-stranded DNA (dsDNA) targets were formed by mixing the oligos in a 1:1 ratio in 1× cleavage buffer (20 mM Tris HCl pH 7.5, 150 mM NaCl, 1 mM TCEP, 5% glycerol, 10 mM MgCl₂), following by heating to 95° C. for 10 minutes, and then allowing the solution to cool to room temperature. CasX ribonucleoproteins (RNPs) were reconstituted with CasX 491 and the indicated gRNAs at a final concentration of 1 μM with 1.2-fold excess of the indicated gRNA in 1× cleavage buffer. RNPs were allowed to form at 37° C. for 10 minutes.

The effects of various structural and chemical modifications to the gRNA scaffold on the cleavage rate of CasX 491 RNPs were determined. Cleavage reactions were prepared with final RNP concentrations of 200 nM and final target concentrations of 10 nM, and reactions were carried out at 16° C. and initiated by the addition of the labeled target DNA substrate (Table 18). Aliquots of reactions were taken at 0.25, 0.5, 1, 2, 5, and 10 minutes and quenched by adding an equal volume of 95% formamide with 20 mM EDTA. Samples were denatured at 95° C. for 10 minutes and resolved on a 10% urea-PAGE gel. Gels were imaged on a Typhoon™ laser-scanner platform and quantified using ImageQuant™ TL 8.2 image analysis software (Cytiva™). The apparent first-order rate constant of non-target strand cleavage (k_{cleave-}) was determined for each CasX:gRNA combination.

To determine the competent fraction formed by each gRNA, cleavage reactions were prepared with final RNP concentrations of 100 nM and final target concentrations of 100 nM. Reactions were carried out at 37° C. and initiated by the addition of the labeled target substrate (Table 18). Aliquots were taken at 0.5, 1, 2, 5, 10, and 30 minutes and quenched by adding an equal volume of 95% formamide with 25 mM EDTA. Samples were denatured by heating at 95° C. for 10 minutes and resolved on a 10% urea-PAGE gel. Gels were imaged and quantified as above. CasX was assumed to act as a single-turnover enzyme under the assayed conditions, as indicated by the observation that sub-stoichiometric amounts of enzyme would fail to cleave a greater-than-stoichiometric amount of target substrate even under extended time-scales, and instead would approach a plateau that scaled with the amount of enzyme present. Thus, the fraction of target substrate cleaved over long time-scales by an equimolar amount of RNP would be indicative of the fraction of RNP that was properly formed and active for cleavage. The cleavage traces were fitted with a biphasic rate model, as the cleavage reaction clearly deviated from monophasic under this concentration regime. The plateau of each fit was determined and reported as the active fraction for each RNP in Table 21.

TABLE 18
Sequences of target DNA substrate oligonucleotides with fluorescent moieties on the
5′ ends used for biochemical characterization of gRNA activity.
/700/ = IRDye700; /800/ = IRDye800

| DNA substrate | Sequence |
|---|---|
| 6.7/6.8 target top | /700/catgtcttccatggccttcttcctggcttcctggtgaagatgagtggcgacctg |
| strand | ctggag |
| (SEQ ID NO: | |
| 14032) | |
| 6.7/6.8 target | /800/ctccagcaggtcgccactcatcttcaccaggaagccaggaagaaggccatggaa |
| bottom strand | gacatg |
| (SEQ ID NO: | |
| 14033) | |

In Vitro Transcription of CasX mRNA:

DNA templates encoding for CasX 491 (see Table 19 for encoding sequences) used for in vitro transcription were generated by PCR using forward primers containing a T7 promoter, followed by agarose gel extraction of the appropriately sized DNA. 25 ng/μL final concentration of template DNA was used in each in vitro transcription reaction that was carried out following the manufacturer's recommended protocol with slight modifications. Following in vitro transcription reaction incubation for 2-3 hours at 37° C., which were carried out with CleanCap® AG and N1-methyl-pseudouridine, DNAse digestion of template DNA and column-based purification using the Zymo RNA miniprep kit were performed. The poly(A) tail was added using E. coli PolyA Polymerase following the manufacturer's protocol, followed by column-based purification as stated above. Poly(A) tailed in vitro transcribed RNA was eluted in RNAse free water, analyzed on an Agilent TapeStation for integrity, and flash frozen prior to storage at −80° C.

TABLE 19
Encoding sequences of the CasX mRNA
molecules assessed in this example*

| | | DNA SEQ |
|---|---|---|
| CasX 491 mRNA ID | Component (ID) | ID NO: |
| CasX 491 mRNA #1 | 5′UTR | 14034 |
| | START codon + c-MYC | 14035 |
| | NLS + linker | |
| | CasX 491 | 13981 |
| | Linker + c-MYC NLS | 14036 |
| | P2A mScarlet + STOP codon | 14037 |
| CasX 676 mRNA #2 | 5′UTR | 14038 |
| | START codon + c-MYC NLS | 14039 |
| | CasX 676 | 14040 |
| | c-MYC NLS + STOP codons | 14041 |
| | 3′UTR | 14042 |
| | XbaI restriction site (partial) | 14043 |
| | Poly(A) tail | 14044 |

*Components are listed in a 5′ to 3′ order within the constructs

In Vitro Delivery of gRNA and CasX mRNA Via Transfection:

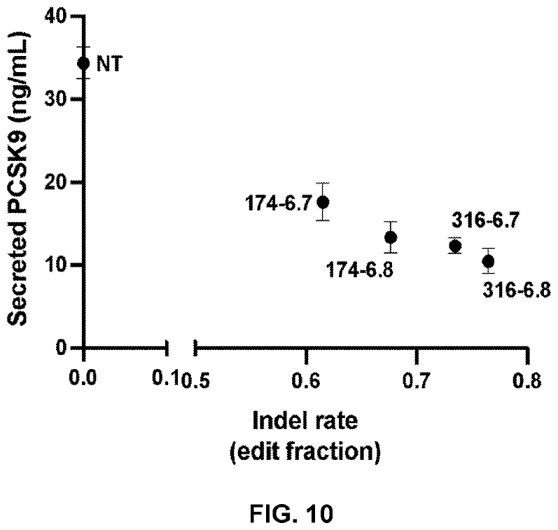

Editing at the PCSK9 locus and consequential effects on secreted PCSK9 levels were assessed for conditions using CasX 491 mRNA co-delivered with a PCSK9-targeting gRNA with scaffold variant 174 compared to conditions where a PCSK9-targeting gRNA with scaffold variant 316 was used. 100 ng of in vitro transcribed mRNA coding for CasX 491 with a P2A and mScarlet fluorescent protein was transfected into HepG2 cells with version 1 (v1) of gRNAs 174-6.7, 174-6.8, 316-6.7, and 316-6.8 (see Table 17) using lipofectamine. After a media change, the following were harvested at 28 hours post-transfection: 1) transfected cells were harvested for editing assessment at the PCSK9 locus by NGS; 2) media supernatant was harvested to measure secreted PCSK9 protein levels by ELISA. For editing analysis by NGS, amplicons were amplified from 200 ng of extracted gDNA with a set of primers targeting the PCSK9 locus and processed as described earlier in Example 4. Secreted PCSK9 levels in the media supernatant were also analyzed using a fluorescence resonance energy transfer-based immunoassay from CISBio following the manufacturer's instructions. Here, a gRNA using scaffold 174 with spacer 7.37 (v0; see Table 17), which targeted the endogenous B2M (beta-2-microglobulin) locus, served as the non-targeting (NT) control. These results are shown in FIG. 10.

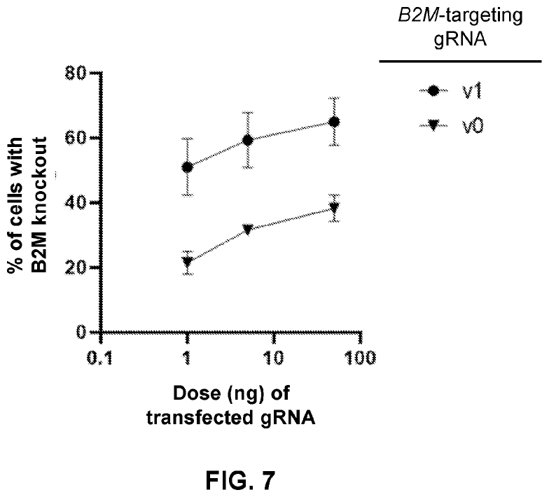

To compare the editing potency of version 0 (v0) and version 1 (v1) of B2M-targeting gRNAs, ~6E4 HepG2 hepatocytes were seeded per well of a 96-well plate. 24 hours later, seeded cells were co-transfected using lipofectamine with 100 ng of in vitro transcribed mRNA coding for CasX 491 and different doses (1, 5, or 50 ng) of either v0 or v1 of the B2M-targeting gRNA containing scaffold variant 174 and spacer 7.37 (see Table 17). Six days post-transfection, cells were harvested for B2M protein expression analysis via immunostaining of the B2M-dependent HLA protein, followed by flow cytometry using the Attune™ NxT flow cytometer. These results are shown in FIG. 7.

V1 through v6 variants of chemically-modified PCSK9-targeting gRNAs (Table 17) were assessed for their effects on editing potency and consequential effects on secreted PCSK9 levels in vitro. Briefly, 100 ng of in vitro transcribed mRNA coding for CasX variant 491, P2A and mScarlet fluorescent protein was transfected into HepG2 cells with 50 ng of the indicated chemically-modified gRNA using lipofectamine. After a media change, the following were harvested at 28 hours post-transfection: 1) transfected cells for editing assessment at the PCSK9 locus by NGS as described above; 2) media supernatant to measure secreted PCSK9 protein levels by ELISA, as described above. Here, a B2M-targeting gRNA was used as a non-targeting control. These results are shown in Table 22.

LNP (lipid nanoparticle) co-formulations were performed as described in Example 12.

Delivery of LNPs Encapsulating CasX mRNA and Targeting gRNAs In Vitro:

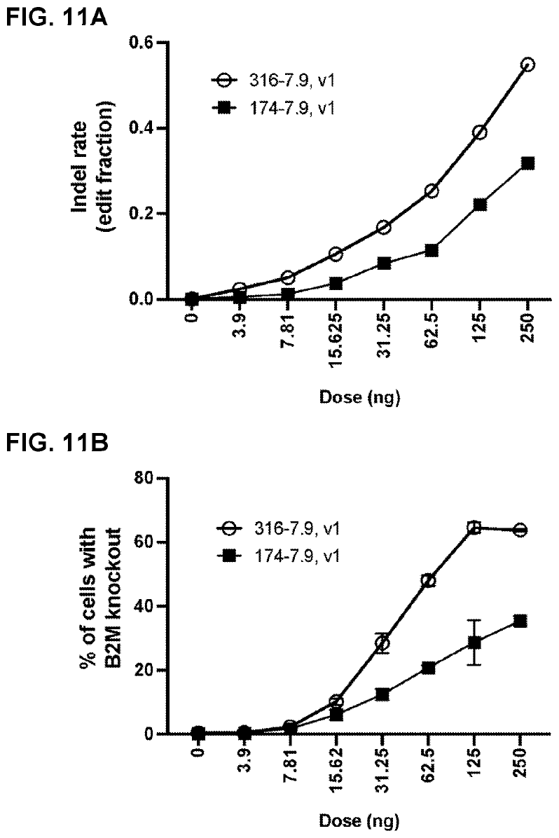

~50,000 HepG2 cells, cultured in DMEM/F-12 media containing 10% FBS and 1% PenStrep, were seeded per well in a 96-well plate. The next day, seeded cells were treated with varying concentrations of LNPs, which were prepared in six 2-fold serial dilutions starting at 250 ng. These LNPs were formulated to encapsulate CasX 491 mRNA and a B2M-targeting gRNA incorporating either scaffold variant 174 or 316 with spacer 7.9 (v1; see Table 17). Media was changed 24 hours after LNP treatment, and cells were cultured for six additional days prior to harvesting for gDNA extraction for editing assessment at the B2M locus by NGS and B2M protein expression analysis via HLA immunostaining, followed by flow cytometry using the Attune NxT flow cytometer. Briefly, for editing assessment, amplicons were amplified from 200 ng of extracted gDNA with primers targeting the human B2M locus and processed as described in Example 4. The results of these assays are shown in FIGS. 11A and 11B.

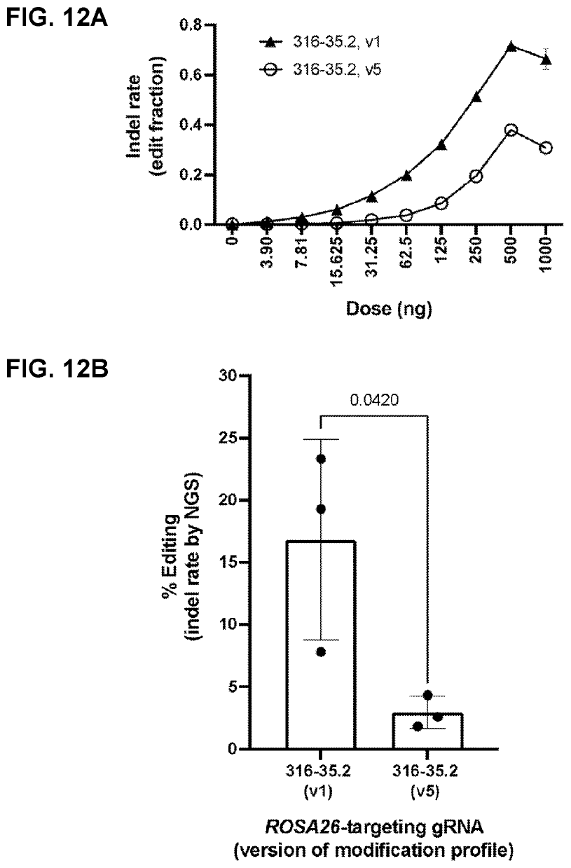

~20,000 mouse Hepa 1-6 hepatocytes were seeded per well in a 96-well plate. The following day, seeded cells were treated with varying concentrations of LNPs, which were prepared in eight 2-fold serial dilutions starting at 1000 ng. These LNPs were formulated to encapsulate CasX 676 mRNA #2 (see Table 19) and a ROSA26-targeting gRNA incorporating scaffold variant 316 with spacer 35.2 (v1 or 5; see Table 17). Media was changed 24 hours post-treatment with LNPs, and cells were cultured for seven additional days prior to harvesting for gDNA extraction for editing assessment at the ROSA26 locus by NGS. Briefly, amplicons were amplified from extracted gDNA with primers targeting the mouse ROSA26 locus and processed as described in Example 4. The results of this experiment are shown in FIG. 12A.

Delivery of LNPs Encapsulating CasX mRNA and Targeting gRNA In Vivo:

LNP co-formulations were performed as described in Example 12.

To assess the effects of using v1 and v5 of scaffold 316 in vivo, CasX 676 mRNA #2 (see Table 19) and a ROSA26-targeting gRNA using scaffold 316 with spacer 35.2 (v1 or v5; see Table 17) were encapsulated within the same LNP using a 1:1 mass ratio for mRNA:gRNA. Formulated LNPs were buffer-exchanged to PBS for in vivo injection. In vivo administration of formulated LNPs was performed as detailed in Example 9. Briefly, LNPs were administered retro-orbitally into 4-week old C57BL/6 mice. Six days post-administration, mice were euthanized, and the liver tissue was harvested for gDNA extraction using the Zymo Research Quick DNA/RNA Miniprep kit following the manufacturer's instructions. Target amplicons were then amplified from the extracted gDNA with a set of primers targeting the mouse ROSA26 locus and processed as described earlier in Example 4 for editing assessment by NGS. The results of this experiment are shown in FIG. 12B.

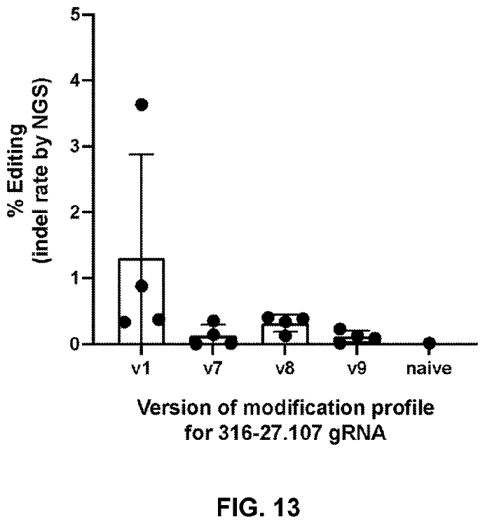

To compare the effects of using v7, v8, and v9 of scaffold 316 on editing at the PCSK9 locus in vivo, CasX 676 mRNA #1 (see Table 20 for sequences) and a PCSK9-targeting gRNA using scaffold 316 with spacer 27.107 (v1, v7, v8, or v9; see Table 17), were encapsulated within the same LNP using a 1:1 mass ratio for mRNA:gRNA for each gRNA. In vivo administration of formulated LNPs was performed as detailed in Example 9. Briefly, LNPs were administered retro-orbitally into 6-week old C57BL/6 mice, and mice were euthanized seven days post-injection to harvest liver tissue for gDNA extraction for editing assessment by NGS at the PCSK9 locus. The results of this experiment are shown in FIG. 13.

TABLE 20
Encoding sequences of CasX 676 mRNA #1 molecule

| | | | DNA SEQ |
|---|---|---|---|
| CasX ID | Component (ID) | Description | ID NO: |
| CasX 676 mRNA #1 | 5′UTR | hHBA | 14045 |
| | START codon + | | 14039 |
| | c-MYC NLS | | |
| | CasX 676 | | 14040 |
| | c-MYC NLS + | | 14041 |
| | STOP codons | | |
| | 3′UTR | hHBA | 14046 |
| | Poly(A) tail | | 14044 |

*Components are listed in a 5′ to 3′ order within the constructs

### Results

Assessing the Effects of Various Chemical Modifications on gRNA Activity:

Several studies involving Cas9 have demonstrated that chemical modifications of the gRNA resulted in significantly improved editing activity when delivered with Cas9 mRNA. Following delivery of Cas9 mRNA and gRNA into target cells, unprotected gRNA is susceptible to degradation during the mRNA translation process. Addition of chemical modifications such as 2′O-methyl(2′OMe) groups and phosphorothioate bonds can reduce the susceptibility of the gRNA to cellular RNases, but also have the potential to disrupt folding of the gRNA and its interactions with the CRISPR-Cas protein. Given the lack of structural similarity between CasX and Cas9, as well as their respective gRNAs, appropriate chemical modification profiles must be designed and validated de novo. Using published structures of wild-type CasX from Deltaproteobacteria (PDB codes 6NY1, 6NY2, and 6NY3) as reference, residues that appeared potentially amenable to modification were selected. However, the published structures were of a wild type CasX ortholog and gRNA distinct from the species used as the basis for the engineered variants presented here, and they also lacked the resolution to confidently determine interactions between protein side-chains and the RNA backbone. These limitations introduced a significant amount of ambiguity into determining which nucleotides might be safely modified. As a result, six profiles of chemical modifications (denoted as versions) were designed for initial testing, and these six profiles are illustrated in FIGS. 6A and 6B. The v1 profile was designed as a simple end-protected structure, where the first and last three nucleotides were modified with 2′OMe and phosphorothioate bonds. In the v2 profile, 3′UUU tail was added to mimic the termination sequence used in cellular transcription systems and to move the modified nucleotides outside of the region of the spacer involved in target recognition. The v3 profile included the end protection as in v1, as well as the addition of 2′OMe modifications at all nucleotides identified to be potentially modifiable based on structural analysis. The v4 profile was modeled based on v3, but with all the modifications in the triplex region removed, as this structure was predicted to be more sensitive to any perturbation of the RNA helical structure and backbone flexibility. The v5 profile maintained chemical modifications in the scaffold stem and extended stem regions, while the v6 profile harbored modifications only in the extended stem. The extended stem is a region that would become fully exposed to solvent in the RNP and is amenable to replacement by other hairpin structures, and therefore presumably relatively insensitive to chemical modifications.

The minimally modified v1 gRNA was initially assessed against an unmodified gRNA (v0) to determine the potential benefit of such chemical modifications on editing when the gRNA was co-delivered with CasX mRNA to target cells. Modified (v1) and unmodified (v0) B2M-targeting gRNAs with spacer 7.37 were co-transfected with CasX mRNA into HepG2 cells, and editing at the B2M locus was measured by loss of surface presentation of the B2M-dependent HLA complex, as detected by flow cytometry (FIG. 7). The data demonstrate that use of the v1 gRNA resulted in substantially greater loss of B2M expression compared to the levels seen with v0 gRNA across the various doses, thereby confirming that end modifications of the gRNA increased CasX-mediated editing activity upon delivery of the CasX mRNA and gRNA.

The broader set of gRNA chemical modification profiles were assessed using PCSK9-targeting gRNAs using scaffold variant 235 and spacers 6.7 and 6.8 to determine whether the additional chemical modifications would be able to support the formation of active RNPs. In vitro cleavage assays described above were performed to determine k_{cleave} and fraction competence for these engineered gRNAs harboring the various chemical modification profiles. The results from these in vitro cleavage assays are shown in Table 21. The data demonstrate that gRNAs with the v3 profiles exhibited no activity, an indication that the addition of some chemical modifications significantly interfered with RNP formation or activity. Adding v4 chemical modifications resulted in a reasonable cleavage rate in the excess RNP condition, but exhibited very low fraction competence. The difference between v3 and v4 modifications confirmed that modifications in the triplex region prevented the formation of any active RNP, either due to the inability of the gRNA to fold properly or a disruption in the gRNA-protein interactions. The reduced fraction competence resulting from appending v4 modifications suggest that while the gRNA was able to successfully assemble with the CasX protein to form a cleavage-competent RNP, a large majority of the gRNA was misfolded, or that the appended chemical modifications reduced the affinity of the gRNA for the CasX protein and impeded the efficiency of RNP formation. Application of the v5 or v6 profiles resulted in competent fractions that were comparable to, but slightly lower than, those obtained for reactions using the v1 and v2 modifications. While the k_{cleave} values were relatively consistent between v5 and v6 gRNAs, both v5 and v6 gRNAs achieved nearly half of the k_{cleave} values for v1 and v2 gRNAs. The reduced k_{cleave} value for v6 gRNA was particularly surprising, given the lack of expected interaction between the gRNA and CasX protein in the modified extended stem. However, for both v5 and v6 gRNAs, it is possible that the reduced flexibility of the gRNA, resulting from the 2′OMe modifications, inhibited structural changes in the RNP required for efficient cleavage, or that the modified initial base-pairs of the hairpin involved in CasX protein interaction had been negatively impacted by the inclusion of the 2′OMe groups.

TABLE 21
Parameters of cleavage activity assessed for CasX
RNPs with the various PCSK9-targeting gRNAs using
scaffold 235 and harboring the indicated chemical
modification profile, denoted by version number

| gRNA | | |
|---|---|---|
| (scaffold variant- | | |
| spacer, version no.) | K_{cleave} (min⁻¹) | Fraction competence |
| 235-6.7, v1 | 0.901 | 0.398 |
| 235-6.8, v1 | 1.36 | 0.398 |
| 235-6.7, v2 | 0.454 | 0.386 |
| 235-6.8, v2 | 2.03 | 0.361 |
| 235-6.7, v3 | 0 | 0 |
| 235-6.8, v3 | 0 | 0 |
| 235-6.7, v4 | 0.434 | 0.031 |
| 235-6.8, v4 | 0.257 | 0.005 |
| 235-6.7, v5 | 0.506 | 0.313 |
| 235-6.8, v5 | 0.680 | 0.388 |
| 235-6.7, v6 | 0.462 | 0.346 |
| 235-6.8, v6 | 0.715 | 0.325 |

The chemically-modified PCSK9-targeting gRNAs based on scaffold 235 were subsequently assessed for editing in a cell-based assay. CasX mRNA and chemically modified PCSK9-targeting gRNAs were co-transfected into HepG2 cells using lipofectamine. Editing levels were measured by indel rate at the PCSK9 locus by NGS and secreted PCSK9 levels by ELISA, and the data are displayed in Table 22. The data demonstrate that use of v3 and v4 gRNAs resulted in minimal editing activity at the PCSK9 locus, consistent with findings from the biochemical in vitro cleavage assays shown in Table 21. Meanwhile, use of v5 and v6 gRNAs resulted in editing levels, measured by indel rate and PCSK9 secretion, that were slightly lower than the levels attained with use of v1 and v2 gRNAs (Table 22). Specifically, the results show that use of v1 and v2 gRNAs, which harbored end modifications, resulted in ~80-85% editing at the PCSK9 locus, indicating that adding chemical modifications to the gRNA ends was sufficient to achieve efficient editing with CasX. While the data demonstrate that use of v5 and v6 gRNAs resulted in efficient editing in vitro, near-saturating levels of editing were observed with use of the v1 gRNA in this experiment where a single dose of the gRNA was transfected. As a result, the use of a single dose rendered it challenging to assess clearly the effects of the chemical modifications on editing under guide-limiting conditions. Therefore, profiles v1 and v5 were chosen for further testing, as v1 contains the simplest modification profile, and v5 is the most heavily modified profile whose application demonstrated robust activity in vitro (Tables 21 and 22).

TABLE 22
Editing levels measured by indel rate at PCSK9 locus by NGS and
secreted PCSK9 levels by ELISA in HepG2 cells co-transfected
with CasX 491 mRNA and various chemically-modified PCSK9-targeting
gRNAs using scaffold 235 and either spacer 6.7 or 6.8

| Experimental | Indel rate (edit fraction) | | Secreted PCSK9 (ng/mL) | |
|---|---|---|---|---|
| condition | Mean | Stdev | Mean | Stdev |
| CasX mRNA | 0.0021 | 0.003 | 52 | 14 |
| only | | | | |
| 235-6.7, v1 | 0.83 | 0.0058 | 18 | 5.7 |
| 235-6.7, v2 | 0.79 | 0.0071 | 21 | 4 |
| 235-6.7, v3 | 0.024 | 0.02 | 48 | 19 |
| 235-6.7, v4 | 0.12 | 0.006 | 34 | 5.5 |
| 235-6.7, v5 | 0.73 | 0.023 | 21 | 9 |
| 235-6.7, v6 | 0.75 | 0.0069 | 22 | 8.8 |
| 235-6.8, v1 | 0.85 | 0.017 | 16 | 4.4 |
| 235-6.8, v2 | 0.83 | 0.0028 | 20 | 1.5 |
| 235-6.8, v3 | 0.023 | 0.0027 | 39 | 2.7 |
| 235-6.8, v4 | 0.088 | 0.0086 | 42 | 10 |
| 235-6.8, v5 | 0.77 | 0.017 | 19 | 1.6 |
| 235-6.8, v6 | 0.78 | 0.014 | 24 | 6.9 |
| Non-targeting | 0.0019 | 0.0026 | 42 | 12 |
| ctrl | | | | |

The v1 and v5 profiles were further tested in another cell-based assay to assess their effects on editing efficiency. LNPs were formulated to co-encapsulate CasX mRNA #2 and v1 and v5 chemically-modified ROSA26-targeting gRNAs using the newly-designed gRNA scaffold 316 (described further in the following sub-section). The “v5” profile was modified slightly for application to the 316 scaffold. Three 2′ OMe modifications in the non-base-paired region immediately 5′ of the extended stem were removed to restrict modifications to the two stemloop regions. Hepa 1-6 hepatocytes were treated with the resulting LNPs at various doses and harvested eight days post-treatment to assess editing at the ROSA26 locus, measured as indel rate detected by NGS (FIG. 12A). The data demonstrate that treatment with LNPs delivering the v5 ROSA26-targeting gRNA resulted in markedly lower editing levels across the range of doses compared to the levels achieved with the v1 counterpart (FIG. 12A). There are several possible explanations for the differences in relative activity observed with use of v5 gRNA in FIG. 12A relative to that observed in Table 22. The first and most likely possible explanation is that the single dose used to achieve editing shown in Table 22 was too high to measure differences in activity accurately between use of v5 gRNA and v1 gRNA. It is also possible that the removal of the modifications outside the stemloop motifs in the 316 version of v5 negatively impacted guide activity. While it is possible that these modifications provide stability benefits that outweigh an activity cost imparted by the stemloop modifications, this seems unlikely given that increasing levels of modification have so far resulted in decreased activity. A final possible explanation is that the modifications in the v5 profile might negatively impact LNP formulation or behavior through differential interactions between the modified nucleotide backbone and the ionizable lipid of the LNP, potentially resulting in less efficient gRNA encapsulation or in less efficient gRNA release following internalization.

LNPs co-encapsulating the CasX mRNA #2 and v1 and v5 chemically-modified ROSA26-targeting gRNAs based on scaffold 316 were further tested in vivo. FIG. 12B shows the results of the editing assay as percent editing measured as indel rate at the ROSA26 locus. The data demonstrate that use of the v5 gRNA resulted in ~5-fold lower editing compared to that achieved with use of the v1 gRNA, under more relevant testing conditions of in vivo LNP delivery. These findings support the reduced cleavage rate observed biochemically for the v5 gRNA in Table 21, an indication that the v5 modifications have interfered with some aspect of CasX activity. Given the consistent decrease in activity detected in v5 and v6 profiles (Table 21), the reduced editing may be attributed to modifications in the extended stem region. Although the extended stem of the gRNA has minimal interactions with the CasX protein, it is possible that addition of 2′OMe groups at the first base-pair disrupted either the CasX protein-gRNA interactions or the complex RNA fold where the extended stem meets the pseudoknot and triplex regions. More specifically, inclusion of the 2′OMe groups might have adversely affected the basal base-pairs of the gRNA extended stem and residues R49, K50, and K51 of the CasX protein. Finally, structural studies of CasX have suggested that flexibility of the gRNA is required for efficient DNA cleavage (Liu J, et al, CasX enzymes comprise a distinct family of RNA-guided genome editors. Nature 566: 218-223 (2019); Tsuchida C A, et al, Chimeric CRISPR-CasX enzymes and guide RNAs for improved genome editing activity. Mol Cell 82(6): 1199-1209 (2022)). Thus, the addition of the 2′OMe groups throughout the extended stem might have enforced a more rigid A-form helical structure and prevented the needed flexibility for the gRNA for efficient cleavage. Furthermore, it is possible that the additional modifications in the scaffold stem in the v5 and v6 profiles might be detrimental to activity, though this is currently unclear given the limited comparisons between the v5 and v6 profiles.

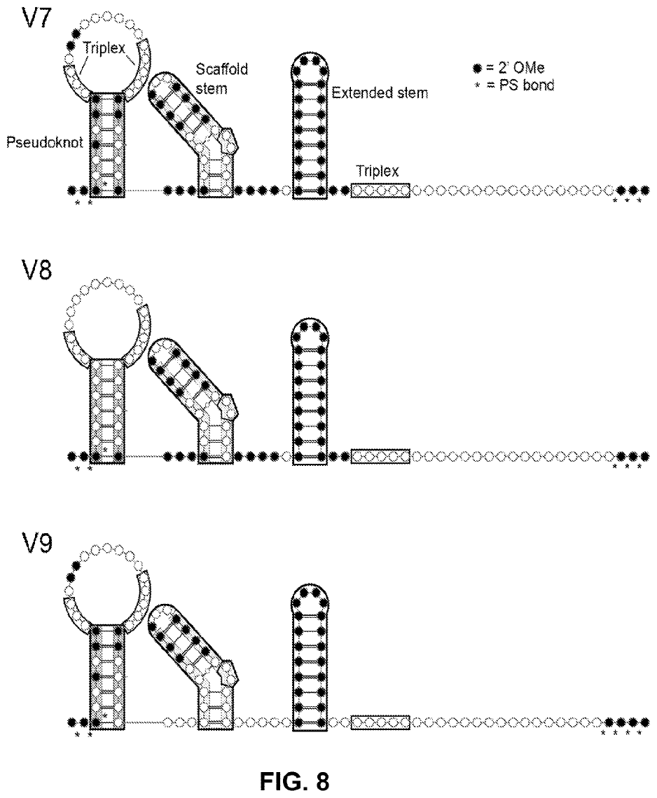

Additional modification profiles were designed with the goal of enhancing gRNA stability while mitigating the adverse effects on RNP cleavage activity. Using recently published structures of wild-type CasX from Planctomycetes (PDB codes 7WAY, 7WAZ, 7WB0, 7WB1), which has a higher homology to the engineered CasXs being assessed, additional chemical modification profiles for gRNAs were designed and are illustrated in FIG. 8. These profiles illustrate the addition of 2′OMe groups and phosphorothioate bonds to a newly-designed gRNA scaffold variant, which is described in the ensuing sub-section. These new gRNA chemical modification profiles were designed based on the initial data demonstrating sufficient editing activity observed in Table 22 with use of the v5 gRNA that suggested that modifications to the extended stem and scaffold stem regions would not negatively impact activity. The v7 profile was designed to include 2′OMe at residues likely to be modifiable throughout the gRNA structure, which excluded the triplex region, given the dramatic negative effects of adding such modifications observed earlier with the v3 profile. More conservative profiles, v8 and v9, were also designed, as illustrated in FIG. 8. For the v8 construct, modifications were removed in the pseudoknot and triplex loop region, but were retained in the scaffold stem, extended stem, and their flanking single-stranded regions, in addition to the 5′ and 3′ termini. For the v9 profile, modifications were removed in the single-stranded regions flanking the stemloops, but were retained in the stemloops themselves, in addition to the pseudoknot, triplex loop, and 5′ and 3′ termini. The additional chemical modification profiles v7, v8, and v9 of the newly designed gRNA scaffold variant 316 (discussed further below) were assessed in vivo at the PCSK9 locus. The results of the editing assay in vivo quantified as percent editing at the PCSK9 locus measured as indel rate as detected NGS are illustrated in FIG. 13. Despite the fact that low editing efficiency was detected overall, the data demonstrate that use of v7, v8, and v9 gRNAs resulted in lower editing levels at the PCSK9 locus compared to the indel rate achieved with use of the v1 gRNA (FIG. 13). Given the findings in FIGS. 12A-12B showing inferior editing activity attained with the v5 gRNA, it is unsurprising that v7, v8, and v9 profiles similarly demonstrated comparatively lower editing activity. As illustrated in FIG. 8, the v7, v8, and v9 profiles include modifications throughout the extended stem region, which might have interfered with RNP activity.

Comparison of gRNA Scaffold Variant 174 and 316 Using an In Vitro Cleavage Assay:

Previous work had established gRNA scaffold variant 235 as the top-performing scaffold variant across multiple delivery conditions. However, the longer length of scaffold 235 (119 bp, when using a 20 bp spacer) relative to gRNAs including scaffold 174 (109 bp, when using a 20 bp spacer) increased the difficulty of solid-phase RNA synthesis, which would result in increased manufacturing costs, decreased purity and yield, and higher rates of synthesis failures. To address these issues but retain the improved activity of using scaffold variant 235, a chimeric gRNA scaffold was designed primarily on the basis of the scaffold 235 sequence, but the extended stemloop of scaffold 235 was replaced with the shorter extended stemloop of scaffold variant 174 (FIGS. 9A-9C). The resulting chimeric scaffold, named scaffold 316, was synthesized in parallel with scaffold 174 and PCSK9-targeting spacers 6.7 and 6.8, and B2M-targeting spacer 7.9 harboring the v1 chemical modification profile, with 2′OMe and phosphorothioate bonds on the first and last three nucleotides of all gRNAs (see Table 17). Scaffold variant 174 was chosen as the comparator rather than variant 235 because variant 174 was the best previously characterized scaffold with the same length as variant 316.

In vitro cleavage activity was assessed for gRNAs with scaffold 174 and 316 and spacers 6.7 and 6.8. Cleavage assays were carried out with 20-fold excess RNP over a matching dsDNA target. Cleavage rates were quantified for all four guides, and the results are shown in Table 23. The data demonstrate that in the context of spacer 6.7, use of either scaffold 174 or 316 resulted in similar cleavage rates, with scaffold 316 resulting in marginally faster cleavage than that achieved with scaffold 174. In the context of spacer 6.8, the difference in cleavage activity was more pronounced: CasX RNPs using scaffold 316 were able to cleave DNA nearly twice as quickly as CasX RNPs using scaffold 174 (Table 23).

Assays were also performed with equimolar amounts of RNP and DNA target over a longer time course to assess the fraction of expected RNP active for cleavage. As the CasX RNP is essentially single-turnover over the tested timescale, and the concentrations used are expected to be substantially higher than the K_{D} of the DNA-binding reaction, the amount of cleaved DNA should approximate the amount of active RNP. For either spacer 6.7 or 6.8, the active fraction of CasX RNPs incorporating scaffold 316 was 25-30% higher than for CasX RNPs using scaffold 174 (Table 23). These data suggest that a higher fraction of gRNA using scaffold 316 was properly folded for association with the CasX protein, or that the gRNA using scaffold 316 was able to associate more strongly with the CasX protein. Compared to scaffold 174, scaffold 316 harbors mutations expected to stabilize the pseudoknot and triplex structures required for proper gRNA folding. The increased stability of these motifs in particular, which were more likely to misfold than the simple hairpins found elsewhere in the gRNA structure, might result in a slightly higher fraction of the gRNAs folding into an active conformation.

TABLE 23
Parameters of cleavage activity assessed for CasX RNPs
with gRNAs containing scaffold variant 174 or 316 with
the version 1 (v1) chemical modification profile

| gRNA | | |
|---|---|---|
| (scaffold variant-spacer) | k_{cleave} (min⁻¹) | Fraction competence |
| 174-6.7, v1 | 0.236 | 0.194 |
| 174-6.8, v1 | 0.142 | 0.165 |
| 316-6.7, v1 | 0.264 | 0.244 |
| 316-6.8, v1 | 0.272 | 0.213 |

Comparison of gRNA Scaffold Variant 174 and 316 in a Cell-Based Assay:

An editing assessment using gRNA scaffold variant 174 compared to variant 316 was performed in a cell-based assay. CasX 491 mRNA and the version 1 (v1) of PCSK9-targeting gRNAs using spacers 6.7 and 6.8 were lipofected into HepG2 cells. Treated cells were harvested 28 hours post-transfection for analysis of editing levels at the PCSK9 locus by NGS and secreted PCSK9 levels by ELISA, and the data are presented in FIG. 10. The data demonstrate that use of any of the PCSK9-targeting gRNA tested resulted in efficient editing at the PCSK9 locus and substantial reduction in PCSK9 secretion compared to the non-targeting control using the B2M-targeting gRNA. The results also show that use of scaffold 316 resulted in more effective editing at the PCSK9 locus than that observed with use of scaffold 174 (~10 percentage point increase in editing rate achieved with scaffold 316 over scaffold 174). This finding is further supported by the ELISA results, such that use of scaffold 316 resulted in more effective reduction of PCSK9 secretion compared to that achieved with use of scaffold 174.

Scaffold variants 174 and 316 were also assessed in an editing assay where LNPs were formulated to co-encapsulate CasX 491 mRNA and B2M-targeting gRNA harboring either scaffold variant. HepG2 cells were treated with the resulting LNPs at various doses and harvested seven days post-treatment to assess editing at the B2M locus, measured as indel rate detected by NGS (FIG. 11A) and loss of surface presentation of the B2M-dependent HLA complex, as detected by flow cytometry (FIG. 11B). The results from both assays demonstrate that treatment with LNPs to deliver the B2M-targeting gRNA using scaffold 316 resulted in higher editing potency at the B2M locus compared to LNPs delivering the gRNA using scaffold 174 at each dose (FIGS. 11A and 11B). Specifically, at the highest dose of 250 ng, use of scaffold 316 resulted in an editing level that was nearly two-fold higher than the level attained with using scaffold 174. This substantial increase in editing efficacy when using scaffold 316 versus scaffold 174, compared to the comparatively modest difference in activity observed from the in vitro cleavage assays, might be attributed to the destabilization of gRNA structure and folding during LNP formulation. The low pH conditions and association of cationic lipids during LNP formulation could adversely affect parts of the gRNA structure and result in unfolding. Consequently, it would be necessary for the gRNA to refold quickly in the cytoplasm upon delivery, both to bind the CasX protein to form the RNP and to evade RNase degradation. The stability-increasing mutations in scaffold 316 compared to scaffold 174 might provide a substantial benefit in supporting proper gRNA refolding in the cytoplasm after LNP delivery, while the deliberate folding protocol carried out for the gRNA prior to biochemical experiments likely reduced the impact of these mutations.

Example 7: Design and Assessment of Codon-Optimized CasX mRNA on Editing Efficiency when Delivered Together with Targeting gRNAs In Vitro

mRNA sequence and associated modifications can have a significant impact on the efficacy of mRNA-based delivery. Modified nucleotides, including those that encode the 5′ cap structure, are important determinants of mRNA stability, translatability, and immunogenicity. Here, for all designed and tested CasX mRNAs, a “Cap 1” structure was used, which included a 5′ m7G in a 5′-5′ triphosphate linkage to an initiating nucleotide with a 2′OMe modification. This structure, similar to the “Cap 0” structure lacking the 2′OMe modification, promotes efficient translation, and has reduced immunogenicity compared to the “Cap 0” structure. Furthermore, the use of modified nucleobases can reduce immunogenicity of the mRNA. Here, the N1-methyl-pseudouridine was used to substitute the uridine ribonucleoside for all in vitro transcription reactions, since published studies have demonstrated that the N1-methyl-pseudouridine substantially enhances mRNA performance and reduces mRNA immunogenicity. The modifications are expected to result in reduced immunogenicity and higher translation rates in vivo, potentially by avoiding activation of RIG-I, a primary cytosolic sensor for double-stranded RNA, which is a common contaminant of in vitro transcribed mRNA.

Optimization of the poly(A) tail will also be explored. The poly(A) tail is required for translation and mRNA stability, with longer tails being associated with a longer mRNA half-life. Polyadenylation can be carried out post-transcriptionally with a poly(A) polymerase, but this results in variable tail lengths and adds a step to the mRNA production process. mRNA productions were conducted using plasmids containing a template 80A-tail, terminating with a Type IIS restriction site to allow for run-off transcription, as constructs with plasmids containing a template 120A-tail were unstable during propagation in E. coli, often resulting in clones with significant reductions in tail length. Alternate plasmids were also cloned with a SphI restriction site between two-60A stretches, since published studies have demonstrated that similar constructs were more stable during subcloning and amplification in E. coli and produced mRNA with equivalent activity in mammalian cells. These alternate versions will be compared for activity using an in vitro editing assay across a range of CasX mRNA and gRNA doses to determine the consequential effects on editing activity. The sequences of the poly(A) tails described herein are listed in Table 24.

The sequences encoding the 5′ and 3′ UTRs, as well as the codons used for the CasX protein-coding sequences, are also critical for effective translation. UTRs were selected from annotated human gene transcripts based on genes (e.g., those encoding the α-globin, β-globin proteins) previously characterized to have high mRNA stability, as well as genes expected or previously demonstrated to be particularly well-expressed in the liver (i.e., genes encoding for the following proteins: albumin, complement 3, and cytochrome P450 2E1). The sequences of the 5′ and 3′ UTRs from these various genes are listed in Table 24. For the 3′ UTR, concatenations of individual 3′ UTRs were also tested. These constructs were cloned into plasmids containing a T7 promoter, CasX variant 515 or 676, and a poly(A) tail. To isolate the effects of individual 5′ and 3′ UTRs, each UTR was cloned into a construct that contained either the 3′ or 5′ α-globin UTR, respectively. IVTs will be performed and purified by binding to poly(dT) beads to capture full-length transcripts. The resulting mRNAs will initially be assessed by co-transfection with a B2M-targeting gRNA into HepG2 cells using a range of doses. Editing efficiency will be determined by HLA-immunostaining and flow cytometry as described in Example 6. The best-performing individual UTRs will be combined into various configurations, formulated into LNP, and tested in primary human hepatocytes and in mice.

Alternate codon optimizations are also being explored. In addition to the CasX codon optimization used for other delivery modalities, new versions were designed by building a codon usage table based on ribosomal protein codon usage and rebalancing CasX codon usage to match. In addition to potential improvements to the translation rate, this also effectively results in depletion of uracil bases, which may reduce immunogenicity. This codon optimization was also used for production of mRNAs. Additional codon usages have been designed using a variety of available codon optimization tools, adjusting settings as needed to achieve a range of GC content levels. These codon optimizations will be tested under a similar experimental design used for testing UTRs as described above, and the leading codon-optimized CasX candidates will be combined with leading UTR candidates to generate new CasX leads for further validation.

TABLE 24
List of encoding DNA sequences for the indicated elements used for the generation
and optimization of CasX mRNA

| | | |
|---|---|---|
| Description | Encoding Sequence | SEQ ID NO: |

Poly(A) tails

| | | |
|---|---|---|
| A₈₀ | AAAAAAAAAAAAAAAAAAAAAAAAAAAAAAAAAAAAAAAAAAAAAAAA | 14044 |
| | AAAAAAAAAAAAAAAAAAAAAAAAAAAAAAAA | |
| A₁₂₀ | AAAAAAAAAAAAAAAAAAAAAAAAAAAAAAAAAAAAAAAAAAAAAAAA | 14047 |
| | AAAAAAAAAAAAAAAAAAAAAAAAAAAAAAAAAAAAAAAAAAAAAAAA | |
| | AAAAAAAAAAAAAAAAAAAAAAAA | |
| A₆₀SphIA₆₀ | AAAAAAAAAAAAAAAAAAAAAAAAAAAAAAAAAAAAAAAAAAAAAAAA | 14048 |
| | AAAAAAAAAAAAGCATGCAAAAAAAAAAAAAAAAAAAAAAAAAAAAAA | |
| | AAAAAAAAAAAAAAAAAAAAAAAAAAAAAA | |

5′ UTRs

| | | |
|---|---|---|
| α-globin | ACTCTTCTGGTCCCCACAGACTCAGAGAGAACCC | 14049 |
| β-globin | ACATTTGCTTCTGACACAACTGTGTTCACTAGCAACCTCAAACA | 14050 |
| Albumin | CTAGCTTTTCTCTTCTGTCAACCCCACACGCCTTT | 14051 |
| Cytochrome | CTCCCGGGCTGGCAGCAGGGCCCCAGC | 14052 |
| P450 2E1 | | |
| (CYP2E1) | | |
| Complement 3 | ACTCCTCCCCATCCTCTCCCTCTGTCCCTCTGTCCCTCTGACCCTGCA | 14053 |
| (C3) | CTGTCCC | |

3′ UTRs

| | | |
|---|---|---|
| α-globin | GCTGGAGCCTCGGTGGCCATGCTTCTTGCCCCTTGGGCCTCCCCCCAG | 14046 |
| | CCCCTCCTCCCCTTCCTGCACCCGTACCCCCGTGGTCTTTGAATAAAG | |
| | TCTGAGTGGGCGGCA | |
| Albumin | CATCACATTTAAAAGCATCTCAGCCTACCATGAGAATAAGAGAAAGAA | 14054 |
| | AATGAAGATCAAAAGCTTATTCATCTGTTTTTCTTTTTCGTTGGTGTA | |
| | AAGCCAACACCCTGTCTAAAAAACATAAATTTCTTTAATCATTTTGCC | |
| | TCTTTTCTCTGTGCTTCAATTAATAAAAAATGGAAAGAATCTAATAGA | |
| | GTGGTACAGCACTGTTATTTTTCAAAGATGTGTTGCTATCCTGAAAAT | |
| | TCTGTAGGTTCTGTGGAAGTTCCAGTGTTCTCTCTTATTCCACTTCGG | |
| | TAGAGGATTTCTAGTTTCTTGTGGGCTAATTAAATAAATCATTAATAC | |
| | TCTTCTAAGTTATGGATTATAAACATTCAAAATAATATTTTGACATTA | |
| | TGATAATTCTGAATAAAAGAACAAAAACCA | |
| Albumin | GCATCACATTTAAAAGCATCTCAGCCTACCATGAGAATAAGAGAAAGA | 14055 |
| (truncated) | AAATGAAGATCAATAGCTTATTCATCTCTTTTTCTTTTTCGTTGGTGT | |
| | AAAGCCAACACCCTGTCTAAAAAACATAAATTTCTTTAATCATTTTGC | |
| | CTCTTTTCTCTGTGCTTCAATTAATAAAAAATGGAAAGAACCTAGATC | |
| | T | |
| β-globin | GCTCGCTTTCTTGCTGTCCAATTTCTATTAAAGGTTCCTTTGTTCCCT | 14056 |
| | AAGTCCAACTACTAAACTGGGGGATATTATGAAGGGCCTTGAGCATCT | |
| | GGATTCTGCCTAATAAAAAACATTTATTTTCATTGCAA | |
| α-globin + β- | GCTGGAGCCTCGGTGGCCATGCTTCTTGCCCCTTGGGCCTCCCCCCAG | 14057 |
| globin | CCCCTCCTCCCCTTCCTGCACCCGTACCCCCGTGGTCTTTGAATAAAG | |
| | TCTGAGTGGGCGGCAGCTCGCTTTCTTGCTGTCCAATTTCTATTAAAG | |
| | GTTCCTTTGTTCCCTAAGTCCAACTACTAAACTGGGGGATATTATGAA | |
| | GGGCCTTGAGCATCTGGATTCTGCCTAATAAAAAACATTTATTTTCAT | |
| | TGCAA | |
| β-globin + α- | GCTCGCTTTCTTGCTGTCCAATTTCTATTAAAGGTTCCTTTGTTCCCT | 14058 |
| globin | AAGTCCAACTACTAAACTGGGGGATATTATGAAGGGCCTTGAGCATCT | |
| | GGATTCTGCCTAATAAAAAACATTTATTTTCATTGCAAGCTGGAGCCT | |
| | CGGTGGCCATGCTTCTTGCCCCTTGGGCCTCCCCCCAGCCCCTCCTCC | |
| | CCTTCCTGCACCCGTACCCCCGTGGTCTTTGAATAAAGTCTGAGTGGG | |
| | CGGCA | |

### Example 8: Demonstration of In Vivo Editing and Assessment of AAV Biodistribution in the Liver Tissue

Experiments were performed to demonstrate that systems of small CRISPR proteins such as CasX and gRNA are capable of editing liver cells when expressed from an AAV episome in vivo. The biodistribution of XAAVs (AAVs encoding CasX and gRNA) within the liver tissue was also assessed.

### Materials and Methods

### AAV Construct Cloning:

AAV constructs containing a UbC promoter driving CasX variant 491 expression and a Pol III U6 promoter driving gRNA scaffold 235 with a ROSA26-targeting spacer were generated using standard molecular cloning techniques. The sequences of the AAV constructs are listed in Table 25. Cloned and sequence-validated AAV constructs were maxi-prepped and subjected to quality assessment prior to transfection into HEK293T cells.

TABLE 25
Sequences of the AAV construct used for in vivo administration.*

| | Component | | |
|---|---|---|---|
| Construct ID | Name | DNA Sequence | SEQ ID NO: |
| 5 | 5′ ITR | CCTGCAGGCAGCTGCGCGCTCGCTCGCTCACTGAG | 13778 |
| | | GCCGCCCGGGCGTCGGGCGACCTTTGGTCGCCCGG | |
| | | CCTCAGTGAGCGAGCGAGCGCGCAGAGAGGGAGTG | |
| | | GCCAACTCCATCACTAGGGGTTCCT | |
| | buffer sequence | GCGGCCTCTAGACTCGAGGCGTT | 13974 |
| | UbC promoter | GGCCTCCGCGCCGGGTTTTGGCGCCTCCCGCGGGC | 13975 |
| | | GCCCCCCTCCTCACGGCGAGCGCTGCCACGTCAGA | |
| | | CGAAGGGCGCAGCGAGCGTCCTGATCCTTCCGCCC | |
| | | GGACGCTCAGGACAGCGGCCCGCTGCTCATAAGAC | |
| | | TCGGCCTTAGAACCCCAGTATCAGCAGAAGGACAT | |
| | | TTTAGGACGGGACTTGGGTGACTCTAGGGCACTGG | |
| | | TTTTCTTTCCAGAGAGCGGAACAGGCGAGGAAAAG | |
| | | TAGTCCCTTCTCGGCGATTCTGCGGAGGGATCTCC | |
| | | GTGGGGCGGTGAACGCCGATGATTATATAAGGACG | |
| | | CGCCGGGTGTGGCACAGCTAGTTCCGTCGCAGCCG | |
| | | GGATTTGGGTCGCGGTTCTTGTTTGTGGATCGCTG | |
| | | TGATCGTCACTTGGT | |
| | buffer sequence | CTCTGGCTAACTACCGGT | 13976 |
| | Kozak | GCCACC | 13977 |
| | start codon | ATGGCC | 13978 |
| | cMyc NLS | CCAGCGGCCAAACGGGTGAAGCTGGAC | 13979 |
| | linker | TCTAGA | 13980 |
| | CasX 491 | CAAGAGATCAAGAGAATCAACAAGATCAGAAGGAG | 13981 |
| | | ACTGGTCAAGGACAGCAACACAAAGAAGGCCGGCA | |
| | | AGACAGGCCCCATGAAAACCCTGCTCGTCAGAGTG | |
| | | ATGACCCCTGACCTGAGAGAGCGGCTGGAAAACCT | |
| | | GAGAAAGAAGCCCGAGAACATCCCTCAGCCTATCA | |
| | | GCAACACCAGCAGGGCCAACCTGAACAAGCTGCTG | |
| | | ACCGACTACACCGAGATGAAGAAAGCCATCCTGCA | |
| | | CGTGTACTGGGAAGAGTTCCAGAAAGACCCCGTGG | |
| | | GCCTGATGAGCAGAGTTGCTCAGCCTGCCAGCAAG | |
| | | AAGATCGACCAGAACAAGCTGAAGCCCGAGATGGA | |
| | | CGAGAAGGGCAATCTGACCACAGCCGGCTTTGCCT | |
| | | GCTCTCAGTGTGGCCAGCCTCTGTTCGTGTACAAG | |
| | | CTGGAACAGGTGTCCGAGAAAGGCAAGGCCTACAC | |
| | | CAACTACTTCGGCAGATGTAACGTGGCCGAGCACG | |
| | | AGAAGCTGATTCTGCTGGCCCAGCTGAAACCTGAG | |
| | | AAGGACTCTGATGAGGCCGTGACCTACAGCCTGGG | |
| | | CAAGTTTGGACAGAGAGCCCTGGACTTCTACAGCA | |
| | | TCCACGTGACCAAAGAAAGCACACACCCCGTGAAG | |
| | | CCCCTGGCTCAGATCGCCGGCAATAGATACGCCTC | |
| | | TGGACCTGTGGGCAAAGCCCTGTCCGATGCCTGCA | |
| | | TGGGAACAATCGCCAGCTTCCTGAGCAAGTACCAG | |
| | | GACATCATCATCGAGCACCAGAAGGTGGTCAAGGG | |
| | | CAACCAGAAGAGACTGGAAAGCCTGAGGGAGCTGG | |
| | | CCGGCAAAGAGAACCTGGAATACCCCAGCGTGACC | |
| | | CTGCCTCCTCAGCCTCACACAAAAGAAGGCGTGGA | |
| | | CGCCTACAACGAAGTGATCGCCAGAGTGAGAATGT | |
| | | GGGTCAACCTGAACCTGTGGCAGAAGCTGAAACTG | |
| | | TCCAGGGACGACGCCAAGCCTCTGCTGAGACTGAA | |
| | | GGGCTTCCCTAGCTTCCCTCTGGTGGAAAGACAGG | |
| | | CCAATGAAGTGGATTGGTGGGACATGGTCTGCAAC | |
| | | GTGAAGAAGCTGATCAACGAGAAGAAAGAGGATGG | |
| | | CAAGGTTTTCTGGCAGAACCTGGCCGGCTACAAGA | |
| | | GACAAGAAGCCCTGAGGCCTTACCTGAGCAGCGAA | |
| | | GAGGACCGGAAGAAGGGCAAGAAGTTCGCCAGATA | |
| | | CCAGCTGGGCGACCTGCTGCTGCACCTGGAAAAGA | |
| | | AGCACGGCGAGGACTGGGGCAAAGTGTACGATGAG | |
| | | GCCTGGGAGAGAATCGACAAGAAGGTGGAAGGCCT | |
| | | GAGCAAGCACATTAAGCTGGAAGAGGAAAGAAGGA | |
| | | GCGAGGACGCCCAATCTAAAGCCGCTCTGACCGAT | |
| | | TGGCTGAGAGCCAAGGCCAGCTTTGTGATCGAGGG | |
| | | CCTGAAAGAGGCCGACAAGGACGAGTTCTGCAGAT | |
| | | GCGAGCTGAAGCTGCAGAAGTGGTACGGCGATCTG | |
| | | AGAGGCAAGCCCTTCGCCATTGAGGCCGAGAACAG | |
| | | CATCCTGGACATCAGCGGCTTCAGCAAGCAGTACA | |
| | | ACTGCGCCTTCATTTGGCAGAAAGACGGCGTCAAG | |
| | | AAACTGAACCTGTACCTGATCATCAATTACTTCAA | |
| | | AGGCGGCAAGCTGCGGTTCAAGAAGATCAAACCCG | |
| | | AGGCCTTCGAGGCTAACAGATTCTACACCGTGATC | |
| | | AACAAAAAGTCCGGCGAGATCGTGCCCATGGAAGT | |
| | | GAACTTCAACTTCGACGACCCCAACCTGATTATCC | |
| | | TGCCTCTGGCCTTCGGCAAGAGACAGGGCAGAGAG | |
| | | TTCATCTGGAACGATCTGCTGAGCCTGGAAACCGG | |
| | | CTCTCTGAAGCTGGCCAATGGCAGAGTGATCGAGA | |
| | | AAACCCTGTACAACAGGAGAACCAGACAGGACGAG | |
| | | CCTGCTCTGTTTGTGGCCCTGACCTTCGAGAGAAG | |
| | | AGAGGTGCTGGACAGCAGCAACATCAAGCCCATGA | |
| | | ACCTGATCGGCGTGGACCGGGGCGAGAATATCCCT | |
| | | GCTGTGATCGCCCTGACAGACCCTGAAGGATGCCC | |
| | | ACTGAGCAGATTCAAGGACTCCCTGGGCAACCCTA | |
| | | CACACATCCTGAGAATCGGCGAGAGCTACAAAGAG | |
| | | AAGCAGAGGACAATCCAGGCCAAGAAAGAGGTGGA | |
| | | ACAGCGCAGAGCCGGCGGATACTCTAGGAAGTACG | |
| | | CCAGCAAGGCCAAGAATCTGGCCGACGACATGGTC | |
| | | CGAAACACCGCCAGAGATCTGCTGTACTACGCCGT | |
| | | GACACAGGACGCCATGCTGATCTTCGAGAATCTGA | |
| | | GCAGAGGCTTCGGCCGGCAGGGCAAGAGAACCTTT | |
| | | ATGGCCGAGAGGCAGTACACCAGAATGGAAGATTG | |
| | | GCTCACAGCTAAACTGGCCTACGAGGGACTGAGCA | |
| | | AGACCTACCTGTCCAAAACACTGGCCCAGTATACC | |
| | | TCCAAGACCTGCAGCAATTGCGGCTTCACCATCAC | |
| | | CAGCGCCGACTACGACAGAGTGCTGGAAAAGCTCA | |
| | | AGAAAACCGCCACCGGCTGGATGACCACCATCAAC | |
| | | GGCAAAGAGCTGAAGGTTGAGGGCCAGATCACCTA | |
| | | CTACAACAGGTACAAGAGGCAGAACGTCGTGAAGG | |
| | | ATCTGAGCGTGGAACTGGACAGACTGAGCGAAGAG | |
| | | AGCGTGAACAACGACATCAGCAGCTGGACAAAGGG | |
| | | CAGATCAGGCGAGGCTCTGAGCCTGCTGAAGAAGA | |
| | | GGTTTAGCCACAGACCTGTGCAAGAGAAGTTCGTG | |
| | | TGCCTGAACTGCGGCTTCGAGACACACGCCGATGA | |
| | | ACAGGCTGCCCTGAACATTGCCAGAAGCTGGCTGT | |
| | | TCCTGAGAAGCCAAGAGTACAAGAAGTACCAGACC | |
| | | AACAAGACCACCGGCAACACCGACAAGAGGGCCTT | |
| | | TGTGGAAACCTGGCAGAGCTTCTACAGAAAAAAGC | |
| | | TGAAAGAAGTCTGGAAGCCCGCCGTG | |
| | linker | GGCTCC | 14059 |
| | cMyc NLS | CCCGCCGCGAAGCGAGTGAAACTGGAC | 14060 |
| | stop codon | TAA | — |
| | buffer sequence | GAATTCCTAGAGCTCGCTGATCAGCCTCGA | 14061 |
| | bGH poly(A) | CTGTGCCTTCTAGTTGCCAGCCATCTGTTGTTTGC | 13988 |
| | | CCCTCCCCCGTGCCTTCCTTGACCCTGGAAGGTGC | |
| | | CACTCCCACTGTCCTTTCCTAATAAAATGAGGAAA | |
| | | TTGCATCGCATTGTCTGAGTAGGTGTCATTCTATT | |
| | | CTGGGGGGTGGGGTGGGGCAGGACAGCAAGGGGGA | |
| | | GGATTGGGAAGAGAATAGCAGGCATGCTGGGGA | |
| | buffer sequence | GGTACCGT | 14062 |
| | U6 promoter | GAGGGCCTATTTCCCATGATTCCTTCATATTTGCA | 13990 |
| | | TATACGATACAAGGCTGTTAGAGAGATAATTGGAA | |
| | | TTAATTTGACTGTAAACACAAAGATATTAGTACAA | |
| | | AATACGTGACGTAGAAAGTAATAATTTCTTGGGTA | |
| | | GTTTGCAGTTTTAAAATTATGTTTTAAAATGGACT | |
| | | ATCATATGCTTACCGTAACTTGAAAGTATTTCGAT | |
| | | TTCTTGGCTTTATATATCTTGTGGAAAGGAC | |
| | buffer sequence | GAAACACC | 13991 |
| | Scaffold 235 | ACTGGCGCTTCTATCTGATTACTCTGAGCGCCATC | 13992 |
| | | ACCAGCGACTATGTCGTAGTGGGTAAAGCCGCTTA | |
| | | CGGACTTCGGTCCGTAAGAGGCATCAGAG | |
| | Spacer 35.2 | AGAAGATGGGCGGGAGTCTT | 14063 |
| | buffer sequence | TTTTTGGCGGCCGC | 14064 |
| | 3′ ITR | AGGAACCCCTAGTGATGGAGTTGGCCACTCCCTCT | 13779 |
| | | CTGCGCGCTCGCTCGCTCACTGAGGCCGGGCGACC | |
| | | AAAGGTCGCCCGACGCCCGGGCTTTGCCCGGGCGG | |
| | | CCTCAGTGAGCGAGCGAGCGCGCAGCTGCCTGCAG | |
| | | G | |

*Components are listed in a 5′ to 3′ order within the constructs

AAV production and titer determination were performed as described in Example 5.

To quantify AAV-mediated genome editing in vivo, 3E9, 3E10, or 3E11 AAV particles containing CasX protein 491 and guide scaffold variant 235 with spacer 35.2 targeting the safe harbor ROSA26 locus (construct #5; refer to Table 25 for sequences) were administered intravenously via retro-orbital sinus to adult two-month-old C57BL/6J mice. Mice were observed for five minutes after injection to ensure recovery from anesthesia before being placed into their home cage. Naïve, untreated mice served as experimental controls. Either 1 week or four weeks post-injection, mice were euthanized. Liver and other tissues were harvested for gDNA extraction using the Zymo Quick DNA/RNA miniprep Kit following the manufacturer's instructions. Target amplicons were then amplified from 200 ng of extracted gDNA with a set of primers targeting the mouse ROSA26 locus and processed as described earlier in Example 4 for editing assessment by NGS.

### Results

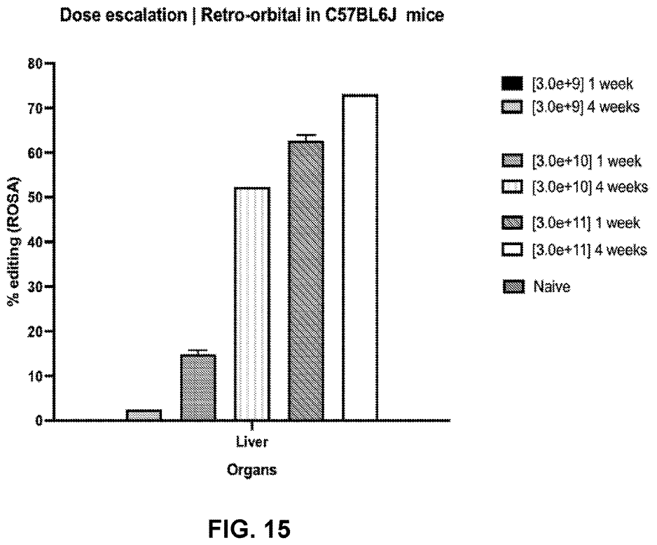

The precent editing in the ROSA26 locus is shown in FIG. 15. Analysis of editing outcomes confirms that AAV is an effective vehicle for delivering therapeutic payloads to the liver. While low doses of AAV (3E9) only edited the liver at ~2%, ~52% editing rate was found in medium dose (3E10) at 4 weeks and ~65% and 75% editing was found in the high dose 3E11 groups at 1 and 4 weeks, respectively.

Example 9: Demonstration that CasX mRNA and Targeting gRNA can be Delivered Via LNPs to Achieve In Vivo Targeting

Experiments were performed to demonstrate that CasX molecules and guides can edit the ROSA26 locus in the liver in vivo when delivered via LNPs.

### Materials and Methods

### Mouse Lines:

Mouse strains including C57BL/6 inbred strains from Jackson Labs (C57BL/6J) and Charles River Labs (C57BL/6N) were used to assess CasX-mediated editing in vivo. Males and females between 4 and 8 weeks of age at the time of injection were used.

### LNP Formulation:

LNP co-formulations were performed as described in Example 12. CasX 676 mRNA constructs (see Table 20 for sequences) and a gRNA using scaffold 174 with a spacer targeting ROSA26 (AGAAGAUGGGGGGAGUCUU; SEQ ID NO: 14123) were encapsulated within the same LNP using a 1:1 mass ratio for mRNA:gRNA. The v1 chemical modification profile (as discussed in Example 6) was applied to the gRNA scaffold 174 with the targeting spacer. Formulated LNPs were buffer-exchanged to PBS for in vivo injection. At the time of injection, concentrated LNPs were mixed with sterile-filtered PBS.

### In Vivo Administration of Formulated LNPs:

Mice were anesthetized via isoflurane inhalation and anesthesia was verified by the loss of righting reflex and toe pinch. LNPs were administered intravenously, either through the retro-orbital sinus or lateral tail vein. Mice were observed for five minutes after injection to ensure recovery from anesthesia before being placed into their home cage. Mice were randomized into groups at the following doses: 0.3 mg/kg, 1.0 mg/kg, or 3.0 mg/kg. Naïve, uninjected animals served as experimental controls.

Mice were monitored daily for 7 days; after 7 days, mice were euthanized, and various tissues (liver, spleen, heart, and kidney) were harvested from each animal. Harvested tissues were processed for genomic DNA (gDNA) and RNA using the Zymo Quick DNA/RNA Miniprep kit following the manufacturer's instructions. Target amplicons were then amplified from the extracted gDNA with a set of primers targeting the mouse ROSA26 locus and processed as described earlier in Example 4 for editing assessment by NGS.

### Results

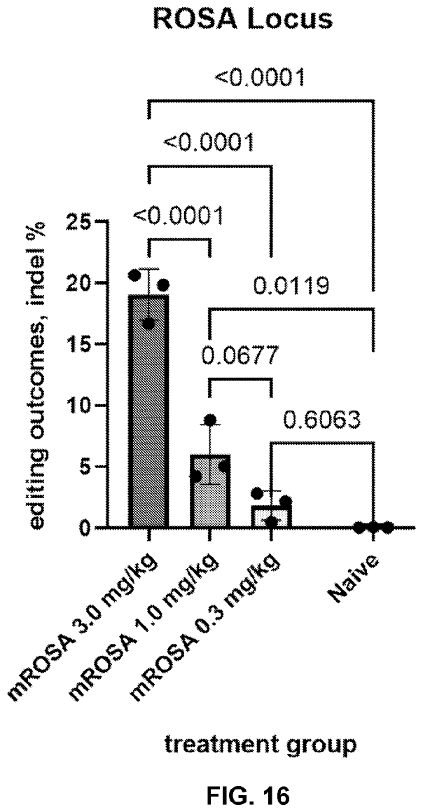

The bar plot in FIG. 16 shows the editing results, as percent editing measured as indel rate, at the ROSA26 locus in mice treated with LNPs co-formulated with CasX 676 mRNA #1 and a ROSA26-targeting gRNA. As illustrated in FIG. 16, a dose-dependent effect was observed with editing at the ROSA26 locus, with the 3.0 and 1.0 mg/kg doses resulting in a statistically significant difference in editing compared to the editing level achieved with the naïve control group.

The results of the experiment show that delivery of CasX mRNA and targeting gRNA via LNPs can induce editing in the liver. These findings demonstrate the efficacy of the CasX:gRNA system using mRNA delivery in LNPs to the liver, a target tissue of therapeutic relevance.

Example 10: Assessment of CasX Variants 491, 515, 528, 593, 676, and 690 and gRNAs Using Scaffold Variant 235 with PCSK9-Targeting Spacers to Induce Off-Target Editing

Experiments were performed to assess the potential off-target editing of various CasX proteins and gRNAs with spacers designed to target the human PCSK9 locus. Here, in silico analysis was performed to determine and assess the predicted off-target sites for the various PCSK9-targeting spacers. Furthermore, cut site incorporation and sequencing (CSI-seq) was performed in cell assays assessing several CasX variants individually complexed with a gRNA using scaffold variant 235 with individual human PCSK9-targeting spacers. The CSI-seq assay involves introducing a plasmid encoding a CasX variant and gRNA scaffold with a PCSK9-targeting spacer, along with a double-stranded oligodeoxynucleotide (dsODN) donor to identify sites of double-strand breaks (DSBs) that are believed to occur as a result of CasX:gRNA activity. These sites are identified using unidirectional sequencing of the dsODN sequence. Further CSI-seq assays are performed to identify the off-target profiles of PCSK9-targeting spacers and CasX proteins, as described in later examples.

### Materials and Methods

In Silico Prediction of Off-Target Sites for gRNA Spacers:

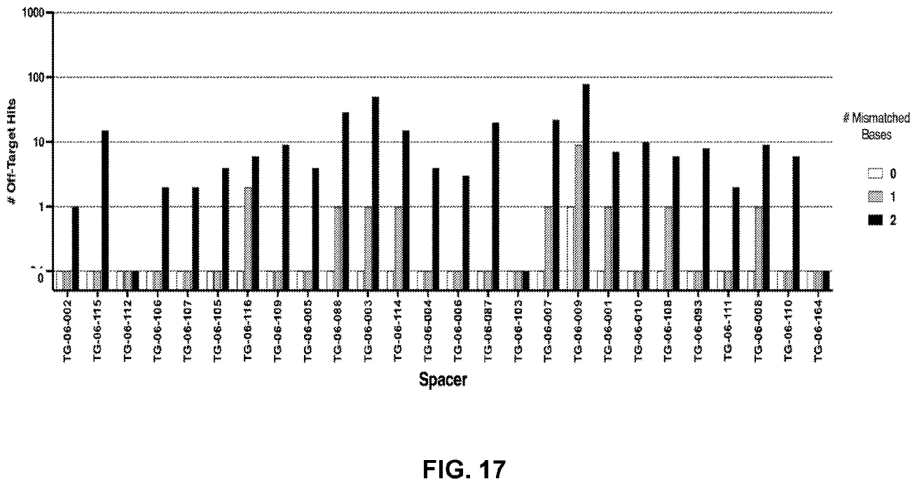

Computational off-target prediction analysis was performed on 26 PCSK9-targeting spacers selected for screening and assessment of editing activity at the human PCSK9 locus. Cas-OFFinder (v. 3.0.0b3) was used to generate lists of predicted off-target sites on human genome reference build hg38 (GRCh38), using an ‘NTC’ PAM input string, with a maximum of two mismatches and no RNA/DNA bulges per predicted off-target site. Tabulated hits were counted for each spacer. The results are displayed in FIG. 17.

### CSI-Seq Assay:

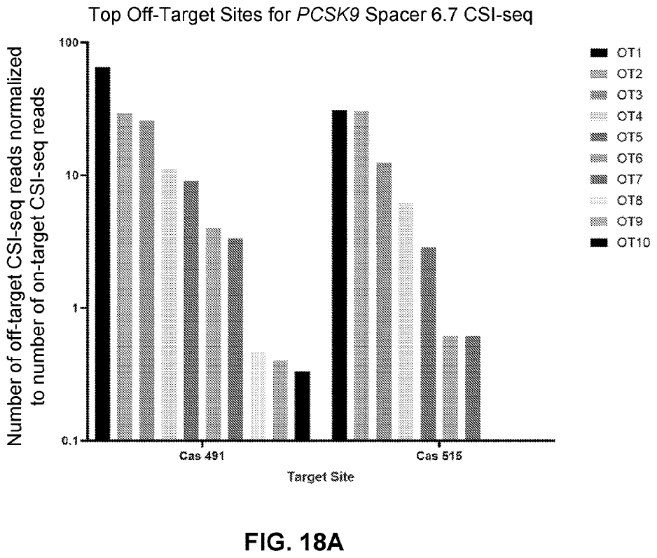

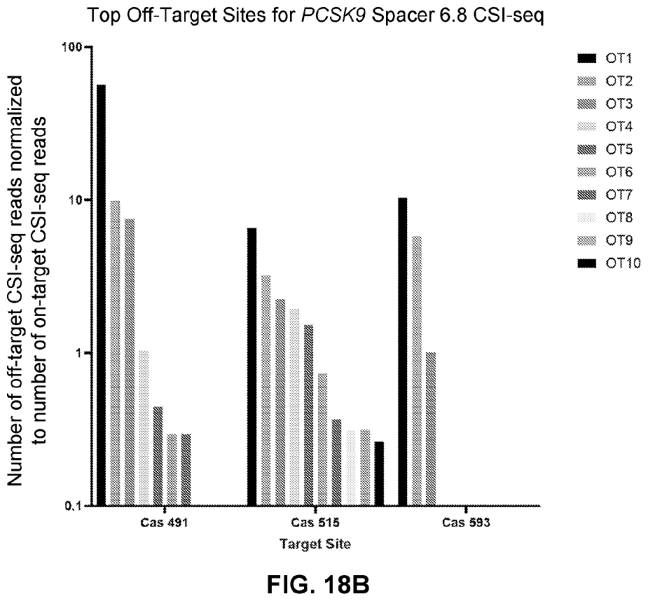

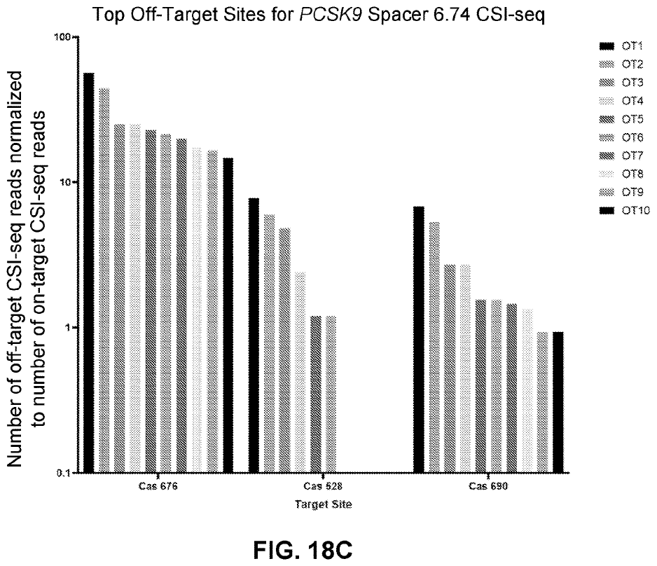

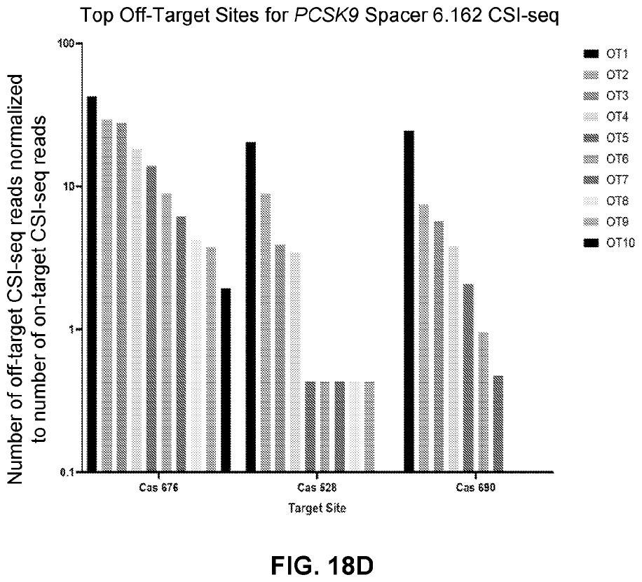

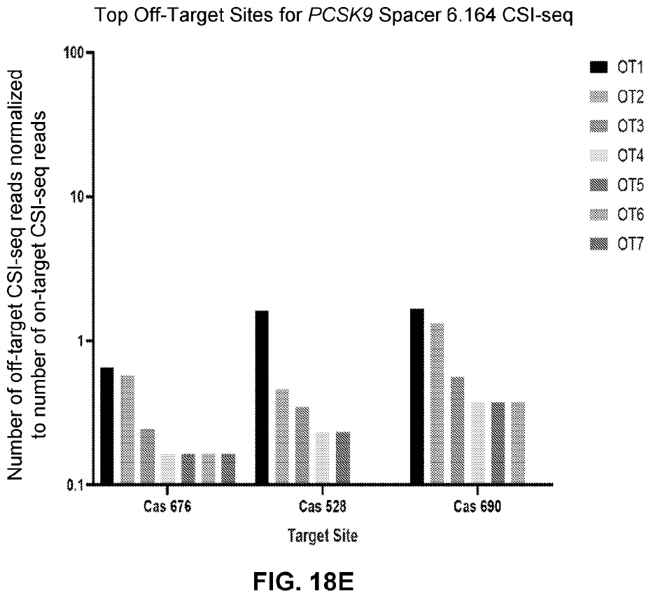

~700,000 HEK293 cells were nucleofected using a Lonza nucleofector with 500 ng of plasmid DNA encoding the CasX variant, gRNA with PCSK9-targeting spacers (sequences listed in Table 26), and 100 nmol of dsODN. Specifically, for PCSK9-targeting spacer 6.7, CasX variants 491 and 515 were assessed; for spacer 6.8, CasX 491, 515, and 593 were assessed; and for spacers 6.74, 6.162, and 6.164, CasX 528, 676, and 690 were assessed. Nucleofected cells were then plated onto a 6-well plate and grown for five days, after which they were harvested for genomic DNA (gDNA) extraction using a Zymo QuickDNA™ miniprep kit. Briefly, for CSI-seq library preparation, gDNA was randomly fragmented and ligated with adaptors using the Tn5 transposase, followed by PCR amplification via nested PCR1 and PCR2 reactions. For PCR1, an adaptor-specific primer and a primer binding to the dsODN in the forward or reverse direction with a read 1 primer binding site were used; for PCR2, the same adaptor-specific primer and a primer to the read 1 primer binding site were used. Samples were normalized and sequenced on an Illumina NextSeq™. Subsequently, samples were analyzed to identify off-target editing events. Briefly, reads were filtered for presence of the full dsODN sequence to eliminate off-target priming sites from analysis. These filtered, dsODN-containing reads are referred to herein as “CSI-seq reads.” Genomic sites with multiple mapped reads were required to have aligned reads in both orientations to further reduce false positives. Sites with a nearby sequence containing seven or fewer mismatches or RNA/DNA bulges away from the search spacer were identified as potential off-target sites. The number of CSI-seq reads at each site relative to the number of CSI-seq reads at the on-target site was expected to be roughly proportional to the relative amount of off-target editing at that site. FIGS. 18A-18E present the level of off-target editing of the tested spacers at the given off-target sites normalized to the level of on-target editing for each spacer, calculated as the number of off-target CSI-seq reads for a spacer at a particular site divided by the number of on-target CSI-reads for the spacer, multiplied by 100.

TABLE 26
Sequences of human PCSK9-targeting spacers tested in a CSI-seq assay

| | | | | SEQ ID |
|---|---|---|---|---|
| Spacer ID | Spacer DNA sequence | SEQ ID NO: | Spacer RNA sequence | NO: |
| 6.7 | TCCTGGCTTCCTGGTGAAGA | 13788 | UCCUGGCUUCCUGGUGAAGA | 552 |
| 6.8 | TGGCTTCCTGGTGAAGATGA | 13789 | UGGCUUCCUGGUGAAGAUGA | 569 |
| 6.74 | CCGGCGGGCAGCCTGGGCCT | 13806 | CCGGCGGGCAGCCUGGGCCU | 5538 |
| 6.162 | CCTAGACACCAGCATACAGA | 13848 | CCUAGACACCAGCAUACAGA | 5711 |
| 6.164 | GGAAGTGCCATTCCCAAAAA | 13850 | GGAAGUGCCAUUCCCAAAAA | 5542 |

### Results

In silico analysis using Cas-OFFinder was performed to determine the predicted off-target sites for the 26 PCSK9-targeting spacers that were being assessed for editing activity, and the data are illustrated in FIG. 17. The data show that the screened spacers exhibited favorable predicted off-target profiles, with all spacers having no perfectly matched off-target sites. Several spacers were predicted to have several off-target sites with 1 bp mismatch, and unsurprisingly, the number of predicted off-target sites increased when the parameter was adjusted to encompass 2 bp mismatch hits.

Furthermore, potential off-target sites were identified using a CSI-seq assay for each PCSK9-targeting spacer and the indicated CasX variants. The results are depicted as the number of off-target CSI-seq reads at a particular off-target sitenormalized to the number of on-target CSI-seq reads obtained that spacer, as described above (FIGS. 18A-18E). Spacers with few off-target sites and relatively few CSI-seq reads at each site were considered to have minimal off-target risk, while spacers with many off-target sites with moderate numbers of CSI-seq reads or a top off-target site with a high number of reads were considered to have a high off-target risk. As illustrated in FIGS. 18A-18E, the range of off-target sites identified for each spacer varied widely, both in terms of the number of sites identified as well as their relative frequency. As expected, different CasX variants had distinct off-target profiles as well due to their relative tolerance for mismatches within the spacer and their different PAM preferences, which affect the available off-target landscape.

Several spacers had a top off-target site with reads >30% of on-target, with one with a top off-target with 65% of on-target CSI-seq reads. For many of these high off target spacers, when paired with more CasX variants with higher PAM stringency or increased global specificity, these off target rates dropped nearly 10-fold (spacers 6.7, 6.8, and 6.74). In addition to reducing overall off target effects, the off-target sites of a given spacer were not always conserved across editing molecule variants and many that were conserved dramatically changed their prevalence relative to other off target sites.

Spacer 6.174 performed better, with a top off-target rate of 1.7%. Less dramatic changes in off target rate across CasX variants were observed for this spacer, though one pairing reduced the top off target rate to 0.7%. The top off-target site was very often not the identified site with the fewest mismatches, and in many cases sites with fewer mismatches could be identified computationally but did not appear in the CSI-seq hits. This observation, consistent with published data for other CRISPR nucleases, demonstrates the importance of experimentally determining the potential off-target editing locations for spacers. These results show that at least one spacer with minimal off-target editing effects has been identified for PCSK9, enabling the safe and efficacious targeting of the locus. Additionally, these results indicate that CasX molecules engineered for higher PAM stringency and global specificity can improve the off-target profiles of spacers that would otherwise be unsuitable for therapeutic use.

### Example 11: CpG-Depletion of DNA Encoding the Guide RNA Scaffold Improves CasX-Mediated Editing In Vitro

Pathogen-associated molecular patterns (PAMPs), such as unmethylated CpG motifs, are small molecular motifs conserved within a class of microbes. They are recognized by toll-like receptors (TLRs) and other pattern recognition receptors in eukaryotes and often induce a non-specific immune activation. In the context of gene therapy, therapeutics containing PAMPs are often not as well-tolerated and are rapidly cleared from the patient given the strong immune response triggered, which ultimately leads to reduced therapeutic efficacy. CpG motifs are short single-stranded DNA sequences containing the dinucleotide CG. When these CpG motifs are unmethylated, they act as PAMPs and therefore stimulate the immune response. In this example, experiments were performed to deplete CpG motifs in the guide scaffold coding sequence in the context of an AAV construct encoding CasX variant 491, guide scaffold variant 235, and spacer 7.37 targeting the endogenous B2M (beta-2-microglobulin) locus, and test the effect of CpG-depletion in the guide scaffold on editing of the B2M locus in vitro.

### Materials and Methods

### Design of CpG-Depleted Guide Scaffolds:

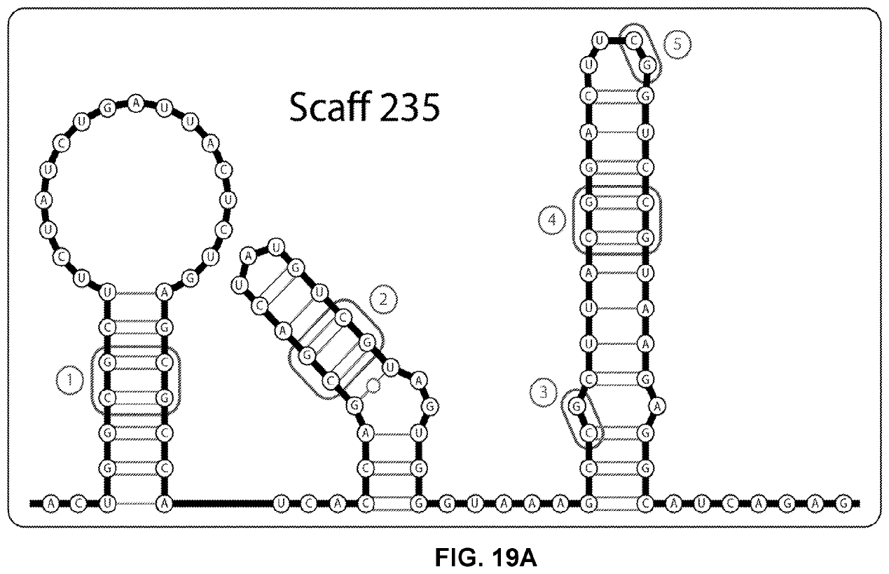

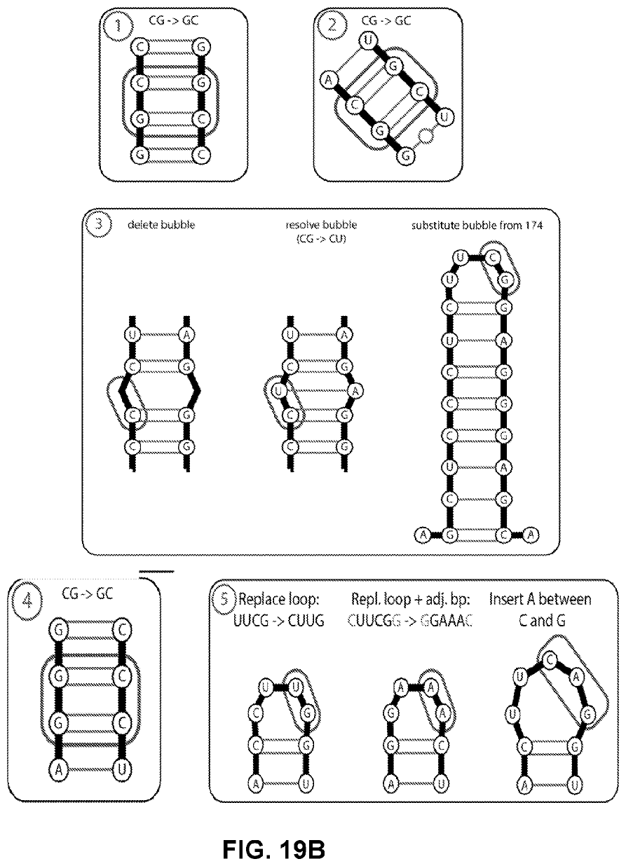

Nucleotide substitutions were rationally-designed to replace native CpG motifs within the base gRNA scaffold variant (gRNA scaffold 235) with the intent to preserve editing activity while reducing scaffold immunogenicity. It was believed that as many CpG-motifs as possible should be removed from the scaffold coding sequence in order to sufficiently reduce immunogenicity. Scaffold 235 contains a total of eight CpG elements; six of which are predicted to basepair and form complementary strands of a double-stranded secondary structure (see FIG. 19A). Therefore, the six basepairing CpGs forming three pairs were mutated in concert to maintain important secondary structures. This reduced the number of independent CpG-containing regions to five (three pairs and two single CpGs) to be considered independently for CpG-removal. Specifically, mutations were designed in (1) the pseudoknot stem, (2) the scaffold stem, (3) the extended stem bubble, (4) the extended step, and (5) the extended stem loop, as diagrammed in FIG. 19B and described in detail below.

In the pseudoknot stem (region 1), the CpG pair was flipped to a GpC to minimize the alteration of the base composition and sequence. Based on previous experiments involving replacing individual base pairs, it was anticipated that this mutation was not likely to be detrimental to the structure and function of the guide RNA scaffold.

Similarly, in the scaffold stem (region 2) the CpG pair was flipped to a GpC to minimize the alteration of the base composition and sequence. It was anticipated that this mutation was likely to be detrimental to the structure and function of the guide RNA scaffold because strong sequence conservation was seen in this region in previous experiments mutating individual bases or base pairs. This strong sequence conservation is likely due to the scaffold stem loop being important in interacting with the CasX protein as well as in the formation of a triplex structural element with the pseudoknot region.

In the extended stem bubble (region 3) the single CpG was removed by one of three strategies. First, the bubble was deleted by mutating CG->C. Second, the bubble was resolved to restore ideal basepairing by mutating CG->CT. Third, the entire extended stem loop was replaced with the extended stem loop of scaffold 174. Note that, by itself, the replacement of the extended stem loop with that of scaffold 174 recapitulates scaffold 316, which has previously been shown to edit efficiently. There are no CpG motifs in the extended stem loop of scaffold 174. Therefore, replacing the extended stem loop with that of scaffold 174 also removes the CpG motif in the extended stem (region 4). Based on previous experiments showing the relative robustness of the extended stem to small changes, it was anticipated that mutating the extended stem bubble was moderately likely to be detrimental to the structure and function of the guide RNA scaffold.

In the extended stem (region 4), the CpG pair could not be flipped to GpC without generating additional CpG motifs. Therefore, the CpGs were changed to a GG and a complementary CC motif. Similar to region 3, based on the relative robustness of the extended stem to small changes, it was anticipated that this mutation was not likely to be detrimental to the structure and function of the guide RNA scaffold.

Finally, the extended stem loop (region 5) was mutated in one of three ways that were designed based on previous experiments examining the stability of the stem loop. In particular, several variations of the stem loop had previously been shown to have similar stability levels, and some of these variations of the stem loop do not contain CpGs. Based on these findings, first, the loop was replaced with a new loop with a CUUG sequence. Second, the loop was replaced with a new loop with a GAAA sequence. Since the GAAA loop replacement would generate a novel CpG adjacent to the loop, it was combined with a C->G base swap and the corresponding G->C base swap on the complementary strand, ultimately resulting in a CUUCGG->GGAAAC exchange. Third, the loop was mutated by the insertion of an A to interrupt the CpG motif and thereby increase the size of the loop from 4 to 5 bases. It was anticipated that randomly mutating the extended stem loop would likely have detrimental effects on secondary structure stability and hence on editing. However, relying on previously confirmed sequences was believed to have a lower risk associated with a replacement.

To generate guide RNA scaffolds encoded by DNA with reduced CpG levels, the mutations described above were combined in various configurations. Table 27, below, summarizes combinations of the mutations that were used. In Table 27, a 0 indicates that no mutation was introduced to a given region, a 1, 2, or 3 indicates that a mutation was introduced in that region, as diagrammed in FIG. 19B, and n/a indicates not applicable. Specifically, for region 1, the pseudoknot stem, a 1 indicates that a CG->GC mutation was introduced. For region 2, the scaffold stem, a 1 indicates that a CG->GC mutation was introduced. For region 3, the extended stem bubble, a 1 indicates that the bubble was removed by the deletion of the G and A bases that form the bubble, a 2 indicates that the bubble was resolved by a CG->CU mutation that allows for basepairing between the A and U bases, and a 3 indicates that the extended stem loop was replaced with the extended step loop from guide scaffold 174. For region 4, the extended stem, a 1 indicates that a CG->GC mutation was introduced. For region 5, the extended stem loop, a 1 indicates that the loop was replaced from UUCG->CUUG, a 2 indicates that the loop was replaced along with a basepair adjacent to the loop, from CUUCGG->GGAAAC, and a 3 indicates that an A was inserted between the C and the G.

TABLE 27
Summary of mutations for CpG-reduction
and depletion in guide scaffold 235

| | Region 1 | Region 2 | Region 3 | Region 4 | Region 5 |
|---|---|---|---|---|---|
| Scaffold | (Pseudoknot | (Scaffold | (Extended | (Extended | (Extended |
| ID | stem) | stem) | stem bubble) | stem) | stem loop) |
| 320 | 1 | 0 | 0 | 1 | 0 |
| 321 | 1 | 0 | 1 | 1 | 0 |
| 322 | 1 | 0 | 2 | 1 | 0 |
| 323 | 1 | 0 | 3 | n/a | 0 |
| 324 | 1 | 0 | 1 | 1 | 1 |
| 325 | 1 | 0 | 2 | 1 | 1 |
| 326 | 1 | 0 | 3 | n/a | 1 |
| 327 | 1 | 0 | 1 | 1 | 2 |
| 328 | 1 | 0 | 2 | 1 | 2 |
| 329 | 1 | 0 | 3 | n/a | 2 |
| 330 | 1 | 0 | 1 | 1 | 3 |
| 331 | 1 | 0 | 2 | 1 | 3 |
| 332 | 1 | 0 | 3 | n/a | 3 |
| 334 | 1 | 1 | 2 | 1 | 1 |
| 335 | 1 | 1 | 3 | n/a | 1 |
| 336 | 1 | 1 | 1 | 1 | 2 |
| 337 | 1 | 1 | 2 | 1 | 2 |
| 338 | 1 | 1 | 3 | n/a | 2 |
| 339 | 1 | 1 | 1 | 1 | 3 |
| 340 | 1 | 1 | 2 | 1 | 3 |
| 341 | 1 | 1 | 3 | n/a | 3 |
| 235 | 0 | 0 | 0 | 0 | 0 |

Table 28, below, lists the DNA sequences of the designed CpG-reduced or depleted guide scaffolds.

TABLE 28
DNA sequences encoding CpG-reduced
or depleted guide RNA scaffolds

| Scaffold ID | SEQ ID NO |
|---|---|
| 320 | 14065 |
| 321 | 14066 |
| 322 | 14067 |
| 323 | 14068 |
| 324 | 14069 |
| 325 | 14070 |
| 326 | 14071 |
| 327 | 14072 |
| 328 | 14073 |
| 329 | 14074 |
| 330 | 14075 |
| 331 | 14076 |
| 332 | 14077 |
| 333 | 14078 |
| 334 | 14079 |
| 335 | 14080 |
| 336 | 14081 |
| 337 | 14082 |
| 338 | 14083 |
| 339 | 14084 |
| 340 | 14085 |
| 341 | 14086 |

### Generation of CpG-Depleted AAV Plasmids:

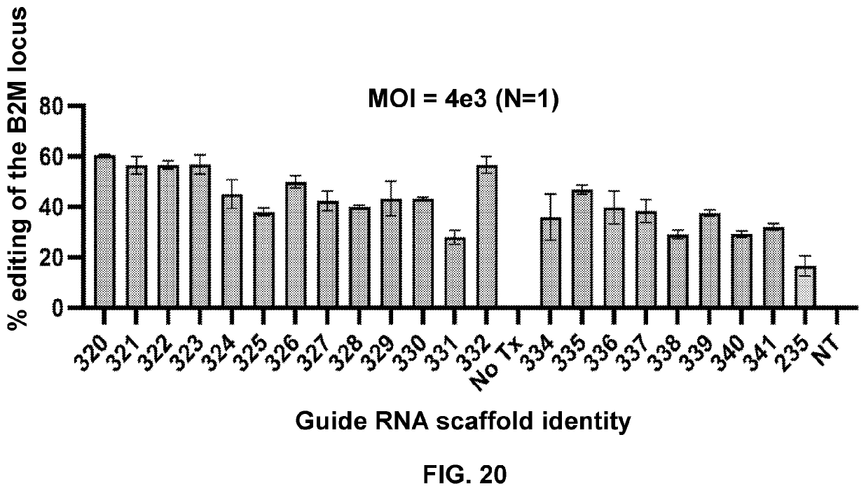

The CpG-reduced or depleted gRNA scaffolds were tested in the context of AAV vectors that were otherwise CpG-depleted, with the exception of the AAV2 ITRs. Specifically, nucleotide substitutions to replace native CpG motifs in AAV components were designed in silico based on homologous nucleotide sequences from related species for the following elements: the murine Ula snRNA (small nuclear RNA) gene promoter, the bGHpA (bovine growth hormone polyadenylation) sequence, and the human U6 promoter. The coding sequence for CasX 491 was codon-optimized for CpG depletion. All resulting sequences (Tables 28 and 29) were ordered as gene fragments with the appropriate overhangs for cloning and isothermal assembly to replace individually the corresponding elements of the existing base AAV plasmid. Spacer 7.37 (GGCCGAGAUGUCUCGCUCCG; SEQ ID NO: 14124), which targets the endogenous B2M gene, was used for the experiments discussed in this example. The first time that the experiment was performed (“N=1”), a sample with the non-targeting spacer 0.0 was also included as a control (CGAGACGTAATTACGTCTCG, SEQ ID NO: 14125; see FIG. 20).

The resulting AAV constructs were generated using standard molecular cloning techniques. Cloned and sequence-validated plasmid constructs were midi-prepped for subsequent nucleofection and AAV vector production. The sequences of the additional components of AAV constructs, with the exception of sequences encoding the gRNAs (Table 27), are listed in Table 29.

TABLE 29
Sequences of AAV elements (5′-3′ in AAV construct)

| Element | SEQ ID NO: |
|---|---|
| AAV2 5′ ITR | 13778 |
| CpG-depleted U1a promoter | 14087 |
| CpG-depleted cMycNLS-CasX491- | 14088 |
| cMycNLS | |
| CpG-depleted bGH-polyA sequence | 14089 |
| CpG-depleted U6 promoter | 14090 |

See sgRNA sequences in Table 28, above.

| | |
|---|---|
| AAV2 3′ ITR | 13779 |

### Aav Production:

Suspension-adapted HEK293T cells, maintained in FreeStyle 293 media, were seeded in 20-30 mL of media at 1.5E6 cells/mL on the day of transfection. Endotoxin-free pAAV plasmids with the transgene flanked by ITR repeats were co-transfected with plasmids supplying the adenoviral helper genes for replication and AAV rep/cap genome using PEI Max (Polysciences) in serum-free Opti-MEM media. Three days later, cultures were centrifuged to separate the supernatant from the cell pellet, and the AAV particles were collected, concentrated, and filtered following standard procedures.

To determine the viral genome (vg) titer, 1 μL from crude lysate viruses was digested with DNase and ProtK, followed by quantitative PCR. 5 μL of digested virus was used in a 25 μL qPCR reaction composed of IDT primetime master mix and a set of primer and 6′FAM/Zen/IBFQ probe (IDT) designed to amplify a 62 bp-fragment located in the AAV2-ITR. An AAV ITR plasmid was used as reference standards to calculate the titer (vg/mL) of viral samples.

### AAV Transduction of Induced Neurons In Vitro:

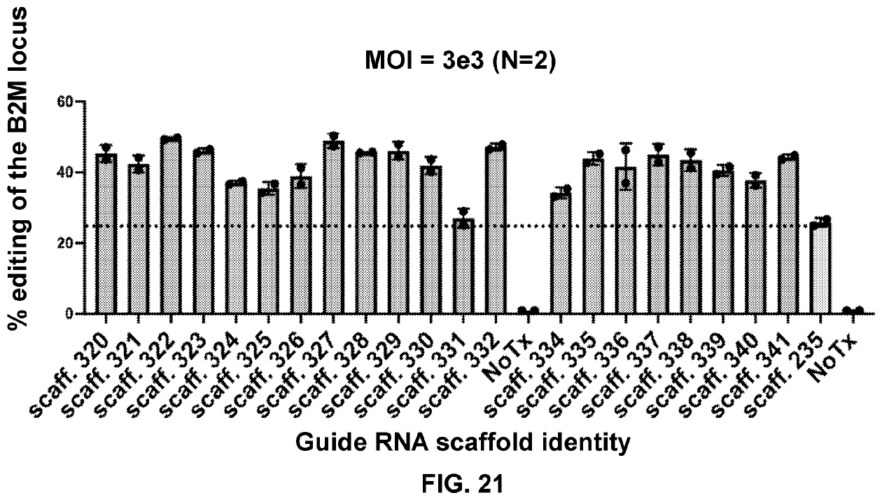

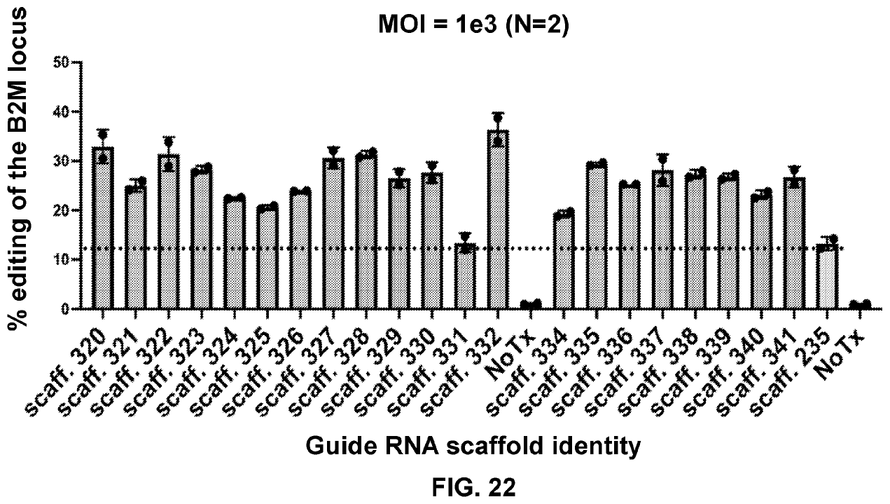

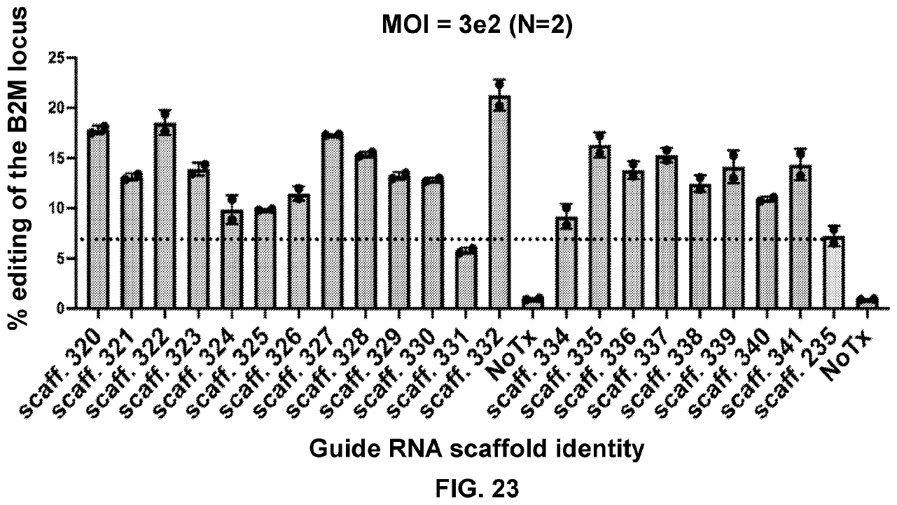

24 hours prior to transduction, 50,000 induced neurons per well were seeded on Matrigel-coated 96-well plates. AAVs expressing the CasX:gRNA system with various versions of the guide scaffold were then diluted in neuronal plating media and added to cells. The first time that the experiment was performed (“N=1”), cells were transduced at a multiplicity of infection (MOI) of 4e3 viral genomes (vg)/cell (see FIG. 20). Seven days post-plating, induced neurons were transduced with virus diluted in fresh feeding media. Eight days post-transduction, cells were lifted using lysis buffer, 4-well replicates were pooled per experimental condition, and genomic DNA (gDNA) was harvested and prepared for editing analysis at the B2M locus using next generation sequencing (NGS). The second time that the experiment was performed (“N=2”), cells were transduced at an MOI of 3e3 vg/cell, 1e3 vg/cell, or 3e2 vg/cell (see FIG. 21, FIG. 22, and FIG. 23). Seven days post-plating, induced neurons were transduced with virus diluted in fresh feeding media. Seven days post-transduction, cells were lifted using lysis buffer, 2-well replicates were pooled per experimental condition, and gDNA was harvested and prepared for editing analysis at the B2M locus using NGS. Samples that were not transduced with AAV were included as controls.

NGS processing and analysis were performed following similar methods previously described in Example 4.

### Results

Mutations were introduced into the guide scaffold 235 in order to reduce the CpG content of the DNA sequence coding the guide scaffold. Surprisingly, compared to scaffold 235, all of the CpG-reduced and CpG-depleted scaffold variants produced higher levels of editing in induced neurons. This was the case with two independent repeats of the experiment (with the results from the first repeat of the experiment shown in FIG. 20, and the results of the second repeat of the experiment shown in FIGS. 21-23), and across multiple MOIs (see FIGS. 20-23). The enhanced level of editing was surprising because the goal of reducing CpG content was to simply preserve editing activity while reducing immunogenicity. Instead, the mutations enhanced editing activity, rather than merely preserving it.

Notably, scaffold 320 showed a significant increase in potency over scaffold 235. Scaffold 320 includes mutations to only two regions of the scaffold; in the pseudoknot stem and the extended stem (regions 1 and 4). Further, some combinations of mutations produced worse editing than scaffold 320. However, even the CpG-reduced scaffolds that performed worse than scaffold 320, such as scaffolds 331 and 334, performed similar to or better than scaffold 235.

Based on these results, without wishing to be bound by theory, it is believed that the boost in potency seen in many of the CpG-reduced and CpG-depleted scaffolds is likely caused by one of the mutations present in all CpG-reduced scaffolds (i.e., region 1 and/or 4). Since the mutation to region 4 is not present in the scaffolds with the extended stem loop replacement (i.e., the third mutation to region 3) and these scaffolds show a similar improvement in potency over 235 as 320 did, it is believed that the beneficial effect is likely caused by the mutation in region 1 (pseudoknot stem), which is present in all of the tested scaffolds. Further experiments will be performed to test the effect of the individual mutations in the pseudoknot stem (region 1) and the extended stem (region 4) separately.

Further, the N=1 data as presented in FIG. 20 indicate that all the new scaffolds carrying the mutation in region 2 (scaffold stem) edited at a slightly lower level than their respective counterparts without this mutation. This suggests that mutating this position in the scaffold stem may have a small deleterious effect on editing potency. This will be examined in additional experiments.

The results described here demonstrate that introducing mutations that reduced the CpG content of the DNA encoding the guide RNA scaffold resulted in improvements in gene editing relative to guide scaffold 235.

Example 12: Formulation of Lipid Nanoparticles (LNPs) to Deliver CasX mRNA and gRNA Payloads to Target Liver Cells

Experiments were performed to encapsulate CasX mRNA and gRNA into LNPs for delivery to target liver cells and tissue. Here, CasX mRNA and gRNA were encapsulated into LNPs using Gen Voy-ILM™ lipids on the Precision NanoSystems Inc. (PNI) Ignite™ Benchtop System and following the manufacturer's guidelines. GenVoy-ILM™ lipids are manufactured by PNI, with a proprietary composition of ionizable lipid:DSPC:cholesterol:stabilizer at 50:10:37.5:2.5 mol %.

Briefly, to formulate LNPs, equal mass ratios of CasX mRNA and gRNA were diluted in PNI Formulation Buffer, pH 4.0. Gen Voy-ILM™ was diluted 1:1 in anhydrous ethanol. mRNA/gRNA co-formulations were performed using a predetermined N/P ratio. The RNA and lipids were run through a PNI laminar flow cartridge at a predetermined flow rate ratio (RNA:Genvoy-ILM™) on the PNI Ignite™ Benchtop System. After formulation, the LNPs were diluted in PBS, pH 7.4, to decrease the ethanol concentration and increase the pH, which increases the stability of the particles. Buffer exchange of the mRNA/sgRNA-LNPs was achieved by overnight dialysis into PBS, pH 7.4, at 4° C. using 10k Slide-A-Lyzer™ Dialysis Cassettes (Thermo Scientific™). Following dialysis, the mRNA/gRNA-LNPs were concentrated to >0.5 mg/mL using 100 kDa Amicon®-Ultra Centrifugal Filters (Millipore) and then filter-sterilized. Formulated LNPs were analyzed on a Stunner (Unchained Labs) to determine their diameter and polydispersity index (PDI). Encapsulation efficiency and RNA concentration was determined by RiboGreen™ assay using Invitrogen's Quant-iT™ Ribogreen™ RNA assay kit. LNPs were used in various experiments as described herein to deliver CasX mRNA and gRNA to target cells and tissue.

### Example 13: In Vivo Editing of the PCSK9 Locus Using LNP

Experiments are performed to demonstrate that the use of the CasX:gRNA system to edit the PCSK9 gene will result in a reduction of circulating PCSK9 when the CasX-related materials are delivered in vivo via LNPs.

### Materials and Methods

### Mouse Lines:

Mouse strains including C57BL/6 inbred strains from Jackson Labs (C57BL/6J) and Charles River Labs (C57BL/6N) are used in experiments to determine editing based on the consensus mouse sequence. Males and females between 4 and 8 weeks of age at the time of injection are used for experimentation.

### LNP:

Lipid nanoparticles (LNPs) are generated using the Precision Nanosystems NanoAssemblr Ignite microfluidics device with GenVoy ionizable lipids. mRNA encoding the CasX constructs 515, 676, and 812 with 5 and 3′ alpha globin UTRs and gRNAs targeting mouse PCSK9 locus with scaffold 316 (guide modification v5) are formulated as described above using a 1:1 mass ratio, utilizing a single mRNA design and a single gRNA construct per formulation. Formulated LNPs are buffer-exchanged to PBS for in vivo injection. At the time of injection, concentrated LNPs are mixed with sterile-filtered PBS and drawn into sterile syringes.

### Injection:

Mice are anesthetized via isoflurane inhalation and anesthesia is verified by loss of righting reflex and toe pinch. Injection of the LNPs is intravenous through the retro-orbital sinus. Mice are observed for five minutes after injection to ensure recovery from anesthesia before being placed into their home cage. Mice are randomized into groups and receive injections of LNPs at doses of 0.3 mg/kg; 1.0 mg/kg; or 3.0 mg/kg. Mice have plasma collected on Day 4 (described below). After 7 days, mice are euthanized for tissue harvest and final plasma collection.

### Tissue Harvest and Editing Outcomes Analysis:

At the time of termination, tissue is collected from injected and naive mice in the experimental cohorts. Following CO2 euthanasia and cervical dislocation, the liver, spleen, heart, and kidney are harvested from each animal. The tissues are quickly dissected from the mouse, washed briefly in PBS, and immersed in Zymo DNA/RNA Shield reagent (Zymo R1100) and frozen at −20° C. until extraction. Genomic DNA and RNA is extracted from the collected tissues after complete lysis using the Zymo Quick DNA/RNA Miniprep kit (Zymo D7001). Isolated DNA and RNA are examined for concentration and purity and stored at −20° C. for DNA and −80° C. for RNA. DNA is subsequently used in PCR reactions to amplify the region targeted by the Stx/gRNA sequence. This amplified region is further isolated and sequenced with NGS technologies to determine formation of insertion/deletion mutations at the targeted locus.

To assess levels of PCSK9 in mouse blood, whole blood is collected from the tail vein into lithium heparin coated tubes (microvettes) on day 4, or via cardiac puncture at termination. These tubes are kept on ice until blood from all animals is collected, and all samples are processed in parallel. The microvette blood collection tubes are centrifuged at 2500×g for eight minutes to separate plasma from the buffy coat and packed erythrocytes. The plasma (avoiding buffy coat and erythrocytes) is aspirated, aliquoted into sterile tubes, and flash frozen on dry ice and is stored at −20° C. until subsequent analysis. The analysis of PCSK9 levels in plasma is performed with an enzyme linked immunosorbent assay (ELISA) kit produced by Abcam (product number ab215538) as an indicator of editing in the liver.

### Example 14: Assessment of Editing by Engineered CasXs 515 and 812 at the Human PCSK9 Locus in HEK293T Cells

Experiments were performed to demonstrate the ability of improved CasX variants 515 and 812 with a targeting gRNA to edit the human PCSK9 locus in HEK293T cells when delivered via transient transfection of a lentiviral plasmid in vitro.

### Materials and Methods

Lentiviral plasmid cloning was performed as previously described in Example 4. Briefly, lentiviral plasmid constructs comprising sequences encoding for CasX variants 515 or 812 with guide scaffold variant 235 and a PCSK9-targeting spacer 6.1, 6.7, 6.8, 6.109, 6.197, 6.200, or 6.203 (sequences listed in Table 14) were generated.

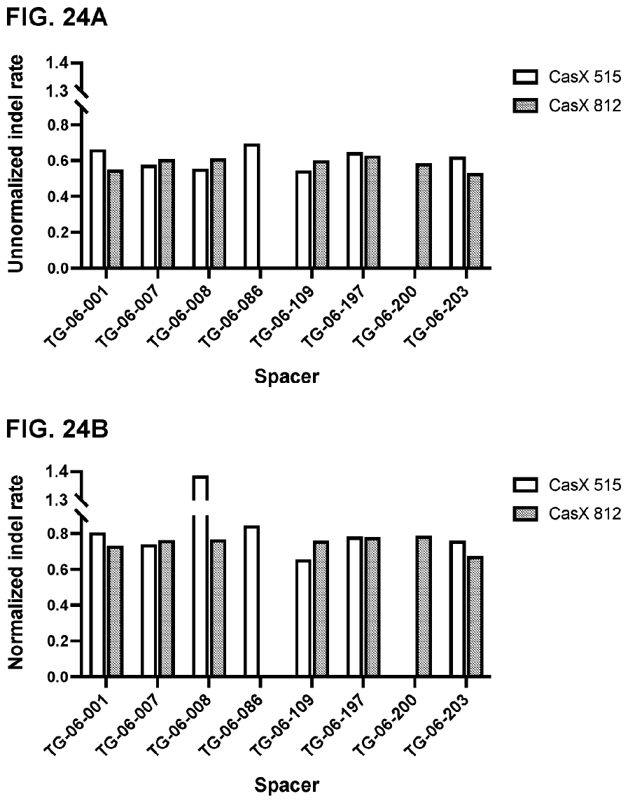

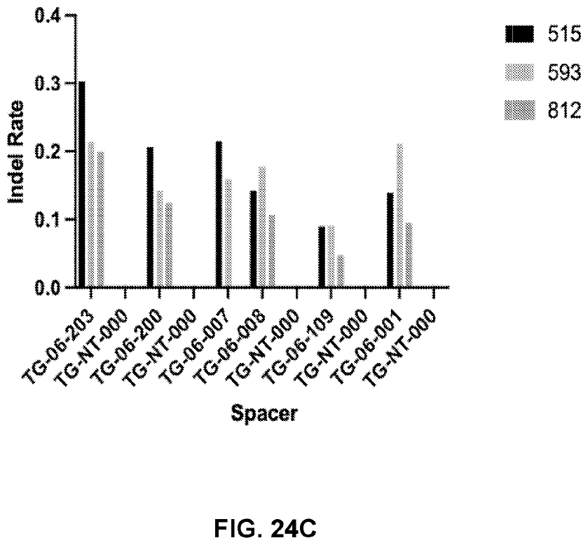

Transient transfection of HEK293T cells and editing assessment by NGS were performed as previously described in Example 4. The data from this editing assessment are shown in FIGS. 24A-24B.

A separate low-dose editing experiment was also subsequently performed. Lentiviral particles containing a transgene that encoded for either CasX 515, 593, or 812, with a PCSK9-targeting gRNA containing scaffold variant 235 and spacers 6.1, 6.109, 6.8, 6.7, 6.200, or 6.203 were produced using standard methods. HEK293T cells were transduced with these lentiviral particles at three doses: 1:1, 1:10, and 1:100 and subjected to puromycin selection two days later. Cell viability was assessed, and cells from the 1:10 dose were allowed to recover for continued culturing. 8 days post-transduction, cells were harvested for gDNA extraction for editing assessment by NGS following methods as described in Example 4. The data from this experiment are shown in FIG. 24C.

### Results

HEK293T cells were transiently transfected with a lentiviral plasmid encoding for a CasX variant (either 515 or 812) and a PCSK9-targeting gRNA with spacer 6.1, 6.7, 6.8, 6.109, 6.197, 6.200, or 6.203 (sequences listed in Table 14). Editing efficiency was assessed by NGS, and the results are plotted in FIGS. 24A-24B. The bar plot in FIG. 24A shows editing levels, measured as indel rate detected by NGS for each individual spacer with either CasX 515 or CasX 812, and the bar plot in FIG. 24B shows the editing levels normalized by transfection efficiency determined using mScarlet expression. The data demonstrate that CasX 515 or 812 and gRNAs containing these spacers were able to edit the human PCSK9 locus, ranging from ~50-70% when unnormalized (FIG. 24A) or ~65-85% when normalized (FIG. 24B). Note that use of CasX 515 with gRNA having spacer 6.8 resulted in a normalized editing rate above 100%, which is likely due to a technical error with the mScarlet expression (FIG. 24B). Notably, within the context of this particular experiment, use of either CasX 515 or 812 resulted in comparable editing levels for each indicated spacers, demonstrating the robustness of using improved CasX variants in targeting the PCSK9 locus in human cells. However, the editing levels observed in FIGS. 24A-24B might have reached saturation levels to discern the editing activity clearly between CasX 515 and CasX 812. As a result, the comparison in activity between CasX 515 and CasX 812 was then repeated in a low-dose editing assessment experiment as described below, as well as in subsequent examples.

A low-dose lentiviral transduction experiment in an attempt to distinguish the editing activity between the CasX 515 and Cas 812 variants further; CasX 593 was also included in this experiment for comparison. The results from this experiment are shown in FIG. 24C. The data demonstrate that across the spacers tested, use of CasX 812 consistently exhibited lower editing activity compared to CasX 515, while comparative activity upon use of CasX 593 varied by spacer. This finding indicates that CasX 812 harbors a relatively lower activity compared to that of CasX 515. Furthermore, in this experiment, use of spacer 203 demonstrated the highest editing rate when paired with CasX 515 or CasX 812 (FIG. 24C).

The results demonstrate that improved CasX variants and PCSK9-targeting gRNAs can effectively edit the human PCSK9 locus with high efficiency in a cell-based assay when delivered by transient transfection or lentiviral transduction.

### Example 15: Assessment of Editing by Engineered CasXs 515, 593, 812 at the Human PCSK9 Locus in Human HepG2 Hepatocytes

Experiments were performed to demonstrate the ability of improved CasX variants 515, 593, and 812 with a targeting gRNA to edit the human PCSK9 locus in human HepG2 hepatocytes when delivered via transient transfection of a lentiviral plasmid in vitro.

### Materials and Methods

### Experiment #1: Use of CasX 515 in HepG2 Cells Delivered Via Transient Transfection

Lentiviral plasmid cloning was performed as previously described in Example 4. Briefly, lentiviral plasmid constructs comprising sequences encoding for CasX variant 515 with guide scaffold variant 235 and a PCSK9-targeting spacer having a TTC PAM were generated. The 81 PCSK9-targeting spacers tested in this experiment are listed in Table 30, with the corresponding spacer sequences listed in Table 14.

TABLE 30
List of 81 PCSK9-targting spacers assessed
in Experiment #1 in this example.
Corresponding sequences are listed in Table 14
Spacer ID

6.1
6.2
6.4
6.5
6.6
6.7
6.8
6.10
6.86
6.87
6.118
6.119
6.120
6.121
6.122
6.123
6.124
6.125
6.126
6.127
6.149
6.150
6.151
6.154
6.155
6.156
6.157
6.160
6.161
6.189
6.190
6.191
6.192
6.193
6.195
6.196
6.197
6.198
6.199
6.200
6.201
6.202
6.203
6.204
6.205
6.206
6.207
6.208
6.209
6.210
6.211
6.212
6.213
6.214
6.215
6.216
6.224
6.225
6.226
6.227
6.228
6.229
6.230
6.231
6.232
6.233
6.234
6.235
6.236
6.237
6.238
6.239
6.240
6.241
6.242
6.243
6.244
6.248
6.249
6.250
6.251
7.37
Non-targeting

### Transient Transfection of HepG2 Cells:

Seeded HepG2 hepatocytes treated with the NATE™ inhibitor were lipofected with a lentiviral vector encoding the CasX variant and gRNA construct. After a media change, cells were harvested three days post-transfection. A subset of harvested cells was used to determine transfection efficiency by measuring mScarlet fluorescence, and the remaining harvested cells were used for editing assessment at the PCSK9 locus by NGS as described in Example 4. The B2M-targeting spacer 7.37 (GGCCGAGAUGUCUCGCUCCG; SEQ ID NO: 14124) served as a positive experimental control, and a non-targeting spacer (NT) served as a negative experimental control. Biological duplicates were performed. The results of averaged editing levels are shown in Table 31.

### Experiment #2: Use of CasX 515, 593, and 812 in HepG2 Cells Delivered Via Transient Transfection

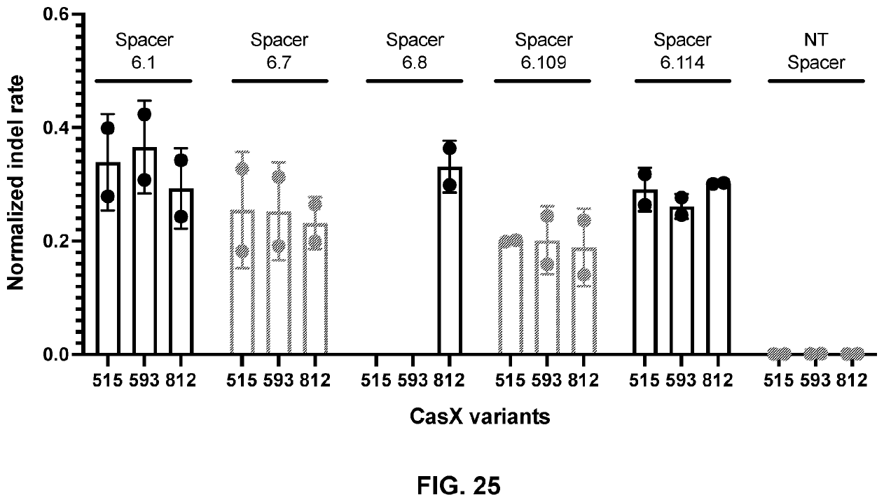

Lentiviral plasmid cloning and transient transfection of HepG2 cells were performed as described previously for Experiment #1. Briefly, lentiviral plasmid constructs comprising sequences encoding for CasX variant 515, 593, or 812 with guide scaffold 235 and a PCSK9-targeting gRNA with spacer 6.1, 6.7, 6.8, 6.109, or 6.114 (sequences listed in Table 14) were generated. These spacers were selected for assessment given their sequence consensus between the human and non-human primate genomes. A non-targeting spacer served as a negative experimental control. The results are shown in FIG. 25.

### Results

Human HepG2 hepatocytes were transiently transfected with a lentiviral plasmid encoding for an improved CasX variant and a PCSK9-targeting gRNA. For Experiment #1, editing activity was assessed for CasX 515 paired with 81 individual PCSK9-targeting gRNAs (spacers listed in Table 30). The resulting editing levels, shown as indel rate normalized by transfection efficiency determined using mScarlet expression, are shown in Table 31. The data demonstrate that CasX 515 and PCSK9-targeting gRNAs were able to edit the human PCSK9 locus in human liver hepatocytes, with an average normalized editing level ranging from ~1-75% (Table 31). Furthermore, the data show that use of spacer 6.1 or 6.86, both of which exhibit sequence conservation between human and non-human primate genomes, resulted in the highest normalized editing rate compared to other NHP-conserved spacers (denoted by open circles in Table 31). Note that the highest average editing rate exhibited by use of spacer 6.203 was discounted given the large variation in editing levels (Table 31). As expected, treatment with CasX 515 and a B2M-targeting spacer resulted in ~50% editing at the B2M locus, while use of a non-targeting spacer did not result in editing (Table 31).

TABLE 31
Normalized editing rate measured for the indicated targeting spacers
as indel rate detected at the PCSK9 locus three days post-transfection.
Data are presented as the average of two biological replicates.
Spacers that exhibit sequence conservation between human and
non-human primate genomes are marked with *

| Spacers | Average indel rate |
|---|---|
| 6.203* | 0.7277 |
| 6.87 | 0.6132 |
| 6.151 | 0.6116 |
| 6.1* | 0.5916 |
| 6.86* | 0.5104 |
| 6.119 | 0.4947 |
| 6.189 | 0.4764 |
| 6.208 | 0.4609 |
| 6.212 | 0.4372 |
| 7.37 | 0.4355 |
| 6.157 | 0.4289 |
| 6.149 | 0.4174 |
| 6.228 | 0.4118 |
| 6.192 | 0.3982 |
| 6.231 | 0.3818 |
| 6.239 | 0.3792 |
| 6.196 | 0.3611 |
| 6.243* | 0.3582 |
| 6.160 | 0.3578 |
| 6.161 | 0.3540 |
| 6.206 | 0.3450 |
| 6.120 | 0.3410 |
| 6.125 | 0.3402 |
| 6.118 | 0.3382 |
| 6.123 | 0.3363 |
| 6.234 | 0.3344 |
| 6.195 | 0.3337 |
| 6.197* | 0.3224 |
| 6.230* | 0.3185 |
| 6.211 | 0.3109 |
| 6.236 | 0.3064 |
| 6.235* | 0.3037 |
| 6.121 | 0.3034 |
| 6.7* | 0.2976 |
| 6.250 | 0.2965 |
| 6.154 | 0.2960 |
| 6.248 | 0.2960 |
| 6.155 | 0.2941 |
| 6.124 | 0.2913 |
| 6.205* | 0.2882 |
| 6.227 | 0.2839 |
| 6.241 | 0.2805 |
| 6.251 | 0.2747 |
| 6.215 | 0.2706 |
| 6.244* | 0.2703 |
| 6.216 | 0.2604 |
| 6.190 | 0.2579 |
| 6.4 | 0.2515 |
| 6.150 | 0.2496 |
| 6.8* | 0.2481 |
| 6.249 | 0.2476 |
| 6.210 | 0.2474 |
| 6.200* | 0.2454 |
| 6.226 | 0.2374 |
| 6.122 | 0.2372 |
| 6.229 | 0.2362 |
| 6.126 | 0.2273 |
| 6.204 | 0.2259 |
| 6.5 | 0.2224 |
| 6.213* | 0.2098 |
| 6.238 | 0.2091 |
| 6.127 | 0.1995 |
| 6.198 | 0.1992 |
| 6.240* | 0.1955 |
| 6.232 | 0.1945 |
| 6.225 | 0.1918 |
| 6.233 | 0.1886 |
| 6.199 | 0.1664 |
| 6.242 | 0.1656 |
| 6.202* | 0.1516 |
| 6.224 | 0.1501 |
| 6.214 | 0.1485 |
| 6.207 | 0.1431 |
| 6.191* | 0.1426 |
| 6.193 | 0.1340 |
| 6.237 | 0.1207 |
| 6.10 | 0.1084 |
| 6.6 | 0.0931 |
| 6.156 | 0.0931 |
| 6.209 | 0.0856 |
| 6.2* | 0.0775 |
| 6.201 | 0.0115 |
| Non-targeting | 0.0000 |

For Experiment #2, the editing activity for CasX 515 was assessed, along with the editing activity of CasX 593 and CasX 812, when paired with a gRNA having spacer 6.1, 6.7, 6.8, 6.109 or 6.114 in HepG2 hepatocytes. The resulting normalized editing levels are shown in the bar plot in FIG. 25. The data demonstrate that CasX 515, 593, and 812 were all able to induce editing at the PCSK9 locus when paired with the indicated spacers (FIG. 25). Use of either spacer 6.1 or 6.114 with any of the three CasX variants or CasX 812 with spacer 6.8 resulted in the similarly highest editing level of ~30%, compared to the editing rate of ~25% with use of spacer 6.7 or ~20% with use of spacer 6.109 (FIG. 25). As expected, treatment with a CasX variant and a non-targeting spacer did not result in editing.

The results demonstrate that improved CasX variants and PCSK9-targeting gRNAs can effectively edit the human PCSK9 locus with high efficiency in human hepatocytes in vitro when delivered by transient transfection.

### Example 16: Assessment of Editing by CasX Variant 491 at the PCSK9 Locus in Non-Human Primate Cells

Experiments were performed to demonstrate the ability of CasX variant 491 with a PCSK9-targeting gRNA to edit the PCSK9 locus in non-human primate cells. Here, the CasX variant and PCSK9-targeting gRNA were packaged and delivered by AAVs in vitro.

### Materials and Methods

### AAV Construct Cloning:

AAV constructs containing a UbC promoter driving CasX expression and a Pol III U6 promoter driving gRNA scaffold 235 with a PCSK9-targeting spacer were generated using standard molecular cloning techniques. Spacer 6.1, 6.109, 6.114, and 6.164 targeting the PCSK9 locus were selected for this experiment because of their sequence consensus between human and the non-human primate species. Cloned and sequence-validated AAV constructs were midi-prepped and subjected to quality assessment prior to transfection into HEK293T cells for AAV production.

AAV production and titering were performed as described in Example 5.

### In Vitro AAV Transduction of Non-Human Primate Fibroblasts:

Two non-human primate fibroblast cell lines were used in this experiment: African green monkey fibroblasts (AGM; ATCC; No. ATCC CCL-81) and cynomolgus macaque fibroblasts (CM; Cell Biologics, No. MK-6019). ~30,000 AGM fibroblasts were seeded per well in a 96-well plate in EMEM media containing 10% FBS; in parallel, ~30,000 CM fibroblasts were seeded per well in a 96-well plate in FB media. 24 hours later, seeded cells were transduced with AAV encoding CasX protein 491 and a gRNA with scaffold 235 and a PCSK9-targeting spacer (6.1, 6.109, 6.114, or 6.164) at two MOIs: 3.33E5 vg/cell and 1.11E5 vg/cell. Fibroblasts were then harvested 96 hours post-transduction for gRNA extraction using the Zymo Quick DNA miniprep Kit following the manufacturer's instructions. gDNA was used as input for editing analysis by NGS. Briefly, target amplicons were amplified from 50 ng of extracted gDNA with a set of primers targeting the PCSK9 locus and processed as described earlier in Example 4. A non-targeting (NT) spacer served as an experimental control.

### Results

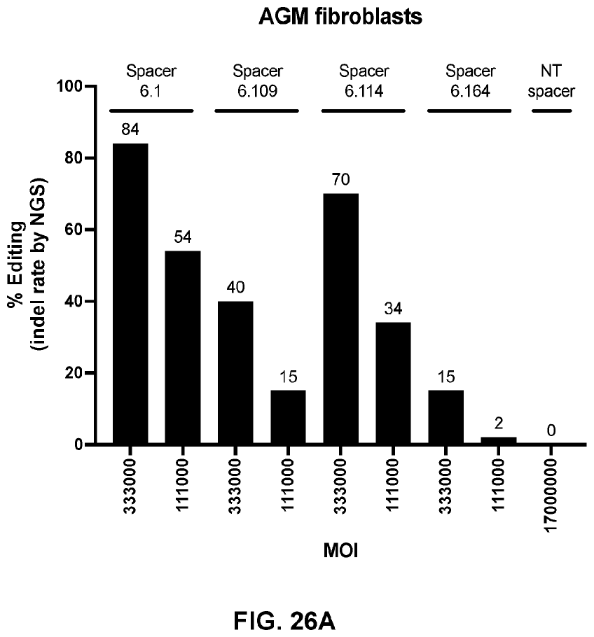

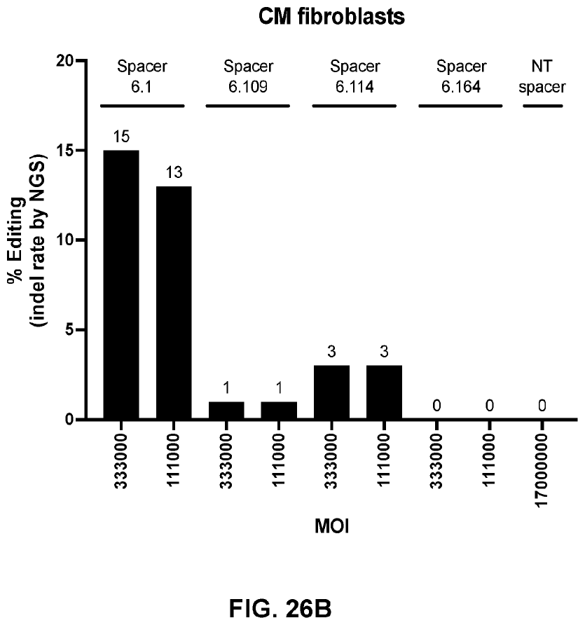

PCSK9-targeting gRNAs containing spacers 6.1, 6.109, 6.114, or 6.164, when paired with CasX variant 491, were assessed for their editing efficiency at the PCSK9 locus when expressed from the AAV episome delivered by AAVs used to transduce two non-human primate fibroblast lines: AGM and CM fibroblasts. The bar plot in FIG. 26A shows the editing levels in transduced AGM fibroblasts, and the bar graph in FIG. 26B shows the corresponding editing results in transduced CM fibroblasts. The data demonstrate that of the spacers evaluated, use of spacer 6.1 resulted in the highest editing level at the PCSK9 locus at both MOIs, achieving nearly 84% editing level at MOI of 3.33E5 vg/cell and ~54% at 1.11E5 vg/cell in AGM fibroblasts (FIG. 26A). Furthermore, use of spacer 6.114 resulted in the second highest editing level, followed by spacer 6.109, and then 6.164. These findings are recapitulated in transduced CM fibroblasts, despite the substantially lower editing efficiency detected, which was attributed to a lower transduction efficiency of the CM line (FIG. 26B).

The results of these experiments demonstrate that CasX and PCSK9-targeting gRNA can effectively edit the PCSK9 locus with high efficiency in different non-human primate cell lines in vitro. These findings also show that AAV delivery of the CasX:gRNA system is feasible to induce editing in non-human primate cells.

### Example 17: Assessment of Editing by Engineered CasXs 515 and 812 at the PCSK9 Locus in Non-Human Primate Hepatocytes

Experiments were performed to demonstrate the ability of engineered CasXs 515 and 812 with a targeting gRNA to edit the PCSK9 locus in non-human primate hepatocytes when delivered via lentiviral transduction in vitro.

### Materials and Methods

### Lentiviral Plasmid Cloning, Lentivirus Production, and Transduction:

Lentiviral plasmid cloning was performed as previously described in Example 4. Briefly, lentiviral plasmid constructs comprising sequences encoding for CasX variant 515 or 812 with guide scaffold variant 235 and a PCSK9-targeting spacer were generated. In one experiment, CasX 515 was assessed with gRNAs having spacer 6.1, 6.8, 6.109, 6.114, or 6.203, and CasX 812 was assessed with gRNAs having spacer 6.1 or 6.109 (spacer sequences listed in Table 14). In a second experiment, CasX 515 was tested with gRNAs having spacer 6.1, 6.86, 6.109, 6.114, 6.197, or 6.203, and CasX 812 was tested with gRNAs having spacer 6.1, 6.8, 6.86, 6.109, 6.114, 6.197, or 6.203 (spacer sequences listed in Table 14). Cloned and sequence-validated constructs were midi-prepped and subjected to quality assessment prior to transfection into HEK293T cells for lentiviral production, as previously described in Example 2.

For lentivirus transduction, primary cynomolgus macaque hepatocytes (CM; BioIVT; M003055-P) were used. ~30,000 CM hepatocytes were seeded per well in a 96-well plate in Williams' E complete medium. 24 hours later, seeded cells were transduced with lentiviral particles containing the CasX:gRNA transgene. For the first experiment, seeded cells were treated with lentiviral particles at two volumetric doses: a low dose of 15 μL and a high dose of 50 μL. For the second experiment, seeded cells were treated with lentiviral particles at five volumetric doses (3.75 μL, 7.50 μL, 15 μL, 30 μL, 60 μL) that were normalized to lentivirus titer.

For both experiments, the following were harvested at six days post-transduction: 1) transduced cells for gDNA extraction for editing assessment at the PCSK9 locus by NGS; 2) media supernatant to measure secreted PCSK9 protein levels by ELISA. For editing analysis by NGS, amplicons were amplified from extracted gDNA with a set of primers targeting the PCSK9 locus and processed as described earlier in Example 4. Secreted PCSK9 levels in the media supernatant were also analyzed using the LEGEND MAX™ Human PCSK9 ELISA kit following the manufacturer's instructions. A non-targeting spacer served as a negative experimental control. ddPCR analysis of lentiviral genomes (vg/dg):

For the second experiment, the number of lentiviral genomes (vg) per diploid genome (dg) was determined in gDNA samples extracted from harvested cells by ddPCR. ddPCR was performed using the Bio-Rad QX200 Droplet Digital PCR instrument according to standard methods and following the manufacturer's protocol and guidelines. Briefly, ddPCR reactions containing the extracted gDNA samples were set up, serially diluted, and subjected to droplet formation using the droplet generator. Within each droplet, a PCR amplification reaction was performed using a primer-probe set specific to the RRE element, an indicator of the lentiviral plasmid, and cynomolgus macaque RPP30, an indicator of the CM genome. Subsequently, droplet fluorescence was determined using the QX200 Droplet Reader with the Bio-Rad QuantaSoft software. To calculate total vg/dg for each sample, the total quantified copy amount for the RRE element was divided by the copy amount calculated for RPP30, and then divided by 2 (diploid genome per cell).

### Results

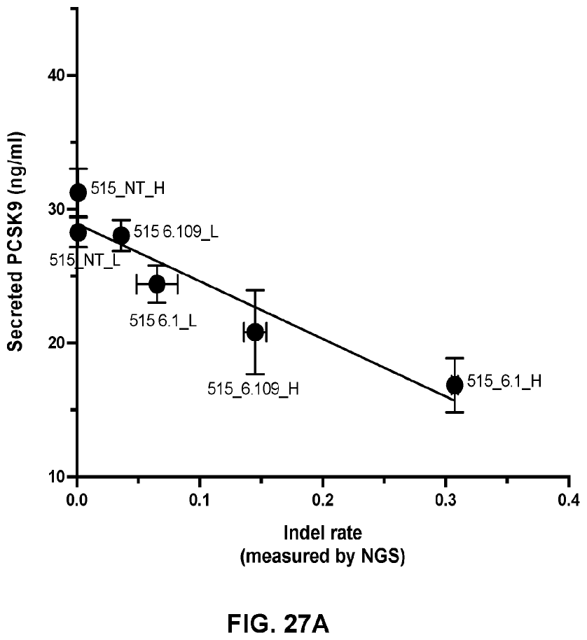

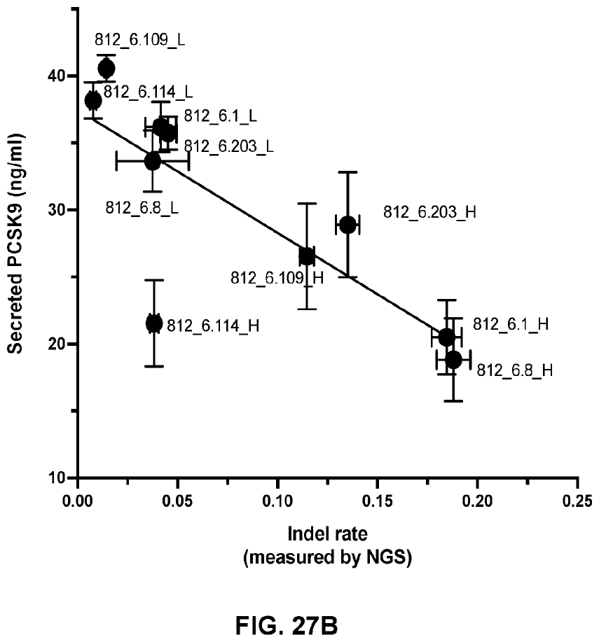

Primary CM hepatocytes were transduced with lentiviral particles containing a transgene that encoded for either CasX 515 or CasX 812, with a PCSK9-targeting gRNA. For the first experiment, CasX 515 was assessed with spacer 6.1, 6.8, 6.109, 6.114, or 6.203, and CasX 812 was assessed with spacer 6.1 or 6.109. The plots in FIGS. 27A and 27B show an inverse correlation between the editing events at the non-human primate PCSK9 locus and the levels of PCSK9 secretion in transduced CM hepatocytes. The data demonstrate that CasX 515 and CasX 812 and PCSK9-targeting gRNAs were able to edit the non-human primate PCSK9 locus in a dose-dependent manner (FIGS. 27A-27B). Furthermore, this dose-dependent effect in editing also resulted in a dose-dependent reduction in PCSK9 secretion (FIGS. 27A-27B). As anticipated, use of the non-targeting spacer with CasX 515 did not induce any editing at the PCSK9 locus.

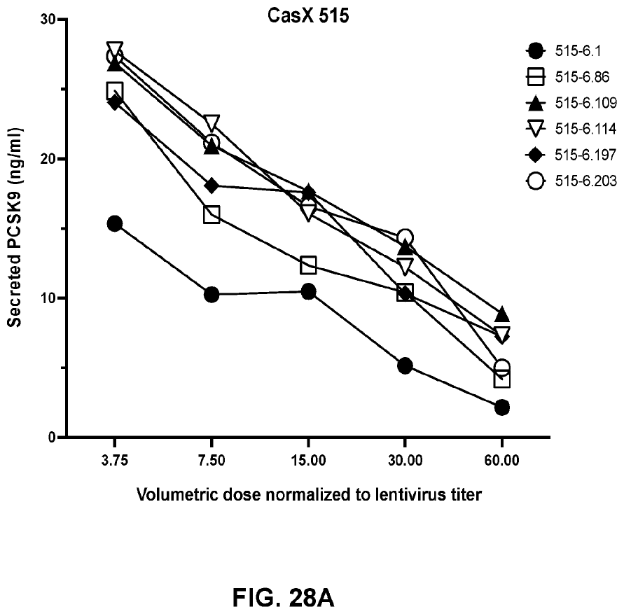

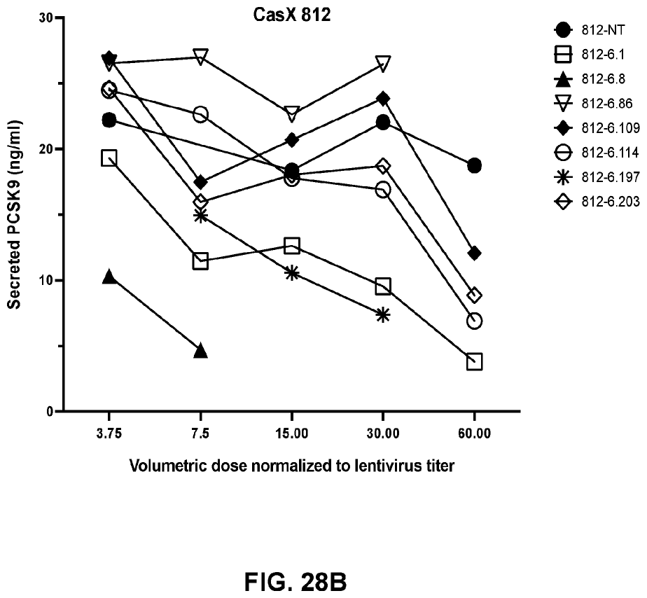

For the second experiment, CasX 515 was evaluated with gRNAs having spacer 6.1, 6.86, 6.109, 6.114, 6.197, or 6.203, and CasX 812 was evaluated with gRNAs having spacer 6.1, 6.8, 6.86, 6.109, 6.114, 6.197, or 6.203 in primary CM hepatocytes. The plots in FIGS. 28A and 28B show the resulting levels of PCSK9 secretion detected in the media supernatant of cultured cells transduced with lentivirus containing a transgene encoding for either CasX 515 or CasX 812 with a PCSK9-targeting gRNA at the indicated titers. The data demonstrate that CasX 515 and CasX 812 and PCSK9-targeting gRNAs were able to reduce PCSK9 secretion in a dose-dependent manner (FIGS. 28A-28B). Notably, use of CasX 515 with targeting gRNA containing spacer 6.1 was most effective in reducing secreted PCSK9 levels in these primary CM hepatocytes (FIG. 28A). Similarly, among the spacers tested with use of CasX 812 that had a complete response curve, use of spacer 6.1 was most efficacious in decreasing secreted PCSK9 levels (FIG. 28B).

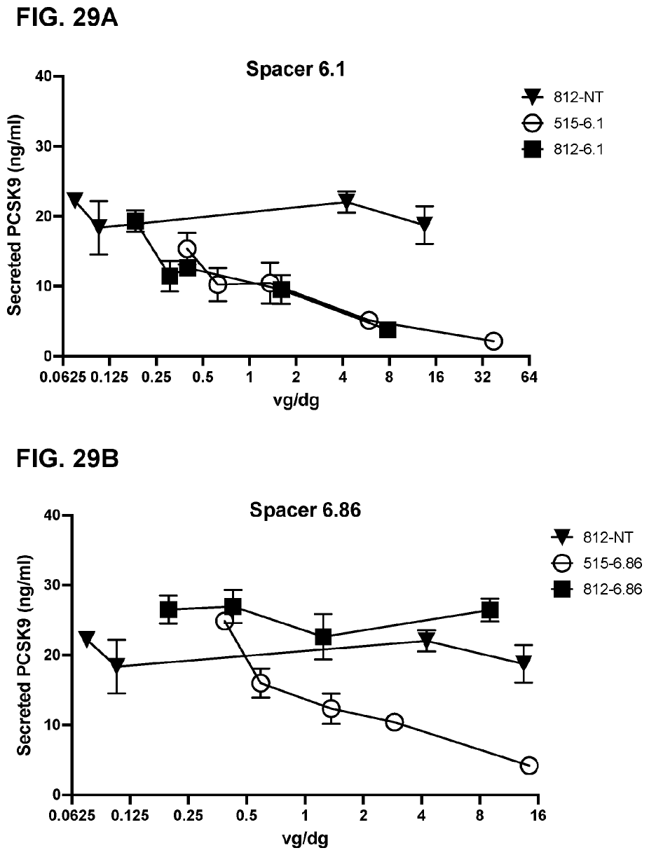

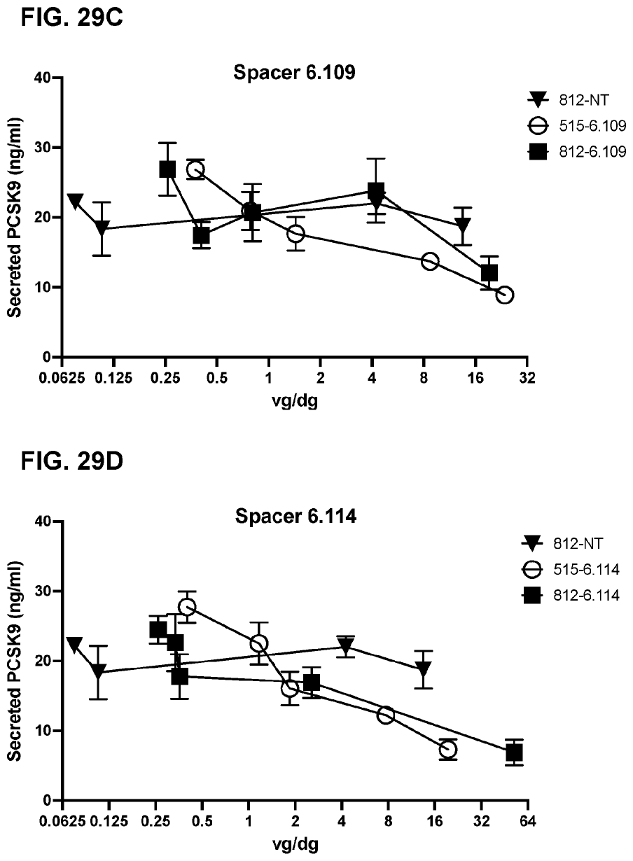

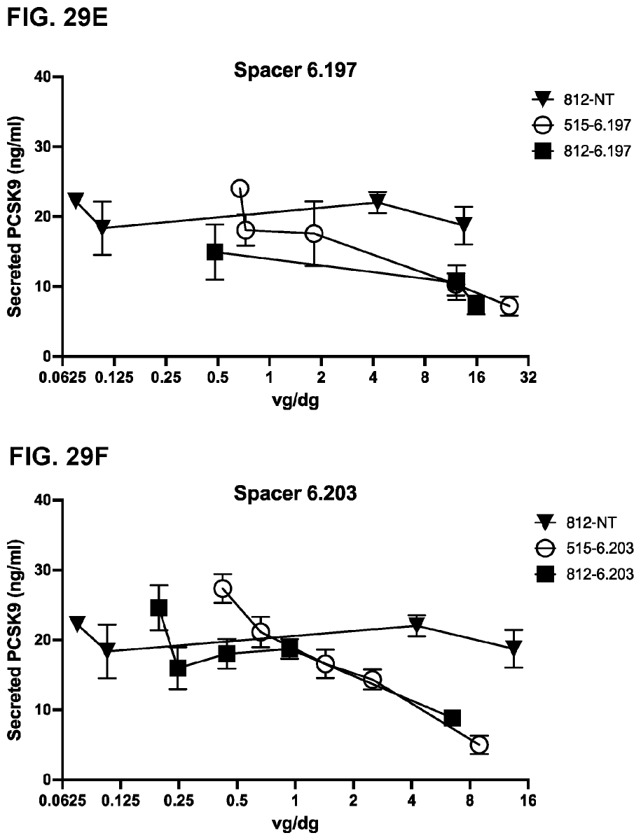

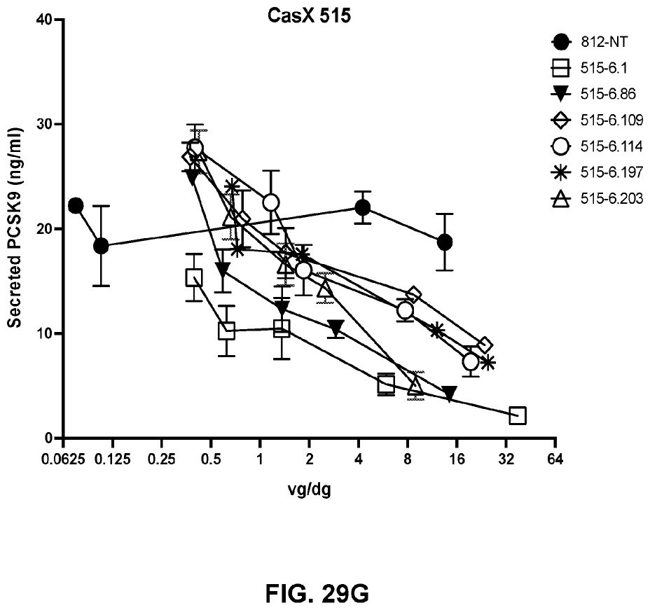

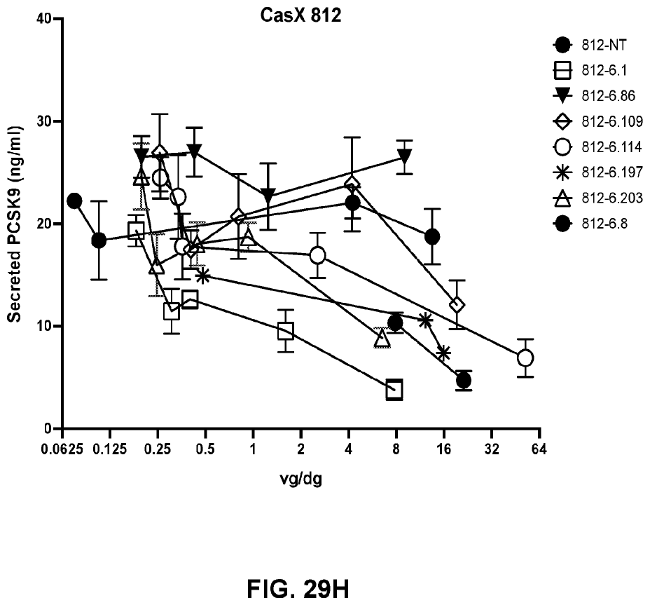

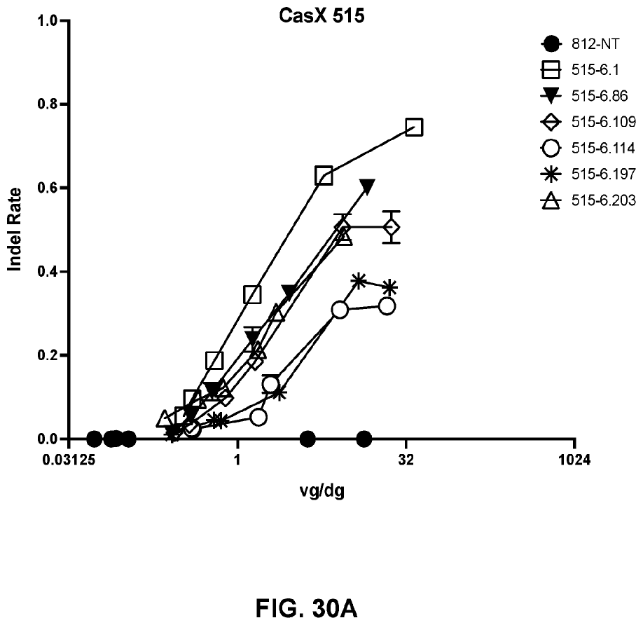

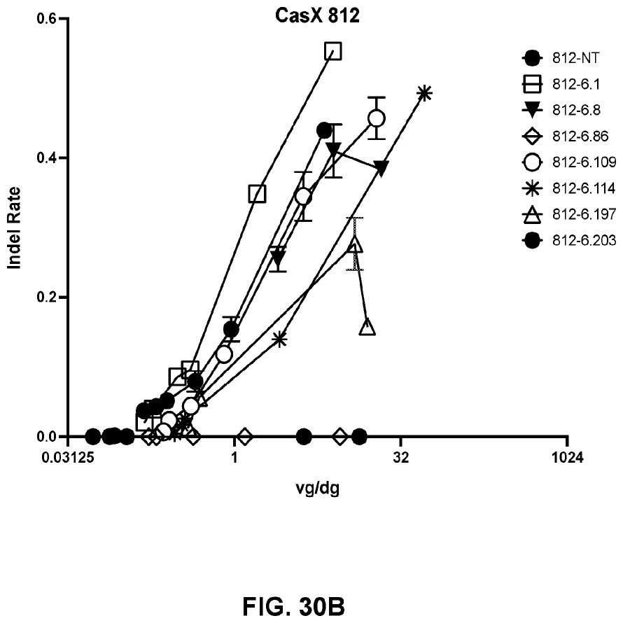

Lentivirus integration within the genome of the primary CM hepatocyte cells was assessed by performing a vg/dg analysis to quantify the number of lentiviral genomes per diploid genome within a given gDNA sample. The vg/dg analysis revealed a positive correlation with LV titer, such that increased vg/dg correlated with increased LV titer (data not shown). Vg/dg was subsequently plotted against secreted PCSK9 levels for each targeting spacer for CasX 515 and CasX 812 (FIGS. 29A-29F). The data demonstrate that there is an overall inverse correlation between LV dose (vg/dg) and resulting PCSK9 secretion level, such that that higher amount of LV delivered correlated with lower PCSK9 secretion. This negative correlation was observed with use of spacers 6.1, 6.109, 6.114, 6.197, or 6.203, and the effects were generally consistent with use of either CasX 515 or CasX 812 for each spacer (FIGS. 29A-29F). Notably, among the spacers tested, use of spacer 6.1 with CasX 515 or CasX 812 appeared to be the most effective in reducing PCSK9 secretion (FIGS. 29G-29H; compare the level of PCSK9 secretion for each spacer to the baseline level of PCSK9 under the NT spacer condition at each vg/dg value). Vg/dg was also plotted against editing activity, measured as indel rate detected at the PCSK9 locus for each targeting spacer for CasX 515 (FIG. 30A) and CasX 812 (FIG. 30B). The data demonstrate that vg/dg positively correlated with indel rate, such that increase in vg per dg corresponded with increased indel rate at the PCSK9 locus (FIGS. 30A-30B), which correlated with decreased PCSK9 secretion (FIGS. 29G-29H). As anticipated, use of a non-targeting spacer with CasX 812 did not result in any detection of indel rate or decreased PCSK9 secretion (FIGS. 29G-29H and 30A-30B).

The results demonstrate that CasX variants and PCSK9-targeting gRNAs can edit the PCSK9 locus in non-human primate primary hepatocytes efficiently in a dose-dependent manner and achieve a corresponding decrease in PCSK9 secretion in a cell-based assay.

Example 18: Assessment of CasX Variants 515, 593, and 812 and gRNAs Using Scaffold Variant 235 with PCSK9-Targeting Spacers to Induce Off-Target Editing

Experiments were performed to assess off-target editing of various CasX proteins and gRNAs with spacers designed to target the human PCSK9 locus. Briefly, CSI-seq, described in Example 10, was conducted to assess several CasX variants individually complexed with a gRNA using scaffold variant 235 with individual human PCSK9-targeting spacers. The nominated off-target sites identified via CSI-seq were subsequently experimentally evaluated and validated.

### Materials and Methods

A CSI-seq experiment was performed as described in Example 10. The off-target editing activity of CasX variants 515, 593, and 812 were each assessed with PCSK9-targeting spacers 6.1, 6.7, 6.8, 6.86, 6.109, 6.114, 6.191, 6.197, 6.200, and 6.203. Spacers sequences are listed in Table 32.

TABLE 32
Sequences of human PCSK9-targeting spacers tested in the CSI-seq experiment.

| | | SEQ ID | | SEQ ID |
|---|---|---|---|---|
| Spacer ID | Spacer DNA sequence | NO: | Spacer RNA sequence | NO: |
| 6.1 | GAGGAGGACGGCCTGGCCGA | 13782 | GAGGAGGACGGCCUGGCCGA | 619 |
| 6.7 | TCCTGGCTTCCTGGTGAAGA | 13788 | UCCUGGCUUCCUGGUGAAGA | 552 |
| 6.8 | TGGCTTCCTGGTGAAGATGA | 13789 | UGGCUUCCUGGUGAAGAUGA | 569 |
| 6.109 | ACAGACAGGTAAGCACGGCC | 13821 | ACAGACAGGUAAGCACGGCC | 644 |
| 6.114 | TCCCAGGCCTGGAGTTTATT | 13825 | UCCCAGGCCUGGAGUUUAUU | 553 |
| 6.191 | TGATAGGGCTGGGCCACTGC | 13854 | UGAUAGGGCUGGGCCACUGC | 571 |
| 6.200 | CTGAGGACCAGCGGGTACTG | 13863 | CUGAGGACCAGCGGGUACUG | 580 |
| 6.203 | CCAGGAGTGGGAAGCGGCGG | 13866 | CCAGGAGUGGGAAGCGGCGG | 583 |

Experimental Validation of Nominated Off-Target Sites Identified from CSI-Seq:

Lentiviral plasmid constructs comprising sequences encoding for a CasX protein (515, 593, or 812), guide scaffold variant 235, and a PCSK9-targeting spacer (listed in Table 32) were generated and validated as described in Example 4.

To validate the nominated off-target sites identified from CSI-seq, HEK293T cells were transfected with a lentiviral plasmid encoding for CasX variant 515, 593, or 812 and a PCSK9-target gRNA using lipofectamine. Two days post-transfection, cells from each experimental sample were harvested for editing analysis by NGS as described in Example 4. A subset of harvested cells was used to determine transfection efficiency by measuring for mScarlet fluorescence as described in Example 4. Briefly, for editing assessment by NGS, amplicons were amplified from 200 ng of extracted gDNA with a set of primers targeting the predicted off-target site identified from CSI-seq, as well as the on-target site at the PCSK9 locus and processed as described earlier in Example 4.

### Results

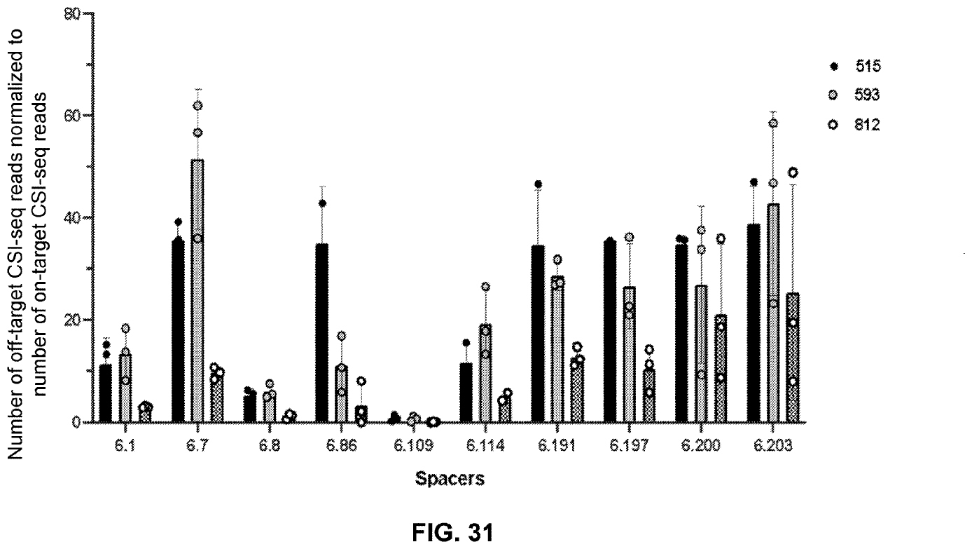

Nominated off-target sites were identified using CSI-seq for each PCSK9-targeting spacer and the indicated CasX variants, and the results were depicted as the normalized level of total off-target CSI-seq reads divided by the total of on-target CSI-seq reads for that targeting spacer (FIG. 31). Spacers with lower percentage of off-target reads over on-target reads were considered to have minimal off-target risk, while spacers with higher normalized level of off-target reads over on-target reads were considered to have a high off-target risk. As illustrated in FIG. 31, the range of level of off-target CSI-seq reads observed for each spacer varied widely. Notably, different CasX variants had distinct off-target profiles, such that, of the three CasX variants, CasX 812 exhibited the lowest off-target risk, indicating that CasX 812 is a CasX variant with relatively increased editing specificity compared to CasX 515 and CasX 593 (FIG. 31). For instance, several spacers had high relative frequencies of off-target reads when paired with CasX 515 or CasX 593, but those off-target rates dropped substantially when paired with CasX 812 (spacers 6.1, 6.7, 6.8, 6.114, 6.191, and 6.197). The data also indicate that spacers 6.1, 6.8, 6.109, and 6.114 appeared to have the lowest off-target risk when paired with either CasX 515 or 812 relative to the remaining spacers tested (FIG. 31).

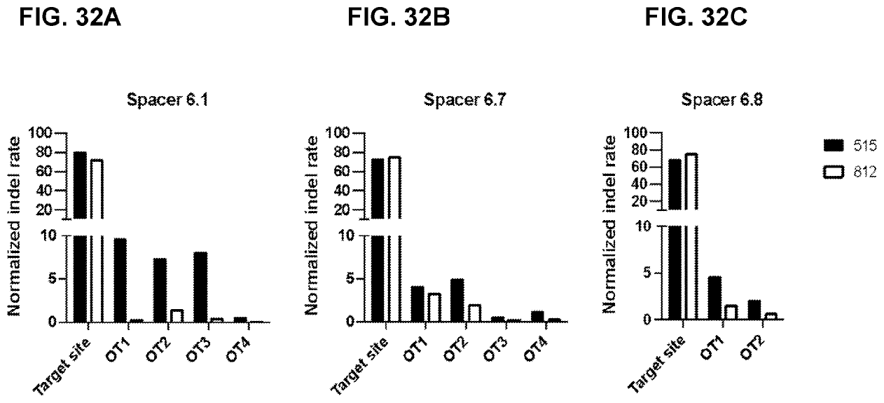

The nominated off-target sites were subsequently experimentally validated. Briefly, HEK293T cells were transfected with a lentiviral plasmid encoding for CasX variant 515, 593, or 812 and a PCSK9-targeting gRNA using lipofectamine. Cells were harvested two days post-transfection for gDNA extraction for editing assessment via NGS to determine the editing level at the predicted off-target sites for spacers 6.1, 6.7, and 6.8. Quantification of percent editing measured as normalized indel rate by NGS for the CSI-seq experiment is illustrated in FIGS. 32A-32C. The indel rate was normalized to the transfection efficiency as determined by reported mScarlet fluorescence. The data demonstrate that in comparison to the on-target indel rate at the intended target site (“Target site”), editing at the individual off-target (“OT”) site for each of the three spacers was relatively noticeable, especially when the spacer was paired with CasX 515. The relative frequency of editing at these respective off-target sites was substantially reduced when the spacer was paired with CasX 812 (FIGS. 32A-32C). Spacers with low-risk off-target profiles and their respective identified off-target sites will be further analyzed in a more therapeutically relevant cell line, such as primary human hepatocytes.

Together, these data demonstrate that multiple CasX variants are capable of editing the PCSK9 locus in human cells in vitro, although with detectable levels of off-target editing. The results further show that CasX variants with higher global specificity can improve off-target profiles of spacers, indicating the importance of robustly assessing specific combinations of spacers and CasX variants to select those with low off-target risk.

Example 19: Demonstration that Altering the UTR Sequences of the Engineered CasX mRNA can Affect CasX-Mediated Editing

5′ and 3′UTRs are essential and required for efficient translation of mRNA. Here, experiments were performed to demonstrate that altering the 5′ and 3′ UTR sequences of the engineered CasX mRNA affects CasX-mediated editing at a target locus when CasX mRNA and targeting gRNAs were delivered in vitro via transfection.

### Materials and Methods

IVT of CasX mRNA:

CasX 676 mRNA was generated by IVT. Briefly, constructs encoding for a 5′UTR region, an optimized CasX 676 with flanking c-MYC NLSes, and a 3′UTR region were cloned into a plasmid containing a T7 promoter and 80-nucleotide poly(A) tail. The coding sequence for CasX 676 was optimized for improved protein expression. The resulting plasmid was linearized prior to use for IVT reactions, which were carried out with CleanCap® AG and N1-methyl-pseudouridine. For the 5′ cap, the CleanCap® AG contains a m7G (5′)ppp(5′)mAG structure, where “m7G” denotes N7-methylguanosine, “mA” denotes 2′O-methyladenosine, and (5′)ppp(5′) denotes a 5′ to 5′ triphosphate bridge. An extra guanine nucleotide was incorporated following the CleanCap® AG to enhance transcription initiation, resulting in the incorporation of m7G (5′)ppp(5′)mAGG as the full 5′ cap structure. As discussed previously in Example 7, the substitution of the uridine ribonucleoside to N1-methyl-pseudouridine improves mRNA performance and reduces mRNA immunogenicity.

IVT reactions were subsequently subjected to DNase digestion to remove template DNA and purification using an oligo-dT column. In this example, two configurations of CasX 676 mRNAs were generated for assessment in vitro. The encoding sequences of the two CasX mRNA configurations are detailed in Table 33. Full-length RNA sequences encoding the CasX mRNA with the chemical modifications are listed in Table 34.

TABLE 33
Encoding sequences of the two CasX mRNA molecules assessed in this example*

| CasX mRNA ID | Component (ID) | Description | DNA sequence or |
|---|---|---|---|
| | | | SEQ ID NO: |
| CasX 676 mRNA #1 | 5'UTR | Human HBA | 14045 |
| | START codon + c- | | 14039 |
| | MYC NLS | | |
| | CasX 676 | | 14040 |
| | c-MYC NLS + STOP | | 14041 |
| | codons | | |
| | 3'UTR | Human HBA | 14046 |
| | Poly(A) tail | | 14044 |
| CasX 676 mRNA #2 | 5'UTR | Synthetic (TriLink) | 14038 |
| | START codon + c- | | 14039 |
| | MYC NLS | | |
| | CasX 676 | | 14040 |
| | c-MYC NLS + STOP | | 14041 |
| | codons | | |
| | 3'UTR | Mouse HBA | 14042 |
| | Xbal restriction site | | TCTAG |
| | (partial) | | |
| | Poly(A) tail | | 14044 |

*Components are listed in a 5' to 3' order within the constructs

TABLE 34
Full-length RNA sequences of CasX mRNA molecules assessed
in this example. The 5′ cap (m7G(5′)ppp(5′)mAG), discussed
in the example herein, is not shown in the
tableModification ‘mψ’ = N1-methyl-pseudouridine

| | SEQ | |
|---|---|---|
| CasX | ID | |
| mRNA | NO: | RNA Sequence |
| CasX | 14091 | ACmψCmψmψCmψGGmψCCCCACAGACmψCAGAGAGAACCCGCCACCAmψGGCCCCmψGCm |
| 676 | | ψGCCAAGAGAGmψGAAGCmψGGAmψAGCAGACAGGAGAmψCAAGCGGAmψmψAAmψAAAA |
| mRNA | | mψmψCGGAGAAGACmψGGmψGAAGGAmψmψCmψAACACAAAGAAGGCmψGGCAAGACACG |
| #1 | | GGGCCCmψAmψGAAGACACmψGCmψGGmψGAGAGmψGAmψGACACCCGACCmψGAGAGAA |
| | | AGACmψGGAAAACCmψGAGAAAGAAGCCmψGAGAAmψAmψCCCCCAGCCCAmψCAGCAAC |
| | | ACAAGCCGGGCCAACCmψGAAmψAAGCmψGCmψGACCGACmψACACCGAAAmψGAAGAAG |
| | | GCCAmψCCmψGCACGmψGmψAmψmψGGGAAGAGmψmψCCAGAAAGACCCAGmψCGGCCmψ |
| | | GAmψGAGCAGAGmψGGCmψCAGCCmψGCCAGCAAGAAGAmψCGAmψCAGAACAAGCmψGA |
| | | AGCCCGAAAmψGGACGAGAAGGGGAACCmψGACAACCGCCGGCmψmψmψGCCmψGmψAGC |
| | | CAGmψGCGGCCAGCCCCmψGmψmψmψGmψGmψACAAACmψGGAACAGGmψGAGCGAAAAG |
| | | GGCAAGGCmψmψACACGAAmψmψACmψmψCGGCAGAmψGCAACGmψGGCCGAGCACGAGA |
| | | AGCmψGAmψCAAGCmψGGCCCAGCmψGAAGCCmψGAGAAGGAmψAGCGAmψGAGGCAGmψ |
| | | GACAmψAmψmψCCCmψGGGCAAGmψmψCGGACAGCGGGCCCmψGGAmψmψmψmψmψAmψm |
| | | ψCCAmψmψCAmψGmψGACCAAGGAAmψCCACCCACCCCGmψCAAGCCmψCmψmψGCCCAA |
| | | AmψmψGCCGGCAACAGAmψACGCCmψCCAGCCCCGmψGGGCAAGGCCCmψGAGCGACGCC |
| | | mψGmψAmψGGGCACCAmψCGCCAGCmψmψCCmψGmψCmψAAGmψACCAGGACAmψmψAmψ |
| | | CAmψCGAGCACCAGAAGGmψGGmψGAAGGGCAACCAGAAGAGACmψGGAGAGCCmψGCGC |
| | | GAGCmψGGCCGGCAAGGAAAACCmψGGAGmψAmψCCmψAGCGmψGACCCmψGCCmψCCmψ |
| | | CAGCCmψCAmψACAAAGGAGGGCGmψGGAmψGCCmψACAACGAAGmψGAmψCGCCCGGGm |
| | | ψGCGGAmψGmψGGGmψGAACCmψGAAmψCmψGmψGGCAGAAGCmψGAAGCmψGmψCmψAG |
| | | AGACGACGCCAAGCCCCmψGCmψGAGACmψGAAGGGCmψmψCCCCAGCmψmψCCCmψCmψ |
| | | GGmψGGAGAGACAGGCAAAmψGAAGmψGGACmψGGmψGGGACAmψGGmψGmψGmψAACGm |
| | | ψGAAGAAGCmψGAmψCAAmψGAGAAGAAGGAGGACGGCAAAGmψGmψmψCmψGGCAGAAm |
| | | ψCmψGGCCGGCmψACAAGCGmψCAGGAGGCCCmψGCGGCCCmψACCmψGAGCAGCGAGGA |
| | | AGACAGAAAGAAGGGCAAGAAGmψmψCGCCCGGmψAmψCAGCmψGGGGGACCmψGCmψGC |
| | | mψGCACCmψCGAGAAGAAGCACGGCGAAGACmψGGGGGAAGGmψGmψACGAmψGAGGCCm |
| | | ψGGGAGCGGAmψCGAmψAAGAAGGmψGGAGGGCCmψGAGCAAGCACAmψCAAGCmψGGAG |
| | | GAGGAACGGAGAmψCmψGAGGACGCCCAGAGCAAGGCCGCCCmψGACCGACmψGGCmψGA |
| | | GAGCCAAGGCCAGCmψmψCGmψCAmψCGAGGGGCmψGAAGGAGGCCGACAAGGACGAGmψ |
| | | mψCmψGCCGGmψGCGAACmψGAAGCmψGCAGAAGmψGGmψACGGAGAmψCmψGAGAGGCA |
| | | AACCmψmψmψCGCCAmψCGAGGCCGAGAACAGCAmψCCmψGGACAmψCAGCGGCmψmψCA |
| | | GCAAGCAGmψACAACmψGCGCCmψmψmψAmψmψmψGGCAGAAGGACGGAGmψGAAGAAGC |
| | | mψGAACCmψGmψACCmψGAmψCAmψCAACmψAmψmψmψCAAGGGCGGCAAGCmψGAGAmψ |
| | | mψCAAGAAGAmψCAAGCCmψGAAGCCmψmψCGAGGCCAACAGAmψmψCmψACACCGmψGA |
| | | mψmψAACAAGAAAAGCGGAGAGAmψCGmψGCCAAmψGGAAGmψGAACmψmψCAACmψmψC |
| | | GACGACCCmψAACCmψGAmψCAmψCCmψGCCCCmψGGCAmψmψmψGGCAAGCGGCAGGGC |
| | | AGAGAGmψmψCAmψCmψGGAACGACCmψGCmψGmψCmψCmψGGAGACCGGCAGCCmψGAA |
| | | GCmψGGCCAACGGCAGAGmψGAmψCGAGAAGACACmψGmψACAACAGACGAACCAGACAA |
| | | GACGAGCCCGCCCmψGmψmψmψGmψGGCCCmψGACCmψmψCGAGAGAAGAGAGGmψGCmψ |
| | | GGACAGCAGCAAmψAmψCAAGCCmψAmψGAACCmψGAmψCGGCGmψGGACCGGGGCGAGA |
| | | ACAmψCCCmψGCCGmψGAmψCGCCCmψmψACCGACCCCGAGGGAmψGCCCmψCmψGAGCC |
| | | GGmψmψmψAAAGACAGCCmψGGGCAACCCmψACCCACAmψCCmψGAGAAmψmψGGCGAGm |
| | | ψCCmψACAAGGAGAAGCAGAGAACCAmψCCAGGCCAAGAAGGAGGmψGGAGCAGCGGCGG |
| | | GCmψGGCGGCmψACmψCCCGGAAGmψACGCCAGCAAGGCCAAGAACCmψGGCCGACGACA |
| | | mψGGmψmψAGAAAmψACCGCCAGAGACCmψCCmψGmψACmψACGCmψGmψGACCCAGGAC |
| | | GCCAmψGCmψGAmψCmψmψCGAGAACCmψGAGCAGAGGCmψmψCGGCAGACAGGGCAAGA |
| | | GAACCmψmψCAmψGGCCGAGAGACAGmψACACCCGGAmψGGAGGACmψGGCmψGACCGCC |
| | | AAGCmψGGCCmψACGAGGGCCmψGCCCmψCmψAAGACCmψACCmψGmψCCAAGACCmψmψ |
| | | GGCACAGmψACACCAGCAAGACAmψGCmψCmψAACmψGCGGCmψmψCACAAmψCACGAGC |
| | | GCCGACmψACGACCGGGmψGCmψGGAGAAACmψGAAGAAGACCGCCACAGGCmψGGAmψG |
| | | ACCACCAmψmψAACGGCAAGGAGCmψGAAGGmψGGAGGGCCAGAmψCACCmψACmψACAA |
| | | CAGGmψACAAACGGCAGAACGmψGGmψGAAGGACCmψGAGCGmψGGAACmψGGAmψAGAC |
| | | mψGAGCGAGGAAAGCGmψAAACAAmψGACAmψCAGCAGCmψGGACCAAGGGCCGGAGCGG |
| | | CGAGGCCCmψGAGCCmψGCmψGAAGAAGAGAmψmψCmψCCCACAGACCAGmψGCAGGAGA |
| | | AGmψmψCGmψGmψGmψCmψGAACmψGCGGCmψmψCGAGACCCACGCCGACGAGCAAGCCG |
| | | CCCmψGAACAmψCGCCCGGmψCmψmψGGCmψmψmψmψCCmψGCGGAGCCAGGAGmψACAA |
| | | GAAGmψACCAGACAAACAAGACCACAGGCAACACAGACAAGAGAGCCmψmψCGmψCGAGA |
| | | CCmψGGCAGAGCmψmψCmψACAGAAAGAAGCmψGAAGGAGGmψGmψGGAAGCCmψGCCGm |
| | | ψGGGAAGCCCCGCmψGCCAAGAGAGmψGAAGCmψGGACmψAAmψAGAmψAAGCmψGGAGC |
| | | CmψCGGmψGGCCAmψGCmψmψCmψmψGCCCCmψmψGGGCCmψCCCCCCAGCCCCmψCCmψ |
| | | CCCCmψmψCCmψGCACCCGmψACCCCCGmψGGmψCmψmψmψGAAmψAAAGmψCmψGAGmψ |
| | | GGGCGGCAAAAAAAAAAAAAAAAAAAAAAAAAAAAAAAAAAAAAAAAAAAAAAAAAAAAA |
| | | AAAAAAAAAAAAAAAAAAAAAAAAAAA |
| CasX | 14092 | AAAmψAAGAGAGAAAAGAAGAGmψAAGAAGAAAmψAmψAAGAGCCACCAmψGGCCCCmψG |
| 676 | | CmψGCCAAGAGAGmψGAAGCmψGGAmψAGCAGACAGGAGAmψCAAGCGGAmψmψAAmψAA |
| mRNA | | AAmψmψCGGAGAAGACmψGGmψGAAGGAmψmψCmψAACACAAAGAAGGCmψGGCAAGACA |
| #2 | | CGGGGCCCmψAmψGAAGACACmψGCmψGGmψGAGAGmψGAmψGACACCCGACCmψGAGAG |
| | | AAAGACmψGGAAAACCmψGAGAAAGAAGCCmψGAGAAmψAmψCCCCCAGCCCAmψCAGCA |
| | | ACACAAGCCGGGCCAACCmψGAAmψAAGCmψGCmψGACCGACmψACACCGAAAmψGAAGA |
| | | AGGCCAmψCCmψGCACGmψGmψAmψmψGGGAAGAGmψmψCCAGAAAGACCCAGmψCGGCC |
| | | mψGAmψGAGCAGAGmψGGCmψCAGCCmψGCCAGCAAGAAGAmψCGAmψCAGAACAAGCmψ |
| | | GAAGCCCGAAAmψGGACGAGAAGGGGAACCmψGACAACCGCCGGCmψmψmψGCCmψGmψA |
| | | GCCAGmψGCGGCCAGCCCCmψGmψmψmψGmψGmψACAAACmψGGAACAGGmψGAGCGAAA |
| | | AGGGCAAGGCmψmψACACGAAmψmψACmψmψCGGCAGAmψGCAACGmψGGCCGAGCACGA |
| | | GAAGCmψGAmψCAAGCmψGGCCCAGCmψGAAGCCmψGAGAAGGAmψAGCGAmψGAGGCAG |
| | | mψGACAmψAmψmψCCCmψGGGCAAGmψmψCGGACAGCGGGCCCmψGGAmψmψmψmψmψAm |
| | | ψmψCCAmψmψCAmψGmψGACCAAGGAAmψCCACCCACCCCGmψCAAGCCmψCmψmψGCCC |
| | | AAAmψmψGCCGGCAACAGAmψACGCCmψCCAGCCCCGmψGGGCAAGGCCCmψGAGCGACG |
| | | CCmψGmψAmψGGGCACCAmψCGCCAGCmψmψCCmψGmψCmψAAGmψACCAGGACAmψmψA |
| | | mψCAmψCGAGCACCAGAAGGmψGGmψGAAGGGCAACCAGAAGAGACmψGGAGAGCCmψGC |
| | | GCGAGCmψGGCCGGCAAGGAAAACCmψGGAGmψAmψCCmψAGCGmψGACCCmψGCCmψCC |
| | | mψCAGCCmψCAmψACAAAGGAGGGCGmψGGAmψGCCmψACAACGAAGmψGAmψCGCCCGG |
| | | GmψGCGGAmψGmψGGGmψGAACCmψGAAmψCmψGmψGGCAGAAGCmψGAAGCmψGmψCmψ |
| | | AGAGACGACGCCAAGCCCCmψGCmψGAGACmψGAAGGGCmψmψCCCCAGCmψmψCCCmψC |
| | | mψGGmψGGAGAGACAGGCAAAmψGAAGmψGGACmψGGmψGGGACAmψGGmψGmψGmψAAC |
| | | GmψGAAGAAGCmψGAmψCAAmψGAGAAGAAGGAGGACGGCAAAGmψGmψmψCmψGGCAGA |
| | | AmψCmψGGCCGGCmψACAAGCGmψCAGGAGGCCCmψGCGGCCCmψACCmψGAGCAGCGAG |
| | | GAAGACAGAAAGAAGGGCAAGAAGmψmψCGCCCGGmψAmψCAGCmψGGGGGACCmψGCmψ |
| | | GCmψGCACCmψCGAGAAGAAGCACGGCGAAGACmψGGGGGAAGGmψGmψACGAmψGAGGC |
| | | CmψGGGAGCGGAmψCGAmψAAGAAGGmψGGAGGGCCmψGAGCAAGCACAmψCAAGCmψGG |
| | | AGGAGGAACGGAGAmψCmψGAGGACGCCCAGAGCAAGGCCGCCCmψGACCGACmψGGCmψ |
| | | GAGAGCCAAGGCCAGCmψmψCGmψCAmψCGAGGGGCmψGAAGGAGGCCGACAAGGACGAG |
| | | mψmψCmψGCCGGmψGCGAACmψGAAGCmψGCAGAAGmψGGmψACGGAGAmψCmψGAGAGG |
| | | CAAACCmψmψmψCGCCAmψCGAGGCCGAGAACAGCAmψCCmψGGACAmψCAGCGGCmψmψ |
| | | CAGCAAGCAGmψACAACmψGCGCCmψmψmψAmψmψmψGGCAGAAGGACGGAGmψGAAGAA |
| | | GCmψGAACCmψGmψACCmψGAmψCAmψCAACmψAmψmψmψCAAGGGCGGCAAGCmψGAGA |
| | | mψmψCAAGAAGAmψCAAGCCmψGAAGCCmψmψCGAGGCCAACAGAmψmψCmψACACCGmψ |
| | | GAmψmψAACAAGAAAAGCGGAGAGAmψCGmψGCCAAmψGGAAGmψGAACmψmψCAACmψm |
| | | ψCGACGACCCmψAACCmψGAmψCAmψCCmψGCCCCmψGGCAmψmψmψGGCAAGCGGCAGG |
| | | GCAGAGAGmψmψCAmψCmψGGAACGACCmψGCmψGmψCmψCmψGGAGACCGGCAGCCmψG |
| | | AAGCmψGGCCAACGGCAGAGmψGAmψCGAGAAGACACmψGmψACAACAGACGAACCAGAC |
| | | AAGACGAGCCCGCCCmψGmψmψmψGmψGGCCCmψGACCmψmψCGAGAGAAGAGAGGmψGC |
| | | mψGGACAGCAGCAAmψAmψCAAGCCmψAmψGAACCmψGAmψCGGCGmψGGACCGGGGCGA |
| | | GAACAmψCCCmψGCCGmψGAmψCGCCCmψmψACCGACCCCGAGGGAmψGCCCmψCmψGAG |
| | | CCGGmψmψmψAAAGACAGCCmψGGGCAACCCmψACCCACAmψCCmψGAGAAmψmψGGCGA |
| | | GmψCCmψACAAGGAGAAGCAGAGAACCAmψCCAGGCCAAGAAGGAGGmψGGAGCAGCGGC |
| | | GGGCmψGGCGGCmψACmψCCCGGAAGmψACGCCAGCAAGGCCAAGAACCmψGGCCGACGA |
| | | CAmψGGmψmψAGAAAmψACCGCCAGAGACCmψCCmψGmψACmψACGCmψGmψGACCCAGG |
| | | ACGCCAmψGCmψGAmψCmψmψCGAGAACCmψGAGCAGAGGCmψmψCGGCAGACAGGGCAA |
| | | GAGAACCmψmψCAmψGGCCGAGAGACAGmψACACCCGGAmψGGAGGACmψGGCmψGACCG |
| | | CCAAGCmψGGCCmψACGAGGGCCmψGCCCmψCmψAAGACCmψACCmψGmψCCAAGACCmψ |
| | | mψGGCACAGmψACACCAGCAAGACAmψGCmψCmψAACmψGCGGCmψmψCACAAmψCACGA |
| | | GCGCCGACmψACGACCGGGmψGCmψGGAGAAACmψGAAGAAGACCGCCACAGGCmψGGAm |
| | | ψGACCACCAmψmψAACGGCAAGGAGCmψGAAGGmψGGAGGGCCAGAmψCACCmψACmψAC |
| | | AACAGGmψACAAACGGCAGAACGmψGGmψGAAGGACCmψGAGCGmψGGAACmψGGAmψAG |
| | | ACmψGAGCGAGGAAAGCGmψAAACAAmψGACAmψCAGCAGCmψGGACCAAGGGCCGGAGC |
| | | GGCGAGGCCCmψGAGCCmψGCmψGAAGAAGAGAmψmψCmψCCCACAGACCAGmψGCAGGA |
| | | GAAGmψmψCGmψGmψGmψCmψGAACmψGCGGCmψmψCGAGACCCACGCCGACGAGCAAGC |
| | | CGCCCmψGAACAmψCGCCCGGmψCmψmψGGCmψmψmψmψCCmψGCGGAGCCAGGAGmψAC |
| | | AAGAAGmψACCAGACAAACAAGACCACAGGCAACACAGACAAGAGAGCCmψmψCGmψCGA |
| | | GACCmψGGCAGAGCmψmψCmψACAGAAAGAAGCmψGAAGGAGGmψGmψGGAAGCCmψGCC |
| | | GmψGGGAAGCCCCGCmψGCCAAGAGAGmψGAAGCmψGGACmψAAmψAGAmψAAGCmψGCC |
| | | mψmψCmψGCGGGGCmψmψGCCmψmψCmψGGCCAmψGCCCmψmψCmψmψCmψCmψCCCmψm |
| | | ψGCACCmψGmψACCmψCmψmψGGmψCmψmψmψGAAmψAAAGCCmψGAGmψAGGAAGmψCm |
| | | ψAGAAAAAAAAAAAAAAAAAAAAAAAAAAAAAAAAAAAAAAAAAAAAAAAAAAAAAAAAA |
| | | AAAAAAAAAAAAAAAAAAAAAAA |

Synthesis of gRNAs:

In this example, gRNAs targeting the mouse PCSK9 locus were designed using gRNA scaffold 174 with a v1 modification profile (see Example 6) and chemically synthesized. The sequences of the PCSK9-targeting spacers are listed in Table 35.

TABLE 35
Sequences of spacers targeting the mouse PCSK9 locus assayed in this example.

| Spacer ID | Target | RNA sequence | SEQ ID NO: |
|---|---|---|---|
| 27.103 | mouse PCSK9 | UAAUCUCCAUCCUCGUCCUG | 13804 |
| 27.105 | mouse PCSK9 | CCAAGAAGCCAGGGAAGAGG | 14093 |
| 27.106 | mouse PCSK9 | ACAUAUCUUUUAUGACCUCU | 14094 |
| 27.107 | mouse PCSK9 | CUGGCUUCUUGGUGAAGAUG | 14095 |
| 27.108 | mouse PCSK9 | UGGUGAAGAUGAGCAGUGAC | 14096 |
| 27.116 | mouse PCSK9 | GCCGUUGCUCCAAGGUAUGG | 14097 |
| 27.117 | mouse PCSK9 | UUCUUGGGGAUCAGGAGGCC | 14098 |

Transfection of CasX mRNA and gRNA into Mouse Hepa1-6 Cells In Vitro:

Editing at the mouse PCSK9 locus was assessed by delivering in vitro transcribed CasX mRNA (CasX mRNA #1 or CasX mRNA #2; see Table 33) and synthesized gRNAs targeting PCSK9 into Hepa1-6 cells via transfection. Briefly, each well of 20,000 Hepa1-6 cells were lipofected with in vitro transcribed mRNA coding for CasX 676 and a PCSK9-targeting gRNA. After a media change, transfected cells were harvested at 20 hours post-transfection for editing assessment at the PCSK9 locus by NGS as described previously in Example 4. As experimental controls, individual transfections of CasX mRNA #1 and CasX mRNA #2 without gRNAs were performed.

### Results

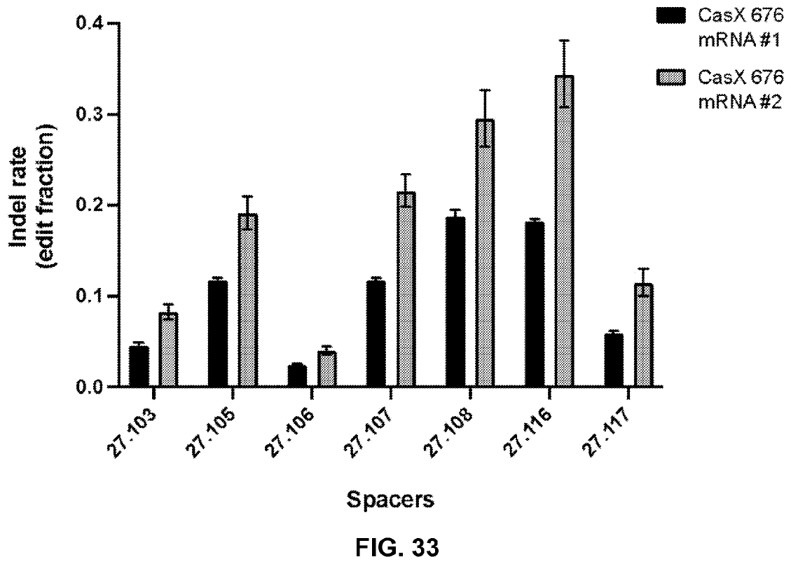

CasX-mediated editing at the mouse PCSK9 locus was used to evaluate the effects of incorporating different 5′ and 3′ UTRs into the engineered CasX mRNA. The plot in FIG. 33 shows the quantification of percent editing measured as indel rate at the PCSK9 locus in mouse Hepa1-6 cells transfected with CasX 676 mRNA #1 or CasX 676 mRNA #2 with the indicated PCSK9-targeting gRNAs. The data demonstrate that for all targeting spacers tested in this experiment, CasX mRNA #2 consistently exhibited higher editing levels at the mouse PCSK9 locus compared to editing levels achieved by CasX mRNA #1. Specifically, the highest level of editing rate achieved was with spacer 27.116, where use of CasX mRNA #2 resulted in ~35% editing efficiency compared to ~20% editing level by CasX mRNA #1 (FIG. 33).

The results demonstrate that altering the 5′UTR and 3′UTR sequences of the CasX mRNA can affect the editing activity of CasX at a target locus in a cell-based assay.

Example 20: Demonstration that Use of Certain PCSK9-Targeting Spacers can Result in Undesired Intracellular PCSK9 Retention

Secretory proteins that cannot properly fold are consequently retained in the endoplasmic reticulum (ER) to be ultimately targeted for proteasomal degradation; however, excessive protein accumulation in the ER could cause ER stress. PCSK9 is initially synthesized as a zymogen (known as pro-PCSK9) that undergoes autocatalytic cleavage during maturation in the ER into an inactive secretory protein (also known as mature or processed PCSK9). Furthermore, certain gain-of-function mutations in the PCSK9 gene resulting in hypercholesterolemia have been shown to be associated with intracellular PCSK9 retention in the ER (Benjannet S et al. NARC-1/PCSK9 and its natural mutants: zymogen cleavage and effects on the low density lipoprotein (LDL) receptor and LDL cholesterol. J. Biol. Chem. 279:48865-48875 (2004); Park S W et al., Post-transcriptional regulation of low density lipoprotein receptor protein by proprotein convertase subtilisin/kexin type 9a in mouse liver. J. Biol. Chem. 279:50630-50638 (2004); Uribe K B et al. A Systematic Approach to Assess the Activity and Classification of PCSK9 Variants. Int. J. Mol. Sci. 22:13602 (2021)), an indication that targeting certain regions of the PCSK9 locus may result in undesired intracellular retention. As a result, experiments were performed to demonstrate that use of certain PCSK9 -targeting spacers can result in unwanted increase in intracellular PCSK9 levels that may possibly cause unexpected consequences such as abnormal ER stress.

### Materials and Methods

In vitro transcription of CasX 676 mRNA #2 (sequence listed in Table 19) was performed as described in Example 6. Guide RNAs using scaffold 316 and a PCSK9-targeting spacer were synthesized with a v1 modification profile (as discussed in Example 6). Spacers 6.1, 6.8, 6.86, 6.114, 6.197, and 6.203 (sequences listed in Table 32) were assessed for intracellular PCSK9 retention.

In Vitro Delivery of CasX mRNA and gRNA Via Transfection:

To determine whether use of certain PCSK9-targeting spacers would result in potential intracellular PCSK9 retention, ~50,000 HepG2 cells were seeded per well in a 96-well plate. CasX 676 mRNA #2 was transfected into HepG2 cells with a PCSK9-targeting gRNA using lipofectamine. After a media change, the following were harvested two days post-transfection: 1) media supernatant to measure secreted PCSK9 protein levels by ELISA; and 2) transfected cells for western blotting analysis to evaluate intracellular PCSK9 levels. Harvested cells were subjected to whole cell lysate extraction for western blotting analysis. Briefly, extracted protein samples were resolved by SDS-PAGE followed by immunoblotting to analyze levels of pro-PCSK9 and processed PCSK9, which were quantified by densitometry. Secreted PCSK9 levels in the media supernatant were analyzed using the BioLegend® ELISA MAX™ kit following the manufacturer's instructions. Naïve, untreated cells and cells transfected with CasX 676 mRNA #2 only served as two experimental controls.

### Results

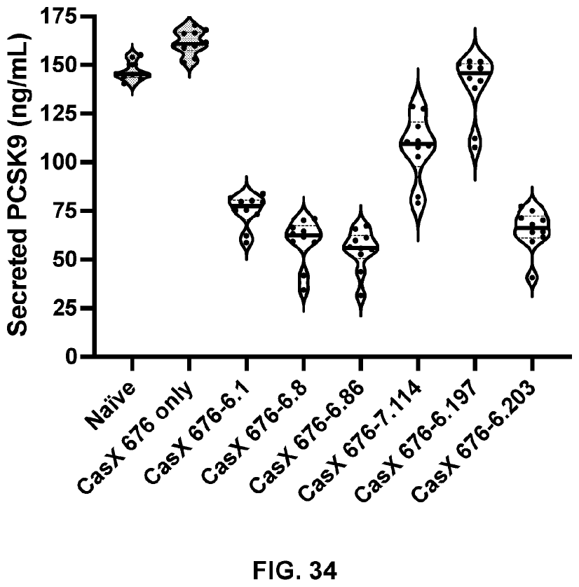

Following transfection of HepG2 cells with CasX 676 mRNA #2 and a PCSK9-targeting gRNA, secreted PCSK9 levels in the media supernatant were quantified by ELISA, and the results are shown in FIG. 34. The data demonstrate that transfection of HepG2s cells with CasX 676 mRNA and the PCSK9-targeting gRNAs resulted in reduced secreted PCSK9 levels to varying degrees. Of the spacers tested, use of spacers 6.1, 6.8, 6.86, and 6.203 resulted in nearly 50% reduction in secreted levels compared to cells transfected with CasX 676 mRNA only (FIG. 34).

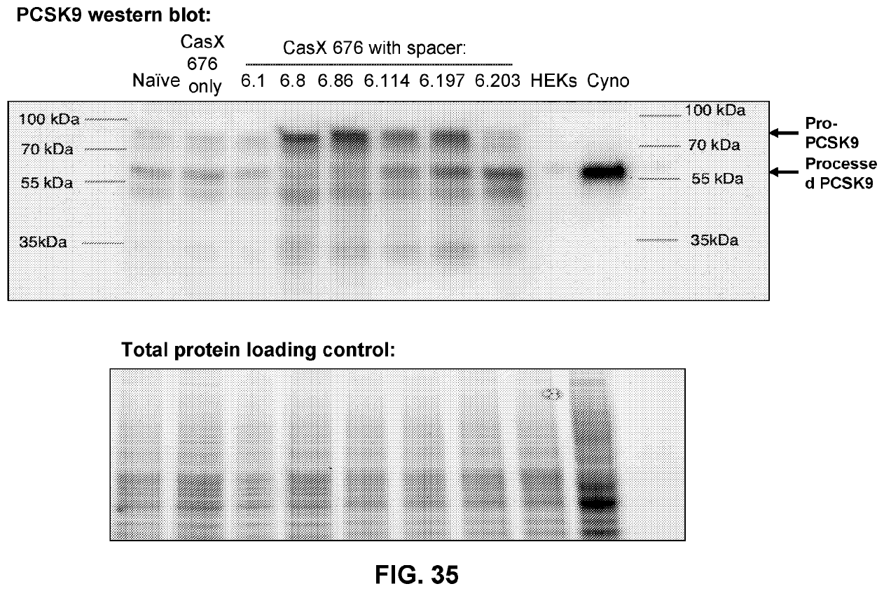

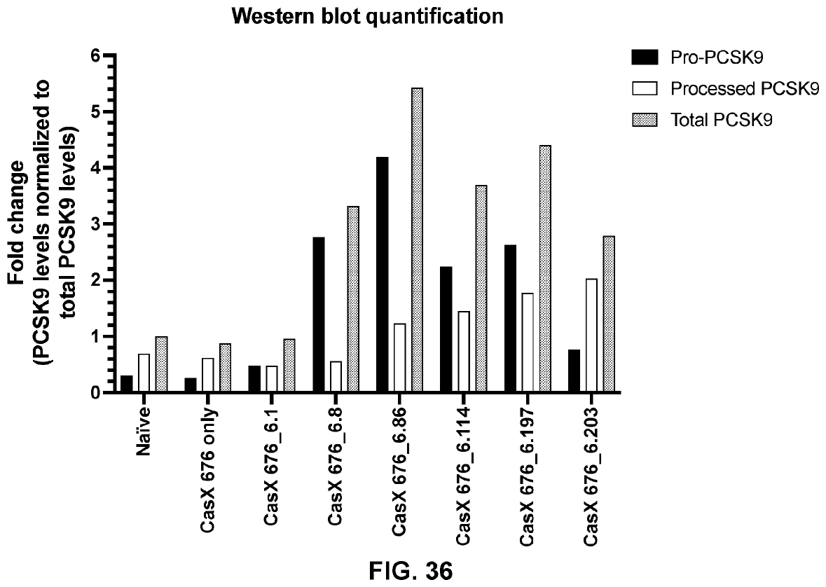

Intracellular levels of PCSK9 were also evaluated in the transfected HepG2 cells. FIG. 35 is a western blot analysis of PCSK9 protein levels, along with the total protein loading control, in the transfected HepG2 cells, and FIG. 36 is a bar plot illustrating the densitometry quantification for pro-PCSK9, processed PCSK9, and total PCSK9 protein levels normalized to total PCSK9 levels from the naïve condition. The data show that of the PCSK9-targeting spacers assessed, only use of spacer 6.1 did not result in substantially increased pro-PCSK9 levels, therefore indicating that use of spacer 6.1 did not increase intracellular protein levels (FIGS. 35-36). While use of spacer 6.203 also did not noticeably increase intracellular protein levels, its use resulted in increased processed PCSK9 levels (FIGS. 35-36), appearing to contradict findings of its effects to reduce secreted PCSK9 levels (FIG. 34) when compared to the either the naïve or CasX mRNA only control. This apparent contradictory effect observed by use of spacer 6.203 indicates that retention of processed PCSK9 may be involved in the mechanism by which use of spacer 6.203 decreases PCSK9 secretion.

The results demonstrate that although use of certain PCSK9-targeting spacers would result in decreased secreted PCSK9 levels, there is a possibility for some of these seemingly effective spacers to exhibit potentially undesired characteristics, such as increased intracellular protein retention. Therefore, the findings from these experiments indicate the use of assessing increased intracellular protein retention as a potential safety criterion to identify effective targeting spacers for therapeutic use.

Example 21: CasX mRNA and PCSK9-Targeting gRNA can be Delivered Via LNPs to Achieve Editing at the Human PCSK9 Locus In Vitro

Experiments were performed to demonstrate that delivery of LNPs encapsulating CasX mRNA and a PCSK9-targeting gRNA can induce editing at the endogenous human PCSK9 locus in primary human hepatocytes. Here, CasX 515 was selected for assessment given its improvement in specificity while maintaining activity compared to the earlier prototype CasX 491, and CasX 812 was selected given its increased specificity.

### Materials and Methods

Generation of CasX mRNAs encoding for CasX 515 and CasX 812 was performed by IVT, following similar methods described earlier in Example 19. Briefly, constructs encoding for a synthetic 5′UTR, an optimized CasX 515 or CasX 812 with flanking c-MYC NLSes, and a 3′UTR derived from the mouse hemoglobin alpha (mHBA) were cloned into a plasmid containing a T7 promoter and 79-nucleotide poly(A) tail. The coding sequences for CasX 515 and CasX 812 were optimized to improve efficient protein expression. The resulting plasmid was linearized prior to use for IVT reactions, which were carried out with CleanCap® AG and N1-methyl-pseudouridine (previously described in Example 19). The DNA sequences encoding the CasX 515 or CasX 812 mRNA molecules are listed in Table 36, with the corresponding mRNA sequences with the chemical modifications listed in Table 37. The protein sequences for CasX 515 and CasX 812 resulting from expression of the IVT mRNA molecules are listed in Table 38.

TABLE 36
Encoding sequences of the two CasX mRNA molecules assessed in this example *.

| CasX mRNA ID | Component (ID) | Description | DNA sequence |
|---|---|---|---|
| | | | or SEQ ID NO: |
| CasX 515 & CasX 812 | 5'UTR | Synthetic (TriLink) | 14038 |
| mRNA | START codon + c-MYC | | 14039 |
| | NLS | | |
| CasX 515 mRNA | CasX 515 | | 14099 |
| CasX 812 mRNA | CasX 812 | | 14100 |
| CasX 515 & CasX 812 | c-MYC NLS + STOP codon | | 14101 |
| mRNA | 3'UTR | Mouse HBA | 14042 |
| | Xbal restriction site (partial) | | TCTAG |
| | Poly(A) tail | | 14102 |

*
Components are listed in a 5' to 3' order within the constructs

TABLE 37
Full-length RNA sequences of CasX mRNA molecules assessed in this example. The
CleanCap ® AG 5′ cap is not shown in the table. Modification ‘mψ’ = N1-methyl-
pseudouridine

| CasX | SEQ | |
|---|---|---|
| mRNA | ID NO: | RNA Sequence |
| CasX 515 | 14103 | AAAmψAAGAGAGAAAAGAAGAGmψAAGAAGAAAmψAmψAAGAGCCACCAmψGGCCCCmψ |
| mRNA | | GCmψGCCAAGAGAGmψGAAGCmψGGAmψAGCAGACAGGAGAmψCAAGCGGAmψmψAAmψ |
| | | AAAAmψmψCGGAGAAGACmψGGmGAAGGAmmψCmψAACACAAAGAAGGCmψGGCAAG |
| | | ACAGGCCCmψAmGAAGACACmψGCmψGGmψGAGAGmψGAmψGACACCCGACCmψGAGA |
| | | GAAAGACmψGGAAAACCmψGAGAAAGAAGCCmψGAGAAmψAmψCCCCCAGCCCAmψCAG |
| | | CAACACAAGCCGGGCCAACCmψGAAmψAAGCmψGCmψGACCGACmψACACCGAAAmψGA |
| | | AGAAGGCCAmψCCmψGCACGmψGmψAmψmψGGGAAGAGmψmψCCAGAAAGACCCAGmψC |
| | | GGCCmψGAmψGAGCAGAGmψGGCmCAGCCmψGCCAGCAAGAAGAmψCGAmψCAGAACA |
| | | AGCmψGAAGCCCGAAAmψGGACGAGAAGGGGAACCmψGACAACCGCCGGCmψmψmψGCC |
| | | mψGmψAGCCAGmψGCGGCCAGCCCCmψGmψmψmψGmψGmψACAAACmψGGAACAGGmψG |
| | | AGCGAAAAGGGCAAGGCmmψACACGAAmψmψACmψmψCGGCAGAmψGCAACGmGGCC |
| | | GAGCACGAGAAGCmGAmψCCmψGCmψGGCCCAGCmψGAAGCCmψGAGAAGGAmψAGCG |
| | | AmψGAGGCAGmψGACAmψAmψmCCCmGGGCAAGmψmψCGGACAGCGGGCCCmψGGAm |
| | | ψmψmψmψmψAmψmψCCAmψmψCAmψGmψGACCAAGGAAmψCCACCCACCCCGmψCAAGC |
| | | CmψCmψmψGCCCAAAmmψGCCGGCAACAGAmψACGCCAGCGGCCCCGmψGGGCAAGGC |
| | | CCmψGAGCGACGCCmψGmψAmψGGGCACCAmψCGCCAGCmψmψCCmψGmψCmψAAGmψA |
| | | CCAGGACAmψmψAmCAmψCGAGCACCAGAAGGmψGGmψGAAGGGCAACCAGAAGAGAC |
| | | mψGGAGAGCCmψGCGCGAGCmψGGCCGGCAAGGAAAACCmψGGAGmψAmψCCmψAGCGm |
| | | ψGACCCmψGCCmψCCmψCAGCCmψCAmψACAAAGGAGGGCGmψGGAmψGCCmψACAACG |
| | | AAGmψGAmψCGCCCGGGmψGCGGAmψGmψGGGmψGAACCmψGAAmψCmψGmψGGCAGAA |
| | | GCmψGAAGCmψGmψCmψAGAGACGACGCCAAGCCCCmψGCmψGAGACmψGAAGGGCmψm |
| | | ψCCCCAGCmψmψCCCmψCmψGGmψGGAGAGACAGGCAAAmψGAAGmψGGACmψGGmψGG |
| | | GACAmGGmψGmψGmψAACGmψGAAGAAGCmψGAmψCAAmψGAGAAGAAGGAGGACGGC |
| | | AAAGmGmψmψCmψGGCAGAAmψCmGGCCGGCmψACAAGCGmψCAGGAGGCCCmψGCG |
| | | GCCCmψACCmψGAGCAGCGAGGAAGACAGAAAGAAGGGCAAGAAGmψmψCGCCCGGmψA |
| | | mψCAGCmψGGGGGACCmψGCmψGCmψGCACCmψCGAGAAGAAGCACGGCGAAGACmUGG |
| | | GGGAAGGmψGmψACGAmψGAGGCCmψGGGAGCGGAmψCGAmψAAGAAGGmψGGAGGGCC |
| | | mψGAGCAAGCACAmψCAAGCmψGGAGGAGGAACGGAGAmψCmψGAGGACGCCCAGAGCA |
| | | AGGCCGCCCmψGACCGACmψGGCmψGAGAGCCAAGGCCAGCmψmψCGmψCAmψCGAGGG |
| | | GCmψGAAGGAGGCCGACAAGGACGAGmψmψCmψGCCGGmψGCGAACmψGAAGCmψGCAG |
| | | AAGmψGGmψACGGAGAmψCmψGAGAGGCAAACCmψmψmψCGCCAmψCGAGGCCGAGAAC |
| | | AGCAmψCCmψGGACAmψCAGCGGCmψmψCAGCAAGCAGmψACAACmψGCGCCmψmψmψA |
| | | mψmψmψGGCAGAAGGACGGAGmψGAAGAAGCmψGAACCmψGmψACCmψGAmψCAmψCAA |
| | | CmψAmψmψmψCAAGGGCGGCAAGCmψGAGAmψCAAGAAGAmψCAAGCCmψGAAGCCm |
| | | ψmψCGAGGCCAACAGAmψmψCmψACACCGmψGAmψmψAACAAGAAAAGCGGAGAGAmψC |
| | | GmψGCCAAmψGGAAGmψGAACmψmψCAACmψmψCGACGACCCmψAACCmψGAmψCAmψC |
| | | CmψGCCCCmψGGCAmψmψmGGCAAGCGGCAGGGCAGAGAGmψmψCAmψCmψGGAACGA |
| | | CCmψGCmψGmψCmψCmψGGAGACCGGCAGCCmψGAAGCmψGGCCAACGGCAGAGmψCAm |
| | | ψCGAGAAGACACmψGmtACAACAGACGAACCAGACAAGACGAGCCCGCCCmψGmψmψmψ |
| | | GmψGGCCCmψGACCmψmψCGAGAGAAGAGAGGmψGCmψGGACAGCAGCAAmψAmψCAAG |
| | | CCmψAmψGAACCmψGAmψCGGCGmψGGACCGGGGCGAGAACAmψCCCmψGCCGmψGAmψ |
| | | CGCCCmψmψACCGACCCCGAGGGAmψGCCCmψCmψGAGCCGGmψmψmψAAAGACAGCCm |
| | | ψGGGCAACCCmψACCCACAmψCCmψGAGAAmψmψGGCGAGmψCCmψACAAGGAGAAGCA |
| | | GAGAACCAmψCCAGGCCAAGAAGGAGGmψGGAGCAGCGGCGGGCmψGGCGGCmψACmψC |
| | | CCGGAAGmψACGCCAGCAAGGCCAAGAACCmψGGCCGACGACAmψGGmψmψAGAAAmψA |
| | | mψmψCGAGAACCmψGAGCAGAGGCmψmψCGGCAGACAGGGCAAGAGAACCmψmψCAmψG |
| | | GCCGAGAGACAGmψACACCCGGAmψGGAGGACmψGGCmψGACCGCCAAGCmψGGCCmψA |
| | | CGAGGGCCmψGCCCmψCmψAAGACCmψACCmψGmψCCAAGACCmψmψGGCACAGmψACA |
| | | CCAGCAAGACAmψGCmψCmψAACmψGCGGCmψmψCACAAmψCACGAGCGCCGACmψACG |
| | | ACCGGGmψGCmψGGAGAAACm@GAAGAAGACCGCCACAGGCmψGGAmψGACCACCAmψm |
| | | ψAACGGCAAGGAGCmψGAAGGmψGGAGGGCCAGAmψCACCmψACmψACAACAGGmψACA |
| | | AACGGCAGAACGmψGGmψGAAGGACCmψGAGCGmψGGAACmψGGAmψAGACmψGAGCGA |
| | | GGAAAGCGmψAAACAAmtGACAmψCAGCAGCmψGGACCAAGGGCCGGAGCGGCGAGGCC |
| | | CmψGAGCCmψGCmψGAAGAAGAGAmψmψCmψCCCACAGACCAGmψGCAGGAGAAGmψmψ |
| | | CGmψGmQGmψCmGAACmψGCGGCmψmψCGAGACCCACGCCGACGAGCAAGCCGCCCmψ |
| | | GAACAmψCGCCCGGmψCmψmψGGCmψmψmψmψCCmψGCGGAGCCAGGAGmψACAAGAAG |
| | | mψACCAGACAAACAAGACCACAGGCAACACAGACAAGAGAGCCmψmψCGmψCGAGACCm |
| | | GGAAGCCCCGCmψGCCAAGAGAGmGAAGCmψGGACmAAGCmψGCCmψmψCmψGCGGG |
| | | GCmψmψGCCmψmψCmψGGCCAmψGCCCmψmψCmψmψCmψCmψCCCmψmψGCACCmψGmψ |
| | | ACCmψCmψmψGGmψCmψmψmψGAAmψAAAGCCmψGAGmψAGGAAGmψCmψAGAAAAAAA |
| | | AAAAAAAAAAAAAAAAAAAAAAAAAAAAAAAAAAAAAAAAAAAAAAAAAAAAAAAAAAA |
| | | AAAAAAAAAAAAA |
| CasX 812 | 14104 | AAAmψAAGAGAGAAAAGAAGAGmψAAGAAGAAAmψAmψAAGAGCCACCAmψGGCCCCmψ |
| mRNA | | GCmψGCCAAGAGAGmψGAAGCmψGGAmψAGCAGACAGGAGAmψCAAGCGGAmψmψAAmψ |
| | | AAAAmψmψCGGAGAAGACmψGGmψGAAGGAmψmψCmψAACACAAAGAAGGCmψGGCAAG |
| | | ACAGGCCCmψAmψGAAGACACmGCmψGGmψGAGAGmψGAmψGACACCCGACCmψGAGA |
| | | GAAAGACmψGGAAAACCmGAGAAAGAAGCCmψGAGAAmψAmψCCCCCAGCCCAmψCAG |
| | | CAACACAAGCCGGGCCAACCmψGAAmψAAGCmψGCmψGACCGACmψACACCGAAAmψGA |
| | | AGAAGGCCAmψCCmψGCACGmψGmψAmψmψGGGAAGAGmψmψCCAGAAAGACCCAGmψC |
| | | GGCCmψGAmψGAGCAGAGmGGCmψCAGCCmψGCCAGCAAGAAGAmψCGAmψCAGAACA |
| | | AGCmψGAAGCCCGAAAmψGGACGAGAAGGGGAACCmψGACAACCGCCGGCmψmmψmψGCC |
| | | mψGmψAGCCAGmψGCGGCCAGCCCCmψGmψmψmψGmψGmψACAAACmψGGAACAGGmψG |
| | | AGCGAAAAGGGCAAGGCmψmψACACGAAmψmψACmψmψCGGCAGAmψGCAACGmψGGCC |
| | | GAGCACGAGAAGCmψGAmCCmψGCmψGGCCCAGCmψGAAGCCmψGAGAAGGAmψAGCG |
| | | AmψGAGGCAGmGACAmψAmψmψCCCmψGGGCAAGmψmψCGGACAGCGGGCCCmψGGAm |
| | | ψmψmψmψmψAmψmψCCAmψmψCAmψGmψGACCAAGGAAmψCCACCCACCCCGmψCAAGC |
| | | CmψCmψmψGCCCAAAmmψGCCGGCAACAGAmψACGCCAGCGGCCCCGmψGGGCAAGGC |
| | | CCmψGAGCGACGCCmψGmψAmψGGGCACCAmψCGCCAGCmψmψCCmψGmψCmψAAGmψA |
| | | CCAGGACAmψmψAmψCAmψCGAGCACCAGAAGGmψGGmψGAAGGGCAACCAGAAGAGAC |
| | | mψGGAGAGCCmψGCGCGAGCmψGGCCGGCAAGGAAAACCmψGGAGmψAmψCCmψAGCGm |
| | | ψGACCCmψGCCmCCmψCAGCCmψCAmψACAAAGGAGGGCGmψGGAmψGCCmψACAACG |
| | | AAGmψGAmψCGCCCGGGmψCGCCAmψGmψGGGmψGAACCmψGAAmψCmψGmψGGCAGAA |
| | | GCmψGAAGCmGmψCmψAGAGACGACGCCAAGCCCCmψGCmψGAGACmψGAAGAAGmψm |
| | | ψCCCCAGCmψmψCCCmψCmψGGmψGGAGAGACAGGCAAAmψGAAGmψGGACmψGGmψGG |
| | | GACAmψGGmψGmψGmψAACGmψGAAGAAGCmψGAmψCAAmψGAGAAGAAGGAGGACGGC |
| | | AAAGmψGmψmCmGGCAGAAmψCmψGGCCGGCmψACAAGCGmψCAGGAGGCCCmψGCG |
| | | GCCCmψACCmψGAGCAGCGAGGAAGACAGAAAGAAGGGCAAGAAGmψmψCGCCCGGmψA |
| | | mψCAGCmψGGGGGACCmψGCmψGCmψGCACCmψCGAGAAGAAGCACGGCGAAGACmψGG |
| | | mψGAGCAAGCACAmψCAAGCmψGGAGGAGGAACGGAGAmψCmψGAGGACGCCCAGAGCA |
| | | AGGCCGCCCmGACCGACmψGGCmψGAGAGCCAAGGCCAGCmψmψCGmCAmψCGAGGG |
| | | GCmψGAAGGAGGCCGACAAGGACGAGmψmψCmψGCCGGmψGCGAACmψGAAGCmGCAG |
| | | AAGmψGGmψACGGAGAmψCmψGAGAGGCAAACCmψmψmψCGCCAmψCGAGGCCGAGAAC |
| | | mψmψmψGGCAGAAGGACGGAGmψGAAGAAGCmψGAACCmψGmψACCmψGAmψCAmψCAA |
| | | CmψAmψmψmψCAAGGGCGGCAAGCmψGAGAmψmψCAAGAAGAmψCAAGCCmψGAAGCCm |
| | | ψmψCGAGGCCAACAGAmψmψCmψACACCGmψGAmψmψAACAAGAAAAGCGGAGAGAmψC |
| | | GmψGCCAAmψGGAAGmψGAACmψmψCAACmψmψCGACGACCCmψAACCmψGAmψCAmψC |
| | | CmψGCCCCmψGGCAmψmψmψGGCAAGCGGCAGGGCAGAGAGmψmψCAmψCmψGGAACGA |
| | | CCmψGCmψGmψCmψCmψGGAGACCGGCAGCCmψGAAGCmψGGCCAACGGCAGAGmψGAm |
| | | ψCGAGAAGACACm@GmVACAACAGACGAACCAGACAAGACGAGCCCGCCCmψGmψmψmψ |
| | | GmψGGCCCmψGACCmψmψCGAGAGAAGAGAGGmψGCmψGGACAGCAGCAAmψAmψCAAG |
| | | CCmψAmψGAACCmψGAmψCGGCGmGGACCGGGGCGAGAACAmψCCCmψGCCGmψGAmψ |
| | | CGCCCmψmψACCGACCCCGAGGGAmψGCCCmψCmψGAGCCGGmψmψmψAAAGACAGCCm |
| | | ψGGGCAACCCmψACCCACAmψCCmψGAGAAmψmψGGCGAGmψCCmψACAAGGAGAAGCA |
| | | GAGAACCAmψCCAGGCCAAGAAGGAGGmψGGAGCAGCGGCGGGCmψGGCGGCmψACmψC |
| | | CCGGAAGmψACGCCAGCAAGGCCAAGAACCmψGGCCGACGACAmψGGmψmψAGAAAmψA |
| | | mψmψCGAGAACCmψGAGCAGAGGCmψmψCGGCAGACAGGGCAAGAGAACCmψmψCAmψG |
| | | GCCGAGAGACAGmψACACCCGGAmψGGAGGACmψGGCmψGACCGCCAAGCmψGGCCmψA |
| | | CCAGCAAGACAmGCmCmAACmψGCGGCmψmψCACAAmψCACGAGCGCCGACmψACG |
| | | ACCGGGmψGCmGGAGAAACmψGAAGAAGACCGCCACAGGCmψGGAmψGACCACCAmψm |
| | | ψAACGGCAAGGAGCmψGAAGGmψGGAGGGCCAGAmψCACCmψACmψACAACAGGmψACA |
| | | AACGGCAGAACGmψGGmψGAAGGACCmψGAGCGmψGGAACmψGGAmψAGACmψGAGCGA |
| | | GGAAAGCGmAAACAAmtGACAmψCAGCAGCmψGGACCAAGGGCCGGAGCGGCGAGGCC |
| | | CmψGAGCCmψGCmψGAAGAAGAGAmψmψCmψCCCACAGACCAGmψGCAGGAGAAGmψmψ |
| | | CGmψGmψGmψCmψGAACmψGCGGCmψmψCGAGACCCACGCCGACGAGCAAGCCGCCCmψ |
| | | GAACAmψCGCCCGGmψCmψmψGGCmψmψmψmψCCmψGCGGAGCCAGGAGmψACAAGAAG |
| | | mψACCAGACAAACAAGACCACAGGCAACACAGACAAGAGAGCCmψmψCGmψCGAGACCm |
| | | ψGGCAGAGCmψmψCmψACAGAAAGAAGCm@GAAGGAGGmψGmψGGAAGCCmψGCCGmψG |
| | | GGAAGCCCCGCmψGCCAAGAGAGmGAAGCmψGGACmψAAGCmψGCCmψmψCmGCGGG |
| | | GCmψmψGCCmψmψCmψGGCCAmψGCCCmψmψCmψmψCmψCmψCCCmψmψGCACCmψGmψ |
| | | ACCmψCmψmψGGmψCmψmψmψGAAmψAAAGCCmψGAGmψAGGAAGmψCmψAGAAAAAAA |
| | | AAAAAAAAAAAAAAAAAAAAAAAAAAAAAAAAAAAAAAAAAAAAAAAAAAAAAAAAAAA |
| | | AAAAAAAAAAAAA |

TABLE 38
Full-length protein sequences of CasX
molecules assessed in this example

| CasX | SEQ ID NO: |
|---|---|
| CasX 515 with flanking c-MYC NLSes | 14105 |
| CasX 812 with flanking c-MYC NLSes | 14106 |

Synthesis of gRNAs:

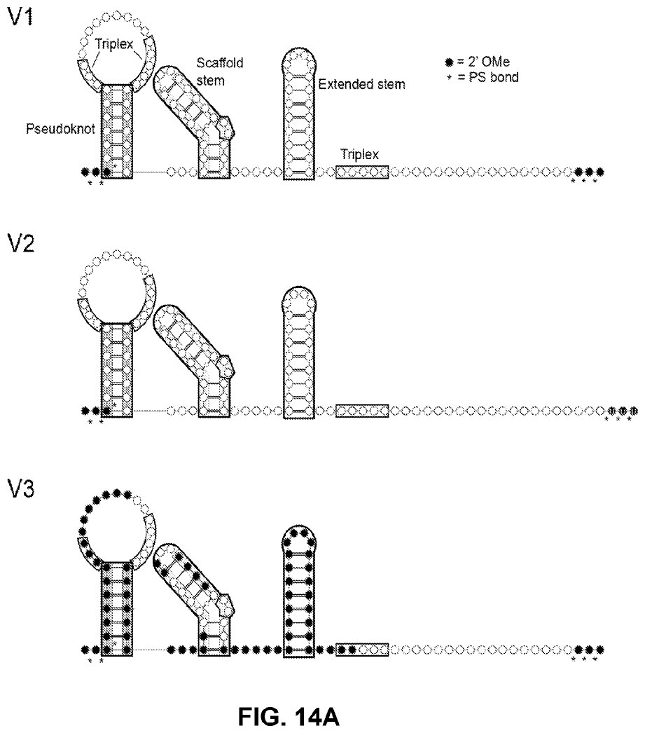

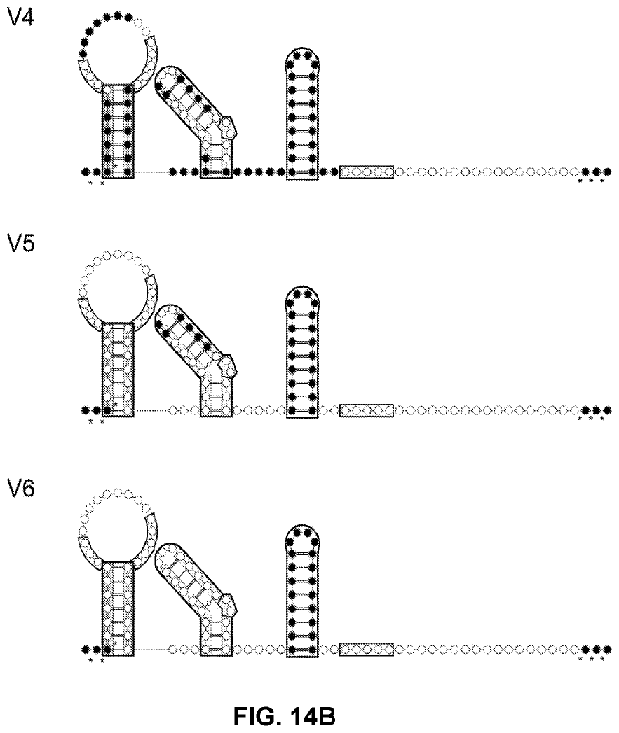

In this example, gRNAs targeting the human PCSK9 locus were designed using gRNA scaffold 316 and chemically synthesized. The sequences of the PCSK9-targeting gRNAs with the v1 modification profile (as described in Example 6) are listed in Table 39. A schematic of the sites of chemical modifications for a ‘v1’ profile of the gRNA scaffold variant 316 is shown in FIG. 14A.

TABLE 39
Sequences of chemically modified gRNAs targeting the human PCSK9 locus assayed
in this example

| gRNA ID (scaffold | | | SEQ ID |
|---|---|---|---|
| variant-spacer) | Target | gRNA sequence | NO: |
| 316-6.1 (v1) | human | mA*mC*mU*GGCGCUUCUAUCUGAUUACUCUGAGCGCCAUC | 14107 |
| | PCSK9 | ACCAGCGACUAUGUCGUAGUGGGUAAAGCUCCCUCUUCGGA | |
| | | GGGAGCAUCAGAGGAGGAGGACGGCCUGGC*mC*mG*mA | |
| 316-6.8 (v1) | human | mA*mC*mU*GGCGCUUCUAUCUGAUUACUCUGAGCGCCAUC | 14108 |
| | PCSK9 | ACCAGCGACUAUGUCGUAGUGGGUAAAGCUCCCUCUUCGGA | |
| | | GGGAGCAUCAGAGUGGCUUCCUGGUGAAGA*mU*mG*mA | |

LNPs using GenVoy-ILM™ lipids were formulated as described in Example 12. Briefly, co-formulations of CasX mRNA and targeting gRNA were performed using a 6:1 N/P ratio.

Delivery of LNPs Encapsulating CasX mRNA and Targeting gRNA into Primary Human Hepatocytes:

Two lots (lot #31 and lot #51) of primary human hepatocytes derived from two different donors (Lonza Biologics), were used in these experiments to assess CasX:gRNA-mediated editing at the human PCSK9 locus when delivered by LNPs. For each lot, ~50,000 cells, cultured in Williams' E media supplemented with FBS, PenStrep, L-glutamine, ITS (insulin, transferrin, sodium selenite), dexamethasone, and Z-VAD-FMK, were seeded per well in a 96-well plate. The next day, seeded cells were treated with varying concentrations of LNPs, which were prepared in five 3-fold serial dilutions starting at 1,200 ng. These LNPs were formulated to encapsulate CasX 515 or CasX 812 mRNA and a PCSK9-targeting gRNA incorporating scaffold variant 316 with either spacer 6.1 or 6.8 (v1; see Table 39). Media was changed two days after LNP treatment, and cells were cultured for three additional days prior to harvesting 1) the media supernatant to measure PCSK9 secretion levels and 2) treated cells for gDNA extraction for editing assessment at the PCSK9 locus by NGS. Briefly, for editing assessment, amplicons were amplified from 200 ng of extracted gDNA with primers targeting the human PCSK9 locus and processed as described in Example 4. PCSK9 secretion levels were measured by ELISA using the BioLegend® ELISA MAX™ kit following the manufacturer's instructions. Treatment with LNPs co-encapsulating a non-targeting gRNA with CasX 515 mRNA served as an experimental control.

### Results

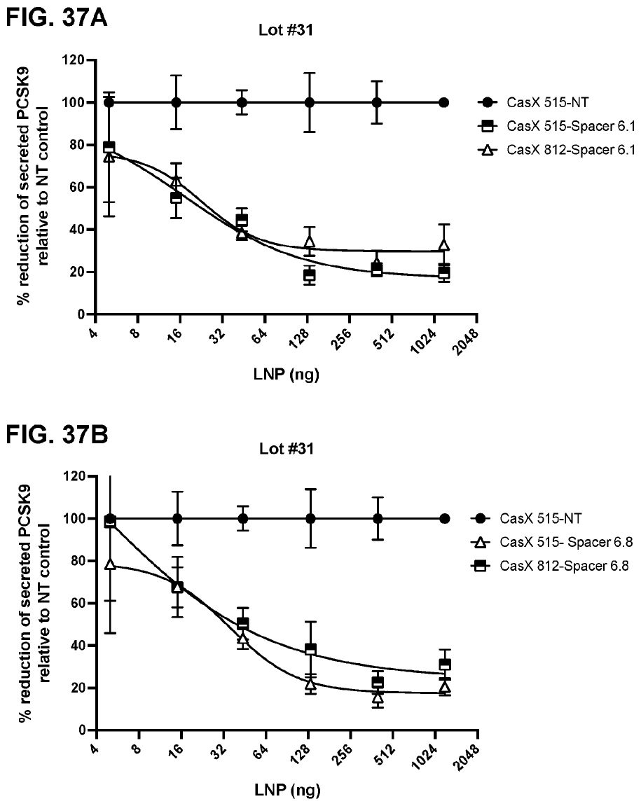

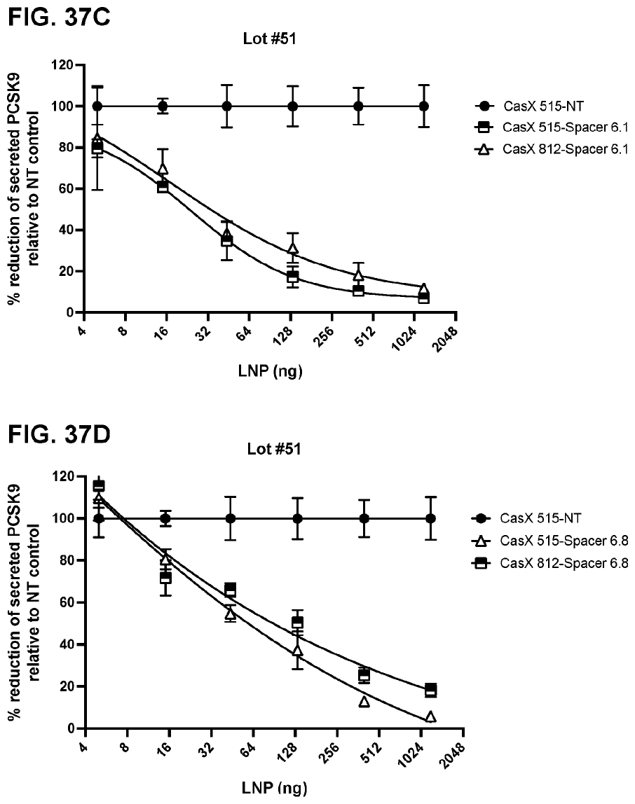

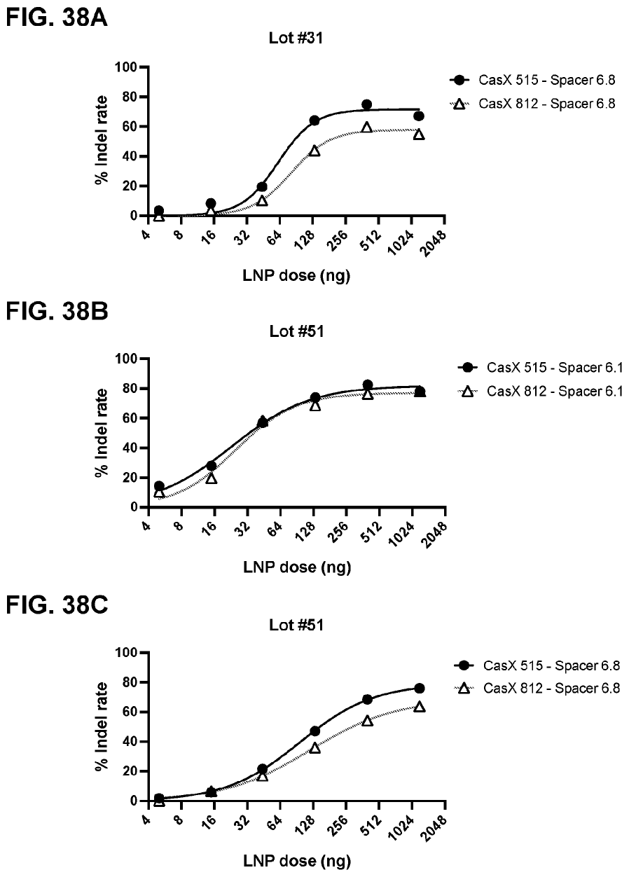

Two lots of primary human hepatocytes were treated with LNPs, which co-encapsulated either CasX 515 or CasX 812 mRNA and a PCSK9-targeting gRNA, at various doses and harvested five days post-treatment to assess effects on PCSK9 secretion (FIGS. 37A-37D) and editing at the PCSK9 locus (FIGS. 38A-38C). The results in FIGS. 37A-37D demonstrate that the effects from treatment with LNPs to deliver either CasX 515 or CasX 812 mRNA were comparable, such that similar levels of reduced PCSK9 secretion were observed in a dose-dependent manner. Furthermore, the data in FIGS. 38A-38C show that use of either CasX 515 or CasX 812 mRNA resulted in similar levels of editing at the PCSK9 locus in primary human hepatocytes in a dose-dependent manner, corroborating findings observed in FIGS. 37A-37D.

Altogether, the results from these experiments demonstrate that delivery of LNPs encapsulating a CasX mRNA and a PCSK9-targeting gRNA was able to induce efficacious editing at the endogenous human PCSK9 locus in primary human hepatocytes, which resulted in substantial reduction in secreted PCSK9 levels.

Example 22: Comprehensive Evaluation of PCSK9-Targeting Spacers with TTC PAMs in Achieving Editing of the Human PCSK9 Locus and Reducing PCSK9 Secretion In Vitro when Paired with CasX 515

Experiments were performed to carry out a comprehensive evaluation of PCSK9-targeting spacers with the TTC recognition motif when paired with CasX 515. Briefly, in vitro experiments were conducted to assess and identify targeting spacers that lead to significant editing of the human PCSK9 locus, substantial reduction in PCSK9 secretion, and minimal level of intracellular protein retention.

### Materials and Methods

Computational Selection of PCSK9-Targeting Spacers for Experimental Testing with CasX 515:

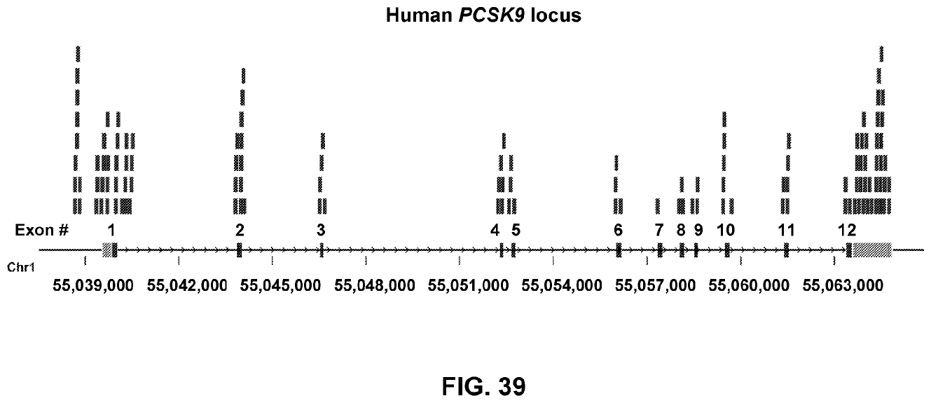

To determine potential spacers throughout the human PCSK9 locus, a target search region was defined as starting at 10 KB upstream of the transcription start site (TSS) through 5 KB downstream of the transcription stop site. Spacers were determined based on the availability of TTC PAMs; consequently, a total of 960 TTC spacers were identified throughout the target PCSK9 locus. These spacers were then functionally annotated by overlaying key genomic features based on their positioning, i.e., determining whether the putative spacer targeted an exon, an intron, or a candidate cis-regulatory element (cCRE), within the promoter region, and/or overlapped with a common site of genetic variation (e.g., SNPs). To narrow down and determine an initial group of spacers for experimental screening, the extracted spacers were subjected to a set of filtering criteria. Firstly, non-specific spacers were excluded by removing spacers with off-target sites that contain up to one base pair mismatch with the on-target site. Furthermore, spacers containing mononucleotide repeats that were greater than four base pairs in length were excluded. Subsequently, from this filtered set, spacers that were functionally annotated to target an exon, the promoter region, a splice acceptor or donor site, or a cCRE of the human PCSK9 locus were selected for inclusion. As a result, a total of 123 TTC spacers were identified, and the sequences are listed in Table 40. FIG. 39 illustrates a schematic of the relative locations in the human PCSK9 gene that these 123 spacers target.

TABLE 40
Sequences of the 123 TTC spacers
targeting the human PCSK9 locus

| Spacer | DNA SEQ | RNA SEQ |
|---|---|---|
| ID | ID NO: | ID NO: |
| 6.87 | 13810 | 544 |
| 6.210 | 13873 | 545 |
| 6.213 | 13876 | 546 |
| 6.214 | 13877 | 547 |
| 6.215 | 13878 | 548 |
| 6.216 | 13879 | 549 |
| 6.10 | 13790 | 550 |
| 6.6 | 13787 | 551 |
| 6.7 | 13788 | 552 |
| 6.114 | 13825 | 553 |
| 6.110 | 13822 | 554 |
| 6.95 | 14109 | 555 |
| 6.113 | 14110 | 556 |
| 6.331 | 14111 | 557 |
| 6.332 | 14112 | 558 |
| 6.154 | 13841 | 559 |
| 6.155 | 13842 | 560 |
| 6.156 | 13843 | 561 |
| 6.157 | 13844 | 562 |
| 6.158 | 13845 | 563 |
| 6.160 | 13846 | 564 |
| 6.161 | 13847 | 565 |
| 6.188 | 13851 | 566 |
| 6.189 | 13852 | 567 |
| 6.190 | 13853 | 568 |
| 6.8 | 13789 | 569 |
| 6.86 | 13809 | 570 |
| 6.191 | 13854 | 571 |
| 6.192 | 13855 | 572 |
| 6.193 | 13856 | 573 |
| 6.194 | 13857 | 574 |
| 6.195 | 13858 | 575 |
| 6.196 | 13859 | 576 |
| 6.197 | 13860 | 577 |
| 6.198 | 13861 | 578 |
| 6.199 | 13862 | 579 |
| 6.200 | 13863 | 580 |
| 6.201 | 13864 | 581 |
| 6.202 | 13865 | 582 |
| 6.203 | 13866 | 583 |
| 6.204 | 13867 | 584 |
| 6.205 | 13868 | 585 |
| 6.206 | 13869 | 586 |
| 6.207 | 13870 | 587 |
| 6.208 | 13871 | 588 |
| 6.209 | 13872 | 589 |
| 6.211 | 13874 | 590 |
| 6.212 | 13875 | 591 |
| 6.217 | 13880 | 592 |
| 6.218 | 13881 | 593 |
| 6.219 | 13882 | 594 |
| 6.220 | 13883 | 595 |
| 6.221 | 13884 | 596 |
| 6.222 | 13885 | 597 |
| 6.223 | 13886 | 598 |
| 6.224 | 13887 | 599 |
| 6.225 | 13888 | 600 |
| 6.226 | 13889 | 601 |
| 6.227 | 13890 | 602 |
| 6.228 | 13891 | 603 |
| 6.229 | 13892 | 604 |
| 6.230 | 13893 | 605 |
| 6.231 | 13894 | 606 |
| 6.232 | 13895 | 607 |
| 6.233 | 13896 | 608 |
| 6.234 | 13897 | 609 |
| 6.235 | 13898 | 610 |
| 6.236 | 13899 | 611 |
| 6.237 | 13900 | 612 |
| 6.238 | 13901 | 613 |
| 6.239 | 13902 | 614 |
| 6.240 | 13903 | 615 |
| 6.241 | 13904 | 616 |
| 6.242 | 13905 | 617 |
| 6.4 | 13784 | 618 |
| 6.1 | 13782 | 619 |
| 6.5 | 13785 | 620 |
| 6.2 | 13783 | 621 |
| 6.118 | 13828 | 622 |
| 6.119 | 13829 | 623 |
| 6.120 | 13830 | 624 |
| 6.122 | 13832 | 625 |
| 6.123 | 13833 | 626 |
| 6.121 | 13831 | 627 |
| 6.124 | 13834 | 628 |
| 6.125 | 13835 | 629 |
| 6.126 | 13836 | 630 |
| 6.127 | 13837 | 631 |
| 6.149 | 13838 | 632 |
| 6.150 | 13839 | 633 |
| 6.151 | 13840 | 634 |
| 6.243 | 13906 | 635 |
| 6.244 | 13907 | 636 |
| 6.245 | 13908 | 637 |
| 6.246 | 13909 | 638 |
| 6.247 | 13910 | 639 |
| 6.248 | 13911 | 640 |
| 6.249 | 13912 | 641 |
| 6.250 | 13913 | 642 |
| 6.251 | 13914 | 643 |
| 6.109 | 13821 | 644 |
| 6.3 | 13786 | 645 |
| 6.9 | 646 | 646 |
| 6.88 | 13811 | 647 |
| 6.93 | 13812 | 648 |
| 6.103 | 13816 | 649 |
| 6.106 | 13818 | 650 |
| 6.107 | 13819 | 651 |
| 6.108 | 13820 | 652 |
| 6.111 | 13823 | 653 |
| 6.112 | 13824 | 654 |
| 6.115 | 13826 | 655 |
| 6.116 | 13827 | 656 |
| 6.333 | 14113 | 657 |
| 6.334 | 14114 | 658 |
| 6.335 | 14115 | 659 |
| 6.336 | 14116 | 660 |
| 6.337 | 14117 | 661 |
| 6.338 | 14118 | 662 |
| 6.339 | 14119 | 663 |
| 6.340 | 14120 | 664 |
| 6.341 | 14121 | 665 |
| 6.159 | 14122 | 2016 |

### Assessment of Editing Activity for PCSK9-Targeting Spacers:

Lentiviral plasmid constructs comprising sequences coding for CasX protein 515, guide scaffold variant 235, and PCSK9-targeting spacers (Table 40) were generated and cloned upstream of a P2A-mScarlet coding region on a lentiviral plasmid using0 standard molecular cloning techniques. Cloned and sequence-validated constructs were subjected to quality assessment prior to transfection into HEK293T cells for lentiviral production following standard methods. Briefly, lentivirus was produced by in a 96-well plate by co-transfecting HEK293T cells with CasX plasmids containing PCSK9-targeting spacers, the lentiviral packaging plasmid, and the VSV-G envelope plasmids using the TransIT®-Lenti transfection reagent. Virus was harvested 48 hours post-transfection and titers were determined approximately by measuring mean mScarlet fluorescence compared to a known standard. Lentiviral particles were concentrated by PEG precipitation.

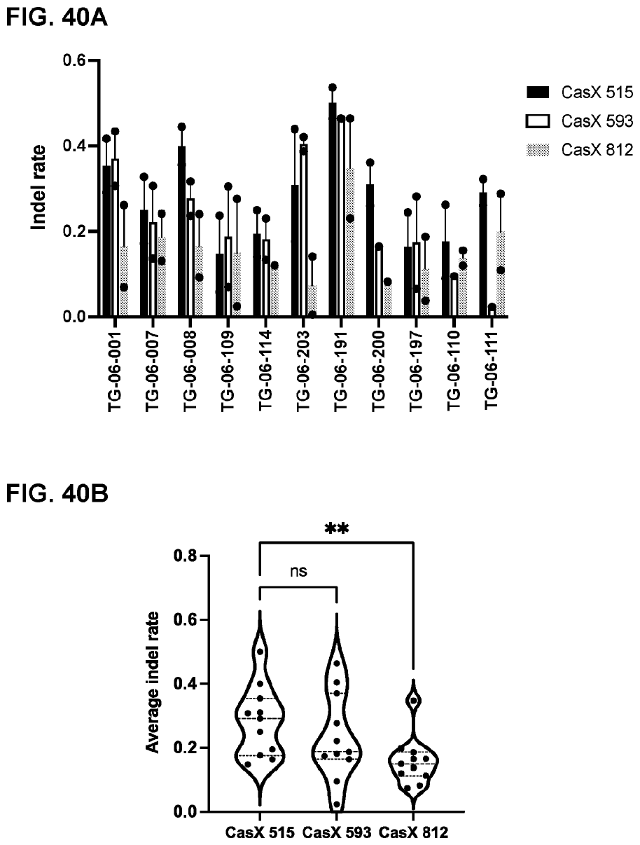

~30,000 HepG2 cells were seeded per well of a 96-well plate; the next day, seeded cells were transduced with lentiviral particles at an MOI of ~1, using DEAE-Dextran to increase transduction efficiency. 72 hours post-transduction, transduced cells were harvested for editing assessment at the PCSK9 locus by NGS following methods as described in Example 4. Three biological replicates of this spacer screen to assess editing activity of candidate targeting spacers were performed. Spacer 6.1, which was shown in previous examples to be an active spacer at the PCSK9 locus, served as an experimental positive control in this experiment. The B2M spacer 7.37 also served as an experimental control in assessing editing activity. Furthermore, a subset of spacers (spacers 6.1, 6.7, 6.8, 6.109, 6.114, 6.203, 6.191, 6.200, 6.197, 6.110, and 6.111) was assessed when paired with CasX 515, 593, and 812 in a separate experiment via lentiviral transduction of HepG2cells. The results of this experiment are shown in Table 41 and FIGS. 40A-40B.

### Assessment of PCSK9 Secretion and Level of Intracellular PCSK9 Protein Retention for Candidate PCSK9-Targeting Spacers:

The level of PCSK9 secretion for the candidate 123 PCSK9-targeting spacers was evaluated by using the CISBio Human PCSK9 HTRF kit following the manufacturer's instructions. Briefly, HepG2 hepatocytes were transduced with lentiviral particles at an MOI of ~5, and 72 hours later, transduction efficiency was evaluated by measuring the level of mScarlet fluorescence via flow cytometry. Wells that measured at least 80% of cells were mScarlet+ were deemed sufficiently transduced, and these cells were cultured for an additional 4-6 days. After a total of 7-10 days post-transduction, the following was harvested: 1) the media supernatant to measure PCSK9 secretion and 2) cell lysates for western blotting (following methods described in Example 20) to evaluate the level of intracellular PCSK9 accumulation. For the ELISA assay, four biological replicates were performed, and for the western blotting, two biological replicates were performed. Spacer 7.37, which targets the human B2M locus, was used as a non-targeting control in assessing PCSK9 secretion and intracellular PCSK9 protein retention levels. For western blotting analysis, two forms of assessments were performed: 1) a qualitative scoring for band intensity by two independent reviewers on a scale of 1 (low PCSK9 protein level) to 5 (high PCSK9 protein level); and 2) quantification of relative band intensity using densitometry relative to the non-targeting control.

Following these phenotypic assays to determine functional effects, a preliminary assessment of off-target editing for a subset of candidate PCSK9-targeting spacers was performed following similar methods as described in Example 10. Three biological experiments of CSI-seq were performed. The data from these experiments were then used to inform the identification and prioritization of leading candidate spacers for targeting the PCSK9 locus for follow-on genotoxicity and in vivo studies.

### Results

123 TTC spacers were tested in a spacer screen to assess their effects on editing activity at the human PCSK9 locus in HepG2 cells transduced with lentiviral particles containing CasX 515 and a PCSK9-targeting gRNA using scaffold variant 235. In this experiment, a 70% lentiviral transduction rate was anticipated. The editing results, which are shown in Table 41 below depicted as indel rate, demonstrate that use of multiple spacers, including spacer 6.1, resulted in a high editing rate (at least 66%, which was observed with use of the B2M 7.37 spacer) when paired with CasX 515.

In the separate experiment testing spacers 6.1, 6.7, 6.8, 6.109, 6.114, 6.203, 6.191, 6.200, 6.197, 6.110, and 6.111 with CasX 515, 593, and 812, the editing results are illustrated in FIGS. 40A-40B. The data show that across multiple spacers, CasX 593 exhibited similar editing activity as CasX 515, while the editing rates for CasX 812 were relatively significantly lower compared to CasX 515 (p<0.01; two-way ANOVA).

TABLE 41
Results of an editing assay evaluating the editing activity
at the PCSK9 locus for the 123 PCSK9-targeting spacers.
Genomic annotations for these spacers are also shown, where
“CDS” denotes coding sequence of the PCSK9 locus

| | Genomic | Mean % | Standard | Sample |
|---|---|---|---|---|
| Spacer ID | feature | editing | error | size |
| TG-06-001 | Exon - CDS | 51.391% | 0.0561 | 3 |
| TG-06-002 | Splice Site | 0.726% | 0.0044 | 3 |
| TG-06-003 | Exon - CDS | 17.968% | 0.0166 | 2 |
| TG-06-004 | Exon - CDS | 19.320% | 0.0923 | 3 |
| TG-06-005 | Exon - CDS | 11.792% | 0.0862 | 3 |
| TG-06-006 | Exon - CDS | 15.855% | 0.0365 | 3 |
| TG-06-007 | Exon - CDS | 35.794% | 0.0112 | 3 |
| TG-06-008 | Exon - CDS | 57.711% | 0.0939 | 3 |
| TG-06-009 | Exon - CDS | 47.746% | N/A | 1 |
| TG-06-010 | Splice Site | 30.872% | 0.0336 | 3 |
| TG-06-086 | Exon - CDS | 67.394% | 0.1712 | 3 |
| TG-06-087 | Splice Site | 40.875% | N/A | 1 |
| TG-06-088 | Intron | N/A | N/A | N/A |
| TG-06-093 | Intron | 17.251% | 0.1104 | 2 |
| TG-06-095 | Exon - CDS | 36.737% | N/A | 1 |
| TG-06-103 | Exon - CDS | 0.289% | N/A | 1 |
| TG-06-106 | Exon - CDS | 0.084% | 0.0003 | 3 |
| TG-06-107 | Exon - CDS | N/A | N/A | N/A |
| TG-06-108 | Exon - CDS | 5.978% | 0.0593 | 2 |
| TG-06-109 | Splice Site | 15.891% | 0.0560 | 3 |
| TG-06-110 | Splice Site | 31.070% | 0.0720 | 3 |
| TG-06-111 | Splice Site | 40.936% | 0.0771 | 3 |
| TG-06-112 | Exon - CDS | N/A | N/A | N/A |
| TG-06-113 | Splice Site | 40.554% | N/A | 1 |
| TG-06-114 | Exon - CDS | 28.007% | 0.0128 | 3 |
| TG-06-115 | Exon - CDS | 12.475% | 0.0020 | 2 |
| TG-06-116 | Exon - CDS | N/A | N/A | N/A |
| TG-06-118 | Promoter | 46.435% | 0.0580 | 3 |
| TG-06-119 | Promoter | 23.351% | 0.0915 | 3 |
| TG-06-120 | Promoter | 45.189% | 0.0708 | 3 |
| TG-06-121 | Promoter | 78.446% | 0.2339 | 3 |
| TG-06-122 | Promoter | 9.503% | 0.0500 | 3 |
| TG-06-123 | Promoter | 33.297% | 0.0065 | 3 |
| TG-06-124 | Promoter | 46.031% | 0.0502 | 3 |
| TG-06-125 | Promoter | 52.699% | 0.0838 | 3 |
| TG-06-126 | Promoter | 6.586% | 0.0422 | 2 |
| TG-06-127 | Promoter | N/A | N/A | N/A |
| TG-06-149 | Promoter | 19.492% | 0.0716 | 3 |
| TG-06-150 | Promoter | 28.915% | 0.0363 | 3 |
| TG-06-151 | Promoter | 36.351% | 0.0110 | 3 |
| TG-06-154 | Promoter | 64.185% | 0.0785 | 2 |
| TG-06-155 | Exon - UTR | 35.668% | 0.0636 | 3 |
| TG-06-156 | Exon - UTR | 47.722% | 0.0938 | 3 |
| TG-06-157 | Exon - UTR | 32.271% | 0.0980 | 3 |
| TG-06-158 | Exon - UTR | 70.636% | 0.1135 | 3 |
| TG-06-159 | Exon - UTR | N/A | N/A | N/A |
| TG-06-160 | Exon - UTR | 28.951% | 0.1814 | 2 |
| TG-06-161 | Exon - UTR | 17.187% | 0.0897 | 3 |
| TG-06-188 | Splice Site | 10.498% | 0.0660 | 3 |
| TG-06-189 | Intron | 9.849% | 0.0135 | 3 |
| TG-06-190 | Intron | 28.489% | 0.0597 | 3 |
| TG-06-191 | Intron | 70.165% | 0.1497 | 3 |
| TG-06-192 | Intron | 16.292% | 0.0167 | 3 |
| TG-06-193 | Intron | 21.971% | 0.0243 | 3 |
| TG-06-194 | Intron | 21.431% | 0.1047 | 3 |
| TG-06-195 | Intron | 24.413% | 0.0297 | 3 |
| TG-06-196 | Intron | 40.615% | 0.0888 | 2 |
| TG-06-197 | Exon - CDS | 15.487% | 0.0784 | 3 |
| TG-06-198 | Splice Site | 20.411% | 0.0355 | 3 |
| TG-06-199 | Exon - CDS | 33.342% | 0.0317 | 3 |
| TG-06-200 | Exon - CDS | 48.161% | 0.0745 | 2 |
| TG-06-201 | Intron | 0.755% | 0.0025 | 3 |
| TG-06-202 | Exon - CDS | 22.386% | 0.0601 | 3 |
| TG-06-203 | Exon - CDS | 55.216% | 0.2159 | 3 |
| TG-06-204 | Intron | 6.913% | 0.0223 | 3 |
| TG-06-205 | Intron | 14.606% | 0.0262 | 3 |
| TG-06-206 | Intron | 24.246% | 0.0521 | 3 |
| TG-06-207 | Splice Site | 4.988% | 0.0149 | 3 |
| TG-06-208 | Splice Site | 13.243% | 0.0412 | 3 |
| TG-06-209 | Intron | 2.935% | 0.0055 | 3 |
| TG-06-210 | Splice Site | 22.824% | 0.0349 | 3 |
| TG-06-211 | Exon - CDS | 32.018% | 0.1195 | 2 |
| TG-06-212 | Exon - CDS | 39.612% | 0.2089 | 2 |
| TG-06-213 | Exon - CDS | 8.929% | 0.0114 | 2 |
| TG-06-214 | Splice Site | 13.003% | 0.0222 | 3 |
| TG-06-215 | Splice Site | 42.823% | 0.1360 | 3 |
| TG-06-216 | Exon - CDS | 18.910% | 0.0737 | 3 |
| TG-06-217 | Exon - UTR | 5.790% | 0.0213 | 3 |
| TG-06-218 | Exon - UTR | 17.055% | 0.0532 | 3 |
| TG-06-219 | Exon - UTR | 36.588% | 0.1278 | 3 |
| TG-06-220 | Exon - UTR | 45.281% | 0.2120 | 2 |
| TG-06-221 | Exon - UTR | 27.551% | 0.0266 | 3 |
| TG-06-222 | Exon - UTR | 3.020% | 0.0035 | 3 |
| TG-06-223 | Exon - UTR | 9.987% | 0.0056 | 3 |
| TG-06-224 | Exon - UTR | 37.368% | 0.0879 | 3 |
| TG-06-225 | Exon - UTR | 12.545% | 0.0086 | 3 |
| TG-06-226 | Exon - UTR | 27.907% | 0.0126 | 3 |
| TG-06-227 | Exon - UTR | 43.516% | 0.0261 | 3 |
| TG-06-228 | Exon - UTR | 20.654% | 0.0244 | 3 |
| TG-06-229 | Exon - UTR | 37.558% | 0.0862 | 3 |
| TG-06-230 | Exon - UTR | 57.782% | 0.1787 | 3 |
| TG-06-231 | Exon - UTR | 38.642% | 0.0286 | 3 |
| TG-06-232 | Exon - UTR | 19.385% | 0.0153 | 3 |
| TG-06-233 | Exon - UTR | 25.004% | 0.0269 | 3 |
| TG-06-234 | Exon - UTR | 53.043% | 0.0810 | 3 |
| TG-06-235 | Exon - UTR | 52.042% | 0.1137 | 3 |
| TG-06-236 | Exon - UTR | 24.875% | N/A | 1 |
| TG-06-237 | Exon - UTR | 22.322% | 0.0037 | 3 |
| TG-06-238 | Exon - UTR | 54.619% | 0.0957 | 3 |
| TG-06-239 | Exon - UTR | 22.849% | 0.0299 | 3 |
| TG-06-240 | Exon - UTR | 44.231% | 0.0970 | 3 |
| TG-06-241 | Exon - UTR | 20.327% | 0.0323 | 3 |
| TG-06-242 | Exon - UTR | 33.495% | 0.0646 | 3 |
| TG-06-243 | Intron | 31.619% | 0.0357 | 3 |
| TG-06-244 | Intron | 23.330% | 0.0728 | 3 |
| TG-06-245 | Intron | 37.414% | 0.0144 | 3 |
| TG-06-246 | Intron | 21.676% | 0.0705 | 3 |
| TG-06-247 | Intron | 19.443% | 0.0990 | 3 |
| TG-06-248 | Intron | 25.689% | 0.0437 | 3 |
| TG-06-249 | Intron | 17.101% | 0.0431 | 3 |
| TG-06-250 | Intron | 4.627% | 0.0221 | 3 |
| TG-06-251 | Intron | 44.929% | 0.0553 | 3 |
| TG-06-331 | Splice Site | 34.165% | N/A | 1 |
| TG-06-332 | Exon - CDS | N/A | N/A | N/A |
| TG-06-333 | Exon - UTR | 30.298% | N/A | 1 |
| TG-06-334 | Exon - UTR | 40.259% | N/A | 1 |
| TG-06-335 | Exon - UTR | 34.313% | N/A | 1 |
| TG-06-336 | Exon - UTR | 42.738% | N/A | 1 |
| TG-06-337 | Exon - UTR | 46.603% | N/A | 1 |
| TG-06-338 | Exon - UTR | 46.123% | N/A | 1 |
| TG-06-339 | Exon - UTR | 40.450% | N/A | 1 |
| TG-06-340 | Exon - UTR | 0.251% | N/A | 1 |
| TG-06-341 | Exon - UTR | 25.547% | N/A | 1 |
| TG-07-037 | Control | 66.384% | 0.1393 | 5 |

*Note that “06-##” nomenclature in Tables 41-44 corresponds to the “6.##” nomenclature in Table 40, which provides the corresponding SEQ ID NOS.

The 123 candidate PCSK9-targeting spacers were evaluated for their effects on PCSK9 secretion using an ELISA assay, and the data, which are depicted as percent reduction of secreted PCSK9 levels compared to the non-targeting control (spacer 7.37), are shown in Table 42. Wells for 113 of the 123 spacers measured at least an 80% transduction efficiency. The results demonstrate that of the 113 spacers evaluated, 44 spacers were initially identified whose use resulted in >50% reduction in secreted PCSK9 protein (Table 42). The 10 spacers that did not meet the threshold of 80% transduction efficiency were subsequently re-evaluated in a separate ELISA experiment. 9 out of the 10 spacers re-evaluated met the 80% transduction efficiency threshold, from which 7 spacers resulted in >50% reduction in secreted PCSK9 protein (Table 42).

TABLE 42
Results of the ELISA assay evaluating the functional effects
of the 123 PCSK9-targeting spacers on PCSK9 secretion levels.
Spacers that demonstrated >50% reduction in PCSK9 secretion
are shown in bold. The 10 spacers that were re-evaluated
in a follow-up experiment are marked with *

| | | | | Mean % |
|---|---|---|---|---|
| | Secreted | Standard | | reduction in |
| | PCSK9 | error | Sample | PCSK9 |
| Spacer ID | (ng/mL) | (ng/mL) | size | secretion |
| TG-06-001 | 9.950 | 0.784 | 33 | N/A |
| TG-06-002 | 13.580 | 2.191 | 3 | −64.672 |
| TG-06-003* | 17.502 | 0.435 | 3 | −54.470 |
| TG-06-004 | 3.264 | 0.560 | 3 | −91.510 |
| TG-06-005 | 4.850 | 1.339 | 3 | −87.383 |
| TG-06-006 | 2.043 | 0.346 | 3 | −94.686 |
| TG-06-007 | 2.252 | 0.556 | 3 | −94.142 |
| TG-06-008 | 1.787 | 0.488 | 3 | −95.352 |
| TG-06-009 | 4.932 | 0.340 | 6 | −87.170 |
| TG-06-010 | 1.772 | 0.314 | 3 | −95.389 |
| TG-06-086 | 2.311 | 1.016 | 3 | −93.988 |
| TG-06-087 | 11.733 | 2.436 | 3 | −69.478 |
| TG-06-088* | N/A | N/A | N/A | N/A |
| TG-06-093 | 4.172 | 0.378 | 2 | −89.147 |
| TG-06-095 | 4.567 | 0.853 | 3 | −88.119 |
| TG-06-103* | 17.266 | 0.827 | 3 | −55.081 |
| TG-06-106* | 12.504 | 0.444 | 4 | −67.469 |
| TG-06-107* | 54.761 | 2.117 | 5 | 42.461 |
| TG-06-108* | 61.417 | 1.509 | 3 | 59.778 |
| TG-06-109 | 31.539 | 2.234 | 6 | −17.949 |
| TG-06-110* | 5.993 | 0.290 | 3 | −84.409 |
| TG-06-111* | 4.692 | 0.554 | 3 | −87.793 |
| TG-06-112* | 11.453 | 0.539 | 3 | −70.205 |
| TG-06-113 | 1.565 | 0.426 | 3 | −95.927 |
| TG-06-114 | 18.456 | 4.429 | 6 | −51.986 |
| TG-06-115 | 19.298 | 1.320 | 5 | −49.797 |
| TG-06-116* | 17.258 | 0.624 | 3 | −55.102 |
| TG-06-118 | 16.985 | 2.129 | 3 | −55.812 |
| TG-06-119 | 29.926 | 4.835 | 3 | −22.148 |
| TG-06-120 | 31.365 | 8.779 | 3 | −18.402 |
| TG-06-121 | 32.112 | 3.620 | 3 | −16.460 |
| TG-06-122 | 34.392 | 5.709 | 3 | −10.529 |
| TG-06-123 | 20.726 | 3.750 | 3 | −46.080 |
| TG-06-124 | 32.663 | 5.898 | 3 | −15.026 |
| TG-06-125 | 38.273 | 9.277 | 3 | −0.433 |
| TG-06-126 | 39.839 | 10.893 | 3 | 3.642 |
| TG-06-127 | 40.805 | 10.345 | 3 | 6.154 |
| TG-06-149 | 29.274 | 5.682 | 3 | −23.844 |
| TG-06-150 | 21.145 | 6.013 | 3 | −44.990 |
| TG-06-151 | 33.433 | 8.547 | 3 | −13.025 |
| TG-06-154 | 24.037 | 3.001 | 3 | −37.468 |
| TG-06-155 | 33.930 | 6.869 | 3 | −11.730 |
| TG-06-156 | 33.993 | 9.003 | 3 | −11.567 |
| TG-06-157 | 36.980 | 8.716 | 3 | −3.796 |
| TG-06-158 | 30.114 | 6.620 | 3 | −21.658 |
| TG-06-159 | 59.608 | 2.041 | 3 | 55.071 |
| TG-06-160 | 47.177 | 9.339 | 3 | 22.731 |
| TG-06-161 | 49.076 | 8.343 | 3 | 27.672 |
| TG-06-188 | 30.262 | 7.315 | 3 | −21.272 |
| TG-06-189 | 24.291 | 7.206 | 3 | −36.808 |
| TG-06-190 | 24.288 | 1.761 | 3 | −36.814 |
| TG-06-191 | 27.784 | 4.974 | 3 | −27.719 |
| TG-06-192 | 31.699 | 7.783 | 3 | −17.534 |
| TG-06-193 | 34.304 | 6.386 | 3 | −10.758 |
| TG-06-194 | 45.547 | 8.814 | 3 | 18.492 |
| TG-06-195 | 5.697 | 1.184 | 3 | −85.179 |
| TG-06-196 | 17.184 | 0.713 | 3 | −55.294 |
| TG-06-197 | 3.948 | 0.824 | 3 | −89.730 |
| TG-06-198 | 3.092 | 0.382 | 3 | −91.957 |
| TG-06-199 | 3.661 | 0.373 | 3 | −90.477 |
| TG-06-200 | 2.592 | 0.397 | 3 | −93.256 |
| TG-06-201 | 46.738 | 11.059 | 3 | 21.589 |
| TG-06-202 | 1.832 | 0.438 | 3 | −95.234 |
| TG-06-203 | 2.336 | 0.284 | 3 | −93.922 |
| TG-06-204 | 23.985 | 1.572 | 3 | −37.603 |
| TG-06-205 | 37.617 | 8.492 | 3 | −2.139 |
| TG-06-206 | 31.803 | 9.594 | 3 | −17.263 |
| TG-06-207 | 21.027 | 4.549 | 3 | −45.299 |
| TG-06-208 | 4.156 | 1.032 | 3 | −89.187 |
| TG-06-209 | 32.131 | 7.371 | 3 | −16.412 |
| TG-06-210 | 3.898 | 0.578 | 3 | −89.859 |
| TG-06-211 | 3.190 | 0.508 | 3 | −91.702 |
| TG-06-212 | 3.860 | 0.583 | 3 | −89.958 |
| TG-06-213 | 3.522 | 0.831 | 3 | −90.837 |
| TG-06-214 | 2.408 | 0.521 | 3 | −93.735 |
| TG-06-215 | 4.054 | 1.316 | 3 | −89.453 |
| TG-06-216 | 2.472 | 0.663 | 3 | −93.568 |
| TG-06-217 | 11.856 | 2.562 | 3 | −69.157 |
| TG-06-218 | 23.885 | 0.551 | 3 | −37.863 |
| TG-06-219 | 29.357 | 3.756 | 3 | −23.627 |
| TG-06-220 | 16.347 | 4.295 | 3 | −57.473 |
| TG-06-221 | 37.395 | 7.498 | 3 | −2.716 |
| TG-06-222 | 28.337 | 4.003 | 3 | −26.282 |
| TG-06-223 | 36.699 | 9.324 | 3 | −4.526 |
| TG-06-224 | 34.746 | 6.657 | 3 | −9.606 |
| TG-06-225 | 44.063 | 12.067 | 3 | 14.630 |
| TG-06-226 | 11.697 | 2.525 | 3 | −69.570 |
| TG-06-227 | 20.306 | 6.714 | 3 | −47.174 |
| TG-06-228 | 25.220 | 3.582 | 3 | −34.389 |
| TG-06-229 | 33.567 | 8.130 | 3 | −12.676 |
| TG-06-230 | 31.081 | 5.739 | 3 | −19.142 |
| TG-06-231 | 35.569 | 6.296 | 3 | −7.467 |
| TG-06-232 | 14.098 | 3.871 | 3 | −63.325 |
| TG-06-233 | 37.950 | 7.514 | 3 | −1.273 |
| TG-06-234 | 34.321 | 10.252 | 3 | −10.712 |
| TG-06-235 | 34.725 | 6.809 | 3 | −9.663 |
| TG-06-236 | 26.113 | 1.772 | 3 | −32.067 |
| TG-06-237 | 16.434 | 5.175 | 3 | −57.247 |
| TG-06-238 | 12.868 | 1.329 | 3 | −66.523 |
| TG-06-239 | 35.818 | 7.348 | 3 | −6.819 |
| TG-06-240 | 35.726 | 3.971 | 3 | −7.058 |
| TG-06-241 | 31.256 | 3.886 | 3 | −18.688 |
| TG-06-242 | 26.251 | 1.918 | 3 | −31.708 |
| TG-06-243 | 46.611 | 11.372 | 3 | 21.258 |
| TG-06-244 | 50.533 | 12.183 | 3 | 31.461 |
| TG-06-245 | 39.968 | 9.024 | 3 | 3.979 |
| TG-06-246 | 48.032 | 4.303 | 6 | 24.957 |
| TG-06-247 | 41.026 | 6.038 | 6 | 6.731 |
| TG-06-248 | 39.183 | 6.365 | 6 | 1.935 |
| TG-06-249 | 46.525 | 6.636 | 6 | 21.036 |
| TG-06-250 | 47.166 | 4.051 | 6 | 22.704 |
| TG-06-251 | 33.527 | 3.345 | 6 | −12.779 |
| TG-06-331 | 4.124 | 0.402 | 3 | −89.270 |
| TG-06-332 | 7.771 | 1.490 | 3 | −79.783 |
| TG-06-333 | 35.152 | 6.949 | 3 | −8.551 |
| TG-06-334 | 9.194 | 4.187 | 3 | −76.082 |
| TG-06-335 | 25.545 | 6.135 | 3 | −33.543 |
| TG-06-336 | 16.551 | 2.900 | 3 | −56.943 |
| TG-06-337 | 16.633 | 5.382 | 3 | −56.730 |
| TG-06-338 | 26.177 | 4.433 | 3 | −31.899 |
| TG-06-339 | 26.251 | 5.751 | 3 | −31.709 |
| TG-06-340 | 41.659 | 5.037 | 6 | 8.376 |
| TG-06-341 | 19.456 | 2.502 | 3 | −49.386 |
| TG-07-037 | 38.439 | 2.202 | 30 | 0.000 |

53 candidate PCSK9-targeting spacers (44 identified in the first ELISA experiment and 9 identified in a follow-up ELISA experiment) were further evaluated by western blotting for intracellular protein retention. Band intensities on the western blots were evaluated using a qualitative scoring method and a quantification method by densitometry, and the results are shown in Table 43. Of the 53 candidate spacers assessed, 26 spacers received a qualitative score of <3, an indication that use of the candidate spacers resulted in the reduction of PCSK9 secretion and low intracellular retention. Further analysis showed that quantitative measurements determined by densitometry corroborated findings from qualitative scoring (Table 43).

TABLE 43
Results of the western blotting assay evaluating the functional
effects of the 53 PCSK9-targeting spacers on intracellular
PCSK9 protein retention. A qualitative score of 1 indicates
low PCSK9 protein level, whereas a score of 5 indicates high
PCSK9 protein level. “Relative band intensity” was
determined by measuring the band intensity for each spacer
condition relative to the non-targeting (spacer 7.37) control

| | WB qualitative | Relative band |
|---|---|---|
| Spacer ID | score | intensity |
| TG-06-001 | 2 | 0.125 |
| TG-06-002 | 3 | 0.332 |
| TG-06-003 | 2 | 0.183 |
| TG-06-004 | 2 | 0.081 |
| TG-06-005 | 2 | 0.184 |
| TG-06-006 | 2 | 0.099 |
| TG-06-007 | 2 | 0.257 |
| TG-06-008 | 5 | N/A |
| TG-06-009 | 5 | 0.680 |
| TG-06-010 | 2 | 0.154 |
| TG-06-086 | 5 | 0.652 |
| TG-06-087 | 1 | 0.040 |
| TG-06-093 | 3 | 0.372 |
| TG-06-095 | 2 | 0.086 |
| TG-06-103 | 3 | 0.110 |
| TG-06-106 | 3 | 0.178 |
| TG-06-107 | 4 | 0.430 |
| TG-06-108 | 4 | 0.158 |
| TG-06-109 | 2 | 0.101 |
| TG-06-110 | 1 | 0.051 |
| TG-06-111 | 2 | 0.075 |
| TG-06-112 | 2 | 0.186 |
| TG-06-113 | 1 | 0.039 |
| TG-06-114 | 1 | 0.133 |
| TG-06-115 | 3 | 0.244 |
| TG-06-116 | 3 | 0.225 |
| TG-06-195 | 2 | 0.053 |
| TG-06-196 | 4 | 0.143 |
| TG-06-197 | 5 | 0.491 |
| TG-06-198 | 5 | 0.948 |
| TG-06-199 | 4 | 0.506 |
| TG-06-200 | 4 | 0.239 |
| TG-06-202 | 4 | 0.477 |
| TG-06-203 | 3 | 0.134 |
| TG-06-208 | 3 | 0.183 |
| TG-06-210 | 1 | 0.029 |
| TG-06-211 | 4 | 0.374 |
| TG-06-212 | 4 | 0.396 |
| TG-06-213 | 2 | 0.047 |
| TG-06-214 | 2 | 0.052 |
| TG-06-215 | 2 | 0.057 |
| TG-06-216 | 1 | 0.025 |
| TG-06-217 | 2 | 0.073 |
| TG-06-220 | 4 | 0.260 |
| TG-06-226 | 2 | 0.097 |
| TG-06-232 | 2 | 0.156 |
| TG-06-238 | 3 | 0.195 |
| TG-06-331 | 2 | 0.095 |
| TG-06-332 | 3 | 0.147 |
| TG-06-334 | 1 | 0.082 |
| TG-06-336 | 3 | 0.120 |
| TG-06-340 | 4 | 0.200 |
| TG-06-341 | 3 | 0.354 |

The 26 spacers that demonstrated reduced PCSK9 secretion and low intracellular retention from the western blotting analyses were subsequently evaluated in a preliminary CSI-seq experiment to assess their potential off-targeting effects. Furthermore, 8 additional spacers were included for assessment in a preliminary CSI-seq experiment for comparison. The CSI-seq results, which are presented as the percentage of total off-target reads divided by the total of on-target reads for that targeting spacer, are presented in Table 44. The preliminary CSI-seq data show that multiple spacers appeared to have a relatively comparable or reduced off-target profile when compared to that exhibited by spacer 6.1, a previously identified spacer that demonstrated high activity at the PCSK9 locus. For spacer 6.217, the calculated percentage of total off-target reads over total on-target reads was not available given poor sequencing quality.

After evaluating all the data obtained from assessing editing activity (Table 41), reduction on PCSK9 secretion (Table 42), intracellular PCSK9 retention (Table 43), and preliminary off-target profiles (Table 44), a total of 18 spacers (SEQ ID NO: 544-559; 583; 627) were selected to be subjected to follow-on genotoxicity and in vivo studies.

TABLE 44
Results of the CSI-seq assay evaluating the off-
targeting effects of the 34 PCSK9-targeting spacers

| | % total off-target |
|---|---|
| | reads over total |
| Spacer ID | on-target reads |
| TG-06-210 | 0.11 |
| TG-06-004 | 0.2 |
| TG-06-111 | 0.22 |
| TG-06-112 | 0.23 |
| TG-06-010 | 0.3 |
| TG-06-005 | 0.49 |
| TG-06-214 | 1.2 |
| TG-06-109 | 1.53 |
| TG-06-113 | 1.97 |
| TG-06-006 | 4.3 |
| TG-06-332 | 4.4 |
| TG-06-115 | 6.15 |
| TG-06-216 | 6.21 |
| TG-06-008 | 6.35 |
| TG-06-087 | 7.66 |
| TG-06-110 | 8.79 |
| TG-06-213 | 9.5 |
| TG-06-095 | 11.65 |
| TG-06-114 | 15.69 |
| TG-06-215 | 20.13 |
| TG-06-191 | 25.95 |
| TG-06-007 | 31.99 |
| TG-06-200 | 35.75 |
| TG-06-203 | 36.99 |
| TG-06-093 | 43.6 |
| TG-06-001 | 48.14 |
| TG-06-226 | 53.11 |
| TG-06-003 | 67.02 |
| TG-06-088 | 75.3 |
| TG-06-195 | 159.8 |
| TG-06-331 | 252.04 |
| TG-06-334 | 1912.08 |
| TG-06-232 | 2600.68 |
| TG-06-217 | N/A |

The results from these experiments show that CasX 515 was able to edit the PCSK9 locus with gRNAs with a variety of spacer sequences. Leading candidate PCSK9-targeting spacers were identified, based on their ability to induce effective editing of the human PCSK9 locus, substantial reduction in PCSK9 secretion, and exhibit low intracellular protein retention. The data from a preliminary experiment also revealed spacers with a relatively off-target profile.

Example 23: Use and Functional Assessment of CasX:gRNA Systems Targeting the Human PCSK9 Locus

Experiments are performed to test the ability of CasX:gRNA systems to edit the human PCSK9 locus to reduce PCSK9 secretion. CasX variant 515 and guide scaffold variant 316 are used in these experiments, along with the top 18 PCSK9-targeting spacer candidates identified in Example 22.

### Materials and Methods

Briefly, primary human hepatocytes are treated with varying concentrations of LNPs, which are prepared in ten 3-fold serial dilutions starting at 1324 ng. These LNPs are formulated using Gen Voy-ILM™ lipids following methods described in Example 12 to encapsulate CasX 515 mRNA and a PCSK9-targeting gRNA incorporating scaffold variant 316 with a v1 modification (discussed in Example 6). The sequence for CasX 515 mRNA is shown in Tables 21A-21B. Media is changed 24 hours after LNP treatment, and cells are cultured for an additional 2 days prior to harvesting for gDNA extraction for editing assessment at the PCSK9 locus by NGS following methods described in Example 4. Concurrently with the cell harvesting, the media supernatant is also harvested to measure PCSK9 secretion levels using the LEGEND MAX™ Human PCSK9 ELISA kit following the manufacturer's instructions. Treated cells are also harvested for western blotting analysis to evaluate intracellular PCSK9 cells following methods described in Example 20.

In addition to the functional assays described above to evaluate use of CasX 515 mRNA and identify the top 4-5 leading PCSK9-targeting spacers, an LDL uptake assay is performed as an orthogonal assay to assess that use of the CasX:gRNA system also results in increase in LDLR activity. Briefly, hepatocytes are treated with a single high concentration of LNPs. These LNPs are formulated using GenVoy-ILM™ lipids following methods described in Example 12 to encapsulate CasX 515 mRNA and a PCSK9-targeting gRNA incorporating scaffold variant 316 with a v1 modification (discussed in Example 6). Media is changed 24 hours after LNP treatment, and cells are cultured for an additional 2 days. Cells are then serum starved for 18 hours to clear remaining native LDL from culture conditions. Cell media is replaced with serum-free media containing LDL fluorescently labeled with BODIPY™ (Invitrogen™, No. L3483). After a 3 hr incubation, the cells will be stained for cell surface LDLR. After staining, cells are assessed through live-cell flow cytometry, where LDLR fluorescence intensity and internalized LDL fluorescence intensity is used as a quantitative readout to compare levels of LDLR and LDL uptake in the PCSK9-targeting spacers and its controls.

The results of the assays described herein are used to evaluate the use of CasX variant 515 and to identify PCSK9-targeting spacers that are highly effective for editing the human PCSK9 locus and achieving a therapeutically relevant reduction of PCSK9 secretion that would increase LDLR levels and activity. These leading targeting spacers would subsequently be tested in preclinical in vivo efficacy studies to identify the leading PCSK9-targeting spacer for potential therapeutic use.

Example 24: CasX:gRNA In Vitro Cleavage Assays

Experiments were performed to assess in vitro DNA cleavage by CasX:gRNA ribonucleoproteins (RNPs).

### Materials and Methods

### Assembly of RNP:

RNPs of either CasX 119 (SEQ ID NO: 8), CasX 491 (SEQ ID NO: 4), CasX 515 (SEQ ID NO: 5), or CasX 812 (SEQ ID NO: 7) were assembled with single guide RNAs (sgRNA) with scaffold 316 (SEQ ID NO: 466) and one of two spacers, as described in detail below. The amino acid sequences of CasX 119 and CasX 491 are disclosed in International Publication No. WO2020247882A1. Separately, RNPs of CasX 515 were assembled with sgRNA with either scaffold 2 (SEQ ID NO: 452), 174 (SEQ ID NO: 464), 235 (SEQ ID NO: 465), or 316 and one of two spacers.

Purified RNP of CasX and sgRNA were prepared same-day prior to experiments. For experiments where protein variants were being compared, the CasX protein was incubated with sgRNA at 1:1.2 molar ratio. When scaffolds were compared, the protein was added in 1.2:1 ratio to guide. Briefly, sgRNA was added to Buffer #1 (20 mM Tris HCl pH 7.5, 150 mM NaCl, 1 mM TCEP, 5% glycerol, 10 mM MgCl₂) on ice, then CasX was added to the sgRNA solution, slowly with swirling, and immediately incubated at 37° C. for 20 minutes to form RNP complexes. RNP complexes were centrifuged at 4° C. for 5 minutes at 16,000×g to remove any precipitate. Formation of competent (active) RNP was assessed as described below.

### In Vitro Cleavage Assays:

The ability of CasX variants to form active RNP compared to reference CasX was determined using an in vitro cleavage assay. The beta-2 microglobulin (B2M) 7.9 and 7.37 target for the cleavage assay was created as follows. DNA oligos (sequences in Table 45) were generated with 5′ terminal amino modification for conjugation to Cy-dyes with an amino-reactive handle (N-hydroxysuccinimide). Oligo-dye conjugation reactions of 100 μM oligo and 1 mM dye were performed in 100 mM sodium borate pH 8.3 at 4° C. for 16 h. Target strands (TS) were labeled with Cy5.5 and non-targeting strands (NTS) were labeled with Cy7.5. After quenching the reactions with 1 mM Tris pH 7.5, the conjugated oligos were purified via ethanol precipitation. Double-stranded DNA (dsDNA) targets were formed by mixing the oligos in a 1:1 ratio in 1× hybridization buffer (20 mM Tris HCl pH 7.5, 100 mM KCl, 5 mM MgCl₂), heating to 95° C. for 10 minutes, and allowing the solution to cool to room temperature.

TABLE 45
DNA sequences and descriptions of target DNAs

| DNA Sequence (5′-3′)* | SEQ ID NO: | Description |
|---|---|---|
| /5AmMC6/TGAAGCTGACAGCATTCGGGCCGAGA | 14336 | 7.37 NTS with 5′ amine for NHS ester |
| TGTCTCGCTCCGTGGCCTTAGCTGTGCTCGCGCT | | linkages ** |
| /5AmMC6/AGCGCGAGCACAGCTAAGGCCACGGA | 14337 | 7.37 TS with 5′ amine |
| GCGAGACATCTCGGCCCGAATGCTGTCAGCTTCA | | |
| /5AmMC6/TGAAGCTGACAGCATTCGGGCCTAGA | 14338 | 7.37 gel probe NTS, mismatch at 5 |
| TGTCTCGCTCCGTGGCCTTAGCTGTGCTCGCGCT | | |
| /5AmMC6/AGCGCGAGCACAGCTAAGGCCACGGA | 14339 | 7.37 gel probe TS, mismatch at 5 |
| GCGAGACATCTAGGCCCGAATGCTGTCAGCTTCA | | |
| /5AmMC6/TGAAGCTGACAGCATTCGGGCCGAGA | 14340 | 7.37 gel probe NTS, mismatch at 10 |
| TATCTCGCTCCGTGGCCTTAGCTGTGCTCGCGCT | | |
| /5AmMC6/AGCGCGAGCACAGCTAAGGCCACGGA | 14341 | 7.37 gel probe TS, mismatch at 10 |
| GCGAGATATCTCGGCCCGAATGCTGTCAGCTTCA | | |
| /5AmMC6/TGAAGCTGACAGCATTCGGGCCGAGA | 14342 | 7.37 gel probe NTS, mismatch at 15 |
| TGTCTCGATCCGTGGCCTTAGCTGTGCTCGCGCT | | |
| /5AmMC6/AGCGCGAGCACAGCTAAGGCCACGGA | 14343 | 7.37 gel probe TS, mismatch at 15 |
| TCGAGACATCTCGGCCCGAATGCTGTCAGCTTCA | | |
| /5AmMC6/CTTTCAGCAAGGACTGGTCTTTCTAT | 14344 | 7.9 target TS |
| CTCTTGTACTACACTGAATTCACCCCCACTGAAA | | |
| /5AmMC6/TTTCAGTGGGGGTGAATTCAGTGTAG | 14345 | 7.9 target NTS |
| TACAAGAGATAGAAAGACCAGTCCTTGCTGAAAG | | |
| /5AmMC6/CTTTCAGCAAGGACTGGTCTTTCTAT | 14346 | 7.9 gel probe TS, mismatch at 5 |
| CTCTTGTACGACACTGAATTCACCCCCACTGAAA | | |
| /5AmMC6/TTTCAGTGGGGGTGAATTCAGTGTCG | 14347 | 7.9 gel probe NTS, mismatch at 5 |
| TACAAGAGATAGAAAGACCAGTCCTTGCTGAAAG | | |
| /5AmMC6/CTTTCAGCAAGGACTGGTCTTTCTAT | 14348 | 7.9 gel probe TS, mismatch at 10 |
| CTCTCGTACTACACTGAATTCACCCCCACTGAAA | | |
| /5AmMC6/TTTCAGTGGGGGTGAATTCAGTGTAG | 14349 | 7.9 gel probe NTS, mismatch at 10 |
| TACGAGAGATAGAAAGACCAGTCCTTGCTGAAAG | | |
| /5AmMC6/CTTTCAGCAAGGACTGGTCTTTCTGT | 14350 | 7.9 gel probe TS, mismatch at 15 |
| CTCTTGTACTACACTGAATTCACCCCCACTGAAA | | |
| /5AmMC6/TTTCAGTGGGGGTGAATTCAGTGTAG | 14351 | 7.9 gel probe NTS, mismatch at 15 |
| TACAAGAGACAGAAAGACCAGTCCTTGCTGAAAG | | |

*5AmMC6 indicates the 5′ Amino Modifier C6. The target sequences are underlined.
** The Kcleave assay using the mismatched position 5 dsDNA target was run at 37° C.

### Determining Cleavage-Competent Fractions for RNPs:

Cleavage reactions were prepared with final RNP concentrations of 100 nM and final target concentration of 100 nM. Reactions were carried out at 37° C. and initiated by the addition of the dye-labeled dsDNA target. Aliquots were taken at 5, 30, and 60 minutes and quenched by adding to 95% formamide, 25 mM EDTA. Samples were denatured by heating at 95° C. for 10 minutes and run on a 10% urea-PAGE gel. The gels were imaged with a Cytiva Typhoon and quantified using the Cytiva IQTL software.

### Kcleave Assay:

Cleavage reactions were set up with a final RNP concentration of 200 nM and a final target concentration of 10 nM. Reactions were carried out at 16° C., except where otherwise noted, and initiated by the addition of the target DNA. Aliquots were taken at 15, 30, 60, 120, 180, 240, and 480 seconds, and quenched by adding to 95% formamide, 25 mM EDTA. Samples were denatured by heating at 95° C. for 10 minutes and run on a 10% urea-PAGE gel. The gels were imaged with a Cytiva Typhoon and quantified using the Cytiva IQTL software. The apparent first-order rate constant of non-target strand cleavage (k_{cleave}) was determined for each CasX:sgRNA combination replicate individually.

To test the relative specificities of engineered proteins in vitro, apparent cleavage rate constants were compared for targets with mismatched bases at various positions (5, 10, and 15 nt downstream of PAM, Table 45). Cleavage assays were performed in large excess of RNP (200 nM RNP and 1 nM target dsDNA) at 16° C., with the exception of assays measuring cleavage of the target with a mismatch at 5 nt, which were conducted at 37° C. in order to observe measurable cleavage rates. Aliquots were taken at 15, 30, 60, 120, 180, 240, and 480 seconds, and quenched by adding to 95% formamide, 25 mM EDTA. Samples were denatured by heating at 95° C. for 10 minutes and run on a 10% urea-PAGE gel. The gels were imaged with a Cytiva Typhoon and quantified using the Cytiva IQTL software. The apparent first-order rate constant of non-target strand cleavage (k_{cleave}) was determined for each CasX:sgRNA combination replicate individually.

### Results

### Determining Cleavage-Competent Fractions for Protein Variants Compared to Reference CasX 119:

To determine the cleavage-competent fraction for the tested CasX proteins, it was assumed that CasX acts essentially as a single-turnover enzyme under the assayed conditions, as indicated by the observation that sub-stoichiometric amounts of enzyme fail to cleave a greater-than-stoichiometric amount of target even under extended time-scales and instead approach a plateau that scales with the amount of enzyme present. Thus, the fraction of target cleaved over long time-scales by an equimolar amount of RNP is indicative of what fraction of the RNP is properly formed and active for cleavage. Thus, the active (competent) fraction for each RNP was derived from the cleaved fraction over the total signal at the 60-minute timepoint, upon confirming an increase in cleaved fraction from the 5-minute timepoint, and relative plateau in cleaved fraction from the 30-minute timepoint.

Apparent competent fractions were determined for the RNPs with various CasX proteins, and are provided in Table 46.

TABLE 46
Protein variant RNP comparison of fraction
competence and Kcleave rates

| | | Kcleave (sec⁻¹) | | | |
|---|---|---|---|---|---|
| | Fraction | | Position | Position | Position |
| RNP | competence* | On- | 15 mis- | 10 mis- | 5 mis- |
| (CasX.Scaf- | (mean ± | target | match | match | match |
| fold.Spacer) | stdev) | spacer | spacer | spacer | spacer** |
| 119.174.7.9 | 26.71 ± 9.65% | 0.0292 | 0.0158 | 0.0047 | 0.0127 |
| 491.174.7.9 | 49.23 ± 6.37% | 0.0966 | 0.0552 | 0.03 | 0.0616 |
| 515.174.7.9 | 30.15 ± 3.58% | 0.0782 | 0.0625 | 0.0324 | 0.0392 |
| 812.174.7.9 | 53.28 ± 5.14% | 0.0909 | 0.0512 | 0.0142 | 0.01 |
| 119.174.7.37 | 29.01 ± 2.67% | 0.0022 | 0.0061 | 0.0028 | 0.0034 |
| 491.174.7.37 | 48.73 ± 5.44% | 0.0425 | 0.015 | 0.0297 | 0.0301 |
| 515.174.7.37 | 43.54 ± 16.19% | 0.0396 | 0.0127 | 0.0297 | 0.0347 |
| 812.174.7.37 | 38.23 ± 2.49% | 0.027 | 0.0111 | 0.0119 | 0.014 |

*Active fraction was calculated by averaging three experimental replicates.
**The Kcleave assay using the mismatched position 5 dsDNA target was run at 37° C.

For protein variant comparison, the following CasX proteins were used with guide scaffold 316 and spacer 7.9 or guide 316 and spacer 7.37: CasX 119, CasX 491, CasX 515, and CasX 812. CasX 119 had the lowest active fraction for both spacers, indicating that CasX 491, CasX 515, and CasX 812 form more active and stable RNP with the identical guides under the tested conditions as compared to CasX 119. CasX proteins 491, 515, and 812 did not show consistent trends in their competent fractions across the two spacers, consistent with the expectation that the additional engineering following CasX 491 primarily affects target engagement and cleavage, rather than guide binding or stability.

Kcleave Assay to Understand Specificity of RNPs Formed from Protein Variants:

Assays were performed to measure the apparent first-order rate constant of non-target strand cleavage (k_{cleave}), and the results are presented in Table 46, above. A drastic effect on the kinetics of CasX 812 RNP cleavage was observed for on-target versus the mismatched dsDNA target for both spacers. CasX 812 had comparable on-target cleavage rates to CasX 491 and CasX 515 for both spacers, with a slightly higher cleavage rate than 515 on spacer 7.9, which might be explained by the lower competent fraction observed for the 515 RNP with that spacer, and a lower cleavage rate on 7.37.

The off-target rates for CasX 812 were much more substantially reduced for most of the mismatched substrates. The difference in k_{cleave} rates was readily apparent for the target with a mismatch at position 10, with 812 having a roughly 6-fold (7.9) and 2-fold (7.37) reduction in cleavage rate, as compared to its on-target rate. CasX 515, by comparison, exhibited a 2.4-fold and a 25% reduction on the same targets. A substantial difference was also observed for the position 5 mismatch targets. Even though the assay was run at 37° C. to enable measurable cleavage rates, as the position 5 mismatch targets were essentially uncleaved by the CasX RNPs at the lower temperature used for the other targets, CasX 812 against spacer 7.9 exhibited a 9-fold reduction in cleavage rate from on-target rate run at 16° C. and a 2-fold reduction for the 7.37 spacer with a position 5 mismatch. CasX 515 showed a 2-fold reduction for mismatched 7.9 and a nearly equivalent cleavage rate for 7.37 with the position 5 mismatch (note that the “equivalent” cleavage rate is due to the increased temperature).

For the position 15 mismatch substrate, CasX 812 exhibited modest reductions in cleavage rates relative to on-target rates, comparable to the reduction observed for 515. This suggests that the increased sensitivity of CasX 812 to mismatches declines by the PAM distal region, at least for the specific mismatches and spacers tested here. The increased sensitivity at positions 5 and 10 in particular correlates with the position of the G329K mutation present in CasX 812. This mutation introduces a positive charge near the RNA spacer around position 8 and may help CasX to better read out distortions caused by mismatches. Mismatches closer to this new site of contact would be more likely to significantly disrupt either R-loop propagation or allosteric activation of the RuvC (depending on the precise mechanism of increased specificity), while mismatches farther away (as in the position 15 mismatch) might have more variable effects depending on the nature of the mismatch and its effects on the broader heteroduplex structure. Taken together, these data confirm that CasX 812 is inherently more sensitive to mismatches between the RNA spacer and the DNA target and is not simply a less active enzyme, as the decrease in cleavage rate at mismatched targets is in excess of the decrease in cleavage rate at properly matched targets. This is consistent with the results described in Examples 25 and 26 that indicate that CasX 812 is a highly specific enzyme, with lower off-target editing compared to the other nucleases tested.

### Determining Cleavage-Competent Fractions for Single Guide Variants Relative to Reference Single Guide 2:

RNPs were complexed using the aforementioned methods. To isolate the effect of sgRNA identity on RNP formation, guide-limiting conditions were employed. sgRNAs with scaffolds 2, 174, 235, or 316 with spacers 7.9 or 7.37 were mixed with CasX 515 at final concentrations of 1 μM for the guide and 1.2 μM for the protein. Fraction competence was calculated as described above, and the results are provided in Table 47.

TABLE 47
Guide variant RNP comparison of fraction
competence and Kcleave assay

| | Competent | |
|---|---|---|
| RNP construct | fraction | |
| (CasX.Scaf- | (mean ± | Kcleave |
| fold.Spacer) | stdev) | (sec⁻¹) |
| 515.2.7.9 | 28.146.87% | 0.1346 ± 0.0118 |
| 515.174.7.9 | 42.779.62% | 0.1723 ± 0.0046 |
| 515.235.7.9 | 34.111.15% | 0.1696 ± 0.0571 |
| 515.316.7.9 | 30.896.87% | 0.1413 ± 0.0301 |
| 515.2.7.37 | 10.422.24% | 0.0204 ± 0.0002 |
| 515.174.7.37 | 19.962.88% | 0.0534 ± 0.0200 |
| 515.235.7.37 | 32.6411.60% | 0.0647 ± 0.0163 |
| 515.316.7.37 | 26.9811.08% | 0.0851 ± 0.0071 |

* active fraction was calculated by averaging two experimental replicates

Given the complex folding structure of the CasX guide, fraction competence is expected to largely be determined by how much of the guide is properly folded for interaction with the protein. All guides with engineered scaffolds showed improvements over scaffold 2, but guides with scaffold 235 or 316 showed improvements relative to 174 for spacer 7.37. This is consistent with the introduction of mutations in the pseudoknot and triplex that are expected to stabilize the properly folded form.

Higher competent fractions of all guides were observed for spacer 7.9. For this spacer, scaffold 174 had the highest competent fraction, followed by scaffolds 316, 235, and 2. Proper guide folding is expected to be highly dependent on the potential for undesired interactions between the scaffold and spacer sequences, so the observed differences may be attributable to differential sequence-specific interactions, variations in prep quality, or noise in the assay.

Determining k_{cleave} for Single Guide Variants Compared to Reference Scaffold 2:

Cleavage assays were performed with CasX 515 and guides with reference scaffold 2 compared to guides with scaffolds 174, 235, or 316 with spacer 7.9 or 7.37 to determine relative cleavage rates. The mean and standard deviation of three replicates with independent fits are presented in Table 47, above.

To reduce cleavage kinetics to a range measurable with the assay, the cleavage reactions were incubated at 16° C. Under these conditions, all guides supported faster cleavage rates as compared to scaffold 2. For spacer 7.37, the cleavage kinetics aligned with those guides that contributed to the highest fraction competence, with the highest cleavage rate being sg174 (0.1723 s⁻¹), followed by scaffold 235 (0.1696 s⁻¹) and scaffold 316 (0.1413 s⁻¹), versus scaffold 2 (0.1346 s⁻¹). For spacer 7.9, scaffold 316 yielded the highest cleavage rate (0.0851 s⁻¹), followed by scaffold 235 (0.0647 s⁻¹) and sg174 (0.0534 s⁻¹), versus scaffold 2 (0.0204 s⁻¹). The fraction competence and k_{cleave} data did not demonstrate differences across the engineered variants that were consistent across both spacers, although all are consistently better than scaffold 2. This suggests that the improvements seen for scaffold 235 and 316 over 174 are primarily due to behavior in the cell, whether it be stability in the cytoplasm, folding in the cytoplasm, transcription when delivered via plasmid or AAV, or refolding ability when delivered via LNP, that are not captured by guides that have been in vitro transcribed, refolded, and tested for cleavage biochemically.

Example 25: Identification of CasX Proteins with Enhanced Activity or Specificity Relative to CasX 515

An experiment was performed to identify CasX proteins with single mutations and increased editing activity or improved specificity relative to CasX 515.

### Materials and Methods

A multiplexed pooled approach was taken to assay clonal proteins derived from CasX 515 using a pooled activity and specificity (PASS) assay. A pooled HEK cell line, which was adapted to suspension culture from adherent cells, was generated and termed PASS_V1.03. Methods to complete the production of the PASS_V1.03 line were previously described in International Publication No. WO2022120095A1, incorporated herein by reference.

CasX proteins were expressed using a relatively weakly-expressing promoter to reduce CasX protein expression and thereby improve the sensitivity of the assay. Samples were tested in quadruplicate. The list of CasX proteins tested and their mutations relative to CasX 515 is provided in Tables 48 and 49, below. All of the tested CasX proteins had single mutations (i.e., a single amino acid substitution, deletion, or insertion) relative to CasX 515, except for CasX 676, which has three mutations relative to CasX 515. Streptococcus pyogenes Cas9 without a guide RNA served as a negative control.

To assess the editing activity and specificity of the tested CasX proteins at human target sites, two sets of target sites were quantified. First, editing was quantified at TTC PAM on-target sites in which the twenty nucleotides of each gRNA spacer targeting these on-target sites were perfectly complementary to the target site. For each sample and spacer-target pair, data based on<500 reads were removed. Fraction indel values for each sample and spacer-target pair were subtracted by the average fraction indel value across Cas9-treated samples with the same spacer-target pair; Cas9 served as a negative control due to the absence of a compatible guide RNA. Second, editing was quantified at TTC PAM off-target sites, in which one of the twenty nucleotides of the spacer was mismatched with the target site. As above, for each sample and spacer-target pair, data based on<500 reads were removed, and fraction indel values for each sample and spacer-target pair were subtracted by the average fraction indel value across Cas9-treated samples with the same spacer-target pair. Finally, for those TTC PAM spacer-target pairs that had both an on-target and an off-target version, the average editing activity and standard error of the mean (SEM) were calculated.

### Results

Table 48 provides the level of on-target editing produced by various CasX proteins with mutations relative to CasX 515, ranked from highest to lowest activity.

TABLE 48
Average on-targeting editing activity,
ranked from highest to lowest

| Protein | Mutation relative | Average on-target | SEM on-target |
|---|---|---|---|
| Designation | to CasX 515* | TTC PAM editing | TTC PAM editing |
| (CasX protein | (Position.Refer- | activity | activity |
| no., or Cas9) | ence.Alternative) | (fraction) | (fraction) |
| 607 | 398.Y.T | 2.72E−01 | 4.08E−02 |
| 532 | 27.—.R | 2.59E−01 | 3.34E−02 |
| 676 | 27.—.R + | 2.36E−01 | 3.42E−02 |
| | 170.L.K + | | |
| | 224.G.S | | |
| 592 | 304.M.T | 2.19E−01 | 3.73E−02 |
| 788 | 891.S.Q | 2.18E−01 | 3.43E−02 |
| 583 | 169.L.K | 2.17E−01 | 3.66E−02 |
| 555 | 171.A.D | 2.14E−01 | 3.73E−02 |
| 515 | — | 2.09E−01 | 3.82E−02 |
| 569 | 9.K.G | 2.08E−01 | 3.12E−02 |
| 787 | 826.V.M | 2.07E−01 | 3.45E−02 |
| 561 | 5.—.G | 1.98E−01 | 3.41E−02 |
| 577 | 64.R.Q | 1.94E−01 | 3.48E−02 |
| 585 | 171.A.S | 1.94E−01 | 3.40E−02 |
| 572 | 35.R.P | 1.89E−01 | 3.95E−02 |
| 536 | 224.G.T | 1.88E−01 | 3.43E−02 |
| 656 | 887.T.D | 1.87E−01 | 3.34E−02 |
| 559 | 4.I.G | 1.87E−01 | 3.22E−02 |
| 777 | 169.L.Q | 1.84E−01 | 3.36E−02 |
| 584 | 171.A.Y | 1.83E−01 | 3.66E−02 |
| 779 | 372.G.I | 1.79E−01 | 3.15E−02 |
| 566 | 7.I.A | 1.77E−01 | 3.37E−02 |
| 638 | 481.E.D | 1.77E−01 | 3.37E−02 |
| 593 | 304.M.W | 1.76E−01 | 3.35E−02 |
| 568 | 8.N.S | 1.76E−01 | 3.34E−02 |
| 562 | 5.K.G | 1.74E−01 | 3.04E−02 |
| 564 | 6.—.G | 1.73E−01 | 3.32E−02 |
| 757 | 796.K.Q | 1.73E−01 | 2.94E−02 |
| 654 | 772.M.S | 1.70E−01 | 3.18E−02 |
| 760 | 797.T.V | 1.70E−01 | 3.12E−02 |
| 818 | 698.S.R | 1.67E−01 | 3.31E−02 |
| 646 | 653.—.T | 1.66E−01 | 3.06E−02 |
| 784 | 570.P.I | 1.65E−01 | 2.64E−02 |
| 762 | 793.—.P | 1.64E−01 | 2.99E−02 |
| 789 | 917.G.E | 1.61E−01 | 2.91E−02 |
| 649 | 655.—.S | 1.58E−01 | 3.13E−02 |
| 594 | 329.G.N | 1.56E−01 | 3.26E−02 |
| 604 | 342.—.A | 1.55E−01 | 2.87E−02 |
| 612 | 412.G.P | 1.55E−01 | 2.81E−02 |
| 644 | 593.Q.V | 1.52E−01 | 2.97E−02 |
| 736 | 794.P.A | 1.52E−01 | 2.85E−02 |
| 790 | 951.—.S | 1.51E−01 | 2.87E−02 |
| 780 | 389.—.V | 1.49E−01 | 3.02E−02 |
| 717 | 390.K.Q | 1.43E−01 | 2.84E−02 |
| 633 | 473.—.D | 1.41E−01 | 2.71E−02 |
| 590 | 289.K.S | 1.40E−01 | 2.92E−02 |
| 657 | 893.—.N | 1.39E−01 | 2.96E−02 |
| 643 | 593.Q.F | 1.38E−01 | 2.80E−02 |
| 544 | 232.D.G | 1.37E−01 | 3.19E−02 |
| 591 | 292.V.L | 1.37E−01 | 2.76E−02 |
| 534 | 224.G.H | 1.36E−01 | 2.24E−02 |
| 791 | 953.—.K | 1.34E−01 | 2.60E−02 |
| 781 | 390.K.E | 1.33E−01 | 2.71E−02 |
| 718 | 7.I.L | 1.32E−01 | 2.85E−02 |
| 812 | 329.G.K | 1.29E−01 | 2.97E−02 |
| 609 | 405.L.N | 1.26E−01 | 2.57E−02 |
| 758 | 791.E.N | 1.24E−01 | 2.33E−02 |
| 616 | 414.—.Y | 1.22E−01 | 2.44E−02 |
| 632 | 469.L.S | 1.22E−01 | 2.79E−02 |
| 614 | 414.—.R | 1.18E−01 | 2.32E−02 |
| 721 | 156.F.V | 1.14E−01 | 2.48E−02 |
| 611 | 408.E.Y | 1.11E−01 | 2.08E−02 |
| 622 | 420.D.G | 1.07E−01 | 2.41E−02 |
| 610 | 405.L.W | 1.06E−01 | 2.21E−02 |
| 619 | 417.K.D | 1.05E−01 | 2.59E−02 |
| 580 | 86.W.D | 1.00E−01 | 2.22E−02 |
| 602 | 339.—.Q | 9.82E−02 | 2.53E−02 |
| 537 | 224.G.A | 9.21E−02 | 2.34E−02 |
| 587 | 224.G.— | 8.18E−02 | 2.19E−02 |
| 538 | 224.G.V | 7.16E−02 | 2.16E−02 |
| 702 | 91.K.V | 6.31E−02 | 1.93E−02 |
| 824 | 611.K.Q | 4.07E−02 | 1.23E−02 |
| 631 | 469.L.K | 3.01E−02 | 1.30E−02 |
| 573 | 53.E.P | 2.65E−02 | 1.11E−02 |
| 528 | 224.G.Y | 8.98E−03 | 7.97E−03 |
| 535 | 224.G.S | 7.92E−03 | 7.31E−03 |
| Cas9 | n/a | 7.17E−03 | 8.11E−03 |

*Positions of mutations are shown relative to a CasX 515 sequence with an N-terminal methionine residue (i.e., SEQ ID NO: 5, with the addition of an N-terminal methionine).

As shown in Table 48, CasX proteins 607, 532, 676, 592, 788, 583, and 555 produced higher levels of on-target editing than did CasX 515. CasX proteins 569, 787, 561, 577, 585, and 572 also produced relatively high levels of on-target editing, with at least 90% of the activity of CasX 515 (i.e., greater than 1.88E-01 on-target editing).

Table 49 provides the level of off-target editing produced by various CasX proteins with mutations relative to CasX 515, ranked from lowest to highest activity.

TABLE 49
Average off-targeting editing activity,
ranked from lowest to highest

| | | Average | SEM |
|---|---|---|---|
| | | off-target | off-target |
| Protein | Mutation relative | TTC PAM | TTC PAM |
| Designation | to CasX 515* | editing | editing |
| (CasX protein | (Position.Refer- | activity | activity |
| no., or Cas9) | ence.Alternative) | (fraction) | (fraction) |
| 528 | 224.G.Y | 1.73E−03 | 1.84E−03 |
| Cas9 | n/a | 2.18E−03 | 2.72E−03 |
| 535 | 224.G.S | 2.33E−03 | 2.63E−03 |
| 573 | 53.E.P | 5.01E−03 | 3.29E−03 |
| 824 | 611.K.Q | 5.86E−03 | 2.93E−03 |
| 631 | 469.L.K | 6.23E−03 | 3.45E−03 |
| 587 | 224.G.— | 1.15E−02 | 4.68E−03 |
| 538 | 224.G.V | 1.45E−02 | 7.45E−03 |
| 702 | 91.K.V | 1.52E−02 | 6.87E−03 |
| 812 | 329.G.K | 1.58E−02 | 7.16E−03 |
| 580 | 86.W.D | 1.78E−02 | 6.27E−03 |
| 619 | 417.K.D | 1.94E−02 | 6.80E−03 |
| 610 | 405.L.W | 2.08E−02 | 6.80E−03 |
| 758 | 791.E.N | 2.11E−02 | 7.51E−03 |
| 721 | 156.F.V | 2.12E−02 | 6.94E−03 |
| 591 | 292.V.L | 2.15E−02 | 7.81E−03 |
| 537 | 224.G.A | 2.18E−02 | 8.82E−03 |
| 590 | 289.K.S | 2.28E−02 | 8.23E−03 |
| 622 | 420.D.G | 2.30E−02 | 7.70E−03 |
| 632 | 469.L.S | 2.34E−02 | 7.92E−03 |
| 633 | 473.—.D | 2.36E−02 | 8.67E−03 |
| 614 | 414.—.R | 2.38E−02 | 7.57E−03 |
| 594 | 329.G.N | 2.41E−02 | 9.14E−03 |
| 643 | 593.Q.F | 2.46E−02 | 8.93E−03 |
| 609 | 405.L.N | 2.47E−02 | 8.24E−03 |
| 781 | 390.K.E | 2.48E−02 | 8.34E−03 |
| 616 | 414.—.Y | 2.48E−02 | 7.29E−03 |
| 602 | 339.—.Q | 2.55E−02 | 9.70E−03 |
| 791 | 953.—.K | 2.71E−02 | 7.83E−03 |
| 593 | 304.M.W | 2.87E−02 | 1.04E−02 |
| 644 | 593.Q.V | 2.88E−02 | 9.11E−03 |
| 657 | 893.—.N | 2.89E−02 | 1.02E−02 |
| 717 | 390.K.Q | 2.99E−02 | 1.00E−02 |
| 611 | 408.E.Y | 3.00E−02 | 8.86E−03 |
| 572 | 35.R.P | 3.06E−02 | 1.07E−02 |
| 780 | 389.—.V | 3.07E−02 | 9.83E−03 |
| 818 | 698.S.R | 3.15E−02 | 1.02E−02 |
| 638 | 481.E.D | 3.16E−02 | 1.01E−02 |
| 584 | 171.A.Y | 3.39E−02 | 1.07E−02 |
| 790 | 951.—.S | 3.47E−02 | 1.02E−02 |
| 718 | 7.I.L | 3.47E−02 | 1.06E−02 |
| 649 | 655.—.S | 3.54E−02 | 1.09E−02 |
| 562 | 5.K.G | 3.56E−02 | 1.09E−02 |
| 784 | 570.P.I | 3.60E−02 | 9.80E−03 |
| 736 | 794.P.A | 3.61E−02 | 1.03E−02 |
| 789 | 917.G.E | 3.63E−02 | 1.11E−02 |
| 544 | 232.D.G | 3.64E−02 | 1.15E−02 |
| 612 | 412.G.P | 3.64E−02 | 1.12E−02 |
| 604 | 342.—.A | 3.66E−02 | 1.11E−02 |
| 564 | 6.—.G | 3.67E−02 | 1.07E−02 |
| 568 | 8.N.S | 3.91E−02 | 1.15E−02 |
| 779 | 372.G.I | 3.98E−02 | 1.20E−02 |
| 760 | 797.T.V | 3.99E−02 | 1.09E−02 |
| 777 | 169.L.Q | 4.11E−02 | 1.16E−02 |
| 566 | 7.I.A | 4.20E−02 | 1.15E−02 |
| 569 | 9.K.G | 4.29E−02 | 1.03E−02 |
| 577 | 64.R.Q | 4.38E−02 | 1.28E−02 |
| 536 | 224.G.T | 4.44E−02 | 1.32E−02 |
| 656 | 887.T.D | 4.54E−02 | 1.22E−02 |
| 646 | 653.—.T | 4.56E−02 | 1.21E−02 |
| 757 | 796.K.Q | 4.60E−02 | 1.25E−02 |
| 559 | 4.I.G | 4.71E−02 | 1.19E−02 |
| 585 | 171.A.S | 4.73E−02 | 1.24E−02 |
| 515 | — | 4.85E−02 | 1.33E−02 |
| 762 | 793.—.P | 4.91E−02 | 1.25E−02 |
| 561 | 5.—.G | 4.92E−02 | 1.37E−02 |
| 788 | 891.S.Q | 5.50E−02 | 1.45E−02 |
| 555 | 171.A.D | 5.53E−02 | 1.52E−02 |
| 787 | 826.V.M | 5.68E−02 | 1.33E−02 |
| 654 | 772.M.S | 5.84E−02 | 1.61E−02 |
| 583 | 169.L.K | 5.95E−02 | 1.48E−02 |
| 592 | 304.M.T | 5.97E−02 | 1.48E−02 |
| 534 | 224.G.H | 6.37E−02 | 1.52E−02 |
| 676 | 27.—.R + | 7.13E−02 | 1.68E−02 |
| | 170.L.K + | | |
| | 224.G.S | | |
| 607 | 398.Y.T | 8.25E−02 | 1.82E−02 |
| 532 | 27.—.R | 8.70E−02 | 1.73E−02 |

*Positions of mutations are shown relative to a CasX 515 sequence with an N-terminal methionine residue (i.e., SEQ ID NO: 5, with the addition of an N-terminal methionine).

As shown in Table 49, many of the tested CasX proteins showed lower levels of off-target editing than did CasX 515. For example, consistent with previous results, CasX 812 produced relatively low levels of off-target editing. Further, some of the tested CasX proteins showed even lower levels of off-target editing than did CasX 812 (specifically, CasX 528, 535, 573, 824, 631, 587, 538, and 702).

Based on these results, a set of mutation conferring a high degree of editing activity and/or specificity was chosen for introducing in pairs into CasX 515. First, high activity mutations were defined as those that showed a level of on-target editing equal to at least 87.3% of the level of on-target editing by CasX 515. CasX 607, 532, 676, 592, 788, 583, 555, 569, 787, 561, 577, 585, 572, 536, 656, 559, 777, and 584 met this threshold, and were therefore selected as potential activity-enhancing mutations (see Table 50). Second, high specificity mutations were defined as those producing 80% or lower of the level of off-target editing produced by CasX 515, while maintaining at least 79.95% of the on-target editing activity of CasX 515. This 80% on-target editing activity requirement was implemented to avoid selecting mutations that were simply loss-of-function mutations and would therefore not be expected to be useful as gene editors. CasX 593, 572, 818, 638, 584, 562, and 784 met these criteria, and were therefore selected as potential specificity-enhancing mutations (see Table 50).

In total, 22 individual mutations were chosen as candidates for introducing in pairs into CasX 515 and testing for improved properties, as described in Example 26, below. The positions of the individual mutations relative to full-length CasX 515 protein, as well as amino acid sequences of full-length CasX proteins with the individual mutations, are provided in Table 50. Table 51, below, shows the amino acid sequences and coordinates of the domains of CasX 515, and Table 52 shows the positions of the 22 individual mutations within the domains of CasX 515, as well as the amino acid sequences of domains with each individual mutation.

TABLE 50
Summary of positions of single mutations
within the CasX 515 protein

| | | Mutation relative | Full-length |
|---|---|---|---|
| CasX | | to CasX 515* | CasX amino |
| protein | | (Position.Refer- | acid sequence |
| no. | Phenotype | ence.Alternative) | (SEQ ID NO) |
| 532 | Activity | 27.—.R | 45 |
| 536 | Activity | 224.G.T | 49 |
| 555 | Activity | 171.A.D | 68 |
| 559 | Activity | 4.I.G | 72 |
| 561 | Activity | 5.—.G | 74 |
| 562 | Specificity | 5.K.G | 75 |
| 564 | Specificity | 6.—.G | 77 |
| 569 | Activity | 9.K.G | 82 |
| 572 | Activity | 35.R.P | 85 |
| | (and specificity) | | |
| 577 | Activity | 64.R.Q | 90 |
| 583 | Activity | 169.L.K | 96 |
| 584 | Activity | 171.A.Y | 97 |
| | (and specificity) | | |
| 585 | Activity | 171.A.S | 98 |
| 592 | Activity | 304.M.T | 104 |
| 593 | Specificity | 304.M.W | 105 |
| 607 | Activity | 398.Y.T | 119 |
| 638 | Specificity | 481.E.D | 150 |
| 656 | Activity | 887.T.D | 167 |
| 777 | Activity | 169.L.Q | 283 |
| 787 | Activity | 826.V.M | 293 |
| 788 | Activity | 891.S.Q | 294 |
| 818 | Specificity | 698.S.R | 322 |

*Positions of mutations are shown relative to a CasX 515 sequence with an N-terminal methionine residue (i.e., SEQ ID NO: 5, with the addition of an N-terminal methionine).

TABLE 51
CasX 515 domain sequences and coordinates

| | SEQ | | |
|---|---|---|---|
| Domain | ID NO | Amino Acid Sequence | Coordinates |
| N-terminal | — | M | 1 |
| methionine | | | |
| OBD-I | 352 | QEIKRINKIRRRLVKDSNTKKAGKTGPMKTLLVRVMTPDLRERLENLR | 2-57 |
| | | KKPENIPQ | |
| Helical I-I | 353 | PISNTSRANLNKLLTDYTEMKKAILHVYWEEFQKDPVGLMSRVA | 58-101 |
| NTSB | 345 | QPASKKIDQNKLKPEMDEKGNLTTAGFACSQCGQPLFVYKLEQVSEKG | 102-192 |
| | | KAYTNYFGRCNVAEHEKLILLAQLKPEKDSDEAVTYSLGKFGQ | |
| Helical I-II | 14332 | RALDFYSIHVTKESTHPVKPLAQIAGNRYASGPVGKALSDACMGTIAS | 193-333 |
| | | FLSKYQDIIIEHQKVVKGNQKRLESLRELAGKENLEYPSVTLPPQPHT | |
| | | KEGVDAYNEVIARVRMWVNLNLWQKLKLSRDDAKPLLRLKGFPSF | |
| Helical II | 14333 | PLVERQANEVDWWDMVCNVKKLINEKKEDGKVFWQNLAGYKRQEALRP | 334-501 |
| | | YLSSEEDRKKGKKFARYQLGDLLLHLEKKHGEDWGKVYDEAWERIDKK | |
| | | VEGLSKHIKLEEERRSEDAQSKAALTDWLRAKASFVIEGLKEADKDEF | |
| | | CRCELKLQKWYGDLRGKPFAIEAE | |
| OBD-II | 357 | NSILDISGFSKQYNCAFIWQKDGVKKLNLYLIINYFKGGKLRFKKIKP | 502-647 |
| | | EAFEANRFYTVINKKSGEIVPMEVNENFDDPNLIILPLAFGKRQGREF | |
| | | IWNDLLSLETGSLKLANGRVIEKTLYNRRTRQDEPALFVALTFERREV | |
| | | LD | |
| RuvC-I | 14334 | SSNIKPMNLIGVDRGENIPAVIALTDPEGCPLSRFKDSLGNPTHILRI | 648-811 |
| | | GESYKEKQRTIQAKKEVEQRRAGGYSRKYASKAKNLADDMVRNTARDL | |
| | | LYYAVTQDAMLIFENLSRGFGRQGKRTEMAERQYTRMEDWLTAKLAYE | |
| | | GLPSKTYLSKTLAQYTSKTC | |
| TSL | 359 | SNCGFTITSADYDRVLEKLKKTATGWMTTINGKELKVEGQITYYNRYK | 812-921 |
| | | RQNVVKDLSVELDRLSEESVNNDISSWTKGRSGEALSLLKKRFSHRPV | |
| | | QEKFVCLNCGFETH | |
| RuvC-II | 360 | ADEQAALNIARSWLFLRSQEYKKYQTNKTTGNTDKRAFVETWQSFYRK | 922-979 |
| | | KLKEVWKPAV | |

TABLE 52
Summary of positions of single mψtations within CasX 515 protein domains

| | | Position of | | |
|---|---|---|---|---|
| | | mψtation within | | Amino acid |
| | | CasX 515 domain* | | sequence of |
| CasX | | (Position. | | mψtated |
| protein | Mutated | Reference. | Amino acid sequence of mψtated | domain |
| no. | domain | Alternative) | domaint | (SEQ ID NO) |
| 532 | OBD-I | 26.—.R | QEIKRINKIRRRLVKDSNTKKAGKTRGPMK | 14287 |
| | | | TLLVRVMTPDLRERLENLRKKPENIPQ | |
| 536 | Helical I-II | 32.G.T | RALDFYSIHVTKESTHPVKPLAQIAGNRYA | 14288 |
| | | | STPVGKALSDACMGTIASFLSKYQDIIIEH | |
| | | | QKVVKGNQKRLESLRELAGKENLEYPSVTL | |
| | | | PPQPHTKEGVDAYNEVIARVRMWVNLNLWQ | |
| | | | KLKLSRDDAKPLLRLKGFPSF | |
| 555 | NTSB | 70.A.D | QPASKKIDQNKLKPEMDEKGNLTTAGFACS | 14289 |
| | | | QCGQPLFVYKLEQVSEKGKAYTNYFGRCNV | |
| | | | AEHEKLILLDQLKPEKDSDEAVTYSLGKFG | |
| | | | Q | |
| 559 | OBD-I | 3.I.G | QEGKRINKIRRRLVKDSNTKKAGKTGPMKT | 14290 |
| | | | LLVRVMTPDLRERLENLRKKPENIPQ | |
| 561 | OBD-I | 4.—.G | QEIGKRINKIRRRLVKDSNTKKAGKTGPMK | 14291 |
| | | | TLLVRVMTPDLRERLENLRKKPENIPQ | |
| 562 | OBD-I | 4.K.G | QEIGRINKIRRRLVKDSNTKKAGKTGPMKT | 14292 |
| | | | LLVRVMTPDLRERLENLRKKPENIPQ | |
| 564 | OBD-I | 5.—.G | QEIKGRINKIRRRLVKDSNTKKAGKTGPMK | 14293 |
| | | | TLLVRVMTPDLRERLENLRKKPENIPQ | |
| 569 | OBD-I | 8.K.G | QEIKRINGIRRRLVKDSNTKKAGKTGPMKT | 14294 |
| | | | LLVRVMTPDLRERLENLRKKPENIPQ | |
| 572 | OBD-I | 34.R.P | QEIKRINKIRRRLVKDSNTKKAGKTGPMKT | 14295 |
| | | | LLVPVMTPDLRERLENLRKKPENIPQ | |
| 577 | Helical I-I | 7.R.Q | PISNTSQANLNKLLTDYTEMKKAILHVYWE | 14296 |
| | | | EFQKDPVGLMSRVA | |
| 583 | NTSB | 68.L.K | QPASKKIDQNKLKPEMDEKGNLTTAGFACS | 14297 |
| | | | QCGQPLFVYKLEQVSEKGKAYTNYFGRCNV | |
| | | | AEHEKLIKLAQLKPEKDSDEAVTYSLGKFG | |
| | | | Q | |
| 584 | NTSB | 70.A.Y | QPASKKIDQNKLKPEMDEKGNLTTAGFACS | 14298 |
| | | | QCGQPLFVYKLEQVSEKGKAYTNYFGRCNV | |
| | | | AEHEKLILLYQLKPEKDSDEAVTYSLGKFG | |
| | | | Q | |
| 585 | NTSB | 70.A.S | QPASKKIDQNKLKPEMDEKGNLTTAGFACS | 14299 |
| | | | QCGQPLFVYKLEQVSEKGKAYTNYFGRCNV | |
| | | | AEHEKLILLSQLKPEKDSDEAVTYSLGKFG | |
| | | | Q | |
| 592 | Helical I-II | 112.M.T | RALDFYSIHVTKESTHPVKPLAQIAGNRYA | 14300 |
| | | | SGPVGKALSDACMGTIASFLSKYQDIIIEH | |
| | | | QKVVKGNQKRLESLRELAGKENLEYPSVTL | |
| | | | PPQPHTKEGVDAYNEVIARVRTWVNLNLWQ | |
| | | | KLKLSRDDAKPLLRLKGFPSF | |
| 593 | Helical I-II | 112.M.W | RALDFYSIHVTKESTHPVKPLAQIAGNRYA | 14301 |
| | | | SGPVGKALSDACMGTIASFLSKYQDIIIEH | |
| | | | QKVVKGNQKRLESLRELAGKENLEYPSVTL | |
| | | | PPQPHTKEGVDAYNEVIARVRWWVNLNLWQ | |
| | | | KLKLSRDDAKPLLRLKGFPSF | |
| 607 | Helical II | 65.Y.T | PLVERQANEVDWWDMVCNVKKLINEKKEDG | 14302 |
| | | | KVFWQNLAGYKRQEALRPYLSSEEDRKKGK | |
| | | | KFARTQLGDLLLHLEKKHGEDWGKVYDEAW | |
| | | | ERIDKKVEGLSKHIKLEEERRSEDAQSKAA | |
| | | | LTDWLRAKASFVIEGLKEADKDEFCRCELK | |
| | | | LQKWYGDLRGKPFAIEAE | |
| 638 | Helical II | 148.E.D | PLVERQANEVDWWDMVCNVKKLINEKKEDG | 14303 |
| | | | KVFWQNLAGYKRQEALRPYLSSEEDRKKGK | |
| | | | KFARYQLGDLLLHLEKKHGEDWGKVYDEAW | |
| | | | ERIDKKVEGLSKHIKLEEERRSEDAQSKAA | |
| | | | LTDWLRAKASFVIEGLKEADKDEFCRCDLK | |
| | | | LQKWYGDLRGKPFAIEAE | |
| 656 | TSL | 76.T.D | SNCGFTITSADYDRVLEKLKKTATGWMTTI | 14304 |
| | | | NGKELKVEGQITYYNRYKRQNVVKDLSVEL | |
| | | | DRLSEESVNNDISSWDKGRSGEALSLLKKR | |
| | | | FSHRPVQEKFVCLNCGFETH | |
| 777 | NTSB | 68.L.Q | QPASKKIDQNKLKPEMDEKGNLTTAGFACS | 14305 |
| | | | QCGQPLFVYKLEQVSEKGKAYTNYFGRCNV | |
| | | | AEHEKLIQLAQLKPEKDSDEAVTYSLGKFG | |
| 787 | TSL | 15.V.M | SNCGFTITSADYDRMLEKLKKTATGWMTTI | 14306 |
| | | | NGKELKVEGQITYYNRYKRQNVVKDLSVEL | |
| | | | DRLSEESVNNDISSWTKGRSGEALSLLKKR | |
| | | | FSHRPVQEKFVCLNCGFETH | |
| 788 | TSL | 80.S.Q | SNCGFTITSADYDRVLEKLKKTATGWMTTI | 14307 |
| | | | NGKELKVEGQITYYNRYKRQNVVKDLSVEL | |
| | | | DRLSEESVNNDISSWTKGRQGEALSLLKKR | |
| | | | FSHRPVQEKFVCLNCGFETH | |
| 818 | RuvC-I | 51.S.R | SSNIKPMNLIGVDRGENIPAVIALTDPEGC | 14308 |
| | | | PLSRFKDSLGNPTHILRIGERYKEKQRTIQ | |
| | | | AKKEVEQRRAGGYSRKYASKAKNLADDMVR | |
| | | | NTARDLLYYAVTQDAMLIFENLSRGFGRQG | |
| | | | KRTFMAERQYTRMEDWLTAKLAYEGLPSKT | |
| | | | YLSKTLAQYTSKTC | |

*Positions of mψtations within domains are shown relative to the CasX 515 domain sequences provided in Table 51, above.
+Mutated residues are bolded and underlined.

Example 26: Engineered CasX Proteins with Pairs of Mutations Relative to CasX 515

Engineered CasX proteins were generated with pairs of mutations relative to CasX 515, and assessed for their on and off-target gene editing activity.

### Materials and Methods

Pairs of mutations listed in Tables 50 and 52, above, were introduced into the CasX 515 amino acid sequence to generate 161 amino acid sequences of engineered CasX proteins. The pairs of mutations and full-length amino acid sequences of the 161 engineered CasX proteins tested are listed in Tables 53, and Table 54 provides the amino acid sequences of each of the domains of the 161 engineered CasX proteins.

TABLE 53
Pairs of mutations and amino acid sequences
of engineered CasX proteins

| Mutations relative | | Full-length |
|---|---|---|
| to CasX 515* | Phenotypes of | CasX |
| (Position.Refer- | single mutations | amino acid |
| ence.Alternative) | (see Example 25) | SEQ ID NO |
| 4.I.G & 64.R.Q | Activity + activity | 14126 |
| 4.I.G & 169.L.K | Activity + activity | 14127 |
| 4.I.G & 169.L.Q | Activity + activity | 14128 |
| 4.I.G & 171.A.D | Activity + activity | 14129 |
| 4.I.G & 171.A.Y | Activity + activity | 14130 |
| 4.I.G & 171.A.S | Activity + activity | 14131 |
| 4.I.G & 224.G.T | Activity + activity | 14132 |
| 4.I.G & 304.M.T | Activity + activity | 14133 |
| 4.I.G & 398.Y.T | Activity + activity | 14134 |
| 4.I.G & 826.V.M | Activity + activity | 14135 |
| 4.I.G & 887.T.D | Activity + activity | 14136 |
| 4.I.G & 891.S.Q | Activity + activity | 14137 |
| 5.—.G & 64.R.Q | Activity + activity | 14138 |
| 5.—.G & 169.L.K | Activity + activity | 14139 |
| 5.—.G & 169.L.Q | Activity + activity | 14140 |
| 5.—.G & 171.A.D | Activity + activity | 14141 |
| 5.—.G & 171.A.Y | Activity + activity | 14142 |
| 5.— G & 171.A.S | Activity + activity | 14143 |
| 5.—.G & 224.G.T | Activity + activity | 14144 |
| 5.—.G & 304.M.T | Activity + activity | 14145 |
| 5.—.G & 398.Y.T | Activity + activity | 14146 |
| 5.—.G & 826.V.M | Activity + activity | 14147 |
| 5.—.G & 887.T.D | Activity + activity | 14148 |
| 5.—.G & 891.S.Q | Activity + activity | 14149 |
| 9.K.G & 64.R.Q | Activity + activity | 14150 |
| 9.K.G & 169.L.K | Activity + activity | 14151 |
| 9.K.G & 169.L.Q | Activity + activity | 14152 |
| 9.K.G & 171.A.D | Activity + activity | 14153 |
| 9.K.G & 171.A.Y | Activity + activity | 14154 |
| 9.K.G & 171.A.S | Activity + activity | 14155 |
| 9.K.G & 224.G.T | Activity + activity | 14156 |
| 9.K.G & 304.M.T | Activity + activity | 14157 |
| 9.K.G & 398.Y.T | Activity + activity | 14158 |
| 9.K.G & 826.V.M | Activity + activity | 14159 |
| 9.K.G & 887.T.D | Activity + activity | 14160 |
| 9.K.G & 891.S.Q | Activity + activity | 14161 |
| 27.—.R & 64.R.Q | Activity + activity | 14162 |
| 27.—.R & 169.L.K | Activity + activity | 14163 |
| 27.—.R & 169.L.Q | Activity + activity | 14164 |
| 27.—.R & 171.A.D | Activity + activity | 14165 |
| 27.—.R & 171.A.Y | Activity + activity | 14166 |
| 27.—.R & 171.A.S | Activity + activity | 14167 |
| 27.—.R & 224.G.T | Activity + activity | 14168 |
| 27.—.R & 304.M.T | Activity + activity | 14169 |
| 27.—.R & 398.Y.T | Activity + activity | 14170 |
| 27.—.R & 826.V.M | Activity + activity | 14171 |
| 27.—.R & 887.T.D | Activity + activity | 14172 |
| 27.—.R & 891.S.Q | Activity + activity | 14173 |
| 35.R.P & 64.R.Q | Activity + activity | 14174 |
| 35.R.P & 169.L.K | Activity + activity | 14175 |
| 35.R.P & 169.L.Q | Activity + activity | 14176 |
| 35.R.P & 171.A.D | Activity + activity | 14177 |
| 35.R.P & 171.A.Y | Activity + activity | 14178 |
| 35.R.P & 171.A.S | Activity + activity | 14179 |
| 35.R.P & 224.G.T | Activity + activity | 14180 |
| 35.R.P & 304.M.T | Activity + activity | 14181 |
| 35.R.P & 398.Y.T | Activity + activity | 14182 |
| 35.R.P & 826.V.M | Activity + activity | 14183 |
| 35.R.P & 887.T.D | Activity + activity | 14184 |
| 35.R.P & 891.S.Q | Activity + activity | 14185 |
| 887.T.D & 891.S.Q | Activity + activity | 14186 |
| 64.R.Q & 169.L.K | Activity + activity | 14187 |
| 64.R.Q & 169.L.Q | Activity + activity | 14188 |
| 64.R.Q & 171.A.D | Activity + activity | 14189 |
| 64.R.Q & 171.A.Y | Activity + activity | 14190 |
| 64.R.Q & 171.A.S | Activity + activity | 14191 |
| 64.R.Q & 224.G.T | Activity + activity | 14192 |
| 64.R.Q & 304.M.T | Activity + activity | 14193 |
| 64.R.Q & 398.Y.T | Activity + activity | 14194 |
| 64.R.Q & 826.V.M | Activity + activity | 14195 |
| 64.R.Q & 887.T.D | Activity + activity | 14196 |
| 64.R.Q & 891.S.Q | Activity + activity | 14197 |
| 169.L.K & 171.A.D | Activity + activity | 14198 |
| 169.L.K & 171.A.Y | Activity + activity | 14199 |
| 169.L.K & 171.A.S | Activity + activity | 14200 |
| 169.L.K & 224.G.T | Activity + activity | 14201 |
| 169.L.K & 304.M.T | Activity + activity | 14202 |
| 169.L.K & 398.Y.T | Activity + activity | 14203 |
| 169.L.K & 826.V.M | Activity + activity | 14204 |
| 169.L.K & 887.T.D | Activity + activity | 14205 |
| 169.L.K & 891.S.Q | Activity + activity | 14206 |
| 169.L.Q & 171.A.D | Activity + activity | 14207 |
| 169.L.Q & 171.A.Y | Activity + activity | 14208 |
| 169.L.Q & 171.A.S | Activity + activity | 14209 |
| 169.L.Q & 224.G.T | Activity + activity | 14210 |
| 169.L.Q & 304.M.T | Activity + activity | 14211 |
| 169.L.Q & 398.Y.T | Activity + activity | 14212 |
| 169.L.Q & 826.V.M | Activity + activity | 14213 |
| 169.L.Q & 887.T.D | Activity + activity | 14214 |
| 169.L.Q & 891.S.Q | Activity + activity | 14215 |
| 171.A.D & 224.G.T | Activity + activity | 14216 |
| 171.A.D & 304.M.T | Activity + activity | 14217 |
| 171.A.D & 398.Y.T | Activity + activity | 14218 |
| 171.A.D & 826.V.M | Activity + activity | 14219 |
| 171.A.D & 887.T.D | Activity + activity | 14220 |
| 171.A.D & 891.S.Q | Activity + activity | 14221 |
| 171.A.Y & 224.G.T | Activity + activity | 14222 |
| 171.A.Y & 304.M.T | Activity + activity | 14223 |
| 171.A.Y & 398.Y.T | Activity + activity | 14224 |
| 171.A.Y & 826.V.M | Activity + activity | 14225 |
| 171.A.Y & 887.T.D | Activity + activity | 14226 |
| 171.A.Y & 891.S.Q | Activity + activity | 14227 |
| 171.A.S & 224.G.T | Activity + activity | 14228 |
| 171.A.S & 304.M.T | Activity + activity | 14229 |
| 171.A.S & 398.Y.T | Activity + activity | 14230 |
| 171.A.S & 826.V.M | Activity + activity | 14231 |
| 171.A.S & 887.T.D | Activity + activity | 14232 |
| 171.A.S & 891.S.Q | Activity + activity | 14233 |
| 4.I.G & 35.R.P | Activity + activity | 14234 |
| 224.G.T & 304.M.T | Activity + activity | 14235 |
| 224.G.T & 398.Y.T | Activity + activity | 14236 |
| 224.G.T & 826.V.M | Activity + activity | 14237 |
| 224.G.T & 887.T.D | Activity + activity | 14238 |
| 224.G.T & 891.S.Q | Activity + activity | 14239 |
| 5.—.G & 35.R.P | Activity + activity | 14240 |
| 4.I.G & 27.—.R | Activity + activity | 14241 |
| 304.M.T & 398.Y.T | Activity + activity | 14242 |
| 304.M.T & 826.V.M | Activity + activity | 14243 |
| 304.M.T & 887.T.D | Activity + activity | 14244 |
| 304.M.T & 891.S.Q | Activity + activity | 14245 |
| 9.K.G & 35.R.P | Activity + activity | 14246 |
| 5.—.G & 27.—.R | Activity + activity | 14247 |
| 4.I.G & 9.K.G | Activity + activity | 14248 |
| 398.Y.T & 826.V.M | Activity + activity | 14249 |
| 398.Y.T & 887.T.D | Activity + activity | 14250 |
| 398.Y.T & 891.S.Q | Activity + activity | 14251 |
| 27.—.R & 35.R.P | Activity + activity | 14252 |
| 9.K.G & 27.—.R | Activity + activity | 14253 |
| 5.—.G & 9.K.G | Activity + activity | 14254 |
| 4.I.G & 5.—.G | Activity + activity | 14255 |
| 826.V.M & 887.T.D | Activity + activity | 14256 |
| 826.V.M & 891.S.Q | Activity + activity | 14257 |
| 5.K.G & 27.—.R | Activity + specificity | 14258 |
| 5.K.G & 169.L.K | Activity + specificity | 14259 |
| 5.K.G & 171.A.D | Activity + specificity | 14260 |
| 5.K.G & 304.M.T | Activity + specificity | 14261 |
| 5.K.G & 398.Y.T | Activity + specificity | 14262 |
| 5.K.G & 891.S.Q | Activity + specificity | 14263 |
| 6.—.G & 27.—.R | Activity + specificity | 14264 |
| 6.—.G & 169.L.K | Activity + specificity | 14265 |
| 6.—.G & 171.A.D | Activity + specificity | 14266 |
| 6.—.G & 304.M.T | Activity + specificity | 14267 |
| 6.—.G & 398.Y.T | Activity + specificity | 14268 |
| 6.—.G & 891.S.Q | Activity + specificity | 14269 |
| 304.M.W & 27.—.R | Activity + specificity | 14270 |
| 304.M.W & 169.L.K | Activity + specificity | 14271 |
| 304.M.W & 171.A.D | Activity + specificity | 14272 |
| 304.M.W & 398.Y.T | Activity + specificity | 14273 |
| 304.M.W & 891.S.Q | Activity + specificity | 14274 |
| 481.E.D & 27.—.R | Activity + specificity | 14275 |
| 481.E.D & 169.L.K | Activity + specificity | 14276 |
| 481.E.D & 171.A.D | Activity + specificity | 14277 |
| 481.E.D & 304.M.T | Activity + specificity | 14278 |
| 481.E.D & 398.Y.T | Activity + specificity | 14279 |
| 481.E.D & 891.S.Q | Activity + specificity | 14280 |
| 698.S.R & 27.—.R | Activity + specificity | 14281 |
| 698.S.R & 169.L.K | Activity + specificity | 14282 |
| 698.S.R & 171.A.D | Activity + specificity | 14283 |
| 698.S.R & 304.M.T | Activity + specificity | 14284 |
| 698.S.R & 398.Y.T | Activity + specificity | 14285 |
| 698.S.R & 891.S.Q | Activity + specificity | 14286 |

*Positions of mutations are shown relative to a CasX 515 sequence with an N-terminal methionine residue (i.e., SEQ ID NO: 5, with the addition of an N-terminal methionine).

TABLE 54
Amino acid sequences of domains of engineered CasX proteins, N- to C-terminus

Mutations relative

| to CasX 515* | Amino acid sequences of domains of | | | | | | | | |
|---|---|---|---|---|---|---|---|---|---|
| (Position. | engineered CasX proteins (SEQ ID NOs) | | | | | | | | |
| Reference. | OBD- | Helical | | Helical | Helical | OBD- | RuvC- | | RuvC- |
| Alternative) | I | I-I | NTSB | I-II | II | II | I | TSL | II |
| None (non-mutated CasX 515) | 352 | 353 | 345 | 14332 | 14333 | 357 | 14334 | 359 | 360 |
| 4.I.G & 64.R.Q | 14290 | 14296 | 345 | 14332 | 14333 | 357 | 14334 | 359 | 360 |
| 4.I.G & 169.L.K | 14290 | 353 | 14297 | 14332 | 14333 | 357 | 14334 | 359 | 360 |
| 4.I.G & 169.L.Q | 14290 | 353 | 14305 | 14332 | 14333 | 357 | 14334 | 359 | 360 |
| 4.I.G & 171.A.D | 14290 | 353 | 14289 | 14332 | 14333 | 357 | 14334 | 359 | 360 |
| 4.I.G & 171.A.Y | 14290 | 353 | 14298 | 14332 | 14333 | 357 | 14334 | 359 | 360 |
| 4.I.G & 171.A.S | 14290 | 353 | 14299 | 14332 | 14333 | 357 | 14334 | 359 | 360 |
| 4.I.G & 224.G.T | 14290 | 353 | 345 | 14288 | 14333 | 357 | 14334 | 359 | 360 |
| 4.I.G & 304.M.T | 14290 | 353 | 345 | 14300 | 14333 | 357 | 14334 | 359 | 360 |
| 4.I.G & 398.Y.T | 14290 | 353 | 345 | 14332 | 14302 | 357 | 14334 | 359 | 360 |
| 4.I.G & 826.V.M | 14290 | 353 | 345 | 14332 | 14333 | 357 | 14334 | 14306 | 360 |
| 4.I.G & 887.T.D | 14290 | 353 | 345 | 14332 | 14333 | 357 | 14334 | 14304 | 360 |
| 4.I.G & 891.S.Q | 14290 | 353 | 345 | 14332 | 14333 | 357 | 14334 | 14307 | 360 |
| 5.—.G & 64.R.Q | 14291 | 14296 | 345 | 14332 | 14333 | 357 | 14334 | 359 | 360 |
| 5.—.G & 169.L.K | 14291 | 353 | 14297 | 14332 | 14333 | 357 | 14334 | 359 | 360 |
| 5.—.G & 169.L.Q | 14291 | 353 | 14305 | 14332 | 14333 | 357 | 14334 | 359 | 360 |
| 5.—.G & 171.A.D | 14291 | 353 | 14289 | 14332 | 14333 | 357 | 14334 | 359 | 360 |
| 5.—.G & 171.A.Y | 14291 | 353 | 14298 | 14332 | 14333 | 357 | 14334 | 359 | 360 |
| 5.—.G & 171.A.S | 14291 | 353 | 14299 | 14332 | 14333 | 357 | 14334 | 359 | 360 |
| 5.—.G & 224.G.T | 14291 | 353 | 345 | 14288 | 14333 | 357 | 14334 | 359 | 360 |
| 5.—.G & 304.M.T | 14291 | 353 | 345 | 14300 | 14333 | 357 | 14334 | 359 | 360 |
| 5.—.G & 398.Y.T | 14291 | 353 | 345 | 14332 | 14302 | 357 | 14334 | 359 | 360 |
| 5.—.G & 826.V.M | 14291 | 353 | 345 | 14332 | 14333 | 357 | 14334 | 14306 | 360 |
| 5.—.G & 887.T.D | 14291 | 353 | 345 | 14332 | 14333 | 357 | 14334 | 14304 | 360 |
| 5.—.G & 891.S.Q | 14291 | 353 | 345 | 14332 | 14333 | 357 | 14334 | 14307 | 360 |
| 9.K.G & 64.R.Q | 14294 | 14296 | 345 | 14332 | 14333 | 357 | 14334 | 359 | 360 |
| 9.K.G & 169.L.K | 14294 | 353 | 14297 | 14332 | 14333 | 357 | 14334 | 359 | 360 |
| 9.K.G & 169.L.Q | 14294 | 353 | 14305 | 14332 | 14333 | 357 | 14334 | 359 | 360 |
| 9.K.G & 171.A.D | 14294 | 353 | 14289 | 14332 | 14333 | 357 | 14334 | 359 | 360 |
| 9.K.G & 171.A.Y | 14294 | 353 | 14298 | 14332 | 14333 | 357 | 14334 | 359 | 360 |
| 9.K.G & 171.A.S | 14294 | 353 | 14299 | 14332 | 14333 | 357 | 14334 | 359 | 360 |
| 9.K.G & 224.G.T | 14294 | 353 | 345 | 14288 | 14333 | 357 | 14334 | 359 | 360 |
| 9.K.G & 304.M.T | 14294 | 353 | 345 | 14300 | 14333 | 357 | 14334 | 359 | 360 |
| 9.K.G & 398.Y.T | 14294 | 353 | 345 | 14332 | 14302 | 357 | 14334 | 359 | 360 |
| 9.K.G & 826.V.M | 14294 | 353 | 345 | 14332 | 14333 | 357 | 14334 | 14306 | 360 |
| 9.K.G & 887.T.D | 14294 | 353 | 345 | 14332 | 14333 | 357 | 14334 | 14304 | 360 |
| 9.K.G & 891.S.Q | 14294 | 353 | 345 | 14332 | 14333 | 357 | 14334 | 14307 | 360 |
| 27.—.R & 64.R.Q | 14287 | 14296 | 345 | 14332 | 14333 | 357 | 14334 | 359 | 360 |
| 27.—.R & 169.L.K | 14287 | 353 | 14297 | 14332 | 14333 | 357 | 14334 | 359 | 360 |
| 27.—.R & 169.L.Q | 14287 | 353 | 14305 | 14332 | 14333 | 357 | 14334 | 359 | 360 |
| 27.—.R & 171.A.D | 14287 | 353 | 14289 | 14332 | 14333 | 357 | 14334 | 359 | 360 |
| 27.—.R & 171.A.Y | 14287 | 353 | 14298 | 14332 | 14333 | 357 | 14334 | 359 | 360 |
| 27.—.R & 171.A.S | 14287 | 353 | 14299 | 14332 | 14333 | 357 | 14334 | 359 | 360 |
| 27.—.R & 224.G.T | 14287 | 353 | 345 | 14288 | 14333 | 357 | 14334 | 359 | 360 |
| 27.—.R & 304.M.T | 14287 | 353 | 345 | 14300 | 14333 | 357 | 14334 | 359 | 360 |
| 27.—.R & 398.Y.T | 14287 | 353 | 345 | 14332 | 14302 | 357 | 14334 | 359 | 360 |
| 27.—.R & 826.V.M | 14287 | 353 | 345 | 14332 | 14333 | 357 | 14334 | 14306 | 360 |
| 27.—.R & 887.T.D | 14287 | 353 | 345 | 14332 | 14333 | 357 | 14334 | 14304 | 360 |
| 27.—.R & 891.S.Q | 14287 | 353 | 345 | 14332 | 14333 | 357 | 14334 | 14307 | 360 |
| 35.R.P & 64.R.Q | 14295 | 14296 | 345 | 14332 | 14333 | 357 | 14334 | 359 | 360 |
| 35.R.P & 169.L.K | 14295 | 353 | 14297 | 14332 | 14333 | 357 | 14334 | 359 | 360 |
| 35.R.P & 169.L.Q | 14295 | 353 | 14305 | 14332 | 14333 | 357 | 14334 | 359 | 360 |
| 35.R.P & 171.A.D | 14295 | 353 | 14289 | 14332 | 14333 | 357 | 14334 | 359 | 360 |
| 35.R.P & 171.A.Y | 14295 | 353 | 14298 | 14332 | 14333 | 357 | 14334 | 359 | 360 |
| 35.R.P & 171.A.S | 14295 | 353 | 14299 | 14332 | 14333 | 357 | 14334 | 359 | 360 |
| 35.R.P & 224.G.T | 14295 | 353 | 345 | 14288 | 14333 | 357 | 14334 | 359 | 360 |
| 35.R.P & 304.M.T | 14295 | 353 | 345 | 14300 | 14333 | 357 | 14334 | 359 | 360 |
| 35.R.P & 398.Y.T | 14295 | 353 | 345 | 14332 | 14302 | 357 | 14334 | 359 | 360 |
| 35.R.P & 826.V.M | 14295 | 353 | 345 | 14332 | 14333 | 357 | 14334 | 14306 | 360 |
| 35.R.P & 887.T.D | 14295 | 353 | 345 | 14332 | 14333 | 357 | 14334 | 14304 | 360 |
| 35.R.P & 891.S.Q | 14295 | 353 | 345 | 14332 | 14333 | 357 | 14334 | 14307 | 360 |
| 887.T.D & 891.S.Q | 352 | 353 | 345 | 14332 | 14333 | 357 | 14334 | 14329 | 360 |
| 64.R.Q & 169.L.K | 352 | 14296 | 14297 | 14332 | 14333 | 357 | 14334 | 359 | 360 |
| 64.R.Q & 169.L.Q | 352 | 14296 | 14305 | 14332 | 14333 | 357 | 14334 | 359 | 360 |
| 64.R.Q & 171.A.D | 352 | 14296 | 14289 | 14332 | 14333 | 357 | 14334 | 359 | 360 |
| 64.R.Q & 171.A.Y | 352 | 14296 | 14298 | 14332 | 14333 | 357 | 14334 | 359 | 360 |
| 64.R.Q & 171.A.S | 352 | 14296 | 14299 | 14332 | 14333 | 357 | 14334 | 359 | 360 |
| 64.R.Q & 224.G.T | 352 | 14296 | 345 | 14288 | 14333 | 357 | 14334 | 359 | 360 |
| 64.R.Q & 304.M.T | 352 | 14296 | 345 | 14300 | 14333 | 357 | 14334 | 359 | 360 |
| 64.R.Q & 398.Y.T | 352 | 14296 | 345 | 14332 | 14302 | 357 | 14334 | 359 | 360 |
| 64.R.Q & 826.V.M | 352 | 14296 | 345 | 14332 | 14333 | 357 | 14334 | 14306 | 360 |
| 64.R.Q & 887.T.D | 352 | 14296 | 345 | 14332 | 14333 | 357 | 14334 | 14304 | 360 |
| 64.R.Q & 891.S.Q | 352 | 14296 | 345 | 14332 | 14333 | 357 | 14334 | 14307 | 360 |
| 169.L.K & 171.A.D | 352 | 353 | 14321 | 14332 | 14333 | 357 | 14334 | 359 | 360 |
| 169.L.K & 171.A.Y | 352 | 353 | 14322 | 14332 | 14333 | 357 | 14334 | 359 | 360 |
| 169.L.K & 171.A.S | 352 | 353 | 14323 | 14332 | 14333 | 357 | 14334 | 359 | 360 |
| 169.L.K & 224.G.T | 352 | 353 | 14297 | 14288 | 14333 | 357 | 14334 | 359 | 360 |
| 169.L.K & 304.M.T | 352 | 353 | 14297 | 14300 | 14333 | 357 | 14334 | 359 | 360 |
| 169.L.K & 398.Y.T | 352 | 353 | 14297 | 14332 | 14302 | 357 | 14334 | 359 | 360 |
| 169.L.K & 826.V.M | 352 | 353 | 14297 | 14332 | 14333 | 357 | 14334 | 14306 | 360 |
| 169.L.K & 887.T.D | 352 | 353 | 14297 | 14332 | 14333 | 357 | 14334 | 14304 | 360 |
| 169.L.K & 891.S.Q | 352 | 353 | 14297 | 14332 | 14333 | 357 | 14334 | 14307 | 360 |
| 169.L.Q & 171.A.D | 352 | 353 | 14324 | 14332 | 14333 | 357 | 14334 | 359 | 360 |
| 169.L.Q & 171.A.Y | 352 | 353 | 14325 | 14332 | 14333 | 357 | 14334 | 359 | 360 |
| 169.L.Q & 171.A.S | 352 | 353 | 14326 | 14332 | 14333 | 357 | 14334 | 359 | 360 |
| 169.L.Q & 224.G.T | 352 | 353 | 14305 | 14288 | 14333 | 357 | 14334 | 359 | 360 |
| 169.L.Q & 304.M.T | 352 | 353 | 14305 | 14300 | 14333 | 357 | 14334 | 359 | 360 |
| 169.L.Q & 398.Y.T | 352 | 353 | 14305 | 14332 | 14302 | 357 | 14334 | 359 | 360 |
| 169.L.Q & 826.V.M | 352 | 353 | 14305 | 14332 | 14333 | 357 | 14334 | 14306 | 360 |
| 169.L.Q & 887.T.D | 352 | 353 | 14305 | 14332 | 14333 | 357 | 14334 | 14304 | 360 |
| 169.L.Q & 891.S.Q | 352 | 353 | 14305 | 14332 | 14333 | 357 | 14334 | 14307 | 360 |
| 171.A.D & 224.G.T | 352 | 353 | 14289 | 14288 | 14333 | 357 | 14334 | 359 | 360 |
| 171.A.D & 304.M.T | 352 | 353 | 14289 | 14300 | 14333 | 357 | 14334 | 359 | 360 |
| 171.A.D & 398.Y.T | 352 | 353 | 14289 | 14332 | 14302 | 357 | 14334 | 359 | 360 |
| 171.A.D & 826.V.M | 352 | 353 | 14289 | 14332 | 14333 | 357 | 14334 | 14306 | 360 |
| 171.A.D & 887.T.D | 352 | 353 | 14289 | 14332 | 14333 | 357 | 14334 | 14304 | 360 |
| 171.A.D & 891.S.Q | 352 | 353 | 14289 | 14332 | 14333 | 357 | 14334 | 14307 | 360 |
| 171.A.Y & 224.G.T | 352 | 353 | 14298 | 14288 | 14333 | 357 | 14334 | 359 | 360 |
| 171.A.Y & 304.M.T | 352 | 353 | 14298 | 14300 | 14333 | 357 | 14334 | 359 | 360 |
| 171.A.Y & 398.Y.T | 352 | 353 | 14298 | 14332 | 14302 | 357 | 14334 | 359 | 360 |
| 171.A.Y & 826.V.M | 352 | 353 | 14298 | 14332 | 14333 | 357 | 14334 | 14306 | 360 |
| 171.A.Y & 887.T.D | 352 | 353 | 14298 | 14332 | 14333 | 357 | 14334 | 14304 | 360 |
| 171.A.Y & 891.S.Q | 352 | 353 | 14298 | 14332 | 14333 | 357 | 14334 | 14307 | 360 |
| 171.A.S & 224.G.T | 352 | 353 | 14299 | 14288 | 14333 | 357 | 14334 | 359 | 360 |
| 171.A.S & 304.M.T | 352 | 353 | 14299 | 14300 | 14333 | 357 | 14334 | 359 | 360 |
| 171.A.S & 398.Y.T | 352 | 353 | 14299 | 14332 | 14302 | 357 | 14334 | 359 | 360 |
| 171.A.S & 826.V.M | 352 | 353 | 14299 | 14332 | 14333 | 357 | 14334 | 14306 | 360 |
| 171.A.S & 887.T.D | 352 | 353 | 14299 | 14332 | 14333 | 357 | 14334 | 14304 | 360 |
| 171.A.S & 891.S.Q | 352 | 353 | 14299 | 14332 | 14333 | 357 | 14334 | 14307 | 360 |
| 4.I.G & 35.R.P | 14309 | 353 | 345 | 14332 | 14333 | 357 | 14334 | 359 | 360 |
| 224.G.T & 304.M.T | 352 | 353 | 345 | 14327 | 14333 | 357 | 14334 | 359 | 360 |
| 224.G.T & 398.Y.T | 352 | 353 | 345 | 14288 | 14302 | 357 | 14334 | 359 | 360 |
| 224.G.T & 826.V.M | 352 | 353 | 345 | 14288 | 14333 | 357 | 14334 | 14306 | 360 |
| 224.G.T & 887.T.D | 352 | 353 | 345 | 14288 | 14333 | 357 | 14334 | 14304 | 360 |
| 224.G.T & 891.S.Q | 352 | 353 | 345 | 14288 | 14333 | 357 | 14334 | 14307 | 360 |
| 5.—.G & 35.R.P | 14310 | 353 | 345 | 14332 | 14333 | 357 | 14334 | 359 | 360 |
| 4.I.G & 27.—.R | 14311 | 353 | 345 | 14332 | 14333 | 357 | 14334 | 359 | 360 |
| 304.M.T & 398.Y.T | 352 | 353 | 345 | 14300 | 14302 | 357 | 14334 | 359 | 360 |
| 304.M.T & 826.V.M | 352 | 353 | 345 | 14300 | 14333 | 357 | 14334 | 14306 | 360 |
| 304.M.T & 887.T.D | 352 | 353 | 345 | 14300 | 14333 | 357 | 14334 | 14304 | 360 |
| 304.M.T & 891.S.Q | 352 | 353 | 345 | 14300 | 14333 | 357 | 14334 | 14307 | 360 |
| 9.K.G & 35.R.P | 14312 | 353 | 345 | 14332 | 14333 | 357 | 14334 | 359 | 360 |
| 5.—.G & 27.—.R | 14313 | 353 | 345 | 14332 | 14333 | 357 | 14334 | 359 | 360 |
| 4.I.G & 9.K.G | 14314 | 353 | 345 | 14332 | 14333 | 357 | 14334 | 359 | 360 |
| 398.Y.T & 826.V.M | 352 | 353 | 345 | 14332 | 14302 | 357 | 14334 | 14306 | 360 |
| 398.Y.T & 887.T.D | 352 | 353 | 345 | 14332 | 14302 | 357 | 14334 | 14304 | 360 |
| 398.Y.T & 891.S.Q | 352 | 353 | 345 | 14332 | 14302 | 357 | 14334 | 14307 | 360 |
| 27.—.R & 35.R.P | 14315 | 353 | 345 | 14332 | 14333 | 357 | 14334 | 359 | 360 |
| 9.K.G & 27.—.R | 14316 | 353 | 345 | 14332 | 14333 | 357 | 14334 | 359 | 360 |
| 5.—.G & 9.K.G | 14317 | 353 | 345 | 14332 | 14333 | 357 | 14334 | 359 | 360 |
| 4.I.G & 5.—.G | 14318 | 353 | 345 | 14332 | 14333 | 357 | 14334 | 359 | 360 |
| 826.V.M & 887.T.D | 352 | 353 | 345 | 14332 | 14333 | 357 | 14334 | 14330 | 360 |
| 826.V.M & 891.S.Q | 352 | 353 | 345 | 14332 | 14333 | 357 | 14334 | 14331 | 360 |
| 5.K.G & 27.—.R | 14319 | 353 | 345 | 14332 | 14333 | 357 | 14334 | 359 | 360 |
| 5.K.G & 169.L.K | 14292 | 353 | 14297 | 14332 | 14333 | 357 | 14334 | 359 | 360 |
| 5.K.G & 171.A.D | 14292 | 353 | 14289 | 14332 | 14333 | 357 | 14334 | 359 | 360 |
| 5.K.G & 304.M.T | 14292 | 353 | 345 | 14300 | 14333 | 357 | 14334 | 359 | 360 |
| 5.K.G & 398.Y.T | 14292 | 353 | 345 | 14332 | 14302 | 357 | 14334 | 359 | 360 |
| 5.K.G & 891.S.Q | 14292 | 353 | 345 | 14332 | 14333 | 357 | 14334 | 14307 | 360 |
| 6.—.G & 27.—.R | 14320 | 353 | 345 | 14332 | 14333 | 357 | 14334 | 359 | 360 |
| 6.—.G & 169.L.K | 14293 | 353 | 14297 | 14332 | 14333 | 357 | 14334 | 359 | 360 |
| 6.—.G & 171.A.D | 14293 | 353 | 14289 | 14332 | 14333 | 357 | 14334 | 359 | 360 |
| 6.—.G & 304.M.T | 14293 | 353 | 345 | 14300 | 14333 | 357 | 14334 | 359 | 360 |
| 6.—.G & 398.Y.T | 14293 | 353 | 345 | 14332 | 14302 | 357 | 14334 | 359 | 360 |
| 6.—.G & 891.S.Q | 14293 | 353 | 345 | 14332 | 14333 | 357 | 14334 | 14307 | 360 |
| 304.M.W & 27.—.R | 14287 | 353 | 345 | 14301 | 14333 | 357 | 14334 | 359 | 360 |
| 304.M.W & 169.L.K | 352 | 353 | 14297 | 14301 | 14333 | 357 | 14334 | 359 | 360 |
| 304.M.W & 171.A.D | 352 | 353 | 14289 | 14301 | 14333 | 357 | 14334 | 359 | 360 |
| 304.M.W & 398.Y.T | 352 | 353 | 345 | 14301 | 14302 | 357 | 14334 | 359 | 360 |
| 304.M.W & 891.S.Q | 352 | 353 | 345 | 14301 | 14333 | 357 | 14334 | 14307 | 360 |
| 481.E.D & 27.—.R | 14287 | 353 | 345 | 14332 | 14303 | 357 | 14334 | 359 | 360 |
| 481.E.D & 169.L.K | 352 | 353 | 14297 | 14332 | 14303 | 357 | 14334 | 359 | 360 |
| 481.E.D & 171.A.D | 352 | 353 | 14289 | 14332 | 14303 | 357 | 14334 | 359 | 360 |
| 481.E.D & 304.M.T | 352 | 353 | 345 | 14300 | 14303 | 357 | 14334 | 359 | 360 |
| 481.E.D & 398.Y.T | 352 | 353 | 345 | 14332 | 14328 | 357 | 14334 | 359 | 360 |
| 481.E.D & 891.S.Q | 352 | 353 | 345 | 14332 | 14303 | 357 | 14334 | 14307 | 360 |
| 698.S.R & 27.—.R | 14287 | 353 | 345 | 14332 | 14333 | 357 | 14308 | 359 | 360 |
| 698.S.R & 169.L.K | 352 | 353 | 14297 | 14332 | 14333 | 357 | 14308 | 359 | 360 |
| 698.S.R & 171.A.D | 352 | 353 | 14289 | 14332 | 14333 | 357 | 14308 | 359 | 360 |
| 698.S.R & 304.M.T | 352 | 353 | 345 | 14300 | 14333 | 357 | 14308 | 359 | 360 |
| 698.S.R & 398.Y.T | 352 | 353 | 345 | 14332 | 14302 | 357 | 14308 | 359 | 360 |
| 698.S.R & 891.S.Q | 352 | 353 | 345 | 14332 | 14333 | 357 | 14308 | 14307 | 360 |

*Positions of mutations are shown relative to a CasX 515 sequence with an N-terminal methionine residue (i.e., SEQ ID NO: 5, with the addition of an N-terminal methionine).

A subset of these 161 engineered CasX proteins were cloned using methods standard in the art, and are listed in Tables 56, 58, and 59, below. In addition, an engineered CasX protein termed CasX 1001 was generated by combining mutations from engineered CasX protein 812 and CasX variant 676 (27 . . . . R, 169.L.K, and 329.G.K mutations relative to CasX 515), which have been previously validated as a highly specific and highly active CasX proteins, respectively (the PAM-altering 224.G.S mutation also present in CasX 676 was not included). Engineered CasX protein 969 was generated by combining 27 . . . . R, 171.A.D, and 224.G.T mutations relative to CasX 515. Finally, engineered CasX protein 973 was generated by combining 35.R.P, 171.A.Y, and 304.M.T mutations relative to CasX 515. The amino acid sequences of engineered CasX proteins 969, 973, and 1001 are provided in Table 55, below. In some embodiments, an engineered CasX protein of SEQ ID NO: 14352, 14353, or 14354 has improved characteristics compared to the unmodified parental CasX 515. In some embodiments, the improved characteristic is determined in an in vitro assay under comparable conditions.

TABLE 55
Amino acid sequences of engineered CasX proteins 969, 973, and 1001

| CasX | | SEQ |
|---|---|---|
| protein | | ID |
| no. | Amino acid sequence | NO |
| 969 | QEIKRINKIRRRLVKDSNTKKAGKTRGPMKTLLVRVMTPDLRERLENLRKKPENIPQPISNT | 14352 |
| | SRANLNKLLTDYTEMKKAILHVYWEEFQKDPVGLMSRVAQPASKKIDQNKLKPEMDEKGNLT | |
| | TAGFACSQCGQPLFVYKLEQVSEKGKAYTNYFGRCNVAEHEKLILLDQLKPEKDSDEAVTYS | |
| | LGKFGQRALDFYSIHVTKESTHPVKPLAQIAGNRYASTPVGKALSDACMGTIASFLSKYQDI | |
| | IIEHQKVVKGNQKRLESLRELAGKENLEYPSVTLPPQPHTKEGVDAYNEVIARVRMWVNLNL | |
| | WQKLKLSRDDAKPLLRLKGFPSFPLVERQANEVDWWDMVCNVKKLINEKKEDGKVFWQNLAG | |
| | YKRQEALRPYLSSEEDRKKGKKFARYQLGDLLLHLEKKHGEDWGKVYDEAWERIDKKVEGLS | |
| | KHIKLEEERRSEDAQSKAALTDWLRAKASFVIEGLKEADKDEFCRCELKLQKWYGDLRGKPF | |
| | AIEAENSILDISGFSKQYNCAFIWQKDGVKKLNLYLIINYFKGGKLRFKKIKPEAFEANRFY | |
| | TVINKKSGEIVPMEVNENFDDPNLIILPLAFGKRQGREFIWNDLLSLETGSLKLANGRVIEK | |
| | TLYNRRTRQDEPALFVALTFERREVLDSSNIKPMNLIGVDRGENIPAVIALTDPEGCPLSRF | |
| | KDSLGNPTHILRIGESYKEKQRTIQAKKEVEQRRAGGYSRKYASKAKNLADDMVRNTARDLL | |
| | YYAVTQDAMLIFENLSRGFGRQGKRTEMAERQYTRMEDWLTAKLAYEGLPSKTYLSKTLAQY | |
| | TSKTCSNCGFTITSADYDRVLEKLKKTATGWMTTINGKELKVEGQITYYNRYKRQNVVKDLS | |
| | VELDRLSEESVNNDISSWTKGRSGEALSLLKKRFSHRPVQEKFVCLNCGFETHADEQAALNI | |
| | ARSWLFLRSQEYKKYQTNKTTGNTDKRAFVETWQSFYRKKLKEVWKPAV | |
| 973 | QEIKRINKIRRRLVKDSNTKKAGKTGPMKTLLVPVMTPDLRERLENLRKKPENIPQPISNTS | 14353 |
| | RANLNKLLTDYTEMKKAILHVYWEEFQKDPVGLMSRVAQPASKKIDQNKLKPEMDEKGNLTT | |
| | AGFACSQCGQPLFVYKLEQVSEKGKAYTNYFGRCNVAEHEKLILLYQLKPEKDSDEAVTYSL | |
| | GKFGQRALDFYSIHVTKESTHPVKPLAQIAGNRYASGPVGKALSDACMGTIASFLSKYQDII | |
| | IEHQKVVKGNQKRLESLRELAGKENLEYPSVTLPPQPHTKEGVDAYNEVIARVRTWVNLNLW | |
| | QKLKLSRDDAKPLLRLKGFPSFPLVERQANEVDWWDMVCNVKKLINEKKEDGKVFWQNLAGY | |
| | KRQEALRPYLSSEEDRKKGKKFARYQLGDLLLHLEKKHGEDWGKVYDEAWERIDKKVEGLSK | |
| | HIKLEEERRSEDAQSKAALTDWLRAKASFVIEGLKEADKDEFCRCELKLQKWYGDLRGKPFA | |
| | IEAENSILDISGFSKQYNCAFIWQKDGVKKLNLYLIINYFKGGKLRFKKIKPEAFEANRFYT | |
| | VINKKSGEIVPMEVNENFDDPNLIILPLAFGKRQGREFIWNDLLSLETGSLKLANGRVIEKT | |
| | LYNRRTRQDEPALFVALTFERREVLDSSNIKPMNLIGVDRGENIPAVIALTDPEGCPLSRFK | |
| | DSLGNPTHILRIGESYKEKQRTIQAKKEVEQRRAGGYSRKYASKAKNLADDMVRNTARDLLY | |
| | YAVTQDAMLIFENLSRGFGRQGKRTFMAERQYTRMEDWLTAKLAYEGLPSKTYLSKTLAQYT | |
| | SKTCSNCGFTITSADYDRVLEKLKKTATGWMTTINGKELKVEGQITYYNRYKRQNVVKDLSV | |
| | ELDRLSEESVNNDISSWTKGRSGEALSLLKKRFSHRPVQEKFVCLNCGFETHADEQAALNIA | |
| | RSWLFLRSQEYKKYQTNKTTGNTDKRAFVETWQSFYRKKLKEVWKPAV | |
| 1001 | QEIKRINKIRRRLVKDSNTKKAGKTRGPMKTLLVRVMTPDLRERLENLRKKPENIPQPISNT | 14354 |
| | SRANLNKLLTDYTEMKKAILHVYWEEFQKDPVGLMSRVAQPASKKIDQNKLKPEMDEKGNLT | |
| | TAGFACSQCGQPLFVYKLEQVSEKGKAYTNYFGRCNVAEHEKLIKLAQLKPEKDSDEAVTYS | |
| | LGKFGQRALDFYSIHVTKESTHPVKPLAQIAGNRYASGPVGKALSDACMGTIASFLSKYQDI | |
| | IIEHQKVVKGNQKRLESLRELAGKENLEYPSVTLPPQPHTKEGVDAYNEVIARVRMWVNLNL | |
| | WQKLKLSRDDAKPLLRLKKFPSFPLVERQANEVDWWDMVCNVKKLINEKKEDGKVFWQNLAG | |
| | YKRQEALRPYLSSEEDRKKGKKFARYQLGDLLLHLEKKHGEDWGKVYDEAWERIDKKVEGLS | |
| | KHIKLEEERRSEDAQSKAALTDWLRAKASFVIEGLKEADKDEFCRCELKLQKWYGDLRGKPF | |
| | AIEAENSILDISGFSKQYNCAFIWQKDGVKKLNLYLIINYFKGGKLRFKKIKPEAFEANRFY | |
| | TVINKKSGEIVPMEVNENFDDPNLIILPLAFGKRQGREFIWNDLLSLETGSLKLANGRVIEK | |
| | TLYNRRTRQDEPALFVALTFERREVLDSSNIKPMNLIGVDRGENIPAVIALTDPEGCPLSRF | |
| | KDSLGNPTHILRIGESYKEKQRTIQAKKEVEQRRAGGYSRKYASKAKNLADDMVRNTARDLL | |
| | YYAVTQDAMLIFENLSRGFGRQGKRTEMAERQYTRMEDWLTAKLAYEGLPSKTYLSKTLAQY | |
| | TSKTCSNCGFTITSADYDRVLEKLKKTATGWMTTINGKELKVEGQITYYNRYKRQNVVKDLS | |
| | VELDRLSEESVNNDISSWTKGRSGEALSLLKKRFSHRPVQEKFVCLNCGFETHADEQAALNI | |
| | ARSWLFLRSQEYKKYQTNKTTGNTDKRAFVETWQSFYRKKLKEVWKPAV | |

A multiplexed pooled PASS assay was performed and analyzed as described in Example 25. As noted in Example 25, CasX proteins were expressed using a relatively weakly-expressing promoter to reduce CasX protein expression and thereby improve the sensitivity of the assay. Samples were tested in duplicate, except for engineered CasX protein 1006, which was tested in quadruplicate. In Tables 56, 58, and 59, below, the results for the CasX 1006 samples are reported in two separate rows, each the average of two samples. Streptococcus pyogenes Cas9 without a guide RNA served as a negative control. CasX 515, CasX 676, and engineered CasX protein 812 were also included as controls.

### Results

Table 56 provides the level of on-target editing produced by various CasX proteins with mutations relative to CasX 515, ranked from highest to lowest activity.

TABLE 56
Average on-targeting editing activity of engineered CasX proteins, ranked from
highest to lowest

| Protein | Mutation(s) relative | | | |
|---|---|---|---|---|
| Designation | to CasX 515* | Amino acid | Average on-target | SEM on-target TTC |
| (CasX protein | (Position.Reference. | sequence | TTC PAM editing | PAM editing |
| no., or Cas9) | Alternative) | SEQ ID NO | activity (fraction) | activity (fraction) |
| 1018 | 9.K.G & 891.S.Q | 14161 | 3.01E−01 | 1.20E−01 |
| 1007 | 304.M.T & 826.V.M | 14243 | 2.76E−01 | 7.86E−02 |
| 1006 | 826.V.M & 891.S.Q | 14257 | 2.38E−01 | 4.11E−02 |
| 987 | 169.L.K & 304.M.T | 14202 | 2.32E−01 | 3.38E−02 |
| 1014 | 4.I.G & 891.S.Q | 14137 | 2.29E−01 | 3.45E−02 |
| 1143 | 4.I.G & 826.V.M | 14135 | 2.29E−01 | 3.44E−02 |
| 1019 | 27.—.R & 171.A.S | 14167 | 2.22E−01 | 2.52E−02 |
| 1029 | 5.K.G & 891.S.Q | 14263 | 2.16E−01 | 2.96E−02 |
| 1006 | 826.V.M & 891.S.Q | 14257 | 2.16E−01 | 3.44E−02 |
| 1015 | 5.—.G & 304.M.T | 14145 | 2.11E−01 | 3.20E−02 |
| 970 | 27.—.R & 891.S.Q | 14173 | 2.09E−01 | 2.61E−02 |
| 1028 | 5.K.G & 304.M.T | 14261 | 2.07E−01 | 3.10E−02 |
| 996 | 171.A.Y & 891.S.Q | 14227 | 2.05E−01 | 3.42E−02 |
| 969 | 27.—. R, 171.A.D, & | 14352 | 2.04E−01 | 2.64E−02 |
| | 224.G.T | | | |
| 984 | 64.R.Q & 891.S.Q | 14197 | 2.03E−01 | 9.13E−02 |
| 1041 | 698.S.R & 891.S.Q | 14286 | 2.01E−01 | 3.17E−02 |
| 1016 | 9.K.G & 171.A.D | 14153 | 1.98E−01 | 3.20E−02 |
| 1000 | 224.G.T & 891.S.Q | 14239 | 1.92E−01 | 2.51E−02 |
| 999 | 224.G.T & 304.M.T | 14235 | 1.92E−01 | 2.90E−02 |
| 986 | 169.L.K & 171.A.S | 14200 | 1.90E−01 | 2.76E−02 |
| 977 | 64.R.Q & 169.L.K | 14187 | 1.90E−01 | 3.12E−02 |
| 792 | 27.- R & 169.L.K | 14163 | 1.89E−01 | 2.89E−02 |
| 993 | 171.A.D & 224.G.T | 14216 | 1.88E−01 | 2.70E−02 |
| 997 | 171.A.S & 304.M.T | 14229 | 1.87E−01 | 2.83E−02 |
| 1025 | 169.L.K & 891.S.Q | 14206 | 1.87E−01 | 2.99E−02 |
| 1001 | 27.—.R, 169.L.K, & | 14354 | 1.85E−01 | 2.57E−02 |
| | 329.G.K | | | |
| 1040 | 481.E.D & 891.S.Q | 14280 | 1.85E−01 | 3.00E−02 |
| 1004 | 304.M.T & 891.S.Q | 14245 | 1.85E−01 | 3.11E−02 |
| 676 | 27.—.R, 170.L.K, & | 6 | 1.84E−01 | 2.28E−02 |
| | 224.G.S | | | |
| 1031 | 6.—.G & 169.L.K | 14265 | 1.84E−01 | 3.02E−02 |
| 980 | 64.R.Q & 171.A.S | 14191 | 1.80E−01 | 3.06E−02 |
| 981 | 64.R.Q & 304.M.T | 14193 | 1.79E−01 | 2.44E−02 |
| 985 | 169.L.K & 171.A.Y | 14199 | 1.73E−01 | 3.57E−02 |
| 989 | 169.L.Q & 224.G.T | 14210 | 1.73E−01 | 3.20E−02 |
| 992 | 169.L.Q & 887.T.D | 14214 | 1.70E−01 | 3.40E−02 |
| 99 | 171.A.Y & 224.G.T | 14222 | 1.68E−01 | 3.21E−02 |
| 1005 | 826.V.M & 887.T.D | 14256 | 1.68E−01 | 3.17E−02 |
| 983 | 64.R.Q & 887.T.D | 14196 | 1.68E−01 | 1.70E−02 |
| 1026 | 169.L.Q & 826.V.M | 14213 | 1.65E−01 | 3.09E−02 |
| 1009 | 4.I.G & 171.A.D | 14129 | 1.65E−01 | 6.43E−02 |
| 982 | 64.R.Q & 398.Y.T | 14194 | 1.64E−01 | 2.73E−02 |
| 978 | 64.R.Q & 169.L.Q | 14188 | 1.63E−01 | 2.96E−02 |
| 515 | — | 5 | 1.63E−01 | 2.38E−02 |
| 1003 | 9.K.G & 27.—.R | 14253 | 1.61E−01 | 2.28E−02 |
| 1017 | 9.K.G & 224.G.T | 14156 | 1.56E−01 | 2.73E−02 |
| 1020 | 35.R.P & 171.A.D | 14177 | 1.55E−01 | 3.01E−02 |
| 998 | 171.A.S & 826.V.M | 14231 | 1.54E−01 | 2.92E−02 |
| 1010 | 4.I.G & 171.A.Y | 14130 | 1.54E−01 | 2.71E−02 |
| 1022 | 35.R.P & 891.S.Q | 14185 | 1.48E−01 | 2.79E−02 |
| 1038 | 224.G.T & 826.V.M | 14237 | 1.48E−01 | 3.11E−02 |
| 1027 | 5.K.G & 171.A.D | 14260 | 1.42E−01 | 2.32E−02 |
| 1012 | 4.I.G & 398.Y.T | 14134 | 1.40E−01 | 2.10E−02 |
| 971 | 35.R.P & 169.L.Q | 14176 | 1.39E−01 | 2.75E−02 |
| 1032 | 6.—.G & 171.A.D | 14266 | 1.39E−01 | 2.40E−02 |
| 1024 | 169.L.K & 398.Y.T | 14203 | 1.38E−01 | 2.87E−02 |
| 1023 | 64.R.Q & 224.G.T | 14192 | 1.37E−01 | 2.34E−02 |
| 1036 | 171.A.S & 887.T.D | 14232 | 1.37E−01 | 2.38E−02 |
| 988 | 169.L.Q & 171.A.Y | 14208 | 1.36E−01 | 3.36E−02 |
| 1034 | 171.A.Y & 826.V.M | 14225 | 1.36E−01 | 3.05E−02 |
| 1030 | 171.A.D & 398.Y.T | 14218 | 1.35E−01 | 2.55E−02 |
| 1039 | 304.M.W & 398.Y.T | 14273 | 1.29E−01 | 3.28E−02 |
| 1033 | 171.A.Y & 304.M.T | 14223 | 1.25E−01 | 2.29E−02 |
| 1021 | 35.R.P & 304.M.T | 14181 | 1.25E−01 | 1.77E−02 |
| 1011 | 4.I.G & 224.G.T | 14132 | 1.24E−01 | 2.13E−02 |
| 979 | 64.R.Q & 171.A.Y | 14190 | 1.24E−01 | 2.15E−02 |
| 1002 | 5.—.G & 35.R.P | 14240 | 1.20E−01 | 2.31E−02 |
| 1035 | 171.A.S & 398.Y.T | 14230 | 1.14E−01 | 2.07E−02 |
| 851 | 35.R.P & 171.A.Y | 14178 | 1.13E−01 | 2.31E−02 |
| 995 | 171.A.Y & 398.Y.T | 14224 | 1.13E−01 | 1.83E−02 |
| 973 | 35.R.P, 171.A.Y, & | 14353 | 1.12E−01 | 1.96E−02 |
| | 304.M.T | | | |
| 976 | 35.R.P & 887.T.D | 14184 | 9.62E−02 | 2.52E−02 |
| 974 | 35.R.P & 224.G.T | 14180 | 9.00E−02 | 1.52E−02 |
| 1037 | 224.G.T & 398.Y.T | 14236 | 8.30E−02 | 1.35E−02 |
| 812 | 329.G.K | 7 | 7.68E−02 | 1.69E−02 |
| 975 | 35.R.P & 398.Y.T | 14182 | 7.49E−02 | 1.51E−02 |
| 991 | 169.L.Q & 398.Y.T | 14212 | 4.87E−02 | 1.74E−02 |
| Cas9 | n/a | — | 0.00E+00 | 0.00E+00 |

*Positions of mψtations are shown relative to a CasX 515 sequence with an N-terminal methionine residue (i.e., SEQ ID NO: 5, with the addition of an N-terminal methionine).

As shown in Table 56, 41 of the tested engineered CasX proteins produced higher levels of on-target editing than did CasX 515; the 41 CasX proteins are bolded in Table 56. Engineered CasX protein 1018 had 9.K.G and 891.S.Q amino acid substitutions and produced the highest level of on-target editing in the assay. The CasX 676 control was more active than CasX 515, and CasX 812 was less active than CasX 515, which is consistent with previous results.

A large number of the tested CasX proteins produced lower levels of on-target editing than CasX 515. This suggests that not all combinations of mutations, including combinations of mutations that were relatively active for on-target editing when introduced into CasX 515 as single mutations (see Example 25), are compatible for producing highly active CasX proteins.

To understand the amino acid residues that may be causal for improving CasX activity, the identity of the mutations in the engineered CasX proteins with two or three mutations resulting in improved on-target editing activity relative to CasX 515 was examined (Table 57).

TABLE 57
Summary of mutations in engineered CasX proteins with
greater on-target editing activity than CasX 515

| Position of mutation | | |
|---|---|---|
| relative to CasX 515 | | |
| (SEQ ID NO: 5, | Number of | Identity of |
| with the addition | occurrences | mutation(s) (Number |
| of an N-terminal | of mutations | of engineered CasX |
| methionine) | at position* | proteins with mutation) |
| 891 | 13 | 891.S.Q |
| 169 | 12 | 169.L.K (8); 169.L.Q (4) |
| 171 | 11 | 171.A.S (4); 171.A.D (4); |
| | | 171.A.Y (3) |
| 304 | 8 | 304.M.T |
| 64 | 7 | 64.R.Q |
| 224 | 6 | 224.G.T |
| 27 | 5 | 27.—.R |
| 826 | 4 | 826.V.M |
| 4 | 3 | 4.I.G |
| 5 | 3 | 5.—.G (1); 5.K.G (2) |
| 887 | 3 | 887.T.D |
| 9 | 2 | 9.K.G |
| 6 | 1 | 6.—.G |
| 481 | 1 | 481.E.D |
| 698 | 1 | 698.S.R |

*Excluding CasX 676.

As shown in Table 57, certain positions were mutated in several members of the set of engineered CasX proteins with higher on-target editing activity than CasX 515. For example, the serine to glutamine substitution at position 891 (891.S.Q), in the TSL domain, was found in 13 members of the engineered CasX proteins with improved on-target editing activity relative to CasX 515. The TSL domain is a dynamic domain involved in coordinating the introduction of the target strand to the RuvC active site, and the substitution of serine for the longer glutamine may allow for additional hydrogen bonding interactions with the target strand and more efficient transfer to the nuclease domain.

One of two substitutions at position 169 (169.L.K or 169.L.Q), in the NTSB domain, were found in 12 members of the engineered CasX proteins with higher on-target editing activity than CasX 515. This position is proximal to the second and third nucleotides of the unwound non-target strand in structures of the non-target strand loading state, and the introduction of either a charged residue or one capable of multiple hydrogen-bonding interactions likely allows for the stabilization of the unwound state and thus more efficient unwinding. It should be noted that 169.L.K was more enriched than 169.L.Q among the engineered CasX proteins with improved on-target editing activity, which suggests that while a polar interaction increases enzymatic activity, a charge-charge interaction is more suitable for this position.

One of three substitutions at position 171 (171.A.S, 171.A.D, or 171.A.Y), also in the NTSB domain, were found in 11 members of the engineered CasX proteins with improved on-target editing activity. Residue 171 is solvent-exposed, so a polar residue is likely more favorable at this position. While the residue is not in a position that interacts with the non-target strand in published structures, the dynamic nature of the NTSB domain may allow these residues to make hydrogen-bonding interactions with the target DNA at some point in the unwinding process. A serine is present at this position in the wild-type CasX 2 (SEQ ID NO: 2) sequence and is an alanine in CasX variants containing the chimeric NTSB from CasX1, meaning that the 171.A.S mutation in particular represents a reversion to a wild-type sequence. Notably, 171.A. Y was also found in several of the variants performing worse than CasX 515, which suggests that a tyrosine at position 171 might create too much steric hindrance for proper hydrogen-bonding interactions with the target DNA.

While the 169.L.K and 27.-.R mutations found in CasX 676 were well-represented among the high activity variants, there were a number of orthogonal mutations with distinct mechanisms that may allow for increased activity without the loss of specificity seen in CasX 676. 891.S.Q in particular was found in a number of top-performing activity variants that also have a higher specificity ratio than CasX 515 (see below).

Table 58, below, provides the level of off-target editing produced by various CasX proteins with two or three mutations relative to CasX 515, ranked from lowest (meaning the highest specificity) to highest activity.

TABLE 58
Average off-targeting editing activity of engineered
CasX proteins, ranked from lowest to highest

| Protein | | | Average | SEM |
|---|---|---|---|---|
| Designation | | Amino | off-target | off-target |
| (CasX | Mutation relative | acid | TTC PAM | TTC PAM |
| protein | to CasX 515* | sequence | editing | editing |
| no., or | (Position.Refer- | SEQ | activity | activity |
| Cas9) | ence.Alternative) | ID NO | (fraction) | (fraction) |
| Cas9 | n/a | — | 0.00E+00 | 0.00E+00 |
| 812 | 329.G.K | 7 | 4.46E−03 | 3.30E−03 |
| 991 | 169.L.Q & 398.Y.T | 14212 | 4.71E−03 | 3.59E−03 |
| 975 | 35.R.P & 398.Y.T | 14182 | 6.63E−03 | 5.75E−03 |
| 976 | 35.R.P & 887.T.D | 14184 | 8.76E−03 | 6.93E−03 |
| 851 | 35.R.P & 171.A.Y | 14178 | 9.95E−03 | 6.91E−03 |
| 974 | 35.R.P & 224.G.T | 14180 | 9.95E−03 | 6.79E−03 |
| 971 | 35.R.P & 169.L.Q | 14176 | 1.15E−02 | 8.07E−03 |
| 1037 | 224.G.T & 398.Y.T | 14236 | 1.22E−02 | 8.00E−03 |
| 1039 | 304.M.W & 398.Y.T | 14273 | 1.24E−02 | 1.01E−02 |
| 988 | 169.L.Q & 171.A.Y | 14208 | 1.30E−02 | 8.38E−03 |
| 973 | 35.R.P, 171.A.Y, & | 14353 | 1.37E−02 | 9.35E−03 |
| | 304.M.T | | | |
| 995 | 171.A.Y & 398.Y.T | 14224 | 1.53E−02 | 9.56E−03 |
| 1002 | 5.—.G & 35.R.P | 14240 | 1.69E−02 | 1.03E−02 |
| 1009 | 4.I.G & 171.A.D | 14129 | 1.76E−02 | 1.08E−02 |
| 1011 | 4.I.G & 224.G.T | 14132 | 1.80E−02 | 1.12E−02 |
| 1035 | 171.A.S & 398.Y.T | 14230 | 1.86E−02 | 9.86E−03 |
| 989 | 169.L.Q & 224.G.T | 14210 | 1.91E−02 | 1.19E−02 |
| 1018 | 9.K.G & 891.S.Q | 14161 | 1.91E−02 | 1.43E−02 |
| 1033 | 171.A.Y & 304.M.T | 14223 | 1.93E−02 | 1.09E−02 |
| 994 | 171.A.Y & 224.G.T | 14222 | 1.95E−02 | 1.21E−02 |
| 1020 | 35.R.P & 171.A.D | 14177 | 1.98E−02 | 1.24E−02 |
| 1022 | 35.R.P & 891.S.Q | 14185 | 2.04E−02 | 1.10E−02 |
| 979 | 64.R.Q & 171.A.Y | 14190 | 2.09E−02 | 1.24E−02 |
| 1032 | 6.—.G & 171.A.D | 14266 | 2.20E−02 | 1.15E−02 |
| 1027 | 5.K.G & 171.A.D | 14260 | 2.28E−02 | 1.29E−02 |
| 1041 | 698.S.R & 891.S.Q | 14286 | 2.41E−02 | 1.32E−02 |
| 1012 | 4.I.G & 398.Y.T | 14134 | 2.50E−02 | 1.30E−02 |
| 1030 | 171.A.D & 398.Y.T | 14218 | 2.53E−02 | 1.31E−02 |
| 1024 | 169.L.K & 398.Y.T | 14203 | 2.54E−02 | 1.39E−02 |
| 982 | 64.R.Q & 398.Y.T | 14194 | 2.58E−02 | 1.58E−02 |
| 983 | 64.R.Q & 887.T.D | 14196 | 2.59E−02 | 1.06E−02 |
| 1040 | 481.E.D & 891.S.Q | 14280 | 2.63E−02 | 1.30E−02 |
| 1017 | 9.K.G & 224.G.T | 14156 | 2.67E−02 | 1.64E−02 |
| 1001 | 27.—.R, 169.L.K, | 14354 | 2.74E−02 | 1.31E−02 |
| | & 329.G.K | | | |
| 992 | 169.L.Q & 887.T.D | 14214 | 2.78E−02 | 1.47E−02 |
| 1036 | 171.A.S & 887.T.D | 14232 | 2.80E−02 | 1.47E−02 |
| 1003 | 9.K.G & 27.—.R | 14253 | 2.80E−02 | 1.40E−02 |
| 1005 | 826.V.M & 887.T.D | 14256 | 2.81E−02 | 1.41E−02 |
| 978 | 64.R.Q & 169.L.Q | 14188 | 2.83E−02 | 1.48E−02 |
| 1021 | 35.R.P & 304.M.T | 14181 | 2.84E−02 | 1.62E−02 |
| 985 | 169.L.K & 171.A.Y | 14199 | 3.00E−02 | 1.62E−02 |
| 977 | 64.R.Q & 169.L.K | 14187 | 3.03E−02 | 1.46E−02 |
| 1023 | 64.R.Q & 224.G.T | 14192 | 3.05E−02 | 1.81E−02 |
| 1034 | 171.A.Y & 826.V.M | 14225 | 3.07E−02 | 1.24E−02 |
| 1010 | 4.I.G & 171.A.Y | 14130 | 3.17E−02 | 1.59E−02 |
| 1026 | 169.L.Q & 826.V.M | 14213 | 3.18E−02 | 1.44E−02 |
| 1016 | 9.K.G & 171.A.D | 14153 | 3.30E−02 | 1.43E−02 |
| 1038 | 224.G.T & 826.V.M | 14237 | 3.30E−02 | 1.58E−02 |
| 1029 | 5.K.G & 891.S.Q | 14263 | 3.31E−02 | 1.77E−02 |
| 1031 | 6.—.G & 169.L.K | 14265 | 3.38E−02 | 1.65E−02 |
| 980 | 64.R.Q & 171.A.S | 14191 | 3.41E−02 | 1.59E−02 |
| 993 | 171.A.D & 224.G.T | 14216 | 3.43E−02 | 1.68E−02 |
| 1028 | 5.K.G & 304.M.T | 14261 | 3.65E−02 | 1.85E−02 |
| 998 | 171.A.S & 826.V.M | 14231 | 3.68E−02 | 1.63E−02 |
| 515 | — | 5 | 3.83E−02 | 1.46E−02 |
| 999 | 224.G.T & 304.M.T | 14235 | 3.91E−02 | 1.93E−02 |
| 1000 | 224.G.T & 891.S.Q | 14239 | 4.14E−02 | 2.07E−02 |
| 996 | 171.A.Y & 891.S.Q | 14227 | 4.25E−02 | 2.02E−02 |
| 981 | 64.R.Q & 304.M.T | 14193 | 4.30E−02 | 2.20E−02 |
| 1014 | 4.I.G & 891.S.Q | 14137 | 4.63E−02 | 2.06E−02 |
| 986 | 169.L.K & 171.A.S | 14200 | 4.63E−02 | 2.46E−02 |
| 1006 | 826.V.M & 891.S.Q | 14257 | 4.66E−02 | 2.16E−02 |
| 1025 | 169.L.K & 891.S.Q | 14206 | 4.73E−02 | 2.04E−02 |
| 997 | 171.A.S & 304.M.T | 14229 | 4.82E−02 | 2.22E−02 |
| 1015 | 5.—.G & 304.M.T | 14145 | 5.10E−02 | 2.19E−02 |
| 987 | 169.L.K & 304.M.T | 14202 | 5.72E−02 | 2.55E−02 |
| 1006 | 826.V.M & 891.S.Q | 14257 | 5.87E−02 | 2.71E−02 |
| 969 | 27.—.R, 171.A.D, | 14352 | 5.88E−02 | 2.17E−02 |
| | & 224.G.T | | | |
| 1004 | 304.M.T & 891.S.Q | 14245 | 6.11E−02 | 2.26E−02 |
| 1143 | 4.I.G & 826.V.M | 14135 | 6.15E−02 | 2.79E−02 |
| 676 | 27.—.R, 170.L.K, | 6 | 6.19E−02 | 2.14E−02 |
| | & 224.G.S | | | |
| 1019 | 27.—.R & 171.A.S | 14167 | 6.78E−02 | 1.45E−02 |
| 970 | 27.—.R & 891.S.Q | 14173 | 6.87E−02 | 2.22E−02 |
| 1007 | 304.M.T & 826.V.M | 14243 | 6.99E−02 | 8.52E−02 |
| 984 | 64.R.Q & 891.S.Q | 14197 | 7.16E−02 | 1.01E−01 |
| 792 | 27.—.R & 169.L.K | 14163 | 7.26E−02 | 2.39E−02 |

As shown in Table 58, the majority of the tested CasX proteins with pairs of mutations relative to CasX 515 produced lower levels of off-target editing than did CasX 515; these samples are bolded in Table 58.

Table 59, below, provides the specificity ratio (i.e., the average level of on-targeting editing divided by the average level of off-target editing) of the tested CasX proteins with two or three mutations relative to CasX 515, ranked from the highest to lowest ratio. CasX proteins with higher specificity ratios than CasX 515 are bolded in Table 59.

TABLE 59
Specificity ratios of engineered CasX proteins,
ranked from highest to lowest*

| | | | Specificity | |
|---|---|---|---|---|
| | | | ratio | |
| Protein | | | (average | SEM |
| Designation | | Amino | on-target | off-target |
| (CasX | Mutation relative | acid | activity/ | TTC PAM |
| protein | to CasX 515* | sequence | average | editing |
| no., or | (Position.Refer- | (SEQ | off-target | activity |
| Cas9) | ence.Alternative) | ID NO) | activity) | (fraction) |
| 812 | 329.G.K | 7 | 17.22 | 0.52 |
| 1018 | 9.K.G & 891.S.Q | 14161 | 15.76 | 0.35 |
| 971 | 35.R.P & 169.L.Q | 14176 | 12.09 | 0.5 |
| 851 | 35.R.P & 171.A.Y | 14178 | 11.36 | 0.49 |
| 975 | 35.R.P & 398.Y.T | 14182 | 11.3 | 0.67 |
| 976 | 35.R.P & 887.T.D | 14184 | 10.98 | 0.53 |
| 988 | 169.L.Q & 171.A.Y | 14208 | 10.46 | 0.4 |
| 1039 | 304.M.W & 398.Y.T | 14273 | 10.4 | 0.56 |
| 991 | 169.L.Q & 398.Y.T | 14212 | 10.34 | 0.41 |
| 1009 | 4.I.G & 171.A.D | 14129 | 9.38 | 0.23 |
| 989 | 169.L.Q & 224.G.T | 14210 | 9.06 | 0.44 |
| 974 | 35.R.P & 224.G.T | 14180 | 9.05 | 0.51 |
| 994 | 171.A.Y & 224.G.T | 14222 | 8.62 | 0.43 |
| 1041 | 698.S.R & 891.S.Q | 14286 | 8.34 | 0.39 |
| 973 | 35.R.P, 171.A.Y, & | 14353 | 8.18 | 0.51 |
| | 304.M.T | | | |
| 1020 | 35.R.P & 171.A.D | 14177 | 7.83 | 0.43 |
| 995 | 171.A.Y & 398.Y.T | 14224 | 7.39 | 0.46 |
| 1022 | 35.R.P & 891.S.Q | 14185 | 7.25 | 0.35 |
| 1002 | 5.—.G & 35.R.P | 14240 | 7.1 | 0.41 |
| 1040 | 481.E.D & 891.S.Q | 14280 | 7.03 | 0.33 |
| 1011 | 4.I.G & 224.G.T | 14132 | 6.89 | 0.45 |
| 1037 | 224.G.T & 398.Y.T | 14236 | 6.8 | 0.49 |
| 1001 | 27.—.R, 169.L.K, | 14354 | 6.75 | 0.34 |
| | & 329.G.K | | | |
| 1029 | 5.K.G & 891.S.Q | 14263 | 6.53 | 0.4 |
| 983 | 64.R.Q & 887.T.D | 14196 | 6.49 | 0.38 |
| 1033 | 171.A.Y & 304.M.T | 14223 | 6.48 | 0.31 |
| 982 | 64.R.Q & 398.Y.T | 14194 | 6.36 | 0.45 |
| 1032 | 6.—.G & 171.A.D | 14266 | 6.32 | 0.35 |
| 977 | 64.R.Q & 169.L.K | 14187 | 6.27 | 0.32 |
| 1027 | 5.K.G & 171.A.D | 14260 | 6.23 | 0.4 |
| 1035 | 171.A.S & 398.Y.T | 14230 | 6.13 | 0.35 |
| 992 | 169.L.Q & 887.T.D | 14214 | 6.12 | 0.33 |
| 1016 | 9.K.G & 171.A.D | 14153 | 6 | 0.27 |
| 1005 | 826.V.M & 887.T.D | 14256 | 5.98 | 0.31 |
| 979 | 64.R.Q & 171.A.Y | 14190 | 5.93 | 0.42 |
| 1017 | 9.K.G & 224.G.T | 14156 | 5.84 | 0.44 |
| 985 | 169.L.K & 171.A.Y | 14199 | 5.77 | 0.34 |
| 978 | 64.R.Q & 169.L.Q | 14188 | 5.76 | 0.33 |
| 1003 | 9.K.G & 27.—.R | 14253 | 5.75 | 0.36 |
| 1028 | 5.K.G & 304.M.T | 14261 | 5.67 | 0.36 |
| 1012 | 4.I.G & 398.Y.T | 14134 | 5.6 | 0.37 |
| 993 | 171.A.D & 224.G.T | 14216 | 5.48 | 0.34 |
| 1031 | 6.—.G & 169.L.K | 14265 | 5.44 | 0.34 |
| 1024 | 169.L.K & 398.Y.T | 14203 | 5.43 | 0.32 |
| 1030 | 171.A.D & 398.Y.T | 14218 | 5.34 | 0.33 |
| 980 | 64.R.Q & 171.A.S | 14191 | 5.28 | 0.3 |
| 1026 | 169.L.Q & 826.V.M | 14213 | 5.19 | 0.27 |
| 1006 | 826.V.M & 891.S.Q | 14257 | 5.11 | 0.29 |
| 1014 | 4.I.G & 891.S.Q | 14137 | 4.95 | 0.29 |
| 999 | 224.G.T & 304.M.T | 14235 | 4.91 | 0.34 |
| 1036 | 171.A.S & 887.T.D | 14232 | 4.89 | 0.35 |
| 1010 | 4.I.G & 171.A.Y | 14130 | 4.86 | 0.33 |
| 996 | 171.A.Y & 891.S.Q | 14227 | 4.82 | 0.31 |
| 1000 | 224.G.T & 891.S.Q | 14239 | 4.64 | 0.37 |
| 1023 | 64.R.Q & 224.G.T | 14192 | 4.49 | 0.42 |
| 1038 | 224.G.T & 826.V.M | 14237 | 4.48 | 0.27 |
| 1034 | 171.A.Y & 826.V.M | 14225 | 4.43 | 0.43 |
| 1021 | 35.R.P & 304.M.T | 14181 | 4.4 | 0.18 |
| 515 | — | 5 | 4.26 | 0.24 |
| 998 | 171.A.S & 826.V.M | 14231 | 4.18 | 0.25 |
| 981 | 64.R.Q & 304.M.T | 14193 | 4.16 | 0.37 |
| 1015 | 5.—.G & 304.M.T | 14145 | 4.14 | 0.28 |
| 986 | 169.L.K & 171.A.S | 14200 | 4.1 | 0.39 |
| 987 | 169.L.K & 304.M.T | 14202 | 4.06 | 0.3 |
| 1025 | 169.L.K & 891.S.Q | 14206 | 3.95 | 0.27 |
| 1007 | 304.M.T & 826.V.M | 14243 | 3.95 | 0.93 |
| 997 | 171.A.S & 304.M.T | 14229 | 3.88 | 0.31 |
| 1143 | 4.I.G & 826.V.M | 14135 | 3.72 | 0.3 |
| 1006 | 826.V.M & 891.S.Q | 14257 | 3.68 | 0.3 |
| 969 | 27.—.R, 171.A.D, | 14352 | 3.47 | 0.24 |
| | & 224.G.T | | | |
| 1019 | 27.—.R & 171.A.S | 14167 | 3.27 | 0.1 |
| 970 | 27.—.R & 891.S.Q | 14173 | 3.04 | 0.2 |
| 1004 | 304.M.T & 891.S.Q | 14245 | 3.03 | 0.2 |
| 676 | 27.—.R, 170.L.K, | 6 | 2.97 | 0.22 |
| | & 224.G.S | | | |
| 984 | 64.R.Q & 891.S.Q | 14197 | 2.84 | 0.95 |
| 792 | 27.—.R & 169.L.K | 14163 | 2.6 | 0.18 |

*Specificity ratio and SEM values are shown rounded to the nearest hundredth.

As shown in Table 59, the majority of the tested engineered CasX proteins had higher on-target to off-target editing ratios than CasX 515. While the previously validated high-specificity variant CasX 812 had the highest specificity ratio, many engineered CasX proteins demonstrated high specificity ratios without as significant a loss in on-target activity as was observed for CasX 812.

The 35.R.P mutation was commonly observed in variants with very high specificity ratios. This residue is in the OBD and believed to be involved in binding the guide RNA. Mutation to a proline at this position may have complex effects on allosteric regulation. Notably, these variants also tended to have low activity, suggesting that apparent specificity may be in part the result of less efficient RNP formation due to the disruption of this guide-binding interaction. Overall, an inverse correlation was observed between specificity ratio and activity. This suggests that it is difficult to fully avoid trade-offs between activity and specificity. However, it is also evident that the strategy of combining activity and specificity mutants can compensate for this trade-off and result in variants with both characteristics improved.

Notably, some engineered CasX variants produced both higher levels of on-target editing and lower levels of off-target editing than did CasX 515, namely engineered CasX proteins 977, 978, 980, 982, 983, 985, 989, 992, 993, 994, 1001, 1005, 1009, 1016, 1018, 1026, 1028, 1029, 1031, 1040, and 1041. An even greater number had higher on-target activity and a higher specificity ratio, specifically, engineered CasX proteins 977, 978, 980, 982, 983, 985, 989, 992, 993, 994, 996, 999, 1000, 1001, 1005, 1006, 1009, 1014, 1016, 1018, 1026, 1028, 1029, 1031, 1040, and 1041. Such engineered CasX proteins are therefore interpreted to be highly active and highly specific.

Taken together, the results described herein demonstrate that mutations to CasX 515 can be introduced into the sequence that result in engineered CasX with improved gene editing activity and/or specificity.

What is claimed is:

1. A system comprising an engineered CasX protein and a guide nucleic acid (gRNA), wherein the gRNA has a scaffold comprising a sequence selected from the group consisting of SEQ ID NOS: 465-466, or a sequence having at least about 90% sequence identity thereto, and a targeting sequence complementary to a proprotein convertase subtilisin/kexin Type 9 (PCSK9) gene target nucleic acid sequence.

2. The system of claim 1, wherein the gRNA comprises the sequence of SEQ ID NO: 466.

3. The system of claim 1, wherein the PCSK9 gene encodes a protein having the wild-type sequence of SEQ ID NO: 543.

4. The system of claim 1, wherein the PCSK9 gene comprises one or more mutations.

5. The system of claim 1, wherein the targeting sequence of the gRNA is complementary to a sequence of the PCSK9 gene selected from:
a. an exon;
b. a splice-acceptor site of an exon;
c. a splice-donor site;
d. intron-exon junction;
e. regulatory element
f. a 3′ untranslated region; and
g. a 5′ untranslated region.

6. The system of any one of claim 1, wherein the targeting sequence of the gRNA comprises a sequence selected from the group consisting of the sequences of SEQ ID NOS: 544-665 and 2016, or a sequence having at least about 90% sequence identity thereto.

7. (canceled)

8. The system of claim 1, wherein the gRNA is chemically modified.

9. (canceled)

10. The system of claim 8, wherein the chemically modified gRNA comprises a sequence of SEQ ID NO: 13759 or 13769, or a sequence having at least about 90% sequence identity thereto, wherein the non-targeting 20 nucleotides at the 3′ end are replaced with a targeting sequence complementary to the PCSK9 target nucleic acid to be modified.

11-30. (canceled)

31. The system of any one of claim 1, wherein the targeting sequence of the gRNA has 1, 2, 3, 4, or 5 nucleotides removed from the 3′ end of the targeting sequence.

32-67. (canceled)

68. A nucleic acid comprising a sequence that encodes the gRNA of claim 1.

69-76. (canceled)

77. A lipid nanoparticle (LNP) comprising the gRNA of claim 1.

78. A lipid nanoparticle comprising the nucleic acid of claim 68.

79-81. (canceled)

82. A vector comprising the nucleic acid of claim 68.

83-84. (canceled)

85. The vector of claim 82, wherein the vector is selected from the group consisting of a retroviral vector, a lentiviral vector, an adenoviral vector, an adeno-associated viral (AAV) vector, a herpes simplex virus (HSV) vector, a plasmid, a minicircle, a nanoplasmid, a DNA vector, and an RNA vector.

86-89. (canceled)

90. A pharmaceutical composition comprising
a. the system of claim 1
and one or more pharmaceutically suitable excipients.

91-95. (canceled)

96. A method of modifying a PCSK9 gene in a population of cells in vitro or ex vivo, the method comprising introducing into cells of the population a system comprising an engineered CasX protein and a gRNA, wherein the gRNA has a scaffold comprising a sequence selected from the group consisting of SEQ ID NOS: 465-466, or a sequence having at least about 90% sequence identity thereto, and a targeting sequence complementary to a proprotein convertase subtilisin/kexin Type 9 (PCSK9) gene target nucleic acid sequence;
wherein the PCSK9 gene of the cells targeted by the gRNA is modified.

97-109. (canceled)

110. A method of treating a PCSK9-related disease in a subject in need thereof, comprising modifying a PCSK9 gene in cells of the subject, the modifying comprising contacting said cells with a therapeutically effective dose of the system of claim 1 wherein the PCSK9 gene of the cells of the subject targeted by the gRNA are modified by the engineered CasX protein.

111-127. (canceled)

128. A system comprising an engineered CasX protein and a gRNA, wherein the engineered CasX protein comprises a target strand loading (TSL) domain comprising the sequence of amino acids 811 to 920 of SEQ ID NO: 2 or a sequence with at least 95% sequence identity thereto and comprising an amino acid substitution of V826M relative to SEQ ID NO: 2.

129. The system of claim 128, wherein the engineered CasX protein comprises an oligonucleotide binding domain 1 (OBD-1) comprising the sequence of amino acids 2 to 57 of SEQ ID NO: 2 or a sequence with at least 95% sequence identity thereto and comprising an amino acid substitution of 14G relative to SEQ ID NO: 2.

130. The system of claim 128, wherein the engineered CasX protein comprises a sequence selected from the group consisting of SEQ ID NOS: 5-7, 72, 293, 14135, 14225, 14231, 14237, 14243, and 14256, or a sequence having at least about 90% sequence identity thereto.

131. The system of claim 128, comprising one or more nuclear localization signals (NLS) linked at or near the N-terminus of the engineered CasX protein, at or near the C-terminus of the engineered CasX protein, or both, wherein the NLS comprises the sequence of a simian virus 40 (SV40) NLS of SEQ ID NO: 361 or a c-MYC NLS of SEQ ID NO: 363.

132. A nucleic acid comprising a sequence that encodes the engineered CasX protein of claim 128.

133. The nucleic acid of claim 132, wherein the nucleic acid is an mRNA sequence selected from the group consisting of SEQ ID NOS: 13740-13743 and SEQ ID NOS: 13744-13747.

134. A lipid nanoparticle comprising the nucleic acid of claim 132.

135. A vector comprising the nucleic acid of claim 132.

136. A host cell comprising the vector of claim 135.
